(19) United States
(12) Patent Application Publication
(10) Pub. No.: US 2026/0152767 A1
(43) Pub. Date: Jun. 4, 2026

(54) COMPOSITIONS, METHODS, AND SYSTEMS FOR DNA MODIFICATION

(71) Applicant: The Trustees of Columbia University in the City of New York, New York, NY (US)

(72) Inventors: Samuel Henry Sternberg, New York, NY (US); Tanner Wiegand, New York, NY (US); Florian T. Hoffmann, New York, NY (US); Matthew Walker, New York, NY (US)

(21) Appl. No.: 19/462,506

(22) Filed: Jan. 28, 2026

Related U.S. Application Data
(63) Continuation of application No. PCT/US2024/040027, filed on Jul. 29, 2024.
(60) Provisional application No. 63/516,382, filed on Jul. 28, 2023.
(60) Provisional application No. 63/604,616, filed on Nov. 30, 2023.

(51) Int. Cl. C12N 15/90; C12N 9/22; C12N 15/11

(52) U.S. Cl. CPC C12N 15/907; C12N 9/224; C12N 15/11; C12N 2310/20; C12N 2800/90

(57) ABSTRACT

Provided herein are compositions, methods, and systems for DNA modification. In particular, provided herein are compositions, and systems comprising TnpB-like nuclease-dead repressors (dTnpB/TldRs), dCas12f or dCas12f-like proteins, and/or a TnpB-transposase fusion proteins and methods using thereof.

### CROSS-REFERENCE TO RELATED APPLICATIONS

This application is a continuation of PCT International Application No. PCT/US2024/040027, filed Jul. 29, 2024, which claims the benefit of U.S. Provisional Application Nos. 63/516,382, filed Jul. 28, 2023, and 63/604,616, filed Nov. 30, 2023, the contents of which are herein incorporated by reference in their entirety.

### STATEMENT REGARDING FEDERALLY SPONSORED RESEARCH OR DEVELOPMENT

This invention was made with government support under 2239685 awarded by the National Science Foundation. The government has certain rights in the invention.

### FIELD

The present disclosure relates to compositions, methods, and systems for DNA modification. In particular the present disclosure provides compositions, and systems comprising TnpB-like nuclease-dead repressors (dTnpB/TldRs), dCas12f or dCas12f-like proteins, and/or TnpB-transposase fusion proteins and methods using thereof.

### SEQUENCE LISTING STATEMENT

The content of the electronic sequence listing titled COLUM_42528_601_SequenceListing.xml (Size: 8,375,143 bytes; and Date of Creation: Jul. 29, 2024) is herein incorporated by reference in its entirety.

### BACKGROUND

DNA transposition is a ubiquitous phenomenon occurring in all kingdoms of life during which discrete segments of DNA called transposons move from one genomic location to another. Insertion sequences (IS) are the simplest autonomous transposable elements. While they tend to be short (<2.5 kb) and carry only those genes needed for transposition, if placed flanking a DNA segment, many are able to mobilize the intervening genes. ISs can be classified into groups or families based on the general features of their DNA sequences and associated transposases. Insertion sequences of IS200/IS605 family contain the genes for their transposition and its regulation: a TnpA transposase, which is essential for mobilization, and an accessory gene, e.g., TnpB or IscB, which are evolutionary ancestors to CRISPR-Cas9 and Cas12 enzymes. These transposon components offer an expansion on genome editing options.

### SUMMARY

Disclosed herein are engineered systems comprising a TldR protein, or one or more nucleic acids encoding thereof; and at least one guide RNA, or one or more nucleic acids encoding thereof, wherein the at least one guide RNA is complementary to at least a portion of a target nucleic acid. In some embodiments, the system is a cell-free system.

In some embodiments, the TldR protein comprises an amino acid sequence having at least 70% identity to any of SEQ ID NOs: 1-508 and 1768-5926. In some embodiments, the TldR protein comprises an amino acid sequence as shown in the Table below or Table 5. In some embodiments, the TldR protein comprises an amino acid sequence of SEQ ID NOs: 1-508 and 1768-5926. In some embodiments, the TldR protein is linked or fused to one or more effector polypeptides.

In some embodiments, the at least one guide RNA is provided on an omega RNA.

Also disclosed herein are engineered systems comprising a dCas12f or dCas12f-like protein, or one or more nucleic acids encoding thereof; and at least one guide RNA, or one or more nucleic acids encoding thereof, wherein the at least one guide RNA is complementary to at least a portion of a target nucleic acid. In some embodiments, the system is a cell-free system.

In some embodiments, the dCas12f or dCas12f-like protein comprises an amino acid sequence having at least 70% identity to any sequence in Table 7. In some embodiments, the dCas12f or dCas12f-like protein comprises an amino acid sequence having at least 70% identity to any of SEQ ID NOs: 6026-6042. In some embodiments, the dCas12f or dCas12f-like protein comprises an amino acid sequence of SEQ ID NOs: 6026-6042. In some embodiments, the dCas12f or dCas12f-like protein is linked or fused to one or more effector polypeptides.

In some embodiments, the engineered system further comprises an RpoE protein. In some embodiments, the RpoE protein comprises an amino acid sequence having at least 70% identity to any of SEQ ID NOs: 6043-6059. In some embodiments, the RpoE protein comprises an amino acid sequence of SEQ ID NOs: 6043-6059. In some embodiments, the RpoE protein is linked or fused to one or more effector polypeptides.

Also disclosed herein are engineered systems comprising a TnpB-transposase fusion protein, or one or more nucleic acids encoding thereof; and at least one guide RNA, or one or more nucleic acids encoding thereof, wherein the at least one guide RNA is complementary to at least a portion of a target nucleic acid. In some embodiments, the system is a cell-free system.

In some embodiments, the TnpB-transposase fusion protein comprises an amino acid sequence having at least 70% identity to any of SEQ ID NOs: 1453-1539. In some embodiments, the TnpB-transposase fusion protein comprises an amino acid sequence of SEQ ID NOs: 1453-1539. In some embodiments, the dCas12f or dCas12f-like protein is linked or fused to one or more effector polypeptides.

In some embodiments, the system further comprises a donor nucleic acid, wherein the donor nucleic acid comprises a cargo nucleic acid sequence flanked by at least one transposon end sequence. In some embodiments, the system further comprises a target nucleic acid.

In some embodiments, the systems further comprise a target nucleic acid.

Also disclosed herein are protein conjugates comprising a TldR protein and one or more effector polypeptides. In some embodiments, the TldR protein comprises an amino acid sequence having at least 70% identity to any of SEQ ID NOs: 1-508 and 1768-5926. In some embodiments, the TldR protein comprises an amino acid sequence of SEQ ID NOs: 1-508 and 1768-5926. In some embodiments, the TldR protein is linked or fused to one or more effector polypeptides. In some embodiments, the TldR protein is separated from the one or more effector polypeptides by a linker.

Also disclosed herein are protein conjugates comprising a dCas12f or dCas12f-like protein and one or more effector polypeptides. In some embodiments, the dCas12f or dCas12f-like protein comprises an amino acid sequence having at least 70% identity to any sequence in Table 7. In some embodiments, the dCas12f or dCas12f-like protein comprises an amino acid sequence having at least 70% identity to any of SEQ ID NOs: 6026-6042. In some embodiments, the dCas12f or dCas12f-like protein comprises an amino acid sequence of SEQ ID NOs: 6026-6042. In some embodiments, the dCas12f or dCas12f-like protein is linked or fused to one or more effector polypeptides. In some embodiments, the dCas12f or dCas12f-like protein is separated from the one or more effector polypeptides by a linker.

Further disclosed are compositions and cells comprising an engineered system or protein conjugate as described herein. In some embodiments, the cell is a prokaryotic cell. In some embodiments, the cell is a eukaryotic cell. In some embodiments, the cell is a mammalian cell. In some embodiments, the cell is a human cell.

Additionally disclosed are methods for DNA modification comprising contacting a target nucleic acid sequence with a system or protein conjugate as described herein. In some embodiments, the target nucleic acid sequence is flanked on the 5′ end by a transposon-adjacent motif (TAM) sequence.

Additionally disclosed are methods for nucleic acid modification and integration. In some embodiments, the methods comprise contacting a target nucleic acid with a system, or composition thereof, as disclosed herein.

In some embodiments, the target nucleic acid sequence is in a cell. In some embodiments, contacting a target nucleic acid sequence comprises introducing the system into the cell. In some embodiments, the cell is a prokaryotic cell. In some embodiments, the cell is a eukaryotic cell (e.g., a mammalian cell, a human cell).

In some embodiments, introducing the system into the cell comprises administering the system to a subject. In some embodiments, administering comprises in vivo administration. In some embodiments, the administering comprises transplantation of ex vivo treated cells comprising the system.

Also provided are methods for treating a disease or disorder in a subject comprising administering to the subject in need thereof a system, or composition thereof, as described herein. In some embodiments, the subject is human. In some embodiments, the system or composition comprises a donor nucleic acid encoding a therapeutic gene product or a wild-type or corrected version of a disease-associated gene.

Further provided are methods for inactivating a microbial gene, the method comprising introducing into one or more cells a system, or a composition thereof, as described herein. In some embodiments, the gRNA is specific for a target site that is proximal to the microbial gene and the system or composition modifies the microbial gene. In some embodiments, the system or composition inserts a donor nucleic acid within the microbial gene. In some embodiments, the microbial gene is a bacterial antibiotic resistance gene, a virulence gene, or a metabolic gene. In some embodiments, the one or more cells are bacterial cells.

Additionally provided are methods for modifying a target nucleic acid in a plant cell comprising providing to the plant, or a plant cell, seed, fruit, plant part, or propagation material of the plant a system, or a composition thereof, as described herein. In some embodiments, the system or composition inserts a donor nucleic acid within the target nucleic acid. In some embodiments, the donor nucleic acid comprises a gene product.

Other aspects and embodiments of the disclosure will be apparent in light of the following detailed description and accompanying figures.

### BRIEF DESCRIPTION OF THE DRAWINGS

FIGS. 1A-1D show bioinformatic identification of naturally occurring, nuclease-deficient TnpB homologs. FIG. 1A, Canonical TnpB proteins are encoded by bacterial transposons known as IS elements, and exhibit RNA-guided nuclease activity that maintains transposons at sites of excision during transposition (left). Domestication of mpB genes led to the evolution of diverse CRISPR-associated cas12 derivatives, with diverse functions and mechanisms (right). LE, transposon left end; RE, right end; ωRNA (SEQ ID NO: 1540), transposon-encoded guide RNA; crRNA, CRISPR RNA. FIG. 1B, Phylogenetic tree of TnpB proteins, with previously studied homologs and newly identified TnpB-like nuclease-dead repressor (TldR) proteins highlighted. The rings indicate RuvC DED active site intactness (inner), TnpA transposase association (middle) and protein size (outer). FIG. 1C, Multiple sequence alignment of representative TnpB and TldR sequences (SEQ ID NOs: 1541-1562), highlighting deterioration of RuvC active site motifs and loss of the C-terminal Zinc-finger (ZnF)/RuvC domain. FIG. 1D, Empirical (DraTnpB) and predicted AlphaFold structures of TnpB and TldR homologs marked with an asterisk in FIG. 1C, showing progressive loss of the active site catalytic triad.

FIGS. 2A-2C show tldR genes are strongly associated with diverse non-transposon genes and encoded in prophages. FIG. 2A, Genomic architecture of well-studied transposons that encode TnpB (top), and of novel regions that encode TldR proteins (bottom) in association with prophage-encoded fliC_{P} (left), oppF and ABC transporter operons (middle), and a transcriptional regulator (csrA) of an accompanying fliC (right). FIG. 2B, Comparison of a representative fliC_{P}-tldR locus with a closely related Enterobacter kobei strain reveals that the entire locus is encoded within the boundaries of the prophage element, with identifiable recombination sequences (attL/attR/attB). FIG. 2C, Phylogenetic tree of fliC_{P}-associated TldR proteins from FIG. 2A, together with closely related TnpB proteins that contain intact RuvC active sites. The rings indicate RuvC DED active site intactness (inner), prophage association (middle), fliC_{P} association (middle), and TldR/TnpB domain composition (outer). Prophage association was defined as true if the homolog was encoded within 20 kbp of five or more genes with a phage annotation; fliC_{P} association was defined as true if the homolog was encoded within three ORFs of a fliC homolog. Homologs marked with a blue square (TnpB) or green circle (TldR) were tested in heterologous experiments.

FIGS. 3A-3G show TldR proteins are encoded next to gRNAs that target conserved genomic sites. FIG. 3A, Bioinformatic strategies to investigate tldR/tnpB loci, including comparative genomics, searching within the ISfinder database, gRNA prediction using covariance models, and target prediction using BLAST. FIG. 3B, Representative tnpB locus and an isogenic locus above that lacks the IS element. Comparison of both sequences reveals the putative TAM recognized by TnpB, which flanks the transposon LE, and the guide portion of the ωRNA, which flanks the transposon RE. Isogenic sequence, SEQ ID NO: 1563; tnpB locus SEQ ID NOs: 1564 and 1565. FIG. 3C, Schematic of a representative fliC_{P}-tldR locus from Enterobacter cloacae (top), and bioinformatics approach to predict the gRNA sequence using both CM search and comparison to related tnpB loci (SEQ ID NOs: 1566-1570). This analysis identified the putative scaffold and guide portions of TldR- and TnpB-associated gRNAs (bottom). FIG. 3D, Analysis of the guide sequence (SEQ ID NO: 1571) from the EclTldR-associated gRNA in FIG. 3C revealed a putative genomic target near the predicted promoter of a distinct (host) copy of fliC located ˜1 Mbp away (middle). The magnified schematic at the bottom shows the predicted TAM and gRNA-target DNA base-pairing interactions relative to the fliC start codon (SEQ ID NO: 1572 and 1573). FIG. 3E, Annotated −10 and −35 promoter elements upstream of fliC recognized by FliA/σ²⁸ in E. coli K12; SEQ ID NO: 1574 (top), and WebLogos of predicted guides and genomic targets associated with diverse fliC_{P}-associated TldRs from FIG. 2C (bottom). FIGS. 3F-3G, Published RNA-seq data for Enterobacter cloacae (FIG. 3F) and Enterococcus faecalis (FIG. 3G) reveal evidence of native tldR and gRNA expression for fliC_{P}- and oppF-associated TldRs, respectively. The predicted gRNAs from CM analyses are indicated; unique genome-mapping reads are shown as overlays of three replicates.

FIGS. 4A-4H show TldRs are RNA-guided DNA-binding proteins capable of programmable transcriptional repression. FIG. 4A, RNA immunoprecipitation sequencing (RIP-seq) data from a fliC_{P}-associated TldR homolog from Enterobacter hormaechei (EhoTldR) reveals the boundaries of a mature gRNA containing a 16-nt guide sequence. Reads were mapped to the TldR-gRNA expression plasmid (SEQ ID NOs: 1575 (left) and 1576 (right)); an input control is shown. FIG. 4B, Schematic of chromatin immunoprecipitation DNA sequencing (ChIP-seq) approach to investigate RNA-guided DNA binding for TldR candidates (top), and representative ChIP-seq data for four homologs revealing strong enrichment at the expected genomic target site and a prominent off-target (bottom). FIG. 4C, Magnified view of ChIP-seq peaks at the labeled off-target site in FIG. 4B, which corresponds to a TAM and partially matching target sequence at the promoter of E. coli K12 fliC (SEQ ID NOs: 1577 and 1578). FIG. 4D, Analysis of conserved motifs bound by the indicated TldR homolog using MEME ChIP, which reveals specificity for the TAM and a ˜6-nt seed sequence (SEQ ID NO: 1579 shown below). The number of peaks and percentage of total called peaks contributing to each motif is indicated; low occupancy positions were manually trimmed from motif 5′ ends. FIG. 4E, Schematic of E. coli-based plasmid interference assay using pEffector and pTarget (left), and bar graph plotting surviving colony-forming units (CFU) for the indicated conditions and proteins (right). TnpB nucleases cause robust cell death, whereas TldR homologs have no effect on cell viability, indicating a lack of DNA cleavage activity. EV, empty vector; M, TnpB mutant; NT, non-targeting guide; T, targeting guide. Bars indicate mean+s.d. (n=3). FIG. 4F, Alternative models of TldR-mediated transcriptional repression by blocking either transcription initiation or elongation by RNAP (blue). FIG. 4G, Schematic of RFP repression assay in which gRNAs were designed to target either the top or bottom strand of a promoter driving rfp expression (left), and bar graph plotting normalized RFP fluorescence for the indicated conditions. EV, empty vector; NT, non-targeting guide; Top/Btm, gRNA targeting the top or bottom strand. Bars indicate mean±s.d. (n=3). FIG. 4H, Experiments and data shown as in FIG. 4G, but with guides targeting the top/bottom strand within the 5′ UTR, downstream of the promoter. Results with nuclease-dead dCas12 and dCas9 are shown for comparison. Bars indicate mean±s.d. (n=3 for TldR; n=6 for dCas12/dCas9).

FIGS. 5A-5K show flagellin-associated TldRs repress host flagellin gene expression in native clinical Enterobacter strains. FIG. 5A, Schematic of the flagellar assembly spanning the inner membrane (IM), cell wall (CW), and outer membrane (OM). The flagellin (FliC), hook (FlgE), stator-interacting (FliL), and flagellar cap (FliD) proteins are indicated. FliC filaments typically comprise several thousand subunits, are 5-20 μm in length, and are known receptors of flagellotropic phages. FIG. 5B, Surface representation of E. coli FliC (PDB: 7SN4) colored by domains, showing both a single monomer and filament cross section (left). Surface representations of ColabFold-predicted prophage FliC_{P} (middle) and host FliC (right) structures from Enterobacter cloacae, colored with AL2CO conservation scores calculated from the multiple sequence alignment (MSA) shown in FIG. 5C. FIG. 5C, MSA of TldR-associated FliC_{P} and TldR-targeted FliC proteins, showing the strongly conserved DO-1 domains and hypervariable D2-3 domains. FIG. 5D, Schematic of Enterobacter strains selected for RNA-seq analysis (top), and expression data plotted as transcripts per million (TPM) for fliC_{P} (when present) and host fliC and flil). The presence/absence of fliC_{P}-tldR loci is indicated below the graph. Bars indicate mean±s.d. (n=3). FIG. 5E, Schematic of Enterobacter cloacae mutants generated by recombineering (left), and RT-qPCR analysis of host fliC expression levels normalized to the WT strain with cmR marker. Any deletion of tldR or substitution with a non-targeting (NT) gRNA leads to fliC de-repression. Bars indicate mean±s.d. (n=3). FIG. 5F, RNA-seq coverage at the host fliC locus for the indicated strains in e, showing de-depression with the NT-gRNA. FIG. 5G, Volcano plot showing differential gene expression analysis for the WT and NT-gRNA strains in FIG. 5F. Genes with a log 2 (fold change) ≥1 and an adjusted p-value <0.05 are highlighted in red. FIG. 5H, Magnified view of data in FIG. 5F, showing the TAM/target overlap with predicted FliA/σ²⁸ promoter elements inferred from E. coli K12 data. FIG. 5I, Predicted AlphaFold structure of TldR bound to target DNA (left) compared to experimental structure of RNAP (grey) and FliA/σ²⁸ (green) bound to promoter DNA (right). FIG. 5J, Comparison of promoter motifs for host fliC and prophage fliC_{P} alongside the FliA/σ²⁸ motif from Tomtom analysis. This analysis suggests that fliC_{P} is expressed similarly as fliC, while harboring conserved mutations (red) in the TAM and seed sequence that preclude self-targeting by its associated TldR. FIG. 5K, Model for the role of TldR in RNA-guided repression of host fliC upon temperate phage infection, leading to the selective expression and generation of phage-encoded flagellin (FliC_{P}) filaments.

FIGS. 6A-6C show phylogeny and RuvC nuclease domain analysis of oppf-associated TldRs. FIG. 6A, Phylogenetic tree of oppF-associated TldR proteins from FIG. 2A, together with closely related TnpB proteins that contain intact RuvC active sites. The rings indicate RuvC DED active site intactness (inner) and TldR/TnpB domain composition (outer). Homologs marked with an orange square (TnpB) or purple circle (TldR) were tested in heterologous experiments. FIG. 6B, Multiple sequence alignment of representative TnpB and TldR sequences from FIG. 6A, highlighting deterioration of RuvC active site motifs and loss of the C-terminal Zinc-finger (ZnF)/RuvC domain. SEQ ID NO: 1580-1607. FIG. 6C, Empirical (DraTnpB) and predicted AlphaFold structures of TnpB and TldR homologs marked with an asterisk in FIG. 6B, showing progressive loss of the active site catalytic triad.

FIGS. 7A-7C show diverse prophages encode fliC_{P}-associated tldR genes. FIG. 7A, Genomic architecture of representative prophage elements whose boundaries could be identified by comparing to closely related isogenic strains. In each example, the prophage-containing strain is shown above the prophage-less strain, with species/strain names and NCBI genomic accession IDs indicated. Sequences flanking the left (5′) and right (3′) ends are highlighted in purple and yellow, respectively, together with their percentage sequence identifies calculated using BLASTn. FIG. 7B, Alignment of distinct prophage elements, constructed using Mauve. Empty boxes represent open reading frames, and windows show sequence conservation for regions compared between prophage genomes with lines. Putative gene functions are shown below sequence conservation windows for the fliC_{P}-tldR-encoding prophage from Enterobacter AR_163 (bottom). FIG. 7C, DNA sequence identities between the prophages in FIG. 7A, calculated with BLASTn. Identities were calculated as total matching nucleotides across the two genomes being compared, divided by the length of the query prophage genome.

FIGS. 8A-8C show RIP-seq reveals that some oppF-associated TldR proteins use short, 9-11-nt guides. FIG. 8A, RNA immunoprecipitation sequencing (RIP-seq) data for an oppF-associated TldR homolog from Enterococcus faecalis (Efa1TldR) reveals the boundaries of a mature gRNA containing a 9-nt guide sequence. Reads were mapped to the TldR-gRNA expression plasmid (SEQ ID NOs: 1608 (left) and 1609 (right)); an input control is shown. FIG. 8B, Published RNA-seq data for Enterococcus faecalis V583 reveals similar gRNA boundaries, including an ˜11-nt guide. SEQ ID NOs: 1610 (left) and 1611 (right). FIG. 8C, RIP-seq data as in FIG. 8A for a second biological replicate of FfaITldR, further corroborating the observed ˜9-11-nt guide length. SEQ ID NOs: 1612 (left) and 1613 (right).

FIGS. 9A-9E show oppF-associated TldRs target conserved genomic sequences that overlap with promoter elements driving oppA expression. FIG. 9A, Schematic of original (left) and new (right) search strategy to identify putative targets of gRNAs used by oppF-associated TldRs. Key insights resulted from the use of TAM and a shorter, 9-nt guide. FIG. 9B, Analysis of the guide sequence from the Efa1TldR-associated gRNA in FIG. 8 revealed a putative genomic target near the predicted promoter of oppA encoded within the same ABC transporter operon immediately adjacent to the tldR gene. The magnified schematics at the bottom show the predicted TAM and gRNA-target DNA base-pairing interactions for two representatives (Efa1TldR and EceTldR), in which the gRNAs target opposite strands. Promoter elements predicted with BPROM are shown as brown squares. SEQ ID NOs: 1614-1619, top to bottom in schemes. FIG. 9C, WebLogos of predicted guides and genomic targets associated with diverse oppF-associated TldRs highlighted in FIG. 18A. FIG. 9D, Schematic of the oppF-tldR genomic locus (left) alongside the predicted function of OppA as a solute binding protein that facilitates transport of polypeptide substrates from the periplasm to the cytoplasm, in complex with the remainder of the ABC transporter apparatus. CM, cell membrane. FIG. 9E, Published RNA-seq data for Enterococcus faecium AUS0004 (Michaux, C. et al. Front Cell Infect Microbiol 10, 600325 (2020)), highlighting the oppA transcription start site (TSS). The predicted gRNA guide sequence (grey; SEQ ID NO: 5927) is shown beneath the putative TAM (yellow) and target (purple) sequences (in SEQ ID NO: 1620), with guide-target complementarity represented by grey circles.

FIG. 10 shows oppF-associated TldR homologs may target additional sites across the genome. Schematic of Enterococcus cecorum genome and inset showing the oppf-tldR locus (top), with additional putative targets of the gRNA, other than the oppA promoter, numbered and highlighted in yellow along the genomic coordinate. A magnified view for each numbered target is shown below, with TAMs in yellow, prospective targets in purple, and TldR gRNA guide sequences in grey. Grey circles (right) represent positions of expected guide-target complementarity. SEQ ID NOs: 1621-1634, top to bottom.

FIGS. 11A-11B show that genome-wide binding data from ChIP-seq experiments suggests a high mismatch tolerance for some TldR homologs. FIG. 11A, Genome-wide ChIP-seq profiles for the indicated fliC_{P}-associated TldR homologs, normalized to the highest peak within each dataset. The magnified insets at the bottom show the off-target sequences (grey; SEQ ID NOs: 1635 and 1637) compared to the intended (engineered; SEQ ID NOs: 1636 and 1638) on-target sequence (purple), with TAMs in yellow. Off-target #3 has no clear TAM-flanked off-target sequence but is intriguingly located at a tRNA locus, and binding was observed for diverse fliC_{P}- and oppF-associated TldRs that recognized distinct TAMs. The phylogenetic tree at right indicates the relatedness of the tested and labeled homologs. FIG. 11B, Results for the indicated oppf-associated TldR homologs, shown as in FIG. 11A. Off-target sequences (grey; SEQ ID NOs: 1639, 1641, and 1643) and intended (engineered; SEQ ID NOs: 1640, 1642, and 1644)

FIGS. 12A-12D show plasmid interference assays confirming that TldR homologs lack detectable nuclease activity. FIG. 12A, Schematic of E. coli-based plasmid interference assay using pEffector and pTarget. FIG. 12B, Representative dilution spot assays for GstTnpB3 and synthetically inactivated RuvC mutant (D196A), showing the entire plate (left) and the magnified area of plating. Transformants were serially diluted, plated on selective media, and cultured at 37° C. for 16 h. Colony visibility was enhanced by inverted the colors and increasing contrast/brightness. FIG. 12C, Dilution spot assays for the indicated fliC-associated TldR homologs and closely related TnpB homologs. Non-targeting (NT) gRNA controls are shown at the bottom, and the phylogenetic tree indicates the relatedness of the tested proteins. FIG. 12D, Results for the indicated oppF-associated TldR and TnpB homologs, shown as in FIG. 12C.

FIGS. 13A-13B show RFP repression assays reveal variable abilities of TldR homologs to block transcription elongation. FIG. 13A, Schematic of RFP repression assay adapted from FIG. 4G (left), in which gRNAs were designed to target either the top or bottom strand within the 5′ UTR of RFP, downstream of the promoter. The phylogenetic trees (right) indicate the relatedness of the tested and labeled homologs. FIG. 13B, Bar graph plotting normalized RFP fluorescence for the indicated conditions and TldR homologs. EV, empty vector; NT, non-targeting guide. Bars indicate mean±s.d. (n=3).

FIGS. 14A-14C show Enterobacter RNA-seq data confirming the native expression of gRNAs from fliC_{P}-tldR loci. FIG. 14A, RNA-seq read coverage from three Enterobacter strains that natively encode fliC_{P}-tldR loci, revealing clear peaks associated with mature gRNAs containing ˜95-97-nt scaffolds (SEQ ID NOs: 1645-1647 shown top, left to right) and 16-nt guides (SEQ ID NO: 1648-1650 shown bottom, left to right). Data from three biological replicates are overlaid. FIG. 14B, Predicted secondary structure and sequence (SEQ ID NO: 1651) of the gRNA associated with EhoTldR. FIG. 14C, Multiple sequence alignment of the DNA encoding gRNA scaffold sequences for representative fliC_{P}-associated TldRs, with conserved positions colored in darker blue (SEQ ID NOs: 1652-1658).

FIGS. 15A-15E show Enterobacter RNA-seq data confirming the overlap between TldR-gRNA binding sites and host fliC promoters. FIG. 15A, RNA-seq read coverage in the host fliC promoter/5′-UTR region for four Enterobacter strains, with labeled TAM and target sequences highlighted upstream of the TSS. Strain AR136 (top left) does not encode a fliC_{P}-tldR locus; note the distinct expression levels, measured via relative counts per million (CPM). FIG. 15B, Alignment of host fliC promoter regions for the strains shown in FIG. 15A compared to E. coli K12, with percent sequence identities indicated on the right. Reported FliA/σ²⁸ promoter elements from E. coli K12 are shown below the alignment. SEQ ID NOs: 1660-1664, grey sequence as SEQ ID NO: 1659. FIG. 15C, RNA-seq read coverage in the prophage-encoded fliC_{P} promoter/5′-UTR region for two representative Enterobacter strains, confirming the predicted TSS. SEQ ID NO: 1665. FIG. 15D, Schematic of multiple sequence alignment (MSA) of the promoter region driving fliC_{P} gene expression, across six verified prophages described in FIG. 7. FIG. 15E, Magnified MSA for the indicated region in FIG. 15D, highlighting the region that was queried for MEME motif detection. SEQ ID NOs: 1666-1671.

FIGS. 16A-16B show fliC_{P}-tldR loci are encoded within prophages and phage genomes. FIG. 16A, Genetic architecture of a 40 kbp window of bacterial genomes that encode fliC_{P}-tldR loci (center). fliC_{P} and tldR genes are colored in light blue and green, respectively, and genes with Eggnog annotations containing the word “phage” or “viridae” are colored in orange; all other annotated genes are shown in grey. Each locus is annotated with NCBI accession IDs and genomic coordinates; “_rc” indicates that annotations for the reverse complement sequence are shown. FIG. 16B, Two metagenome-assembled phage genomes encode fliC_{P}-tldR loci. NCBI accessions are shown on the left.

FIG. 17 shows TldR-associated gRNA sequences identified using covariance models (SEQ ID NOs: 1672-1694). Phylogenetic tree of fliC- and oppF-associated TldR homologs alongside related TnpB proteins (top), and scaffold/guide junctions for putative TldR-associated gRNAs identified using covariance models (bottom). Matches to the covariance model are shaded, and protein accession IDs are shown at the right.

FIGS. 18A-18C show RIP-seq data for additional oppF-associated TldR proteins revealing variable gRNA substrates. FIG. 18A, RNA immunoprecipitation sequencing (RIP-seq) data for oppF-associated TldR homologs from Enterococcus cecorum (EceTldR) and Enterococcus casseliflavus (EcaTldR) indicates variable length guide sequences. Reads were mapped to each respective expression plasmid. SEQ ID NOs: 1695-1698. FIG. 18B, RIP-seq data for EmuTldR and Ffa2TldR, shown as in FIG. 18A. FIG. 18C, RIP-seq data for EsaTldR, shown as in a. Enrichment for the gRNA region was not observed, relative to the input control.

FIG. 19 shows pairwise identity matrices for representative TldR proteins and related TnpB homologs. Pairwise sequence identities at the amino acid level were calculated for each of the representative TldRs and TnpBs highlighted in FIG. 6A, for fliC_{P}-associated (top) and oppF-associated (bottom) clades.

FIGS. 20A-20F show genome-wide binding data from ChIP-seq experiments for additional TldR homologs. FIG. 20A, Genome-wide ChIP-seq profiles for the indicated fliC_{P}-associated TldR homologs, normalized to the highest peak within each dataset except for the input control (top). The magnified inset at the left shows enrichment at the genomically-integrated, gRNA-matching target site. FIG. 20B, Analysis of conserved motifs bound by the indicated TldR homolog in a using MEME ChIP, which reveals specificity for the TAM and a ˜6-nt seed sequence (SEQ ID NO: 1699). The number of peaks and percentage of total called peaks contributing to each motif is indicated; low occupancy positions were manually trimmed from motif 5′ ends. Motifs are omitted for datasets for which a high-confidence consensus could not be identified. FIG. 20C, Genome-wide ChIP-seq profiles for the indicated oppF-associated TldR homologs, shown as in FIG. 20A. FIG. 20D, Analysis of conserved motifs bound by the indicated TldR homolog in c using MEME ChIP, shown as in FIG. 20B. TAM and a seed sequence (SEQ ID NO: 1700). FIG. 20E, Genome-wide ChIP-seq profile for GstTnpB_{D196A}, shown as in FIG. 20A. FIG. 20F, Analysis of conserved motifs bound by GistTnpB_{D196A} in FIG. 20E using MEME ChIP, shown as in FIG. 20B.

FIGS. 21A-21B show comparison of TAM specificities for oppF-associated TldRs and related TnpBs, determined via ChIP-seq and comparative genomics. FIG. 21A, Phylogenetic tree showing the relatedness of labeled oppF-associated TldRs and similar TnpB homologs (left), and consensus motifs from TldR homologs using MEME ChIP, replotted from FIG. 20. TAMs and target regions are colored in yellow and purpled, respectively. FIG. 21B, Bioinformatically predicted TAMs and target sequences (SEQ ID NOs: 1701-1704) for related TnpB homologs labeled in the tree from FIG. 21A. Reference genomes used for comparative genomics analyses to predict the TAM (yellow) and target (purple) are indicated, and harbored either isogenic loci lacking the transposon IS element, or multiple copies of the same IS element.

FIG. 22 show bioinformatic identification of naturally inactive TnpB (e.g., dTnpB) protein sequences. The flow chart represents the different steps, and in some cases, software packages, that are used in order to arrive at a catalog list of nuclease-deactivated dTnpB homologs, which are prioritized for experimental testing.

FIG. 23 shows prediction and verification of dTnpB ωRNA scaffold boundaries. Analyses of RNAseq data from NCBI short read archive (SRA accessions ERR6044061, ERR6044062, ERR6044063) indicate expression of a transcript consistent with TnpB ωRNAs.

FIG. 24 shows bioinformatic identification of natural TnpB-transposase fusion proteins. Left: bioinformatic pipeline, Right (top): profile HMMs used to identify TnpB proteins, Right (bottom): transposase profile HMMs selected to filter TnpB sequences for TnpB-transposase fusion proteins.

FIG. 25 shows a phylogenetic tree of natural TnpB-transposase fusion proteins. Inner ring: taxonomy of host organism; middle ring: domain fused to TnpB/Fanzor; outer ring: relative size of fusion protein; branch tips: covariation model hits for ωRNA or left end sequences. Key shown on right.

FIG. 26 shows TnpB-transposase fusion loci with ωRNA and LE sequences identified via covariation analysis. Orange and green arrows represent open reading frames >75 amino acids (aa). Red arrows represent genes encoding TnpB-transposase fusions. Grey boxes indicate 3′ boundaries of covariation model hits for ωRNA and LE elements.

FIG. 27 shows comparison of TnpB-transposase fusion structural prediction to experimentally determined structures. Left: structure of TnpB (light indigo) from D. radiodurans (ISDra2), bound to ωRNA (salmon) and double-stranded DNA target (green and tan). Middle: clear structural homology in predicted folds of TnpB (blue) and transposase (orange) domains of a TnpB-transposase fusion protein (SCI79596.1). Right: structure of dimeric transposase (TnpA) from S. solfataricus (IS200). Protomers are shown in grey and purple.

FIG. 28 shows multiple alignment of TnpB-transposase (TnpA) fusion sequences SEQ ID NOs: 1705-1767. Top: subset of multiple sequence alignment (MSA) highlighting conservation of TnpB domain catalytic motif (DED; SEQ ID NOs: 1705-1714 (D); SEQ ID NOs: 1714-1729 (E); SEQ ID NOs: 1730-1742 (D)). Bottom: subset of MSA highlighting conservation of transposase (TnpA) domain catalytic motifs (HUH (SEQ ID NOs: 1743-1755)+Y (SEQ ID NOs: 1756-1767); U=hydrophobic residue). An exemplary TnpB-transposase fusion sequence (EEM92921.1) with conserved catalytic residues in both domains is highlighted with green arrows.

FIG. 29 shows a phylogenetic tree of csrA-associated TldR homologs and closely related TnpB proteins. TldR proteins form a monophyletic clade (green shading), suggesting that they originated from a shared ancestor. Mutations in the nuclease active site (green) that are expected to abolish DNA cleavage activity are shown in the inner ring surrounding the tree, and genetic associations with a carbon storage regulator gene (csrA; orange) and a flagellin gene (blue) are shown in the middle and outer rings, respectively. Seven candidates, which were selected to sample TldR phylogenetic diversity and cloned into expression vectors for experimental analyses, are indicated by branch symbols (red circles).

FIGS. 30A-30D show that ChIP-seq identifies putative guide sequences and target-adjacent motifs (TAMs) of csrA-associated TldRs. FIG. 30A is an example locus of a TldR protein encoded in an operon with csrA and a flagellin gene. In this locus, there are two distinct csrA genes, but many other examples encode just a single csrA gene. The gRNA region identified by RIP-seq experiments is indicated. FIG. 30B shows the genes encoding TldR proteins cloned into expression vectors with csrA, and a region comprising the putative gRNA (i.e., the 3′-end of the TldR coding sequence, plus the downstream intergenic region flanking the 3′-end of tldR). FIG. 30C shows ChIP-seq peaks from experiments with heterologous expression of OspTldR in E. coli, shown below the corresponding input tracks. Magnified insets for each of the three prominent peaks are indicated above the input track, in read. FIG. 30D shows the motif enriched in the ChIP-seq peaks shown in FIG. 30C, representing the putative TAM (yellow) and guide sequence (purple) of OspTldR. Note that the guide corresponds to the first stretch of nucleotides within the putative seed sequence.

FIGS. 31A-31C show bioinformatically identified targets of csrA-associated TldRs. FIG. 31A shows csrA-associated TldRs target a conserved, putative genomic site near the 5′-end of the coding sequence for a Flagellin gene (blue, with target site in small purple rectangle). Note that the flagellin gene may be annotated as either hag or fliC. FIG. 31B shows nucleotide-level view of putative TldR-gRNA targets for two distinct homologs on the top and bottom (Osp (SEQ ID NOs: 6114-6115) and Isp (SEQ ID NOs: 6116-6117)), showing that TAMs are consistent with ChIP-seq data in FIG. 30D. FIG. 31C is a schematic of the hypothesized role of csrA-associated TldR in the transcriptional repression of flagellin genes (Flagellin-2, bottom right)), which are distinct from the flagellin genes encoded near tldR (top left). TldR binding is expected to sterically block the progression of actively transcribing RNA polymerase (RNAP) holoenzymes, preventing expression of the flagellin-2 gene.

FIGS. 32A-32B show RIP-seq reveals csrA-associated TldR gRNA sequences. FIG. 32A shows RIP-seq coverage of reads mapping to the gRNA region of csrA-associated ldR expression vectors. Data are shown for six distinct homologs, labeled on the far right of each coverage track. The schematic at the top depicts a portion of the 3′-end tldR gene, as well as the putative scaffold region (orange) that is upstream of the putative guide sequence (purple). The corresponding regions for each individual homolog are indicated, from the expression vectors tested. FIG. 32B shows the predicted secondary structure of a representative (Fba) csrA-associated TldR gRNA (bottom; SEQ ID NOs: 6118-6119), and model for RNase III-mediated gRNA processing (top right). The region drawn in black is cleaved off by RNAse II, leading to the conspicuous drop in RIP-seq coverage observed in FIG. 32A.

FIGS. 33A-33C show csrA-associated TldRs target DNA and RNA for transcriptional and translational repression. FIG. 33A shows ChIP-Seq of csrA-associated TldR components from Osp expressed in E. coli. ChIP-Seq of 3×LAG-tagged TldR reveals active DNA targeting (row 1). A panel of mutants lacking distinct components of the system (2-7) reveals that the upstream portion of the gRNA region is required (4) but that the downstream region is dispensable for targeting (5). ChIP-Seq of 3×FLAG-tagged CsrA indicates that CsrA does not target DNA in the presence or absence of TldR (8-9) FIG. 33B shows RIP-Seq of 3×FLAG-tagged Osp CsrA in E. coli heterologously expressing the upstream region of Osp fliC. CsrA is enriched ˜30-nt upstream of the fliC start codon. FIG. 33C shows CsrA enrichment by RIP-Seq corresponds to a CsrA consensus sequence (orange) within the loop of a predicted stem-loop (mfold), which encodes a central “GGA” motif for CsrA binding (blue); SEQ ID NO: 6120.

FIGS. 34A-34E show bioinformatic analysis of rpoE-associated dCas12f systems. FIG. 34A is a phylogenetic tree of 707 unique rpol-associated dCas12f homologs and closely-related Cas12f proteins. Gene associations are marked with different colors, from inner circle to outer circle: helix-turn-helix (hth, purple); Sigma factor rpoF (orange); transposase (yellow). The association with rpok is widely conserved across the collected dCas12f homologs. The 16 red dots mark diverse dCas12f homolog systems from across the phylogenetic tree that were selected for gene synthesis, cloning, and biochemical testing in E. coli. FIG. 34B is a representative native locus of an rpof-associated dCas12f system. Typically, these systems include genes encoding RpoE (dark blue) and dCas12f (light blue) immediately adjacent to one another, with a hth gene (magenta) encoded upstream, in opposite orientation. As with canonical Cas12f proteins, the gRNA (pink box with dashed lines) is encoded downstream of the dcas12f gene. Portions of the intergenic sequence in between rpok and hth are conserved and hence named ‘conserved non-coding region’ (pale blue box with dashed lines). FIG. 34C is a structural superposition of a nuclease-active (InCas12f homolog (PDB ID 7L49, dark beige) with an AlphaFold2-predicted structure of AtadCas12f (blue) reveals that the key catalytic residues (DED) are mutated and truncated in AtadCas12f, indicating the expected inability of AtadCas12f to cleave DNA (nuclease dead Cas12f, or dCas12f). Here, the first two catalytic residues of AtadCas12f are mutated while the C-terminus containing the Zinc finger in (InCas12f (orange) is fully absent in AtadCas12f. The UnCas12f sgRNA is colored red; target DNA is colored dark grey. FIG. 34D is a multiple sequence alignment (MSA) of three nuclease-active (InCas12f homolog amino acid sequences (SEQ ID NOs: 6121-6123) and three rpoE-associated dCas12f homologs (SEQ ID NOS: 6028, 6032, and 6033, respectively), which highlights the mutated and C-terminally truncated catalytic residues of dCas12f proteins. Key residues involved in UnCas12f dimerization, PAM recognition, and Zinc Finger motif formation are highlighted. Residues are colored at a 30% sequence identity threshold. FIG. 34E is an exemplary schematic of programmable RNA-guided gene activation by an rpoE-associated dCas12f system in complex with bacterial RNA polymerase (RNAP). The −35 and −10 promoter elements are highlighted in yellow; the core RNAP subunits are shown in shades of green. Transcription start site, TSS.

FIG. 35A is native dCas12f locus maps for 16 homolog systems for ChIP/RIP-seq. FIG. 35B is a representative plasmid layout for heterologous experiments in E. coli. FIG. 35C is a schematic of ChIP-seq and RIP-seq (SEQ ID NO: 6163). FIG. 35D is ChIP-seq genome-wide peaks. FIG. 35E is ChIP-seq MEME-ChIP TAM motifs. FIG. 35F is RIP-seq coverages (plasmid mapping), left, and RIP guide identification in 3′ end of coverage, right (SEQ ID NOs: 6124-6136).

FIG. 36A is a gRNA scaffold sequence alignment (SEQ ID NOs: 6137-6147, top to bottom). FIG. 36B is a gRNA guide sequence alignment (SEQ ID NOs: 6148-6158, top to bottom). FIG. 36C is a gRNA structure of the Ata homolog (SEQ ID NO: 6159). FIG. 36D is an Ata homolog native target site (guide is SEQ ID NO: 6160 and target is SEQ ID NO: 6161). FIG. 36E is representative dCas12f locus that is close to TonB locus.

FIG. 37A is a schematic of Ata dCas12f ChIP-seq re-targeting/re-programming (top) and Ata RpoE ChIP-seq re-targeting/re-programming demonstrates targeting along dCas12f (bottom). FIG. 37B shows RNA-seq increased signal for target 4 demonstrating target gene upregulation. FIG. 37C shows re-targeting of other dCas12f homologs (FLAG-dCas12f).

FIG. 38A shows ChIP-qPCR using plasmids with deletions and FLAG-tag attached to different protein components. All experiments were performed at target site 4. Deletion of the hth gene does not affect recruitment of dCas12f to the target site. HTH-FLAG is not recruited to the target site along dCas12f indicating it does not serve as an essential component in the system. FIG. 38B shows ChIP-seq of HTH mapping to expression plasmid (SEQ ID No: 6162). HTH-FLAG binds to the conserved non-coding region, directly upstream of the hth gene suggesting an autoregulatory function rather than involvement in RNA-guided activation of transcription. FIG. 38C shows plasmid design for gene activation assays in E. coli. Several possibilities to show gene activation in E. coli using the native Ata homolog target site or targets tiled upstream of a weak promoter. Fluorescence as well as native target gene expression (susC) can be used as the readout. Native Ata RNAP encoded on additional plasmids can be added to reconstitute a native transcription system.

### DETAILED DESCRIPTION

The disclosed systems, kits, and methods provide systems and methods for nucleic acid modification. Described herein are TnpB-like nuclease-dead repressors (TldR), dCas12f or dCas12f-like proteins, and/or a TnpB-transposase fusion proteins identified using phylogenetics, structural predictions, comparative genomics, and functional assays. These proteins employ guide RNAs to specifically target and bind nucleic acid sequences and modify gene expression.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

A “peptide” or “polypeptide” is a linked sequence of two or more amino acids linked by peptide bonds. The polypeptide can be natural, synthetic, or a modification or combination of natural and synthetic. The peptide or polypeptide may be modified by the addition of sugars, lipids or other moieties not included in the amino acid chain. The terms “polypeptide,” “oligopeptide,” “protein,” and “peptide” are used interchangeably herein. The peptide may be produced by recombinant genetic technology or chemical synthesis. The peptide may be isolated and purified by any number of standard methods including, but not limited to, differential solubility (e.g., precipitation), centrifugation, chromatography (e.g., affinity, ion exchange, and size exclusion), or by any other standard techniques known in the art.

As used herein, “conjugate” refers to the linking of two or more moieties or molecules to each other by covalent or non-covalent interactions. More specifically, the terms “protein conjugate” refer to a protein that has been modified by the addition of another moiety or molecule (e.g., another peptide, protein, or polypeptide).

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence of the present disclosure after aligning the two sequences and introducing gaps, if necessary, to achieve the maximum percent identity. A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

The term “homology” and “homologous” refers to a degree of identity. There may be partial homology or complete homology. A partially homologous sequence is one that is less than 100% identical to another sequence.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. Hybridization methods involve the annealing of one nucleic acid to another, complementary nucleic acid, e.g., a nucleic acid having a complementary nucleotide sequence. The ability of two polymers of nucleic acid containing complementary sequences to find each other and “anneal” or “hybridize” through base pairing interaction is a well-recognized phenomenon. The initial observations of the “hybridization” process by Marmur and Lane, Proc. Natl. Acad. Sci. USA, 46:453 (1960) and Doty et al., Proc. Natl. Acad. Sci. USA, 46:461 (1960), have been followed by the refinement of this process into an essential tool of modern biology. For example, hybridization and washing conditions are now well known and exemplified in Sambrook et al., supra. The conditions of temperature and ionic strength determine the “stringency” of the hybridization.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization.

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure (e.g., a stem-loop structure) may also be considered a “double-stranded nucleic acid.” For example, triplex structures are considered to be “double-stranded.” In some embodiments, any base-paired nucleic acid is a “double-stranded nucleic acid.”

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism. For the purpose of this disclosure, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the systems of the disclosure into a cell, organism, or subject by a method or route which results in at least partial localization of the system to a desired site. The systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Polypeptides and Compositions

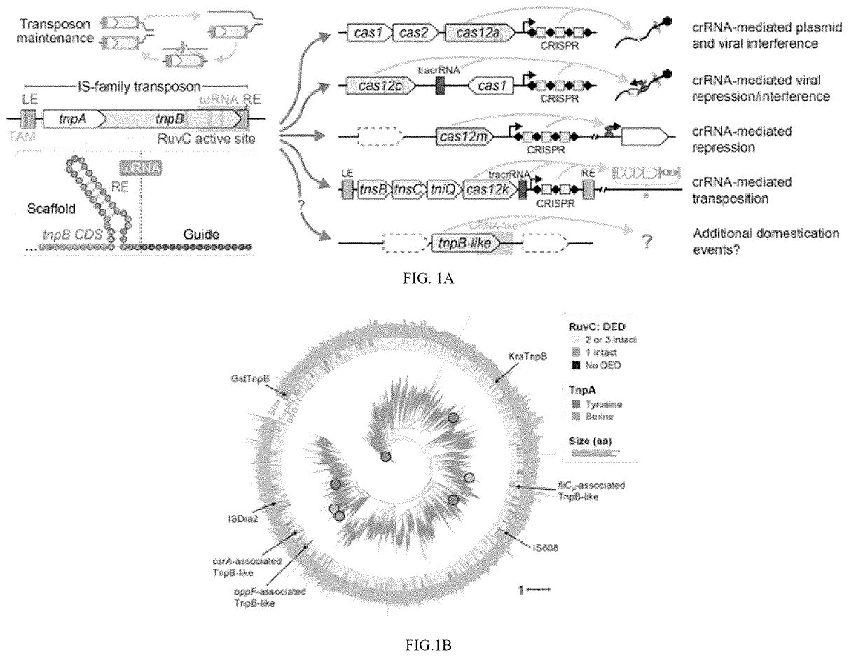

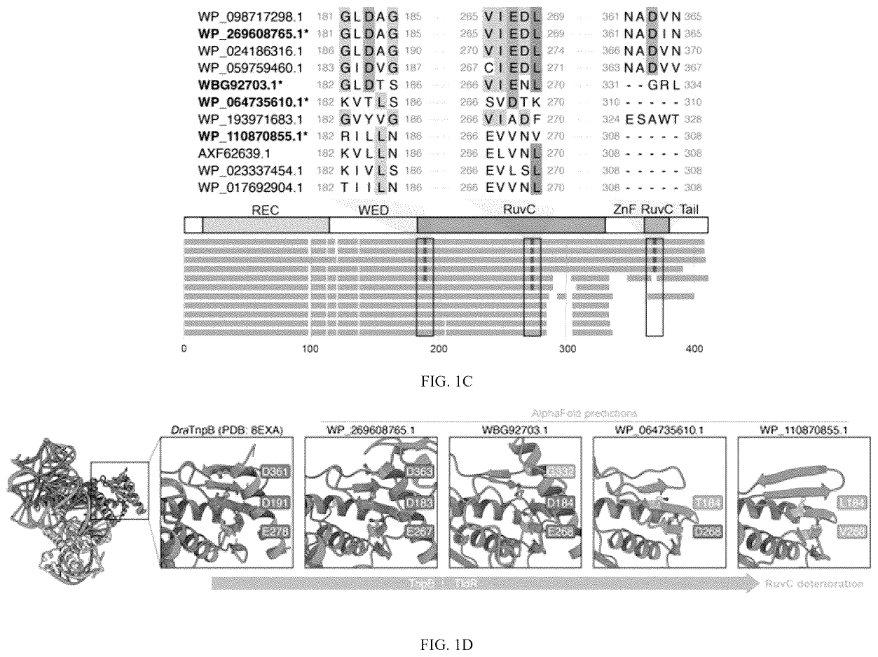

Transposon-encoded TnpB proteins represent a vast reservoir of RNA-guided nucleases that are found in association with diverse transposons/transposases across all three domains of life. In bacteria, tpB genes are encoded within IS200/IS605- and IS607-family transposons, which are minimal selfish genetic elements that are mobilized by a TnpA-family transposase but often exist in a non-autonomous form. These transposons harbor conserved left end (LE) and right end (RE) sequences that define the boundaries of the mobile DNA, and in addition to protein-coding genes, they also encode non-coding RNAs, referred to as ωRNA (or reRNA), that feature a scaffold region spanning the transposon RE and a ˜16-nt guide derived from the transposon-flanking sequence (FIG. 1A). It was recently demonstrated that TnpA-mediated transposition generates a scarless excision product at the donor site that is rapidly recognized and cleaved by TnpB-ωRNA complexes, in a reaction dependent on RNA-DNA complementarity and the presence of a cognate transposon/target-adjacent motif (TAM), leading to transposon reinstallation via DSB-mediated homologous recombination.

TnpB nucleases have been independently domesticated numerous times over evolutionary timescales, leading to the emergence of dozens of unique CRISPR-Cas12 subtypes that feature diverse guide RNA requirements and PAM specificities. In nearly all cases, Cas12 homologs rely on the same RuvC nuclease domain as TnpB for target cleavage, highlighting its conserved role in nucleic acid chemistry. However, recent studies uncovered atypical Cas12 homolog, Cas12c and Cas12m, that have lost the ability to cleave target DNA but instead bind and repress gene transcription as an alternative mechanism to preventing MGE proliferation. Type V-K CASTs similarly rely on nuclease-inactivated Cas12k homologs that are still active for RNA-guided DNA binding, leading to programmable transposition (FIG. 1A).

Disclosed herein is a family of TnpB-like nuclease-dead repressors (hereinafter TldR) that function not for transposition, but for RNA-guided transcriptional control, thus rendering the name “TnpB (transposase B)” inapposite. Using a custom bioinformatics pipeline, multiple independent TldR clades that evolved from transposon-encoded TnpB nucleases via RuvC active site deterioration, coincident with newly acquired, non-transposase gene associations, were identified. TldRs function with adjacently encoded non-coding guide RNAs (gRNAs) to target complementary DNA sequences flanked by a TAM within promoter regions, and target binding down-regulates gene expression through competitive exclusion of RNA polymerase.

These TldRs, Cas12 homologs, and conjugates thereof represent promising new reagents for genome engineering applications. While TldRs themselves are capable of repressing RNA expression, experiments utilizing TldR fused to effector polypeptides reveal the potential for augmented TldRs function. Thus, by tethering effector polypeptides to either the N- or C-terminus of a TldR or Cas12 homolog, or internally within the polypeptide, a variety of novel genome engineering tools are accessible, including but not limited to transcriptional activation tools (CRISPRa), transcriptional repression tools (CRISPRi), base editing tools (CBE and ABE), chromosomal locus imaging tools, prime editing reagents via fusion to reverse transcriptase domains, and additional epigenome reagents via fusion to domains that perform histone modifications, DNA modifications, or a combination thereof.

Provided herein are TldR proteins comprising one or more amino acid sequences having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1-508 and 1768-5926. In some embodiments, the TldR proteins comprise an amino acid sequence as shown in the Table below or Table 5. In some embodiments, the TldR proteins comprise an amino acid sequence of any of SEQ ID NOs: 1-508 and 1768-5926.

Also disclosed herein are catalytically inactive Cas12f (dCas12f) or Cas12f-like (dCas12f-like) proteins. Cas12f is a structurally determined ortholog of TnpB, such that the dCas12f and or dCas12f-like proteins share common ancestors (e.g., TnpB nucleases) with the TldR proteins. Similar to the TldR proteins, these dCas12f or dCas12f-like proteins and conjugates thereof represent promising new reagents for genome engineering applications.

Provided herein are dCas12f or dCas12f-like proteins comprising one or more amino acid sequences having at least 70% (e.g., having at least 75%, at least 80%, at least 85%, at least 90%, at least 92%, at least 95%, at least 96%, at least 97%, at least 98%, or at least 99%) identity to any of SEQ ID NOs: 6026-6042. In some embodiments, the dCas12f or dCas12f-like proteins comprise an amino acid sequence having at least 70% identity to any sequence in Table 7. In some embodiments, the dCas12f or dCas12f-like proteins comprise an amino acid sequences of any of SEQ ID NOs: 6026-6042.

Any of the proteins described or referenced herein may be fused or linked to at least one (e.g., 1, 2, 3, 4, 5, 6, 7, or more) effector polypeptides. Accordingly, also provided herein are protein conjugates comprising a TldR protein and at least one effector polypeptide. The TldR protein or dCas12f or dCas12f-like protein can be linked to effector polypeptide using standard chemical or enzymatic conjugation techniques. The protein conjugate can also be produced as a contiguous protein (e.g., a fusion protein) using genetic engineering techniques. The fusion protein can be expressed and purified as a single contiguous protein containing both the TldR protein or dCas12f or dCas12f-like protein and the effector polypeptide.

In the protein conjugate, the TldR protein or dCas12f or dCas12f-like protein and the effector polypeptide can be linked in any orientation (e.g., N-terminus to C-terminus or either terminus to an internal site) at any location as long as both can separately function and/or interact with their proposed targets. As such, the TldR protein or dCas12f or dCas12f-like protein conjugate described herein is not limited by the method, location, or orientation of the conjugation.

Effector polypeptides include proteins or protein domains that have additional functionality or activity useful to target certain DNA sequences. The effector polypeptide may comprise a number of functionalities, including but not limited to, nuclease function, recombinase function, epigenetic modifying function, transposase function, integrase function, resolvase function, invertase function, protease function, DNA methyltransferase function, DNA demethylase function, histone acetylase function, histone deacetylase function, transcriptional repressor function, transcriptional activator function, DNA binding protein function, transcription factor recruiting protein function, nuclear-localization signal function, DNA editing function (e.g., deaminase) or any combination thereof. For example, some effector domains function in transcriptional regulation via their ability to interact with the basal transcriptional machinery and general co-activators, interact with other transcription factors to allow cooperative binding, and/or directly or indirectly recruit histone and chromatin modifying enzymes.

In some embodiments, the TldR proteins or dCas12f or dCas12f-like proteins and conjugates thereof described herein are used to modulate gene regulatory activity, such as transcriptional or translational activity. For example, the at least one effector polypeptide may comprise activator and/or repressor activity that can affect transcription upstream and downstream of coding regions, and can be used to activate or repress gene expression. In some embodiments, the at least one effector polypeptide may include domains from transcription factors (activators, repressors, coactivators, co-repressors), silencers, and/or chromatin associated proteins and their modifiers (e.g., methylases, demethylases, acetylases and deacetylases).

Accordingly, in some embodiments, a TldR protein or dCas12f or dCas12f-like protein or conjugate thereof having a transcription activator effector polypeptide can be used to directly increase gene expression. In some embodiments, a TldR protein or dCas12f or dCas12f-like protein or conjugate thereof as disclosed herein comprising a transcriptional protein recruiting domain, or active fragment thereof, can be used to recruit transcriptional activators or repressors to a specific nucleic acid sequence to localize activators and repressors to modulate gene expression in a targeted manner.

In some embodiments, the effector polypeptide comprises transcriptional repressor function. Transcription repressors prevent, partially or completely, the transcription of genes near to their target site. Exemplary transcriptional repressors include, but are not limited to, KRAB-domain containing proteins, SID, and Sp1.

In some embodiments, the effector polypeptide comprises transcriptional activator function. Transcriptional activators can be generally defined as proteins, or domains thereof, that bind to specific sites on promoter DNA and bring about increased transcription of specific genes through interactions with other proteins. Exemplary transcriptional activators include, but are not limited to, VP64, p65, p53, c-Myb, GATA-1, EKLF, MyoD, E2F, dTCF, Tat, HSF1, RTA and SET7/9.

In some embodiments, the effector polypeptide comprises DNA methyltransferase or DNA methylase function. DNA methyltransferases (DNMT's) are a family of DNA modifying proteins composed of different isomers (e.g., DNMT1, DNMT3A, and DNMT3B). Other exemplary DNA methyltransferases include SssI methylase, Alul methylase, HaeIII methylase, Hhal methylase, and Hpall methylase. Their main mechanism of action is addition of a methyl group to the fifth carbon of a cytosine residue (5mc) located adjacent to a guanine residue.

In some embodiments, the effector polypeptide comprises DNA demethylase function. DNA demethylation can be mediated by at least three enzyme families: (i) the ten-eleven translocation (TET) family, mediating the conversion of 5mC into 5hmC; (ii) the AID/APOBEC family, acting as mediators of 5mC or 5hmC deamination; and (iii) the BER (base excision repair) glycosylase family involved in DNA repair.

Kinases, phosphatases, and other proteins that modify or regulate other polypeptides involved in gene regulation are also useful as effector polypeptides. Such modifiers are often involved in switching on or off transcription mediated by, for example, hormones. Other useful domains for regulating gene expression can also be obtained from the gene products of oncogenes (e.g., myc, jun, fos, myb, max, mad, rel, ets, bcl, myb, mos family members) and their associated factors and modifiers.

The effector polypeptide can be used to target enzymatic activity to locations containing the target nucleic acid sequence to which the gRNA is directed. For example, in some embodiments, effector polypeptides having integrase or transposase activity can be used to promote integration of exogenous nucleic acid sequence into specific nucleic acid sequence regions and/or eliminate (knock-out) specific endogenous nucleic acid sequence.

Integrases allow for the insertion of nucleic acids, for example, into a host genome (mammalian, human, mouse, rat, monkey, frog, fish, plant (including crop plants and experimental plants like Arabidopsis), laboratory or biomedical cell lines or primary cell cultures, C. elegans, fly (Drosophila), etc.). Integrases are found in a retrovirus such as HIV (human immunodeficiency virus) and lambda integrase.

In some embodiments, the effector polypeptide comprises transposase functionality. Transposases are enzymes that bind to the end of a transposon and catalyze its movement by a cut and paste mechanism or a replicative transposition mechanism. Exemplary transpoases include, but are not limited to, Tc1 transposase, Mos1 transposase, Tn5 transposase, and Mu transposase

In some embodiments, the effector polypeptide modifies epigenetic signals and thereby modifies gene regulation, for example by promoting histone acetylase and histone deacetylase activity. The term “epigenetic modifier,” as used herein, refers to a protein or catalytic domain thereof having enzymatic activity that results in the epigenetic modification of DNA, for example, chromosomal DNA. Epigenetic modifications include, but are not limited to, histone modifications including methylation and demethylation (e.g., mono-, di- and tri-methylation), histone acetylation and deacetylation, as well as histone ubiquitylation, phosphorylation, and sumoylation.

Histone acetylation and deacetylation are the processes by which the lysine residues within the N-terminal tail protruding from the histone core of the nucleosome are acetylated and deacetylated as part of gene regulation. These reactions are typically catalyzed by enzymes with histone acetyltransferase (HAT) or histone deacetylase (HDAC) activity. Histone acetyltransferases include GNAT family proteins (e.g., Gcn5, Gcn5L, p300/CREB-binding protein associated factor (PCAF), Elp3, HPA2 and HAT1) and MYST family proteins (e.g., Sas3, essential SAS-related acetyltransferase (Esa1), Sas2, Tip60, MOF, MOZ, MORF, and HBO1). Histone deacetylases fall into four classes. Class I includes HDACs 1, 2, 3, and 8. Class II is divided into two subgroups, Class IIA and Class IIB. Class IIA includes HDACs 4, 5, 7, and 9 while Class IIB includes HDACs 6 and 10. Class III contains the Sirtuins and Class IV contains only HDAC11. Classes of HDAC proteins are divided and grouped together based on the comparison to the sequence homologies of Rpd3, Hos1 and Hos2 for Class I HDACs, HDAI and Hos3 for the Class II HDACs and the sirtuins for Class III HDACs.

The site-specific methylation and demethylation of histone residues are catalyzed by methyltransferases and demethylases, respectively. Histone methylases transfer methyl groups to amino acids (e.g., lysine and arginine) of histone proteins, ultimately effecting transcription of genes. Methylases include SET1, MLL, SMYD3, G9a, GLP, EZH2, and SETDB1. Histone demethylases catalyze the removal of methyl marks from histones, an activity associated with transcriptional regulation and DNA damage repair. Demethylases include, for example, KDMIA, KDMIB, KDM2A, KDM2B, UTX, UTY, Jumonji C (JmJC) domain-containing demethylases, and GSK-J4.

In some embodiments, the effector polypeptide comprises nuclease activity. A nuclease is an agent that induces a break in a nucleic acid sequence, e.g., a single or a double strand break in a double-stranded DNA sequence. Nucleases include those which cut at or near a preselected or specific sequence and those which are not site specific. For example, nucleases include, but are not limited to, zinc finger nucleases (ZFN), homing endonucleases, meganucleases, restriction enzymes, TAL effector nucleases, Argonaute nucleases, CRISPR nucleases, comprising, for example, Cas9, Cpf1, Csm1, CasX or CasY nucleases, micrococcal nuclease, staphylococcal nuclease, DNase I, T7 endonuclease, or catalytically active fragments thereof.

In some embodiments, the effector polypeptide comprises invertase activity. Invertase activity can be used to alter genome structure by swapping the orientation of a DNA fragment.

In some embodiments, the effector polypeptide comprises recombinase activity. A recombinase is a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3 (also known as TnpR), β-six, CinH, ParA, γδ, Bxb1, φC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange.

In some embodiments, the effector polypeptide comprises resolvase activity. Resolvases are site-specific recombinases that function to excise (as a circle) a segment of DNA contained between two recombination sites (called res) and include, for example, Ruv C resolvase, Holiday junction resolvase Hjc, Tn3 and γδ resolvase.

In some embodiments, the effector polypeptide comprises a peptide or polypeptide sequence responsive to a ligand, such as a hormone receptor ligand binding domain, including, for example, the ligand binding domains of the estrogen receptor, the glucocorticosteroid receptor, and the like. Such effector domains can be used to act as “gene switches,” and be regulated by inducers, such as small molecule or protein ligands, specific for the ligand binding domain.

In some embodiments, the effector polypeptide comprises sequences or domains of polypeptides that mediate direct or indirect protein-protein interactions, including, for example, a leucine zipper domain, a STAT protein N terminal domain, and/or an FK506 binding protein.

In some embodiments, the effector polypeptide comprises DNA editing function (e.g., deaminase, DNA repair activity, DNA damage activity, dismutase activity, alkylation activity, depurination activity, oxidation activity, pyrimidine dimer forming activity, polymerase activity (e.g., reverse transcriptase), ligase activity, helicase activity, photolyase activity or glycosylase activity).

In some embodiments, the effector polypeptide comprises a deaminase, or functional fragment thereof. The deaminase, or functional fragment thereof may be derived from a naturally occurring deaminase or variant thereof (e.g., a protein, enzyme, or domain with an amino acid sequence having at least 70% identity to a naturally occurring deaminase). Alternatively, the deaminase may be a synthetic or engineered deaminase. In some embodiments, the deaminase, or functional fragment thereof, is an adenosine deaminase, also sometimes referred to as an adenine deaminase. In some embodiments, the adenosine deaminase is derived from a bacterium, such as, E. coli. In some embodiments, the deaminase, or functional fragment thereof, is a cytidine deaminase.

In some embodiments, the activity mediated by the effector polypeptide is a non-biological activity, such as a fluorescence activity (e.g., fluorescent proteins), luminescence activity (e.g., a luminescent protein or enzyme which results in luminescence when interacting with a substrate (e.g., luciferase)), or binding activity, such as those mediated by maltose binding protein (“MBP”), glutathione S transferase (GST), hexahistidine, c-myc, and the FLAG epitope, for facilitating detection, purification, monitoring expression, and/or monitoring cellular and subcellular localization of the polypeptide to which the effector domain is appended. In such embodiments, the systems can also be used as a diagnostic reagent, for example, to detect mutations in gene sequences, to purify restriction fragments from a solution, or to visualize DNA fragments of a gel.

The effector polypeptides described herein are illustrative and merely provide the skilled artisan with examples of effectors that can be used in combination with the TldR proteins or dCas12f or dCas12f-like protein or conjugates thereof described herein.

In some embodiments, the effector polypeptide comprises a transcription activator, a transcription repressor, a base editor, an epigenetic modifier, a chromosomal locus imaging agent (e.g., fluorescent protein or protein tag), or a combination thereof.

In some embodiments, the effector polypeptide comprises fragments of proteins that have been separated from their natural DNA binding domains and engineered to be part of a fusion protein with the protein described herein. In some embodiments, the effector polypeptides are proteins which normally bind to other proteins or factors which result in their recruitment to a specific or non-specific nucleic acid.

Also provided herein are TnpB-transposase fusion proteins comprising one or more amino acid sequences disclosed in the Table provided elsewhere herein. In some embodiments, the TnpB-transposase fusion proteins comprise one or more amino acid sequences having at least 70% (e.g., having at least 75%, at least 80%, at least 85%, at least 90%, at least 92%, at least 95%, at least 96%, at least 97%, at least 98%, or at least 99%) identity to any of SEQ ID NOs: 1453-1539. In some embodiments, the TnpB-transposase fusion proteins comprise an amino acid sequences of any of SEQ ID NOs: 1453-1539.

Any of the proteins described or referenced herein may comprise one or more amino acid substitutions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

Any of the proteins disclosed herein may further comprise one or more proteins, polypeptides (e.g., protein domain sequences), or peptides fused or linked to the polypeptide. Accordingly, also provided herein are protein conjugates comprising a TldR protein or a dCas12f or dCas12f-like protein. The one or more proteins, polypeptides (e.g., protein domain sequences), or peptides may be appended at an N-terminus, a C-terminus, internally, or a combination thereof. The one or more proteins, polypeptides (e.g., protein domain sequences), or peptides may be fused or linked in any orientation in relationship to the disclosed protein. For example, the proteins disclosed herein may be fused or linked to another protein or protein domain that provides for tagging or visualization (e.g., GFP).

Any of the proteins or conjugates described or referenced herein may further have a nuclear localization sequence (NLS). The at least one nuclear localization sequence may be appended to the N-terminus, the C-terminus, or embedded in the protein (e.g., inserted internally within the open reading frame (ORF)). The proteins or conjugates s may comprise one or more nuclear localization sequences. The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprises a single cluster of positively charged or basic amino acids. In some embodiments, the monopartite NLS comprises a sequence of K-K/R—X-K/R, wherein X can be any amino acid. Exemplary monopartite NLSs include, without limitation, those from the SV40 large T-antigen (PKKKRKVEDP; SEQ ID NO: 6164), c-Myc (PAAKRVKLD; SEQ ID NO: 6165), and TUS-proteins (Kaczmarczyk S J et al. PLOS ONE 5 (1): e8889.2010). In select embodiments, the NLS comprises a c-Myc NLS.

In some embodiments, the NLS is a bipartite sequence. Bipartite NLSs comprise two clusters of basic amino acids, separated by a spacer of about 9-12 amino acids. Exemplary bipartite NLSs include the NLS of nucleoplasmin, KR [PAATKKAGQA]KKKK (SEQ ID NO: 6166), the NLS of EGL-13, MSRRRKANPTKLSENAKKLAKEVEN (SEQ ID NO: 6167), the bipartite SV40 NLS, KRTADGSEFESPKKKRKV (SEQ ID NO: 6168).

Any of the proteins or conjugates described or referenced herein may further have an epitope tag (e.g., 3×FLAG tag, an HA tag, a Myc tag, and the like). The epitope tags may be at the N-terminus, a C-terminus, or a combination thereof of the corresponding protein. In some embodiments, the epitope tag may be adjacent, either upstream or downstream, to a nuclear localization sequence.

The effector polypeptide, NLS, or epitope tag may be appended to the proteins described herein by a linker. The linker may have any of a variety of amino acid sequences. Suitable linkers include polypeptides of between 1 amino acids and 100 amino acids in length, between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the protein. Peptide linkers with a degree of flexibility can be used. The linking peptides may have virtually any amino acid sequence, bearing in mind that the preferred linkers will have a sequence that results in a generally flexible peptide. Small amino acids, such as glycine and alanine, are generally used in creating a flexible peptide. A variety of different linkers are commercially available and are considered suitable for use, including but not limited to, glycine-serine polymers, glycine-alanine polymers, and alanine-serine polymers.

Compositions comprising the TldR proteins or conjugates thereof, dCas12f or dCas12f-like protein or conjugates thereof, or TnpB-transposase fusion proteins, as described herein or a nucleic acid molecule comprising a sequence encoding the TldR proteins or conjugates thereof, dCas12f or dCas12f-like protein or conjugates thereof, or TnpB-transposase fusion proteins, are also provided.

### Systems

Further provided herein are systems for modifying a target nucleic acid sequence.

In some embodiments, the systems comprise: a TldR protein or a conjugate thereof, a dCas12f or dCas12f-like protein or a conjugate thereof, or a TnpB-transposase fusion protein, as described herein and/or one or more nucleic acids encoding thereof; and optionally, at least one guide RNA, or one or more nucleic acids encoding thereof, complementary to at least a portion of a target nucleic acid.

The gRNA or portion thereof that hybridizes to the target nucleic acid (a target site) may be any length. In some embodiments, the gRNA sequence that hybridizes to the target nucleic acid is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid.

To facilitate gRNA design, many computational tools have been developed (See Prykhozhij et al. (PLOS ONE, 10 (3): (2015)); Zhu et al. (PLOS ONE, 9 (9) (2014)); Xiao et al. (Bioinformatics. Jan. 21 (2014)); Heigwer et al. (Nat Methods, 11 (2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence. Alternatively, the gRNA and scaffold sequence may be provided as omega RNA (ωRNA). Exemplary ωRNAs are provided in the Tables herein.

The gRNA may be a non-naturally occurring gRNA.

The system may further comprise a target nucleic acid. The terms “target sequence,” “target nucleic acid,” and “target site” (e.g., a “target genomic DNA sequence”) are used interchangeably herein to refer to a polynucleotide (nucleic acid, gene, chromosome, genome, etc.) to which a guide sequence (e.g., a synthetic guide RNA) is designed to have complementarity, wherein hybridization between the target sequence and a guide sequence promotes the formation of a complex, e.g., of the guide RNA, target, and TldR protein, or a conjugate thereof, a dCas12f or dCas12f-like protein or conjugate thereof, or a TnpB-transposase fusion protein provided sufficient conditions for binding exist. The target sequence and guide sequence need not exhibit complete complementarity, provided that there is sufficient complementarity to cause hybridization and promote formation of the complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art.

The target nucleic acid may or may not be flanked by a transposon adjacent motif (TAM). A TAM can be upstream of the target sequence. In one embodiment, the target sequence is immediately flanked on the 5′end by a TAM sequence. A TAM can be 1, 2, 3, 4, 5, 6, 7, 8, 9, 10 or more nucleotides in length. In certain embodiments, a TAM is between 2-6 nucleotides in length. In some embodiments, the TAM comprises a sequence of TT (C/T) A (A/T/C). In select embodiments, the TAM sequence is TTTAT or TTCAT. In some embodiments, the TAM sequence comprises TGG. Exemplary TAM sequences are provided in the Examples herein. There may be mismatches distal from the TAM.

However, structure-guided mutations and directed evolution experiments have been successfully utilized to modify the targeting constraints of other RNA-guided nucleases (e.g., modification of PAM requirements in Cas9/Cas12 CRISPR-based systems). In other embodiments, TldR proteins, dCas12f or dCas12f-like proteins, or TnpB-transposase fusion proteins with modified TAM-interacting residues are used, in conjunction with any of the above stated embodiments, to extend the range of genomic targets.

The system may further include a donor nucleic acid. The donor nucleic acid may be a part of a bacterial plasmid, bacteriophage, a virus, autonomously replicating extra chromosomal DNA element, linear plasmid, linear DNA, linear covalently closed DNA, mitochondrial or other organellar DNA, chromosomal DNA, and the like. In some embodiments, the donor nucleic acid comprises a cargo nucleic acid sequence.

The donor nucleic acid may be flanked by at least one transposon end sequence. In some embodiments, the donor nucleic acid is flanked on the 5′ and the 3′ end with a transposon end sequence. The term “transposon end sequence” refers to any nucleic acid comprising a sequence capable of forming a complex with the transposase enzymes thus designating the nucleic acid between the two ends for rearrangement. Usually, these sequences contain inverted repeats and may be about 10-150 base pairs long, however the exact sequence requirements differ for the specific transposase enzymes. Transposon end sequences are well known in the art. Transposon ends sequences may or may not include additional sequences that promotes or augment transposition.

The donor nucleic acid, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or greater.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell (e.g., a cell of a non-human primate or a human cell). Thus, in some embodiments, disclosed herein are systems for nucleic acid modification of a target nucleic acid sequence in a eukaryotic cell (e.g., a mammalian cell, a human cell).

### Nucleic Acids

The one or more nucleic acids encoding a TldR protein, or conjugate thereof, a dCas12f or dCas12f-like protein or a conjugate thereof, or a TnpB-transposase fusion protein, and guide RNA (e.g., ωRNA) may be any nucleic acid including DNA, RNA, or combinations thereof. In some embodiments, nucleic acids comprise one or more messenger RNAs, one or more vectors, or any combination thereof.

[0.145] In certain embodiments, engineering the system for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., about 65%, about 70%, about 75%, about 80%, about 85%, about 90%, about 95%, or about 98%) of the codons encoded therein are mammalian preferred codons.

The present disclosure also provides for DNA segments encoding the proteins and nucleic acids disclosed herein, vectors containing these segments and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the system may be removed from the cells under certain conditions. For example. this may allow for DNA integration by transforming bacteria of interest, but then being left with engineered strains that have no memory of the plasmids or vectors used for the integration.

Drug selection strategies may be adopted for positively selecting for cells that underwent DNA integration. A donor nucleic acid may contain one or more drug-selectable markers within the cargo. Then presuming that the original donor plasmid is removed, drug selection may be used to enrich for integrated clones. Colony screenings may be used to isolate clonal events.

A variety of viral constructs may be used to deliver the present system or components thereof (such as a TldR protein, or conjugate thereof, a dCas12f or dCas12f-like protein or a conjugate thereof, or a TnpB-transposase fusion protein, and gRNA) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7 (1): 33-40; and Walther W. and Stein U., 2000 Drugs, 60 (2): 249-71, incorporated herein by reference.

In one embodiment, a DNA segment encoding a TldR protein, or conjugate thereof, a dCas12f or dCas12f-like protein or a conjugate thereof, or a TnpB-transposase fusion protein, and/or a guide RNA (e.g., ωRNA) is contained in a plasmid vector that allows expression of the protein(s) and subsequent isolation and purification produced by the recombinant vector. Accordingly, the proteins disclosed herein can be purified following expression, obtained by chemical synthesis, or obtained by recombinant methods.

To construct cells that express the present system or components thereof, expression vectors for stable or transient expression may be constructed via conventional methods as described herein and introduced into cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. The system may be used with various bacterial hosts.

[01.56] In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EFla (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColEl for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRPI genes of S. cerevisiae.

When introduced into the cell, the vectors may be maintained as an autonomously replicating sequence or extrachromosomal element or may be integrated into host DNA.

In one embodiment, the present disclosure comprises integration of exogenous DNA into an endogenous gene. Alternatively, an exogenous DNA is not integrated into the endogenous gene. The DNA may be packaged into an extrachromosomal or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, donor polynucleotides and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see, e.g., Sharei et al. Proc. Natl. Acad. Sci. USA (2013) 110 (6): 2082-2087, incorporated herein by reference); or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83), incorporated herein by reference.

### Methods

Also disclosed herein are methods for nucleic acid modification or integration utilizing the disclosed polypeptides, nucleic acids encoding thereof, systems, or kits.

The methods may comprise contacting a target nucleic acid sequence with a system, a polypeptide, a nucleic acid, or a composition disclosed herein. The descriptions and embodiments provided above for the system, the polypeptide, the gRNA (e.g., ωRNA), and the nucleic acids are applicable to the methods described herein.

The phrase “modifying a nucleic acid sequence” or “nucleic acid modification” as used herein, refers to modifying at least one physical feature of a nucleic acid sequence of interest. Nucleic acid modifications include, for example, single or double strand breaks, deletion, or insertion of one or more nucleotides, and other modifications that affect the structural integrity or nucleotide sequence of the nucleic acid sequence. In some embodiments, the modifications may include cleavage of the target nucleic acid, excision of the target nucleic acid, integration of the donor nucleic acid, or a combination thereof. Modifying a nucleic acid sequence may further encompass any or all of the functions provided by the effector polypeptide as described above.

The target nucleic acid sequence may be in a cell. In some embodiments, contacting a target nucleic acid sequence comprises introducing the system into the cell. As described above the system may be introduced into eukaryotic or prokaryotic cells by methods known in the art. In some embodiments, the cell is a mammalian cell. In some embodiments, the cell is a human cell.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target nucleic acid sequence encodes a protein or polypeptide.

Polynucleotides containing the target nucleic acid sequence may include, but is not limited to, purified chromosomal DNA, total cDNA, cDNA fractionated according to tissue or expression state (e.g., after heat shock or after cytokine treatment other treatment) or expression time (after any such treatment) or developmental stage, plasmid, cosmid, BAC, YAC, phage library, etc. Polynucleotides containing the target site may include DNA from organisms such as Homo sapiens, Mus domesticus, Mus spretus, Canis domesticus, Bos, Caenorhabditis elegans, Plasmodium falciparum, Plasmodium vivax, Onchocerca volvulus, Brugia malayi, Dirofilaria immitis, Leishmania, Zea maize, Arabidopsis thaliana, Glycine max, Drosophila melanogaster, Saccharomyces cerevisiae, Schizosaccharomyces pombe, Neurospora, Escherichia coli, Salmonella typhimurium, Bacillus subtilis, Neisseria gonorrhoeae, Staphylococcus aureus, Streptococcus pneumonia, Mycobacterium tuberculosis, Aquifex, Thermus aquaticus, Pyrococcus furiosus, Thermus littoralis, Methanobacterium thermoautotrophicum, Sulfolobus caldoaceticus, and others.

The methods may comprise administering to the subject, in vivo, or by transplantation of ex vivo treated cells, an effective amount of the described system. In some embodiments, the vector(s) is delivered to the tissue of interest by, for example, an intramuscular, intravenous, transdermal, intranasal, oral, mucosal, or other delivery methods.

The components of the present system or ex vivo treated cells may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful DNA modification or integration is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The disclosed methods may modify a target DNA sequence in a cell so as to modulate expression of the target DNA sequence, e.g., expression of the target DNA sequence is increased, decreased, or completely eliminated (e.g., via deletion of a gene). The modifications of the target sequence may lead to, for example, gene correction, gene replacement, gene tagging, transgene insertion, nucleotide deletion/addition/correction, gene disruption, gene mutation, gene knock-down, etc.

In some embodiments, the methods described herein may be used to correct one or more defects or mutations in a gene (referred to as “gene correction”). In such cases, the target sequence encodes a defective version of a gene, and the disclosed compositions and systems further comprise a donor nucleic acid molecule which encodes a wild-type or corrected version of the gene. Accordingly, in some embodiments, the methods described herein may be used to insert a gene or fragment thereof into a cell.

In another embodiment, the method of modifying a target sequence can be used to delete nucleic acids from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the methods described herein may be used to genetically modify a plant or plant cell. The present methods may be used with various microbial species, including human pathogens that are medically important, and bacterial pests that are key targets within the agricultural industry, as well as antibiotic resistant versions thereof. The present systems and methods may be used to inactivate microbial genes. In some embodiments, the gene is an antibiotic resistance gene. The methods described here also provide for treating a disease or condition in a subject. The methods may comprise administering to the subject, in vivo, or by transplantation of ex vivo treated cells (e.g., disclosed T cells), a therapeutically effective amount of the present system, polypeptides, or components thereof.

In some embodiments, the methods are used to treat a pathogen or parasite on or in a subject by altering the pathogen or parasite. In some embodiments, the methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1 (1): 192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (e.g., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene. The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

### Kits

Also within the scope of the present disclosure are kits that include the components of the present system, such as a TldR protein, or a conjugate thereof, a dCas12f or dCas12f-like protein or a conjugate thereof, or a TnpB-transposase fusion protein, a guide RNA (e.g., ωRNA), and/or a nucleic acid encoding thereof.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system. The device may include an infusion device, an intravenous solution bag, a hypodermic needle, a vial, and/or a syringe.

### EXAMPLES

The following are examples and are not to be construed as limiting.

### Example 1

Methods for Targeted DNA Modification Using Nuclease-Inactivated TnpB Homologs (dTnpB/TldR)

RNA-guided nucleases (e.g., Cas9, Cas12, IscB, and TnpB) are components of bacterial/archaeal immune systems or mobile genetic elements, that have been repurposed for genome modification. In particular, TnpB proteins are RNA-guided nucleases encoded in diverse insertion (e.g., IS200/IS605 superfamily) elements, and are ancestral to Cas12 CRISPR-RNA-guided nucleases (Meers, C. et al. bioRxiv 2023.03.14.532601 (2023) and references therein). Evolutionary offshoots of TnpB include naturally-occurring, nuclease dead Cas12 homologs that are capable of programmable DNA-cargo transposition (Cas12k from CRISPR-associated transposons, or CAST systems) and programmable repression of RNA transcription (Cas12m from type V-M CRISPR systems). While Cas12 proteins are large polypeptides, raising potential challenges in delivering these nucleases for therapeutic applications, TnpB proteins are compact effectors that may alleviate delivery size constraints. Here, naturally-occurring nuclease-inactivated TnpB proteins that direct RNA-guided DNA binding are identified and described, and serve as a new platform technology for the development of tools that include programmable transcriptional repression and activation, base editing, prime editing, epigenome editing, and other applications relying on RNA-guided DNA target binding and specification. These applications may occur in diverse cell types, including bacterial cells, plant cells, animal cells, human cells, and in in vivo contexts.

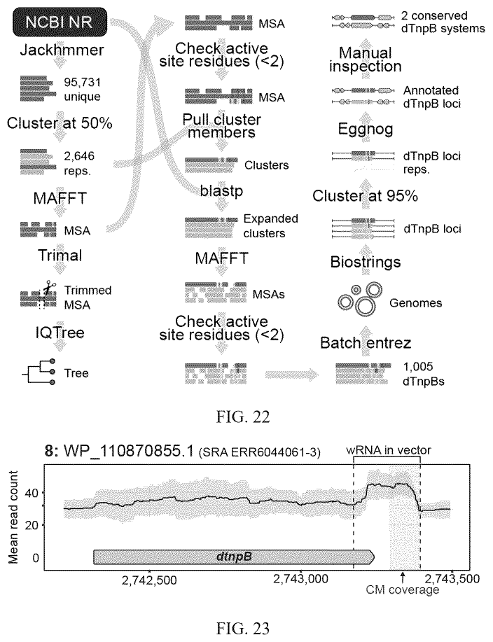

Bioinformatic identification of naturally deactivated nuclease-dead TnpB homologs A bioinformatic pipeline was developed to identify TnpB homologs with point mutations or C-terminal truncations that inactivate the RuvC nuclease domain (e.g., dTnpB) (FIG. 22). An initial search of the NCBI non-redundant (NR) protein database-queried with TnpB sequences from H. pylori and G. sterothermophilus (WP_078217163.1 and WP_047817673.1, respectively) in Jackhmmer-resulted in the identification of 95,731 unique TnpB-like proteins, that were further clustered at 50% amino acid identity (across 50% sequence coverage) to produce a set of 2,646 representative TnpB sequences. Multiple sequences alignments were then constructed to assess the conservation of RuvC catalytic residues in each TnpB protein sequence, using structurally determined orthologs (e.g., ISDra2 TnpB and Cas12f; PDB: 8HIJ and 5L48, respectively) as references.

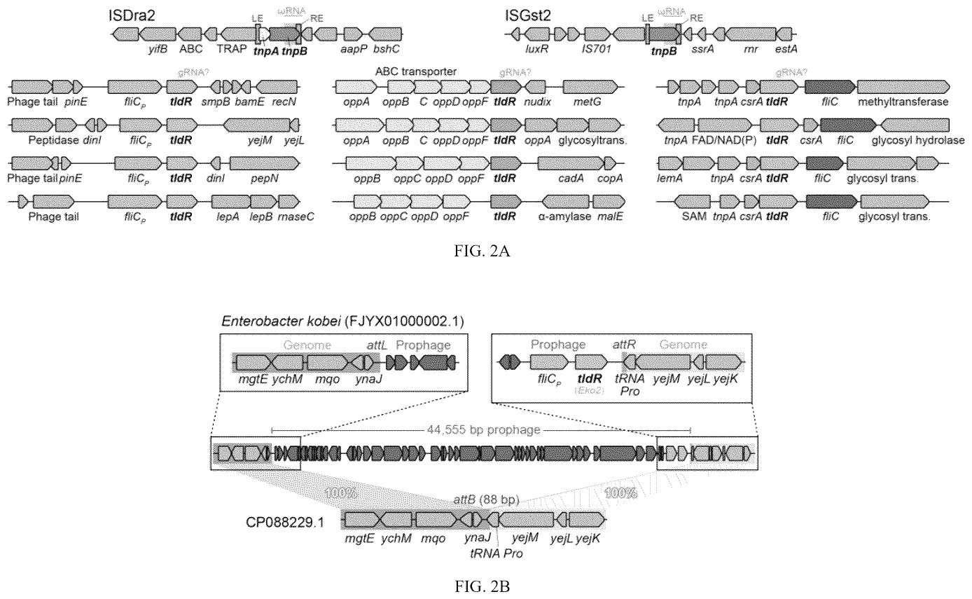

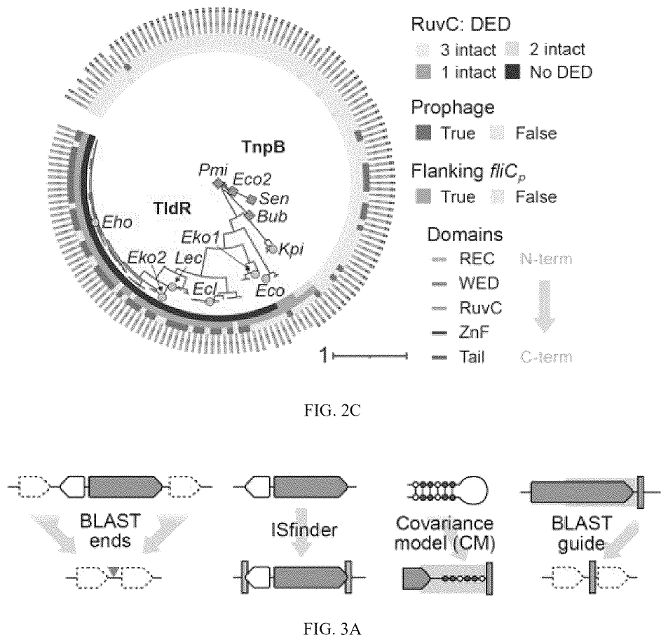

For sequences with less than two active site residues identified (e.g., dTnpB sequences), related homologs were retrieved from initial sequence clusters, and additional related homologs were identified via BLASTP searches of the NR protein database. This approach resulted in the identification of 8,889 dTnpB proteins (FIG. 22). Genomes encoding each dTnpB were retrieved from NCBI using the batch-entrez tool. dTnpB-encoding loci (e.g., dtnpB+/−20kpb) were extracted using the Biostrings package in R and were annotated with Eggnog. The initial alignment of TnpB/dTnpB representatives was used to construct a phylogenetic tree in IQTree, that guided manual investigation of dTnpB clades (FIG. 1B). Several stable genetic associations between dinpB and other genes (e.g., fliC or ABC transporter components) in different genetic contexts support the natural emergence of dTnpBs proteins for functions that do not require RuvC nuclease activity (e.g., transcriptional repression) (FIG. 2A). Structural predictions and multiple sequence alignments lend additional support to the gradual evolutionary loss of RuvC active site residues in dTnpBs (FIGS. 1C-1D), suggesting that selective pressures have led to their repurposing in natural contexts.

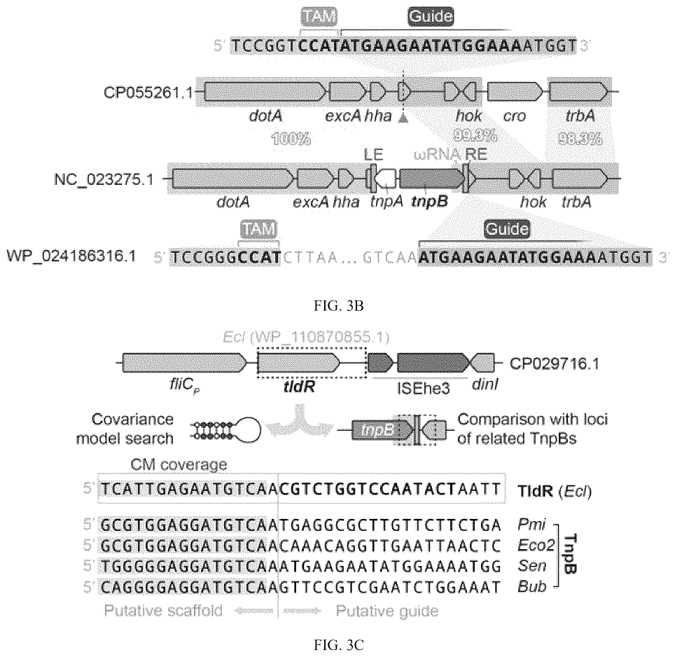

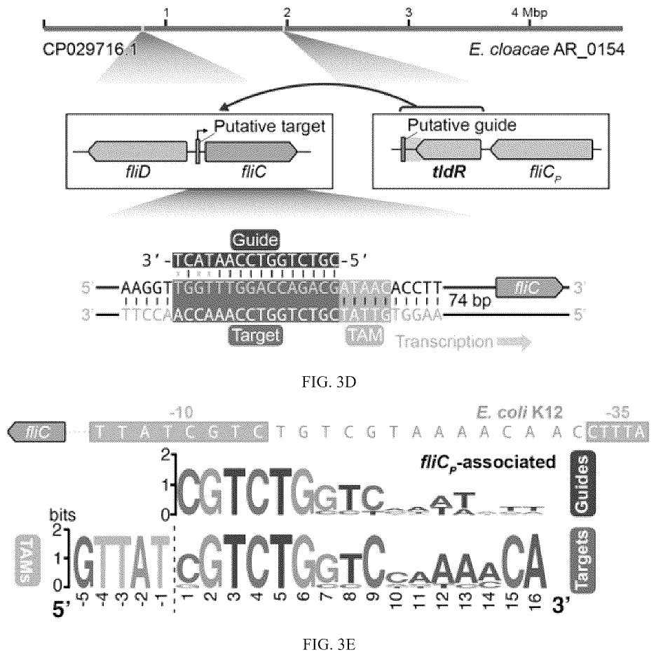

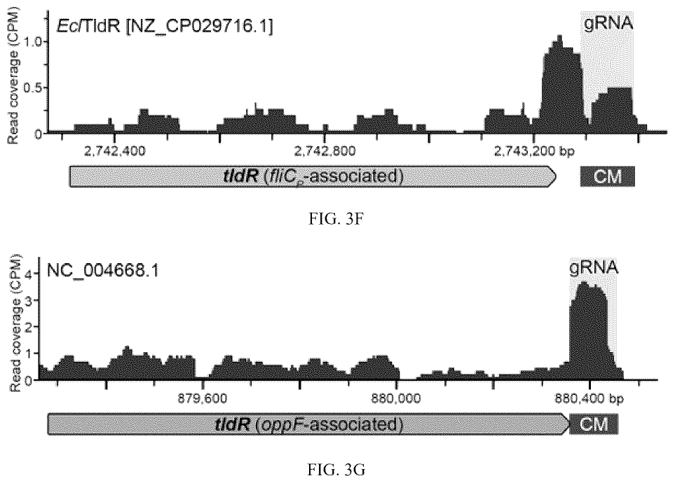

TnpB proteins utilize ωRNAs (OMEGA-RNAs) comprised of a scaffold and guide sequence to direct RuvC-mediated DNA cleavage. Analyses of publicly available RNAseq data indicates that transcription occurs beyond the 3′ end of dTnpB coding sequences, consistent with previous reports of TnpB ωRNA expression (FIGS. 3C and 23). To define the boundaries of ωRNA scaffolds in dTnpB-coding loci, sequence covariation models were utilized, described previously (Meers, C. et al. bioRxiv 2023.03.14.532601 (2023) doi: 10.1101/2023.03.14.532601). The CMsearch function of Infernal (Inference of RNA alignments) was used to scan nucleotide sequences of a subset of dTnpB loci and 500 basepair flanks, resulting in the identification of putative dTnpB ωRNA scaffold sequences (FIGS. 3C and 23 and sequences below). dTnpB ωRNA scaffold boundaries were confirmed by comparing dTnpB loci to ωRNAs from confidently predicted, catalytically active TnpB loci (FIGS. 3C and 23). Putative dTnpB guide sequences could then be retrieved from the 3′-boundary of putative ωRNA scaffolds, enabling prediction of native dTnpB targets (putative guides shown below). Homology between putative dTnpB guides and 5′-untranslated regions of protein coding genes indicates that dTnpBs have likely evolved to function as natural transcriptional repressors (FIG. 3D).

Utilization of dTnpB for genome targeting and modification applications dTnpB proteins represent a new and adaptable structural platform for programmable gene repression/activation, and genomic/epigenetic modification. While dTnpBs proteins themselves are capable of repressing RNA expression, experiments utilizing synthetically inactivated RNA-guided nucleases fused to transcriptional regulators reveal the potential for augmented dTnpB function. Thus, by tethering effector domains to either the N- or C-terminus of dTnpB, or internally within the dTnpB polypeptide, a variety of novel genome engineering tools are accessible.

In the paragraphs that follow, a series of embodiments are presented that describe new tools for transcriptional activation tools (CRISPRa), transcriptional repression tools (CRISPRi), base editing tools (CBE and ABE), and chromosomal locus imaging tools. Additional embodiments include the development of prime editing reagents via fusion to reverse transcriptase domains, and additional epigenome reagents via fusion to domains that perform histone modifications, DNA modifications, or a combination thereof.

In one embodiment, dTnpB proteins, together with appropriate nuclear localization signals (NLS), selectively bind to genomic target sites, resulting in transcriptional repression. Targeting is guided by the ωRNA.

dTnpB-based transcriptional activators are constructed by fusing activation domains, such as VP64, to the N-terminus or C-terminus of dTnpB, or internally within the dTnpB polypeptide, together with appropriate nuclear localization signals (NLS). In addition to the VP64-dTnpB fusions described, a range of other activation domains are used in other embodiments. The multi-valent recruitment of transcriptional activators to the target site, achieved by tethering multiple VP64 units via a polypeptide linker, leads to potent transcriptional activation in response to target with just a single ωRNA.

In other embodiments, dTnpB may be fused to a wide range of alternative activation or epigenome modification domains. An NLS is included, and may be encoded at the N-terminus, C-terminus, or internally. dTnpB selectively binds to genomic target sites, resulting in activity of the fused effector domains.

In other embodiments, dTnpB is fused to transcriptional repression domains, such as KRAB domains or other repressive domains. An NLS is included, and may be encoded at the N-terminus, C-terminus, or internally. dTnpB selectively binds to genomic target sites, resulting in activity of the fused effector domains.

In other embodiments, dTnpB is fused to fluorescent proteins (FPs), such as GFP, for chromosomal labeling. An NLS is included, and may be encoded at the N-terminus, C-terminus or internally. dTnpB selectively binds to genomic target sites, along with one or multiple copies of a FP tethered by a polypeptide linker, such that the high valency leads to high signal-to-noise localization of one or multiple chromophores at the same target site, in response to targeting by just one ωRNA.

In other embodiments, dTnpB is fused to base editing reagents, as described (Anzalone et al., Nat Biotechnol 38, 824-844 (2020) and references therein). Various fusions enable variable windows of base editing across guide-target duplex and untargeted strand. In the case of cytosine base editors (CBEs), the target dTnpB component is fused to both the deaminase domain as well as uracil glycosylase inhibitor domains. In the case of adenine base editors (ABEs), the target dTnpB component is fused to two tandem TadA domains, one of which is evolved to deaminate deoxyadenosine. dTnpB base editors may also be combined with Cas9 nickase enzymes, in order to nick one strand of DNA and thereby improve purity of the final product.

Typical TnpB guide sequences are 12-16 basepairs in length, and utilize a target-adjacent motif (TAM) for target binding. However, structure-guided mutations and directed evolution experiments have been successfully utilized to modify the targeting constraints of other RNA-guided nucleases (e.g., modification of PAM requirements in Cas9/Cas12 CRISPR-based systems). In other embodiments, dTnpB proteins with modified TAM-interacting residues are used, in conjunction with any of the above stated embodiments, to extend the range of genomic targets.

### Example 2

### Bioinformatic Identification of Nuclease-Dead TnpB Proteins

A bioinformatics pipeline was developed to identify TnpB proteins with inactivating mutations in the RuvC domain. A multiple sequence alignment of 95,731 unique TnpB-like sequences was clustered at 50% sequence identity and then an automatic assessment of the conservation of RuvC active site residues was performed. TnpB, like Cas12 nucleases, harbors a catalytic motif consisting of three acidic residues (DED), and mutating any residue in this motif abolishes nuclease activity. However, recent analyses of TnpBs and eukaryotic TnpB-like proteins (e.g., Fanzors) indicate that one of the catalytic residues can occur at an alternate position in the RuvC domain. Indeed, it was observed that this flexibility often resulted in the spurious identification of catalytically inactivated TnpB-like proteins, since structural predictions and manual inspections suggested an intact catalytic triad. Thus, the initial analysis was restricted to TnpB-like proteins with two or more mutations in the RuvC DED motif.

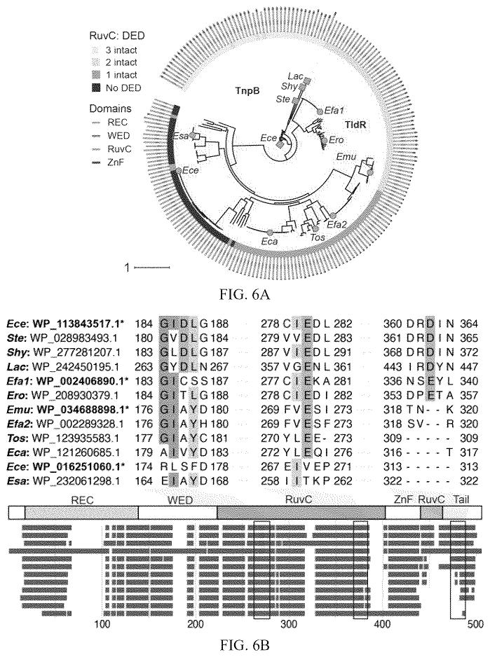

This search, supplemented with additional homologs identified in more focused analyses described below, identified over 500 TnpB-like proteins with conserved mutations that are predicted to inactivate the RuvC nuclease domain (FIG. 1B, sequences provided below). The polyphyletic distribution of these inactivated nucleases suggest that they emerged on multiple occasions independently (FIG. 1B), and based on their predicted role in transcriptional repression (see below); hereinafter referred to as TnpB-like nuclease-dead repressors (TldRs). Interestingly, TldRs exhibit a range of deteriorated active sites, with one, two or all three acidic residues mutated, and many homologs also feature truncated C-terminal domains that ablate RuvC and zinc-finger (ZnF) domains (FIGS. 1C and 6). AlphaFold predictions provided further structural support for the sequential deterioration of the RuvC active site, without any apparent degradation in the remainder of the overall TnpB/TldR fold or the RNA binding interface (FIG. 1C), suggesting that RNA-guided DNA targeting functions could be preserved for these inactivated nucleases.

### Example 3

tldRs Associate with Novel Genes and are Mobilized by Temperate Phages

Canonical tnpB genes in bacteria, alongside their ωRNA guides, are encoded within IS200/IS605- or IS607-family transposons that can be straightforwardly identified using both comparative genomics and by defining the transposon left end (LE) and right end (RE); in addition, a hallmark feature is their frequent association with tnpA transposase genes (FIGS. 2A, left). Remarkably, the genomic context surrounding tldR genes consistently lacked tnpA and identifiable LE/RE sequences, and instead, strong genetic associations were observed with non-transposon genes that were clade specific (FIGS. 1B and 2A). One TldR group is consistently associated with five to six genes encoding components of ABC transporter systems, the last of which is oppl, and is mainly present in Enterococci genomes. A second TldR group is tightly associated with fliC, a gene encoding the flagellin subunit of flagellar assemblies that propel bacteria in aqueous environments, and is found in diverse Enterobacteriaceae. A third TldR group from Clostridial genomes is similarly associated with flagellin genes, in addition to a carbon storage regulator (csrA) that is involved in flagellar subunit regulation. In all three cases, loci encoding TldRs and their associated genes were observed in varied genetic contexts, suggesting that they have maintained their associations over long time scales and/or that they have been mobilized in tandem. Strong genetic associations are also often indicative of functional coupling, indicating that TldR proteins may be involved in flagellar and ABC transporter expression and/or assembly pathways.

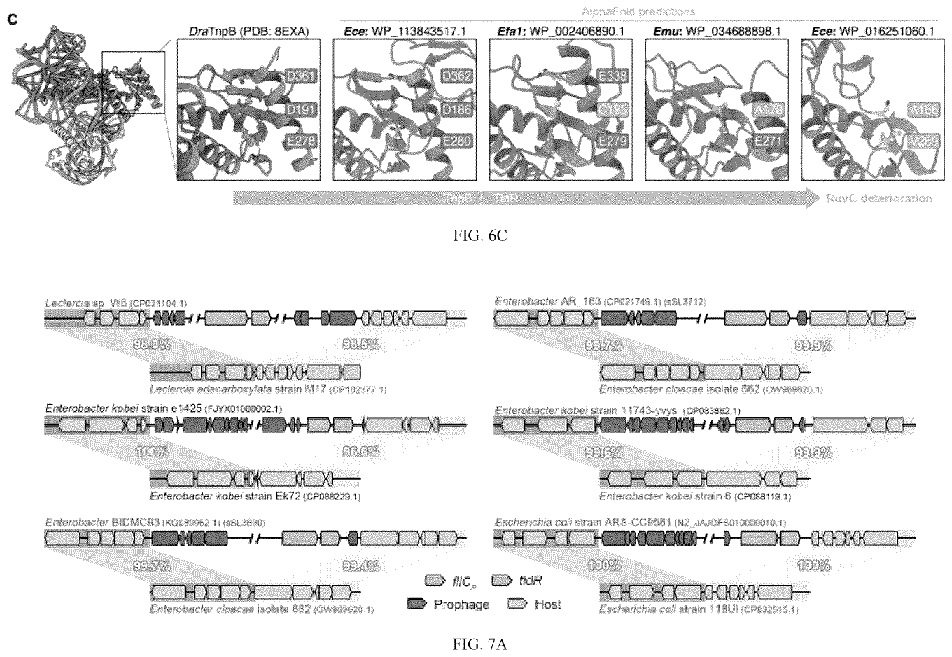

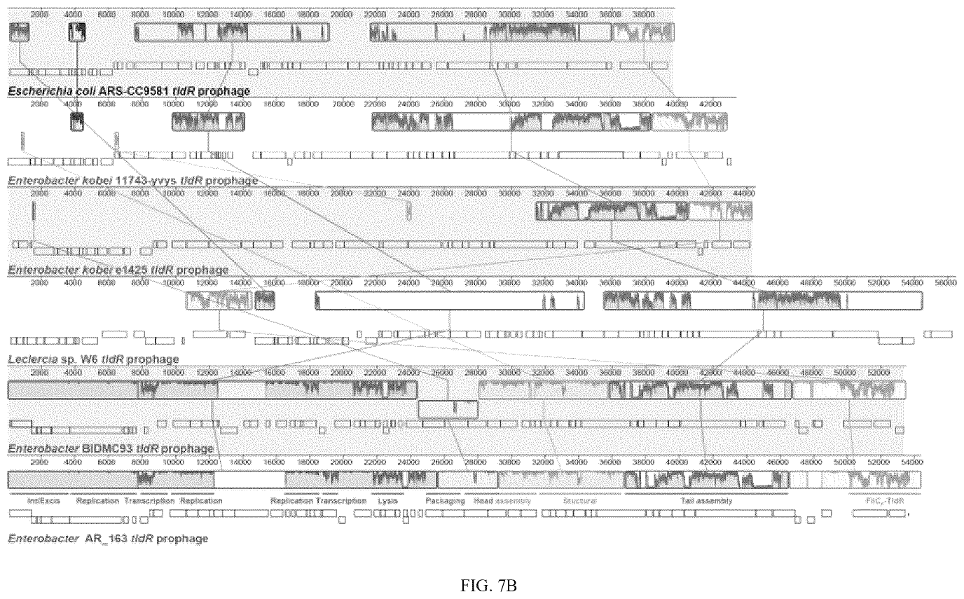

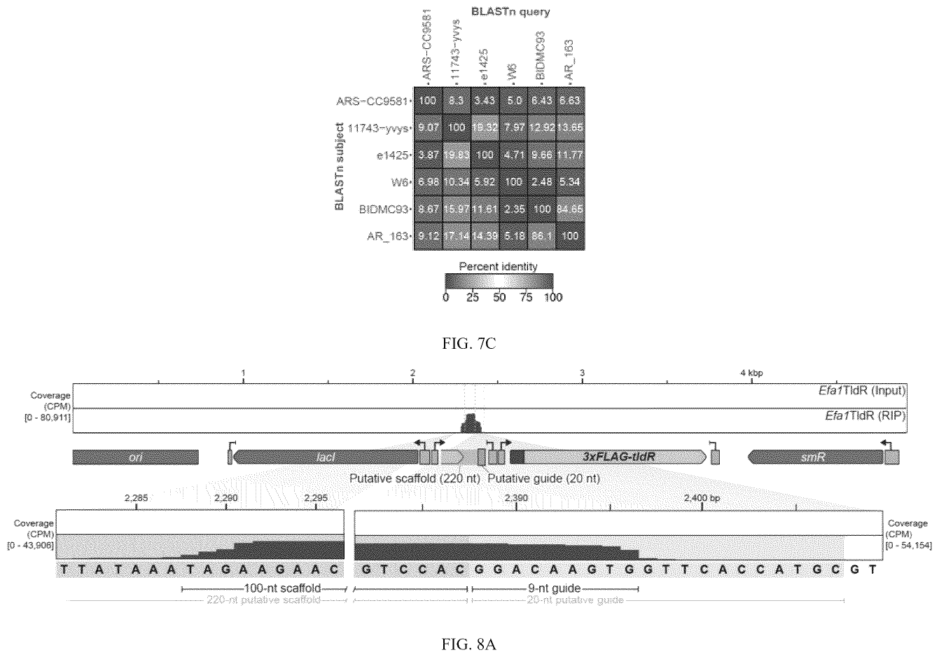

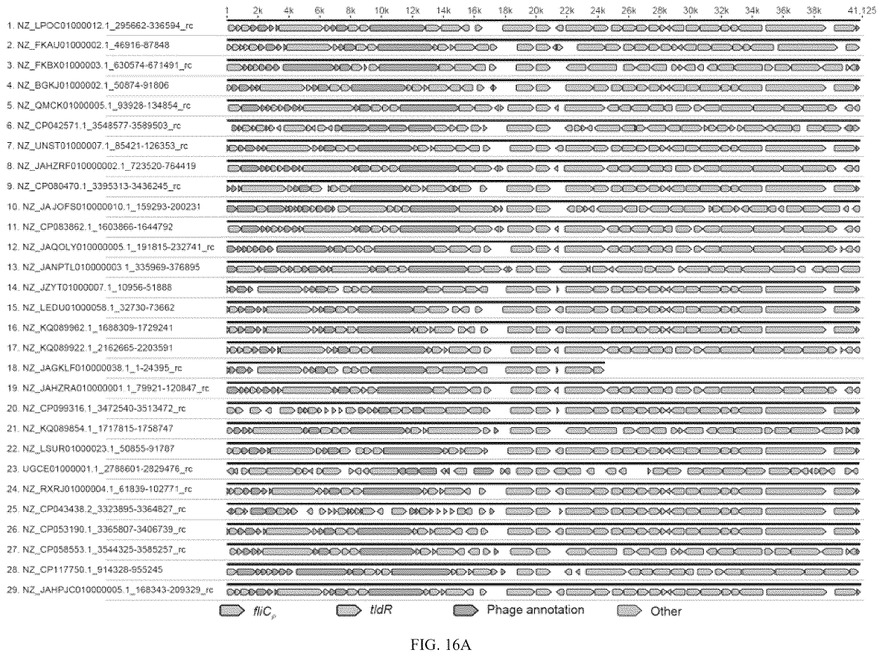

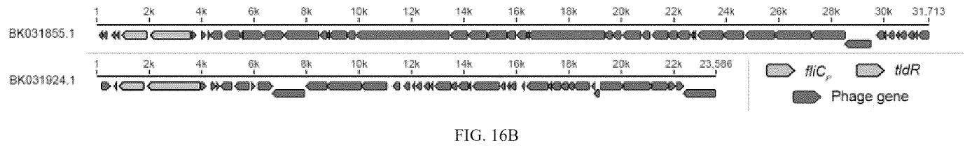

A closer inspection of genomic loci encoding fliC-tldR revealed the striking presence of numerous upstream genes with bacteriophage (phage) annotations, suggesting a potential presence of an integrated prophage (FIGS. 2A and 16A). When BLAST was used to search the NCBI non-redundant and whole genome shotgun databases, genomes were identified that were highly similar to those encoding fliC-tldR but lacked phage genes, enabling annotation of the prophage boundaries and conserved attLlattR recombination sequences (FIGS. 2B and 7A). These analyses indicate that both tldR and its associated phage-encoded fliC (hereafter fliC_{P}) are components of temperate phage genomes, suggesting a role in promoting viral infection or lysogenization. Consistent with this, the genetic association between tldR and fliC_{P} emerged coincident with the acquisition of nuclease-inactivating mutations in the RuvC domain (FIG. 2C).

To further establish the robustness of these conclusions, additional prophage elements were analyzed and it was found that fliC_{P}-tldR loci are encoded within temperate phages that, in some cases, share less than ten percent genomic sequence conservation (FIGS. 7B-7C). Additional BLAST searches revealed two metagenome-assembled phage genomes in the taxa Caudovirales that encode fliC_{P}-tldR (FIG. 16B). Collectively, these data demonstrate that at least one TnpB domestication event involved the loss of nuclease activity, the loss of flanking transposon end sequences, and the gain of an accessory gene possibly linked to a novel function in phage biology. No similar bacteriophage associations were detected for oppF- or csrA-associated TldRs.

### Example 4

Identification of TldR-Associated Guide RNAs that Target Conserved Promoters

Transposon-encoded TnpB proteins function together with gRNAs (also referred to as reRNAs) that are transcribed from within or near the 3′-end of the tnpB coding sequence, to perform RNA-guided DNA cleavage. Like CRISPR RNAs, gRNAs harbor both an invariant ‘scaffold’ sequence that is a binding site for TnpB, as well as the ‘guide’ sequence that specifies target sites through complementary RNA-DNA base-pairing. Importantly, the gRNA sequence extending beyond the transposon right end (RE) invariably comprises the guide for TnpB, and numerous in silico strategies can therefore be applied for gRNA identification, including comparative genomics, the ISfinder database, covariance models of the gRNA structure, and sequence alignments (FIG. 3A). Using these strategies, the LE/RE boundaries and gRNAs associated with nuclease-active TnpB homologs that are closely related to fliC_{P} and oppF-associated TldRs were identified (FIG. 3B). Similar analyses also revealed the predicted 3-5-bp transposon/target-adjacent motif (TAM) sequences recognized by these TnpB homologs during DNA binding and cleavage (FIG. 3B), akin to the role of PAM in DNA binding and cleavage by CRISPR-Cas9 and Cas12.

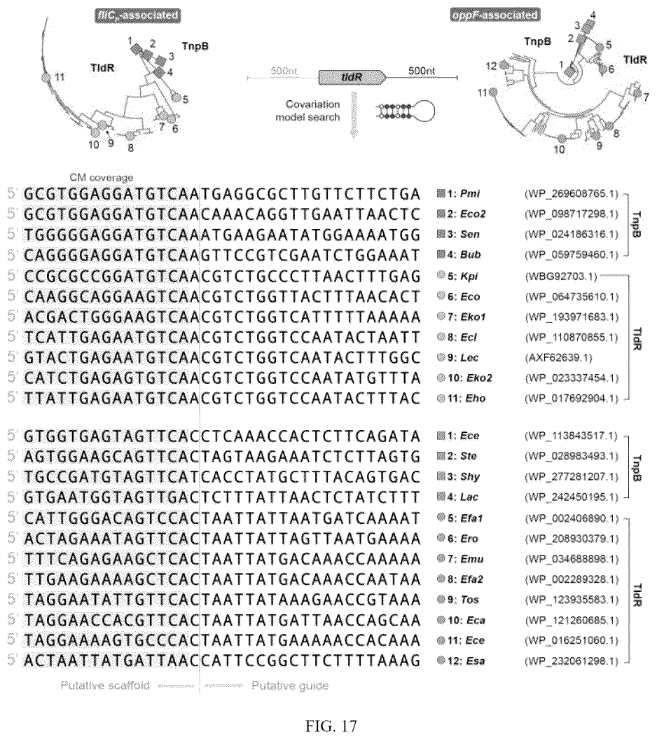

The absence of identifiable transposon ends flanking tldR rendered similar annotations of its guide RNA unfeasible, so covariance models (CM) built from gRNA sequences of related TnpBs were used. After scanning a 500-bp window flanking each tldR gene with the gRNA CM, high-confidence gRNA-like sequences were identified for both fliC_{P}- and oppF-associated tldR loci (FIG. 17). In both cases, these RNAs were encoded downstream of tldR, similar to other tpB-gRNA loci, suggesting that functional interactions with a guide RNA may have been preserved in the face of nuclease-inactivating mutations. The strong conservation at the 3′ end of the gRNA scaffold allowed further prediction of the junction between the scaffold and putative guide sequence (FIGS. 3C and 17).

Using these putative guide sequences as queries, BLAST searches were performed to identify potential genomic targets of fliC_{P}-associated TldR. The strongest match was in a genomic region that encodes other flagellar components, and strikingly, was specifically located in the intergenic region between flil) and a second (host) fliC gene distinct from the prophage-encoded fliC′p ortholog (FIG. 3D). In E. coli, fliC expression is regulated by an alternative sigma factor (σ²⁸) also known as FliA, and the putative target of the TldR-associated gRNA directly overlapped the FliA-10 promoter element, and was flanked by a conserved GTTAT motif that is highly similar to the TAM recognized by TnpB nucleases similar to TldR (FIG. 3E). Remarkably, these sequence features, similarity between the putative gRNA guide and fliC promoter, abutted by a cognate TAM, were strongly conserved across all fliC_{P}-associated loci analyzed.

When RNA sequencing datasets from organisms with fliC_{P}-tldR or oppf-tldR that are available on the NCBI short read archive (SRA) and gene expression omnibus (GEO) were analyzed, read coverage was observed over the regions identified by our CM search (FIGS. 3F-3G), additional evidence of functional gRNA expression from regions flanking tldR loci.

Collectively, these observations indicated that nuclease-inactivated tpB genes remain associated with noncoding RNA loci, and suggested a model for fliC_{P}-tldR function, wherein phage-encoded TldR-gRNA complexes could repress expression of the host FliC protein while producing their own FliC_{P} homolog. Notably, the substantial sequence differences between host and prophage-encoded FliC and FliC_{P} homologs, specifically within the hypervariable central domains, revealed the potential biological implications of this organellar transformation (see below).

### Example 5

RIP-Seq Reveals Mature gRNA Substrates and Putative OppF-TldR Targets

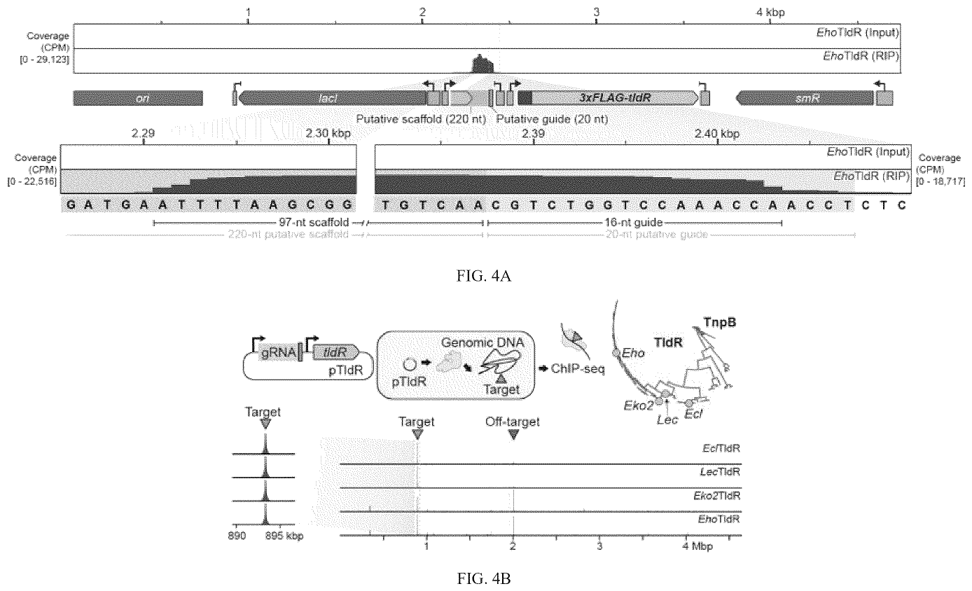

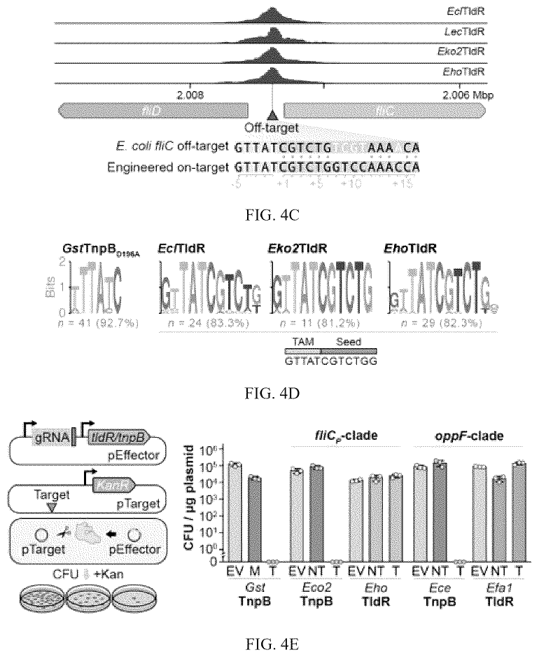

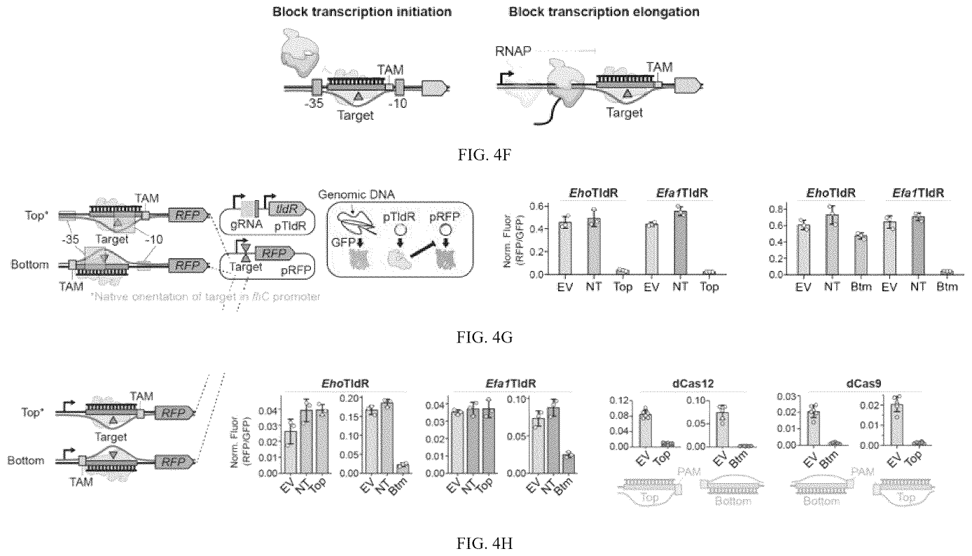

To determine if TldR proteins bind their associated guide RNAs, a representative FLAG-tagged fliC_{P}-associated TldR (EhoTldR) and oppF-associated TldR (Efa1TldR) were cloned into expression plasmids, alongside 240 bp encompassing the putative gRNA scaffold and a 20-bp guide sequence (FIG. 4A). After performing RNA immunoprecipitation sequencing (RIP-seq) and mapping reads to the E. coli genome and expression plasmid, a mature, ˜113-nt gRNA for EhoTldR that encompassed a 97-nt scaffold upstream of a 16-nt guide was identified, indicating processing from the initial transcript down to a final mature form (FIG. 4A). The absence of an intact catalytic triad in TldR proteins suggests that the mature gRNA may represent the sequence protected from cleavage by cellular ribonucleases.

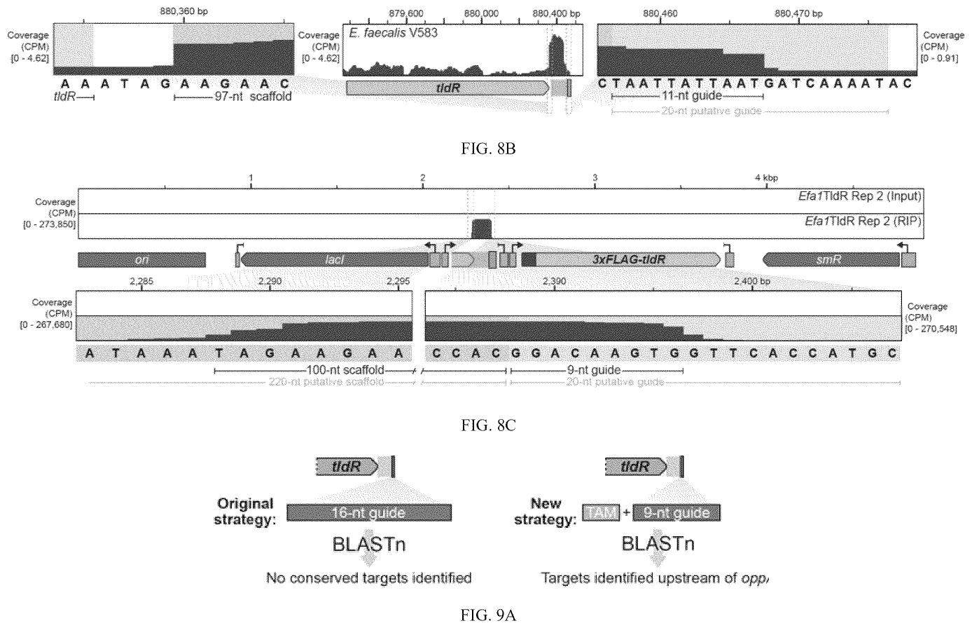

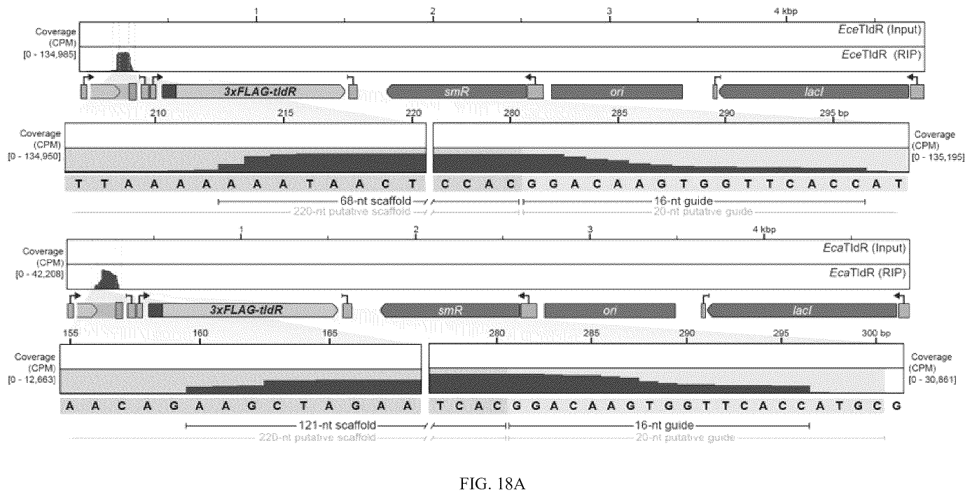

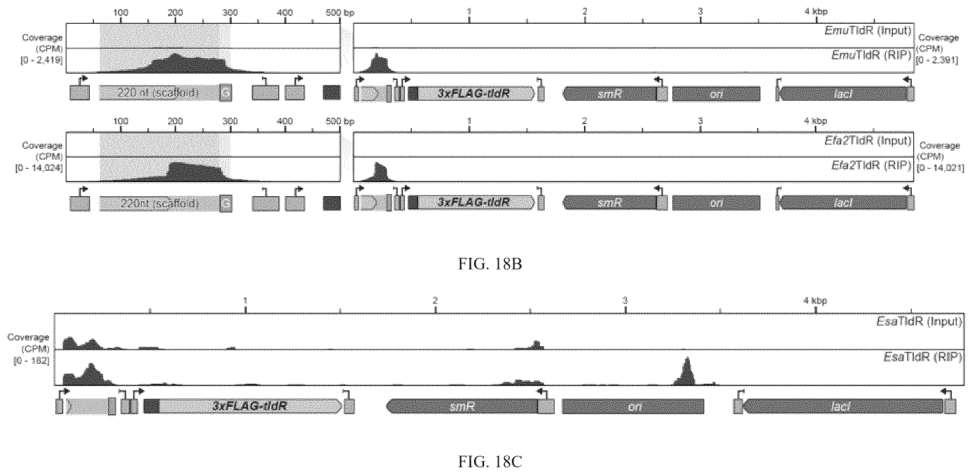

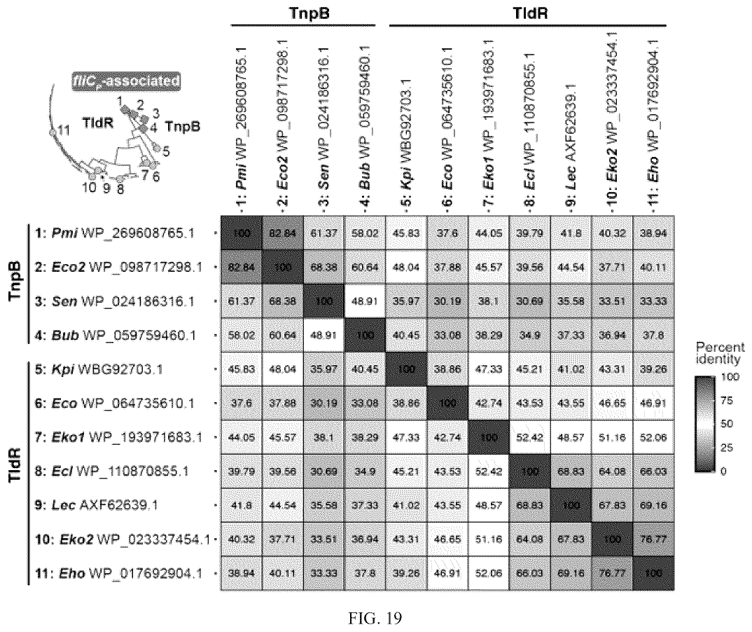

Unexpectedly, RIP-seq revealed that the oppF-associated Ffa/TldR bound an even shorter gRNA, comprising a 100-nt scaffold and ˜9-nt guide (FIG. 8A); a similarly truncated guide (11 nt) was also observed for another homolog from this clade using publicly available RNA-seq data (FIG. 8B). RIP-seq data from replicates and five additional homologs corroborated the short guide for EfaITldR while revealing more heterogeneous processing for diverse homologs, including some with guides closer in length to 16-nt, others with more diffuse peaks that rendered unambiguous determination of the gRNA boundaries challenging, and one homolog (EsaTldR) that did not appear to specifically associate with its gRNA sequence (FIG. 18).

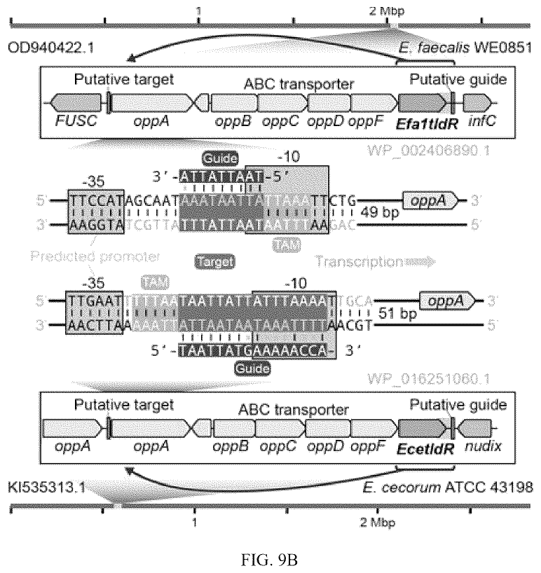

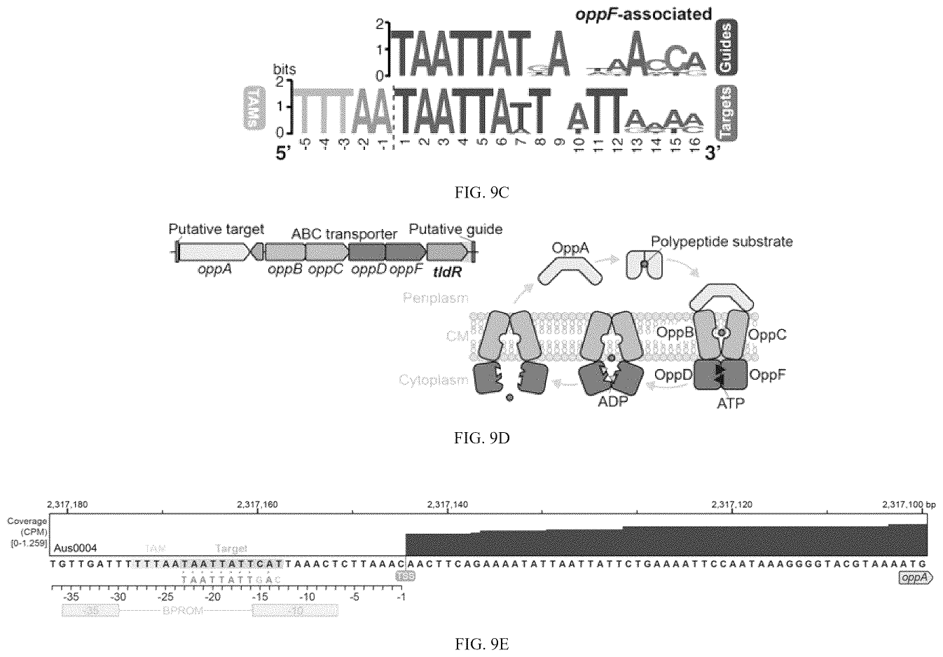

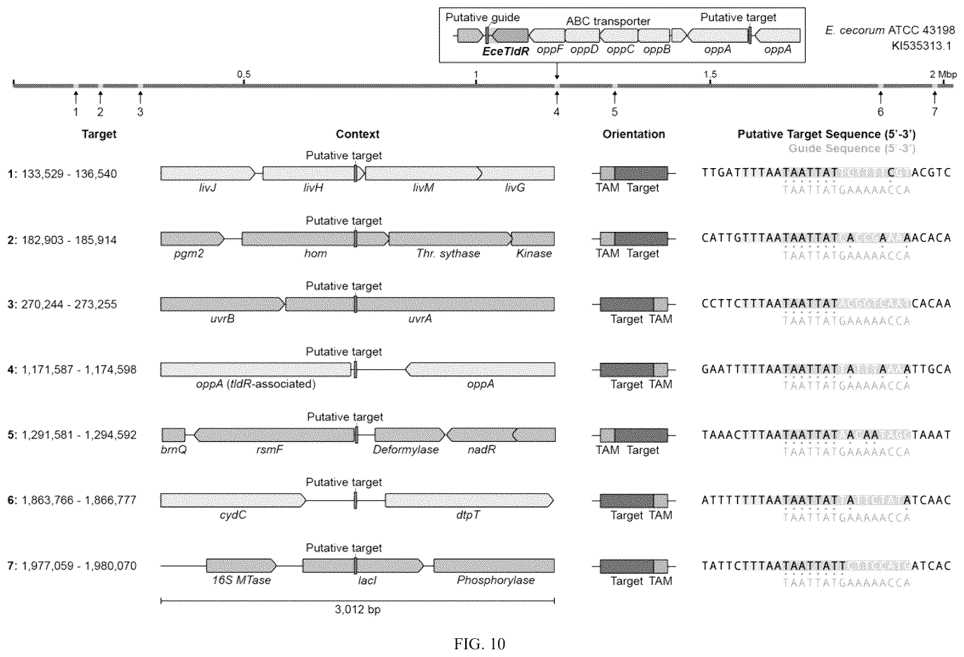

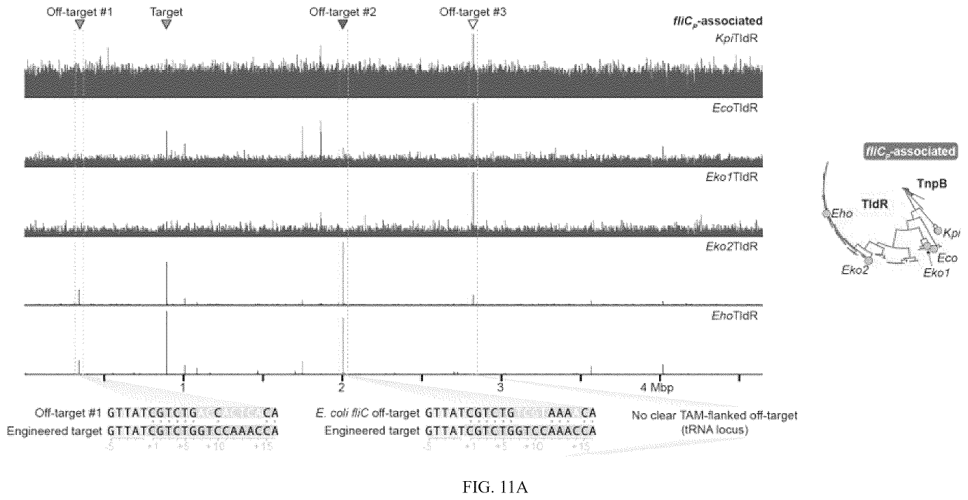

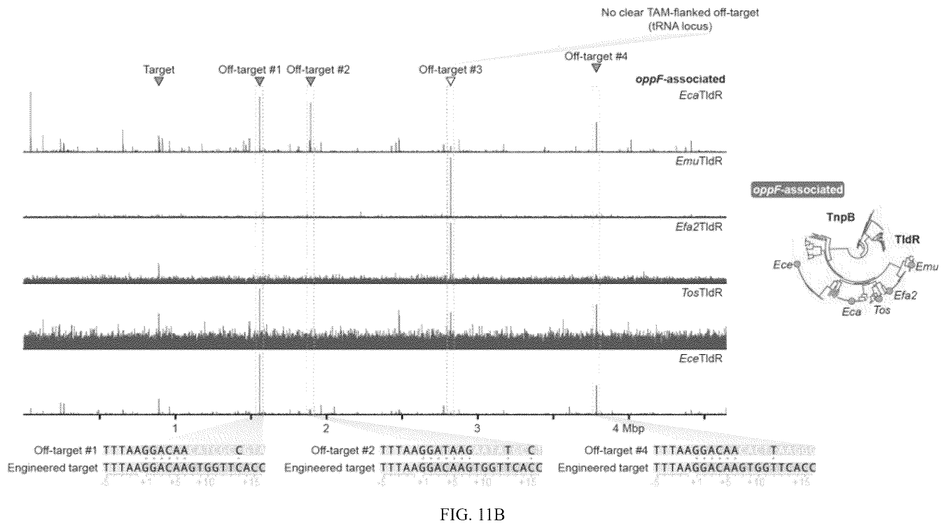

A new search for putative genomic targets was performed by screening for sites with ˜9-bp of DNA complementary to the guide flanked by a TAM similar to that recognized by related TnpB nucleases (TTTAA or TTTAT) (FIG. 9A). This analysis led to the identification of a conserved target upstream of the start codon of one of the ABC transporter genes (oppA) encoded proximally to tldR (FIGS. 9B-9C OppA is a substrate binding protein (SBP) in ABC transport systems, and tldR-associated OppA homologs are most similar to SBPs that bind short polypeptides (FIG. 9D). It was found that the putative gRNA-matching targets varied in their orientation relative to the start codon of oppA, suggesting that TldRs from this clade might be able to target either DNA strand to transcriptionally repress oppA. Bioinformatic predictions with BPROM revealed that putative TldR targets indeed overlapped with the predicted −10 and −35 promoter elements of oppA, a conclusion corroborated by analysis of RNA-seq data (FIG. 9E). Interestingly, additional putative gRNA targets were also identified in genomes encoding oppA-tldR loci, including targets upstream of other ABC transporter components, raising the possibility that TldR proteins contribute towards a more complex transcriptional regulatory network than fliC_{P}-associated TldR proteins (FIG. 10).

### Example 6

TldRs Function as RNA-Guided DNA Binding Proteins that Repress Transcription

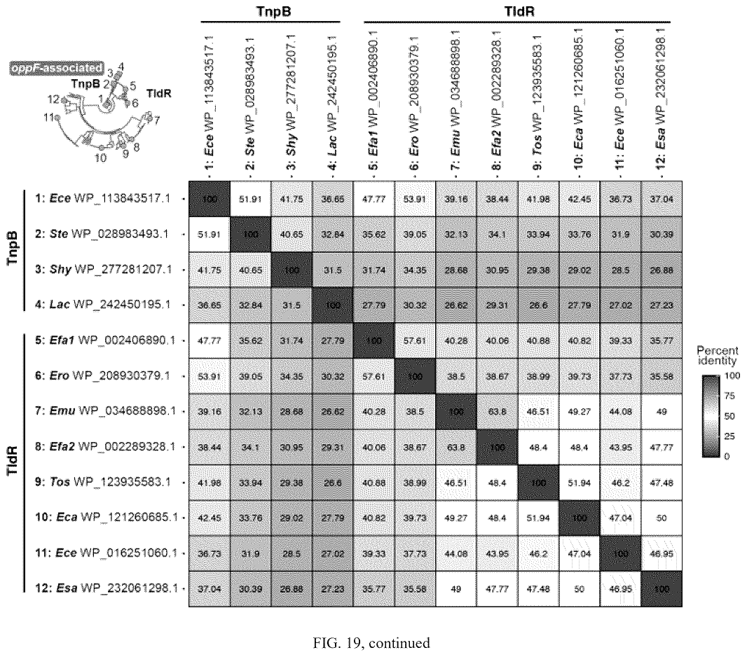

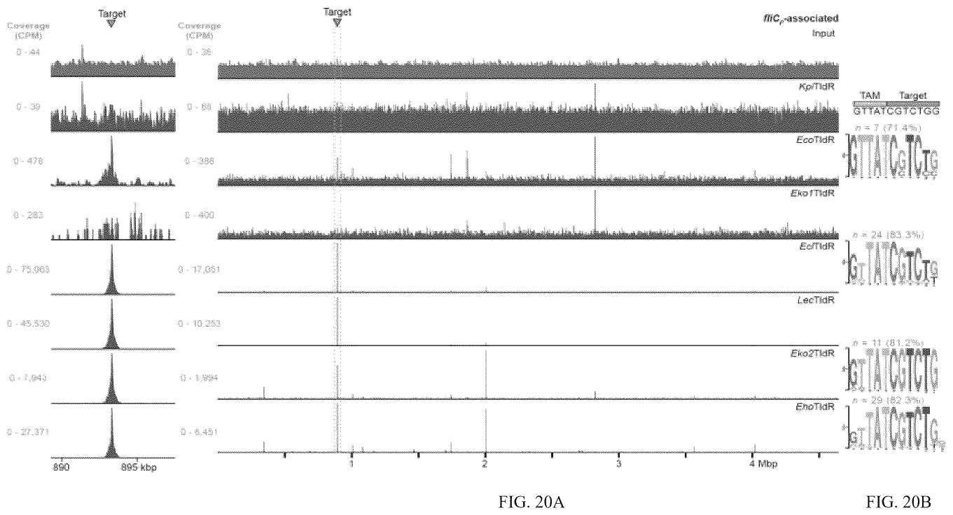

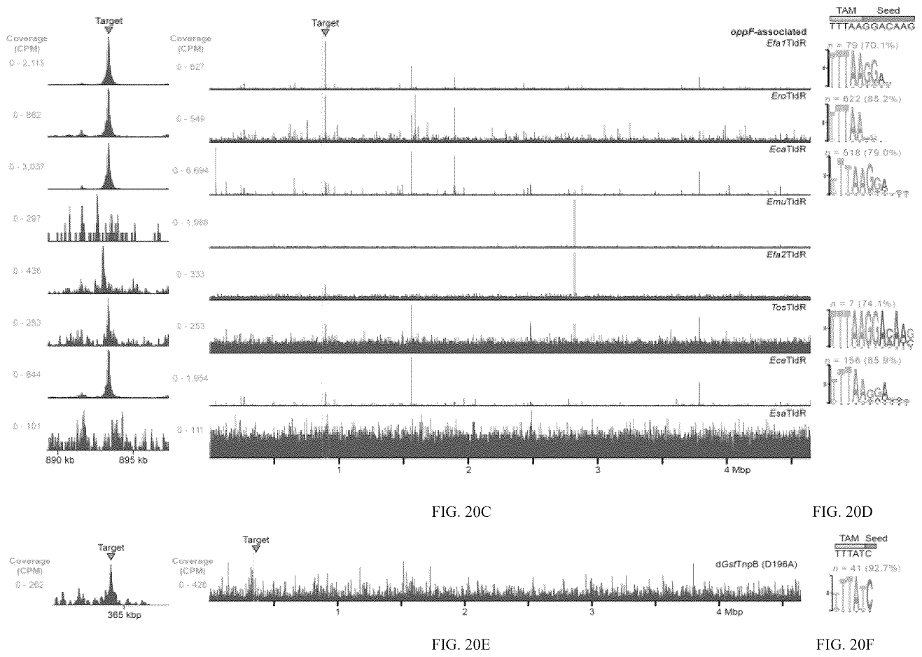

Seven fliC_{P}-associated (FIG. 2C) and eight oppF-associated (FIG. 6A) TldR homologs were selected for functional assays, which were chosen to sample the diversity within each clade (FIG. 19), each were cloned into expression vectors alongside their putative gRNAs and expressed in an E. coli K12 strain containing a genomically integrated target site. Genome-wide binding specificity was profiled using chromatin immunoprecipitation sequencing (ChIP-seq), and the resulting data revealed strongly enriched peaks corresponding to the expected target site for nearly all homologs tested (FIGS. 4B and 20). These data demonstrate that TldR proteins retain the ability to perform highly specific, RNA-guided DNA target binding in cells, despite harboring RuvC mutations and C-terminal truncations.

Prominent off-target peaks in the ChIP-seq dataset were also analyzed. One of these off-target peaks for fliC_{P}-associated TldRs corresponded to the intergenic region between E. coli fliC and flil) (FIGS. 4B-4C). The guide sequence used in these experiments is complementary to the native fli C target from Enterobacter cloacae sp. AR_154 but mutated relative to the E. coli K12 sequence at five positions (FIG. 4C), suggesting a high tolerance for TldR binding to mismatched targets (FIG. 20). Strongly enriched peaks corresponding to off-target binding for oppF-associated TldRs similarly exhibited sequence similarity across only the TAM-proximal region of the target site (FIG. 11). These data support the definition of a ˜6-nt TldR seed sequence, consistent with that seen for some Cas12a homologs.

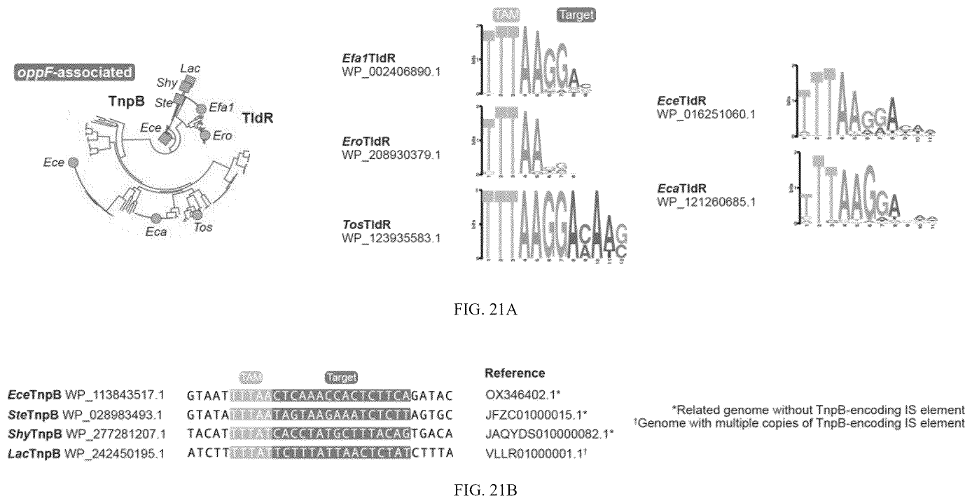

ChIP-seq also captures transient interactions due to the crosslinking step, and systematic analysis of all peaks could report on the underlying TAM specificity of select TldR homologs. Using MEME to detect enriched motifs, it was found that fliC_{P}-associated TldRs were enriched at 5′-GTTAT-3′ motifs, the same pentanucleotide TAM that flanks putative TldR-gRNA targets within fli C promoters (FIGS. 4D and 20). Similarly, oppF-associated TldR homologs bound DNA sequences enriched in 5′-TTTAA-3′ motifs, consistent with the bioinformatically predicted TAM specificities for their closely related TnpB relatives (TTTAA and TTTAT) (FIG. 21).

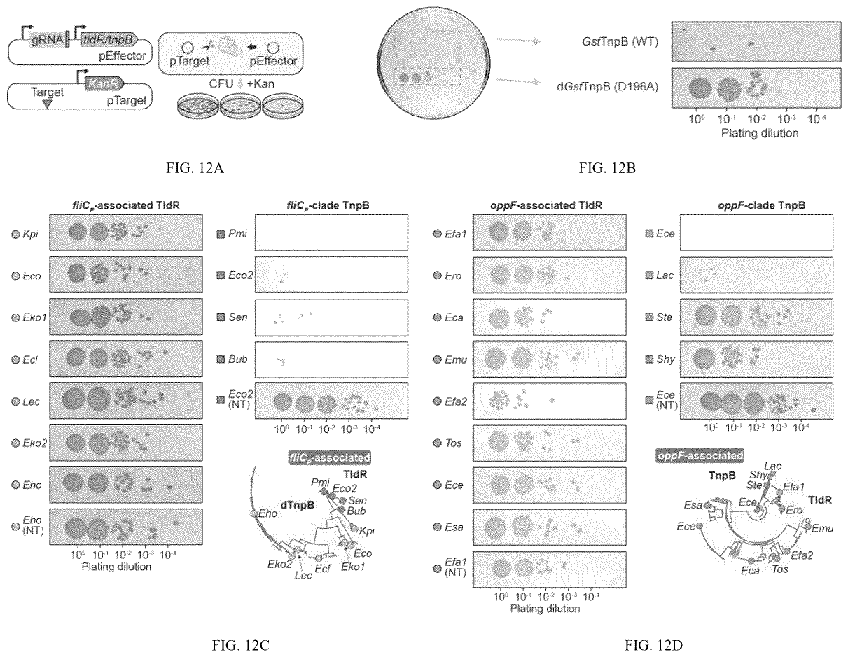

To verify that the RuvC mutations in TldR proteins abolish nuclease activity, TldR homologs or their related TnpB counterparts were tested in plasmid interference assays. Expression vectors containing TldR or TnpB and their associated gRNA (pEffector) were used to transform E. coli cells, along with a target plasmid (pTarget) bearing a kanamycin resistance cassette (kanR) and a TAM-flanked target sequence (FIG. 4E). Nuclease activity is expected to eliminate pTarget, resulting in fewer surviving colonies when cells are plated on selective media. When cells were transformed with plasmids bearing a previously studied TnpB homolog (e.g., GstTnpB3) or nuclease-active TnpB homologs similar to TldRs (e.g., EkoTnpB2 and EceTnpB), no surviving colonies were able to be isolated. This effect could be reversed using non-targeting guides or empty vector controls (FIG. 4E). In contrast, cells transformed with plasmids encoding TldR homolog exhibited similar colony counts as empty vector controls, with or without a pTarget-matching gRNA (FIG. 4E). Thirteen additional TldR homologs yielded consistent results (FIG. 12), confirming that TldR proteins function as RNA-guided DNA binding proteins that lost the ability to cleave DNA.

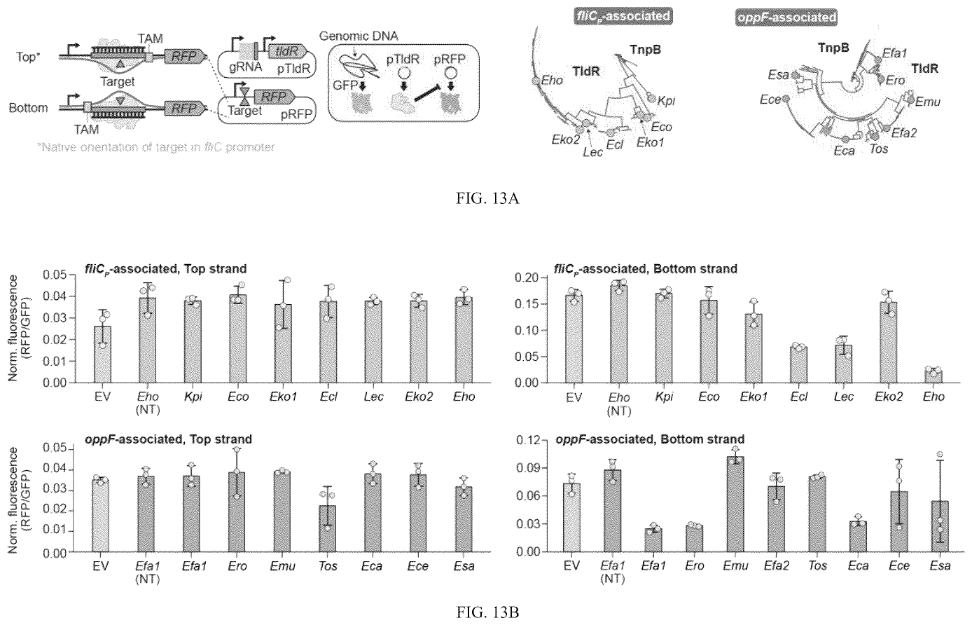

To test if DNA binding by TldR could modulate gene expression, an RFP/GFP reporter assay was developed in which target DNA binding represses rfp gene expression relative to a control gfp locus, and gRNAs were designed to either occlude transcription initiation by targeting promoter sequences, or to block transcription elongation by targeting the 5′-untranslated regions (UTR) (FIGS. 4F-4G). Representative fliC_{P}-(Eho) and oppF-associated (Efa1) TldR homologs robustly repressed RFP fluorescence when targeting the top (sense) strand, whereas only Efa1TldR repressed RFP when targeting the bottom (antisense) strand (FIG. 4G). When the 5′-UTR was targeted, select TldRs from both clades only efficiently repressed RFP when targeting the bottom strand, whereby the TAM-proximal end was oriented towards the promoter and elongating RNAP, at efficiencies that were comparable to dCas9 and dCas12 (FIGS. 4H and 13).

TldRs lack any detectable cellular nuclease activity, and instead function as RNA-guided DNA binding proteins with the potential to potently repress gene expression.

### Example 7

Prophage-Encoded tldR Genes Selectively Repress Host fliC Expression In Vivo

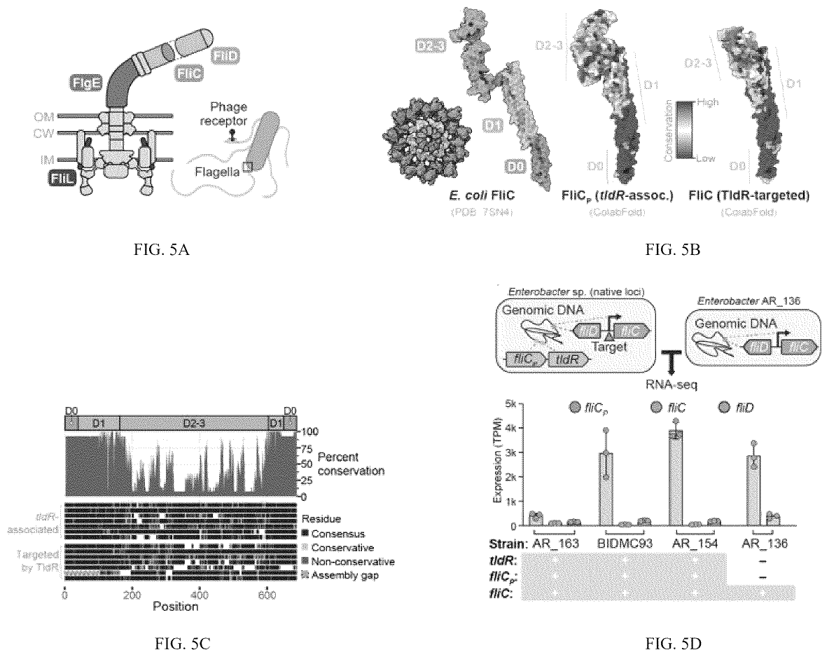

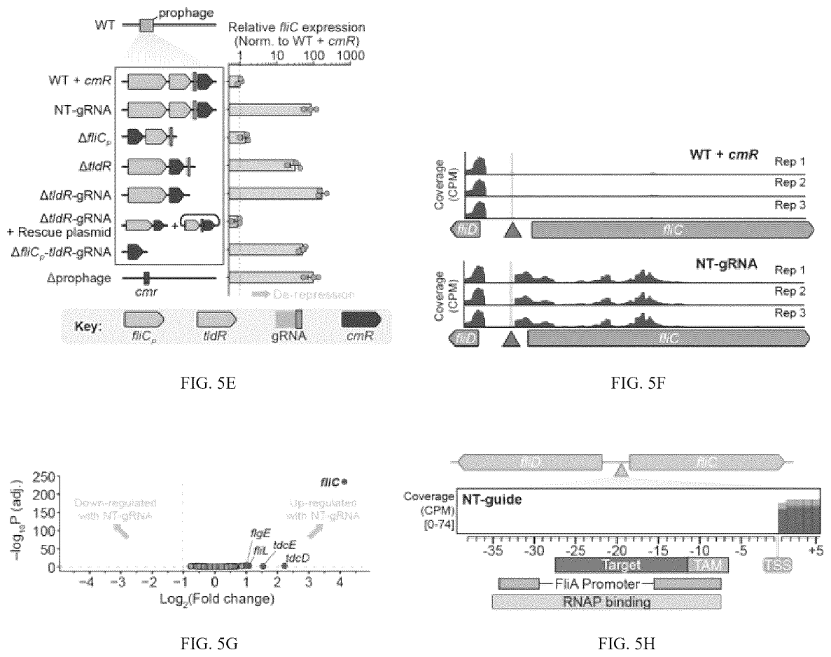

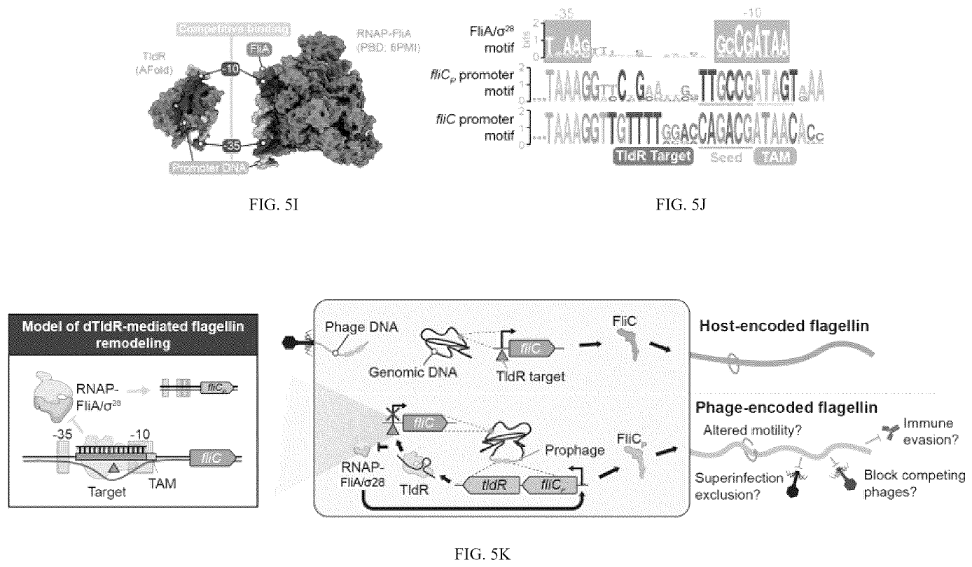

FliC, or flagellin, is the major extracellular subunit that polymerizes in tens of thousands of copies to form mature flagellar filaments, enabling bacterial locomotion (FIG. 5A). Previous structural studies defined four domains of FliC proteins, with D0 and D1 forming the majority of inter-promoter contacts during FliC polymerization, and D2 and D3 forming the central region that is predominantly exposed to the external environment (FIG. 5B). Remarkably, when comparing host FliC and prophage FliC_{P} sequences, it was found that D2-3 were highly variable whereas DO-1 were highly conserved (FIGS. 5B-5C), suggesting that prophage flagellin would likely retain the ability to form flagella together with host components, while nevertheless diversifying the chemical composition of exposed filament surfaces. Flagellin D2-3 variation has long been recognized as a potential mechanism to evade mammalian host immune systems, since FliC is a primary antigen (e.g., antigen H) decorating pathogenic bacteria. Moreover, some bacteriophages, eponymously referred to as flagellotropic phages, specifically recognize FliC within the flagellum as a primary receptor during adsorption, likely through interactions with D2-3.

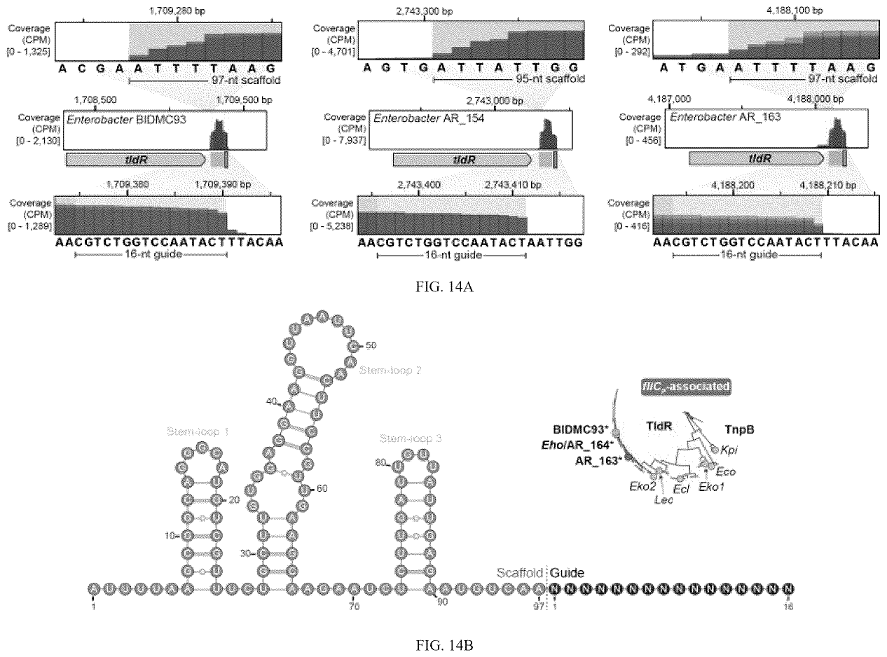

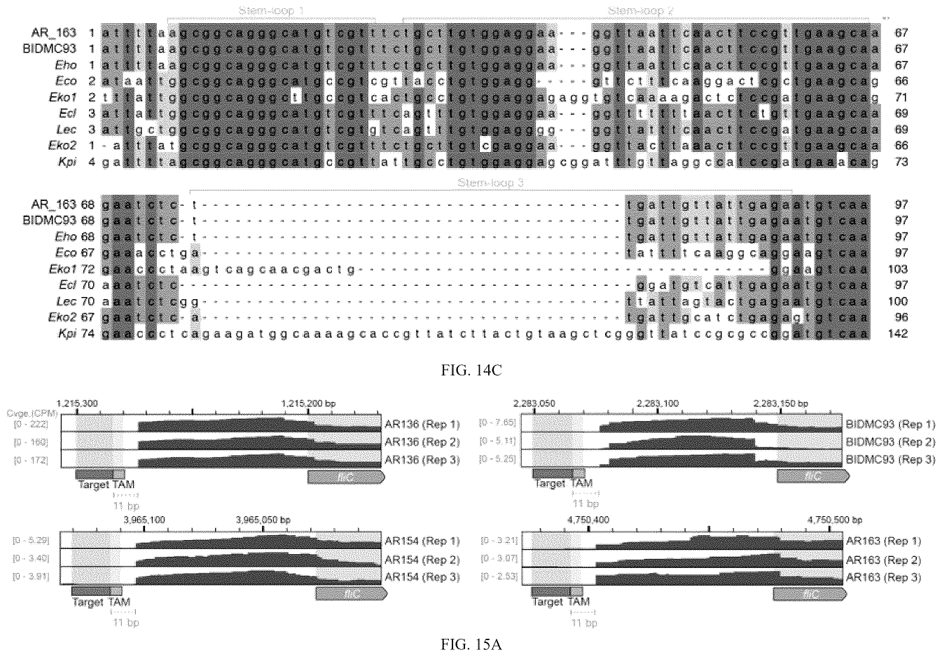

Three Enterobacter strains that each harbored a prophage-encoded fliC_{P}-tldR locus were obtained and cultured alongside a closely related control strain that lacked it and total RNA-seq was performed. Each strain with tldR exhibited robust gRNA expression, with 5′ and 3′ boundaries that were in excellent agreement with the heterologous RIP-seq data (FIG. 14). Remarkably, when flagellin gene expression was analyzed relative to the flagellar hook (flil)), it was found that host fli C was nearly undetectable in all three strains that encoded tldR whereas fliC_{P} was strongly expressed (FIG. 5D). In contrast, fliC was highly expressed in the control strain that lacked TldR and the prophage (FIG. 5D).

Precise genetic perturbations to the fliC_{P}-tldR locus were generated in Enterobacter cloacae strain BIDMC93 and the corresponding effects on host fliC expression were measured by RT-qPCR. Deletion of tldR, tldR-gRNA, the entire fliC_{P}-tldR-gRNA locus, or the entire prophage, all led to a ˜100-fold increase in host fliC expression, and the same increase was observed after substituting the guide portion of the gRNA with a non-targeting (NT) control sequence (FIG. 5E). In contrast, deletion of fliC_{P} alone had no effect, and the fliC expression increase could be reversed by complementing the tldR-gRNA deletion with a plasmid-encoded tldR-gRNA cassette (FIG. 5E). When RNA-seq was performed on isogenic strains that differed only in the guide sequence, across three biological replicates, evidence of host fliC de-repression with the NT-guide was obtained (FIG. 5F). Differential gene expression analyses further revealed that fliC was the most strongly up-regulated (e.g., de-repressed) gene transcriptome-wide (FIG. 5G), with the only other significant changes arising in genes whose expression has been linked to flagellar gene transcription.

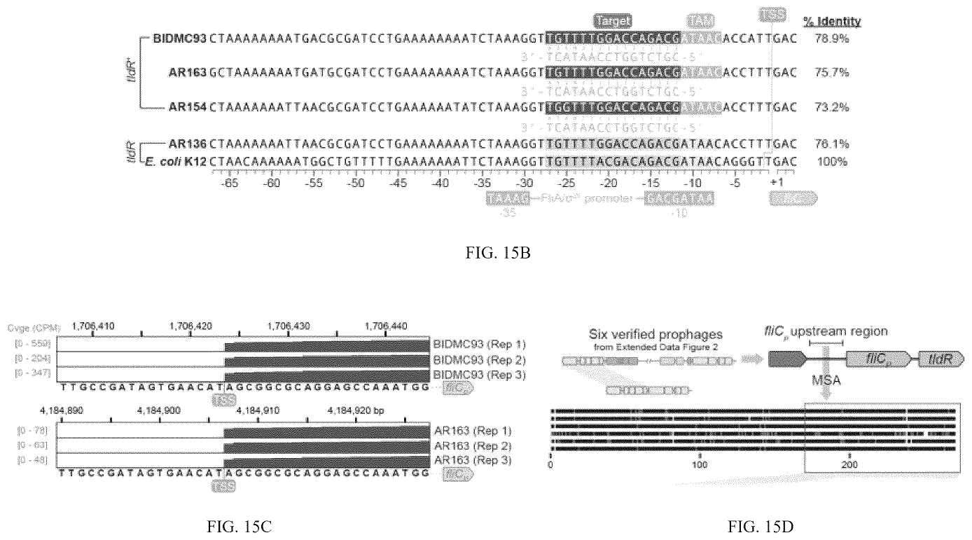

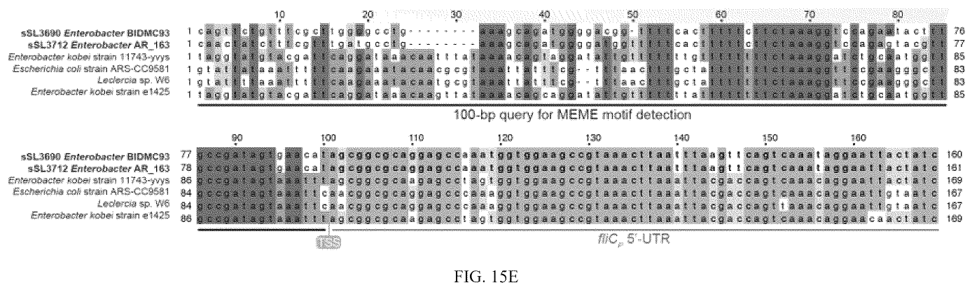

Closer inspection of the RNA-seq data lent further support that TldR represses gene expression through competitive binding to promoter elements, since the fliC transcription start site (TSS) agreed with the −35 and −10 promoter annotations informed from FliA/o data in E. coli K12 (FIGS. 5H and 15). This interpretation was also corroborated by comparisons of predicted TldR-gRNA-DNA structures with an experimentally determined RNAP-FliA-DNA holoenzyme structure, which demonstrate that TldR target binding would sterically block FliA access to DNA (FIG. 5I). To determine how prophage-encoded fliC_{P} genes would escape TldR-mediated repression, MEME was applied to detect conserved motifs in the region upstream of the experimentally-determined fliC_{P} TSS, and then Tomtom was used to compare these motifs to a database of known transcription factor binding sites. These analyses revealed that prophages likely recruit the very same host FliA/o transcriptional program to produce FliC_{P}, but with highly conserved mutations in both the TAM and seed sequence that preclude TldR-gRNA recognition (FIG. 5J). fliC_{P}-tldR locus is elegantly adapted to remodel composition of the flagellar apparatus upon establishment of a lysogen, by selectively repressing host flagellin through RNA-guided DNA targeting while hijacking cellular machinery to express its own homolog substitute (FIG. 5K).

### Example 8

csrA-Associated TldRs

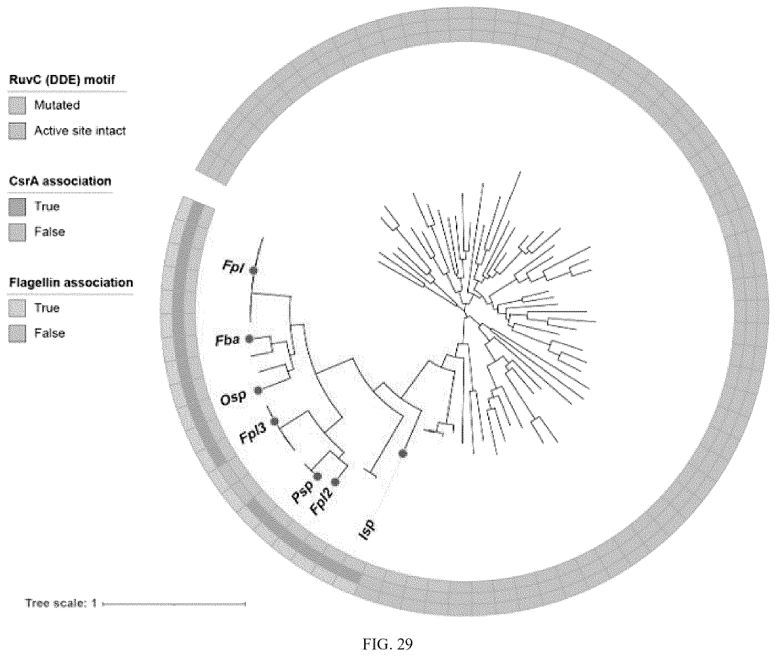

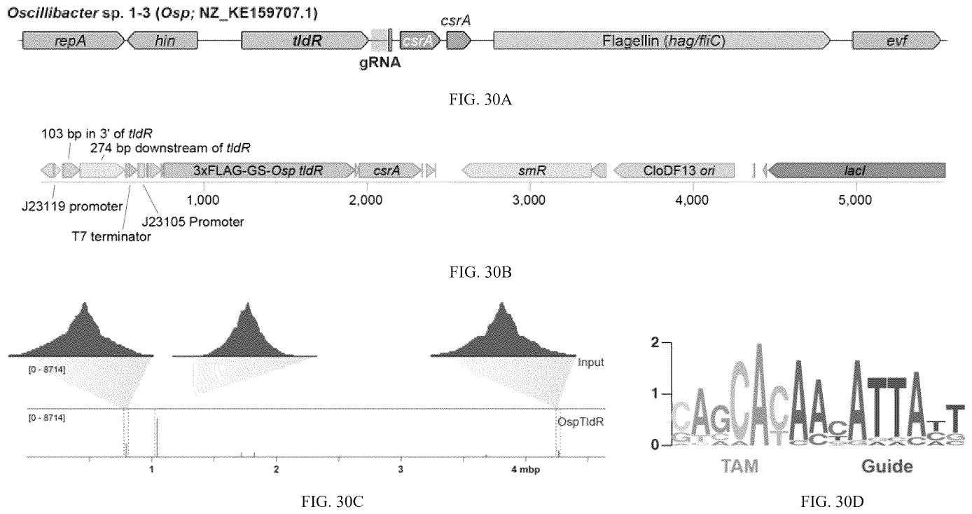

To assess the requirements for RNA-guided DNA binding of csrA-associated TldRs, seven candidates (SEQ ID NOs: 497, 500, 473, 55, 487, 496, and 39) were chosen that spanned the phylogenetic diversity of these proteins (FIG. 29; Table 5). In the native loci encoding these TldR homologs, a putative intergenic region flanking the 3′-end of tldR was speculated to encode a gRNA sequence (FIG. 30A). To determine whether or not a non-coding gRNA is present downstream of tldR, these downstream intergenic sequences (and roughly 100 bp of DNA from the 3′-end of the TldR coding sequence) were cloned into expression vectors that also encode FLAG-tagged TldR and associated csrA genes (FIG. 30B; Tables 2 and 6). These plasmids were then used to transform E. coli, and ChIP-seq was performed using an identical protocol to the methods described above for rpok-associated dCas12f proteins. When sequencing reads were mapped to the E. coli genome, coverage peaks consistent with TldR-DNA interactions that were enriched in immunoprecipitated samples, but not in input control samples were observed (FIG. 30C). Sequence motifs extracted from these peaks of ChIP-seq read coverage revealed the putative TAM sequences recognized by several TldR representatives, in addition to the 5′-end of the gRNA guide sequence utilized by csrA-associated TldRs (FIG. 30D).

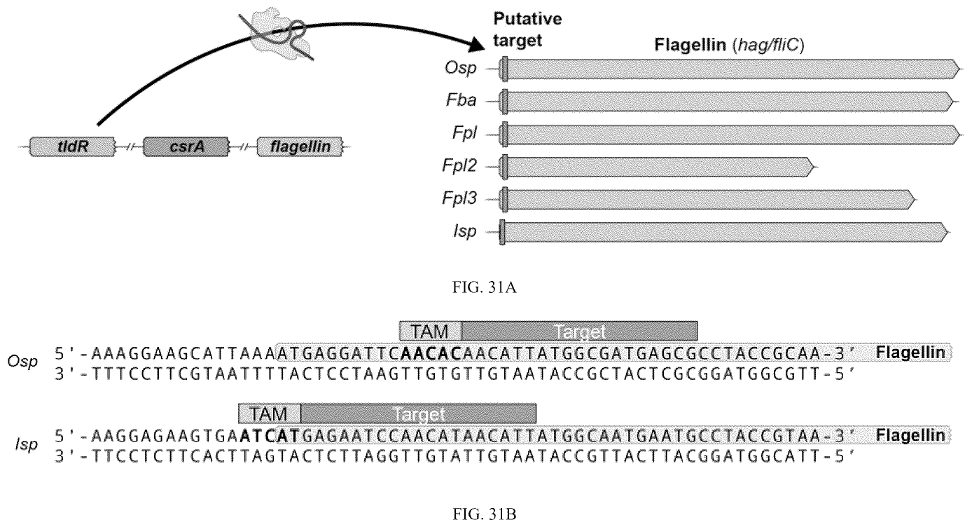

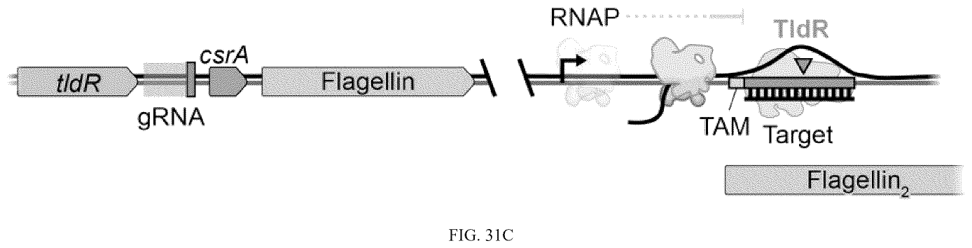

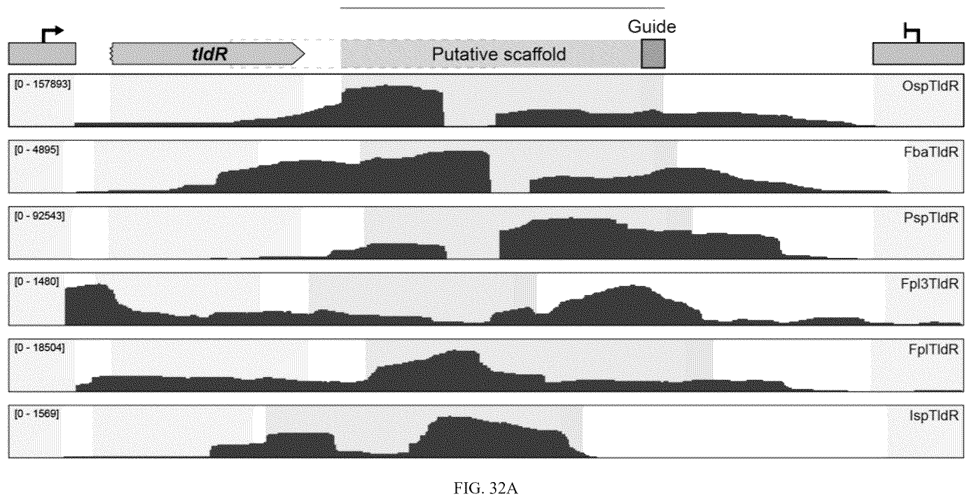

csrA-associated TldR gRNA sequence, structure and target When BLASTn was used to search genomes encoding csrA-TldRs for possible targets comprising partial the gRNA sequences identified via ChIP-seq, a conserved putative target was identified at the 5′ end of a flagellin gene (e.g., flagellin-2) that is distinct from the flagellin encoded in the csrA-tldR loci (FIG. 31A). The TAMs flanking this conserved target were additionally consistent with the putative TldR TAM preferences identified via ChIP-seq (FIGS. 30D and 31B). Collectively, these data suggest that csrA-associated TldRs specifically target flagellin-2 genes encoded elsewhere in the genome, to down regulate their expression via steric hindrance of actively transcribing RNA polymerase holoenzymes (FIG. 31C). This model of flagellar subunit regulation bears striking convergence to fliC_{P}-associated TldRs described previously.

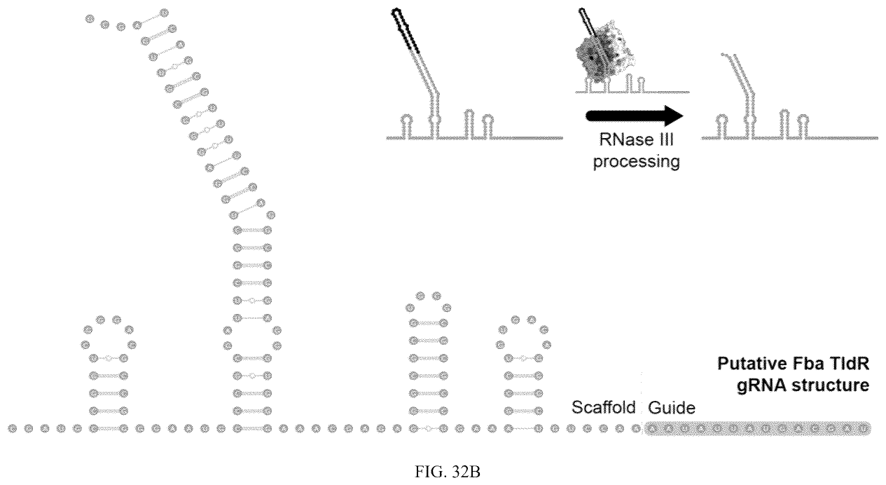

To better understand which sequences constitute the gRNAs of csrA-associated TldRs, we repeated RIP-seq using the same expression vectors used for ChIP-seq (FIG. 30B) and identical methods to those described above for rpol-associated dCas12f proteins. When sequencing reads were mapped to the tldR expression vectors, two distinct peaks were observed in the region that is expected to encode gRNA sequences for the majority of TldR homologs tested (FIG. 32A). The drop in sequencing coverage between the two RIP-seq coverage peaks suggest that part of the gRNA is processed by cellular ribonucleases (FIG. 32B), such as RNase III, which cleaves long RNA hairpins and for maturation of Cas9 gRNAs in type II CRISPR-Cas systems. Unexpectedly, RIP-seq coverage also extended beyond the 3′-end of TldR guide sequences for some homologs (FIG. 32A), suggesting that processing at the 3′-end of the gRNA is variably efficient in E. coli for this clade of TldRs.

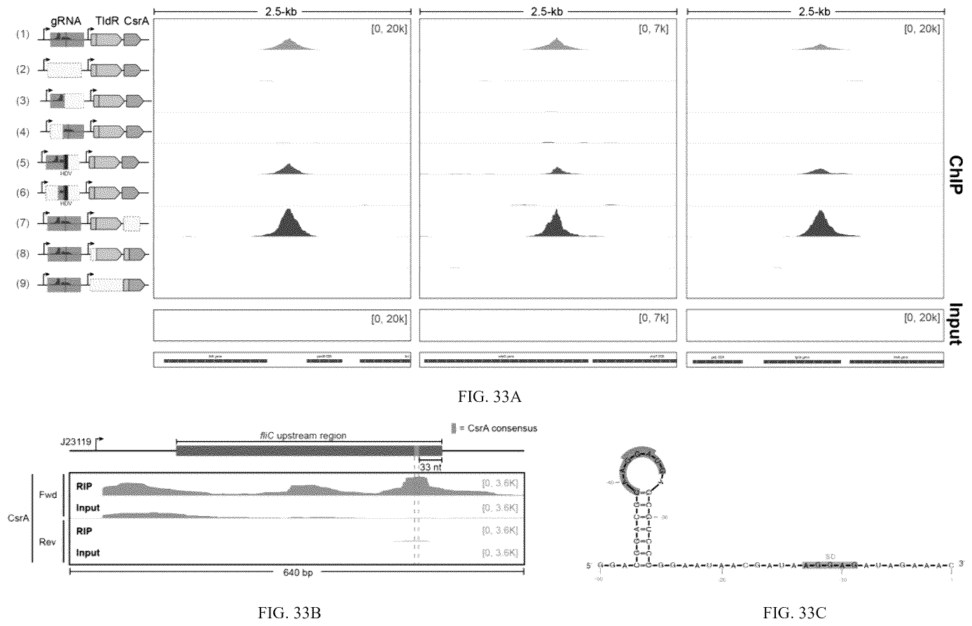

To determine whether or not this sequence downstream of the expected gRNA facilitates TldR-DNA interactions, a number of gRNA expression mutants were assayed for DNA binding using an identical ChIP-seq protocol to the experiments described above. When the region downstream of the expected gRNA was deleted, and a hepatitis delta virus ribozyme sequence was added to the 3′-end of the guide sequence to ensure RNA processing at this junction, ChIP-seq profiles remained consistent with profiles obtained from our original expression vector that included this downstream sequence (FIG. 33A). These data suggest that no sequences beyond the 3′-end of the guide sequence are required for TldR-mediated DNA binding. However, when the sequence corresponding to the first peak in RIP-seq coverage of the gRNA expression region was deleted from tldR-gRNA expression vectors, ChIP-seq reads corresponding to TldR-DNA interactions were abolished (FIG. 33B). Instead the ChIP-seq profiles of these mutants was consistent with the read profile of samples where the gRNA was deleted from the tldR expression vector altogether (FIG. 33B). These findings are consistent with the hypothesis that this upstream region is part of the gRNA scaffold, which is likely processed into a split gRNA via RNase III-mediated cleavage of a long stem loop (FIG. 32B).

### Example 9

Sigma Factor E (rpoE)-Associated, Nuclease-Dead Cas12f Systems

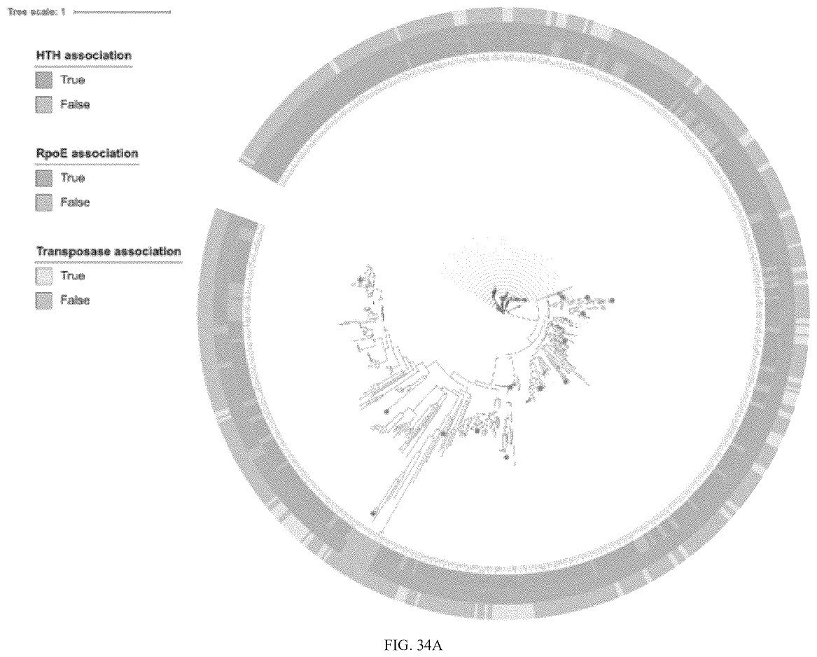

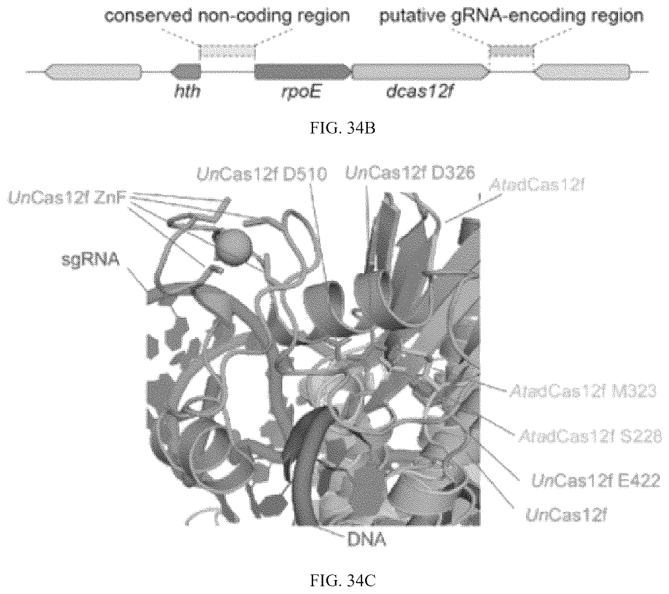

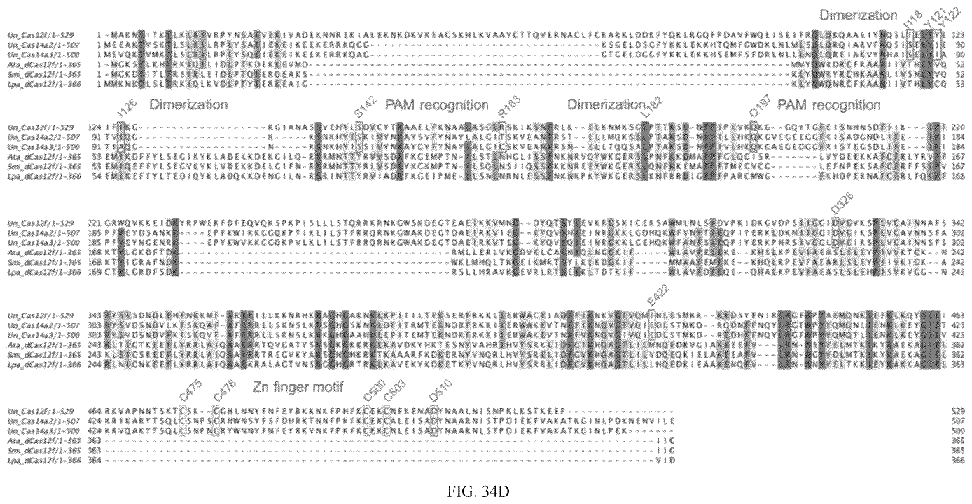

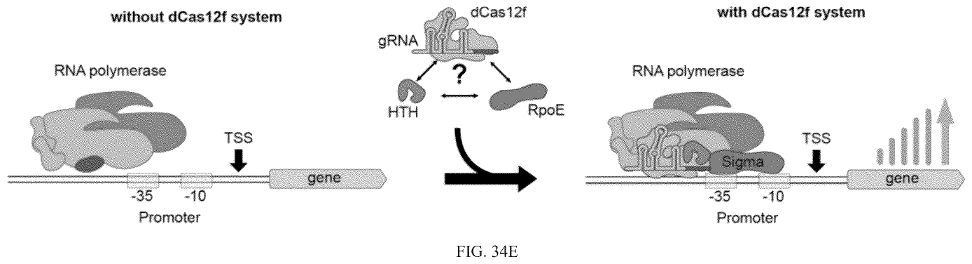

Using phylogenetic analyses, over 600 unique protein-coding genes related to the RNA-guided endonuclease Cas12f were identified, primarily in the bacterial phylum Bacteroidetes/Bacteroidota (FIG. 34A). These cas12f-like genes are encoded directly downstream of a Sigma factor E (rpok:) gene (FIG. 34B). Sigma factors are proteins that constitute an essential part of the transcription machinery by forming a complex with RNA polymerase (RNAP) and directing it to the promoter region of genes to facilitate transcription initiation. Sigma factors recognize and bind the −35 and −10 elements, upstream of the transcription start site (TSS). Sigma factor E (RpoE or extracytoplasmic function (ECF) Sigma Factor) is used by bacteria to respond and (up-) regulate gene expression under stress conditions. In addition to a gene encoding for RpoE, the cas12f-like genes also have a conserved association with a small helix-turn-helix (HTH) protein-coding gene, upstream of the rpoE gene, separated by an intergenic region approximately 75-3,000 bp in length. This sequence space is named the ‘conserved non-coding region’ and may encode for a non-coding RNA or regulatory sequence. The hth gene is encoded on the opposite strand compared to cas12f and rpoE. Notably, the annotated cas12f genes code for miniature proteins, compared to canonical (InCas12f proteins, with a typical length around 330-400 amino acids. Furthermore, structural predictions using AlphaFold2 indicate that Cas12f is catalytically dead (nuclease-dead Cas12f or dCas12f) due to mutation of more than one of the three catalytic residues (aspartate, glutamate, aspartate; DED) and/or by C-terminal truncation of the last catalytic residue glutamate (FIGS. 34C and 34D).

The close genetic association of deas12f with rpok and hth suggested the proteins may act together as a functional unit, wherein the nuclease dead Cas12f protein binds to a cognate gRNA to target a specific DNA locus, without DNA cleavage, in a programmable fashion. RpoE, in complex with dCas12f bound to gRNA, may be recruited to the same DNA target site along dCas12f. For example, at this target site, RpoE acts as a transcription initiator to upregulate transcription of the target-adjacent gene (FIG. 34E).

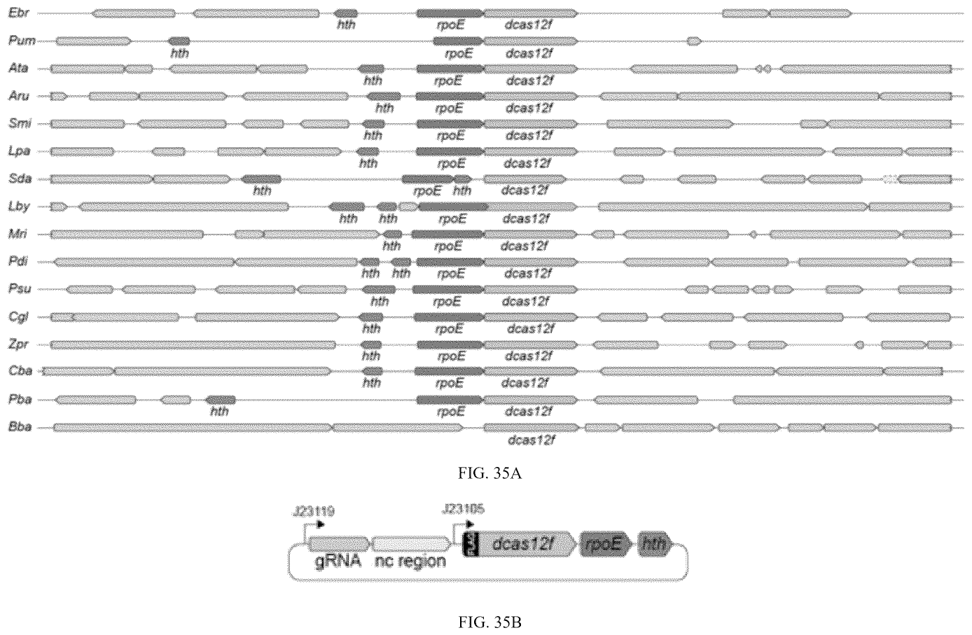

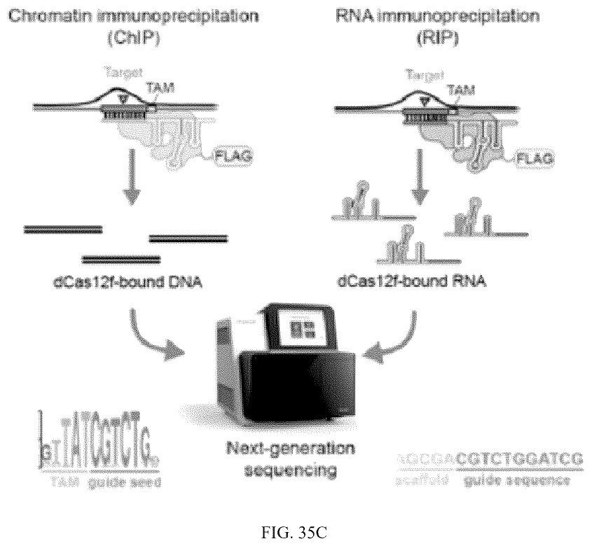

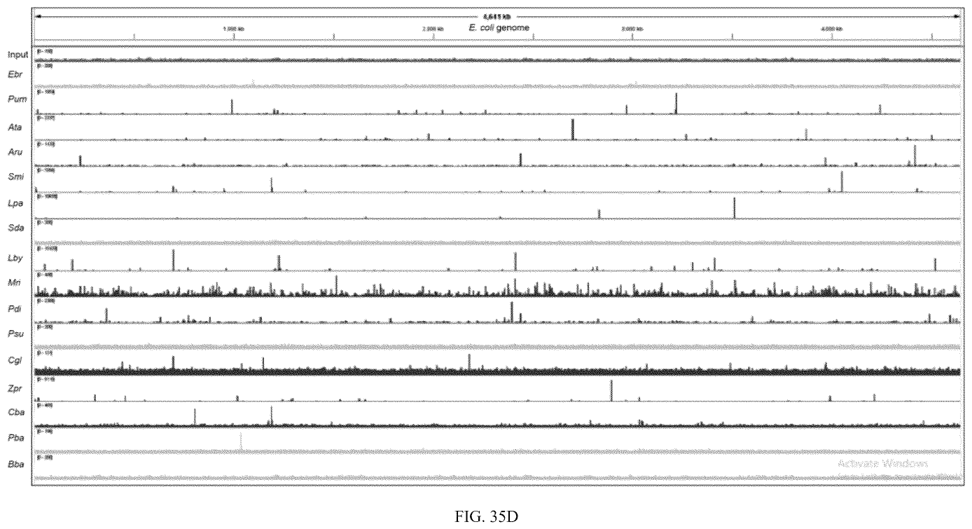

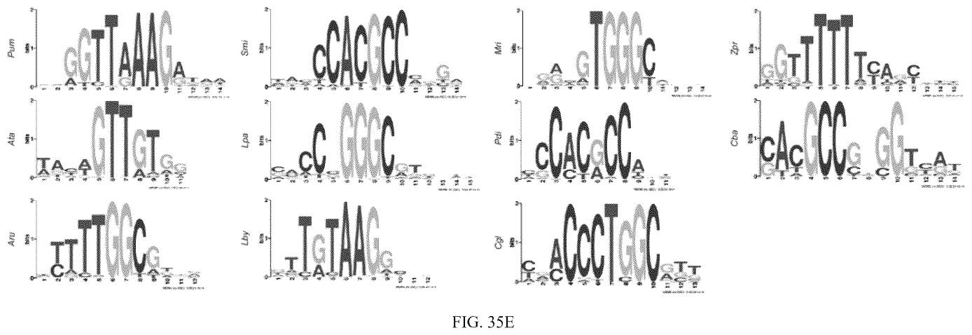

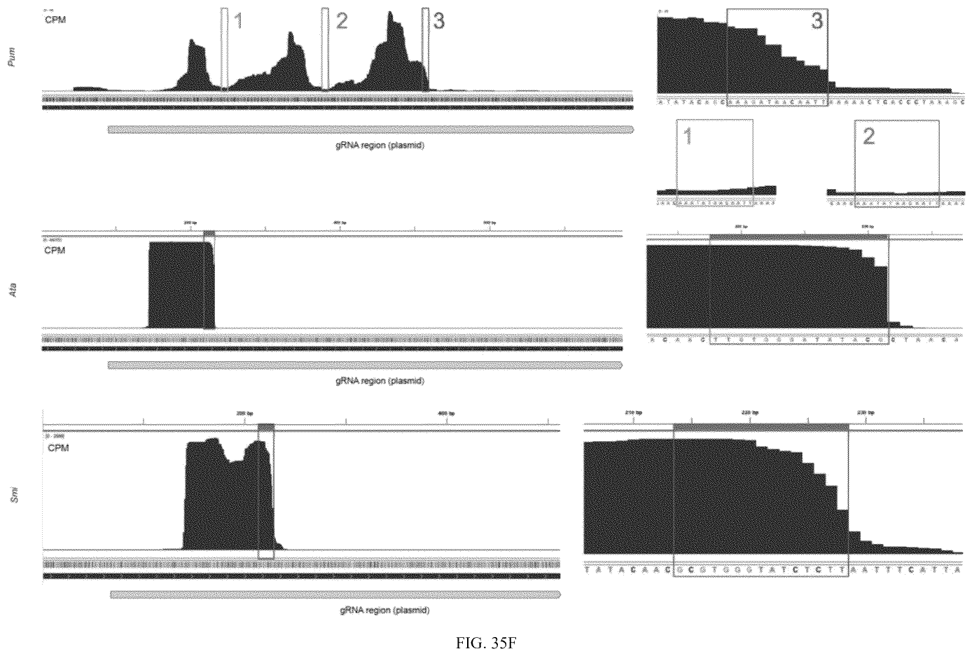

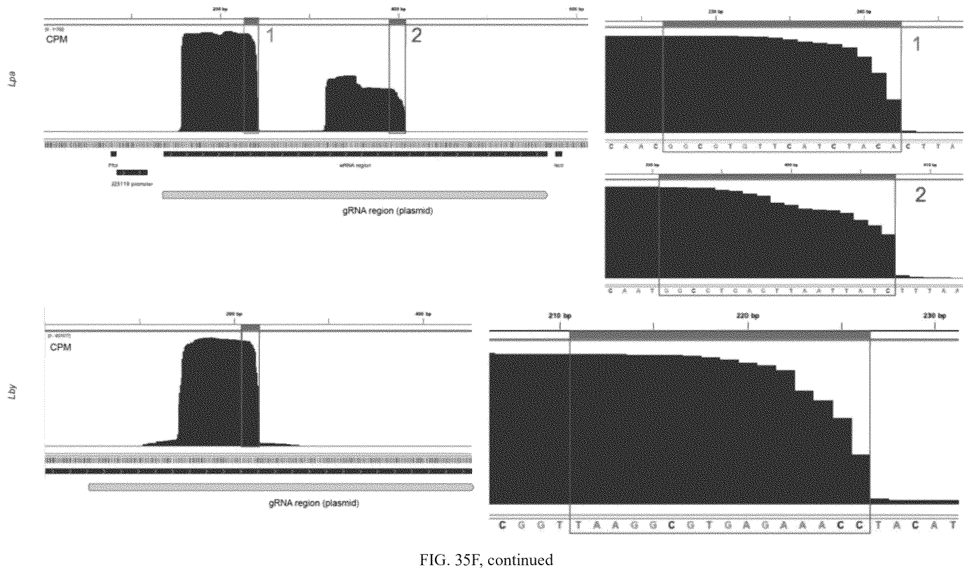

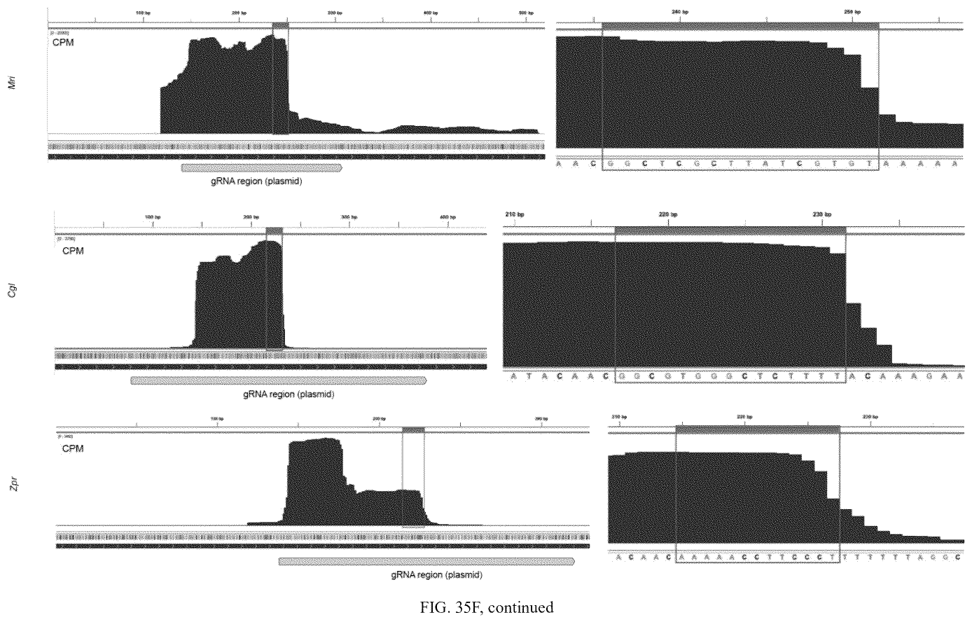

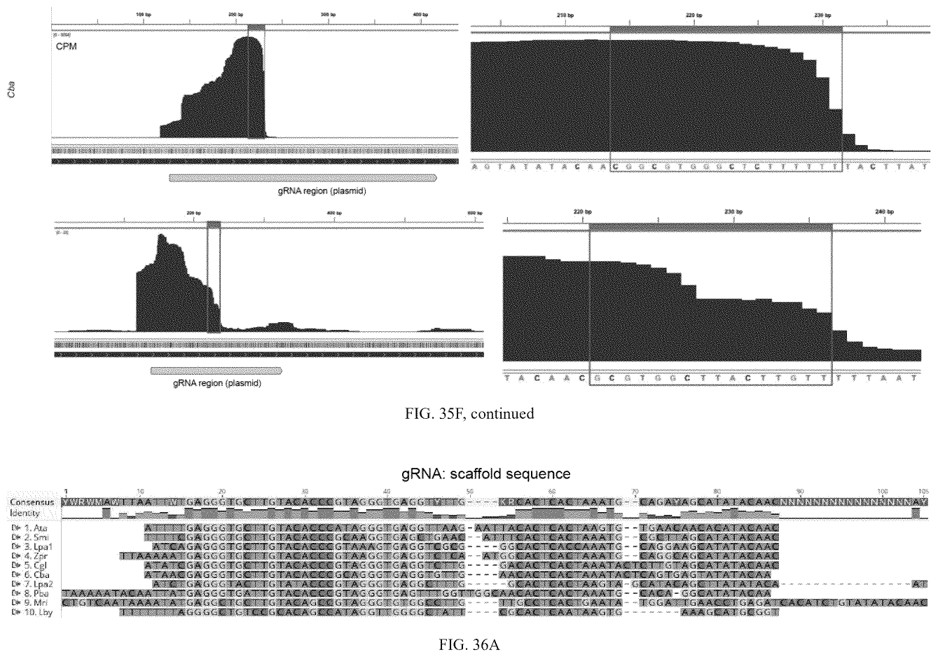

Determining nucleic acid requirements for RNA-guided DNA targeting of RpoE-associated dCas12f To assess whether a gRNA is expressed downstream of dCas12f, 16 diverse RpoE-associated dCas12f systems were selected from across the phylogenetic tree (FIG. 34A) for gene synthesis, cloning and heterologous expression in E. coli (FIG. 35A). Protein sequences for dCas12f, RpoE and HTH can be found in Table 7. For simplicity, each homolog system was provided with a three-letter code, representing the species of origin (e.g., Ata for Allomuricauda taeanensis). For systems with two hth genes, protein sequences are listed as HTH1 and HTH2. The two non-coding regions, including (a) the putative ‘gRNA region’ directly downstream of the dcas12f stop codon until the start codon of the next gene, and (b) the ‘conserved non-coding region’ in between the start codons of hth and rpok, were cloned downstream of a constitutive J23119 promoter. Further downstream, on the same plasmid, all protein-coding genes, das12f with an N-terminal 3×FLAG-tag, rpok, and hth, were cloned under the control of a separate constitutive J23105 promoter (FIG. 35B). All plasmid sequences used for E. coli experiments can be found in Table 2.

Chromatin immunoprecipitation followed by sequencing (ChIP-seq) was performed to determine DNA sites targeted by dCas12f in the E. coli genome. In parallel, RNA immunoprecipitation followed by sequencing (RIP-seq) was used to determine the mature gRNA bound to dCas12f (FIG. 35C). For both methods, the 3×FLAG tag on dCas12f was used as an epitope for immunoprecipitation.

For ChIP-seq, E. coli K-12 substrain MG1655 cells were transformed with the homolog system plasmids described above. Cells were grown for 16-24 h at 37° C. on solid or in liquid media, resuspended in 40 ml LB media and crosslinked with 1 ml of 37% formaldehyde (Thermo Fisher Scientific), at a final concentration of ˜1% formaldehyde. The crosslinking agent was quenched with 2.5 M glycine (˜0.25 M final concentration). Cell pellets were washed twice with 40 ml TBS buffer (20 mM Tris-HCl pH 7.5, 0.15 M NaCl) and cells equivalent to 40 ml of OD600 nm=0.6 were aliquoted. For each sample, 25 ul of Dynabeads Protein G (Thermo Fisher Scientific) were crosslinked in 1×PBS buffer (Gibco) supplemented with 5 mg/ml BSA (GoldBio) to 4 ul of anti-Flag M2 antibodies produced in mouse (Sigma-Aldrich) for at least 3 h at 4° C. In the meantime, crosslinked cell pellets were sonicated using a Covaris LE220 ultrasonicator with the following SonoLab settings: min. temp. 4° C.; set point 6° C.; max. temp. 8° C.; peak power: 420; duty factor: 30; cycles/burst: 200; 17.5 min sonication time. After conjugating, the antibody-magnetic beads were added to the sonication supernatant and incubated at 4° C. for 12-16 h. Then, the magnetic beads were washed and immunoprecipitated protein bound to crosslinked DNA was eluted. Reverse-crosslinking was performed at 65° C. overnight. Samples were treated with RNase A (Thermo Fisher Scientific) and proteinase K (Thermo Fisher Scientific) and purified using QIAquick spin columns (QIAGEN). ChIP-sequencing libraries were generated using the NEBNext Ultra II DNA Library Prep Kit for Illumina (NEB). Size selection (˜450 bp fragment size) was performed using AMPure XP Beads (Beckman Coulter) and samples were sequenced using the Illumina NextSeq 500 platform in paired-end mode with 75 cycles per end. Sequencing reads were mapped to the E. coli K-12 genome (GenBank NC_000913.3) using bowtie2 and normalized using deepTools bamCoverage and visualized in IGV using counts per million (CPM). MACS3 was used to call peaks, from which the 200 bp surrounding the peak summit were extracted and used as input for MEME-ChIP to determine DNA sequence motifs bound by dCas12f.

RIP-seq was performed similarly to ChIP-seq, but without cross-linking. Cells equivalent to 20 ml of OD600 nm=0.5 were aliquoted and washed using TBS buffer and lysed by sonication. RNA was extracted using TRIzol (Invitrogen) and purified using the RNA Clean and Concentrator Kit (Zymo). RNA was fragmented by heat, followed by RppH (NEB) and DNase (Thermo Fisher Scientific) treatment. 5′ ends were phosphorylated and 3′ ends were repaired. 3′ and 5′ adapters were ligated and reverse-transcription primers hybridized. RIP-sequencing libraries were prepared using the NEBNext Small RNA Library Prep Set for Illumina. Samples were sequenced as described above for ChIP-seq. Sequencing reads were mapped to the E. coli K-12 genome and expression plasmids using bwa-mem2 and normalized and visualized as described for ChIP-seq.

Visualization of ChIP-seq reads in IGV revealed distinct enrichment sites (peaks) across the E. coli genome for the majority of the samples, indicative of stable and specific dCas12f binding events (FIG. 35D). Bioinformatic analysis of the DNA sequences within the called peaks using MEME-ChIP revealed sequence motifs selectively bound by dCas12f, that are shared across genome-wide peaks (FIG. 35E). Those motifs likely comprise a combination of (a) DNA base pair(s) recognized via protein-DNA recognition by the protein dCas12f, called target-adjacent motifs (TAMs), akin to the recognition of protospacer-adjacent motifs, or PAMs, by canonical CRISPR-Cas systems; and (b) DNA sequences recognized by the complementary gRNA via RNA-DNA base-pairing, and in particular the seed portion of the guide, which is known to base-pair with the target DNA strongest in related CRISPR-Cas systems.

To distinguish between the TAM and guide portion, RIP-seq reads were visualized. To assess whether a gRNA was expressed from the ‘gRNA region’ or ‘conserved non-coding region’, RIP-seq reads were mapped back to the expression plasmid. Indeed, for most of the 16 homolog plasmids, strong enrichments were observed within the ‘gRNA region’, strongly supporting the existence of functional gRNAs that associate with the various dCas12f proteins (FIG. 35F). Furthermore, motifs identified by MEME-ChIP could be clearly located within the 3′ end of RIP-seq coverage, the region traditionally harboring guide sequences for canonical and well-studied type V CRISPR-Cas systems. By comparing the MEME-ChIP motifs and RIP-seq coverage as well as the underlying plasmid sequence of the ‘gRNA region’, the TAM and gRNA sequences of 9 out of 16 dCas12f homologs were determined (Table 8). The TAM and gRNA of a 10th system was identified in absence of a clear MEME-ChIP motif, by manual inspection (Pba homolog). Strikingly, no RIP-seq coverage was observed for the ‘conserved non-coding region’ suggesting that RpoE-associated dCas12f systems operate using a single gRNA. However, the Pum homolog had three distinct RIP-seq coverages within the ‘gRNA region’ potentially suggesting the presence of three functional gRNA that can be bound to dCas12f. Similarly, the Lpa homolog showed two even more well-defined RIP-seq enrichments within the ‘gRNA region’, indicative of a gRNA cluster composed of two gRNAs encoded downstream of the das12f gene (FIG. 35F).

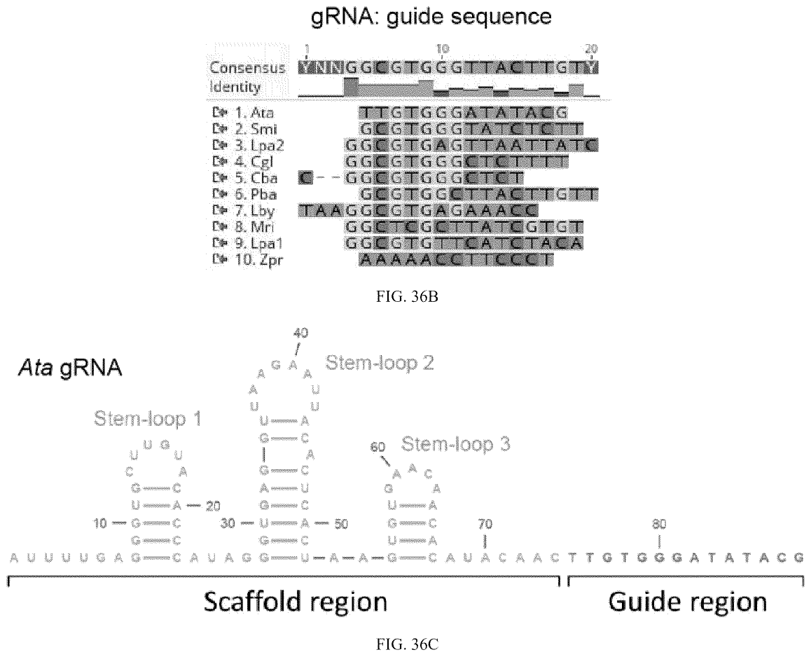

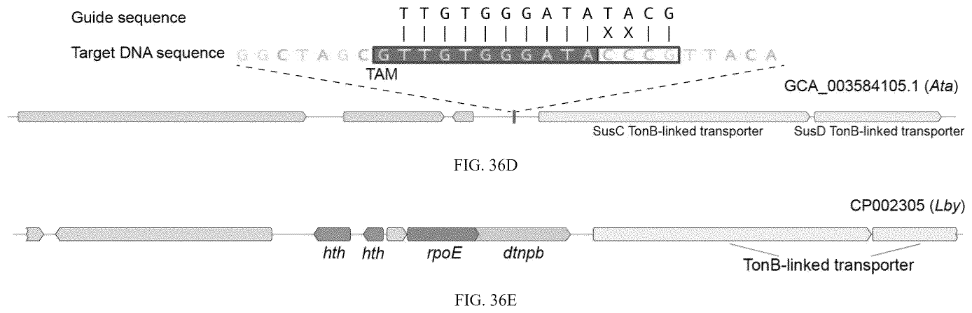

dCas12f gRNA sequence, structure, and target Notably, gRNAs of most systems are similar in length, ranging between around 75-120 nt. A sequence alignment of gRNAs of similar length revealed general sequence conservation of the scaffold region (FIG. 36A). This also applies to the guide portion which shares striking sequence conservation (FIG. 36B). By searching the reference genomes of organisms natively encoding the chosen dCas12f homolog systems, a clear DNA target site for the gRNA was identified for the Ata homolog. The structure for this 88-nt gRNA, including its 14-nt guide portion, was predicted (FIG. 36C). AtadCas12f targets around 250 bp upstream of a susC gene (FIG. 36D). susC encodes for a TonB-dependent receptor protein SusC that is involved in transport across the outer membrane (OM) in bacteria. Furthermore, genes linked to TonB can be found in proximity to a number of the chosen dCas12f loci (FIG. 36E) and are commonly also regulated by their own set of sigma factors, including RpoE. In summary, by targeting upstream of susC, dCas12f may be involved in regulating its gene expression.

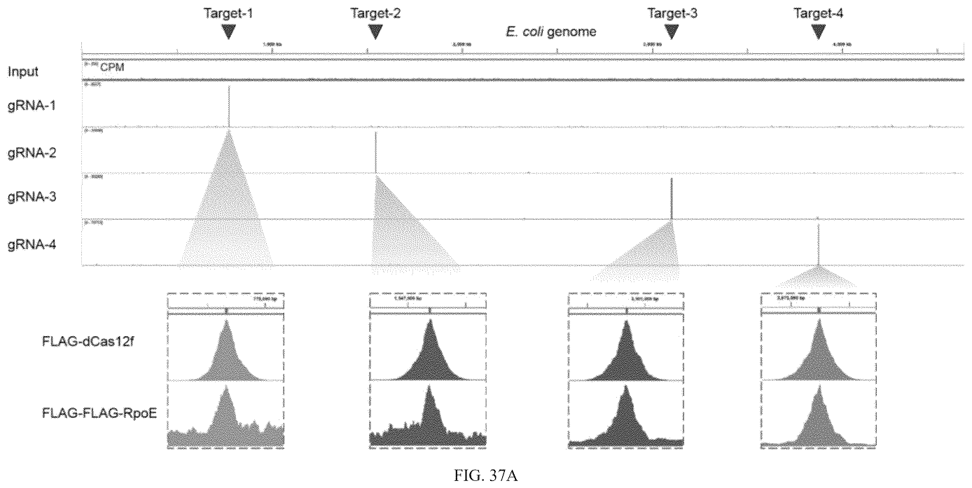

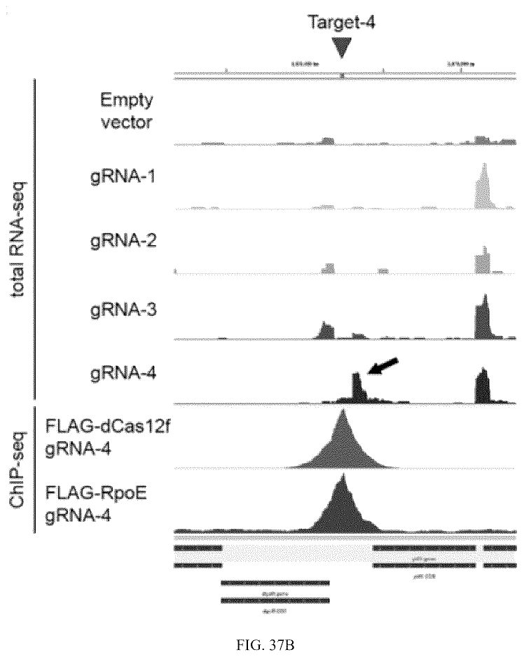

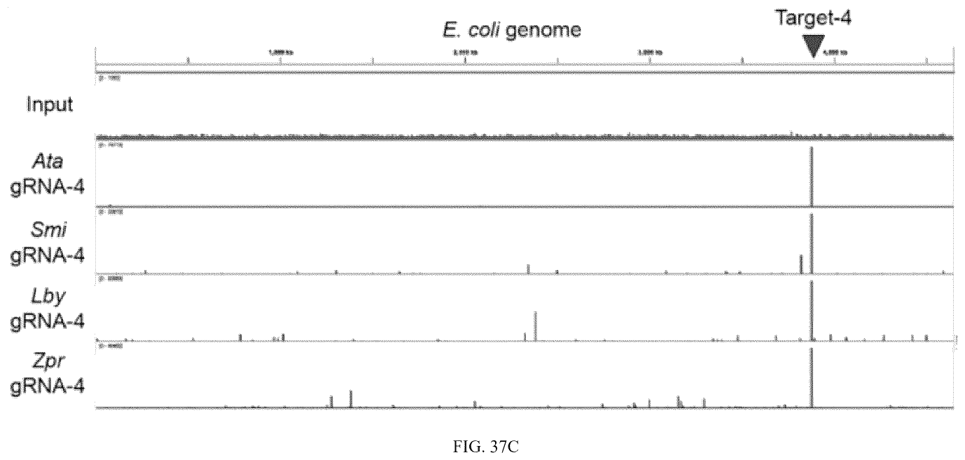

Re-programmability of gRNAs for RNA-guided DNA-targeting of dCas12f and RpoE To test whether the gRNA and TAM were correctly determined by RIP-seq and ChIP-seq, new guide sequences were cloned for one representative system (here, Ata), targeting 4 different DNA sites tiled across the E. coli K-12 genome. The native (e.g., wild-type, or WT) 14-nt guide sequence portion was replaced with a 20-nt guide sequence complementary to the genomic E. coli target, adjacent to a ‘G’ TAM. Ata dCas12f successfully targeted and bound all 4 genomic target sites, as revealed by robust ChIP-seq enrichment (FIG. 37A). Next, to test whether the sigma factor RpoE is targeted to the same loci by forming a co-complex with dCas12f, the 3×FLAG tag was moved from dCas12f to the N-terminus of RpoE. Then, ChIP-seq was performed using the same protocol, except for now focusing on DNA sites in the E. coli genome bound by RpoE. Strikingly, RpoE showed distinct enrichment at all four target sites (FIG. 37B) providing evidence for co-complex formation of RpoE and dCas12f. The four gRNAs were designed to target intergenic regions, upstream of protein-coding genes, to simultaneously test whether targeting RpoE to those sites would impact gene transcription. By applying total RNA-seq to the same four samples, the target site 4 sample showed detectable additional RNA-seq coverage not present in any of the other samples (FIG. 37C). Interestingly, target site 4 also showed the strongest dCas12f and RpoE ChIP-seq signals. In conclusion, these data provide evidence for programmable RNA-guided transcriptional activation mediated by a complex of gRNA-bound dCas12f and RpoE.

In other embodiments and experiments, three other dCas12f homologs (Smi, Lby, and Zpr) could be reprogrammed by user-defined gRNAs to target site 4 in E. coli cells (FIG. 37D), confirming that that TAM and guide sequence were correctly determined, and that these proteins are easily reprogrammable in a cellular context.

Importantly, these experiments failed to reveal any evidence of cellular toxicity, which would be expected in the case of a catalytically-active Cas12 enzyme being expressed with a genome-matching gRNA in E. coli cells. Thus, the experiments also provide evidence for these cas12f genes to indeed encode naturally catalytically deactivated Cas12f proteins that nevertheless retain the ability to target and tightly bind genomic DNA target sites matching the gRNA guide sequence.

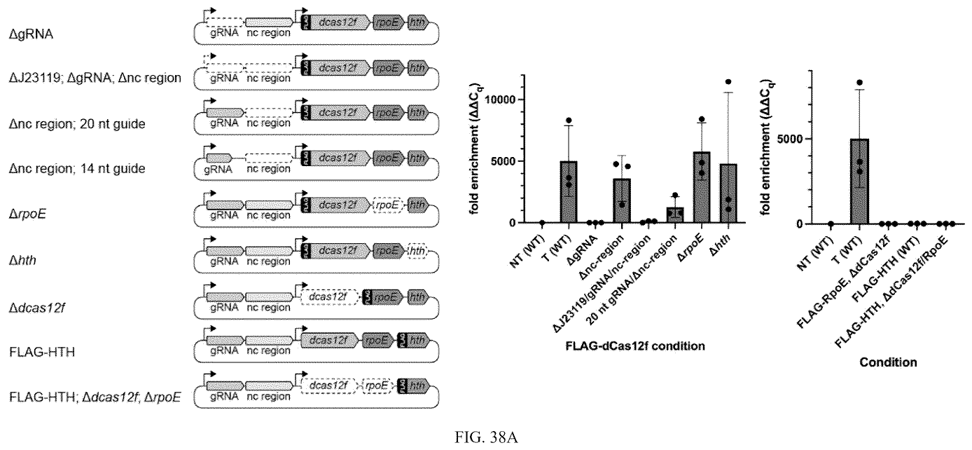

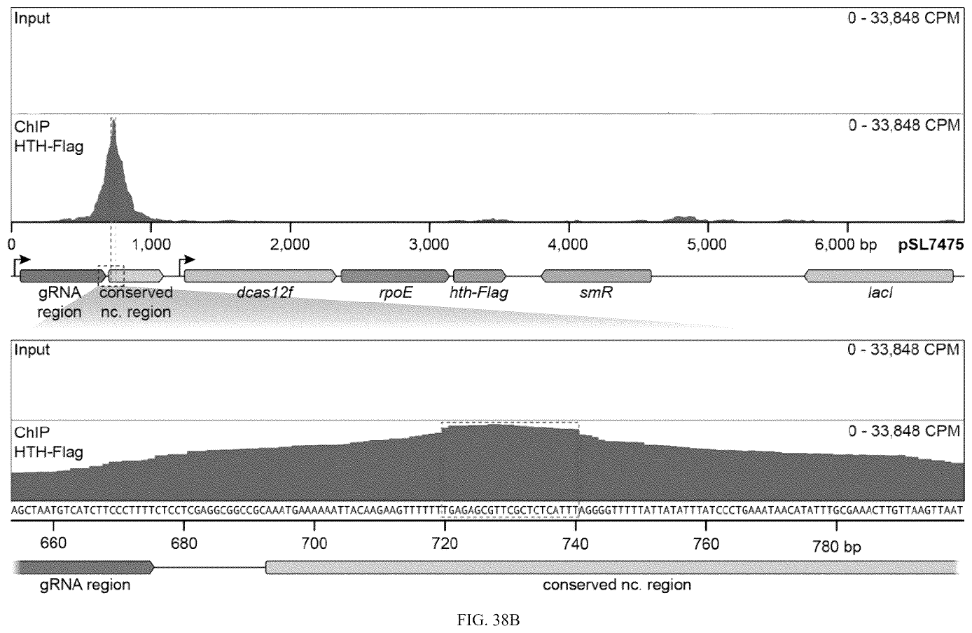

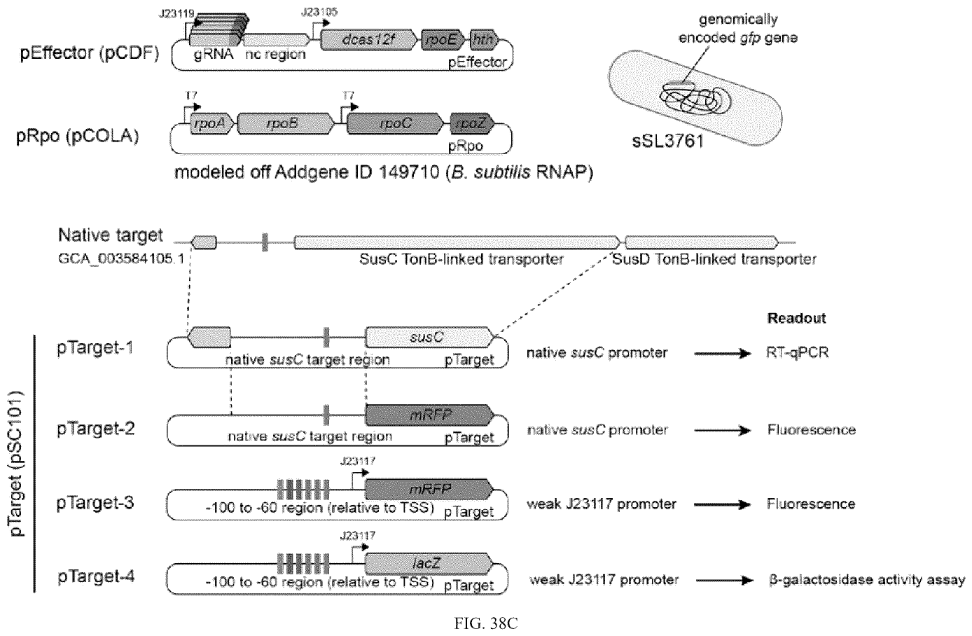

Determining protein requirements for RNA-guided DNA targeting of RpoE-associated dCas12f While ChIP-seq provided evidence for RpoE and dCas12f interacting, the role of the HTH protein remained unclear. To address this question, the Ata homolog system was chosen and components were deleted systematically from the expression plasmid. The extent of DNA binding at target site 4 as measured by ChIP-qPCR enrichment served as the readout for the various perturbations. Results are shown in FIG. 38A. The HTH protein was not recruited to the site targeted by dCas12f and RpoE (target site 4). Furthermore, deletion of the HTH protein-coding gene does not affect recruitment of dCas12f to the target site.

Heterologous approaches to demonstrate RNA-guided gene activation are described in FIG. 38C and include a native target site from the Ata organism, as well as tiled targets upstream of the promoter, and addition of the native RNAP from Ata, if required (FIG. 38C). Plasmids for gene activation experiments are listed in Table 2.

Genome engineering applications of dCas12f The above experimental data indicate that naturally deactivated Cas12f homologs (dCas12f), which are encoded in an operon with RpoE, function as RNA-guided DNA binding proteins capable of physical recruitment of RpoE to DNA target sites specified through RNA-DNA base-pairing interactions and recognition of a cognate TAM. The minimal size of dCas12f offers distinct promise for genome engineering applications that benefit from a compact CRISPR-associated protein, as compared to other Cas12 and Cas9 homologs, and the herein disclosed dCas12f proteins are also advantageous in their minimal requirement of a TAM sequence comprising only a single guanine nucleotide adjacent to the RNA-guided DNA target site. Thus, these proteins offer unique versatility and flexibility in targetable space within a genome of interest, because of the ubiquity of “G” TAMs with an average spacing every 2 base-pairs, when considering both strands of DNA.

A large set of CRISPR-associated technologies make use of non-cleaving variants of Cas9 or Cas12, often referred to as dCas9 or dCas12, respectively. These proteins can be fused to various functional effector domains for a wide range of applications, including but not limited to: deaminases (for base editing); reverse transcriptases (for prime editing); transcriptional activator domains (for CRISPR activation, also known as CRISPRa); transcriptional repressor domains (for CRISPR interference, also known as CRISPRi); histone and/or DNA modification domains (for epigenome editing); fluorescent proteins (for genomic locus imaging); and many more. In other embodiments, editing tools are generated by fusing similar domains to the dCas12f proteins described in this work, to achieve user-defined engineering end-goals but with a far more compact RNA-guided DNA targeting proteins. These applications with dCas12f benefit from the compact coding size of the fusion construct, such that desired tools can be encoded within a single viral vector, or delivered at higher dosage using non-viral lipid nanoparticle (LNP) formulations, given the smaller size of the protein and/or RNA components.

In other embodiments, effector domains are fused directly to the RpoE protein, allowing for natural complex formation between the dCas12f protein and the RpoE protein fused to the editing reagent of interest. With this approach, additional control can be achieved by regulating the binding and assembly of the complex of dCas12f and RpoE, thereby restricting the editing output to only those cellular or physiological contexts where the binding interactions takes place.

In certain bacterial embodiments, dCas12f is used with its cognate RpoE protein, to achieve targeted gene activation using RNA-guided DNA targeting and guide RNAs targeted to specific regions upstream of target genes of interest. In this approach, a gene that is normally lowly expressed can be amplified in expression level, through dCas12f-mediated targeting of activation domains directly to a locus of interest, thus leading to local RNA polymerase (RNAP) recruitment to initiate transcription initiation of the gene(s) of interest.

### Example 10

### TnpB-Transposase Fusion Sequences, Genomic Accessions, and Genetic Coordinates

TnpB proteins are RNA-guided nucleases encoded in diverse insertion sequences (e.g., IS200/IS605 and IS607 superfamily), and are ancestral to Cas12 CRISPR RNA-guided nucleases. Evolutionary offshoots of TnpB include naturally-occurring, nuclease dead Cas12 homologs that are capable of programmable DNA-cargo transposition, in concert with other transposition proteins (e.g., TnsB, TnsC, and TniQ) (Cas12k from CRISPR-associated transposon or CAST systems). While Cas12k proteins are large polypeptides, raising potential challenges in delivering these ribonucleoprotein complexes for therapeutic applications, TnpB proteins are compact effectors that may alleviate delivery size constraints. Additionally, Cas12k-mediated recruitment of multiple transposition proteins is one potential barrier to efficient genomic modification in eukaryotic organisms. Here, fusions of TnpB and transposase proteins were identified that serve as platforms for programmable, RNA-guided genome modification.

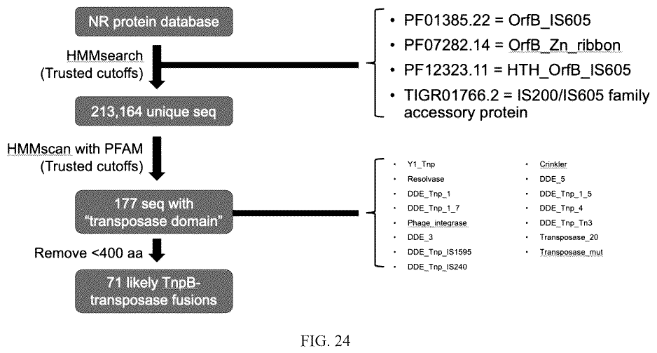

Bioinformatic identification of TnpB-transposase fusion proteins A bioinformatic pipeline was developed to identify TnpB proteins that are genetically fused to transposase domains (FIG. 24). Profile hidden Markov models (HMMs) [using PFAM: PF01385.22, PF07282.14, PF12323.11 and TIGRFAM: TIGR01766.2] were used to search the NCBI non-redundant (NR) protein database with the trusted cutoff threshold (--cut_tc) in HMMER, resulting in the identification of 213, 164 unique proteins with TnpB-like domains. These TnpB-like proteins were then scanned with the PFAM database (vA_2021-11-15) in HMMER (--cut_tc) to annotate any additional domains identifiable in their primary sequences. 1,605 TnpB-like fusion proteins were identified, representing fusions of TnpB domains to 560 unique domains. Fifteen profile HMMs were manually selected as transposase-related domains (shown in FIG. 24), and 177 sequences containing both TnpB and the selected transposase domains were retrieved from the NR database. Since TnpB proteins are ˜300-400 amino acids in length, proteins less than 400 amino acids long were removed from the set of 177 fusions, resulting in a dataset of 71 TnpB-transposase fusion proteins.

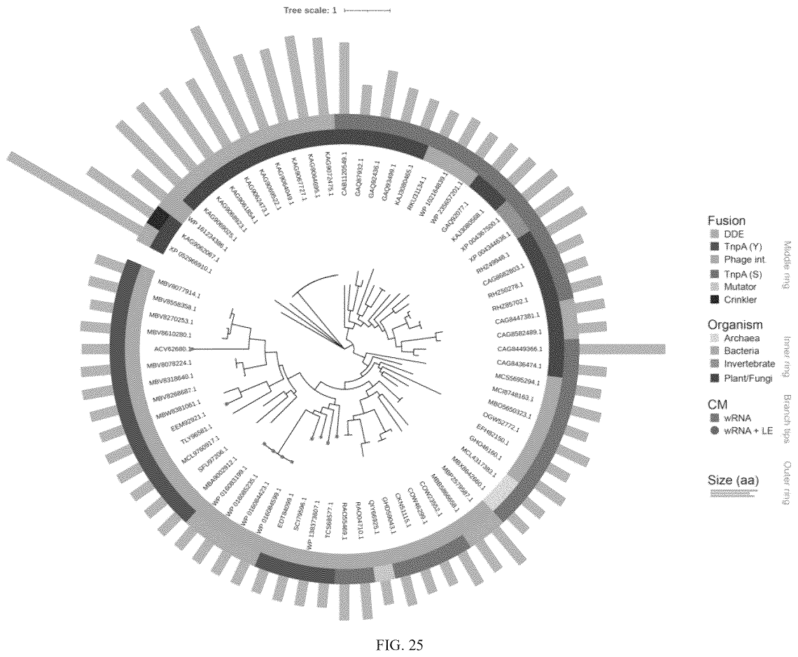

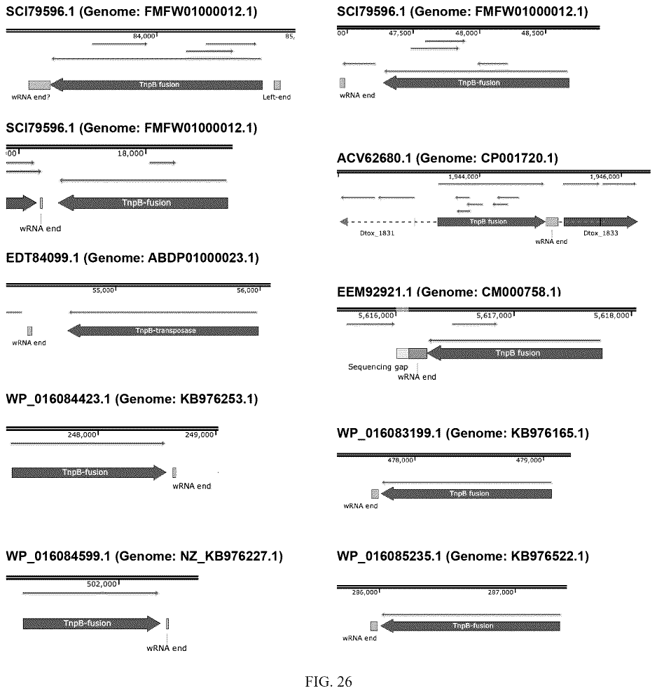

MAFFT (with the LINSI option) was used to align the TnpB-transposase fusion proteins, and a phylogenetic tree was built in FastTree (-wag-gamma options). Genomic sequences and taxonomic information for each TnpB-transposase fusion were retrieved from NCBI using the batch-entrez tool. Taxonomy, protein size, and transposase domains detected by HMMER were used to annotate the phylogenetic tree (FIG. 25), revealing fusions of transposase domains to bacterial and archaeal TnpB proteins, in addition to eukaryotic TnpB homologs (e.g., Fanzors).

TnpB proteins utilize ωRNAs (OMEGA-RNAs) comprised of a scaffold and guide sequence to direct RuvC-mediated DNA cleavage. Genetic loci encoding TnpB/Fanzor-transposase (hereinafter, TnpB-transposase) fusion proteins, including 500 base pairs upstream and downstream of the protein coding gene, were extracted with the Biostrings package in R. Sequence covariation models described in previous work (Meers, C. et al. bioRxiv 2023.03.14.532601 (2023) doi: 10.1101/2023.03.14.532601) were used to define the boundaries of ωRNA scaffolds via the CMsearch function of INFERNAL (cutoff: e-value <1e-7). This approach resulted in the identification of ωRNA scaffolds for 10 loci encoding TnpB-transposase fusions (FIGS. 25 and 26), indicating that these proteins utilize a similar ωRNA-guided targeting mechanism to standard, unfused, TnpB proteins.

TnpB proteins are encoded in diverse insertion sequence elements (e.g., IS200/IS605 and IS607 superfamily), many of which have conserved sequences or secondary structures in the left end (LE) of the element that are recognized during the excision phase of transposition. Excision at the right end (RE) of the element occurs at the scaffold-guide boundary of the ωRNA sequence. An additional covariation model built from the LE sequences of G. stearothermophilus IS200/IS605 superfamily elements (described in Meers, C. et al. bioRxiv 2023.03.14.532601 (2023)) was used to search TnpB-transposase fusion loci via the CMsearch function of INFERNAL (cutoff: e-value <1e-8), resulting in the identification of LE sequences for one TnpB-transposase (FIGS. 25 and 26). The boundaries of the LE and RE (e.g., ωRNA scaffold-guide boundary) sequences of this fusion locus indicate that the TnpB-transposase protein-coding gene is the sole open reading frame in this element, indicating that transposition of this element is not catalyzed by another gene product contained within the element.

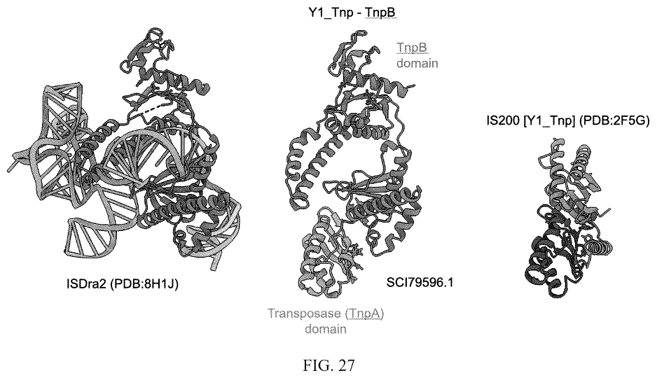

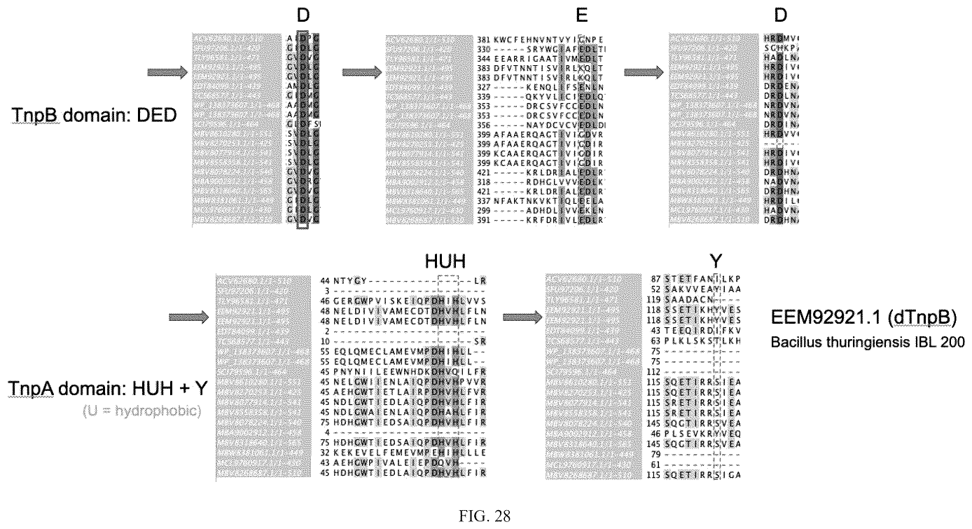

Structural predictions built with AlphaFold (v2.3), indicate that these fusion proteins have the signature folds of transposase and TnpB domains (example shown in FIG. 27). Additional analyses of multiple sequence alignments of TnpB-transposase sequences, guided by these structural predictions, indicated that these fusions containing TnpB and transposase residues are expected to facilitate the respective catalytic activities of each domain (e.g., nuclease and transposition activities) (example shown in FIG. 28).

Utilization of dTnpB for genome targeting and modification applications Natural TnpB-transposase fusion proteins represent a new and adaptable structural platform for programmable RNA-guided transposition. By changing the sequence of ωRNA guides, transposition of large DNA cargoes can be targeted to specific genetic addresses. In one embodiment, TnpB-transposase fusion proteins mobilize DNA constructs flanked by insertion element right end and left end sequences, and direct transposition of the intervening sequence to a specific sequence in the genome of a bacterium, archaeaon, or eukaryote, or to a non-genomic element (e.g., plasmid, bacterial artificial chromosome). A nuclear localization signal (NLS) may be included, and may be encoded at the N-terminus, C-terminus, or internally. In this embodiment, the naturally occurring genetic fusion of an RNA-guided DNA binding protein to a DNA transposase results in co-localization of the targeting and transposition proteins, resulting in robust DNA cargo insertion efficiencies.

### Materials and Methods

Bioinformatic identification of natural, nuclease-dead TnpB homologs (TldRs). An initial search of the NCBI non-redundant (NR) protein database, queried with TnpB sequences from H. pylori and G. stearothermophilus (WP_078217163.1 and WP_047817673.1, respectively) in Jackhmmer, resulted in the identification of 95,731 unique TnpB-like proteins, which were further clustered at 50% amino acid identity (across 50% sequence coverage) via CD-HIT to produce a set of 2,646 representative TnpB sequences. A multiple sequence alignment (MSA) was then constructed with MAFFT (EINSI; four rounds), which was trimmed manually with trimAl (90% gap threshold; v1.4.rev15). The resulting alignment of TnpB/TldR homologs was used to construct a phylogenetic tree in IQTree (WAG model, 1000 replicates for SH-aLRT, aBayes, and ultrafast bootstrap), which was annotated and visualized in ITOL.

To assess the conservation of RuvC catalytic residues in each TnpB protein sequence, each sequence in the MSA was compared to structurally characterized orthologs (e.g., DraTnpB from ISDra2 and Cas12f; PDB ID 8H1J and 7L48, respectively). This comparison was performed by aligning each candidate, as well as the homologs represented in the closest five tree branches on either side of it, to DraTnpB and UnCas12f using the AlignSeqs function of the DECIPHER package in R. TnpB-like protein sequences with less than two conserved residues of the RuvC DED catalytic motif were extracted using the Biostrings package in R. For each sequence with less than two active site residues identified (defined as a TnpB-like nuclease-dead Repressor, or TldR), related homologs were retrieved from initial sequence clusters, and additional related homologs were identified via BLASTP searches of the NR protein database (e-value <1e-50, query coverage >80%, max target sequences=50). Each representative sequence and all of their cluster members were used as queries in these BLASTP searches, and the active sites from BLAST hits were checked by aligning proteins to structurally determined representatives, as described above. This approach resulted in the identification of 494 TldR homologs. Genomes encoding each TldR were retrieved from NCBI using the batch-entrez tool. TldR-encoding loci (e.g., tldR+/−20 kbp) were extracted using the Biostrings package in R, and each tldR locus was annotated with Eggnog (-m diamond--evalue 0.001--score 60--pident 40--query_cover 20--subject_cover 20--genepred prodigal--go_evidence non-electronic-- pfam_realign none). Annotated tldR loci were manually inspected in Geneious.

Bioinformatic analyses of fliC_{P}-, oppf-, and csrA-associated TldR homologs. To further investigate fliC-associated TldR homologs, cluster members were extracted for three representative branches in the tree shown in FIG. 1 (WP_193971683.1, WP_064735610.1, and WP_048785942.1). The protein file representing these combined clusters was supplemented with additional homologs identified via BLASTP searches of the NR database. The resulting concatenated protein file included both TldR and related TnpB sequences. To increase the diversity of TnpB proteins represented in this dataset, three additional TnpB homologs (WP_269608765.1, WP_024186316.1, WP_059759460.1) were identified and manually added to this protein file via web-based BLASTP searches queried with the TnpB protein sequences already present in the dataset (e-value <0.05). An MSA was constructed from these sequences and DraTnpB using the AlignSeqs function of the DECIPHER package in R to verify the active site composition of each ortholog. To determine which tldR tnpB genes were associated with fliC, Eggnog annotation information was analyzed for each locus (described above) and TldR/TnpB sequences that were encoded within three open reading frames of fliC were extracted.

A locus was defined as phage-associated if it contained four or more gene annotations that contained the word “Phage”, “phage”, “Viridae”, or “viridae”. TldR/TnpB protein sequences were then de-duplicated via CD-HIT (-c 1.0), and an MSA was built in MAFFT (LINSI) from the resulting set of 160 unique proteins. Protein domain coordinates displayed around the tree in FIG. 2C were inferred by cross-referencing the MSA and predicted structures. The phylogenetic tree shown in FIG. 2C was built from the TldR/TnpB MSA in FastTree (-wag-gamma) and was annotated and visualized in ITOL. Structural models of each candidate shown in FIG. 1D were predicted with AlphaFold (v2.3) and displayed with ChimeraX (v1.6); MSAs were visualized in Jalview.

To interrogate oppF-associated TldR sequences, cluster members and additional homologs identified via BLASTP searches of the NR database (e-value <1e-50, query coverage >80%, max target sequences=50) for six branches representing TldR proteins in the FIG. 1C tree (RBR34854.1, WP_016173224.1, WP_156233666.1, NTQ19983.1, OTP13636.1, OSH30650.1) were extracted. These sequences were concatenated with cluster members and additional homologs identified through an identical BLASTP search of one representative TnpB branch (EOH94253.1) that corresponded to the closest branch to the six TldR branches in the tree. To increase the diversity of related TnpB proteins represented in this dataset, three additional TnpB homologs (WP_242450195.1, WP_028983493.1, WP_277281207.1) were identified and manually added to this protein file via web-based BLASTP searches queried with the TnpB protein sequences already present in the dataset (e-value <0.05). Genomes encoding TldR/TnpB proteins were downloaded from NCBI using the Batch-entrez tool, relevant loci (tldR tnpB+/−20 kbp) were extracted using the Biostrings package in R, and each locus was annotated with Eggnog (see above). Each TldR/TnpB protein was individually aligned to DraTnpB using the AlignSeqs function of the DECIPHER package in R to verify its RuvC active site composition. TldR/TnpB sequences were then deduplicated via CD-HIT (−c 1.0), and an MSA was built in MAFFT (LINSI) from the resulting set of 204 unique proteins. An initial phylogenetic tree was constructed in FastTree (-wag-gamma), and this tree was used to guide the selection of eight representative TldRs and four representative TnpBs (shown in FIG. 19) that were structurally predicted with ColabFold (v1.5). These twelve predicted structures were used to guide an alignment of TldR/TnpB protein sequences in Promals3D, and the resulting MSA was used to build the tree in FIG. 6 in FastTree (-wag-gamma). Protein domain coordinates displayed around the tree in FIG. 6 were inferred by cross referencing the MSA and predicted structures. The phylogenetic tree was annotated and visualized in ITOL.

To probe oppF-associated TldR loci, cluster members and additional homologs identified via BLASTP searches of the NR database (e-value <1e-50, query coverage >80%, max target sequences=500) for one TldR protein in the FIG. 1C tree (WP_204886977.1) were extracted. Genomes encoding TldR/TnpB proteins were downloaded from NCBI using the Batch-entrez tool, relevant loci (tldR tnpB+/−20 kbp) were extracted using the Biostrings package in R, and each locus was annotated with Eggnog (see above). Each TldR/TnpB protein was individually aligned to DraTnpB using the AlignSeqs function of the DECIPHER package in R to verify its RuvC active site composition. TldR/TnpB sequences were then deduplicated via CD-HIT (-c 1.0), resulting in 41 unique TldR proteins.

Bioinformatic identification of TldR-associated gRNA sequences. To define the boundaries of gRNA scaffolds in fliC_{P}-tldR loci, a general gRNA covariance model (CM) described in Meers, C. et al. (Nature 622, 863-871 (2023)) was used. The CMsearch function of Infernal (Inference of RNA alignments; v1.1.2) was used to scan nucleotide sequences of tldR and 500-bp flanking windows, resulting in the identification of putative gRNA scaffold sequences. These TldR-associated gRNA scaffold boundaries were confirmed by comparing fliC_{P}-tldR loci to ωRNAs from confidently predicted annotations of catalytically active TnpB loci. Putative TldR guide sequences could then be retrieved from the 3′ boundary of putative gRNA scaffolds, enabling prediction of native fliC_{P}-associated TldR targets. Putative guides are listed in the sequence tables below).

An analogous search of oppF-associated tldR loci with a general gRNA CM failed to identify putative gRNA sequences. For this group of tldR loci, a new CM was built from ωRNA sequences associated more closely related TnpB loci. Using the comparative genomics strategy outlined in FIG. 3A, the putative transposon right end (RE) was manually identified for one TnpB-encoding IS element (WP_113785139.1 in KZ845747). The nucleotide sequences for all the related tnpB genes and 500 bp of sequence downstream of tldR were aligned with MAFFT (LINSI). The resulting alignment was trimmed at the 3′ end to the position of the ωRNA scaffold-guide boundary identified for the WP_113785139.1 locus. This putative set of TnpB ωRNA sequences was used realigned with LocaRNA (--max-diff-at-am=25--max-diff=60--min-prob=0.01--indel=−50--indel-opening=−750--plfold-span=100--alifold-consensus-dp; v2.0.0), and a CM (ABC_gRNA_v1) was built and calibrated with Infernal. The CMsearch function of Infernal was then used to search sequences composed of tldR tnpB and 500 bp of downstream sequence with the ABC_gRNA_v1 CM. This search resulted in gRNA identification for some, but not all, tldR loci. Thus, a second gRNA CM was built by extracting the newly identified TldR/TnpB gRNA sequences from their respective genomes, merging them with the sequences used to construct ABC_gRNA_v1, aligning the prospective gRNA dataset in LocaRNA, and building and calibrating a new CM with Infernal (ABC_gRNA_v2). When sequences comprising tldR tnpB and 500 bp downstream were scanned with the ABC_gRNA_v2 CM, via CMsearch, putative gRNA sequences were identified for the remaining tldR loci (listed in the sequence tables below).

Visualization of RNA-seq data from the NCBI short read archive (SRA) and gene expression omnibus (GEO)). To assess gRNA expression from a representative fliC_{P}-tldR locus, an RNA-seq dataset was downloaded from the NCBI SRA (accession: ERR6044061). Reads were aligned to the Enterobacter cloacae AR_154 genome (CP029716.1) with using bwa-mem2 (v2.2.1) in paired-end mode with default parameters, and alignments were converted to BAM files with SAMtools. Bigwig files were generated with the bamCoverage utility in deepTools, and unique reads mapping to the forward strand were visualized with the Integrated Genome Viewer (IGV). Expression of gRNA and oppA from an oppf-tldR locus was assessed by downloading an RNA-seq analysis from the NCBI GEO (accession: GSE115009). Normalized coverage files (ID-005241, ID-005244, ID-005245, ID-005246) for the forward strand were visualized in IGV.

Plasmid and E. coli strain construction. All strains and plasmids used in this study are described in Tables 1 and 2, respectively, and a subset is available from Addgene. In brief, genes encoding candidate TldR and TnpB homologs (Table 3), alongside their putative gRNAs, were synthesized by GenScript and subcloned into the Pfol and Bsu36i restriction sites of pCDFDuet-1, to generate pEffector, similar to Meers, C. et al. (2023). Expression vectors contained constitutive J23105 and J23119 promoters driving expression of tldR/tnpB and the gRNA, respectively, and tldR/tnpB genes encoded an appended 3×FLAG-tag at the N-terminus. gRNAs for fliC_{P}-associated TldRs were designed to target the host fliC 5′ UTR site, whereas gRNAs of oppF-associated TldRs were engineered to target the genomic site natively targeted by a Gs/TnpB3 homolog. Derivatives of these pEffector plasmids, or their associated pTarget plasmids (for plasmid interference assays), were cloned using a combination of methods, including Gibson assembly, restriction digestion-ligation, ligation of hybridized oligonucleotides, and around-the-horn PCR. Plasmids were cloned, propagated in NEB Turbo cells (NEB), purified using Miniprep Kits (Qiagen), and verified by Sanger sequencing (GENEWIZ).

A custom E. coli K12 MG1655 strain that contained genomically-encoded sfGFP and mRIP genes was constructed by adding three target sites adjacent to bioinformatically predicted TAM sequences upstream of the mRFP ORF, in between the constitutive promoter driving RFP expression and the corresponding ribosome binding site (sSL3580; derivative of GenBank: NC_000913.3) (Table 1). The original strain (with genomic sfGFP and mRFP) was a gift from L. S. Qi. The inserted target sites represent 25-bp sequences derived from the 5′ UTR of host fliC (Enterobacter cloacae complex sp. strain AR_0154; GenBank: CP029716.1), an ABC transporter gene (Enterococcus faecium strain BP657; GenBank: CP059816.1), and a GstTnpB3 native target used in Meers, C. et al. (2023).

Chromatin immunoprecipitation sequencing (ChIP-seq) and motif analyses of genomic sites bound by TldR. ChIP-seq experiments and data analyses were generally performed as described previously (Meers, C. et al. (2023) and Hoffmann, F. T. et al. Nature 609, 384-393 (2022)), except for the use of sSL3580. In brief, E. coli MG1655 cells were transformed with pEffector and incubated for 16 h at 37° C. on LB-agar plates with antibiotic (200 μg ml⁻¹ spectinomycin). Cells were scraped and resuspended in LB broth. The OD₆₀₀ was measured, and approximately 4.0×10⁸ cells (equivalent to 1 ml with an OD₆₀₀ of 0.25) were spread onto two LB-agar plates containing antibiotic (200 μg ml⁻¹ spectinomycin). Plates were incubated at 37° C. for 24 h. All cell material from both plates was then scraped and transferred to a 50-ml conical tube. Cross-linking was performed in LB medium using formaldehyde (37% solution; Thermo Fisher Scientific) and was quenched using glycine, followed by two washes in TBS buffer (20 mM Tris-HCl pH 7.5, 0.15 M NaCl). Cells were pelleted and flash-frozen using liquid nitrogen and stored at −80° C.

Chromatin immunoprecipitation of FLAG-tagged TnpB and TldR proteins was performed using Dynabeads Protein G (Thermo Fisher Scientific) slurry (hereafter, beads or magnetic beads) conjugated to ANTI-FLAG M2 antibodies produced in mouse (Sigma-Aldrich). Samples were sonicated on a M220 Focused-ultrasonicator (Covaris) with the following SonoLab 7.2 settings: minimum temperature, 4° C.; set point, 6° C.; maximum temperature, 8° C.; peak power, 75.0; duty factor, 10; cycles/bursts, 200; 17.5 min sonication time. After sonication, a non-immunoprecipitated input control sample was frozen. The remainder of the cleared sonication lysate was incubated overnight with anti-FLAG-conjugated magnetic beads. The next day, beads were washed, and protein-DNA complexes were eluted. The non-immunoprecipitated input samples were thawed, and both immunoprecipitated and non-immunoprecipitated controls were incubated at 65° C. overnight to reverse-crosslink proteins and DNA. The next day, samples were treated with RNase A (Thermo Fisher Scientific) followed by Proteinase K (Thermo Fisher Scientific) and purified using QIAquick spin columns (QIAGEN).

ChIP-seq Illumina libraries were prepared for immunoprecipitated and input samples using the NEBNext Ultra II DNA Library Prep Kit for Illumina (NEB). Following adapter ligation, Illumina barcodes were added by PCR amplification (12 cycles). ˜450-bp DNA fragments were selected using two-sided AMPure XP bead (Beckman Coulter) size selection. DNA concentrations were determined using the DeNovix dsDNA Ultra High Sensitivity Kit and dsDNA High Sensitivity Kit. Illumina libraries were sequenced in paired-end mode on the Illumina NextSeq platform, with automated demultiplexing and adapter trimming (Illumina). >2,000,000 raw reads, including genomic- and plasmid-mapping reads, were obtained for each ChIP-seq sample.

Following sequencing, paired-end reads were trimmed and mapped to a custom E. coli K12 MG1655 reference genome (derivative of GenBank: NC_000913.3). Genomic lacZ and lacl regions partially identical to plasmid-encoded genes were masked in all alignments (genomic coordinates: 366,386-367,588). Mapped reads were sorted and indexed, and multi-mapping reads were excluded. Alignments were normalized by counts per million (CPM) and converted to 1-bp-bin bigwig files using the deepTools2 command bamCoverage, with the following parameters:--normalizeUsing CPM-bs 1. CPM-normalized reads were visualized in IGV. Genome-wide views were generated using plots of maximum read coverage values in 1-kb bins. Peak calling was performed using MACS3 (version 3.0.0a7) using the non-immunoprecipitated control sample of EcoTldR as reference. 200-bp sequences for each peak were extracted from the E. coli reference genome using BEDTools (v2.30.0), and sequence motifs were identified using MEME-ChIP (5.4.1).

RNA immunoprecipitation sequencing (RIP-seq) of RNA bound by TldR. Cells harvested for RIP-seq were cultured as described for ChIP-seq using an E. coli K12 MG1655 strain expressing sfGFP and mRFP (sSL3580). Colonies from a single plate were scraped and resuspended in 1 ml of TBS buffer (20 mM Tris-HCl pH 7.5, 0.15 M NaCl). Next, the OD₆₀₀ was measured for a 1:20 mixture of the cell suspension and TBS buffer, and a standardized amount of cell material equivalent to 20 ml of OD₆₀₀=0.5 was aliquoted. Cells were pelleted by centrifugation at 4,000 g and 4° C. for 5 min. The supernatant was discarded, and pellets were stored at −80° C.

Antibodies for immunoprecipitation were conjugated to magnetic beads as follows: for each sample, 60 μl Dynabeads Protein G (Thermo Fisher Scientific) were washed 3× in 1 ml RIP lysis buffer (20 mM Tris-HCl pH 7.5, 150 mM KCl, 1 mM MgCl₂, 0.2% Triton X-100), resuspended in 1 ml RIP lysis buffer, and combined with 20 μl anti-FLAG M2 antibody (Sigma-Aldrich), and rotated for >3 h at 4° C. Antibody-bead complexes were washed 3× to remove unconjugated antibodies, and resuspended in 60 μl RIP lysis buffer per sample.

Flash-frozen cell pellets were resuspended in 1.2 ml RIP lysis buffer supplemented with complete Protease Inhibitor Cocktail (Roche) and SUPERase⋅In RNase Inhibitor (Thermo Fisher Scientific). Cells were then sonicated for 1.5 min total (2 sec ON, 5 sec OFF) at 20% amplitude. Lysates were centrifuged for 15 min at 4° C. at 21,000 g to pellet cell debris and insoluble material, and the supernatant was transferred to a new tube. At this point, a small volume of each sample (24 μl, or 2%) was set aside as the “input” starting material and stored at −80° C.

For immunoprecipitation, each sample was combined with 60 μl antibody-bead complex and rotated overnight at 4° C. Next, each sample was washed 3× with ice-cold RIP wash buffer (20 mM Tris-HCl, 150 mM KCl, 1 mM MgCl₂). After the last wash, beads were resuspended in 1 ml TRIzol (Thermo Fisher Scientific) and RNA was eluted from the beads by incubating at RT for 5 min. A magnetic rack was used to separate beads from the supernatant, which was transferred to a new tube and combined with 200 μl chloroform. Each sample was mixed vigorously by inversion, incubated at RT for 3 min, and centrifuged for 15 min at 4° C. at 12,000 g. RNA was isolated from the upper aqueous phase using the RNA Clean & Concentrator-5 kit (Zymo Research). RNA from input samples was isolated in the same manner using TRIzol and column purification. High-throughput sequencing library preparation was performed as described below for total RNA-seq of Enterobacter strains. Libraries were sequenced on an Illumina NextSeq 550 in paired-end mode with 75 cycles per end.

Adapter trimming, quality trimming, and read length filtering of RIP-seq reads was performed as described below for total RNA-seq experiments. Trimmed and filtered reads were mapped to a reference containing both the MG1655 genome (NC_000913.3) and plasmid sequences using bwa-mem2 v2.2.1, with default parameters. Mapped reads were sorted, indexed, and converted into coverage tracks as described below for total RNA-seq experiments.

Plasmid cleavage assays. Plasmid interference assays were generally performed as previously described in Meers, C. et al. (2023). E. coli K12 MG1655 (sSL0810) cells were transformed with pTarget plasmids (vector sequences are listed in Table 2), and single colony isolates were selected to prepare chemically competent cells. Next, cells were transformed with 400 ng of pEffector plasmid or empty vector. After 3 h recovery at 37° C., cells were pelleted by centrifugation at 4,000 g for 5 min and resuspended in 100 μl of H²O. Cells were then serially diluted (10×), plated as 8 μl spots onto LB agar supplemented with spectinomycin (200 μg ml⁻¹) and kanamycin (50 μg ml⁻¹), and grown for 16 h at 37° C. Plate images were taken using a BioRad Gel Doc XR+ imager.

Plasmid interference assays were quantified by determining the number of colony-forming units (CFU) following transformation. Experiments were performed as described above, however for each experiment, 30 μl of a 10-fold dilution were plated onto a full LB agar plate containing spectinomycin (200 μg ml⁻¹) and kanamycin (50 μg ml⁻¹). CFUs were counted following 16 h of growth at 37° C. and reported as CFUs per μg of transformed pEffector plasmid.

RFP repression assays. The RFP repression assay protocol was adapted from previous studies (Meers, C. et al. (2023) and Hoffmann, F. T. et al. (2022)). An E. coli strain expressing a genomically-integrated sfGFP (sSL3761), derived from a strain kindly provided by L. S. Qi (Cell 152, 1173-1183 (2013)), was co-transformed with 200 ng of pEffector and pTarget (vector sequences listed in Table 2). Protein components and guide RNAs (gRNA, sgRNA or crRNA) were constitutively expressed from pEffector. pTargets were cloned to encode an mRFP gene under the control of a constitutive promoter. For RFP repression assays shown in FIG. 4G, gRNAs were designed to target the constitutive RFP promoter on either strand, and 5-bp TAM sequences were inserted 5′ of each target site. For RFP repression assays shown in FIG. 4H, 25-bp sequences containing the TAM/PAM and target site in either orientation were inserted in between the mRFP promoter and ribosome binding site.

Transformed cells were plated on LB-agar with antibiotic selection, and at least three of the resulting colonies on each plate were used to inoculate overnight liquid cultures. For each sample, 1 μl of the overnight culture was used to inoculate 200 μl of LB medium on a 96-well optical-bottom plate. The fluorescence signals for sfGFP and mRFP were measured alongside the OD₆₀₀ using a Synergy Neo2 microplate reader (Biotek), while shaking at 37° C. for 16 h. For all samples, the fluorescence intensities at OD₆₀₀=1.0 were used to determine the fold repression for each TldR or Cas targeting complex, and the data were normalized to the non-repressed signal for sSL3761. Background GFP and RFP fluorescence intensities at OD₆₀₀=1.0 were determined using an E. coli K12 MG1655 strain (sSL0810) lacking sfGFP and mRFP genes, and were subtracted from all RFP and GFP fluorescence measurements.

Total RNA sequencing of Enterobacter strains. Enterobacter cloacae strains (sSL3710, sSL3711, and sSL3712) were obtained from a CDC isolate panel (Enterobacterales Carbapenemase Diversity; CRE in ARIsolateBank), and an Enterobacter sp. BIDMC93 strain (sSL3690) was kindly provided by Ashlee M. Earl at the Broad Institute; strain information is listed in Table 1. Biological replicates were obtained by isolating 3 individual clones of each Enterobacter strain on LB-agar plates and using these to inoculate overnight cultures in liquid LB media. All strains were grown at 37° C. without antibiotics and with agitation when in liquid medium (240 rpm), in a BSL-2 environment. For total RNA-seq library preparation, RNA was purified from 2 mL of exponentially growing cultures of sSL3690, sSL3710, sSL3711, and sSL3712 since RT-qPCR analyses of fliC expression showed that the TldR-mediated was more robust in exponential than in stationary phase. RNA was extracted using TRIzol and column purification (NEB Monarch RNA cleanup kit), and samples were then individually diluted in NEBuffer 2 (NEB) and fragmented by incubating at 92° C. for 1.5 min. The fragmented RNA was simultaneously treated with RppH (NEB) and TURBO DNase (Thermo Fisher Scientific) in the presence of SUPERase. In RNase Inhibitor (Thermo Fisher Scientific), in order to remove DNA and 5′ pyrophosphate. For further end repair to enable downstream adapter ligation, the RNA was treated with T4 PNK (NEB) in 1×T4 DNA ligase buffer (NEB). Samples were column-purified using RNA Clean & Concentrator-5 (Zymo Research), and the concentration was determined using the DeNovix RNA Assay (DeNovix). Illumina adapter ligation and cDNA synthesis were performed using the NEBNext Small RNA Library Prep kit, using 100 ng of RNA per sample. High-throughput sequencing was performed on an Illumina NextSeq 550 in paired-end mode with 75 cycles per end.

RNA-seq reads were processed using cutadapt (v4.2) to remove adapter sequences, trim low-quality ends from reads, and exclude reads shorter than 15 bp. Trimmed and filtered reads were aligned to reference genomes (accessions listed in Table 1) using bwa-mem2 (v2.2.1) in paired-end mode with default parameters. SAMtools (v1.17) was used to filter for uniquely mapping reads using a MAPQ score threshold of 1, and to sort and index the unique reads. Coverage tracks were generated using bamCoverage (v3.5.1) with a bin size of 1, read extension to fragment size, and normalization by counts per million mapped reads (CPM) with exact scaling. Coverage tracks were visualized using IGV. For transcript-level quantification, the number of read pairs mapping to annotated transcripts was determined using featureCounts (v2.0.2). The resulting counts values were converted to transcripts-per-million-mapped-reads (TPM) by normalizing for transcript length and sequencing depth. For differential expression analysis between genetically engineered Enterobacter strains, the counts matrix was first filtered to remove rows with fewer than 10 reads for at least 3 samples. The filtered matrix was then processed by DESeq2 (v1.40.2) in order to determine the log 2 (fold change) for each transcript between the experimental conditions, as well as the Wald test P value adjusted for multiple comparisons using the Benjamini-Hochberg approach. Significantly differentially expressed genes were determined by applying thresholds of |log 2 (fold change) |>1 and adjusted P value <0.05.

Construction of Enterobacter BIDMC93 mutants. Enterobacter cloacae strains AR_154 and AR_163 (sSL3711 and sSL3712; respectively) are both resistant to the antibiotics commonly used for colony selection following plasmid transformation, so we proceeded with recombineering in Enterobacter sp. BIDMC93. Genomic mutants (listed in Table 1) were generated using Lambda Red recombineering. Mutants were designed to introduce a chloramphenicol resistance cassette at each disrupted locus. The chloramphenicol resistance cassette was amplified by PCR with Q5 High Fidelity DNA Polymerase (NEB), using primers that contained at least 50-bp of homology to the disrupted locus. Amplified products were resolved on a 1% agarose gel and purified by gel extraction (QIAGEN). Electrocompetent Enterobacter sp. BIDMC93 cells were prepared containing a temperature-sensitive plasmid encoding Lambda Red components under a temperature-sensitive promoter (pSIM6). Immediately prior to preparing electrocompetent cells, Lambda Red protein expression was induced by incubating cells at 42° C. for 25 min. 200-500 ng of each insert was used to transform cells via electroporation (2 kV, 200 Ω, 25 μF). Cells were recovered by shaking in 1 mL of LB media at 37° C. overnight. After recovery, cells were spread on 100 mm plates with 25 μg/mL chloramphenicol and grown at 37° C. Chloramphenicol-resistant colonies were genotyped by Sanger sequencing (GENEWIZ) to confirm the desired genomic mutation.

RT-qPCR to assess host fliC′ transcription in Enterobacter sp. BIDMC 93. 200 ng of the purified total RNA was used as an input for the reverse transcription reaction. First, total RNA was treated with 1 μl dsDNase (Thermo Fisher Scientific) in 1×dsDNase reaction buffer in a final volume of 10 μl and incubated at 37° C. for 20 min. Then, 1 μl of 10 mM dNTP, μl of 2 μM oSL14254, and 1 μl of 2 μM oSL 14280 were added for gene-specific priming (rrsA and fliC, respectively), and reactions were heated at 65° C. for 5 min. Reactions were then placed directly on ice, followed by addition of 4 μl of SSIV buffer, 1 μl 100 mM DTT, 1 μl SUPERase⋅In™ (Thermo Fisher Scientific), and 1 μl of SuperScript IV Reverse Transcriptase (200 U/μl, Thermo Fisher Scientific), followed by incubation at 53° C. for 10 min, and then incubation at 80° C. for 10 min. Quantitative PCR was performed in 10 μl reaction containing 5 μl SsoAdvanced™ Universal SYBR Green Supermix (BioRad), 1 μl H₂0, 2 μl of primer pair at 2.5 μM concentration, and 2 μl of 100-fold diluted RT product. Two primer pairs were used: oSL14254/oSL14255 was used to amplify rrsA cDNA, and oSL14279/oSL14280 was used to amplify host fliC cDNA. Reactions were prepared in 384-well clear/white PCR plates (BioRad), and measurements were performed on a CFX384 RealTime PCR Detection System (BioRad) using the following thermal cycling parameters: polymerase activation and DNA denaturation (98° C. for 2.5 min), 35 cycles of amplification (98° C. for 10 s, 62° C. for 20 s). For each sample, Cq values were normalized to that of rrsA (reference housekeeping gene). Then, the normalized Cq values were compared to the normalized Cq value of fliC in the control strain (sSL3868, knock-in of cmR downstream of tldR in BIDMC93), to obtain relative expression levels, such that a value of one is equal to that of the control and higher values indicate higher expression levels.

Data availability. Next-generation sequencing data are available in the National Center for Biotechnology Information (NCBI) Sequence Read Archive: XX (BioProject Accession: XX) and the Gene Expression Omnibus (GSE245749). The published genome used for ChIP-seq analyses was obtained from NCBI (GenBank: NC_000913.3). The published genomes used for bioinformatics analyses were obtained from NCBI.

Sequences
TldR Sequences

| SEQ ID | |
|---|---|
| NO | Amino Acid Sequence |
| 1 | MIKKQAFKFLLEPNKGQLSDFLAFAGSCRFVYNKGLALLNENYRSGKKFIGYIQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKSTIVLNNVDSVHLSSGGGGDNTYQAEEK |
| | KKLIRLNKTLTRRKKHSQNWLKTKGKIDRVKSKAARIRLDNIHKATTAICKNHAVVEVVNLMDSVSDK |
| | NDNTLSMRYEFVRQLIYKQEWLGGEVIRRESKPL |
| 2 | KDKHGWYVKFPKAVLQAGCVRDRYDLGKMDLHEQKLKEVRLIPNGDTIKLEIVCEIEIKEPTITIHEATR |
| | VTGIDIGVDNLMAIAFTSGHHPVLIKGNEIKAVNQYYNKQIAHYRSLLRTGKKDSKGIHQTKRMKRISEK |
| | RNRRVKDILHKASRKIINLCVEEGIEVIVVGNNAGWKKRIHMGKKNNQTFVQIPFRTLIEMIKYKGEAAG |
| | IRVVVCEEAIQSKASSIDEDQIPVYGNDVAHTFTGKRIKRGLYRSKWHSNECRYQWSKQYHTKSISMYA |
| | RARAME |
| 3 | MLLFAGACRFVYNKGLALLKESYESGQKHMHYNQLAPLLVEWKSDPALSWLKQAPSQSLQQSLRDLD |
| | KAFSNFFYGKAEHPRFKKKGQHDAFRFPSQRVKVDQEKQLVLLPKLGWVKYRKSRNITGAIKNVSISGK |
| | LGNWYISFNTQTDIAEPIHPAISKIGVYVDTKKNITLSDGTQYIPPQSLITLPKQIQRLTNCLRKKNRYSNN |
| | WLKSKHRINRLSSRLNQVKVDYLHKASTAISKNHAMIVIADFEKKSFSADKQQKNLTTCEKSTSIHYELI |
| | RQLTYKQEWHGGLVIKLSAEKNVDAESAWTKACNLLAAGLAVTACGGEVSKDSPMKQEP |
| 4 | MVLKHKAYKFRIYPTKEQEILIAKTIRCSRFVFNHFLSKWDETYKTTGKGLSYGSCSKEIPLLKQEFDWL |
| | KEVDSTSVQMSVKHLADAFDRFFNKQNKRPRFKSKRHPVQSYKTNVQGKNQLPEVSIFGNKLKLPKLK |
| | WVRFAHSKQITGRILNATIRRNASGKYFVSLLVEHAILSDGTVYKNDRYFRLLEKKLVREQRKLSRRQRI |
| | ALNKKVKLSEARNYQKQKQRVARIHEKIANGYRPRSSKTTTSSGLRPCR |
| 5 | MRTVEFKLSLNRYQQAKVDSWLAIQRWIWNQGLHLLEEFNSFSTWDKVSQTWVPCCPIPWTYYRDSVG |
| | QLIAFTRIAKKKPYRMSCPIPQVYPKPVLESPTFFGLLYYFAQKNHSDKPWFCDVPCRFVAGTLKSLADA |
| | WTAYKSGKRQRPRYKQYKDKFRTLINNNAKPIKISGKRITLPKLGKVTVKTLDRRWLKSVPIVTLKIVKE |
| | PSGYYLQLSGCFPVNKEKPTNKAVGVSLGYSHLTTDGEKVVEPPNFYRKMEKQLVQLQRQLCRQQKTC |
| | PISTYNPSLGEHFLSCPIDPGKGANRAKTQRKISRLYEKIRRSRLATNHKISTYLVREYDAIAIVKPEIKRIT |
| | RKPIAIVNKLGEFEHNGANHKAEFSKGLLDNSLGQLAGLIKQKASVQGRELISVSPKDLPDELKQCTEKR |
| | REQLQWSRAVYSTNFSRRYRAWEWELTPGESTETLNQEPPQGGLSCDAGTTSNFILESIGLCGVGDIPETI |
| | PLLQNQSEANSSY |
| 6 | MLLFAGACRFVYNKGLALLKESYESGQKHMHYNQLAPLLVEWKSDPALSWLKQAPSQSLQQSLRDLD |
| | KAFSNFFYGNAEHPRFKKKGQHDAFRFPSQRVKVDQEKQLVLLPKLGWVKYRKSRDITGDIKNVSISGK |
| | LGKWYISFNTQTDIEEPVHPAISKIGVYVDAKKNITLSDGTQYIPPQSLITLPKQIQRLTNCLRKKNRYSNN |
| | WLKSKHRINRLSSRLNQVKVDYLHKASTAISKNHAGDAANLLI |
| 7 | MAIKATRTYVGSIKNHQQVCDGLDSLGDSASKIWNVARWTADRIWDATGEIPDSGALKSYMKNQPCW |
| | KDLNAQSSQKVIEELSDAFQSWFDLRHKFDEANPPGYRKHGNTRPRSTVTFKEDGFKHDPENDRVRLSK |
| | GSNLKKHFSDFLLCEYQTRPDVDLSEVNSVQNVRAVWNGDEWELHFVCDVELESADTAGDGIAGIDLG |
| | ITNIATVAFPDEYVLYPGNSLKEDKHYFTRAEYDTEGESGPSEQSMWARRKLSKRETHYYHTLTDAIIAE |
| | CVERGVGTLAVSWPEDVRASDXVRLGQDREQEAPLVGVRPHLPVPRIQRRDAGCRGTERERVEHLENV |
| | FTMWRRHEIEPCRTWPVRLFVVRVGSQRRL |
| 8 | MIKKQAFKFLLEPNKGQLSDFLAFAGSCRFVYNKGLALLNENYRAGKKFIGYNKLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPEPIHPSDIKTTIILNNVNSVHLSSGVGGDNTYQAEEKK |
| | KLVRLNKTLTRRKRYSKNWLKTKGKIDRVKSKAARIRLDNIHKATTAICKNHAVVEVVNLMDSVFDKN |
| | DNTLSMRYEFVRQLIYKQEWLGGEIIRRESKLL |
| 9 | MQHLDGFFKLRDQIDYRALPAQANQNVLHMLYRDWKSFFAALADYKAHPDKYEAIPHIPRYADKDGC |
| | KPLIFTNQICKLRKDKHGWYVKFPKAVLQAGCVRDRYDLGKMDLHEQKLKEVRLIPNGDTIKLEIVCEIE |
| | IKEPTITIHEATRVAGIDIGVDNLTAIAFTSGHHPVLIKGNEIKAVNQFYNKQIAHYRSLLRTGKKDSKGIH |
| | QTKRMKRISEKRNRRVKDILHKASRKIIDLCVEEGIEVIVVGNNAGWKKRIHMGKKNNQTFVQIPFHT |
| 10 | MAIQVTRTYVGHITNQQRVRDDLDLLGDAASKLWNVARWTVDRVWDAIGEIPDEGSLKAYMKTRECW |
| | KNLNAFSSQKVIEELSDAFQSWFDVRHKDETANPPGYRKEYDTRPRSTVTFKANGFKHDPDHDQVRLSK |
| | GANLKDGRSDFVLCEYDTRNDVDLGTVDTVQNVRAVWNGDEWEIHFVVKETIETPEPPGDGVAGVDL |
| | GVSNIAAVAFPDKYVLYPGNTIKQDNHYFQQEEYDTEGENGPSKQAQRLRQKRKRRETHFYHTLTKTII |
| | EKCVDRGIGTLVVGWPEDVRSDDLGKTANKWLHTRAFDRLYQYLNYKGKEHDVEVLKENEWNTSKT |
| | CCECGDIADSNRVERGLYVCDSCGLVA |
| 11 | MIKKQAFKFLLEPNKTHMNDFLVFAGSCRFVYNKGLALINENYDSGKKFLNYNQLASELVNWKNEECL |
| | AWLKMAPSQCLQQSLRDLDRAFKNFFSGKSQYPRFKKKGRNDSFRVPCQRVRLDQEKHLVSLPKLGW |
| | VKYRKSREITGVLKNVTISRKLDKWYISFNTEEVVPEPLHPSFSKTKILLNNEWLMQLTACESLVEQFAN |
| | MEGNKKLRNLNNILGRKVKYSSNWLKTKKKIDGVKARSSRRRLDALHKITTAICKKHAIVELVNLTDSL |
| | PDKNNGSVSMTYEFVRQLMYKQEWLGGKVIRLGD |
| 12 | MHRAYKFRLYPNKKQVMLINKTIGCTRFVFNHFLAKRKNVYEQEKKTLNYHECPAMLTQLKKEIEWLK |
| | EVDSTALQSTLKDLDSSYKKFFKEKKGYPKFKSKKNPKQSYTCKMNIKVEGNRIKLPKLGWVEFAKSRE |
| | VEGRIRSATIRRNPSGKYFVSVLCETDIQPHPQIEQTVGVDLGIKDFAILSTGEKVANPKYYRKYEKQLAK |
| | WQRIQSRRQKGGKNRNKARIKVARLHEKIANTRNDFLHKLSTESVKYFV |
| 13 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLARRKKYSKNWLKTKGKIDRVRSKAARIRLDNIHKATTAICKNHAVVEVVNLMDSVSAK |
| | NDKTLSMRYEFVRQLIYKQEWLGGEIIRRESKLL |
| 14 | MADYEAVKVPLDPTPAQERMFRMYAGAARFAYNAALAHMKEQLDERKAQIEAGVAKKDLVKIDNNV |
| | VKFGYWWRANRDTLAPWWPEVASQVFNCAFDNLGHASANFLKSLSGKRQGGPVGFPKFKPRAAAKAF |
| | AFSTITIPDAHGVKLPRIGRVHTLRNVERLVAGRATKTTTIRCEAGRWYASILCETPTPTPPVNTKPEVWV |
| | VFGLDEYIALSDGTRLTNPRPYRHALADLRKASRDLSRKTHGSARYMDQQRKVARIHKRVKALRDNAL |
| | HAASKQLAEHYGVIHVQRIHLARGMRHHVLAQSLADAAFAEFTRQLTYKTARTGASMHMHEPMTVEQ |
| | HVDAMALAQRLATGPLPDA |
| 15 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKSTIVLNNVDSVHLSSVVGGDNTYQAEEK |
| | KKLILLNKTLTRRKKHSKNWLKTKGKIDRLKSKAARVRLDNIHKATTAICKNHAVVEVVNLMDSVSDK |
| | NNNTLSMRYEFVRQLIYKQEWLGGEVICRESKPL |
| 16 | MIKKQAFKFLLEPNKGQLSDFLAFAGSCRFVYNKGLALLNENYRAGKKFIGYNKLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPEPIHPSDIKTTIVLNNVNSVHLSSGVGGDNTYQAEEK |
| | KKLVRLNKTLARRKRYSKNWLKTKGKIDRVKSKAARIRLDNIHKATTAICKNHAVVEVVNLMDSVFDK |
| | NDNTLSMRYEFVRQLIYKQEWLGGEIIRRESKLL |
| 17 | MIKKQAFKFLLEPNKGQLSDFLAFAGSCRFVYNKGLALLNESYRSGKKFIGYNQLASELVQWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTDQTVPDPIHPSDIKTTIVLNNVNSVHLSSGVGGDNTYQAEER |
| | KKLIRLNKTLARRKKYSKNWLKTKGKIDRVRSKAARIRLDNIHKATTAICKNHAVVEVVNLMDSVSAK |
| | NDKTLSMRYEFVRQLIYKQEWLGGEIIRRESKLL |
| 18 | MSDYEAVRIPLDPTPAQERMFRMYAGAARFAYNAALQHMKEQLEQRKAQVDAGVDRKDLVKVDNTV |
| | ITLGYWWRANRDMLAPWWPEIASQVYNCAFDNLGKAAGNFLKSLSGKRQGGLVGFPRFKPRGAAKTF |
| | AYSTVTIPDAHGVKLPRIGRVHTLRNVERLVAGRTVKTTTVRCEAGRWYASILCETPRPSPAVNTSPEV |
| | WCVFGLDDYIALSDGTRIDNPRPYRQALDRLRKVSRDLSRKTHGSGRYMEQQRKVARIHARVKALRNT |
| | MLHEASKRLAEQYGTIHIQQVNLARGMKHHVLAQSLADAAFAEFTRQLEYKTVKTGASVHVHEPMIVE |
| | RHVDGMVLARQLASGPSSDA |
| 19 | MPNEKKNDEEHGVRLSYKFRIYPTPSQCEAIKANIDASRFVYNHYLRARMDAYERTQQEVRRPKPACDE |
| | QGNVQYDQDGKEIWERTEGGKVVFHTVPNPTYDPAAKAMSMEDTSKDLTRLKKELVDEDGKPWLKEA |
| | DATALIYALRNLDTAYQNFFRGIKKGQDVGFPKFKSRKNPVQTYKSGNVKLAGCDLDDGKAEAAVAEI |
| | PSPIPADWDLAGISWNGIVLPKIGKVRARIHRIPEGKFVSCTVERKASGAYYASINVKERELPAYPAATGE |
| | VGITFGASHWAVTSDGQVMDLPERIGRLQRRLAIAQRDLARKEPGSQNYLKQKRKVARVNERIADVRK |
| | AATHNATRELVNGYGTIAARQMNSKDMQQHGSAATKDLPRKVKKMLNRKMIDGNFAEFNRQLAYKS |
| | AWANRSFVEVPGDTPTAQVCSRCGHEELVLARDLRPAWTCSECGAKHDRKANGAQNVLEAGKDILAK |
| | QERSFVTKAKKSREKKRATKPISTAREGASR |
| 20 | MIKKQAFKFRLEPNKSQSSDFFMFAGSCRFVYNKALALLNDNYHSGKKFMGYNQLATELVEWKSEESL |
| | SWLKASPSQCLQQSLRDLDRAFRNFFSGKAQYPKFKKKGRHDSFRIPCQRVRVDQDKKMVSLPKVGWV |
| | KYRKSREIIGELKNVTISMKQDKWYISFNTESMVPDPMHPSDIKTKIVLSDQCEFPIRLDSSMDSSHQLDE |
| | VKKLARLNRILIRRIKYSSNWLKTKGKIDRIKARLARCRLDNIHKVTTAICKKHAVVEVLSLMDSVSDKN |
| | DITLSMRYEFVRQLIYKQEWLGGEVIRRELA |
| 21 | MIKKQAFKFLLEPNKGQLSDFLAFAGSCRFVYNKGLALLNENYRSGKKFIGYNQLASELVEWKNEESLS |
| | WLKEAPSQCLQQSLRDLDKAFRNFFTGKSQYPKFKKKGRHDSFRTPSQRVRVDQEKKLVSLPKVGWVK |
| | YRKSREIIGELKNVTISMKQGKWYISFNTEHTVPDPIHPSDIKTKIVLNNVNSVHLSSGIGGDNTSQAEEK |
| | KKLIRLNKRLARRKKHSKNWLKTKGKIDRVKSKAARLRLDNIHKATTAICKNHAVIEVVNLMGSVSDK |
| | NDNTLSMRYEFVRQLIYKQEWLGGEVIRRESKPL |
| 22 | MIKKQAFKFLLEPNKSQSSDFLAFAGSCRFVYNKGLALLNENYRSGKKFIGYNQLASELVEWKNEESLS |
| | WLKEAPSQCLQQSLRDLDKAFRNFFTGKAQYPNFKKKGRHDSFRIPCQRVRVDQEKKLVSLPKVGWVK |
| | YRKSREIIGDLKNVTISMKHGKWYISFNTEHTVPDPIHPSDIKTTIVLNNENSVHLSTRVGGANTYQAEEK |
| | KKLVRLNKILARRKKHSNNWLKTKGKIDSVISKSARIRLDNIHKATTAICKNHAVVEVVNLMDSVSDKN |
| | DNTLSMRYEFIRQLIYKQEWLGGEIIRR |
| 23 | MIKKKAFKFLLEPSKSQISDVLVFAGACRFVYNKGLALLSENYNNGKPFLNYNKLAPLLVEWKNDNKLE |
| | WLKLCPSQCLQQSLRDLDRAFQNFFSGRSQYPRFKKKGRSDSFRVPCQRVRLDQEKGLVSLPKLGWVK |
| | YRKSRAITGDLKNVTVSRKFDKWYISFNTEEVVSNPVHPSVDKTRILLNDGYVTLCTGGDLSVKKFTSLV |
| | DEKKIKRLNKELSRKVKNSNNWLKNKKKIDKIRLKSGIFRLDAIHKITTTICKKHAVVEVVNVKNFVSDK |
| | NNIATSMRNELVRQLLYKQEWLGGKIIHLDA |
| 24 | MIKQQAFKFALKLNEQQKANMLLFAGACRFVYNKGLALLKESYESGQKHMHYNQLAPLLVEWKSDPA |
| | LSWLKQAPSQSLQQSLRDLDKAFSNFFYGNAEHPRFKKKGQHDAFRFPSQRVKVDQEKQLVLLPKLGW |
| | VKYRKSRDITGDIKNVSISGKLGKWYISFNTQTDIEEPVHPAISKIGVYVDAKKNITLSDGTQYIPPQSLITL |
| | PKQIQRLTNCLRKKNRYSNNWLKSKHRINRLSSRLNQVKVDYLHKASTAISKNHAMIVIADIEKKSFSAD |
| | KQQKNITTCEKSTSIHYELIRQLTYKQEWLGGLVIKLPAEEQKLQRKTRHHEQNVR |
| 25 | MAAKSNPAGPGHGPAAVTMPKTEVLRAYRFALDPSGAERAALSRYAGACRWAYNYALARKTRAHQA |
| | WADRRSAYLEAGLSEAEAKERIRADGAELTDRIKVWDHHRKTLTLTVADKPPLPAMQSPAGQEALVRR |
| | LAAARADAAGTSSERELLAEGRAMVNALKAQAFTAGFRTPTAIDTSALWRMDRDLPQQEGGSPWWRE |
| | VNVYCFTSGFDRAQAAWKNWQDSLAGRRAGQRHGYPRFKKKGHTESFTLFHDVKRPIIRLESYRRLVM |
| | PGLGSIRIHDSGKRLARLVERGQAVIQSVTVTRGGHRWYASVLAKVQQDVPVLWEHVHDDGTRTSYLS |
| | RTQAEKAADNGGHVEQIGRPTARQRAGGLVGVGLGSHYLAALSSPLDPADPATALVQHPRLLADSLAK |
| | LSKAQRAMSRCQQGSGRWSKATAGVCRIHQQITVRRASFLHGLSKKLATGFTHVAIEDLDITALTTSAK |
| | GTRDKPGKNVKAQARFNRHLLDAGLGSLRKKLAYKTAWYGSQLVVLDQGEPVTATCAKCKERNPSSD |
| | PSCSTFHCPSCGAAVHRHENSTANIVDAAHRKLTTVASDRGETQNARRATASPAARKAPGKGQ |
| 26 | MAAKSHPAGRGHGPAAVTMPRAEVLRAYRFALDPSGAERAALSRYAGACRWAYNYALARKMRAHQ |
| | AWADRRSAYLAGGLSEAEAKERIRADGAELTDRIKVWDHHRKTLTLTVAGKPPLPAMQCPAGQEALV |
| | RRLAAARADAAGTGSERELLAEGRAMVNALKAQAFTAGFRTPTAIDTSALWRMDRDLPQQEGGSPWW |
| | REVNVYCFTSGFDRAQAAWKNWQESLAGRRAGRHHGYPRFKKKGHTESFTLFHDVKRPIIRLESYRRL |
| | VMPGLGSIRIHDSGKRLARLVERGQAVIQSVTVTRGGHRWYASVLAKVQQDVPVLWEHVHDDGTRTS |
| | YLSRTQAEEAAGSGGHVEQIGRPTARQRAGGLVGVGLGSHYLAALSSPLDPADPATALVQHPRLLADSL |
| | AKLSKAQRAMSRCQQGSRRWSKATAGVSRIHQQITVRRASFLHGLSKKLATGFTHVAIEDLDITALTTS |
| | AKGTRDEPGKNVKAQARFNRHLLDAGLGSLRKKLAYKTAWYGSQLVVLDQGEPVTATCAKCKERNPS |
| | SDPSCSTFHCPSCGAAVHRHENSTANIVDAAHRKLTTVASDRGETQNARRATASPGARKAPGKGH |
| 27 | MSIYKNFEYRVYPTDEQKKWFEEHFEVNRFLYNHLLSMSIKKYNTEVDERFLRLIKDIDFYSEKIQQWTQ |
| | IDYEKLYKKAKKGVKIYSKNEFSKLITKAVNNPDFPWVNKSYDGRAMREVATSVDTAYKNFFKGKDFP |
| | RFKKKYSVRTLRFPVSKQGEWYSIRFESDKILVLPKKIKLRIVQHRPFEGEVIAATIKKAQSGKWFVTILSR |
| | VDPPTQLIKTGDIIALNRGVREYMIGYDSNHKLINYAPFVKDPTLISKINKLHKKLSQKYKSAKQESRSLR |
| | DSKNYQKNKESLARLYEKLKFQKEYYLQQLSRKIIEDYDLIILESLSIKELASSNIGEKVKSGERIVQRRFS |
| | KKIMGMSHYRLETLLKEKAELYGKRVVMLPKGFNSNGVCSECGTIFEESIPLNNKEFICPNCNIKITRGEN |
| | SVKNILREGMKYL |
| 28 | MQLRYSFRLYPRPGQRAALARAFGCARVVFNDAVRAREDARRQGVPFPKAADLSRTLITQAKQTAERS |
| | WLGEVSAVVLQQSLRDAESAYRNFFASLKGERKGPKLGAPRMKSRKDARQSIRFTTNARWSLTPAGRL |
| | NLPKIGEVRVRWSRTLPAVPSSVTVIKDAAGRYFASFVIDTDPAADAARVPKADQSIGIDLGLTHFAVLS |
| | DGTKIDSPRFLRRAEKKLKKAQRELSRKQKGSKNREKARWKVARAHAKVTDARRDFHHQL |
| 29 | MQLRYSFRLYPQPGQRTALAKAFGCARVVFNDAVRAREDARRQGLPFPKAADLSRTLITQAKQTAERS |
| | WLGDVSAVVLQQSLRDAESAYRNFFASLKGERKGPKLGAPRMKSRKDARQSIRFTTNARWSITPGGRL |
| | NLPKIGEVRVKWSRTLPAVPSSVTVIKDAAGRYFASFVIDTDPAADLEQMPDAETSIGIDLGLTHFAVLSD |
| | GTKIDSPRFLRRAEKKLKKAQRDLSRKQKGSKNREKARWKVARAHAKVT |
| 30 | MQLRYSFRLYPQPGQRTALARAFGCARVVFNDAVRAREDARRQGLPFPKAADLSRTLITQAKHTAERS |
| | WLGEVSAVVLQQSLRDAESAYRNFFASLKGERKGPKLGAPRMKSRKDARQSIRFTTNARWSLTPAGRL |
| | NLPKIGEVRVKWSRTLPAVPSSVTVIKDAAGRYFASFVIDTDPAADAARMPKADQSIGIDLGLTHFAVLS |
| | DGTKIDSPRFLRRAEKKLKKAQRDLSRKQKGSKNREKARLKVARAHAKVT |
| 31 | MEIKRAYKFRFYPTFEQATMLAQTFGCAGFVYNRMLLVRSDAGYTEKKRIGCHATSSLLTKLKKEPEFE |
| | WLNKAPSVPVQQSLRHLQTAFGNFFAKRAKYPSFKRKYGRHSAEYTSSAFKWDGKSLKLEKMKDPLNI |
| | RWSCTLPKAAKLTTAMISKDLTGRYRVSMLCDDSVALKPKVSGKVGIGLGLTHFAILSTGEIVGIERWYP |
| | SSKRCLGCGHTVNKMPLNAREWTCPECGSIHDRDINAARNVLAAGLAVPVLGESISPVCI |
| 32 | MLLFAGACRFVYNKGLALLKESYESGQKHMHYNQLAPLLVEWKSDPALSWLKQAPSQSLQQSLRDLD |
| | KAFSNFFYGNAEHPRFKKKGQHDAFRFPSQRVKVDQEKQLVLLPKLGWVKYRKSRDITGDIKNVSISGK |
| | LGKWYISFNTQTDIEEPVHPAISKIGVYVDAKKNITLSDGTQYIPPQSLITLPKQIQRLTNCLRKKNRYSNN |
| | WLKSKHRINRLSSRLNQVKVDYLHKASTAISKNHAMIVIADIEKKSFSADKQQKNITTCEKSTSIHYELIR |
| | QLTYKQEWLGGLVIKLPAEEQKLQRKTRHHEQNVR |
| 33 | MNQSVSNPTHLRTLRLRVKDKHAAELARQARAVNYVWNYINELSERSIRERGVFLSAFDLHRYTTGAS |
| | KALGLHSHTVQKTSASYVQARIQFRKRKLAWRKSGGVRRSLGWVPFNTGHARWRNGQVHFNGTAYG |
| | VWDSYGLAGFTLRSGSFSEDSRGRWYFNVAVETETKLSAGKSVIGIDLGCKEAATASNAEKLRGRWYR |
| | DDEKALATAQRAGKKRQVKKIHARIKNRRKEDTHQFTTGLVEKSGAIFVGNVSSKAMVKTNMAKSAL |
| | DAGWYSLKKTLEYKCASAGVLYQEVNEAYSTRTCSECGALSGPKGLKELGISGPRRGNGAVLSVEART |
| | TAM |
| 34 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISFNQGKWYISFNTEQTVPDPIHPSEIKTTIALNNVYSVHLSSGVGGDNTYQAEEK |
| | KKLIRLNKTLTRRKKYSKNWLKTKGKIDRVRSKAARIRLDNIHKATTAICKNHAVVEVVNLMDSVSAK |
| | NDNTLSMRYEFVRQLIYKQEWLGGEIIRRESKLL |
| 35 | MIKKQAFKFLLELNKSQSSDFLAFAGSCRFVYNKGLALLNENYRSGKKFIGYNQLASELVEWKNEESLS |
| | WLKEAPSQCLQQSLRDLDKAFRNFFTGKAQYPKFKKKGRHDSFRIPCQRVRVDQEKKLVSLPKVGWVK |
| | YRKSREIIGDLKNITISMKHGKWYISFNTEHTVPDPIHPSDIKTTIVLNNENSVHLSTRVGGANTYQAEEK |
| | KKLVRLNKILARRKKHSNNWLKTKGKIDSVISKSARIRLDNIHKATTAICKNHAVVEVVNLMDSVSDKN |
| | NNTLSMRYEFVRQLIYKQEWLGGEVIRRESKPL |
| 36 | MIKKQAFKFLLEPNKGQLSDFLAFAGSCRFVYNKGLALLNENYRAGKKFIGYNKLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPEPIHPSDIKTTIVLNNVNSVHLSSGVGGDNTYQAEEK |
| | KKLVRLNKTLTRRKRYSKNWLKTKGKIDRVKSKAARIRLDNIHKATTAICKNHAVVEVVNLMDSVEDK |
| | NDNTLSMRYEFVRQLIYKQEWLGGEIIRQESKLL |
| 37 | GFVVLIGFIVIKKKAFKFLLEPSKSQISDVLVFAGACRFVYNKGLALLSENYNNGKPFLNYNKLAPLLVE |
| | WKSDHKFEWLKLCPSQCLQQSLRDLDRAFQNFFSGRALYPRFKKKGRSDSFRVPCQRVRLNQEKGLVS |
| | LPKLGWVKYRKSREVTGNLKNVTISKKLDKWYISFNTEEFVSEPVHPSINKTKVLLNDGYVTLCAGNEV |
| | SVESFTGIVDEKKIKRLNKELSRKVKHSNNWLKSKKKIDRIRTRSGNFRLDALHKITTAICKKHAVVEVV |
| | DVKNFVSDKNNIAKNMRYEFVRQLLYKQEWLGGKIVQLDA |
| 38 | MNDNRRPSAPKRTTQYNTIKIRLYPNQEQEELFQRTFGCCRYIWNRMLADHERFYYETDAHFIPTPAKY |
| | KTEAPFLKEVDHQALTQEYNKLSQAFRNFFRNPASFGYPKFKRKKDDRDSFSACNQVMGNSATIYITQD |
| | AVRMTKAGLVRAKFPRRPRSGWKLTRITVERTKTGKYYGYLLFACPVHAPEPVKPTADTTIGLKYSLTH |
| | FYVRDDGITADPPRWLRQSQDKVSSIQEKLNRMQPGSRNYREMVQKYRLLHEHIANQRRDFLHKESRRI |
| | ANDWDAVCIRDDSLKAISEELGGSDIHDTGFGMFREMLRYKLDRQGKQLLEVGRFDPTTKVCSVCGAIN |
| | ETLSPKARHWVCPVCGAEHKRGKNAAVNIKAHGLACYQNKQVAEAVS |
| 39 | MAQTKTWNTTIKVRLDPTPAQAAFFDENFNCCRYLWNQMLSDQIRFYTETDAHFIPTPAKYKKDAPFLK |
| | EADSNALVSVHQNLHKAFQRFFSNPSRYRHPTFKSKKRCKNSYTTYCQYYRSGKGTSIYLTKDGIRLPK |
| | AGLVKARLHRRPLHWWTLKTATISKTSSGKYYCSLVFAYTTKPSRQIPPTPETTLGLNYSLSHFYIDSNG |
| | HAADPPHWLARSQDKLRYMQQQLARMQPGSRNYEQQLYKIQRLHEHISNQRKDFLHKESRRIANAWD |
| | AVCVKDTNLVKMSQAIKLGHVMDAGYGRFRSYLQYKLERLGKPYIVVEKYFPSTKTCHHCGSVNEALP |
| | AGAKRWTCPICGTTLDRAKNAAQNLRDQGLVQYSASQRQRASA |
| 40 | MADYEAVKVPLDPTPAQERMFRMYAGAARFAYNAALAHMKEQLDERKAQIEAGVAKKDLVKIDNNV |
| | VKFGYWWRANRDTLAPWWPEVASQVFNCAFDNLGHASANFLKSLSGKRQGGPVGFPKFKPRAAAKAF |
| | AFSTITIPDAHGVKLPRIGRVHTLRNVERLVAGRATKTTTIRCEAGRWYASILCETPTPTPPVNTKPEVWV |
| | VFGLDEYIALSDGTRLTNPRPYRHALADLRKASRDLSRKTPGSARYMDQQRKVARIHKRVKALRDNAL |
| | HAASKRLAEHYGVIHVQRIHLARGMRHHVLAQPLADAAFAEFTRQLAYKTARTGASVHMHEPMTVEQ |
| | HVDAMALAQRLANGPSPDA |
| 41 | MAKREKKDDVVLRGTKMRIYPTDRQVTLMDMWRRRCISLWNLLLNLETAAYGAKNTRSKLGWRSIW |
| | ARVVEENHAKALIVYQHGKCKKDGSFVLKRDGTVKHPPRERFPGDRKILLGLFDALRHTLDKGAKCKC |
| | NVNQPYALTRAWLDETGHGARTADIIAWLKDFKGECDCTAISTAAKYCPAPPTAELLTKIKRAAPADDL |
| | PVDQAILLDLFGALRGGLKQKECDHTHARTVAYFEKHELAGRAEDILAWLIAHGGTCDCKIVEEAANHC |
| | PGPRLFIWEHELAMIMARLKAEPRTEWIGDLPSHAAQTVVKDLVKALQTMLKERAKAAAGDESARKTG |
| | FPKFKKQAYAAGSVYFPNTTMFFDVAAGRVQLPNGCGSMRCEIPRQLVAELLERNLKPGLVIGAQLGLL |
| | GGRIWRQGDRWYLSCQWERPQPTLLPKTGRTAGVKIAASIVFTTYDNRGQTKEYPMPPADKKLTAVHL |
| | VAGKQNSRALEAQKEKEKKLKARKERLRLGKLEKGHDPNALKPLKRPRVRRSKLFYKSAARLAACEAI |
| | ERDRRDGFLHRVTNEIVHKFDAVSVQKMSVAPMMRRQKQKEKQIESKKNEAKKEDNGAAKKPRNLKP |
| | VRKLLRHVAMARGRQFLEYKYNDLRGPGSVLIADRLEPEVQECSRCGTKNPQMKDGRRLLRCIGVLPD |
| | GTDCDAVLPRNRNAARNAEKRLRKHREAHNA |
| 42 | MNRGYKYRIYPNKEQEILIQKTFGCARFIYNKMLENRITTYEKYKENKTELKKQKYRTPASYKGEFPWL |
| | KEVDSLALANVQMDLDKAYKNFFRDSKVGYPKYKSKHKDRKSYTTNNQKGSIRIIDENHIRIPILKDLKI |
| | KMHRPLKENSSIKAATISQTPTGKYFISILVEYPEDKITPIKAMQERVLGLDYSSTSLYIDDKGLESEYPKY |
| | YRQAEMKLKKEQRKLSKKKEDSKNREKQRQKVAKLHEKVANQRKDFLHKKSRQIANVYDAV |
| 43 | MLRAAKFRIYPTAAQEAFLWAQWGAVRKCWNMALFLKKHYYRTRGVSLDLIHEIKPLIARAKKSKKYT |
| | WLKEYDSMALQESVRNLNKGYRAFFEGRAGYPHYKSRRGPQSSYHCTNVSVGPNYVRVPKMEPIKARI |
| | HREVVGKVKSITLEADAAGDYYAAVLWEDGLAEKDPLKEIYEDQVIGIDVGIKDLLTESNGRKEPNPKH |
| | LKRARKVLRRRCRQFSRTQKGSRRREKARRRLARAHKRVANARTDNLHKVSSRLVNDLWTTHVCQAP |
| | STVTRELRSEAAGSELVERLIAAAGGIAGLVPSGTEPIPVKRSWNSEHGLQAERSICRVSRFTGPPPIQCRY |
| | VRMRQAKRSPHSDLATLHRLIRVMSFWLLPAVFQSQ |
| 44 | MKQKKQDGHAEPGRVVQYNTIKVRLYPTPEQEELFQKTFGCCRYIWNQMLSDHERFYLETDAHFLPTP |
| | AKYKKGAPFLKEVDNQALTQEYNKLSQAFRNFFRNPAAFGYPKFKRKKDDRDTFSACNHVMGNSATIY |
| | TTRDAVRMTKAGLIRAKFPRRPRSGWRLVRVTVERTKTGKYYGYLLYACPARQPEPVAPVEERTVGLK |
| | YSLSHFYVADDGTAADPPRWLRQSQDKLVAVQRKLSRSQPGSQNYQELVQKYRLLHEHITNQRRDFLH |
| | KESRRIANAWDAVCIREDSLKAISETLGGSAVRDTGFGMFRELLRYKLERQGKQLLEVDRLFPTTKVCS |
| | ACGAVNETLAPRARRWVCPVCGAEHRRGVNAAVNIKARGLVRHQHQQTAAAAS |
| 45 | MIKQQAFKFALKPNKQQKNDMLLFAGACRFVYNKSLSLLKDNYQSGGKHMHYNQLAPKLVEWKSESD |
| | LSWLKEAPSQSLQQSLRDLDKAFSNFFGGKAEHPRFKKKGQHDAFRFPSQRVKVDQEKQLVLLPKLGW |
| | VKYRKSRDITGDIKNVTISGKLGKWYISFNTQTDIVEPEHPTTSNVGLYIDNNRQITLSDGTQYFPPEDLRT |
| | LPKKIQKFKLRLRKKTHHSNNWLKSKRKINLLRSRLSQIKNDYLHKTSTAISKNHAMIVIADVEKKSFSG |
| | DKQQKNVESYETLTSVQYELIRQLTYKQEWCGGIVIKLPDNTSISSIDNHASKNEYIAEKLNLNADSLRTK |
| | ACNLLAAGLAVTACGGDVVKRSPVKQEP |
| 46 | MLDPNQEQLSMMTVISGACRYVENKALEIAVQNHIAGEKYVPYNKTAPLLVQWKSQESLSWLKLAPSQ |
| | SLQQSLKDLDRAFHGYISRKSGFPKFRKKGTDESFRFPQQRVKVDEVNKKVYLPKIGWVRYRKSRDVIG |
| | EIKNITISQTANKWYVSFQTQIEIPDPVHTSSLTAKVTLSDEGTILLSDGKKYALPETYSRHFNQLNKLIRQ |
| | KNRKIKSSQSWLAMHHSIILKKAKLRNILMDFLHKTSTLICNNHAKISVDTEKGNSARKTSPLPVNFKPYE |
| | FLRQLKYKQSWNGGSVCVEQT |
| 47 | MITTYHYRIKDSGKSGRALKKMSSSVNFVWNYCKNTQKEALKNRTVKKIIDPLSGKTIFVPYFFTKFEM |
| | NSLVSGSSKELGIHSQTIQAISEEYTTRRKQFKNILRWRGKNSLGWIPFKATAIKINQDKVSYHKNTFRFW |
| | NTREIPDDAIIKSGSFAQDSRGRWYLNITFETKTSQYSNENLIENGVFIDSNHLAKCSNGIKFDRPKISIKYV |
| | RKIKISNKIKKNILMKKSKLKLIKRKAPKIKQEKNLRAKLENIKLDHFHKQSTKIINFSSAIITNQITAKRKK |
| | SYKNNHFISFGAISKPFQNMLCYKAIRAGRTFKVIPEKDLIWAFSKCCSSQPRTNLRIRVWKCRECGKINH |
| | FSTKADKNLLSVYKNPLRIGHDTPRSI |
| 48 | MKQKKQDGHAEPSRVVQYNTIKVRLYPTPEQEELFQKTFGCCRYIWNQMLSDHERFYLETDAHFIPTPA |
| | KYKKGAPFLKEVDNQALTQEYNKLSQAFRNFFRNPSAFGYPKFKRKKDDRDTFSACNHVMRNSVTIYT |
| | TRDAVRMTKAGLIRAKFPRRPRSGWRLVRVTVERTKTGKYYGYLLYACPMRQPEPVAPVEERTVGLKY |
| | SIAHFYVTDDGTSADPPRWLRQSQDKLSAVQRKLSRSQPGSQNYQELVQKYRLLHEHIANQRRDFLHKE |
| | SRRIANAWDAVCIREDSLKAISEKLGGSAVRDTGFGMFRELLRYKLERQGKQLLEVDRLVPTTKVCSAC |
| | GAVNETLAPRARRWVCPVCGAEHRRGVNAAVNIKARGLIQHQQTAEAVS |
| 49 | MIKKKAFKFLLEPSKSQISDVLVFAGACRFVYNKGLALLSENYNNGKPFLNYNKLAPLLVEWKSDHKFE |
| | WLKLCPSQCLQQSLRDLDRAFQNFFSGRALYPRFKKKGRSDSFRVPCQRVRLNQEKGLVSLPKLGWVK |
| | YRKSREVTGNLKNVTISKKLDKWYISFNTEEFVSEPVHPSINKTKVLLNDGYVTLCAGNEVSVESFTGIV |
| | DEKKIKRLNKELSRKVKHSNNWLKSKKKIDRIRTRSGNFRLDALHKITTAICKKHAVVEVVDVKNFVSD |
| | KNNIAKNMRYEFVRQLLYKQEWLGGKIVQLDA |
| 50 | MITTYRYRIKDSGSTKKKLLKMANGVNFIWNFCKETQSNALKNKPVKVITDPKTKKIYYTPYFFTQYEM |
| | NELVAGSSKELGLHSQTVQAVAEEYITRRKQFKKLLRWRGRNSLGWIPFKSSGIKIVKDVVQYNKLKFR |
| | FWNSRNLPSDAHIKSGSFAQDNCGRWYINITIETKNNLYNKNSTSESAIFLSNYKGIIYQNESDSVKPNFSS |
| | KLIAKIKKLNIAKKKRVIQRKKDKLKEKPKPIGRKEKKILNKVANIKQDLFHKESTKIINNNRLVITNEIIA |
| | AKKRIQSRNSFISTRLNVKHFQNMLCYKALRAGKVVSIVSNKNLSLVPFQCCSLQSQFILRKRTFVCKICH |
| | KRTSFMTSARNNLLLAAKHLLRIGHDTP |
| 51 | MQLRYNFRLYPTPGRRQALARAFGCARAVFNDALRMRRDAHAGGLPYLSDGELSKRVITVAKKTPERA |
| | WLAEVSAVVLQQALADLSTAYRNFFNSVSGKRKGPKVAPPRFRSRKDSRQSIRFTRNARFQITAGGGLH |
| | LPKIGAMRVRWSRDLPSEPSSVTVIKDASGRYFASFVVETGEEPLPETGGEVGIDLGLTHFAVLSNGRKID |
| | NPRFLRRYERRLKKAQRALSRKEKGSANRSKAVARAARAHARVADARRDHHHRLSTAIIRDN |
| 52 | MPNEKKGDEEHGVRLSYKFCIYPTPSQCEAIKANIDASRFVYNHYLRARMDAYERTQQEVRRPKPACDE |
| | QGNVQYDQDGKEIWERTEGGKVVFHTIPNPTYDPAAKVMSMFDTSKDLTRLKKELVDEDGKPWLKEA |
| | DATALIYALRNLDTAYQNFFRGIKKGQDVGFPKFKSRKNPVQTYKSGNVKLAGCDLDDGKAEAAVAEI |
| | PSPIPADWDLAGISWNGIVLPKIGKVRARIHRIPEGKFVSCTVERKASGAYYASTNVKERELPAYPAATGE |
| | VGITFGASHWAVTSDGQVMDLPERIERLQRRLAIAQRDLARKEPGSQNYLKQKRKVARINERIADVRKA |
| | ATHNATRELINGYGTIAARQMGSKEMQQHDGAATKDLPRKVKKMLNRKMIDGNFAEFNRQLAYKSA |
| | WANRTFVEVPGDTPTAQVCSRCGHEELVLARDLRPAWTCPECGAKHDRKANGAQNVLEAGKDILAKQ |
| | EQSFVTKAKRSREKKRAAKPKNSEKQNDWQL |
| 53 | MADYEAVKVPLDPTPAQERMFRMYAGAARFAYNAALAHMKEQLDERKAQIEAGVAKKDLVKIDNNV |
| | VKFGYWWRANRDTLAPWWPEVSSQVYNCAFDNLGKASSNFLKSLSGKRKGGPVGFPKFKPRGATKAF |
| | AFSTITIPDAHGVKFPRIGRVHTLRNVERLVAGRATKTTTIRCEAGRWYASILCENPSATPPVNTKPEVWV |
| | VFGLDEYIALSDGTRIDKTAPYRQALDRLRKASRDLSRKTHGSGRYMEQQRKVARIHARVKALRNTML |
| | HEASKRLAERYGIIHIQQINIARGMKHHVLAQSLMDAAFAEFTRQLEYKAAGTGASVHVHEPMTVERHV |
| | DGMVLARHLADDSSSDA |
| 54 | MLEPSKSQISDVLVFAGACRFVYNKGLALLSENYNNGKPFLNYNKLAPLLVEWKSDHKFEWLKLCPSQ |
| | CLQQSLRDLDRAFQNFFSGRALYPRFKKKGRSDSFRVPCQRVRLNQEKGLVSLPKLGWVKYRKSREVT |
| | GNLKNVTISKKLDKWYISFNTEEFVSEPVHPSINKTKVLLNDGYVTLCAGNEVSVESFTGIVDEKKIKRL |
| | NKELSRKVKHSNNWLKSKKKIDRIRTRSGNFRLDALHKITTAICKKHAVVEVVDVKNFVSDKNNIAKN |
| | MRYEFVRQLLYKQEWLGGKIVQLDA |
| 55 | MKQEKQDGHAEGNRVIQYNTIKVRLCPTPEQEELFQKTFGCCRYIWNQMLSDHERFYEETDAHFIPTPA |
| | KYKKGAPFLKEVDNQALTQEYNRLSQAFRNFFRDPKTFGYPKFKRKKDDRDSFTACNQFFGSSATIYAT |
| | RDAVRMTKAGLVKAKFSRRPRSGWKLTRLTVERTKTGKYYGYLLYTCPTYQPEPVEATAERTIGLKYS |
| | VSHFYVADNGNSADPPRWLRQSQEKLAVVQRKLSRSQPGSQNYQELVQKYRLLHEHIANQRRDFLHKE |
| | SRRIANAWDAVCIREDSLRAISGKLGGSAVHDTGFGMFRELLRYKLERQGKQLLEVDRLVPTTKVCSAC |
| | GAVNETLSIRARRWVCPVCGAEHRRGMNAAINIKASGLVKGQSQQAAAALPLL |
| 56 | VIKKKAFKFLLEPSKSQISDVLVFAGACRFVYNKGLALLSENYNNGKPFLNYNKLAPLLVEWKSDHKFE |
| | WLKLCPSQCLQQSLRDLDRAFQNFFSGRALYPRFKKKGRSDSFRVPCQRVRLNQEKGLVSLPKLGWVK |
| | YRKSREVTGNLKNVTISKKLDKWYISFNTEEFVSEPVHPSINKTKVLLNDGYVTLCAGNEVSVESFTGIV |
| | DEKKIKRLNKELSRKVKHSNNWLKSKKKIDRIRTRSGNFRLDALHKITTAICKKHAVVEVVDVKNFVSD |
| | KNNIAKNMRYEFVRQLLYKQEWLGGKIVQLDA |
| 57 | VIKKQAFKFLLEPNKGQLSDFLAFAGSCRFVYNKGLALLNENYRAGKKFIGYNKLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPEPIHPSDIKTTIILNNVNSVHLSSGVGGDNTYQAEEKK |
| | KLVRLNKTLTRRKRYSKNWLKTKGKIDRVKSKAARIRLDNIHKATTAICKNHAVVEVVNLMDSVFDKN |
| | DNTLSMRYEFVRQLIYKQEWLGGEIIRRESKLL |
| 58 | MGSLVIKKQAFKFLLEPNKNHINEFLVFAGSCRFVYNKGLALINENYDSGKKFLNYNQLASELVNWKNE |
| | ECLAWLKMAPSQCLQQSLRDLDKAFKNFFSGKSQYPRFKKKGRNDSFRVPCQRVRLDQEKNLVSLPKL |
| | GWVKYRKSREITGVLKNVTISRKLDKWYISFNTEAVVPEPVHPSFSKTKILLNNECIMQLTSNESLVEQFT |
| | SMEGNKKLRNLNNILGRKVKYSSNWLKTKKKIDSVKARSSRRRLDALHKITTAICKKHAIVELVNLTDS |
| | LPDKSNGFVSMGYEFVRQLMYKQEWLGGQVIRLGD |
| 59 | MIKQQAFKFALKLNEQQKANMLLFAGACRFVYNKGLALLKESYESGQKHMHYNQLAPLLVEWKSDPA |
| | LSWLKQAPSQSLQQSLRDLDKAFSNFFYGNAEHPRFKKKGQHDAFRFPSQRVKVDQEKQLVLLPKLGW |
| | VKYRKSRDITGDIKNVSISGKLGKWYISFNTQTDIEEPVHPAISKIGVYVDAKKNITLSDGTQYIPPQSLITL |
| | PKQIRRLTNCLRKKNRYSNNWLKSKHRINRLSSRLNQVKVDYLHKASTAISKNHAMIVIADIEKKSFSAD |
| | KQQKNITTCEKSTSIHYELIRQLTYKQEWLGGLVIKLPAEEQKLQRKTRHHEQNVR |
| 60 | MLEPSKSQISDFVVFAGACRFVYNKGLALLSENYNNGKPFLNYNKLAPLLVEWKSDNKLEWLKLCPSQ |
| | CLQQSLRDLDRAFQNFFSGRSQYPRFKKKGRSDSFRVPYQRIRLNQDKGLVSLPKLGWVKYRKSREVTG |
| | DLKNVTVSKKFDKWYISFNTEEIVSDPVHPSVNKTKILLNDGYVTMCTGSELSVKKFTSQIDEKKIKRLN |
| | KELSRKVKHSNNWLKSKKKIDRLRSKSGNFRLDALHKITTTICKKHAVVEVINVKNFVSDKNNIATSMR |
| | YEFVRQLLYKQEWLGGEIIQLNA |
| 61 | MLEPNKGQLSDFLAFAGSCRFVYNKGLALLNESYRSGKKFIGYNQLASELVQWKNEESLSWLKEAPSQ |
| | CLQQSLRDLDRAFRNFFTGKSQYPKFKKKGRHDSFRIPCQRVRVDQEKKLVSLPKVGWVKYRKSREIIG |
| | DLKNATISLNQGKWYISFNTDQTVPDPIHPSDIKTTIVLNNVNSVHLSSGVGGDNTYQAEERKKLIRLNK |
| | TLARRKKYSKNWLKTKGKIDRVRSKAARIRLDNIHKATTAICKNHAVVEVVNLMDSVSAKNDKTLSIR |
| | YEFVRQLIYKQEWLGGEIIRRESKLL |
| 62 | MIKQQAFKFALKLNDQQKANMLLFAGACRFVYNKGLALLKESYESGQKHMHYNQLAPLLVEWKSDPA |
| | LSWLKQAPSQSLQQSLRDLDKAFSNFFYGKAEHPRFKKKGQHDAFRFPSQRVKVDQEKQLVLLPKLGW |
| | VKYRKSRNITGAIKNVSISGKLGNWYISFNTQTDIAEPIHPAISKIGVYVGTKKNITLSDGTQYIPPQSLITL |
| | PKQIQRLTNCLRKKNRYSNNWLKSKHRINRLSSRLNQVKVDYLHKASTAISKNHAMIVIADFEKKSFSA |
| | DKQQKNLTTCEKSTSIHYELIRQLTYKQEWHGGLVIKLSAEKNVDAESAWTKACNLLAAGLAVTACGG |
| | EVSKDSPMKQEP |
| 63 | MLRATKVRIYPTSEQAEFLDRQFDAVRFVWNKALAIKVHYYKVRGQSLSPKKHLKPLLAKAKKSRKYS |
| | WLKNADSIALQQVTINLDTAFQNFFNPKLQARFPRFKKKHGKQSSYHCTSVSVGDNWIKIPKCKPIRAKV |
| | HREIVGKVKSITLRRTLTGKYFASILADDTQEQPKQIDNLEANQVVGVDMGITDLAITSTGHKTGNPRFL |
| | KKAQRNLKRKQQALSRCKKGSKGRHKARLLVAKAHERVAFARNDFQHKGRSIQCLTGLLAVGY |
| 64 | MSIYKNFEYRVYPTDEHKKWFEEHFEVNRFLYNHLLSMSIKKYNTEVDERFLRLIKDIDFYSEKIQQWTQ |
| | IDYEKLYKKAKKGVKIYSKNEFSKLITKAVNNPDFPWVNKSYDGRAMREVATSVDTAYKNFFKGKGFP |
| | RFKKKYSVRTLRFPVSKQGEWYSIRFESDKILVLPKKIKLRIVQHRPFEGEVIAATIKKAQSGKWFVTILSR |
| | VDPPTQLIKTGDIIALNRGVREYMIGYDSNHKLINYAPFVKDPTLISKINKLHKKLSQKYKSAKQESRSLR |
| | DSKNYQKNKESLARLYEKLKFQKEYYLQQLSRKIIEDYDLIILESLSIKELASSNIGEKVKSGERIVQRRFS |
| | KKIMGMSHYRLETLLKEKAELYGKRVVMLPKGFNSNGVCSECGTIFEESIPLNNKEFICPNCNIKITRGEN |
| | SVKNILREGMKYL |
| 65 | MSIYKNFEYRVYPTDEQKKWFEEHFEVNRFLYNHLLSMSIKKYNTEVDERFLRLIKDIDFYSEKIQQWTQ |
| | IDYEKLYKKAKKGVKIYSKNEFSKLITKAVNNPDFPWVNKSYDGRAMREVATSVDTAYKNFFKGKDFP |
| | RFKKKYSVRTLRFPVSKQGEWYSIRFESDKILVLPKKIKLRIVQHRPFEGEVIAATIKKAQSGKWFVTILSR |
| | VDPPTQLIKTGDIIVLNRGVREYMIGYDSNHKLINYAPFVKDPTLISKINKLHKKLSQKYKSAKQESRSLR |
| | DSKNYQKNKESLARLYEKLKFQKEYYLQQLSRKIIEDYDLIILESLSIKELASSNIGEKVKSGERIVQRRFS |
| | KKIMGMSHYRLETLLKEKAELYGKRVVMLPKGFNSNGVCSECGTIFEESIPLNNKEFICPNCNIKITRGEN |
| | SVKNILREGMKYL |
| 66 | MAIEVTRTYVGSIQNNRQVCDGLDSLGDSASKIWNVARWTVDRIWNQTGEIPDEGSIKSYMKNQSCWK |
| | DLNAQSSQKVIEELSDAFQSWFDLRHKDDKANPPSYRKHGDERPRSTVTFKEDGFKHDPENNRVRLSKG |
| | SNLKEHFSDFLLCEYRIRPDVDLSEVNKVQNVRAVWSGDEWELHLVCKVSLETNDSAGDEVAGIDLGIK |
| | NIATVAFPDEYVLYPGNSLKQDKHYFKRSEYDTEGENGPSEKSI |
| 67 | KAYKFRMYPNKKQQELINKTFGCCRFVYNKYLAKRIEVYKNDKETFTYKQCSSDLTNLKKELKWLKEP |
| | DKFSLQNALKDLDNAYKKFFKEKAGFPKFKSKKINRFSYKTNFTNGNIMYCGQHIKLPKLGMVKIRDKQ |
| | VPKGRILNATISKEPSGRYYVSLCCTDVDIEVFENTNNQIGLDLGIKEFCISSCGEFIENPKYLKKSLNKLA |
| | KLQRELSRKTIGSLNRNKARLKVARLQEHIANQRNDFLQKLSTK |
| 68 | MKQKRAFKYRVYPTPEQQQILAQTFGCCRFVYNWALRKKTDAYYNDHQRLYYKELSLLLTDLKKQEE |
| | THWLNEVSSVPLQQALRHLDKAFLNFFEGRAKYPTFHKKRNTQSATYTANAFTWRNGSLTLAKMSEPL |
| | QIVWSRPLPNEAIPSSVTITKDCADRYFISLLVEEEIAHLPCNEKAIGADLGLKSFVVLSTGEVVGNPRFFH |
| | KDEKKLAKAQRRHAKKKKGSKNRDKARLKVARIHARIADRRRDAPAQALYPPDS |
| 69 | MEPTREQGEALERMAGARRWVWNWGLARRKEAYAATGKGLTYNQQAALLTALKQQPETAWLKEAD |
| | SQLLQQALKDLDRAFKAFFEKRAGFPQFKSKKRDTPRFRIPQRVKVEGSKVYIPKVGRVKIRQSQPIDCAI |
| | KGATFKRDTQGHWYVTLTAEFEMPEVPLPPANPERVVGIDLGLKDFAVLSDGTRIAPPKFYRKGLSKLR |
| | RAQRELSRKQKGGKNRDKARHRLSKVHARVRNQRQDWLHKLTTGLVQKYDGAVHRRPEPEGDGENQ |
| | AVHIGAGRGVGRVPQATGVQNGLAPQTSRRD |
| 70 | MEPTQAQSDALLRMAGARRFVWNWGLARRKEAYAATGKGLTYNQQAAELTTLKQQPETVWLKEADS |
| | QLLQQALKDLDRAFKAFFERRAGFPQFKSRKRDEPRFRIPQRVKVENSKVYVPKVGWVRIRQSQPIDCPI |
| | KGATFKREADGHWYVTLTAEFEMPDVPLPPANPERVVGVDLGLKDFAVLSDGTRIAPPRFYRKGLAKL |
| | RRAQRELSRKRRGSKNREKARHRLSKVHARVRNQRQDWLHKLTTGLVQKYD |
| 71 | MQLNKTAKRILSIRISGKQRKEKISNLLYSLAQFRNLLIIFNKIYQQNYGRWILNESYLYALVNNKGYKPR |
| | ESKENFIEKLKEFKTITDNIEKVNQLKDFQDKLIKQKQKIKNNYTVQTLIRQLIKDYKSFFKSIQKYKENSN |
| | SFNAIPRPPKAKKLKDIPSFTAELNVNTFKVLEEEKGKHLLITLTNNKEEKQYLKVKLPKDFNYEIKSARI |
| | KFIASDIYVDIVYTIPETQINSNQEKTHIAGIDLGLDNLITLFSTNKELQTIIVSGKEIKSINQWYNKEKAKL |
| | QSKIDNIQNQINKLQKDNLDTTALEKEKKLLIKKQKELSAYRNRWITDTFHKITRKITDFLNETGHKEVYI |
| | GKGATESKNGINLSTKTNQNFVNIPFRKLINQLKYKLEEYGVKLTEVAEEFTSKTSPFADLHKVLETGKE |
| | YLKAKTEGNEGILKQLKEKLNQLYNGIRIKRGLYKDNITNKVFNANAVGSYNILRKEAKPLIDEETLIDK |
| | LSRPIRLTLNLISKVTCESLLEIAGRRPLRVHCKRTLVNNFL |
| 72 | MIKKKAFKFLLEPSKSQISDVLVFAGACRFVYNKGLALLSENYNNGKPFLNYNKLAPLLVEWKNDNKLE |
| | WLKFCPSQCLQQSLRDLDRAFQNFFSGRSQYPRFKKKGRSDSFRVPCQRVRLDQEKGLVSLPKLGWVK |
| | YRKSRAITGDLKNVTVSRKFDKWYISFNTEEVVSNPVHPSVDKTRILLNDGYVTLCTGGDLSVKKFTSLV |
| | DEKKIKRLNKELSRKVKNSNNWLKNKKKIDKIRLKSGSFRLDAIHKITTTICKKHAVVEVVNVKNFVSD |
| | KNNIATSMRYELVRQLLYKQEWLGGKIIHLDA |
| 73 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISFNQGKWYISFNTEQTVPDPIHPSEIKTTIVLNNVNSVHLSSGVGGDNTYQAEEK |
| | KKLIRLNKTLTRRKKYSKNWLKTKGKIDRVRSKAARIRLDNIHKATTAICKNHAVVEVVNLMDSVSAK |
| | NDNTLSMRYEFVRQLIYKQEWLGGEIIRRESKLL |
| 74 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLTRRKKYSKNWLKTKGKIDRVRSKAARIRLDNIHKATTAICKNHAVVEVVNLMDSVSAK |
| | NDNTLSMRYEFVRQLIYKQEWLGGEIIRRESKLL |
| 75 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLARRKKYSKNWLKTKGKIDRVRSKASRIRLDNIHKATTAICKNHAVVEVVNLMDSVSAK |
| | NDNTPSTRYEFVRQLIYKQEWLGGEIIRRESKLL |
| 76 | MEVKKAYKFRIYPNQTQTQLFEQTFGCSRFLYNRALYETKTAGTKFRKTPAIKEIGKLKKAFTWLKAVD |
| | SIALQAAIENLDDAFIRFYRKQTKFPRFKSKKNLVKSYTTKAVNGNIQLEDNKIKLPKVGWIRYAKSREV |
| | KGTIKRVTVRKNAAGKYFVSILAVVEHNYNRNNTNETVGLDLGLTDFLITNEGSKIKNPRHLKKYEQKL |
| | QHAQRTMSRRTIGSSNWHKQKIKWFASTKRSLMPAGIFSINYLAN |
| 77 | MEKAYKFRMYPNKKQQELINKTFGCCRFVYNKYLAKRIDVYKNNKETFTYKQCSSDLTNLKKELKWL |
| | KEPDKFSLQNALKDLDNAYKKFFKEKTGFPKFKSKKINRFSYKTNFTNGNIMYCGQHIKLPKLGMVKIR |
| | DKQVPQGRILNATISKEPSGRYYVSLCCTDVDIEAFENTNNHIGLDLGIKEFCISSCGEFIENPKYLKKSLN |
| | KLAKLQRELSRKTIGSLNRNKARLKVARLQEHIANQRKDFLQKLSTKLIKENDXKLSILQEMTTFY |
| 78 | MEKAYKFRMYPNKKQQELINKTFGCCRFVYNKYLAKRIEVYKNNKETFTYKQCSSDLTNLKKELKWL |
| | KEPDKFSLQNALKDLDNAYKKFFKEKVGFPKFKSKKINRFSYKTNFTNGNIMYCGQHIKLPKLGMIKIRD |
| | KQVPQGRILNATISKEPSGRYYVSLCCTDVDIEAFENTNNQIGLDLGIKEFCISSYGEFIENPKYLKKSLNK |
| | LAKLQRELSRKTIGSLNRNKARLKVARLQEHIANQRNDFLQKLSTKLIKETILFAYKIYK |
| 79 | MIKKQAFKFLLEPNKGQLSDFLAFAGSCRFVYNKGLALLNENYRSGKKFIGYIQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYVSFNTEQTVPDPIHPSDIKSTIVLNNVDSVHLSSGGGGDNTYQAEEK |
| | KKLIRLNKTLTRRKKHSQNWLKTKGKIDRVKSKAARIRLDNIHKATTAICKNHAVVEVVNLMDSVSDK |
| | NDNTLSMRYEFVRQLIYKQEWLGGEVIRRESKPL |
| 80 | MLKATKIRIYPTTEQAAFLNYQFGAVRFVYNTGLRIISHRYQHHGQSLSAKHDIKKLLPVAKKSRKYSW |
| | LKDADSMALQQACLNLDHAFQCFFDPQQKAGYPSFKSKRGKQSSYHCVGVKAGDDWIKVPKLGPIRAR |
| | VHRKVEGTLKSITLTRTVTGKHYASLLFETEQAAPAPLKDVDAAKIVGLDMGLSHLAIDSNGRKIENPRF |
| | LKRAQQNLKRKQKALSRCQKGSANRAKARLLVAKA |
| 81 | MSLSNGFCTRTSLNVIKNMLKNHKLAKAISEVSWSQFRTMLEYKAKWYGKQIIVVSKTFASSQLCSCCG |
| | YQNKDVKNLKLRKWDCPSCRTHHDRDINASINLKNEANTSTDIVPMGQVHGWYSLRWQIEILFKTWKS |
| | FFQIHHCKKIKPERLECHLYGQLIAILLCSSIMFQMRQLLLMKKKRELSEYKAIYMIKDYFLLLFQSIQKNT |
| | QELSKVLLRLFNLLQQNGRKSHRYEKKTVFDILGVVYNCTLSDNQAA |
| 82 | MNVIRQKHHSTPIHIIDHEHRFVSMQHLDGFFKLRDQIDYRALPAQANQNVLHMLYRDWKSFFAALAD |
| | YKAHPDKYEAIPHIPRYADKDGCKPLIFTNQICKLRKDKHGWYVKFPKAVLQAGCVRDRYDLGKMDLH |
| | EQKLKEVRLIPNGDTIKLEIVCEIEIKEPTITIHEATRVAGIDIGVDNLTAIAFTSGHHPVLIKGNEIKAVNQF |
| | YNKQIAHYRSLLRTGKKDSKGIHQTKRMKRISEKRNRRVKDILHKASRKIIDLCVEEGIEVIVVGNNAGW |
| | KKRIHMGKKNNQTFVQIPFHT |
| 83 | MKTVEFKLNLNQTQQAKVDGWLSVLRWVWNRGLHLLEEFDNNTRWDKSSKSWVPCCPLPWQYYKD |
| | DDGRLIPFTRLAQTKPYRMSCPIPQTYRQPEIESPNHFGILYYFAQKNHLDKPWFCAVPSKSVSGTLKALT |
| | DAWWEYKSGKRSSPRYKRYKDKIKSLVNNNSKSIKISGRQITLPKLGKVTVKTLDKRWDASVAIATLKII |
| | KQPSGYYLQLIGELPTKKFKPSNKAVGISLGYKDLFTTDGGKVVKSPLYYQKMEKKLQRLQRKLCRQQ |
| | NLCPIDTYNPSLREHFLSCPINPYKGANKAKTTQKISGLHEKIRRARRAFNHKLSTLLVQEYGGIATAKSD |
| | MRRITRRPKPIVNKEGTGYDRNGAERKSQFNKLILANGLGQLATLIEQKAVANGREYIEVVPKDIPDEPR |
| | QRTEHESKRLRLPRAVHLSSFQSGRYRAWSWKSKPGESQWTQNQEAAQVATLRDTETTILTSSNLALER |
| | EGMDVPPTSSPKNNANQHSCRLVTTSGEKSTRATSTGQSVTEPAKTRLDEKEMPDQPEKAQRQSEVLLT |
| | AKTQVRRRKRRTAGENDSS |
| 84 | MRTVEFKLSLNRYQQAKVDSWLTIQRWVWNQGLHLLEESNSFSTWDKVSQSWVPCCSIPWTYYRDSV |
| | GQLIPFTRIAKKKPYRMSCPIPQAYRKPLLETPTFFGLLYYFAQKNHSDKPWFCDVPCRFVAGTLKSLAD |
| | AWTAYKSGKRKRPRYKQYKDKFRTLTNNNAKPVKISGKRITLPKLGKVTVKTLDRRWLKSVPIVTLKIV |
| | KEPSGYYLQLTGCFPVNKVKPTNKAVGVSLGYSHLTTDGEKVVEPPNFYHKMEKQLAQLQRQLCRQQ |
| | KTCPIFSYNPSLGEHFLSCSINPSKGANRAKTQRKISRLHEKIRRSRRATNHKISTYLVREYDAIAMVKPEI |
| | RKIARKPIAIVNKLGEFEHNGANHKAEFNKGLLDNSLGQLTSLINQKASVQGRELISVSPKDLPDELKQRT |
| | EKCCEQLQWSRAVYLTSFSRRYRAWAWELTPGESTGTLNQEPPQGGLSCDAGTTSNFISESIGLYGVGDI |
| | PEIIPLLQNQSEANSSY |
| 85 | MRTVEFKLDLNQTQQAKVDDWLNVLRWVWNRGLHLLTEFDSFTSWDKVSKTWTPSCPIQWEYYRDD |
| | DGHLVPFTRLAQTKPYRMSCPISQAYRQPELESPNHFGLLYYFAQKNHEDKPWFCEVPAKVVAGTLKSL |
| | SDAWSEYKAGKHKRPRYKRYKDKLKTLVNNNSKSVKISGKQITLPKLGKVTVKTLDKRWDAKVPIATL |
| | KIVKEPSGYYLQLTGELPLKRFKPSNKAVGISLGYKDLFTTDSGKVVKPPAYYQKMEKNLQRLQRKLSR |
| | QQNICPISTYNPELAEHFLSCPINPHKGANKAKTQQKISQQHEKIRRARRAFNHKLSTKLVQEYGGIATAK |
| | SEVRKITRRPKPIVNKEGTGYDPNSAERKSQFNKQILANGLGQLTTLIEQKAVVNGREFIEIAPKEIPDEPR |
| | QRAERYSKRLRLPRAVHLSSFFGRYRAWSWESKPGESQRTLNQEASQEAALRDAGTTSKSSSANTNLTE |
| | SSNFNGDRASRATSQSSLETRSELANPCKSESFKQLPKAKKHSSAPPTSEKQLGRKKRRSTRENDSS |
| 86 | MIKKKAFKFLLEPSKSQISDVLVFAGACRFVYNKGLALLSENYNNGKPFLNYNKLAPLLVEWKNDNKLE |
| | WLKLCPSQCLQQSLRDLDRAFQNFFSGRSQYPRFKKKGRSDSFRVPCQRVRLDQEKGLVSLPKLGWVK |
| | YRKSRAITGDLKNVTVSRKFDKWYISFNTEEVVSNPVHPSVDKTRILLNDGYVTLCTGGDLSVKKFTSLV |
| | DEKKIKRLNKELSRKVKNSNNWLKNKKKIDKIRLKSGSFRLDAIHKITTTICKKHAVVEVVNVKNFVSD |
| | KNNIATSMRYELVRQLLYKQEWLGGKIIHLDA |
| 87 | MIKKQAFKFLLEPNKNHINEFLVFAGSCRFVYNKGLALINENYDSGKKFLNYNQLASELVNWKNEECLA |
| | WLKMAPSQCLQQSLRDLDKAFKNFFSGKSQYPRFKKKGRNDSFRVPCQRVRLDQEKHLVSLPKLGWV |
| | KYRKSREITGVLKNVTISRKLDKWYISFNTEVVVPEPVHPSFSKAKVLLNNECIVQLTSNESLVEQFTSME |
| | GNKKLRNLNNILGRKVKYSSNWLKTKKKIDSVKARSSRRRLDALHKITTAICKKHAIVELVNLTDSLPD |
| | KNNGFVSMGYEFVRQLMYKQEWLGGQVIRLGD |
| 88 | MIKQQAFKFALKLNEQQKANMLLFAGACRFVYNKGLALLKESYESGQKHMHYNQLAPLLVEWKSDPA |
| | LSWLKQAPSQSLQQSLRDLDKAFSNFFYGNAEHPRFKKKGQHDAFRFPSQRVKVDQEKQLVLLPKLGW |
| | VKYRKSRDITGDIKNVSISGKLGKWYISFNTQTDIEEPVHPAISKIGVYVDAKKNITLSDGTQYIPPQSLITL |
| | PKQIQRLTNCLRKKNRYSNNWLKSKHRINRLSSRLNQVKVDYLHKASTAISKNHAGDAANLLI |
| 89 | MKKAYKFRMYPNKKQQELINKTFGCCRFVYNKYLAKRIEVYKNNKETFTYKQCSSDLTNLKKELNWL |
| | KEPDKFSLQNALKDLENAYEKFFKEKTGFPKFKSKKTNRFSYKTNFTNGNIMYCGQHIKLPKLGMVKIS |
| | DKQVPKGRILNATISKEPSGRYYVSLCCTDVDIEAFENTNNQIGLDLGIKEFCISSYGDFIENPKYLKKSLN |
| | KLAKLQRELSRKTIGSLNRNKARLKVARLQEHIANQRKDFLQKLSTKLIKENELIKETILFAYKIYK |
| 90 | MEKAYKFRMYPNKKQQELINKTFGCCRFVYNKYLAKRIDVYKNDKETFTYKQCSSDLTNLKKELKWL |
| | KEPDKFSLQNALKDLDNAYKKFFKEKAVFPKFKSKKINRFSYKTNFTNGNIMYCGQHIKLPKLGMVKIR |
| | DKQVPKDRILNATISKEPSGRYYVSLCCTDVDIEAFENTNNQIGLDLGIKEFCISSYGDFIENPKYLKKSLN |
| | KLAKLQRELSRKTIGSLNRNKARLKVARLQEHIANQRKDFLQKLSTKLIKENELIKETILFAYKIYK |
| 91 | MKKAYKFRMYPNKKQQELINKTFGCCRFVYNKYLAKRIEVYKNNKETFTYKQCSSDLTNLKKELNWL |
| | KEPDKFSLQNALKDLENAYEKFFKEKAGFPKFKSKKTNRFSYKTNFTNGNIMYCGQHIKLPKLGMVKIS |
| | DKQVPKGRILNATISKEPSGRYYVSLCCTDVDIEAFENTNNQIGLDLGIKEFCISSYGDFIENPKYLKKSLN |
| | KLAKLQRELSRKTIGSLNRNKARLKVARLQEHIANQRKDFLQKLSTKLIKENELIKETILFAYKIYK |
| 92 | MEKAYKFRMYPNKKQQELINKTFGCCRFVYNKYLAKRIEVYKNNKETFTYKQCSSDLTNLKKELKWL |
| | KEPDKFSLQNALKDLDNAYKKFFKEKVGFPKFKSKKINRFSYKTNFTNGNIMYCGQHIKLPKLGMIKIRD |
| | KQVPQGRILNATISKEPSGRYYVSLCCTDVDIEAFENTNNQIGLDLGIKEFCISSCGEFIENPKYLKKSLSK |
| | LAKLQRELSRKTIGSLNRNKARLKVARLQEHIANQRKDFLQKLSTKLIKETILFAYKIYK |
| 93 | MEPTREQGEALERMAGARRWVWNWGLARRKEAYAATGKGLTYNQQAALLTALKQQPETAWLKEAD |
| | SQLLQQALKDLDRAFKAFFEKRAGFPQFKSKKRDTPRFRIPQRVKVEGSKVYIPKVGRVKIRQSQPIDCP |
| | VKGATFKRDTQGHWYVTLTAEFEMPEVPLPPANPERVVGIDLGLKDFAVLSDGTRIAPPEFYRKAERRL |
| | RKAHKELSRKQKGGKNRDKARERLNRVHAKVRNQRQDWLHKLTTGLVQKYSTTGCASRT |
| 94 | MKSLADAWTAYKSGKRQRPCYKQYKDKFRTLINNNAKPIKISGKRITLPKLGKVTVKTLDRRWLKSVPI |
| | VTLKIVKEPSGYYLQLSGCFPVNKVKPTNKAVGVSLGYSHLTTDGEKVVEPPNFYHKMEKQLAQLQRQ |
| | LSRQQKTCPISTYNPSSGEHFLSCPINPGKGANRAKTQRKISRLHEKIRRSRRATNHKISTYLVREYDAIAI |
| | VKPEIKRIARKPIAIVNKLGEFEHNGANHKAEFNKGLLNNSLGQLSGLIEQKASVQGRKLISVSPKDIPDE |
| | LKQCAEKRREQIQWSRAVYSTNFSRRYRAWAWELTPGESTETLNQEPPQGGLFCDAGTTSNFISESIGFC |
| | GVGDIPEIIPLLQNQSEANSSY |
| 95 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISFNQGKWYISFNTEQTVPDPIHPSDIKTTIVLNNVNSVHLSSGVGGDNTYQAEEK |
| | KKLIRLNKTLTRRKKYSKNWLKTKGKIDRVRSKAARIRLDNIHKATTAICKNHAVVEVVNLMDSVSAK |
| | NDNTLSMRYEFVRQLIYKQEWLGGEIIRRESKLL |
| 96 | MEKAYKFRMYPNKKQQELINKTFGCCRFVYNKYLAKRIEVYKNAKETFTYKQCSSDLTNLKKELNWL |
| | KEPDKFSLQNALKDLDNAYKKFFKEKAGFPKFKSKKINRFSYKTNFTNGNIMYCGQHIKLPKLDMVKIR |
| | DKQVPKGRILNATISKEPSGRYYVSLCCTDVDIEAFENTNNQIGLDLGIKEFCISSCGEFIENPKYLKKSLN |
| | KLAKLQRELSRKTIGSLNRNKARLKVARLQEHIANQRNDFLQKLSTKLIKEXHESVRSS |
| 97 | MEKAYKFRMYPNKKQQELINKTFGCCRFVYNKYLAKRIDVYKNNKETFTYKQCSSDLNNLKKELKWL |
| | KEPDKFSLQNALKDLDNAYKKFFKEKVGFPKFKSKKINRFSYKTNFTNGNIMYCGQHIKLPKLGMVKVR |
| | DKQVPKGRILNATISKEPSGRYYVSLCCTDVDIXVVEKAYKFRMYPNKKQQELINKTFGCCRFVYNKYL |
| | AKRIEVYKNDKETFTYKQCSSDLTNLKKELKWLKEPDKFSLQNALKDLENAYEKFFKKRHDFLNLNQR |
| | KLIDFHIKLTLQMETLCIVVNI |
| 98 | MEKAYKFRMYPNKKQQELINKTFGCCRFVYNKYLAKRIDVYKNNKETFTYKQCSSDLNNLKKELKWL |
| | KEPDKFSLQNALKDLDNAYKKFFKEKAGFPKFKSKKINRFSYKTNFTNGNIVYCGQHIKLPKLGMVKVR |
| | DKQVPKGRILNATISKEPSGRYYVSLCCTDVDIEAFENTNNQIGLDLGIKEFCISSCGEFIENPKYLKKSLN |
| | KLAKLQRELSRKTIGSLNRNKARLKVARLQEHIANQRNDFLQKLSTKLIKEXSSGKSL |
| 99 | MIAVKKAYKFRMYPNKKQQELINKTFGCCRFVYNKYLAKRIEVYKNDKETFTYKQCSSDLTNLKKELK |
| | WLKEPDKFSLQNALKDLDNAYKKFFKEKAGFPKFKSKKINRFSYKTNFTNGNIMYCGQHIKLPKLGMV |
| | KIRDKQVPQGRILNATISKEPSGRYYVSLCCTDVDIEAFENTNNQIGLDLGIKEFCISSCGEFIENPKYLKKS |
| | LNKLAKLQRELSRKTIGSLNRNKARLKVARFQEHIANQRKDFLQKLSTKLIKEND |
| 100 | MIKKKAFKFLLEPSKSQISDVLVFAGACRFVYNKGLALLSENYNNGKPFLNYNKLAPLLVEWKNDNKLE |
| | WLKLCPSQCLQQSLRDLDRAFQNFFSGRSQYPRFKKKGRSDSFRVPCQRVRLDQEKGLVSLPKLGWVK |
| | YRKSRAITGDLKNVTVSRKFDKWYISFNTEEVVSNPVHPSVDKTRILLNDGYVTLCTGDDLSVKKFTSLV |
| | DEKKIKRLNKELSRKVKNSNNWLKNKKKIDKIRLKSGSFRLDAIHKITTTICKKHAVVEVVNVKNFVSD |
| | KNNIATSMRYELVRQLLYKQEWLGGEIIHLDA |
| 101 | MNVIRQKHHSTPIHIIDHEHRFVSMQHLDGFFKLRDQIDYRALPAQANQNVLHMLYRDWKSFFAALAD |
| | YKAHPDKYEAIPHIPRYADKDGYKPLIFTNQICKLRKDKHGWYVKFPKAVLQAGCVRDRYDLGKMDLH |
| | EQKLKEVRLIPNGDTIKLEIVCEIEIMEPTITIHEATRVAGIDIGVDNLTAIAFTSGHRPVLIKGNEIKAVNQF |
| | YNKQIAHYRSLLRTGKKDSKGIHQTKRMKRISEKRNRRVKDILHKASRKIIDLCVXWTPPCTDKRK |
| 102 | MNVIRQKHHSTPIHIIDHEHRFVSMQHLDGFFKLRDQIDYRALPAQANQNVLHMLYRDWKSFFAALAD |
| | YKAHPDKYEAIPHIPRYADKDGYKPLIFTNQICKLRKDKHGWYVKFPKAVLQAGCVRDRYDLGKMDLH |
| | EQKLKEVRLIPNGDTIKLEIVCEIEIMEPTITIHEATRVAGIDIGVDNLTAIAFTSGHRPVLIKGNEIKAVNQF |
| | YNKQIAHYRSLLRTGKKDSKGIHQTKRMKRISEKRNRRVKDILHKAS |
| 103 | MNVIRQKHHSTPIHIIDHDHRFVSMQHLDGFFKLRDQIDYRALPAQANQNVLHMLYRDWKSFFAALAD |
| | YKAHPDKYEAIPHIPRYADKDGYKPLIFTNQICKLRKDKHGWYVKFPKAVLQAGCVRDRYDLGKMDLH |
| | EQKLKEVRLIPNGDTIKLEIVCEIEIKEPTITIHEATRVTGIDIGVDNLMAIAFTSGHHPVLIKGNEIKAVNQ |
| | YYNKQIAHYRSLLRTGKKDSKGIHQTKRMKRISEKRNRRVKDILHKASRKIINLCVEEGIEVIVVGNNAG |
| | WKKRIHMGKKNNQTFVQIPFRTLIEMIKYKGEAAGIRVVVCEEAIQSKASSIDEDQIPVYGNDVAHTFTG |
| | KRIKRGLYRSKWHSNECRYQWSKQYHTKSISMYARARAME |
| 104 | MIKKQAFKFLLEPNKTHMNDFLVFAGSCRFVYNKGLALINENYDSGKKFLNYNQLASELVNWKNEECL |
| | AWLKMAPSQCLQQSLRDLDRAFKNFFSGKSQYPRFKKKGRNDSFRVPCQRVRLDQEKHLVSLPKLGW |
| | VKYRKSREITGVLKNVTISRKLDKWYISFNTEEVVPEPLHPSFSKTKILLNNEWLMQLTACESLVEQFAN |
| | MEGNKKLRNLNNILGRKVKYSSNWLKTKKKIDGVKARSSRRRLDALHKITTAICKKHAIVELVNLKDSL |
| | PDKNNGSVSMTYEFVRQLMYKQEWLGGKVIRLGD |
| 105 | MCPLLGREAQYIMRTVEFKLSLNRYQQAKVDSWLTIQRWVWNQGLHLLEEFNSFSTWDKVSQSWVPC |
| | CSIPWTYYRDSVGQLIPFTRIAKKKPYRMSCPIPQAYRKPLLETPTFFGLLYYFAQKNHSDKPWFCDVPC |
| | RFVAGTLKSLADAWTAYKSGKRKRPRYKQYKDKFRTLTNNNAKPVKISGKRITLPKLGKVTVKTLDRR |
| | WLKSVPIVTLKIVKEPSGYYLQLTGCFPVNKVKPTNKAVGVSLGYSHLTTDGEKIVEPPNFYHKMEKQL |
| | AQLQRQLCRQQKTCPIFSYSPSLGEHFLSCPINPSKGANRAKTQRKISRLHEKIRRSRRATNHKISTYLVRE |
| | YDAIAMVKPEIRKIARKPIAIVNKLGEFEHNGANHKAEFNKGLLDNSLGQLTSLINQKASVQGRELISVSP |
| | KDLPDELKQRTEKCCEQLQWSRAVYLTSFSRRYRAWAWELTPGESTGTLNQEPPQGGLSCDAGTTSNFI |
| | SESIGLYGVGDIPEIIPLLQNQSEANSSY |
| 106 | MLEPNKGQLSDFLAFAGSCRFVYNKGLALLNENYRSGKKFIGYNQLASELVEWKNEESLSWLKEAPSQ |
| | CLQQSLRDLDRAFRNFFTGKSQYPKFKKKGRHDSFRIPCQRVRVDQEKKLVSLPKVGWVKYRKSREIIG |
| | DLKNATISFNQGKWYISFNTEQTVPDPIHPSEIKTTIVLNNVNSVHLSSGVGGDNTYQAEEKKKLIRLNKT |
| | LTRRKKYSKNWLKTKGKIDRVRSKAARIRLDNIHKATTAICKNHAVVEVVNLMDSVSAKNDNTLSMRY |
| | EFVRQLIYKQEWLGGEIIRRESKLL |
| 107 | MLEEVAWPDALSVALPSAGRRAGMRTLEFKLYLKAEQQKLVDSWLTDLRGVWNAALDLLLEHAAFRA |
| | WDRLEKSWVPCCPLPWKFRYRPNPEGEGYIAVAYSQAARVRPWAQFCPLPQDYRVPRLESPGEFTLAA |
| | EFAHKRRPWLAHIPANLIRGVIASLVAAWERHKKDPKNCGEPKFKRPGRGDLDTLIHGDPKGAKIQPGV |
| | LPKRHPEIADARLRKRKIAFPGLGVLHARGLEHWPAEVPVCMVKITRRPSGYYLQLTGELPDSWQPKDA |
| | RPKERATAIAFDPPKQHHADDTGRVVSAPAFLQPKLDRLAKLQRKADRQQPGSNRQKRTYHRIGKLHE |
| | QIRLARRNYNQKLSTFAVRKAGALAVAQIQPALKVKTRRPKPVPSKKGLGTFDPNGAQQKSAFNLRLIDI |
| | ALGQFVALLEAKAKSRGREFQRAVNAPAASIREKGLVSWSRVYPGWAGSTDAEGGESSPAKRQGEQSP |
| | PGGVPATVTSTSSTKQNSSSSGPSGTTGANKGQKPDSKRTLQSRKAKQVLENAESQVQNASSAVDPPQE |
| | QYQIDPQSPRARRSTKKSAEDGSGSDFRAPP |
| 108 | MRDQIDYRALPAQANQNVLHMLYRDWKSFFAALADYKAHPDKYEAIPHIPRYADKDGCKPLIFTNQIC |
| | KLRKDKHGWYVKFPKAVLQAGCVRDRYDLGKMDLHEQKLKEVRLIPNGDTIKLEIVCEIEIKEPTITIHE |
| | ATRVAGIDIGVDNLTAIAFTSGHHPVLIKGNEIKAVNQFYNKQIAHYRSLLRTGKKDSKGIHQTKRMKRI |
| | SEKRNRRVKDILHKASRKIIDLCVEEGIEVIVVGNNAGWKKRIHMGKKNNQTFVQIPFHT |
| 109 | FKLRDQIDYRALPAQANQNVLHMLYRDWKSFFAALADYKAHPDKYEAIPHIPRYADKDGCKPLIFTNQI |
| | CKLRKDKHGWYVKFPKAVLQAGCVRDRYDLGKMDLHEQKLKEVRLIPNGDTIKLEIVCEIEIKEPTITIH |
| | EATRVAGIDIGVDNLTAIAFTSGHHPVLIKGNEIKAVNQFYNKQIAHYRSLLRTGKKDSKGIHQTKRMKR |
| | ISEKRNRRVKDILHKASRKIIDLCVEEGIEVIVVGNNAGWKKRIHMGKKNNQTFVQIPFHT |
| 110 | MNVIRQKHHSTPIHIIDHEHRFVSMQHLDGFFKLRDQIDYRALPAQANQNVLHMLYRDWKSFFAALAD |
| | YKAHPDKYEAIPHIPRYADKDGYKPLIFTNQICKLRKDKHGWYVKFPKAVLQAGWVRDRYDLGKMDL |
| | HEQKLKEVRLIPNGDTIKLEIVCEIEIMEPTITIHEATRVAGIDIGVDNLTAIAFTSGHRPVLIKGNEIKAVN |
| | QFYNKQIAHYRSLLRTGKKDSKGIHQTKRMKRISEKRNRRVKDILHKASRK |
| 111 | MLEPSKSQISDVLVFAGACRFVYNKGLALLSENYNNGKPFLNYNKLAPLLVEWKNDNKLEWLKLCPSQ |
| | CLQQSLRDLDRAFQNFFSGRSQYPRFKKKGRSDSFRVPCQRVRLDQEKGLVSLPKLGWVKYRKSRAITG |
| | DLKNVTVSRKFDKWYISFNTEEVVSNPVHPSVDKTRILLNDGYVTLCTGDDLSVKKFTSLVDEKKIKRL |
| | NKELSRKVKNSNNWLKNKKKIDKIRLKSGSFRLDAIHKITTTICKKHAVVEVVNVKNFVSDKNNIATSM |
| | RYELVRQLLYKQEWLGGEIIHLDA |
| 112 | MRTVEFKLSLNRYQQAKVDSWLAIQRWIWNQGLHLLEEFNSFSTWDKVSQTWVPCCPIPWTYYRDSVG |
| | QLIAFTRIAKKKPYRMSCPIPQVYRKPVLESPTFFGLLYYFAQKNHSDKPWFCDVPCRFVAGTLKSLADA |
| | WTAYKSGKRQRPRYKQYKDKFRTLINNNAKPIKISGKRITLPKLGKVTVKTLDRRWLKSVPIVTLKIVKE |
| | PSGYYLQLSGCFPVNKEKPTNKAVGVSLGYSHLTTDGEKVVESPNFYHKMEKQLAQLQRQLCRQQKTC |
| | PISTYNPSLGEHFLSCPIDPGKGANRAKTQRKISRLYEKIRRSRLATNHKISTYLVREYDAIAIVKPEIKRIT |
| | RKPIAIVNKLGEFEHNGANHKAEFSKGLLDNSLGQLAGLIKQKASVQGRELISVSPKDLPDELKQCTEKR |
| | REQLQWSRAVYSTNFSRRYRAWEWELTPGESTETLNQEPPQGGLSCDAGTTSNFILESIGLCGVGDIPEII |
| | PLLQNQSEANSSY |
| 113 | MIKKKAFKFLLEPSKSQISDVLVFAGACRFVYNKGLALLSENYNNGKPFLNYNKLAPLLVEWKNDNKLE |
| | WLKLCPSQSLQQSLRDLDRAFQNFFSGRSQYPRFKKKGRSDSFRVPCQRVRLDQEKGLVSLPKLGWVK |
| | YRKSRAITGDLKNVTVSRKFDKWYISFNTEEVVSNPVHSSVDKTRILLNDGYVTLCTGGDLSVKKFTSLV |
| | DEKKIKRLNKELSRKVKNSNNWLKNKKKIDKIRLKSGSFRLDAIHKITTTICKKHAVVEVVNVKNFVSD |
| | KNNIATSMRYELVRQLLYKQEWLGGKIIHLDA |
| 114 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEIPPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDMQDQQKLERLSNEMSS |
| 115 | MDDQAHAARTMWNCLHDWWTMLPKEKRSLAAADAATRQARKEIDWLGVLPAQAAQAVLNTYFQV |
| | WRNCWDGRADEPNFKARSRTVMSVDIPQGRDLNISRVHRRWGMVQIPRIGRIRFRWTKDLPVGKRANT |
| | ENLITGARLVKDALGWHIAFRYDQIKQLRARATRRAVDWQHKTTTDIARQYGTVVVEALTITNMVNSA |
| | KGTIEEPGKNVAQKSGLNRSISQEAWGRTVTMLTYKTARQGGTLVKVPAPGTSQRCSACGFTTPGSRQA |
| | SPWRQARRRKVGRNLPRLRGRALQGGEPDAAEAVGEETGRRAQSSTGDVPVHHGGEVRSVRGGAGRL |
| | RRGRRAPGDGAGARRRARAAARGGLGGGSRGRRAA |
| 116 | MKVLKGYRFRIYPDEEQLTFFRQTFGCVRFTYNQLLMARKNTANSEESMKLTPAVLKKDYPFLKKTDSL |
| | ALANAQRNLERAYANFFQGRASYPKLKNKKSTWQSYTTNNQKHTIYFVDEKLKLPKLKSLIQVHQHREI |
| | KGLIRSATISAKNNEEFYVSLLCLEEVTALPKTKKAIGISYCPKHLIHVSKPLDHLETIEEQMQEDRLIKAK |
| | RKLLLRAKIAKKHKVKLKDAKNYQKQKQKVHKLIQEKAFRKKDFIDQLTFSLVKEFDYIFVEKQPSTVD |
| | SEETSLFNSSDWYLFMQKLTYKTQWYGKKYLAIEKPANTENSGQMIEELGKQRLGL |
| 117 | MAEQIEEVPAELIQTRVYELHPNKTMRRVLDEACDYRRYCWNQGLALWNEMYKARQTLKSSLSTDSK |
| | KLTEEQKVLLKDKPSPSERRVRNMLVADKKDWQYAQSARILQLAISDLGKAWNNFFDKAQPGWGKPK |
| | FRSKREARQGFKSDQSKIKDGILYLERAKESSVPKDQWRGFKLSEKPLSDEFGVVSYFKEKGRYYVAIPY |
| | KIKAKDIKLPDKTGKATAVDVNVGHFDYTGGRINVLPKKLDKIYGKIKHYQRQLAKKQVKNGEAACES |
| | ENYLKTKAKLQACYRKASNIQNDLMQKFTTELVNN |
| 118 | MLKAYRYRIYPNKEQEIQLAKTFGCCRFVYNQTLAYRKDAYEKEKKSVSKTDCNNYCNRELKKAYEW |
| | LKEVDKFALTNAIYNMDSAYQKFFKEHTGYPKFKSKHDNHKSYTTNFTNGNITVDFDRGRIKLPKLKRV |
| | KIKLHRKFLGQIKAATISKVPSGKYYVSVLVETEHSPLVKTNGQIGLDLGIKDLCITSDGKKYENPKTIKK |
| | YEKKLVKLQRQLANKIKGSGTIRKKGNKSTMHEKITNTRKDYLHKISSEIINENQV |
| 119 | MLKAFKFRMYPTEEQKQQLIRTFGCVRFTYNHLLKERQKSWQQTGVADFSLTPATLKKEYPFLKEVDSL |
| | ALANAQLNLDRAFRNYFKGRASFPKLKTKKSMWQSYTTNNQTHTVYLKNGHLKLPKQKELIKINQHRP |
| | VEGTIRSATISARYNEEFYVALLCDVSSIKKESSAKWIGIAYHPKTLIETSQPIEVTLPKFDQTEEKLQHAQ |
| | RKLSVKVRSAHHRKTRLDQASNYQKQKRKVMDLYLKQKNQREDYLEQLSGKLVKQYDYLFVESFPKE |
| | EAHADFSIHDWHKLITKLRYKSQWYNKKFLFINTDGAEESNSVRKSQVLEQLGRHSVIKE |
| 120 | MLKAFKFRMYPTEEQKQQLIRTFGCARFTYNHLLKKRQKSWQQTGIANFSLTPATLKKEYPFLKEVDSL |
| | ALANAQLNLDRAFRNYFKGRASFPKLKTKKSMWQSYTTNNQTRTIYLENGYLKLPKQKELIKINQHRPV |
| | EGSIRSATISARYNEEFYVALLCDVSPVKKESLAKWIGIAYHPKTLIQTSRPLEVTLPKFHQTEEKLQHAQ |
| | RKLNVKVRSAHHRKIRLDQASNYQKQKRKVMDLYLKQKNQREDYLEQLSGKLVKQYDYLFVESFPKE |
| | EAHADFSIHDWHKLITKLRYKSQWYNKKFLLINTDGAEESNSVRKSQVVEKMGRHSVIKG |
| 121 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSVFYIKEYDVIEIVEPEDRSCAEDA |
| | LFTSNEWHQLVRLLKYKAQWYGKEIQIINCQNI |
| 122 | MLKAFKFRIYPTESQKQWLIQTFGCVRFTYNHLLKARQAYYLETKEIDYTLTPASLKKQYPFLKEVDSLA |
| | LANAQLNLDRAFRNYFKGRASFPKLKNKKSIWQSYTTNNQKGTIYLEETSIKLPKLKEKIRIHAHRPIEGT |
| | IRSATISSRYNEIFYVSLLCEVPQKTMEASNKWIGIAYDPDRLVEMSTPLDIAIPKFKQVDQQLQRAKRKL |
| | VIKGRAAQHRRTHVERVKNYQKQKRKIKDLYLKQKFQREDYLEQISGTVIRHYDYLFVESISADCPEGD |
| | FSIQDWHKLLAKLQYKSQWYSKKLVLIDMKEQTDPSTNKKSLELVEIGKQVLFE |
| 123 | MFLRKAATTEGIISEGRQVPIKTLKAYRFALYPDEAQKHFFIQTFGCVRFTYNMLLTLRQQESGKTVEER |
| | TSARLQKQKMTPAKLKKDYPFLKATDSLALANAQRNLEKAFQNYYRGRASYPKLKSKKSAWQSYTTN |
| | NQGHTIYLAEDGLKLPKLKSKVLVHQHRSVAGKIRSATISAKNRQEFYVSLLCEEDIPALPKTGSEIEIAY |
| | DPTGLVVTNKPIVGIPTFCQTQVLEKLKKAQRRLSCRAKSAQRRNAKLEQAKNYQKQKSQVQQLYIHKL |
| | KQKEDFTEQLSIALLRQFDCIIITKPPELRENKESKAAKTVKKSKHTTVFPSFEDNFTLSDWNRLLLKLKY |
| | KAEWYEKELVFICPTNGK |
| 124 | MSVLKGYKFRIYPDEKQKKFFIETFGCVRFTYNHLLMARHTGTARNTTLTPASLKKEYPFLKKTDSLAL |
| | ANAQRNLERAFRNYFSGRAGYPKLKTKKSTWQSYTTNNQQHTVYLEGEYLKVPKLKSLVPIHLHREVR |
| | GTIKSVTISAKRNREFYASILCVEEVEELPKTNDLVGISYCPENLIQISAKKELPQIDQSHLVKQLGKEQKK |
| | LQLRAKVAKKRKVRLIHAKNYQKQKERVLKLRATKLDQKRNFIDQLTINLVRDFDYLFIESKPKFKNET |
| | GEFSEADWQQFIQRIQYKGRWYGKEIRYIEVKELKNEKCKEIERLGRAQLT |
| 125 | MKILKGYRFKIYPNEEQKRFFIQTFGCVRFTYNYLLKAAKKPDNRSEGKVITPAMLKRDYPFLKATDSLA |
| | LANAARNLNRAFKNYFSGRSGYPKLKNKKSAWQSYTTNNQNGTVAIEGNQLKLPKLKERVMICCHRPV |
| | LGTIKSVTISAKNNQEFYVSLLCVEEVDPLPKTNREIQIYFHPEKLIADDLGQLSIQHLEQTQQKINKLTQR |
| | LELKARCARKRKVRLSQAKNYQKLKMRLAKHQSLQHNQLQDYLNQLSTLLIRKYDVINFVEPSVRDQQ |
| | SAANVQEAHLFSLNEWHQLMRMLKYKASWYGKEFKVVFSTQA |
| 126 | MNVLKGYRFRIYPNKEQQEFFTQTFGCVRFVYNHLLMARKEEHYSAESLKLTPASLKATYPFLKKTDSL |
| | ALANAQRNLDRAFLNFFKGRAGYPNLKSKKKTWQSYTTNNQKHTIYFEEGKLKVPKLKTLIDVHQHRE |
| | VKGQIKSATISAKNSEEYYVSLLCLEEITALPKTKKVVGVAYCPKHLVSVSCGREHLPELTKSTVEERLA |
| | RARQKLELRAKIVKKRKVHRDCAKNYQKQKRRVDKLYLTRAYQKNDYIDKLTLKLIEQYDYVFLEKEP |
| | NFEKNCSFTETDWHVFMQKLRYKGKWYGKELRLIDIASDQEEKSETLEHLGRTQFSK |
| 127 | MLKAFKFRMYPTEEQKQQLIRTFGCARFTYNHLLKKRQKSWQQTGVADFSLTPATLKKEYPFLKEVDS |
| | LALANAQLNLERAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIAYHPKTLIQTSRPLEVTLPKFHQTEEKLQHA |
| | QRKLNVKVRSAHHRKTRLDQASNYQKQKRKVMDLYLKQKNQREDYLEQLSGKLVKQYDYLFVESFP |
| | KEEAHADFSIHDWHKLITKLRYKSQWYNKKFLLINTDGAEESNSVRKSQVVEEMGRHSLIKG |
| 128 | MAKFEIPEGWMVQAFRFTLDPTAEQARALARHFGARRKAYNWTVATLKADIDAWQATGIQTAKPSLR |
| | VLRKRWNTVKNDVCVNIETGVVWWPECSKEAYADGIDGAVDAYWNWQNSRSGKRDGKRMGFPRFK |
| | KKGRDPDRVTFTTGAMRVEPDRRHLTLPVIGTVRTHENTRRVERLIAKGRSRVLAITVRRNGTRIDASVR |
| | VLVQRPQQPKVTDPGSRVGVDVGVRRLATVATADGAVLERVPNHGLDDLQRLGKRGDSVANRRERDA |
| | VGLRLAPKPAGAQSQIETSLGNDVQRRSHLGQHGRMTVGIAQHAGAQPQLRGVAGQCRKCGPALEQG |
| | QRPRRGALARPAGFGAGRGAGVGGDAGDLRILAGAYRQEVVA |
| 129 | MPKFEVPDGWTVQAFRFTLDPTEDQAKALARHFGARRKAYNWTVATLKADIQAWHASGTVTAKPSLR |
| | VLRKRWNTVKDDVCVNTETGVAWWPECSKEAYADGIAGAVEAYWNWQTSRAGKRAGKRVGFPRFK |
| | RKGRDQDRVSFTTGAMRVEPDRRHLTLPVIGTVRTHENTRRIERLIKAGRARVLAISVRRNGTRLDASVR |
| | VLVQRPQQPKVVHPGSRVGVDVGVRRLATVATADGTAIEQVENPRPLGAALRELRHVCRARSRCTKGS |
| | RRYRERTTQISRLPGQRCPHPSPARPDDTVGSNPRPHCCRRLGRDRDVAAKRVAGCPRSSARTVGCGPG |
| | HSASALVLQDSLVRVGAGGRRPLVPVVENLPRLPACARHRLGRTMAMRPMLSGPSA |
| 130 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 131 | MKNLKGYRFRIYPNEAQKRFFIETFGCVRFIYNYFLKLDTAERTSEEIITPASLKRDYPFFKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 132 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARNQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 133 | MNLAAWAERNGVARVTAYRWFHAGLLPVPARKVGRLILVDELASEAGAQPKTAVYARVSSADQKSDL |
| | DRQVARVTSWATAEQIPVDKVVTEVGSVLNGHRRKFPAVLRDLSVTRIVVEHRDRFCRFGSEYVHAAL |
| | AAQGRELVVVDSAEVDDDLVWDMTEILTSMCARLYGKRAAQNRASGPSRLPLSMIMRRPEMPRLEIPN |
| | GWCVQAFRFTLDPTAEQAHALARHFGARRKAYNWTVAQLKADIQAWRATGAQTAKPSLRVLRKRWN |
| | TVKDEVCVNAETGTVWWPECSKEAYADGIAGAVDAYWNWQQRRAGKRDGKRMGFPRFKKKGRDAD |
| | RVSFTTGAMRVEPDRRHLTLPVIGCVRTHENTRRIERLIAKDRARVLAITVRRNGTRLDASVRVLVQRPQ |
| | QPNVELPESRIGVDVGVRRLATVATADGACCPVLVPDG |
| 134 | MKLSVLKAYKFRIYPNGQQKQFFIETFGCVRFTYNQLLKAKMDELANKEVKLGLTPAKLKKDYPFLKET |
| | DSLALANAQRNLERAFRNYFQKRAGFPKMKTKKSVWQSYTTNNQQHTIYFVDDQLKLPKLKSLVPVKL |
| | HREIKGTIKSATISAKNGTEFYVSILCLEEVEPLPKQQQNIALIFDPQILVQANHSLPVACTHALSTLQKLV |
| | KAENKLTIKAKAVKRKKILLNNARNYQKQKGKVAKLYRLHGCQKREYIDQMSYHLVKQYDTIFIEKINE |
| | DMDVAGNYSVSDWHQFIRKMQYKAKWYGKELHFVPLSATENQKMTELLSQMGS |
| 135 | MEKAYKFRMYPNKKQQELINKTFGCCRFVYNKYLAKKIEVYENNKETFTYKQCSSDLTNFKKELEWLK |
| | EPDKFSLQNTLKDLENAYKKFFKENAGFPKFKSKKTNRFSYRTNFTNENIMYCGQYIKLPKLGMVKVRD |
| | KQVPQGRILNATISKEPSGKYYVSLCCTDIDIKAFENTNNQIGLDLGVKEFCISSCGDFIENPKYLKKSLNK |
| | LAKLQRELSRKTIGSLNRNKARLKVARLQEHIANQRKDFLQKLSTKLI |
| 136 | MKVLKAYKFRLYPSAEQKAFFIFTFGCVRFTYNHLLKERQQEYQRTGFLGKGRTPAQLKKEFPFLKKTD |
| | SLALANAQLNLDRAYRNYFRSQAGFPKLKTKKSLWQSYTTNNQQGTIDLVDGQLKLPKLKEHIPVLVH |
| | RAVKGKIKSATISAKYNEIFYVSLLCEEEVAPLPKTEKQVAVVFCQEIGIRTSQKILYPPYAVAGLESSLAK |
| | AERRLQIKATSARKRKVKLMDARNYQKQKRRVAQLYQIRYQRKRDYLEKLSFELVQAFDVIFIGKDSIQ |
| | EQPGPFDQQDWLLFLQKLAYKAKWYQKQLVFVEVPRLLQDPSELERTGTALLNKPNWQGRQGSPRE |
| 137 | MKKEDLVKVLKGYKFRIYPDEKQIQYFIQTFGCVRFTYNQLLLARQKALQEGEYKTDVSPAKLKLDYPF |
| | LKKTDSLALANAQRNLDRAFKNYFSKRAGYPKLKTKKNSWQTYTTNNQKHTIYFVGNQLKLPKLKTLI |
| | NVNLHREVLGEIKSATISAKDNQLFFVSILCLEEVTPLPKTGKSIAISYCPKHLVQIPATNYLPAFRQEKLQ |
| | WQLDKAMKRLKVRAKAAKNRKVLLEKAKNYQKQKVKVQKLYMAKNEQKKNYMNQVSYRLVRDYD |
| | YIYLEKTPTFMENMNFSETDWHHFLRKLQYKVQWYGKKIIFVDAVENVRTKENSPLNV |
| 138 | MEKAYKFRMYPNKKQQELINKTFGCCRFVYNKYLAKRIDVYKNDKETFTYKQCSSDLTNLKKELNWL |
| | KEPDKFSLQNALKDLENAYNKFFKEKAGFPKFKSKKINRFSYKTNFTNGNIMYCGQHIKLPKLGMVKIR |
| | DKQVPKGRILNATISKEPSGRYYVSLCCTDVDIEAFENTNNQIGLDLGIKEFCISSCGEFIENPKYLKKSLN |
| | KLAKLQRELSRKTIGSLNRNKARLKVARLQEHIANQRNDFLQKLSTKLIKE |
| 139 | MEKAYKFRMYPNKKQQELINKTFGCCRFVYNKYLAKRIEVYKNDKETFTYKQCSSDLTNLKKELKWL |
| | KEPDKFSLQNALKDLDNAYKKFFKEKVGFPKFKSKKINRFSYKTNFTNGNIMYLGQHIKLPKLGMIKIRD |
| | KQVPQGRILNATISKEPSGRYYVSLCCTDVDIEAFENTNNQIGLDLGIKEFCISSCGEFIENPKYLKKSLNK |
| | LAKLQRELSRKTIGSLNRNKARLKVARLQEHIANQRKDFLQKL |
| 140 | MLKLRQQNPSDESTLPEKMTGVWEKKTTATPAKLKRDYPFLKETDSLALANAQRNLTKAFQNYYRGR |
| | ASYPKLKSKKNAWQSYTTNNQGHTIYLTNEGLKLPKLKSKVPIHQHRQVCGKIRSATISAKNRQEFYVS |
| | LLCEEEITALPKTGFDITITYDPIKLIGTSKVLSDRPNFCQQRLLVQLKNAQRKLYCRGKSAQRRNVKLEQ |
| | AKNYQKQKLRLQKLYIHQIKQKEDFMEQLSIALLRQFDLVTVTMPKAFESLSANHSAAIHQDCSANYKN |
| | TAVNFTIRDWNRFVLKLKYKANWYGKKLIFTDQEKVI |
| 141 | MSVLKAYRFRIYPNEEQKHFFVTTFGCVRFTYNHLLVARQQSEGGKLTPAALKKDYPFLKATDSLALAN |
| | AQRNLEKAFRRYYTGKSDYPSLKNKSNPLQSYTTNNQGQTICLSDGYLKLPKLKSLVAVNCHREIKGTI |
| | KSATISSRNNEEFFVSFLCVEEVEPLPKTLKTIHLVYSPNKLLESSEYTPPTLCNQEQLLDKIDRAQRKLRV |
| | RGKIARKRRVPLAYAKNYQKQKEKLGRLQLSCREKKENYFDQVSYAIVRQFDFIHVTKELLFETLPDQP |
| | LYFSKADWQMFLKKLEYKAEWYGKELTYE |
| 142 | MVVVEKAYKFRMYPNKKQQELINKTFGCCRFVYNKYLAKRIDVYKNNKETFTYKQCSSDLTNLKKEL |
| | KWLKEPDKFSLQNALKDLDNAYKKFFKEKAGFPKFKSKKINRFSYKTNFTNGNIMYCGQHIKLPKLGM |
| | VKIRDKQVPKGRILNATISKEPSGRYYVSLCCTDVDIEAFENTNNHIGLDLGIKEFCISSCGEFIENPKYLK |
| | KSLNKLAKLQRELSRKTIGSLNRNKARLKVARLQEHIA |
| 143 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEIPPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 144 | MKKAYKFRLYPNKKQQELINKTFGCCRFVYNKYLAKRIDVYKNNKETFTYKQCSSDLTNLKKELKWLK |
| | EPDKFSLQNALKDLDNAYKKFFKEKAGFPKFKSKKINRFSYKTNFTNGNIMYCGQHIKLPKLGMVKVRD |
| | KQVPKGRILNATISKEPSGRYYVSLCCTDVDIEAFENTNNQIGLDLGIKEFCISSCGEFIENPKYLKKSLNK |
| | LAKLQRELSRKTIGSLATYSHFIFSFFSYYNGFDKSVINFYK |
| 145 | MIAVKKAYKFRIYPNKKQQELINKTFGCCRFVYNKYLAKRIDVYKNDKETFTYKQCSSDLTNLKKELN |
| | WLKEPDKFSLQNALKDLDNAYKKFFKEKAGFPKFKSKKINRFSYKTNFTNGNIMYCGQHIKLPKLGMV |
| | KVRDKQVPKGRILNATISKEPSGRYYVSLCCTDVDIEALENTNNHIGLDLGIKEFCISSCGEFIENPKYLKK |
| | SLNKLAKLQRELSRKTIGSLNRNKARLKVARLQEHIANQRNDFL |
| 146 | MVVVEKAYKFRMYPNKKQQELINKTFGCCRFVYNKYLAKRIDVYKNNKETFTYKQCSSDLTNLKKEL |
| | KWLKEPDKFSLQNALKDLDNAYKKFFKEKTGFPKFKSKKINRFSYKTNFTNGNIMYCGQHIKLPKLGM |
| | VKIRDKQVPQGRILNATISKEPSGRYYVSLCCTDVDIEAFENTNNHIGLDLGIKEFCISSCGEFIENPKYLK |
| | KSLNKLAKLQRELSRKTIGSLNRNKARLKVARLQEHIANQR |
| 147 | MIAVKKAYKFRIYPNKKQQELINKTFGCCRFVYNKYLAKRIDVYKNDKETFTYKQCSSDLTNLKKELK |
| | WLKEPDKFSLQNALKDLDNAYKKFFKEKAGFPKFKSKKINRFSYKTNFTNGNIMYCGQHIKLPKLGMV |
| | KIRDKQVPKGRILNATISKEASGRYYVSLCCTDVDIEVFENTNNQIGLDLGIKEFCISSCGEFIENPKYLKK |
| | SLNKLAKLQRELSRKTIGSLNRNKARLKVARLQEHIANQRNDFL |
| 148 | MLKAFKFRIYPTASQKEWFIQNFGCVRFTYNHLLKARQESYARTGAIDYSMTPATLKKKYAFLKSADSL |
| | ALANAQLNLDRAFRNYFNGRASFPKLKNKKSMWQSYTTNNQKGTIYLEDKYLKLPKQKELIQVRLHRP |
| | VEGVIRSATISARYNESFYVSLLCEVQIAGVPTTNRWLGVAYDPKKLVETSSPVEVQMPLFRQTRDKMK |
| | VAKRKLVIKSKAAQKRKVRLENARNYQKQKRKVMDLYQKQKLQKEDYLERVSGNLIRNYDYLFVEAV |
| | PSELSSADFQLQDWYKLITKLRYKAQWYNKTLLFINVNEQLNEPPEKKSMELEKIGKQVIFE |
| 149 | MKLGVLKAYKFRIYPNGQQKQFFIETFGCVRFTYNQLLKAKMDELANKEVKLGLTPAKLKKDYPFLKE |
| | TDSLALANAQRNLERAFRNYFQKRAGFPKMKTKKSIWQSYTTNNQQHTIYFVEDQLKLPKLKSLVPVK |
| | QHRAIKGTIKSATISAKNGTEFYVSILCLEEVEPLPKKQQKIALIFDPQLLVQANHSLPVACTHALATLQK |
| | LARAENKLTIKAKAVKRKKILLNNARNYQKQKGKVAKLYRLHGCQKREYIDQMSYHLVKQYDTIYIEK |
| | ISEDAQVSGNYTISDWHQFVRKMQYKAKWYGKELHFVALSATDNRKMPELLAQMGS |
| 150 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEEDQIKLPKLKTLVPVKK |
| | HREIKGKIKSATISAKNNEEFYISILCLEEITPLPKQQASIAVVYDPQQLVKANQPVPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 151 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEEDQIKLPKLKTLVPVKK |
| | HREIKGKIKSATISAKNNEEFYISILCLEEITPLPKQQASIAVVYDPQQLIKANQSVPVTCEHAIQTKQKLTR |
| | AERKLQVKATAVKRKKILLTQARNYQKLKGKVARLYRFHCCQKREFIDQVSYHLVKQYDTIYLEQIAEE |
| | TVLKAGHYSISDWHQFVRKIQYKAQWYGKELRFVTLDTQDQQKLERLSGEMSS |
| 152 | MLKAFKFRMYPTEEQKQQLIRTFGCVRFTYNHLLKERQKSWQQTGVADFSLTPATLKKEYPFLKEVDSL |
| | ALANAQLNLDRAFRNYFKGRASFPKLKTKKSMWQSYTTNNQTHTVYLKSGYLKLPKQKELIKINQHRP |
| | VEGTIRSATISARYNEEFYVALLCDVSSIKKESSAKWIGIAYHPKTLIETSQPIEVTLPKFDQTEEKLQHAQ |
| | RKLSVKVRSAHHRKTRLDQASNYQKQKRKVMDLYLKQKNQREDYLEQLSGKLVKQYDYLFVESFPKE |
| | EAHADFSIHDWHKLITKLRYKSQWYNKKFLFINTDGAEESNSVRKSQVLEQLGRHSVIKE |
| 153 | MLKAFKFRIYPTDSQKQWLIQTFGCVRFTYNHLLKARQAYYLETQEIDYTLTPASLKKQYPFLKEVDSL |
| | ALANAQLNLDRAFRNYFKGRASFPKLKNKKSIWQSYTTNNQKGTIYLEETYLKLPKLKEKIRIHAHRPIE |
| | GTIRSATISSRYNEIFYVSLLCEVPQKTMKASNKWIGIAYDPDRLVEMSTPLDITIPKFKQVDQQLLRAKR |
| | KLVIKGRSAQHRRTHVERVKNYQKQKRKIKDLYLKQKFQREDYLEQISGTVIRHYDYLFVESISADCPEG |
| | DFSIQDWHKLLAKLQYKSQWYSKKLVLIDMKEQTNPSTNKKSLELVEIGKQVLFE |
| 154 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTSNEWHRLVRLLKYKAQWYGKEIQIINCQNI |
| 155 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQTKYRAEVIEPIQHTKGRLAFLQRRLK |
| | VKARVARKQNRILADCKNYQKQKKQYDKLLTHLNNQIKDYLNHLSIFYIKEYDVIEIVEPEDRPCAKDD |
| | LFTSNEWHQLVRLLKYKAQWYGKEIQIINCQNI |
| 156 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYHAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 157 | MKILKAYKFRIYPDEAQQEFFIKTFGCVRFTYNTLLKLRQQNPSDESTLPEKMTGVWEKKTTATPAKLK |
| | RDYPFLKETDSLALANAQRNLTKAFQNYYRGRASYPKLKSKKNAWQSYTTNNQGHTIYLTNEGLKLPK |
| | LKSKVPIHQHRQVCGKIRSATISAKNRQEFYVSLLCEEEITALPKTGFDITITYDPIKLIGTSKVLSDRPNFC |
| | QQRLLVQLKKAQRKLYCRGKSAQRRNVKLEQAKNYQKQKLRLQKLYIHQIKQKEDFMEQLSIALLRQF |
| | DLVTVTMPKAFESLSANHSAAIHQDCSVNYKNTAVNFTIRDWNRFVLKLKYKANWYGKKLIFTDQEKV |
| | I |
| 158 | MLKAFKFRIYPTASQKEWFIQNFGCVRFTYNHLLKARQESYARTGAIDYSMTPATLKKKYAFLKSADSL |
| | ALANAQLNLDRAFRNYFNGRASFPKLKNKKSMWQSYTTNNQKGTIYLEDKYLKLPKQKELIQVRLHRP |
| | VEGVIRSATISARYNESFYVSLLCEVQIAGVPTTNRWLGVAYDPKKLVETSSPVEVQMPLFRQTRDKMK |
| | VAKRKLVIKSKAAQKRKVRLENARNYQKQKRKVMDLYQEQKLQKEDYLERVSGNLIRNYDYLFVEAV |
| | PSELSSADFQLQDWYKLITKLRYKAQWYNKTLLFINVNEQLNEPPEKKSMELEKIGKQVIFE |
| 159 | MVVVEKAYKFRMYPNKKQQELINKTFGCCRFVYNKYLAKRIEVYKNDKETFTYKQCSSDLTNLKKELK |
| | WLKEPDKFSLQNALKDLDNAYEKFFKEKAGFPKFKSKKINRFSYKTNFTNGNIMYFSQHIKLPKLGMVK |
| | IRDKQVPKGRILNATISKEPSGRYYVSLCCTDVDIEAFENTNNQIGLDLGIKEFCISSCGDFIENPKYLKKSL |
| | NKLAKLQRELSRKTIGSLNRNKARLKVARLQEHIANQRNDFLQ |
| 160 | MIAVKKAYKFRMYPNKKQQELINKTFGCCRFVYNKYLAKRIEVYKNNKETFTYKQCSSDLTNLKKELN |
| | WLKEPDKFSLQNALKDLENAYEKFFKEKTGFPKFKSKKTNRFSYKTNFTNGNIMYCGQHIKLPKLGMV |
| | KIRDKQVPKGRILNATISKEPSGRYYVSLCCTDVDIEAFENTNNQIGLDLGIKEFCISSCGDFIENPKYLKK |
| | SLNKLAKLQRELSRKTIGSLNRNKARLKVARLQEHIANQR |
| 161 | MVVVEKAYKFRMYPNKKQQELINKTFGCCRFVYNKYLAKRIDVYKNNKETFTYKQCSSDLTNLKKEL |
| | KWLKEPDKFSLQNALKDLDNAYKKFFKEKTGFPKFKSKKINRFSYKTNFTNGNIMYCGQHIKLPKLGM |
| | VKIRDKQVPQGRILNATISKEPSGRYYVSLCCTDVDIEAFENTNNHIGLDLGIKEFCISSCGEFIENPKYLK |
| | KSLNKLAKLQRELSRKTIGSLNRNKARLKVARLQEHIANQRNDFLQK |
| 162 | MKNSSLLNKGYKFRIYPNIHQIAKIEKNFGCVRFVYNYFLSQRIRAYDSAGKTIGYLEQQNHLPLLKKQY |
| | PWLKVADSTSLQISIRNLDKAFQNFFKNKAFGFPNFKSKKSIRKCYTVNCVNSNIVIKDGKIKLPKLKWV |
| | DAKVHRKVEGRIISATVIKSSSGKYYVSVITEQKKRQIPSTEGKNIISLQMKDFITISNDKSIYYFKYIKKIK |
| | KLERKLLSKEKESNNRKKLEKQIGKLYEKIQNKRDDFLHKLSKNIVDENQIIYIQECNVKKGTDYIENCA |
| | HLKSFSWSKFCKFLEYKSCWYNKSLIYVENSDSYLNLEYNYKSLNTNIPLNLGEKINVTLMKKILRPHSF |
| 163 | MLKAFKFRIYPTESQKQWLIQTFGCVRFTYNHLLKARQAYYLETKEIDYTLTPASLKKQYPFLKEVDSLA |
| | LANAQLNLDRAFRNYFKGRASFPKLKNKKSIWQSYTTNNQKGTIYLEETSIKLPKLKEKIRIHAHRPIEGT |
| | IRSATISSRYNEIFYVSLLCEVPQKTMEASNKWIGIAYDPDRLVEMSTPLDIAIPKFKQVDQQLQRAKRKL |
| | VIKGRAAQHRRAHVERVKNYQKQKRKIKDLYLKQKFQREDYFEQISGTVIRHYDYLFVESIPADCREGD |
| | FSIQDWHKLLAKLQYKAQWYSKKLVLIDMKEQTNPSTTKKSLELVEIGKQVLFE |
| 164 | MKTLQAYRFALDLSPRQERAVLAHAGAARVAHNWALARVRAVMSQRAAERTYGVPDELLSPPISWSL |
| | PSLRKAWNAAKDEVAPWWAECSKEAFNTGLDALARALKNWSDSRKGARKGHAVGFPRFKSRRRSTPT |
| | VTTGVMRIEADRRHVVLPRLGALRLHESARKLARRLEAGTARIMSATVRREGGRWFVSFTCQVERAVR |
| | APARPGSMVGVDLGVKHLAVLSTGERVANPRHLVVAARRMRRLARAVSRCVTPDRRVRRVGSNRCPG |
| | RSKSFRGRTRASSGYAATVYTSSPPG |
| 165 | MSSCRTLDNKVDSMKNLKGYRFRIYPNEAQKRFFIETFGCVRFIYNYFLKLDTAERTSEEVITPASLKRD |
| | YPFLKKTDSLALANAKRNLDRAFQNYYQQRSGYPKLKNKSSAWQSYTTNNQNGTVRIEDGYLKLPKLK |
| | EKIQICEHRKITGKIKSVTISAKNNEEFYASILCVETIDKFEKTGKKIRLSFDEHQLVKQAKYRAEVIEPIQQ |
| | TKGRLEFLQRKLKVKARVARKQNRVLADCKNYQKQKKQYDKLLTHLNNQIKDYLNHLSIFYIKEYDVI |
| | EIVEPEDRSCAKDDLFTSNEWHQLTRLLKYKAQWYGKEIQIINCQNI |
| 166 | MSSCRTLNNKVDSMKNLKGYRFRIYPNEAQKRFFIETFGCVRFIYNYFLKLDTAERTSEEIITPASLKRDY |
| | PFLKKTDSLALANAKRNLDRAFQNYYQQRSGYPKLKNKSSVWQSYTTNNQNGTVRIEDGYLKLPKLKE |
| | KIQICEHRKITGKIKSVTISAKNNEEFYASILCVETIDKFEKTGKKIRLSFDAHQLVQQAKYRAEVIEPIQHT |
| | KGRLAFLQRRLKVKARVARKQNRILVDCKNYQKQKKQYDKLLTHLNNQIKDYLNHLSIFYIKEYDVIEI |
| | VEPEDRPCAKDDLFTSNEWHQLVRLLKYKAQWYGKEIQIINCQNI |
| 167 | MADFSLTPATLKKEYPFLKEVDSLALANAQLNLDRAFRNYFKGRASFPKLKTKKSMWQSYTTNNQTHT |
| | VYLKNGHLKLPKQKELIKINQHRPVEGTIRSATISARYNEEFYVALLCDVSSIKKESSAKWIGIAYHPKTLI |
| | ETSQPIEVTLPKFDQTEEKLQHAQRKLSVKVRSAHHRKTRLDKASNYQKQKRKVMDLYLKQKNQRED |
| | YLEQLSGKLVKQYDYLFVESFPKEEAHADFSIHDWHKLITKLRYKSQWYNKKFLLINTDGAEESNSVRK |
| | SQVLEQLGRHSVIKE |
| 168 | MKNLKGYRFRIYPNEAQKRFFIETFGCVRFIYNYFLKLDTAERTSEEVITPASLKRDYPFLKKTDSLALAN |
| | AKRNLDRAFQNYYQQRSGYPKLKNKSSAWQSYTTNNQNGTVRIEDGYLKLPKLKEKIQICEHRKITGKI |
| | KSVTISAKNNEEFYASILCVETIDKFEKTGKKIRLSFDEHQLVKQAKYRAEVIEPIQQTKGRLEFLQRKLK |
| | VKARVARKQNRVLADCKNYQKQKKQYDKLLTHLNNQIKDYLNHLSIFYIKEYDVIEIVEPEDRSCAKDD |
| | LFTSNEWHQLTRLLKYKAQWYGKEIQIINCQNI |
| 169 | MLKAFKFRIYPTTLQKQWFIQNFGCVRFTYNYLLKVRQESYAKTGAIDYTVTPASLKKKYPFLKTADSL |
| | ALANAQLNLDRAFRNYFKGRASFPKLKNKKSMWQSYTTNNQNGTIYLEKNYLKLPKQKERIKVNLHRP |
| | VEGVIRSATISARYNEVFYVSLLCEVSAQNLEGSNRWIGVAYDPQKLIETSSPLNVQLPLLKQTQDSIKIA |
| | QRKLWIKSKAAQKRKVRLEKAKNYQKQKRKVMDLYLKQKYQKEDYLEQLSGKLIRHYDYLFIEAVPN |
| | DCLSTKFSLQDWYKFIHKLRYKAHWYNKSLLLINVNEQSHLSCNQKSVTLENIGKQMMFDEMN |
| 170 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILVDCKNYQKQKKQYDKLLTHLNNQIKDYLNHLSIFYIKEYDVIEIVEPEDRPCAKDD |
| | LFTSNEWHQLVRLLKYKAQWYGKEIQIINCQNI |
| 171 | MIVVEKAYKFRMYPNKKQQELINKTFGCCRFVYNKYLAKRIDVYKNDKETFTYKQCSSDLTNLKKELK |
| | WLKEPDKFSLQNALKDLDNAYEKFFKEKAGFPKFKSKKINRFSYKTNFTNGNIMYCGQHIKLPKLGMV |
| | KIRDKQVPQGRILNATISKEPSGRYYVSLCCTDVDIEAFENTNNHIGLDLGIKEFCISSCGDFIENPKYLKK |
| | SLSKLAKLQRELSRKTIGSLNRNKARLKVARLQEHIANQRND |
| 172 | MLKAFKFRMYPTEEQKQQLIRTFGCVRFTYNHLLKERQKSWQQTGVVDFSLTPATLKKEYPFLKEVDSL |
| | ALANAQLNLDRAFRNYFKGRASFPKLKTKKSMWQSYTTNNQTHTVYLKNGHLKLPKQKELIKINQHRP |
| | VEGTIRSATISARYNEEFYVALLCDVSSIKKESSAKWIGIAYHPKTLIETSHPIEVTLPKFDQTEEKLQHAQ |
| | RKLSVKVRSAHHRKTRLDQASNYQKQKRKVMDLYLKQKNQREDYLEQLSGKLVKQYDYLFVESFPKE |
| | DAHADFSIHDWHKLIKKLRYKSQWYNKKFLLINTDGAEESNFVRKSQVLEQLGRHSVIKE |
| 173 | MEVLKAYKFRLYPTQQQRRFFIETFGCVRFTYNTLLKYRQESHKPKNVRLTPARLKEDFPFLKKTDSLAL |
| | ANAQLNLERAFRNYYKGHAGYPKLKTKRCIWQSYTTNNQHHTIYFQDGKLKLPKLKTLVSLNKHREVP |
| | GQIKSATISAKNNRIFYVSILCKEKVVPLPLTKRSVRLNFSKTCLVEASDSELSFPDFSQAEIEGKLQKAER |
| | KLAVRGKAARNRHISLSQAKNYQKQKEKVRNLYTHYYERKKTYLNELSMQIIRTYDEIYVETNNKRVN |
| | ATGPFTSSDWFHFIQKLKYKAVWYGKTVYLNEENRSKIG |
| 174 | MLRMKAYRFRIYPTEEQRVFLIKTFGCVRFTYNTLLKSGSNMQERLSPAKLKKDFPFLKEVDSLALANA |
| | QRHLDRAFKNYYQGRASYPKLKSKRSRWQSYTTNNQQHTVYIQDGMLKVPKLKSLIPLELHREIKGNIK |
| | TATISAEDSKEFYVSLLCEEDIPIIKKTNKTIKIHFSRERLIEPEMCLEHYYLDILKTEEIIRKAEKRLGVRKH |
| | AALKQHKKLSQAQNYQKQKQRVNRLYVHRQNQKNALFDKLSIHLVREYDRIYIQNLPSQEESEKIFYST |
| | DWQRFLTKLSYKAEWYGKEIILDE |
| 175 | MKKEDLVKVLKGYKFRIYPNEKQIQYFIQTFGCVRFTYNHLLHARQKALQAGDYQTQVSPASLKRDYPF |
| | LKKTDSLALANAQRNLDRAFKNYFSKRAGYPKLKTKKNNWQSYTTNNQKHTIYFVGNQLKLPKLKSL |
| | VTVNLHRKVAGEIKSATVSAQNNQMFFVSLLCLEEINPLPKTGTTIGVAYCPENLVQMSAVNRLPVYKQ |
| | ETLQYQLDKAIKRLEVRAKAAKRRKVLLEQAKNYQKQKSKVQKLYMAKNDQKKNYIDQLTYRLVHD |
| | YDCICLEKQPEFTENTKFSETDWQHFLRKIQYKARWYDKQLVFVDSIEKENETKCFTIEQVGKKLINQ |
| 176 | MQAYRFALDLTPSQERAVWSHAGAGRKAHNWALARVKAVLDQRAAERSYGLADDALTPALRWSLPA |
| | LRKAWNAAKEQVAPWWRECSKEAFNTGLDALARGLKNWSDSRTGKRAGRKVGFPRFKTKHRTTPSV |
| | RFTTGTIRVEPDRKHVVLPRLGRLKLHESARKLARSKRWLRAKARLGRAHARVANLRRDGLHKLTTRL |
| | AREHATVVVEDLNVAGMMANRRLARHVADAGKRLPGTAPAGKTGTVPPQGRAAA |
| 177 | MLKAFKFRIYPTDSQKQWLIQTFGCVRFTYNHLLKARQAYYLETQEIDYTLTPASLKKQYPFLKEVDSL |
| | ALANAQLNLDRAFRNYFKGRASFPKLKNKKSIWQSYTTNNQKGTIYLEETYLKLPKLKEKIRIHAHRPIE |
| | GTIRSATISSRYNEIFYVSLLCEVPPKTMKASNKWIGIAYDPDRLVEMSTPLDITIPKFKQVDQQLLRAKR |
| | KLVIKGRSAQHRRTHVERVKNYQKQKRKIKDLYLKQKFQREDYLEQISGTVIRHYDYLFVESISADCPEG |
| | DFSIQDWHKLLAKLQYKSQWYSKKLVLIDMKEQTNPSTNKKSLELIEIGKQVLFE |
| 178 | MKLGVLKAYKFRIYPNGQQRQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEIPPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 179 | MEKAYKFRMYPNKKQQELINKTFGCCRFVYNKYLAKRIDVYKNDKETFTYKQCSSDLTNLKKELKWL |
| | KEPDKFSLQNALKDLDNAYKKFFKEKTGFPKFKSKKINRFSYKTNFTNGNIMYCGQHIKLPKLGMVKIR |
| | DKQVPQGRILNATISKEPSGRYYVSLCCTDVDIEAFENTNNHIGLDLGIKEFCISSCGEFIENPKYLKKSLN |
| | KLAKLQRELSRKTIGSLNRNKARLKVARLQEHIANQRXKLSILQEMTTFY |
| 180 | MIVVEKAYKFRMYPNKKQQELINKTFGCCRFVYNKYLAKRIEVYKNDKETFTYKQCSSDLTNLKKELK |
| | WLKEPDKFLLQNALKDLDNAYKKFFKEKAGFPKFKSKKINRFSYKTNFTNGNIMYFSQHIKLPKLGMVK |
| | IRDKQVPKGRILNATISKEPSGRYYVSLCCTDVDIEVFENTNNQIGLDLGIKEFCISSCGDFIENPKYLKKSL |
| | NKLAKLQRELSRKTIGSLNRNKARLKVARLQEHIANQRKDFLQKLH |
| 181 | MIAVKKAYKFRLYPNKKQQELINKTFGCCRFVYNKYLAKRIDVYKNNKETFTYKQCSSDLTNLKKELK |
| | WLKEPDKFSLQNALKDLDNAYKKFFKEKAGFPKFKSKKINRFSYKTNFTNGNIMYCGQHIKLPKLGMV |
| | KIRDKQVPQGRILNATISKEPSGRYYVSLCCTDVDIEAFENTNNHIGLDLGIKEFCISSCGEFIENPKYLKKS |
| | LNKLAKLQRELSRKTIGSLNRNKARLKVARLQEHIANQRKDFLQKLSTKLIK |
| 182 | MLKAFKFRMYPTEEQKQQLIRTFGCVRFTYNHLLKERQKSWQQIGVADFSLTPATLKKEYPFLKEVDSL |
| | ALANAQLNLDRAFRNYFKGRASFPKLKTKKSMWQSYTTNNQTHTVYLKNGHLKLPKQKELIKINQHRP |
| | VEGTIRSATISARYNEEFYVALLCDVSSIKKESSAKWIGIAYHPKTLIETSQPIEVTLPKFDQTEEKLQHAQ |
| | RKLSVKVRSAHHRKTRLDKASNYQKQKRKVMDLYLKQKNQREDYLEQLSGKLVKQYDYLFVESFPKE |
| | EAHADFSIHDWHKLITKLRYKSQWYNKKFLLINTDGAEESNSVRKSQVLEQLGRHSVIKE |
| 183 | MKKSSLLNKGYKFRIYPNNEQIAKIEGNFGCARFVYNYYLSQRIGAYNSEGKTIGYLEQQNHLPLLKKH |
| | YPWLKVADSTSLQISIRNLDKAFQNFFNNKAFGFPNFKRKKSLRKCYTVNCVNSNIAVKNGKIKLPKLK |
| | WVEAKVHRKVEGRIISATVIKNSSGRYYVSIITEQKKREISFIEGENIVSLQMKDFINISNEESIYYFKYIKKI |
| | SKLEIKLLRKEKESNNRKKLEKQIGKLYEKVQNKRDDFLHNLSKNIVDENQIIYIQEANVKGEKSSVKDY |
| | QNLKSFSWSKFCKFLEYKSSWYNRSLIYVENNDNYFNLNYGQKLLNTNIPINFGDKINVTLMKKILHPHS |
| | FKYTGIL |
| 184 | MEKAYKFRIYPNKKQQELINKTFGCCRFVYNKYLAKRIDVYKSDKETFTYKQCSSDLTNLKKELKWLK |
| | EPDKFSLQNALKDLDNAYKKFFKEKAGFPKFKSKKINRFSYKTNFTNGNIMYCGQHIKLPKLGMVKVRD |
| | KQIPQGRILNATISKEPSGRYYVSLCCTDVDIEVFENTNNQIGLDLGIKEFCISSCGEFIENPKYLKKSLNKL |
| | AKLQRELSRKTIGSLNRNKARLKVARLQEHIANQRNDFLQKLSTKLIKEXXXXXG |
| 185 | MLKAFKFRIYPTASQKEWFIQNFGCVRFTYNHLLKARQESYARTGAIDYSMTPATLKKKYAFLKSADSL |
| | ALANAQLNLDRAFRNYFNGRASFPKLKNKKSMWQSYTTNNQKGTIYLEDKYLKLPKQKELIQVRLHRP |
| | VEGVIRSATISARYNESFYISLLCEVQIAGVPTTNRWLGVAYDPKKLVETSSPVEVQMPLFRQTRDKMKV |
| | AKRKLVIKSKAAQKRKARLENARNYQKQKRKVMDLYQKQKLQKEDYLERVSGNLIRNYDYLFVEAVP |
| | SELSSADFQLQDWYKLITKLRYKAQWYNKTLLFINVNEQLNEPPEKKSMELEKIGKQVIFE |
| 186 | MIAVEKAYKFRMYPNKKQQELINKTFGCCRFVYNKYLAKRIEVYKNDKETFTYKQCSSDLTNLKKELK |
| | WLKEPDKFSLQNALKDLDNAYKKFFKEKAGFPKFKSKKINRFSYKTNFTNGNIMYCGQHIKLPKLGMV |
| | KIRDKQVPKGRILNATISKEPSGRYYVSLCCTDVDIEVFENTNNQIGLDLGIKEFCISSCGEFIENPKYLKKS |
| | LNKLAKLQRELSRKTIGSLNRNKARLKVARLQEHIANQRNDFLQKLSTKLIKE |
| 187 | MKVLKGYKFRIYPNEKQIQYFIQTFGCVRFTYNHLLHARQKALQAGDYQTQVSPASLKRDYPFLKKTDS |
| | LALANAQRNLDRAFKNYFSKRAGYPKLKTKKNNWQSYTTNNQKHTIYFVGNQLKLPKLKSLVTVNLH |
| | RKVAGEIKSATVSAQNNQMFFVSLLCLEEINPLPKTGTTIGVAYCPENLVQMSAVNRLPVYKQETLQYQ |
| | LDKAIKRLEVRAKAAKRRKVLLEQAKNYQKQKSKVQKLYMAKNDQKKNYIDQLTYRLVHDYDCICLE |
| | KQPEFTENTKFSETDWQHFLRKIQYKARWYDKQLVFVDSIEKENETKCFTIEQVGKKLINQ |
| 188 | MKTIQAYRFALDLTPGQEWAVYAHAGAARVAHNWALARVKAVLDQRAAERTYGVSDDQLTPAVSWS |
| | LPALRKAWNAAKPEVAPWWGEVSKEAFNTGLDALARGLKNWADSRKGKRAGRPVGFPRFKSRRRTTP |
| | SVRFTTGAIRVEPDRKHIVLPRLGRLKLHESARKLARRLEAGTARIMSAAVRRDGGRWHVSFTVEVERA |
| | ERTPDRPGSVIGIDVGIKHLAVLSTGELVPNPRHLATAQDRLRRLGRALSRKSGPDRRTGRRPSKRWQRA |
| | AYAGRWPRSVNPARHQSVRPGPSHRKAGLPTVCSLERTER |
| 189 | MNVLKAYKFRIYPTLEQQQFFIETFGCVRFTYNVLLKNREHQELREYPEEALTPAKLKQDYPFLKKTDSL |
| | ALANAQRNLERAFRNYYAGRCSHPKLKTKKAMWQSYTTNNQQGTIRIENSQLKLPKIKSLVPLHQHREI |
| | KGTIKSATISAKNLEEFYVSLLCEEQVRHLPKTKQKLTIHYSPGQLLTTDQQLDLTAFDQEQLKQKIAKEE |
| | RRLEVRGISARRRLVKLKDAKNYQKQKKRVLALHRHKRARQEAYMDELSLLLVKEFDTIHILATPPQST |
| | GNFSYSDWQKFLQKLTYKAHWYGKTLHHEATAKQG |
| 190 | MKVLKAYKFRIYPTSEQRQFFIETFGCVRFTYNSLLKNREYRDLRDDPGELLTPAKLKQKHPFLKKTDSL |
| | ALANAQRNLDRAFRNYYAGRCSHPKLKTKKAMWQSYTTNNQQGTIRIETGRLKLPKIKTLIPLLLHREIK |
| | GEIKSATISAKNLEEFYVSLLCEEQVAHLPKTSRTISIRFCPQQLVLADAPLSGLGFCQKELSEKLLKEERR |
| | LAIRALSARRRLVKLKEAKNYQKQKNRVMDLQRHKRARQKAYMDELSLTLVKDFDEIIICFEPKQSAVA |
| | FNWSDWQKFLQKLKYKARWYGKTVNLQTLSKNA |
| 191 | MLLTLRQQESGKTVEERTSARLQKQKMTPAKLKKDYPFLKATDSLALANAQRNLEKAFQNYYRGRAS |
| | YPKLKSKKSAWQSYTTNNQGHTIYLAEDGLKLPKLKSKVLVHQHRSVAGKIRSATISAKNRQEFYVSLL |
| | CEEDIPALPKTGSEIEIAYDPTGLVVTNKPIVGIPTFCQTQVLEKLKKAQRRLSCRAKSAQRRNAKLEQAK |
| | NYQKQKSQVQQLYIHKLKQKEDFTEQLSIALLRQFDCIIITKPPELRENKESKAAKTVKKSKHTTVFPSFE |
| | DNFTLSDWNRLLLKLKYKAEWYEKELVFICPTNGK |
| 192 | MDQRAAERSYGIPQEHLTPTIGWSLPALRRWWNAVKGEVAPWWRDYSKEAYNTGLDALARALKNWA |
| | ESRSGRRAGGRAVGFPRFKSRRRSIPSVRFTTGTIRVEPDRRHVVLPRLGRLRLHESARKLARRLQAGTA |
| | RILSATVRRHGHRWYVSFTCHVERAHRTPARPDATVGVDLGVKHERTFTCTTCSLVLDRDVNAARNLA |
| | ALAATVAGSGPETLNGRGADHKTPPTGPVAVKRPPGTATADKTGTVPPQGGTSDHELIPAS |
| 193 | MAGAKSYPVVSVPGVGHRDRLSVVQAYRFALDLSPAQERAVLGHAGAARTAYNWGLERVKAVLNQR |
| | AAERSYGIGDDQLTPTIGWSLPALRRSWNAAKDEVAPWWRDYSKEAYNTGLDALARALKNWADSRSG |
| | RRASRPVGCPRFRSRRRSAPSVRFTTGAIRVEPDRKHVVLPRLGRLRLHESARKLARRLEAGAARFLSAT |
| | VRRDGHRWYVSFTCEVQRAPRTPARPCATVGVDLGVRHLAVLSTGGPVPNPRHLDAALRKLRRLSRGL |
| | SRKVAPDRRTRRDASIRWQRARGQLGRVHARVANLRRTATADKTGTVPPQGRTSNHALTPAS |
| 194 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDTHQLVQQAKYRAEVIEPIQHTKGRLAFLQRRLK |
| | VKARGARKQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 195 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSAWQSYTTNNQNGTVRIEDGYLKLPKLKEKIQICEHRKITGKI |
| | KSVTISAKNNEEFYASILCVETIDKFEKTGKKIRLSFDEHQLVKQAKYRAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 196 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSVFYIKEYDVIEIVEPEDRSCAEDA |
| | LFTSNEWHQLVRLLKYKAQWYGKEIQIINCQNIQKNNW |
| 197 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILVDCKNYQKQKKQYDKLLTHLNNQIKDYLNHLSIFYIKEYDVIEIVEPEDGPCAKDD |
| | LFTSNEWHQLVRLLKYKAQWYGKEIQIINCQNI |
| 198 | MLKAFKFRMYPTEEQKQQLIRTFGCVRFTYNHLLKERQKSWQQTGVVDFSLTPATLKKEYPFLKEVDSL |
| | ALANAQLNLDRAFRNYFKGRASFPKLKTKKSMWQTYTTNNQTHTVYLKNGHLKLPKQKELIKISQHRP |
| | VEGTIRSATISARYNEEFYVALLCDVSSIKKESSAKWIGIAYHPKTLIETSHPIEVTLPKFDQTEEKLQHAQ |
| | RKLSVKVRSAHHRKTRLDQASNYQKQKRKVMDLYLKQKNQREDYLEQLSGKLVKQYDYLFVESFPKE |
| | DAHADFSIHDWHKLITKLRYKSQWYNKKFLLINTDGAEESNFVRKSQVLEQLGRHSVIKE |
| 199 | MSVLKAYKFKIYPNEAQKEFFVKTFGCVRFTYNHLLIARSQTDGKKMTPASLKKEYPFLKETDSLALAN |
| | AQRNLETAFRRYYTGKSDYPKFKNKSNIWQSYTTNNQGQTICLTDGLLKLPKLKTRIAVNEHREIKGQIK |
| | SATISAKNNEEFYVSILCLESIEALPKTTLEIQLRYSPEELLDNLSGLTTLNFDQAAILCKMAKMNRRLKLR |
| | GKIARKKKVPLAYAKNYQKQKVKLSRLQGHQKEKKEDYFNQLSYTLIRDFDRITVDKAKLSDRSDDET |
| | VNFTKADWQTFLRKLQYKADWYGKEIIYQ |
| 200 | MKELKGYRFRIYPDEIQKKFFVETFGCVRFTYNHLLMNKQEPGIDKMTPAQLKQAHPFLKEVDSLALAN |
| | AQRNLERAFRNYHNGRAGYPKLKSKKNSWQSYTTNNQKGTIHLSEGYLKLPKLKERVALNQHREVKG |
| | EIKSATISAKNNQEFYVSLLCLEEIPPLKKTGEVVHLNFDEEHLVQLDRKLILPKFCQEKLEQKIEQAERRL |
| | SCRKKAARRKKIQLQSAQNYQKQKRIVEQLQQEKANQMKNHLEQVSFLLVNHFDKINIQSQSSNLEAPN |
| | AIPNFQLKDWKQFVSKLRYKTQWYQKELVKQK |
| 201 | MKAIQAYRYALDLTPAQERAALAHAGAARVAHNWALARVAAVMNQRAAERTYGVADADLTPAIGWS |
| | LPALRKAWNAPKDEAAPWWRECSKEAFNTGLDALARALKNWSDSRTGKRAGRPVGFPRFKSRRRSVP |
| | SVRFTTGPIRVEPDRKHVVLPRLGRLKLHGFGELRRQLAYKTQWNGGRLIVADRWYPSSKTCSGCGAV |
| | KTKLALSERTYTCTTCGMVLDRDLNAARNLAALATGVDTAGSGPVTGRGADRKTRPGGQVATKRQPG |
| | TAIADQTGTVPPQGRTTDHVLARAH |
| 202 | MVLKAYKFRIYPTNEQKEFLIQTFGCVRFTYNTLLKHHQQNGGGKSKKLTPASLKKEFLFLKVTDSLAL |
| | ANAQQNLKRAFQNYYQGRSGYPKLKLKKSVWQSYTTNNQKQTIWLKDDLLKVPKLKQPIAVHCHRPV |
| | TGQIKSATIMAKNGQQFFVSLLCEEQITPLPKTNVTTTLHFSPDQLVSGSDLVFFRTLCQKNVENKLTKA |
| | KRKLEIKAKSAQQRGVKLSAAQNYQKQKVKVQQLYHHKQQQKKAWMDELSLHLIKKYDFLYIKVPHN |
| | IQEGVFTLTDWQHFLVKLQYKATWYDKKVIFAAEKVI |
| 203 | MKSIRTKLKLNNKQKTLMAQHAGYSRWVYNWGLSLWNAAYRDGYQPNARKLREVFTNHTKPLYPW |
| | MKSLSSKVYQYALINLGEAFKRFFQGLGKYPRFKKKGKHDSFTIDNFGKPIELNGWSHKLPFIGMVKTY |
| | EPIEATTQKITISRQADDWYLSLAFEFTPTSTEKITDVVGVDLGVKTLATLSTGEVFNSVKPYRKAQNKLA |
| | KLQRQVSRKVKHSRNWYKAVIKLAKQHRRVANIRKDALHKLTTYLAKVRLVPVRSL |
| 204 | MKSIRTKLKLNNKQKTLMAQHAGYSRWCYNWGLSLWNAAYKDGFKPNARKLRDVFTNHTKPLYPW |
| | MKNLSSKVYQYAFINLSEAFKRFFKGLGKYPRFKKKGRSDSFTIDNCGKPIELNGWNHKLPFIGMVKTY |
| | EPIEATTQKITISRQASDWYLSCSYEFTPTATSKTTEVVGVDLGIKTLATLSTGEIFKSVKPYRQAQNRLA |
| | KLQRQLSRKVKHSNNWYKVVIKLAKQHRQVANIRKDALDKLTTYLAKVRLVPVRSL |
| 205 | MVVQAYRFALDPTPAQDRDLHRHAGAGRFAFNWALAAVRANLDQRAAERTYGLDGEQLTPALGWSL |
| | PALRRAWNTAKPQVVPWWGQCSKEAFNTGLDGLARALGNWSASRSGRRAGARVGFPRFRSRRRVTPS |
| | VRFTTGTIRVEDTRHHVTLPRLGRIRTHESTRKLARRLHAGTARIMSATVRHTGGRWHVSFTVQITRTVC |
| | TPAYSQQTVGVDVGIANLAVLSTGQIVPNPAIWPPLPGGCAPQPGPCPDGKDQTGAPAGSRPGAGSRPKP |
| | AWPAHTPGSRTCAPTACTNSPRPWPAPTARSWSRTSTSPG |
| 206 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTNNEWHQLVRLLKYKAQWYGKEIQIINCQNI |
| 207 | MSVLKAYRFKIYPDEAQKQFFVATFGCVRFTYNHLLVASQQKKEQKLTPAKLKKEYPFLKETDSLALAN |
| | AQRNLEKAFRRYFTGKSDFPKFKHKSNPWQSYTTNNQGHTIYLKEGQLKLPKLKSLVKVNYHREITGQI |
| | KSATISAKNNTDFYVSILCVEEIPSLPQTSQSITIAYSPSELLEGSQSLLQITFNQDSLVTKIDKVQKKLKIRA |
| | KVARKNRIPLAEAKNYQKLKERLARLQVSQKEKKEDFFDQLSYYLVCHFDQIMVDATIIENNQEACTVV |
| | FTKADWHCFYKKLVYKSNWYGKKLIDLD |
| 208 | MKKLKGYRFRIYPEEDQRQFFIETFGCVRFTYNHLLMAKKDKTVESLTPAQLKKDYPFLKKTDSLALAN |
| | AQRNLDRAFSNYYRGRAGYPKLKNKKAIWQSYTTNNQKNTIQLLNGTLKLPKLKTAVKVEQHRVVHG |
| | LIKSATISAKNNTEFYVSLLCLEEIQPLEKTGEKTIVMFHPTTFIQTDANITLPTLDLNPLNQKIEKEQLKLV |
| | RRKKVARTRGVALSDSKNYQKQKQRVEQLVLTKSNKKINFFDQLTWILVQQFDKIAISTPAPEDFCEEGL |
| | YSPSDWQQFLIKIHYKIDWYQKELHQQQK |
| 209 | MISEGRQVPIKILKAYKFALYPDEAQKQFFIQTFGCVRFTYNTLLTLRQTNYQDNSETFTNPASGRLKTQ |
| | KLTPAKLKKEYSFLKATDSLALANAQRNLEKAFQNYYRGHASYPKLKSKKSAWQSYTTNNQGHTIYLE |
| | KDGLKLPKLKSKVLLHQHRNVTGKIRSATISAKNRQEFYVSLLCEEDSTALPKTGSKIEITYNGITLIEPSV |
| | AVRGIPTLCQVQLLAQLKKAQRRLAIRAKSAQRRNVKLEQAKNYQKQKLRLQQLYIRKMKQKEDFTEQ |
| | LSIALVRQFDCIVVTMPAAGDDETKNKGNKALKTQKNNQNTPVLQNIEEKFTLSDWNRLLLKLKYKAD |
| | WYEKELVFCSKQKAK |
| 210 | MSVLKGYKFRIYPDEKQKKFFIETFGCVRFTYNHLLMARQTGAARNTTMTPASLKKEYPFLKKTDSLAL |
| | ANAQRNLDRAFRNYFSGRAGYPKLKTKKSTWQSYTTNNQQHTVYLEGEYLKVPKLKSLVPVHLHREIR |
| | GKIKSVTISAKRNREFYASILCVEEVEELPKTNDLVGISYCPENLIQISAQRELPQIDQSHLIKQLGKEQKKL |
| | QLRAKVAKKRKVRLINAKNYQKQKERVLKLRTTKLDQKRNFIDQLTISLVRDFDYLFIESKPKFQNESGE |
| | FSEADWQQFIQRIQYKGRWYGKEVRYIEVKELKNEKCKEIERLGRAQLT |
| 211 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILVDCKNYQKQKKQYDKLLTHLNNQIKDYLNHLSIFYIKEYDVIEIVEPEDRSCAKDD |
| | LFTSNEWHQLVRLLKYKAQWYGKEIQIINCQNI |
| 212 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYALILCVETIDKFEKTGKKIRLSFDAHQLVQQAKYRAEVIEPIQHTKGRLAFLQRRLK |
| | VKARVARKQNRILVDCKNYQKQKKQYDKLLTHLNNQIKDYLNHLSIFYIKEYDVIEIVEPEDRSCAKDD |
| | LFTSNEWHQLVRLLKYKAQWYGKEIQIINCQNI |
| 213 | MISEGRQVPIKILKAYKFALYPDEAQKKFFIQTFGCVRFTYNTLLTLRQTNYQDNSETFTNPASGRLKTQ |
| | KLTPAKLKKEYSFLKATDSLALANAQRNLEKAFQNYYRGHASYPKLKSKKSAWQSYTTNNQGHTIYLE |
| | KDGLKLPKLKSKVLLHQHRNVTGKIRSATISAKNWQEFYVSLLCEEDSTALPKTGSKIEITYNGTTLIEPS |
| | VAVRGIPTLCQVQLLAQLKKAQRRLAIRAKSAQRRNVKLEQAKNYQKQKLRLQQLYIRKMKQKEDFTE |
| | QLSIALVRQFDCIVVTMPAAGDDETKNKGNKALKTQKNNQNTPVLQNIEEKFTLSDWNRLLLKLKYKA |
| | DWYEKELVFCSKQKAN |
| 214 | MGKNQRKVLKAYKFRIYPTKAQQKFLIQTFGCVRFTYNTLLKQRQFNTIEASKKLTPAALKKEFPFLKLT |
| | DSLALANAQRNLARAFQNYYQGRSGHPKMKLKKSTWQSYTTNNQQQTIWLKDNLLKVPKLKQPIAVV |
| | CHRKVVGKIKSATITAKNLQQFYVSLLCEEEVGHLPKTKTEIELRFAPNQLVVGNQLKFCRQLCVNDLET |
| | KLKKAKRKLEIKAKSAQQRKVRLAEAKNYQKQKLKVQKLYHHKQQQKKAWIDELTMHLIKNYDFLY |
| | VEVPKNGIEGSFTLADWQSFLVKLQYKANWYGKKVIFLTAAKTVRKIS |
| 215 | MDLTPRQERAVLAHAGAARVAHNWALARVKAVMDQRAAERTYGIDEVDLTPTQGWSLPALRRAWN |
| | QAKADVAPWWAECSKEAFNTGLDALARGLKNWSDSRTGKRAGRRVGFPRFKSRRRSTPSVRFTTGAIR |
| | VEPDRRHVVLPRLRRLKLHESARKLARRLEAGTARVVSATVRRDGGRWYVSFTCAVQRVQGVPACPD |
| | ATVGVDLGVSHLAVLSTGEVEPNPRHLDVAARRLRRLARRGPLAGSARTGAPVGVGRCGGSARPARSG |
| | ACPGCSSAP |
| 216 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDAHQLVQQAKYRAEVIEPIQHTKGRLAFLQRRLK |
| | VKARVARKQNRILVDCKNYQKQKKQYDKLLTHLNNQIKDYLNHLSIFYIKEYDVIEIVEPEDRPCAKDD |
| | LFTSNEWHQLVRLLKYKAQWYGKEIQIINCQNIKKITGRGARVRLGNN |
| 217 | MKNLKGYRFRIYPNEAQKRFFIETFGCVRFIYNYFLKLDTAERTSEEVITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISAKNNEEFYASILCVETIDKFEKTGKKIRLSFDEHQLVKQAKYRAEVIEPIQQTKGRLEFLQRKLK |
| | VKARVARKQNRVLADCKNYQKQKKQYDKLLTHLNNQIKDYLNHLSIFYIKEYDVIEIVEPEDRSCAKDA |
| | LFTSNEWHQLTRLLKYKAQWYGKEIQIINCQNI |
| 218 | MKNLKGYRFRIYPNEAQKRFFIETFGCVRFIYNYFLKLDTAERTSEEIITPASLKRDYPFLKKTDSLALAN |
| | AKRNLDRAFQNYYQQRSGYPKLKNKSSVWQSYTTNNQNGTVRIEDGYLKLPKLKEKIQICEHRKITGKI |
| | KSVTISANNNEEFYASILCVETIDKFEKTGKKIRLSFDAHQLVQQAKYRAEVIEPIQHTKGRLAFLQRRLK |
| | VKARVARKQNRILADCKNYQKQKKQYDKLLTHLNNQIKDYLNHLSIFYIKEYDVIEIVEPEDRSCAEDA |
| | LFTSNEWHQLVRLLKYKAQWYGKEIQIINCQNI |
| 219 | MKNLKGYRFRIYPNEAQKRFFIETFGCVRFIYNYFLKLDTAERTSEEVITPASLKRDYPFLKKTDSLALAN |
| | AKRNLDRAFQNYYQQRSGYPKLKNKSSAWQSYTTNNQNGTVRIEDGYLKLPKLKEKIQICEHRKITGEI |
| | KSVTISAKNNEEFYASILCVETIDKFEKTGKKIRLSFDEHQLVKQAKYRAEVIEPIQQTKGRIEFLQRKLKV |
| | KARVARKQNRVLADCKNYQKQKKQYDKLLTHLNNQIKDYLNHLSIFYIKEYDVIEIVEPEDRSCAKDAL |
| | FTSNEWHQLTRLLKYKAQWYGKEIQIINCQNI |
| 220 | MKVLKGYRFRIYPDEEQLTFFRQTFGCVRFTYNQLLMARKNTANSEESMKLTPAALKKDYPFLKKTDSL |
| | ALANAQRNLERAYANFFQGRASYPKLKNKKSTWQSYTTNNQKHTIYFVDEKLKLPKLKSLIQVHQHREI |
| | KGLIRSATISAKNNEEFYVSLLCLEEVTSLPKTKKAIGISYCPKHLLHVSKPLDHLETIEEQMQEDRLIKAK |
| | RKLFLRAKIAKKHKVKLKDAKNYQKQKQKVHKLIQEKACRKKDFIDQLTFSLVKEFDYIFVEKQPSTAD |
| | SEETSLFTSSDWYLFMQKLTYKTQWYGKKYLAIEKPANTENSGQMIEELGKQRLGL |
| 221 | MKVLKGYRFRIYPDEEQLTFFRQTFGCVRFTYNQLLMARKNTANSEESMKLTPAALKKDYPFLKKTDSL |
| | ALANAQRNLERAYANFFQGRASYPRLKNKKSTWQSYTTNNQKHTIYFVDEKLKLPKLKSLIQVHQHREI |
| | KGLIRSATISAKNNEEFYVSLLCLEEVTSLPKTKKAIGISYCPKHLLHVSKPLDHLETIEEQMQEDRLIKAK |
| | RKLFLRAKIAKKHKVKLKDAKNYQKQKQKVHKLIQEKACRKKDFIDQLTFSLVKEFDYIFVEKQPSTAD |
| | SEETSLFTSSDWYLFMQKLTYKTQWYGKKYLAIEKPANTENSGQMIEELGKQRLGL |
| 222 | MIAVEKAYKFRVYPNKKQQELINKTFGCCRFVYNKYLAKRIDVYKNNKETFTYKQCSSDLTNLKKELK |
| | WLKEPDKFSLQNALKDLDNAYKKFFKEKAGFPKFKSKKINRFSYKTNFTNGNIMYCGQHIKLPKLGMV |
| | KVRDKQVPKGRILNATISKEPSGRYYVSLCCTDVDIEAFENTNNHIGLDLGIKEFCISSCGEFIENPKYLKK |
| | SLNKLVKLQSELSRKTIGSLNRNKARLKVARLQEHIANQRNDFLQKLSTKLIKENDI |
| 223 | MKQKKRFFIETFGCVRFIYNYFLKLDTAERTSEEIITPASLKRDYPFLKKTDSLALANAKRNLDRAFQNY |
| | YQQRSGYPKLKNKSSVWQSYTTNNQNGTVRIEDGYLKLPKLKEKIQICEHRKITGKIKSVTISAKNNEEF |
| | YASILCVETIDKFEKTGKKIRLSFDAHQLVQQAKYRAEVIEPIQHTKGRLAFLQRRLKVKARVARKQNRI |
| | LADCKNYQKQKKQYDKLLTHLNNQIKDYLNHLSIFYIKEYDVIEIVEPEDRSCAEDALFTSNEWHQLVRL |
| | LKYKAQWYGKEIQIINCQNI |
| 224 | MKNLKGYRFRIYPNEAQKRFFIETFGCVRFIYNYFLKLDTAERTSEEVITPASLKRDYPFLKKTDSLALAN |
| | AKRNLDRAFQNYYQQRSGYPKLKNKSSAWQSYTTNNQNGTVRIEDGYLKLPKLKEKIQICEHRKITGKI |
| | KSVTISAKNNEEFYASILCVETIDKFEKTGKKIRLSFDAHQLVKQAKYRAEVIEPIQQTKGRLEFLQRKLK |
| | VKARVARKQNRVLADCKNYQKQKKQYDKLLTHLNNQIKDYLNHLSIFYIKEYDVIEIVEPEDRSCAKDA |
| | LFTSNEWHQLVRLLKYKAQWYGKEIQIINCKNI |
| 225 | MLKAYKFRIYPTNEQKEFLIQTFGCVRFTYNTLLKHHQQNGGGKSKKLTPASLKKEFLFLKVTDSLALA |
| | NAQQNLKRAFQNYYQGRSGYPKLKLKKSVWQSYTTNNQKQTIWLKDDLLKVPKLKQPIAVHCHRPVT |
| | GQIKSATIMAKNGQQFFVSLLCEEQITPLPKTNVTTTLHFSPDQLVSGSDLVFFRTLCQKNVENKLTKAK |
| | RKLEIKAKSAQQRGVKLSAAQNYQKQKVKVQQLYHHKQQQKKAWMDELSLHLIKKYDFLYIKVPHNI |
| | QEGVFTLTDWQHFLVKLQYKATWYDKKVIFAAEKVI |
| 226 | MKVLKAYKFRIYPNEEQIQYFIQTFGCVRFTYNQLLYTRKKALQEGDYETRLTPAQLKKDYPFLKQTDS |
| | LALANAQRNLDRAFKNYFSKRAGYPKWKSKKSSWQSYTTNNQKHTIYFIGEELKLPKLKSLIKVNLHRE |
| | ILGEIKSATISAKNNQLFFVSILCLENVVSLPKTGKSIGIAYCPENLVQMSSTNVFLNRKSNSYYQLKTAKK |
| | KLELRARLAKKRKVLLSQAKNYQKQKRKVQKLYMKIDNQKNDYINQLTYYLVKNYDHIYLEKYPKFS |
| | ENVQFSETDWQHFLRKIQYKVSWYNKQLVFIAPNTKEIEEKCFAIEQLGRQLTTS |
| 227 | MMEVTRVIVIQLKPTKEQKIILKHLTYSASKLWNIANYNIKQGNIKPKELKPTLKENFWYKNLHSQSAQA |
| | VLEKLQIAWENCYKKHTKEPRFQPKDGHFPVRWKKQGLQINNGQIRLSLSKQTKQYLKNMHSIKSDYL |
| | WISLPKNLSLNGVQEVEIKPPSSKKLHYLIIKERNYVRDYIHKVSTFIVREALSKDVKTIAIGKLSKNITKID |
| | IGRQNNEKLHKIPFGKLCNMIEYKAKEVGINVIYVNEGYTSQTCSICGDVNKTNRKYRGLYICKCGNVIN |
| | ADVNGGINILKRVSPNLTLGRSRGNLNIPTRVRMYNTL |
| 228 | MRADTVYSSLRKSRYESWNVLPSILPYSGNEANYQRRQAFLKKQKKLPNYSQQCRHFKHSDNFKAIGT |
| | GKGQAVLKKLDEAWSSFWTLKRLQSEEGRLPPNIRRVRMPSYLKDRDSKQTVVRGFYVRNDCYRLDR |
| | KRSTITIIGKNLRLRYAADRVREGKKGRLDVMYDRLKDAWYAFIPVDTAPAKQAVVSGQPEKVGSIDLG |
| | ICNLVAFYAENEQPVIYSGRAVLSDYVYRTKKIAELQSRLPQKQQHTSRKIGLSYRKRTRRFKHATRAML |
| | KDLFERMKQIGITKVAVGDLNGIRDGNNLGAHTNQKLHNFWSHLQTIEWIRHMCEDYSMEFVQVSEKG |
| | TSKTCCVCAVKDIMAGYIEACTSAKKTG |
| 229 | MKQQVSFKFRLKPDGQQERQMRRFAGACRFVFNRALALQNENHEAGKKYIPYTKMASWLVEWKKDT |
| | ETEWLKDSPSQPLQQSLKDLERAYKNFFQNRAAFPRFKKRGQNDAFRYPQGVKLDQENSRIFLPKLGW |
| | MRYRNSRQVTGIVKNVTVSQSCGKWYISIQTEREVSTPVHPSASMVGLDAGVAKLATLSDGTVFEPVNS |
| | FQKNQKKLARLQRQLSRKVKFSNNWQKQKRKIQRLHS |
| 230 | MSKNLYNLSTYRYRQHFFQTGQKLSFNDLYHQVSKTSAYYALPNTKVAKQIIRRIDQSWKGYFQAHKD |
| | WSRVDNYLHTVSRRVIDWCLLNSIGTLVIGKNDHWKQSINIGKKNNQQFVSIPHARLIEMLCYKGELMGI |
| | KVVVTEESYTSQSSFLDFDTLPSYGEKKPKFSGKRIKRGLYKTSTGKLINADVNGSYNCIRKHLQQEKVK |
| | SNAFHSHDLMALPFMPVTYDPLRTHNLNFLQIV |
| 231 | MHESFALVNASKLWNVARWTAGRVWDACGQIPDDGVLKSYLKGSGRYVDLHSQSSQRVLEELAEAFT |
| | GWYGHRNNGNQKAXXRSTVTFKQAGFKHDTENQRIRLSKGRNLKDHRSDFVLCEYDVIGPRGTTVEN |
| | VQQVRAVHEHGIWRLHIVCNVEIDVPDAPGNGVAGIDLGICNVAAVVCNVEIDVPDAPGNGVAGIDLGI |
| | CNVAAVSFGDESLLYPGGALKEDDYYFAKKRAECDECSSREARRLDQKRTDRRTHFLHALSKHIVQQC |
| | VERGVGTIVVGDLGG |
| 232 | MPRRRDVDTEPVVHRTARIGLRLTRAQRQRCFGLLRCAGDVWACLLEINWWRRHRGDPPVAGYQQLC |
| | RLLAESGPGIFGELDSAGARSVLRRYSDAWFSAAARRKAGALEVRYPRRRRASMPIRWYNGTFTLTGRA |
| | LRLPTARGCPPLMVRLDRHLPYPPGTVRSVTLLFADGRLCIDVTAELPVTTYPAERAPDPQRVAGVDLGI |
| | IHPFAVAGPDGEALLVSGRAIRAEHRLHLADTEHRQRATADRAPSRGQHGSRRWRKTRRRARLVEGRH |
| | RRRVRQALHEAAKTVIHWAIQQRIGTLTVGDPRGVLNLQAGRRHNLRLRXWQIGRTLQILHDKATLAGI |
| | QLHLVDERAPRRPVPTAGNASPNPPAGPCPARIASSPGIVISPRRSPSPPAPRAAQPPPSASRRLGWLRTVE |
| | PDDTSPESPRHGVTPAADHRRPAGPLAGGGPPLSQGSRSPTPVSEDPQHHRTQPGRRSWTHRTSVTAWT |
| | SSSCCPSWSCFGFRWGTPISGAIRAGSP |
| 233 | MLKSFKTEIDPTLEQKQKIHQTIGTCRFVYNFYLSHNKEVYEAEKKFVSGMDFQKWLNNVYLKEHPEFS |
| | WIKDVSSKSVKQSIMNADKAFRSFFKHQTSFPKFKKKGTSDVKMYFVKTDAKAVIYCERHRIKIPTLGW |
| | VRLKEKGYLPTTKTGMVIKSGTVSMKAGRYYASVVVEIDDSPSVKNNENGGIGIDLGLKDLAIVSDGKA |
| | YKNINKSAKIKKLEKRLRREQRSLSRKLENTKKGESTQKNIQKQKLKVQKLHQ |
| 234 | MHYAYRYRLNPTEAKQETLDCHRDTCRQLYNHALTEFEQIPDSAGTLNQRVRQVRDQLTDLKHWWDE |
| | LTDLYSTVAQAAVVRIEQSLKALSGLKQNGYNVGSLNWKAPSEFRSFTYVQSGFEFDNKNGQTVLSLSK |
| | LAEIPITVHRQIPDGITVKEVSLKKERTGEWYGSFAVEGKEEPEKPENPDRCVGVDAGILTYAHDTDGCA |
| | VGSVELQDERERLTRDQRSLSRKQQGLNDWEKQRLRVAEFHQRVRRKRHDFLQKLSKYYAPEYKLVA |
| | GSC |
| 235 | MIKKQAFKFLLEPNKGQLSDFLAFAGSCRFVYNKGLALLNENYRSGKKFIGYNQLASELVEWKNEESLS |
| | WLKEAPSQCLQQSLRDLDKAFRNFFTGKSQYPKFKKKGRHDSFRTPSQRVRVDQEKKLVSLPKVGWVK |
| | YRKSREIIAELKNVTISMKQGKWYISFNTEHTVPDPIHPSDIKTKIVLNNVNSVHLSSGIGGDNTSQAEEK |
| | KKLIRLNKRLARRKKHSKNWLKTKGKIDRVKSKAARLRLDNIHKATTAICKSHAVIEVVNLMGSVSDK |
| | NDNTLSMRYEFVRQLIYKQEWLGGEVIRRESKPL |
| 236 | MHYAYRYRLHPTESQRETLDYYRDTCRQLYNHALTEFEQIPQSAGTLNQRVRQVRDQLPALKNWWDD |
| | LTDLYSTVSQAAVMRIEDSVKALSQLKQNGYNVGSLNWKAPREFRSFTYVQSGFEFDRKNGQTVLSLS |
| | KLADIPLTRHRDIPDSVTVKSVTVKKERTGEWYASFAVDGKDEPTKPDNPDRCVGIDVGILKYAQDTDG |
| | RAVGFPDLQDERERLRREQRALSRTQQGSNNWHKQRQTVAECYQALRRKRHD |
| 237 | MANYQTMQIWVKKNHRMHGYFKEMCQHAKNMHNTTNFYIRQVFTAFTQEKAFQPLQEEVLDTIQKH |
| | MPIINANQFVVYQKKVVKEHSKPARERKEIKCHLFKEPSRENPYVDYNFLDALFKSMAQEDYRSLPTQS |
| | SQGVMKTVFQNWKAFYGSLREYKTNPSKFKARPKIPGYRRKKEKEVLFSNQDCVIKENKFLKFPKTKER |
| | LNIGKLGFTEGRLKQVRLIPKYGHYMVELIFQMPSEQEMKASKKRYMSVDLGMDNLATIVTNTGRKPVI |
| | VKGKNIKSINQRYNQLKAHYHGILRQGKNTNEGLFTSKRLEKINYKRFNQIKDLFHKASYRIEKIALEEDI |
| | DTIIIGQNKAWKQHAKMGKRNNQSFTTIPHRLLMQMIKYKAQRHGIKVIVTEETGRVSRMSMRRHLPQN |
| | LPYNQYLSMLFSWIIPHFLNVMKLVMIINECYFISPSTCFSQSSSTPQAISGSPFNDFIVPTYTLFVDLPISI |
| 238 | MKRERGHKARLYPDSDQLSALEDQGHASRAMWNLLHDWWTMASENRRVQLKEADQAIRQARKDID |
| | WLSDLPAQAAQAVLKTYFRAWGNYWEGRAKAPTFKARFRSRMAIDVPQGRDLGIRRITRRWGVVKVP |
| | KVGMIRFRWTKDLPVGKHTDVKNKVTGARLVREANGWHVVFRVRTETKELAPQLGPGVGIDRGVAKP |
| | LALSDGSFREHDPWLSRLSRNVCADWRRQRHVRSTLASAVREHRTVSSGPMTRSQACAREPSAAPWTG |
| | STRPPPDLPRPSA |
| 239 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLARRKKYSKNWLKTKGKIDRVRSKAARIRLDNIHKATTAICKNHAVVEVVNLMDSVSAK |
| | NDNTPSTRYEFVRQLIYKQEWLGGEIIRRESKLL |
| 240 | MIKKQVFKFLLEPNKGQLSDFLAFAGSCRFVYNKGLALLNENYRSGKKFIGYNQLASELVEWKNEESLS |
| | WLKEAPSQCLQQSLRDLDKAFRNFFTGKSQYPKFKKKGRHDSFRTPSQRVRVDQEKKLVSLPKVGWVK |
| | YRKSREIIAELKNVTISMKQGKWYISFNTEHTVPDPIHPSDIKTKIVLNNVNSVHLSSGIGGDNTSQAEEK |
| | KKLIRLNKRLARRKKHSKNWLKTKGKIDRVKSKAARLRLDNIHKATTAICKSHAVIEVVNLMGSVSDK |
| | NDNTLSMRYEFVRQLIYKQEWLGGEVIRRESKPL |
| 241 | MIKKQAFKFLLEPNKGQLSDFLAFAGSCRFVYNKGLALLNENYRSGKKIIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKSTIVLNNVGSVHLSSGVGGDNTYQAEEK |
| | KKLIRLNKILTRRKKHSKNWLKTKGKIDRVKSKAARIRLDNIHKATTAICKNHALVEVVNFMDSVSDKN |
| | DNTLSMRYEFVRQLIYKQEWLGGEVIRRESKPL |
| 242 | MPRPSGRGGIGLLPNRAGTAGSRVRVIPSPPGTVVSKPPCIGGFQLPYLTGRLRVVTAGVQASGVAGYAR |
| | YTYRLRVSSTASGLLLAEWDRCRWIWNECVARAKKAHRDGEKCGPAALDRMLTETRRMTPWLREGSS |
| | VPQQQLIRDYGKARGKALKDIKDRLPNHRRAEMPRWKKKREARPSLNYTRRGFRLADGRLHLAGGITV |
| | PVVWSRDLPAVPSSVRVYQDSLGHWCASFVVPAQVEPLPSTGRVIGVDLGVRETATTTSDAYDLPHAEY |
| | GRKAAAGLARYQRMMARRKPVKGQAASRGYQEAGKQVARLHRKVARQRQDTARKWAKAVVRIMM |
| | PWRWRTSARSSSRKRRWPARPLMPRSVRRRQPWSRWAASTDAWCTWFIPRTPRWTAHGAEREPSTHS |
| | RSPNEPMPAPRAEPYPPGIKTPRA |
| 243 | MIKKQAFKFLLEPNKGQLSDFLAFAGSCRYVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGEWYISFNTEQTVPDPIHPSDIKTTIVLNNVNSVHLSSGVGGDNTYQAEEK |
| | KKLIRLNKTLTRRKKYSKNWLKTKAKIDRVRSKAARIRLDNIHKATTAICKNHAVVEVVNLMDSVSAK |
| | NDNTLSMRYEFVRQLIYKQEWLGGEIIRRESKLL |
| 244 | MLACIAAHVLCGHVREYRPLLAKLSAQRFQKLNEVFDGRRLETLDQAMDVLHPAPWFRFQTQTNSVRD |
| | PHALQELEDFDHTNKWPTKQVKHERKIDAFDTRADRAQEVSNLTSTINILKCDAATHANDIVSASALRK |
| | EMSAKDKLMIRRIRKYRLFPTAEQRKKLHQFMGTCRWTYNKGVAHFRKTNVSSAQTLRDLYVTEKSKK |
| | QRVYPEDMEPPPQWAYETPKTFRFNALRKFESGVKSAFSNKINGNISKFKIQFKSRKKDGRYFTFCEDAG |
| | RANIMYKTGESRAMLSISKLKNIPIKAFGQVSSPNPKRTTGDGVDQELQRPSFRKAVASARESQASVPLR |
| | TAKLSNCVKEMHYQTSTYLTKHYDTIILPVFNSSVMVKKSNARNHTFNRLLSGLKHFQFRKLLQAKCEL |
| | MGKSLVVCSEMYSSQTCGRCARLHLKLGSRDMFSCPHCEHVAGRDVNAAFNILRFVCAGSLVVSATHH |
| 245 | MKLSFKFFPELTFLQLDILEELCYHTTKLYNIANYECITEGYKSYYEMEKLHTTNWHKAFLHSHTYQQCL |
| | KLLEQDWKSYFAAAADYKNNPHKYKAMPMPPKYKNVENHKNHIIFTNLAVRFKNNILMFSLAKEVQT |
| | KFGAESLNFEVSDKLQKLMNLDSIQQAKNSYFNKQIAYYTSLEMKKSGSTRFKRTARIRKLQKQRNDCI |
| | QDCLHKASRKLIDLALLHNCSTIVAGDISGIKQESPIKGFVQIPIQRLVEQIKYKVELVGMKVILQNESYTS |
| | GVSAIDLEPVNKEYYNKNRRIARGLFKSNAGILINADINGSLNILRKYKNVVPELVKQARDNGLVDNPIRI |
| | AA |
| 246 | MARRELELFTLPTKEKAFVGYNFLDALFKTIKQKDYYSLPGQINQQVIKNVVQNWDSFFKSLNGYNVNP |
| | QKYKGRPNIPGYLPKGSKKEVVLSNQICQLKGEKYLRFPKTRGKLNIGKLANVSGKFQQVRIIPKYDNFT |
| | VEVIFLMGKKVEISPKKKRILSIDLGVENIATLVSNVEMTPILFKGGKIKSINRWYNKLKSYYYAALRNGR |
| | SSKEGQRYSKRLSKLDSKRHNQIKDFFHKVSFNIVKVAKEHRIDTIIIGKNMDWKQKVALGSKNNQNFV |
| | QIPHAMLVSMIRYKANTEGIAVIETEESYTS |
| 247 | MDVKKGHRLFSYFEELCANGNNLYNLTNFYIRQVYTALKSDKPLQPLQREVLETIYRNIDKMNEKKTIA |
| | YYKKLKKENLLGKEKQKELELFTLPTKEKAFVGYNFLDALFKTIKQKDYYSLPGQINQQVIKNVVQNW |
| | DSFFKSLNDYKENPQKYIGRPSIPRYLPKGSKKEVVLSNQICLLKGEKYLRFPKTRGKLNIGKLANVSGKF |
| | QQVRIIPKYDSFTVEVIFFIGKKVEINPKKKRILSIDLGVENIATLVSNVEMTPILFKGGKIKSINRWYNKLK |
| | SYYYAALRNGRSSKEGQRYSKRLSKLDSKRHNQIKDFFHKVSFNIVKVAKEHRIDTIIIGKNMDWKQKV |
| | ALGSKNNQNFVQIPHAMLVSMIRYKANTEGIAVIETEESYTS |
| 248 | MKQQVSFKFRLKPDGQQERQMRRFAGACRFVFNRALALQNENHEAGKKYIPYTKMASWLVEWKKDT |
| | ETEWLKDSPSQPLQQSLKDLERAYKNFFQNRAAFPRFKKRGQNDAFRYPQGVKLDQENSRIFLPKLGW |
| | MRYRNSRQVTGIVKNVTVSQSCGKWYISIQTEREVSTPVHPSASMVGLDAGVAKLATLSDGTVFEPVNS |
| | FQKNQKKLARLQRQLSRKVKFSNNWQKQKRKIQRLHSRIA |
| 249 | MKQQVSFKFRLKPDGQQERQMRRFAGACRFVFNRALALQNENHEAGKKYIPYTKMASWLVEWKKDT |
| | ETEWLKDSPSQPLQQSLKDLERAYKNFFQNRAAFPRFKKRGQNDAFRYPQGVKLDQENSRIFLPKLGW |
| | MRYRNSRQVTGIVKNVTVSQSCGKWYISIQTEREVSTPVHPSASMVGLDAGVAKLATLSDGTVFEPVNS |
| | FQKNQKKLARLQRQLSRKVKFSNNWQKQKRKI |
| 250 | MLSGMIALRNIEHKRIITRPCERGRRSTVGTKRHFTNRTRLLKRLKIQRAPTAISICRLPLRSNPAITNKPPG |
| | YRKRGDRHPRSTVTWKQNGIKHDDKHGQLRLSKGWNLKDGRSDFILVEYETRPDVTVENIQQVRAVW |
| | TGDRWELHLVCETVIPVEDAPGDNTAGIDLGISNYLAIDYEDGPSELYPGNRLKQDKHYFTRDEYQIEGE |
| | NGPSKRARRARRKLSRRKDHFLHTLSKHIVERCIEEEIGTIAVGDLSDIREDDDGDSRDWGQRGNKKLHG |
| | WEFDRFTRLLEYKAEAYGILVDRVDEENTSKTCSCCGQI |
| 251 | LDALCDYRRYCWNKGLETWQLMYEAHTLNKKDNPSPNERRVRDELVTNKADWQYDLSARCLQLAIK |
| | DLANAWKNFFDKAQPDWGIPSFKSKKSPRQGFKTDRAKIVNGKLRLDRPRSISKDSWHDLSSYEVLKMS |
| | EVKVVSIFKEKGAYYAALTYEEEPISKTKTHQKTAVDVNVGHFNYTDGKINVLPAKLQKLYKRIKHYQR |
| | MLARKREVNGKLATKSNNYYAVRIKLQRDYRKVANIQNDLLQQFTTKLVDNYDQ |
| 252 | MSGDLNQCGFSASKLWNVARYYTQGRWDEDGEIPDDGELKSELKEHERYRDLHSQSSQRVLEELAESF |
| | TSWYKARQRGDEDANPPGYRKHGDNHPRSTVTWKQKGIKHDPKHNHLRLFKGFNHKSEGENGPSRRA |
| | LRACQKLSRRKDHFLHALAKHIVERCIDHEVGRIAIGNLSKIREAENGEARNWGKRGNKKLHGWAFDRF |
| | ATLLEYKAEEHGILVERKSERDTSKTCSCCEQKRDANRVERGLYVCASCGGTMNADVNGAVNIRRKIT |
| | QNPPTEDMSNGRLARPVTYLFNQTSGSFHPREQVGCEL |
| 253 | MYNPREHRNYKFRIYPTKTQAETLTNWLTLCRLCYNAALVDRKNHYLRNKASLTRTKQQTTLKLDKDK |
| | HPQLKEVHSQVLQEVLFRVEKAYQAFFRRVKAGENPGYPRYKGMGQYNSLTYTQFGDGRGAYFKEEK |
| | LALSKIGLLKIKLHREIPGKVKTCIIKRETTGKWYAVLSVEGYPVLYSPNWKKTGLDVGIEKLATLSDGE |
| | QIPNPKPIQKSEKKVKRAQRDLCRKKKGSKNREKARQRLAQIHERIRHQRQDYLHKIANYLVWKRYNN |
| | KGKRIETRDLGYQRGFPVTCKEMNKFMF |
| 254 | MLDPNQEQLSMMTVISGACRYVFNKALEIAVKNHLAGEKYVPYNKTAPLLVQWKSQENLSWLKLAPS |
| | QSLQQSLKDLDRAFHGYISRKSGFPKFRKKGTDESFRFPQQRVKVDEGNKKVYLPKIGWVRYRKSRDIIG |
| | EIKNITISQSANKWYVSFQTQIEVPDPVHTSNSTIKVTLSDEGTIFLSDGKKYALPATYSKHFNQLNKLIRQ |
| | KHRKIKNSQSWLAFHHSTILKKAKLRNILIDFLHKTSTLICNNHAKISVDTKKGNSARKTKPLPINFKPYEF |
| | LRQITYKQSWNGGSVCMEQS |
| 255 | MLETTRTYRAKIVNHSQVSDNLDDCGHSVSKLWNVARYHAQQEWDDTGEIPSEADLKRELKDHERYS |
| | DLHSQSSQRVLEELAESFNGWFKKRKNGDTDANPPGYRKRGDNHPRSTVTWKQNGIKHDSKHNQLRLS |
| | KGFNLKNHRSDFILCEYETRPDVTVENIQQVRAVWNGDHWELHLVCKVKIPVEDAPGDNTAGIDLGISN |
| | YLAIAYDDGEAELYPGNVLKQDKHYFTRDEYDTEGENGPSRRALRTRQKLSRRKDHFLHALAKHIVEQ |
| | CIDHEVGHIAIGDLSEIREDENGDSRNWGRSGNKKLHGWEFDRFTTLLEYKAEEHGILV |
| 256 | MITYKTMQIWVKKGHRMHPYFTEMCQNAKNMYNSTNFYIRQIFTGLTQEKELHPLQTEVLNTLQKHLP |
| | RMNDNQLLAYQKKIAKEKAKPVQKQKEVKCNLFEKPSKEKPYVDYHLLDALFKSISLNDYRSLPTQSSQ |
| | GIMKTVFQNWKSFYASLKEYKMNPTKFKARPRIPGYSRSKEKEVLFSNQDCVIKENKFLKFPKTKEIDNV |
| | ATIVTNTGRIPVLMKGKNIKSVNQRYNQLRAHFMGILRQGKNTNEGPFTSKRLEQINRKRFNQIKDLFHK |
| | ASHQIEKIALEEDVDTIIIGQNKEWKQQSNMGKRNNQSFTAIPHSLLIQMIKYKAARHGIKVIVNEESYTS |
| | KASFLDHDEIPVYGEVDLKKSFSGKRMKRGLYCSKNGTIINADVNGAANIMRKVFPKAFNETFACVQAL |
| | LQPISLLLK |
| 257 | MAEQIEEVPAELIQTRVYELHPNKTMRRVLDEACDYRRYCWNQGLALWNKMYKARQALKSSLASDSK |
| | KLTEEQKVLLKEKPSPSERRVRNMLVTDKKDWQYTQSARILQLAISDLGKAWNNFFDKAQPGWGKPKF |
| | RSKREARQGFKSDQSKIKDGILYLERAKESSVPKDQWRGFKLSEKPLSDEFGTVSYFKEKGRYYVAIPYK |
| | IKAEDVKLPDKTGKATAVDVNVGHFDYTGGRVNVLPKKLDRIYKKIKPLSKEACQKASRKWRSCLQNR |
| | ELLEDESQASSMLS |
| 258 | MRTIHFTLERCRLLYNRLLEERILAYKTEGKSLNYDQANTFNERKQHIPALKQVHSQVLQDVAKRLDKA |
| | FQAFFRRVKHGETPGFPRFKPQQQYDSFTYPQGGHAIKGNKVRLSKIGDVKIKLHRQPQGKIKTCTITVK |
| | NGKYYACFSFEVDPQQLPVSDEKVVLILACCILQLLQTAQRLRHQSNCEETKCRLKQLLTVCNTQETRFL |
| | IAERRLFTFWPNCMKRWRISIRIMHIRFPDNW |
| 259 | MRQFGHRARLALTSAQIRLMDDQAHAARTMWNCLHDWWTMLPKEKRSLAAADAATRQARKEIDWL |
| | GVLPAQAAQAVLNTYFQVWRNCWDGRADEPNFKARSRTVMSVDIPQGRDLNISRVHRRWGMVQIPRI |
| | GRIRFRWTKDLPVGKRANTENLITGARLVKDALGWHIAFRYDQIKQLRARATRRAVDWQHKTTTDIAR |
| | QYGTVVVEALTITNMVNSAKGTIEEPGKNVAQKSGLNRSISQEAWGRTVTMLTYKTARQGGTLVKVPA |
| | PGTSQRCSACGFTTPGSRQASPWRQARRRKVGRNLPRLRGRALQGGEPDAAEAVGEETGRRAQSSTGD |
| | VPVHHGGEVRSVRGGAGRLRRGRRAPGDGAGARRRARAAARGGLGGGSRGRRAA |
| 260 | MGIRRTYKVQIGRGHGLYPWCETITSLANNLYNACRFRQRQLITAARKTDRELTDNEKGVIAEFLSVLNC |
| | NGRSGLPACPRYETFDTVMKLTKNPDYYAKGLPRQSAQQVLKRSCADVDNFFAAVKAWKDRGCPEGE |
| | RPKFPGYKRKGGHGAVAITNQDCTLKEGRDGNLIAGLPFAKSMPLKIGFPLGRLKQAEVCPDNGVYVIA |
| | FSFELDLEVPVPVHPASWIAAIDFGVDNLMAVTNNCGLPCLLYKGGIVKSTNQGYNKRLAQIMQEEMK |
| | KPGCPKNKEGKPWFVPTEESMGMTLRRNNIVHDFMHKAAKHLVLWCVENRIDTIVGVNAGFKQEVNIG |
| | HINNQNFVQIPFAYLRSCIKYLCEEQGILYVKREEDLEYDQMSLVEYIDPFTGEPVRKNKK |
| 261 | MAKENPSNYKTLQIWIKKGHRMYSYFQECCHNAKNMYNTTNFYIRQVYTGLTQEKELQPLQKEVLANI |
| | HKNIGKMNDTQLLAYQKKLEKEKLKPKEEQKEITCNLFSEPNFEKPYVDYNFLDALFKAMIQNDYRALP |
| | TQCSQSIMKGVFQNWKSFFASLKDYKKNPNKYAGMPRIPKYIRSSEKEILYTNQDCIIKNSRFLKFPKTKL |
| | QLNIGKLGFTEGKLKQVRVIPKYNEYVVELVIDVPSEQQMIEENARYMSIDLGIDNLATIVTNTGMKPVL |
| | VKGKHVKSINQYYNKMKSHFTSILRNGKQTNEGPFTSKRIEKLHQKRYLKI |
| 262 | MTEQIEEVPAELIQTRVYELRPNETMRRVLDEACDYRRYCWNQGLALWNEMYKARQALKSSSASDSK |
| | KLTEEQKVLLKEKPSPSERRVRNMLVADKKDWQYTQSARILQLAISDLGKAWNNFFDKAQPGWGKPKF |
| | RSKREARQGFKSDRSKIKDGILYLERARGSRVPKDQWRGFKLSEKPLSDEFGVVSYFKEKGRYYVAIPY |
| | KIKAEDVKLPDKTGKATAVDVNVGHFDYTGGRVNVLPKKLDRIYKKIKHNQRQLAKKASPKWRSCLR |
| | KQELLEDESQASSVLSQGKQYPKRLDAQIYDRTG |
| 263 | MTKENPSNYKTLQIWIKKGHRMYSYFQECCHNAKNMYNTTNFYIRQVYTGLTQEKELQPLQKEVLANI |
| | HKNIGKMNDTQLLAYQKKLEKEKLKPKEEQKEITCNLFSEPNFEKPYVDYNFLDALFKAMIQNDYRALP |
| | TQCSQSIMKGLFQNWKSFFASLKDYKKNPNKYAGMPRIPKYIRSSEKEILYTNQDCIIKNSRFLKFPKTKL |
| | QLNIGKLGFTEGKLKQVRVIPKYNEYVVELVIDVPSEQQIIEENARYMSIDLGIDNLATIVTNTGMKPVLV |
| | KGKHVKSINQYYNKMKSHFTSILRNGKQTNEG |
| 264 | MEGIKEYRTYQIRIKKGHKLYEYFDKLCMNSNNLYNTTNFYIRQVYTAINNKKNLQPLQKEVMETIYQN |
| | LNKMNDKQTIAYFKKLRKEKTRPNEIRKEMILNLFEAPSKEKSFLGYNFLDCLFKTIKQKDYYSLPGQIN |
| | QQTIKNVVQNWKSFFSSLKDYKENPHKYKERPSIPGYLPKGSRKEVVLSNQICKIVGEKFLRFPKTKTQL |
| | NIGKLVNLKGTFQQIRIVPKYGDFIVELIYLVGDKREVVAKKEHCMSLDLGVDNIVTAVFNLKIVPILFKG |
| | GKIKAINQWYNKLRSLYYAAIRNGKGPKEGGFHSKRLVKLERDRHLKIKDLLHKVSFNIVKIAKAHQIDT |
| | IVIGKNKEWKQNSNLGKVNNQKFVQIPHTLLIELITYKANAKGIAVIVTEESYTSKASFLDGDHIPTFNPG |
| | NKEPHIFSGKRVHRGMYHSKHNILLNAHVNGAANILRKVVPKAFANGIAAVCSQPLVVNVQ |
| 265 | MLLTYKFRICPSKQQEQKLLFTLDKCRFTYNKLLEILNKQEKINQSEIQAKIPKLKQEYSDLNEIYSKTLQ |
| | YECYRLFSNLRALSRLKKNGKRIGKLRYKGKDWFKTFTYNQSGFVLGIKNKRYNKLHLSKIGILQIRTHR |
| | IINGSIKQVQIKKECSGKWFALLCVHKGEEKPKERTNKSIGIDLGTINFIYDSDGNHIDAPKFLSKSLKKLA |
| | GEQRKLSKKKKGSKNRIKQKINVARIHECIFNQRNDFLHKISRYYVNNYDF |
| 266 | MLKKYANKSLTQRESKQSSPEINSFKALINKKTAFFFMLLTYKFRIYPSKQQQEKLLFVLDKCRFTYNKL |
| | LEILNKQEKINQSEIQAEIPKLKEQYPDLNEIYSKTLQYESYRLFSNLRALSRLKKNGKKIGCLRFKGKDW |
| | FKTFTYNQSGFVLEIKNKKYNKLHLSKIGSIPIRTHRVINGSIKQVQIKKECSGKWFALLCVHMNEPKQRE |
| | KTKKSIGIDLGTINFIYDSDGNHADHPKFLNKSLKKLAAEQRKLSRKKKGSNNRTKQKINVAKIHEAICN |
| 267 | MIRTHIFACNIDRKLADSLNRESGRIYTQVMVEQWRTFRHAGHWISPRGVEKLADFYDKQAGQKPLLHA |
| | HSVDAAQQGFPKACKTAKACKNIGLNSSYPHKRKPYRSTIWKNTGISKTEDEKLQLALARGQSPIFIELPP |
| | NLKNLVKECYVEMRLVYNKNHKFYQWHVVVDDGMDSKVASGTNVIGIDLGEIHPVAATDGETSVVFS |
| | ARALRSVNQYSNKWLASFQSKISKKKKGSQSYKRMMARKRKFLAKQARRRKDIEQKVSRAVVDYAVE |
| | RNCNEIAIGDVRKVANKCKLGKKSNQKVSNWSHGKIRTMIEYKANAEGISVTMVKEHYTSQTCPNLDC |
| | QHKYKPSGRTYRCPVCGFVGLSHLR |
| 268 | MSYRWCWVWVVPRRRDPAAPRVVHRTARVAVRVTPGQRRRCFGLLRSAGDVWACLLEVNAWRRRR |
| | RDAPLVGYQQLCRELSGSGPGTLGELDTTGARSVLRRFSDAWFAAAKRRKDGDLSARFPRRRRGLVPV |
| | RWYHGTFTLDGRRARIPTAKGTAPLWIRLAREVPYPAEQVRSITLLCEGGRLFLDVTAEVPLAVYPPGEK |
| | PDPARFAGVDLGIIHPYAVAGPDGEGLLVSGRAIRAEHRMHLADTKARRRAVARRAPKRGEQGSRRWR |
| | KYRRRARLVEGRHRRRVRQAQHEAARQVVSWAVERRVGVLHVGDPRGVLDIAAGRRHNLRLRQWQI |
| | GRLLQVLTDKATLAGITSGWSTNAAPRPPAPPAAHAFRNLVAGPCPARAVDSPGTATWSRPPASPPAPR |
| | AADPPPRQPLLCCRRWSRTVEPAGTSPVPGGPGEDQLARGGPPHHLVGSRSPTRRGSTTTTEHPVNVRG |
| | HRTRGRMRRVSIGGRAGAGWGRIGSSRSAPRVGR |
| 269 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLARRKKYSKNWLKTKGKIDRVRSKAARIRLDNIHKATTAICKNHAVVEVVNLMGSVSAK |
| | NDNTPSTRYEFVRQLIYKQEWLGGEIIRRESKLL |
| 270 | MYVIALTVDLDQPRLKVGADLGKDRLQFLKSPAVEDTAAVFGYQHQMNMHSKHAVSSVPKVLAFVPR |
| | PDQNASMERQAFQFELMPNGEQPRQMSRSAGCVRYVYNQALALKKERYEKQEKLTRFELDKMLVGW |
| | KQETPWPSEAPAHALQQALLGLDRAYTNFFRKRAEFPKFHKKGIRDSFRESDPKCIKLDQVNRRIQVPKL |
| | GWVRYRNRREVLGEIPSVTVSLSAGKWFVSISTRREVEPPLHPSTSSVGLDWGMARFYTDAEHQDQL |
| 271 | MYVIALTVDLDQPRLKVGADLGKDRLQFLKSPAVEDTAAVFGYQDQMNMHSKHAVSSVPKVLAFVPR |
| | PDQNASMERQAFQFELMPNGEQPRQMSRSAGCVRYVYNQALALKKERYEKQEKLTRFELDKMLVGW |
| | KQETPWPSEAPAHALQQALLGLDRAYTNFFRKRAEFPKFHKKGIRDSFRESDPKCIKLDQVNRRIQVPKL |
| | GWVRYRNRREVLGEIPSVTVSLSAGKWFVSISTRREVEPPLHPSTSSVGLDWGMARFYTDAEHQDQL |
| 272 | MIRYKTEIKPNKKQIKEINKTINACRSVYNKFIEINKIRYDNGLKFLNHMKFSVWYNNEFIPNNEDKKWT |
| | KEVSTKTIKQAMANAENAYNRFWNYNSGYPNFKKKQSNGSYYLIGTIKIERHRIKLPNLKWVRLKEKG |
| | YIPKHNIKSATISKEFDRYYVSVLVDEEPKIIFKKLQTEGIGIDLGLKDTLFTPSGVKITDLRKNKRLIKLNK |
| | SLKRQQRKLSRKQKKSNNVKIVVQ |
| 273 | MKRLLRAYKTEIRPTEGQIILIHKTIGTCRYVYNLYLQKNREAYEATSSFLSGYDFSKWLNNEHATQDEF |
| | AWIKEVPSKAVKQAIMNADVAYKRFFKKLSSSPRSKRKSDYGSFYLVGTIHVKRHLIQLPKLGKVKLKE |
| | KGYIPFDGVKSATVSREGDRYFVSVLVEEPSRVVQKHGQTDGIGIDMGIKELLFDSDGNAVANINRSNQII |
| | KLTRSLKRQQRKLSRRVKGSENFKKQKVIV |
| 274 | MAEQVKEAPAELIQTRVYELCPNKTMRKVLDEACDYRRYCWNQGLDLWNEMYKERQALKSSLASDS |
| | KKLTEEQKVLLKEKPSPSERRVRNMLVADKKDWQYTQSARILQLAISDLGKAWNNFFDKAQPGWCKP |
| | KFRSKREARQGFKSDRSKIKDGILYLERARGSRVPKDQWRGFKLSEKPLSDEFGTVSYFKEKGRYYVAIS |
| | YKIKAEDVKLPDKTGKATAVDINVGHFDYTGGRVNVLPKKLDRIYKKIKHYQRQLAKKRVQNGAAAC |
| | ESKNYLKTKAKLQA |
| 275 | MMTVISGACRYVENKALEIAVKNHLAGEKYVPYNKTAPLLVQWKSQENLSWLKLAPSQSLQQSLKDLD |
| | RTFHGYISRKSGFPKFRKKGTDESFRFPQQRVKVDEGNKKVYLPKIGWVRYRKSRDIIGEIKNITISQSAN |
| | KWYVSFQTQIEVPDPVHTSNSTIKVTLSDEGTIFLSDGKKYALPATYSKHFNQLNKLIRQKHRKIKNSQS |
| | WLAFHHSTILKKAKLRNILIDFLHKTSTLICNNHAKISVDTKKGNSARKTKPLPINFKPYEFLRQITYKQS |
| | WNGGSVCMEQS |
| 276 | MTKGKDSTHSRKETKQLQALSRKRDAFLRDFFYKTAWYLVRYAKEQRVDVIVIGHNEEQKQNIRIGKQ |
| | NNQNFVSIPFCLFIKILRNTAAKVGIPVVDREESYTSKASLLDLDAIPTYRKGNAQTYTFSGKRVHRGLYK |
| | TNSGCVINADINGDGNILRKEYPYAFDGQDMSYLYKTTKVVSYTDIYAGAKSVCKEKYNQKSHEPGLGS |
| | RVNHRYRQDTRLIYRKLWGRSTFVWTGKKKTA |
| 277 | MSMQLTETVKLYPNKYQTELIKATMTEYISTVNHLVFDAINGRTITKITTADVNAILPSALCNQCIRDSKSI |
| | IRKYNKALRNSDTQVKLPVLKKMCCYINNQNFRISDDCISFPVIINGKSKRISVKTKISKRQKSIFSSSKLGT |
| | MRIVVKGHDLVAQIQNIRSTTRTSRKNNHNLHTWSFYRLATFIEYKAKLAGIEVEYVDPAYTSQICPICG |
| | RIQHAKDRNYTCRCGYQTHRDLLGAINICNSTEYIGNRYTA |
| 278 | MRTFKMIIDPTKNKKDAAFCQYFKTNTADSKCMYNTANFYIRNTMTGLKKSPEERTHLETEVLHYVFTG |
| | IQKANEVIGQKNMKKKFAELNLSKVGGMNSAVIAFSIVSQEPFQYPTEEKWFLSYGTLDAIFKFTDNPVY |
| | RRMNSQVNQNAIRKAVTAWQGYFESLKAYKKNPAGFTGKPKIPGYKQDEEYIAWFSKQVAKLKEEDG |
| | RCYIQFVNNPDRFEIGKASLYSDLKYVKTEVKAMYGKYYILITFDDKIAEVEAPENPKRILGLDPGVNNF |
| | LGVANNFGGVPFVMNGRAVKSANQRFNKKRAKLISSVTKGSDSKSSVKYSKHLNILSQKRESFLRDYFY |
| | KCAWYICRYAKAAGVDVIVMGHNDGQKQEIDLKDNVNQNFVSIPYTKFITILKAVASKCGIAVVIREES |
| | YTL |
| 279 | MKSIRTKLKLNNKQKTLMAQHAGYSRWCYNWGLSLWNAAYTDGYKPNPRKLREVFTNHTKPLYPWM |
| | KNLSSKVYQYAFINLGEAFKRFFQGLGKRPRFKKKGKSDSFTIDNCGKPIELNGWSHKLPFIGMVKTYEP |
| | IEATTQKITISRQAGDWYLSLSYEFTPSPTPTTTEVVGVDLGVKTLATLSDGKVFESVKSYRRFETKLSRL |
| | QYLNRNKIIGSAN |
| 280 | MLRAYKTEINPSFEQCQTINQTIGTCRWIYNKFIETNQYLYEKEKSYMDGYTFSKWMNNVYLPSHPDKH |
| | WVKQSASKAIKQSIMNAHRAYQTFFKNKQGYPKFKKKSGIGSYYLIGTIHVQRHRIQLPKLGWIKLKER |
| | GYIPTNNIKSATIVKEYDRYYVSVLVDQPPPPIFKPEQTEGIGIDLGLKEAVFTPSGVKIRSFKTNQTIIKLD |
| | KSLKRQQRKLSRKKKGSRN |
| 281 | MLRAYRTEIDPSFEQRQTINQTIGTCRWIYNKFIETNQNFHKTGQSYMNGFAFSKWMNNVYLPNNPNKH |
| | WIKQSASKAVKQSIMNAHRAYQTFFKNKKGYPKFKKKSGIGSYYLIGTIHVQRHRIRLPKLGWIQLKEK |
| | GYIPTNNIKSATVIREFDRYYVSVLIDCEPSPLFKPKQTEGIGIDLGLKEAVFTPSGVKIRSFKTNQTIVRLD |
| | KSLKRQQRKLSRKKKGSHNWYKQLLKVQRLYRRIKNKKEISNAKAYSLLFAQIRNLLRLKI |
| 282 | MTEQIEEVPAELIQTRVYELRPNETMRRVLDEACDYRRYCWNQGLALWNEMYKARQALKSSLASDSK |
| | KLTEEQKVLLKEKPSPSERRVRNMLVADKKDWQYTQSARILQLAISDLGKAWNNFFDKAQPGWGKPKF |
| | RSKREARQGFKSDRSKIKDGILYLERARGSRVPKDQWRGFKLSEKPLSDEFGVVSYFKEKGRYYVAIPY |
| | KIKAEDVKLPDKTGKATAVDVNVGHFDYTGGRVNVLPKKLDRIYKKIKHNQRQLAKKASPKWRSCLR |
| | KQELLEDESQASSVLSQGKQYPKRLDAQIYDRTG |
| 283 | MIKKQAFKFLLEPNKSQLSDFLAFAGSCRFVYNKGLALLNENYRSGKKFIGYNQLASELVEWKHEESLS |
| | WLKEAPSQCLQQSLRDLDRAFRNFFTGKAQYPKFKKKGRHDSFRIPCQRVRVDQEKKLVSLPKVGWVK |
| | YRKSREIIGDLKNVTISMKHGKWYISFNTEHTVPDPIHPSDIKTTIVLNNENSVHLSTRVGGANTYQAEEK |
| | KKLVRLNKILARRKKHSNNWLKTKGKIDSVILKSARIRLDNIHKATTAICKNHAVVEVVNLMDSVSDKN |
| | DNTLSMRYEFVRQLIYKQEWLGGEIIRR |
| 284 | MLRAYRTEIDPSFEQRQTINQTIGTCRWIYNKFIETNQNFHKTGQSYMNGFAFSKWMNNVYLPNNPNKH |
| | WIKQSASKAVKQSIMNAHRAYQTFFKNKKGYPKFKKKSGIGSYYLIGTIHVQRHRIRLPKLGWIQLKEK |
| | GYIPTNNIKSATVIREFDRYYVSVLIDCEPSPLFKPKQTEGIGIDLGLKEAVFTPSGVKIRSFKTNQTIVRLD |
| | KSLKRQQRKLSRKKKGSHNWYKQLLKVQRLYR |
| 285 | MIKHQAFKYMLDPNQEQLSMMTVISGACRYVENKALEIAVKNHLAGEKYVPYNKTAPLLVQWKSQEN |
| | LSWLKLAPSQSLQQSLKDLDRAFHGYISRKSGFPKFRKKGTDESFRFPQQRVKVDEGNKKVYLPKIGWV |
| | RYRKSRDIIGEIKNITISQSANKWYVSFQTQIEVPDPVHTSNSTIKVTLSDEGTIFLSDGKKYALPATYSKH |
| | FNQLNKLIRQKHRKIKNSQSWLAFHHSTILKKAKLRNILIDFLHKTSTLICNNHAKISVDTKKGNSARKTK |
| | PLPINFKPYEFLRQITYKQSWNGGSVCMEQS |
| 286 | MTEQIEEVPAELIQTRVYELRPNETMRRVLDEACDYRRYCWNQGLALWNEMYKARQALKSSLASDSK |
| | KLTEEQKVLIKEKPSPSERRVRNMLVADKKDWQYTQSARILQLAISDLGKAWNNFFDKAQPGWGKPKF |
| | RSKREARQGFKSDRSKIKDGILYLERTRGSRVPKDQWHGFKLSEKPLSDEFGVVSYFKEKGRYYVAISY |
| | KIKAEDVKQPDKTGKATAVDINVGHFDYTGGRVNVLSKKLDRIYKKIKHYQRQLAKKRVQNGAAACE |
| | SKNYLKTKAKLQA |
| 287 | MIVLEYKVKGKPNQYQAIDQAIRTTQFVRNKAIRYWMDNSRELKIDRFALNKYSTTLRNEFPFVADLNS |
| | MAVQSASERGWSAISRFYDNCQKKISGKKGYPKFQKDCRSVEYKTSGWALHPTKRQITFTDKNGIGKLK |
| | LLGKWDIQSYNVKDIIRQWIEYFAAKFDKLAIPVAPHYTSQKCSNCGVIVKKSLSTRTHVCNCGCELHRD |
| | TNAAINILNLGKQARGGHPRSNANGLETSTLLGETLVAARI |
| 288 | MAKENPSNYKTLQIWIKKGHRMYSYFQECCHNAKNMYNTTNFYIRQVYTGLTQEKELQPLQKEVLANI |
| | HKNIGKMNDTQLLAYQKKLEKEKLKPKEEQKEITCNLFSEPNFEKPYVDYNFLDALFKAMIQNDYRALP |
| | TQCSQSIMKGLFQNWKSFFASLKDYKKNPNKYAGPPRIPKYIRSSEKEILYTNQDCIIKNDRFLKFPKTKL |
| | QLNIGKLGFTEGKLKQVRVIPKYNEYVVELVIDVPYEQQMIEENARYMSIDLGIDNLATIVTNTGMKPVL |
| | VKGKHVKSINQYYNKMKSHFTSILRNGKQT |
| 289 | MDQIKQLRIYPPEKGSCKIIVVYEVPDQEELPQNGHELSIDLGLHNLMTCYDSENGNTFILGRKYLGLERY |
| | FHKEIARVQAQWYGQQSGKGVKHPTTSKHIRKLYKRKHDSVTDYLHKVTRYLAEYCREQGITCVIAGD |
| | XENKIFRGMDQIKQLRIYPPEKGNCKIIVVYEVPDQEELPQNGHELAIDLGLHNLMTCYDPGNGKTFILG |
| | RKYLALERYFHKEIARVQAQWYGQQSGKGVKHPVTSKHIRKLYKRKHDSVTDYLHKVTRYLAEYCRE |
| | QGITCVVAGDIRNIRREKDLGRRTNQKLHSLPYNRIYIMLEYKLKRYGIRFIKQPANKKSRLN |
| 290 | MAEQVKEAPAELIQTRVYELCPNKTMRKVLDEACDYRRYCWNQGLDLWNEMYKERQALKSSLASDS |
| | KRLTEEQKVLLKEKPSPSERRVRNMLVTDKKDWQYTQSARILQLAISDLGKAWNNFFDKAQPGWGKPK |
| | FRSKREARQGFKSDQSKIKDGILYLERAKESSVPKDQWRGFKLSEKPLSDEFGTVSYFKEKGRYYVAIPY |
| | KIKAEDVKLPDKTGKATAVDVNVGHFDYTGGRVNVLPKKLDRIYKKIKPLSKEACQKASRKWRSCLQN |
| | RELLEDESQASSMLS |
| 291 | MIKKQAFKFLLEPNKGQLSDFLAFAGSCRFVYNKGLALLNENYRSGKKFIGYNQLASELVEWKNEASLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLTRRKKYSKNWLKTKGKIDRVRAKAARIRLDNIHKATTAICKNHAVVEVVNLMDSVSAK |
| | NDNTLSMRYEFVRQLIYKQEWLGGEIIRRESKLL |
| 292 | MIKKQAFKFLLEPNKGQLSDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLTRRKKYSKNWLKTKGKIDRVRAKAARIRLDNIHKATTAICKNHAVVEVVNLMDSVSAK |
| | NDNTLSMRYEFVRQLIYKQEWLGGEIIRRESKLL |
| 293 | MIKKQAFKFLLEPNKGQLFDFLAFAGSCRFVYNKGLALLNENYRSGKKFIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKTTIVLNNVNSVHLSSGVGGDNTYQAEEK |
| | KKLIRLNKTLTRRKKYSKNWLKTKGKIDRVRSKAARIRLDNIHKATTAICKNHAVVEVVNLMDSVSAK |
| | NDNTLSMRYEFVRQLIYKQEWLGGEIIRRESKLL |
| 294 | MLELNAWRRQRRDTPLASYQELCRELAASGPGVFGELDSTGARSVLRRFSDAWFAAAKRRRGGDAAA |
| | RFPRRRRGLVPVRWYHGTFTLDPGGRRVRIPAARGGQPLWLRLARSLPYPVDQVRSVTLLAEGGRLFLD |
| | VTAEVPVTVYEPGCGPDPARTAGVDLGIIHPYAVAGPDGQALLVSGRAIRGEHRMHLADTKQRRRAVA |
| | GRAPTRGQRGSRRWRKYRRRARAVDGRHARRVRQAQHEAAKTVVSWAVGQRVGTLHMGDPRGVLQ |
| | VAAGRRHNLRLRQWQIGQLIRILADKATVAGITFTSSTNAAPRPPARAAGGGSRNHPGGS |
| 295 | MISYRTEIKPNKKQIREINKTIDACRTVYNKFLEVNKIIYENNKSFMSHTKFSVWYNNEFIPNNEDKKWT |
| | KEVNTKATKQAMANAENAYKRFWKNNNGFPKFKKKQNNGSYYLIERIHVERHRIKLPNLKWVKLKEK |
| | GYIPSSNIKSTTIIKDGNRYFVSVLVDEEHKTIFKPLQTEGIGLDLGLKDTLFTPKGVHITDLRKNKKLINLD |
| | KSLKRQQRKLTRKQRKSNNWFKQLLKVQRLYRKISNIKKDIKRKKVLEII |
| 296 | MIKKQAFKFLLEPNKGQLSDFLAFAGSCRFVYNKGLALLNENYRSGKKIIGYNQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKSTIVLNNVGSVHLSSGVGGDNTYQAEEK |
| | KKLIRLNKILTRRKKHSKNWLKTKGKIDRVKSKAARIRLDNIHKATTAICKNHALVEVVNLMDSVSDKN |
| | DNTLSMRYEFVRQLIYKQEWLGGEVIRRESKPL |
| 297 | MIKKQAFKFLLEPNKGQLSDFLAFAGSCRFVYNKGLALLNENYRSGKKFIGYIQLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPDPIHPSDIKSTIVLNNVDSVHLSSGGGGDNTYQAEEK |
| | KKLIRLNKTLTRRKKHSQNWLKTKGKIDRVKSKAARIRLDNIHKATTAICKNHAVVEVVNLMDSVSAK |
| | NDKTLSMRYEFVRQLIYKQEWLGGEIIRRESKLL |
| 298 | MLTITHRYRIYPDATQKQQFIDWMEVCRGAFNYALREIKDWCNSRKCLIDRCSLEKEYILPAELKFPSEIQ |
| | QLNNLPRAKKEFPRLSEVPSQVLQQAIKQLHKAWEYFQKRGFGFPRFKKYGQFKSLLFPQFKENPVPNL |
| | HVLLPKIGTIPINLHRPIPTGFVVKQVRILRKADRWYAWKRGNFFGEVDARGTSQECPECGGEVRKDLSV |
| | RIHNCPHCGYKTDRDVAAGQNIRNRGIKLISTVGQTGKETACADVLPGAEETQSRQVSKSPERSAALSLP |
| | KGRRGVTRKPKK |
| 299 | MLNLTYNYRIYPGLDQEAQMLDWLEQCCRVYNYAFAERKDWIGSRKCPVNACSIKQEYIISADAPYPD |
| | YYKQQNALTRAKREIPELKAVHSQVLQDALKRLDKSFKFMQKRGFGFPRFKKFGQYRSFVFPQFKFKM |
| | TSRGILAKHCLDAAWGSFLEILKWVAWKRGVYFARVDPNGTRQTCPQCGVHTGKKELGERVHHYSEC |
| | GYTTDRDVAAAQVVMQRGLVLAADGQSVMLPAEEGCLGTPMKQENPTARKGSPRSTR |
| 300 | MQTLEYKIKASINQYKAIEEAIRTTQFVRNKSIRYWMDSPQDAKINRFSLNKYSTELRNKYQFVSDLNSM |
| | AVQSASERAWISIQRFYDNCKKSSKGKKGYPKFQKDNRSVEYKTSGWKLHPSKRKITFTDKKGIGDLKL |
| | LGKWDIHLYPLKSIKRVRIVRRADLAKSISDVGWYLFRQWIEYFASKFGRIAIAVAPQYTSQKCSNCGRI |
| | VKKSLSTRTHVCVCGCELHRDTNAAINILNLAKQARAGQARSNATGDATSTLVAERLSQQVASMNVES |
| | PRL |
| 301 | MKSIRTKLKLNNKQKTLMAQHAGYSRWCYNWGLSLWNAAYRDGYKPNYRKLREVFTNHTKPLYPW |
| | MKNLSSKVYQYAFINLGEAFKRFFQGLGKYPRFKKKGRSDSFTIDNCGKPIELNGWSHKLPFIGIVKTSEP |
| | IEATTQKITISRQAGDWYLSCSYEFHSHTTPKKTDVVGVDLGMKTLATLSDGKVFESVRAYQKFEAKLS |
| | RLQYLNRHKQVGSANWRKAQLKIARLLRKVANIRQDALHKLTTYLAKVRLVPVRSL |
| 302 | MITLTYQYKLKPNKQQEADINLMLDVCKSVYNYGLRERKDWLNSRKSPINSCSIVSEYIIPADTPYPNYN |
| | HQAKNLTIAKKTNTKLKSVNAQVLQQTLKTLERAFSDMKYLGKGFPRFKKKLRSFVFPAMLKNCLGNN |
| | RVKLPQLGWIKIRQSRQYPDGFQAKQARIVKKATGYYLMIIFTSSESAPDNPVGKKSLGIDAGIESFVATS |
| | TGKLIKSPKFLLSQLR |
| 303 | MKSIRTKLKLNNKQKTLMAQHAGYSRWCYNWGLSLWNQAYTDGYKPNTRKLREVFTNHTKPLYPWM |
| | KNLSSKVYQYAFINLGEAFKRFFQGLGKRPRFKKKGKFDSFTIDNCGKPIELNGWNHKLPFIGIVKTYEPI |
| | EATTQKITISRQAGDWYLSLSHEFSATPTPKTTDVVGVDLGVKTLATLSDGKVFESVRAYQKFEAKLSRE |
| | QYLNRHKQVGSANWRKAQLKIARLHRKVANIRQDALHKLTAYLAKVRLVPVRSL |
| 304 | MLTGRRYLLALTDVQTGQAERFGAICRAVWNTGLQQRREYRRRGAWINYVQQARQMAEAKKDLDCS |
| | WLAEAPSHIPQQTLRDLEKACQAHGTGKVRWRSKSRTAPAFRFPDPNQIRVERLNRRWGRVRLPKLGW |
| | VRFRWSRPLGGPIRNATVARDGGRWYISFCVEDGVTGVAPTDAPGVNVRQKAGLNRAILNKGWGGVL |
| | LALEHTARYHGATVVSVNPAYTSQRCSRCTLVDANSRKSQAEFTCTGCGHRDNADVNAAKNMP |
| 305 | MNYNYRYRLMPTDSQRETLDYHRDTCRQLYNHALYRFNQIPEDEGTVKQRVRTIRDELPDLKDWWDA |
| | LTDVYSKVLQPTVMRIAKNINALGRLKEQGYKVGELRWKSPREFRSFTYNQSGFELDKNGGQTVLSLSK |
| | LADIPIELHRPLPEDATVKEVTLKKEKTGEWFAIFGIEMDTEPPAKPPLEDIDAENMVGIDVGILKYAHDT |
| | DGTAVESLDLSEERDRLEREQRKLSRKAYESNNWERQRRKVAECHLDIKRKRRDFLHKLSAYYAREYE |
| | LVRSKTST |
| 306 | MKTLKLRIKDKHCKVLDQLASEVNFVWNYVNDLGFRHLKRKGEFLSAFDIAKYTKGTSKECNLHSQTI |
| | QAVTEELVTRRKQFKKAKLKWRVSNKKSARRSLGWVPFKKVAIKYANGYVQYGKHQFKLWDSYGLS |
| | KYTFSDGTVISNPKFYRKYEQTLGIAQRARNKKRVRALHAKIANSRKDHLHKASTKLVNENALIIVGDL |
| | NAKKLVKTKMAKSVLDTGFSALKTMLKYKCENAGVLFEEVQEAYTTQICSCCGEITSSSPKGSTDLGIRE |
| | WECMSCGTVHDRDINSALNILALGHKRLAVGITLF |
| 307 | MKYLTGYDFHKWINKEYLPNNPDKLWIKEVYSKSTTRAMQNADKAYKNFLQGNSRFPKYKKKQTNGS |
| | YYLWGNMEVERHRIKLPKLKWVKLKRKGYIPTDLKVVSATLTKEVDRYYISVMFERELNVIFKKPQTEG |
| | IGIDLGLKDTLFTPSGVHITDLRKNQKLIKLTKSLKRQQRKLSRKQSKSNNWFKQLLKIQRLYRKISNIKK |
| | DIKQKKILEVESYQIVSNK |
| 308 | MLVGRKYRLEFDFGQRAFAERLGGICRAVWNTGLEQRREYRRRGQWINYAEQCKQLAEAKKDPYCG |
| | WLADAPAQVIQQTLKDLDQACRKHGTWKVRWKSKAKWRPSFRFPTAQHLPVERIGRRWGRVSLPKFA |
| | VKPKPDPGRPGRFLCNGAAAKSGLNRAILDKGWYGLEVALRSKARYTGSVIHKINPVYTSQTCPESACG |
| | KVDEKSRKSQAIFSCTSCGHTEHADIVGARNIKSKGQAAGLVVSGRGDPPGSAKRQAPRSTARAAQAAR |
| | AAA |
| 309 | MAEQIEEVPAELIQTRVYELHPNKTMRRVLDEACDYRRYCWNQGLALWNEMYKARQTLKSSLSTDSK |
| | KLTEEQKVLLKDKPSPSERRVRNMLVADKKDWQYAQSARILQLAISYLGKAWNNFFDKAQPGWGKPK |
| | FRSKREARQGFKSDQSKIKDGILYLERAKESSVPKDQWRGFKLSEKPLSDEFGVVSYFKEKGRYYVAIPY |
| | KIKAKDIKLPDKTGKATAVDVNVGHFDYTGGRINVLPKKLDKIYGKIKHYQRQLVKKQVKNGEAACES |
| | ENYLKTKAKLQACYRKASN |
| 310 | MAEQVKEAPAELIQTRVYELRPNKTMRKVLDEACDYRRYCWNQGLALWNEMYKARQTLKSSLSTDFK |
| | KLTEEQKVLLKDKPSPSERRVRNMLVADKKDWQYTQSARILQLAISDLGKAWNNFFDKAQPGWCKPK |
| | FRSKREARQGFKSDRSKIKDGILYLERARGSRVPKDQWRGFKLSEKPLSDEFGVVSYFKEKGRYYVAISY |
| | KIKAEDVKLPDKTGKATAVDVNVGHFDYTGGRVNVLPKKLDRIYKKIKHYQRQLAKK |
| 311 | MEKAYSYRFYPTPEQESLLRRTLGCVRLVYNQALHERTQAWYERQERVGYSQTSSMLTNWKKQEDLD |
| | FLNQVSCVPLQQGLRHLQTAFTNFFVGRAKYPNFKKKHQGGSAEFTKSAFKFKNGQIYLAKCLEPLAYK |
| | CRWYGRNYIEIDRWFPSSKRCSNCGHIVEKMPLNIREWDCPNCGTHHDRDLNASKNILAAGLAVSVCGA |
| | SVRPEQSKSVKATAKKQKPKL |
| 312 | MIKHQAFKYMLHPNQEQLSMMTVISGACRYVFNKALEIAVQNHIAGEKYVPYNKTAPLLVQWKSQESL |
| | SWLKLAPSQSLQQSLKDLDRAFHGYISRKSGFPKFRKKGTDESFRFPQQRVKVDEVNKKVYLPKIGWVR |
| | YRKSRDVIGEIKNITISQTANKWYVSFQTQIEIPDPVHTSSLTAKVTLSDEGTILLSDGKKYALPETYSRHF |
| | NQLNKLIRQKNRKIKNSQSWLAMHHSIILKKAKLRNILMDFLHKTSTLICNNHAKISVDTEKGNSARKTS |
| | PLPVNFKPYEFLRQLKYKQSWNGGSVCVEQT |
| 313 | MKQQVSFKFRLKPDGQQERQMRRFAGACRFVFNRALALQNENHEAGKKYIPYTKMASWLVEWKKDT |
| | ETEWLKDSPSQPLQQSLKDLERAYKNFFQNRAAFPRFKKRGQNDAFRYPQGVKLDQENSRIFLPKLGW |
| | MRYRNSRQVTGIVKNVTVSQSCGKWYISIQTEREVSTPVHPSASMVGLDAGVAKLATLSDGTVFEPVNS |
| | FQKNQKKLARLQRQLSRKVKFSNNWQKQKRKIQ |
| 314 | MRVAFVYRLYPTREQMRTIHFTLERCRLLYNRLLEERILAYKTEGKSLNYDQANTFNERKQHIPALKQV |
| | HSQVLQDVAKRLDKAFQAFFRRVKHGETPGFPRFKPQQQYDSFTYPQGGHAIKGNKVRLSKIGDVKIKL |
| | HRQPQGKIKTCTITVKNGKYYACFSFEVDPQQLPVSDEKVVLILACCILQLLQTAQRLRHQSNCEETKCR |
| | LKQLLTVCNTQETRFLIAERRLFTFWPNCMKRWRISIRIMHIRFPDNW |
| 315 | MKRLQAFKYELQPNGEQARSMRRFAGSCRFVFNKALAMQKAIYEGGEKKLGYAGLCKELTTWKTQPE |
| | TAWLKEIHSQVLQQSLKDLERAYKNFFDKRADFPRFKKKGMGDSFRYPQGCKLDQSNSRVFLPKLGWL |
| | KYRNSRDVLGTVSNITVSANGGKWFVSIQTEREVEQPVHPATSIVGIDVGITRFATLSDGSHIEPLNTFRK |
| | HQQRLARYQRAMSRKTKFSSNWKKAKARVQKIHTRIANVRKDFLHKTTTTISKKPRDCVHRGFAGTEY |
| | VQVRSRQQRFARAQCQSQIWPE |
| 316 | MKRLQAFKYELQPNGEQARSMRRFAGSCRFVFNKALAMQKAIYEGGEKKLGYAGLCKELTTWKTQPE |
| | TAWLKETHSQVLQQSLKDLERAYKNFFDKRADFPRFKKKGMGDSFRYPQGCKLDQSNSRVFLPKLGW |
| | LKYRNSRDVLGTVSNITVSANGGKWFVSIQTEREVEQPVHPATSIVGIDVGITRFATLSDGSHIEPLNTFR |
| | KHQQRLARYQRAMSRKTKFSSNWKKAKARVQKIHTRIANVRKDFLHKTTTTISKKPRDCVHRGFAGTE |
| | YVQVRSRQQRFARAQCQSQIWPE |
| 317 | MKRRQAFRFNVRPTDTQERIFRQFAGAFRFVHNRALVLEIDRHASGKAHLGYVGTANLLPLWKRDPET |
| | VWLSGIHSQILRQSLKDLDRAYKNFFEKRAGFPKFRRKGEKDSFRFPQGARLDEPNARIWLPKIGWVRY |
| | RKSRTVLGTIKNVTVRRSGDRWFVSIQTEREIESPVHPNPGIVGIDLGVARFATLSDGTAIAPGRFFSRHEA |
| | RLKRLQRALSRKKKGSKTGRRSERSWPGSTGTWPTRGTTSCTRSRRRSAKATRSSWSKT |
| 318 | MKRRQAFRFALRPTDTQERIFRQFAGACRFVHNRALALEIDRHASGEARLGYVGTANLLPLWKRDPETV |
| | WLSGIHSQILQQSLKDLDRTYRNFFEKRAGFPKFRRKGENDSFRFPQGARLDEPNARIWLPKIGWVRYRK |
| | SRTVLGTIKNVTVRRSGDRWFVSIQTEREIESPVHPNPGIVGIDLGVARFATLSDGTAIAPGRFFSRHEARL |
| | KRLQRALSRKKKGSKTGRRSERSWPGSTGTWPTRGTTSCTRSRRRSAKATRSSWSKT |
| 319 | MKRRQAFRFTVRPTDTQERIFRQFAGAFRFVHNRALVLEIDRHASGKAHLGYVGTANLLPLWKRDPETV |
| | WLSGIHSQILQQSLKDLDRAYKNFFGKQAGFPKFRRKGWNDSFRFPQGARLDEPNARIWLPKIGWARYR |
| | KSRTVLGTIKNVTVRRSGDRWFVSIQTEREIESPVHPNPGIVGIDLGVARFATLSDGTVIAPGRFFSRHEAR |
| | LKRLQRALSRKKKGSKTGRRSERSWPGSTGTWPTRGTTSCTRSRRRSAKATRSSWSKT |
| 320 | MARKKAVKVLRKQKKRETMQRFTQKQNIGRACLTAKEFRLLQRMSHSSKALRNVGLYTMKQIYLNNN |
| | RMATVKEVDTAMQADINYPGVQSNSVQAIRRALFTEVKSFFKALEQWKKKPEKFTGRPKFPNYSRSTD |
| | KRIIEIYQVPKVDDNGYWIIPMNVAFRKKFGSIKIRMPKNLRNKKISYIEIVPKQKGRFFEVHYTYEMHVS |
| | QMKKPSTTTSNALSCDLGVDRLVSCVTNTGDTFLIDGKKLKSINQYFNKTIRNLQQKNMENGLSKRVVT |
| | NQMAELWHKREQQINGYISQTVGLLFKKVKVFNIDTVVVGYNAGWKQESDMGKKNNQKFVQIPFHKLI |
| | AAIENKCVKEGIRFLKQEESYTSKPVFLIKIRFPFGLRMIGRIIALVANESLMVCTKVKQEHVFMLILMVR |
| 321 | MKYQTQKILLTGNIDDETHAYLLWCCEQSNKLYNSVLFTIRQDYFEKCNYKTWFDKNDNYRRSPRLRR |
| | VKISYAQLCKDFKDDVHYQAIGGQQGQQTIKSVVEAIKGYNKLLPMWFSGELKDTPRIPSYRKRGLYQV |
| | AFTSQNIRYEPLEGICYLPIPNSQRKELETPSIIIPSGVNFQSEDIAEVRVIPSNGKLWAEYVYKTQLLKASN |
| | LDYSQGLGIDHGVDNWLSCISTKGKSFIVNGRKIKSINQRYNRLVAKQKQGKSQEYWDEKLDQATHKC |
| | NCQMRDAVNKAARFIINYCLKYQIGNIVFG |
| 322 | MVKTMAKKKAVKVLRKQKKRETLRRFTQKQNIGRACLTAQEFRLLQRMSHSSKALRNVGLYTMKQSY |
| | LNHNKMATVKEVDAAMQADMNYWGIQSNSVQAIRRALFTEVKSFFKALEQWKKNPEKFTGRPKFPNY |
| | SHSTDKRIIEIYQVPKVDENGFWMIPMSVAFRKKFGSIKIRMPKNLRNKNIYYIEIVPKQKGRFFEVHYTY |
| | EMHVSQMKKQPMTTSNALGCDLGVDRLVSCVTNTGDAFLIDGKKTKNPLTSTSTKRYVIYNKKMWKM |
| | DFQNEL |
| 323 | MDKKTYKLLRTLTHLSKDLYNLTLYTVKQHYELNGTFLPFVKAYHMVKDSEPYKLLPSQVAQQTMKIV |
| | ERNFRSFFHVLKERKKGNYNRPVRPPKYLPKNGHFILIFPYQSFRVKEDRIILTLGKNFAEKYGVKHLEIP |
| | LPKNVKGHRIKEIRILPRYNALWFEVEYVYEVLPEERDLDRSKYLAIDLGLDNFATCVSTTGTAFIIEGRG |
| | LKSFNRWWNKEKAKLQSQYDKQGVKFGKRMVWLLKKRKNVVNDFMNKAVSYIVNYCLENGIGNVVI |
| | GELKGVKQNTDLGRRNNQNFHYIPYGLFKQKLKAKCERYGINYIEVDEAYTSKVDALTLEPIEKREKYL |
| | GKRGETWTVPVFRWCFNKC |
| 324 | MARKKAVKVLRKQKKRETMQRFTQKQNIGRACLTAKEFRLLQRMAHSSKALRNVGLYTIKQSYLNDN |
| | KMATVKEVDTAMQADMNYWGIQSNSVQAIRRTLFTEVKSFFKALEQWKKNPEKFTGRPKFPNYSRSTD |
| | KRIIEIYQVPKVDENGYWIIPMNVAFRKKFGSIKIRMPKNLRNKKISYIEIVPKQKGRFFEVHYTYEMHVS |
| | QMKKQPTTTSNALSCDLGVDRLVSCVTTTGDAFLIDGKKLKSINQYFNKVIRNLQQKNMENGLSKRIVT |
| | N |
| 325 | MARKKAVKVLRKQKKRENMQRFTQKQNIGRACLTAKEFRLLQRMSHSSKALRNVGLYTMKQSYLNN |
| | NRMATVKEVDTAMQNDMNYSGIQSNSVQAIRRSLFTEVKSFFKALEQWKKNPEKFTGRPKFPNYSRST |
| | DKRIIEIYQVPKVDEKGYWMIPMNVAFRKKFGSIKIRMPKNLRNKNISYIEIVPKQKGRFFEVHYTYEMH |
| | VSQMKKQPTTTSNALSCDLGVDRLVSCVTNTGDTFLIDGKKLKSINQYFNKMICNLQQKNMDNGISKRI |
| | VTNT |
| 326 | MRLVERHVIKKNHRFYAEIDRLCFLSKNLYNYANYLVRQSFIFENTYRNYHDVQKTLQSQQDYQAMPA |
| | KVSQQVLMILDRNWISFKESNLAYKESPSKFKARPRLPGYKHKIKGRNVVVYTAQAIRKKQLKRGIINPN |
| | KTAIYLKTKVDTSKIKQVRLVPRLNHYVIEVIYEADKQQYELEENRYASIDIGLNNLATLTFNQAGIKPLL |
| | INGKPLKSINQYYNKVKSDLQSLLGENKSSQKLKKLCNKREFKINDYLHKASRLIIDTLINQKIGTLIIGHN |
| | TDWKQKINLGKRNNQNFVSIPYNKFIEMLSYKAEMVGIKVIITEESYT |
| 327 | MYLTTVNRLRLNQNEFNLVKELCWLSKNLYNSTLYEVRQHYFNTSEFLKYTKAYHILKNTENYKLLPSQ |
| | VAQQTMKVVERTMKSFFGLLREKKKGNYNKPIKIPRYLNKEGKFVLLYTPAHMRYISNNQIRLTVKKEL |
| | LEKHNLKELIITIPKHIIGKTIKELRINPLGQFLKVEFIYLNNENNYPKVTKNKNILSIDLGIDNLCTMINNVN |
| | NQPIIIDGREIKSINRLFNKNLSKYKSISKKVNDRYSTKKIDRLYYKRNNVFKDKFHKVSNYIINYCIDNNI |
| | SKVIIGYNQEWKQNINIGKTN |
| 328 | PAKVAQQILMRLHEAWQGFFSSLASYKEEPEKFFCSPRIPSYLHKTNGRFPCIYTIQAISKKYLKHSQIKPS |
| | KTNIVIPTNVNQIRQVRLVPKGSYYVFEVVYKRLEEPQIHTSDGIAGIDIGLNNLAAVTSNIKGFKPILVNG |
| | KPLNKINAYYHKIRSKLQSLLPSKHKTSHQIQNLTRKRNFKIYDYLHKSSRLIIDYLAANQIGTLIIGHNDK |
| | WKQSIGLGKRNNQNFVSIPFDRFISMLKYKAKLIGIKVIITEESYTSKCSFIDEEPLSK |
| 329 | MTRKKAVKVLRKQKKRENMQRFTQKQNIGRACLTAKEFRLLQRMSHSSKALRNVGLYTMKQSYLNH |
| | NRMATVKEVDTAMQADTNYWGVQSNSVQAIRRALFTEVKSFFKALEQWKKNPEKFTGRPKFPNYSRS |
| | TDKRIIEIYQVPKVDENGYWMIPMNVAFRKKFGSIKIRMPKNLRNKNISYIEIVPKQKGRFFEVHYTYEM |
| | HVSQMKKPSTTTSNALSCDLGVDRLLSCVTHTGDAFLIDGKKLKSINQY |
| 330 | MARKKAVKVLRKQKKRENMQRFTQKQNIGRACLTAKEFRLLQRMSHSSKALRNVGLYTMKQSYLND |
| | NKMATVKEVDTAMQADTNYWGIQSNSVQAIRRALYAEVKSFFKALEQWKKNPEKFTGRPKFPNYSRST |
| | DKRIIEIYQVPKVDENGYWIIPMNVAFRKKFGSIQIRMPKNLRNKNISYIEIVPKQKGRFFEVHYTYEMHV |
| | SQMKKPPATTSNALSCDLGVDRLLSCVTNTGDAFLIDGKKLKSINQYFNKMICNLGQKNMDNGISKRIV |
| | TNKMAALWHKRERQINGYIA |
| 331 | MTRKKAVKVLRKQKKRETMQRFTQKQNIGRACLTAKEFRLLQRMSHSSKALRNVGLYTMKQSYLNHN |
| | KMATVKEVDTAMQADMNYWGIQSNSVQAIRRALYAEVKSFFKAMEKWKKNPEKFTGRPKFPNYSRPT |
| | DKRIIEIYQVPKVDDNGYWMIPMSVAFRKKFGSIKIRMPKNVRNKKISYIEIVPKQKGRFFEVHYTYELH |
| | VSQMKKQSTTTSNALSCDLGVDRLVSCATNTGDTFLIEGKKLKSINQYFNKMIRNLQQKNVENGISKRV |
| | VTNKMAALWHKRERQINGYISQTVGLLFKKVKAFGIDTVVVGYNAGWKQKSDMGKKNN |
| 332 | MWFKTGKILSGYDLTAQMKTNKHFNAGYASSMQQTCLNVGEAFKSFKKLLSKAKKGELNQKPLPPKY |
| | RKSGGLFTVTYPKRWLKLKSGLIRFPLGNQVKAWFGISEFFLPLPTNLNWSNIKEIRILPRNGCFYAEFVY |
| | KTSVEPIKLNKSNVLGIDHGLNNWLTCVSNVGTSLVVDGLHLKSLNQWYNKSIAKIKENKPYSFWSKRL |
| | ARITEKRNRQMRDAVNKAARIAVNHCLENNIGTLIFGWNEGQKNSSDMGKKNNQKFVQIPTARLKNRIE |
| | QLCEQYGIEFVETAIFLYF |
| 333 | MARKKAVKVLRKQKKRENMQRFTQKQNIGRACLTAKEFRLLQRMSHSSKALRNVGLYTMKQSYLNN |
| | NKMATVKEVDTAMQADMNYWGMQSNSVQAIRRALFTEVKSFFKAMEQWQKNPEKFTGRPKFPNYSR |
| | STDKRIIEIYQVPKVDDNGYWMIPMSVAFRQKFGSIKIRMPKNLRHKKISYIEIVPKQKGRFFEVHYTYEM |
| | HVSQMKKQSTTTSNALSCDLGVDRLVSCVTNTGDTFLIDGKKLKSINQYFNKMICNLQQKNIENGISKR |
| | VVTNRMAALWHKRERQINGYISQTVGLLFKKVKAFGIDTIVVGYNVG |
| 334 | MNSLRKAVKVLRKQKKRENIQRFTQKQNIGRACLTAKEFRLLQRMSHSSKALRNVGLYTMKQSYLNH |
| | NKMVTVKEVDTAMQADMNYWSMQSNSVQAIRRSLFTEVKSFFKAMEQWKKNPEKFTGRPKFPNYSGS |
| | TDKRIIEIYQVPKVDENGYWMIPMNVAFRKKFGSIKIRMPKNLRNKKISYIEIVPKQKGRFFEVHYTYEM |
| | HVSQMKKPFTTTSNALSCDLGVDRLVSCVTNTGDTFLIDGKKLKSINQYFNKMICNLQQKNIENRISKRV |
| | VTNQMAVLWHKRERQINGYISQTVGLLFKKVKAFGIDTIVVGYNVGWKQKADMGKKNNQTFLQIPFH |
| | KLIAAIENKCVKEGIRFLKQEESYTSKASF |
| 335 | MARKKAVKVLRKQKKRENIQRFTQKQNIGRACLTAKEFRLLQRMSHSSNALRNVGLYTMKQSYLNHN |
| | KMVTVKEVDTAIQADMNYWSMQSNSVQAIRRSLFTEVKSFFKAMEQWKKNPEKFTGRPKFPNYSGSTD |
| | KRIIEIYQVPKVDENGYWMIPMNVAFRKKFGSIKIRMPKNLRNKKISYIEIVPKQKGRFFEVHYTYEMHV |
| | SQMKKPFTTTSNALSCDLGVDRLVSCVTNTGDTFLIDGKKLKSINQYFNKMICNLQQKNIENRISKRVVT |
| | NQMAVLWH |
| 336 | MARKKAMKVLRKQKKRENMQRFTQKQNIGRACLTAKEFRLLQRMSHSSKALRNVGLYTMKQSYLNH |
| | NKMATVKEVDTAMQADMNYWGIQSNSVQAIRRALYAEVKSFFKAWEQWKKKPETFTGRPKFPNYSRS |
| | TDKRIIEIYQVPKVDDNGYWMIPMNVAFRKKFGSIKIRMPKNLRNKKISYIEIVPKQKGRFFEVHYTYEIH |
| | VSQMKKPSTTTSNALSCDLGVDRLLSCVTNTGDTFLIDGKKLKSINQYFNKMIRNLQQKNMDNGISKRI |
| | VTNQMAALWHKRERQINGYISQTVGLLFKKVKACNIDTIVVGYNVG |
| 337 | MARKKAMKVLRKQKKRENMQRFTQKQNIGRACLTAKEFRLLQRMSHSSKALRNVGLYTMKQSYLNH |
| | NKMATVKEVDTAMQADMNYWGIQSNSVQAIRRALYAEVKSFFKAWEQWKKKPETFTGRPKFPNYSRS |
| | TDKRIIEIYQVPKVDDNGYWMIPMNVAFRKKFGSIKIRMPKNLRNKKISYIEIVPKQKGRFFEVHYTYEM |
| | HVSQMKKPSTTTSNALSCDLGVDRLLSCVTNTGDTFLIDGKKLKSINQYFNKMIRNLQQKNMDNGISKR |
| | IVTNQMAALWHKRERQINGYISQTVGLLFKKVKACNIDTIVVGYNVG |
| 338 | MKRSVTVKLQPSKGQETTLFELASVGAKIWNHVNYLRRQQFFQEQIVDFNKTEKIVYEEYKKEIGSATV |
| | QQIARKNAEAWRSFFSLLRKKRNKELPNWFKPRPPNYLKEDGKRKPLIVLRNDQYKIEGNKLILKGLGK |
| | FGKLEIQFKGRIHLKGKQGRLEITYDEVKRKWYAHISFTVEEKLEGEEWVALPRQPKGNLSAGIKSIDFY |
| | WRKGMADYQSKLNKSGAKTSRKLKRMHEKAKLQAKHYINTAVRQTVRKLYELGVSRIVVGYPKGIAR |
| | NSDKGKKQNFLLSHIWRFNYVIKRLTEVAEEYCIQVELVDEAYTSKICPVCGRPHEGARFVRGLFKCPET |
| | GFVFNADLVGAFNILKKKVKTITPNLGGLYAQGRGNWPKARPGGFERTTLTGSLMKTPQTFPPVG |
| 339 | MAMLGRTLKVRLYPDASQATQLLEMSREYQHLANLVSQWVFDHDFPLNSLKINHALYKVFRRESLLNS |
| | QMIQSVFRTVVARYKTVLEQMKHHPYRYQDDDKKWVRVTKDLTWLFKPLHFSRPQADLVRRSNYSFG |
| | NGLTEISLTTLEKRAKMPFTIKGVEHYFQNGWKLGTAKLIHSRGKWYLHIGITKEVADFDSTIPSQIVGID |
| | RGLRFLTTTFDQRGKTRFFDGKKVLLKRHKFQKIRAELQHRGTKSAKKKLKQLQQRENRWMTDINHQL |
| | SKTLVTLYGPQTLFVL |
| 340 | MKGGSDSKSSVKYSKQLNILSQRRESFLRDYFYKCAWYICRYAKAADVDVIVMGHNDGQKQEIDLTDN |
| | VNQNFVSIPYTKFITILKTVASKCGIAVVIREESYTSQASLLDMDDIPTYKKGENKKHAFSGKRIHRGLYR |
| | SKNGTLLNADINGAANILRKEYPNAFDSIKNFAYLYVTTISIGYKDLYRNAKACAGRPKSYKYHKSGRCT |
| | VVRHMERSHKKCEYCKLWGKGKFVWRPDKNKQDTQQGKAA |
| 341 | MKQQVSFKFRLKPDGQQERQMRRFAGACRFVFNRALALQNENHEAGKKYIPYTKMASWLVEWKKDT |
| | ETEWLKDSPSQPLQQSLKDLERAYKNFFQNRAAFPRFKKRGQNDAFRYPQGVKLDQENSRIFLPKLGW |
| | MRYRNSRQVTGVVKNVTVSQSCGKWYISIQTEREVSTPVHPSASMVGLDAGVAKLATLSDGTVFEPVN |
| | SFQKNQKKLARLQRQLSRKVKFSNNWQKQKRKIQRLHSRIANIR |
| 342 | MHDDLAFHQFCSVYTGPVVIKKQAFKFLLEPNKGQLSDFLAFAGSCRYVYNKGLALLNENYRSGKKFIG |
| | YNQLASELVEWKNEESLSWLKEAPSQCLQQSLRDLDRAFRNFFTGKSQYPKFKKKGRHDSFRIPCQRVR |
| | VDQEKKLVSLPKVGWVKYRKSREIIGDLKNATISLNQGEWYISFNTEQTVPDPIHPSDIKTTIVLNNVNSV |
| | HLSSGVGGDNTYQAEEKKKLIRLNKTLTRRKKYSKNWLKTKAKIDRVRSKAARIRLDNIHKATTAICKN |
| | HAVVEVVNLMDSVSAKNDNTLSMRYEFVRQLIYKQEWLGGEIIRRESKLL |
| 343 | MTKENPSNYKTLQIWIKKGHRMYSYFQECCHNAKNMYNTTNFYIRQVYTGLTQEKELQPLQKEVLANI |
| | HKNIGKMNDTQLLAYQKKLEKEKLKPKEEQKEIKCNLFSEPNFEKPYVDYNFLDALFKAMIQNDYRALP |
| | TQCSQSIMKGLFQNWKSFFASLKDYKKNPNKYVGMPRIPKYIRSSEKEILYTNQDCIIKNSRFLKFPKTKL |
| | QLNIGKLGFTEGKLKQVRVIPKYNEYVVELVIDIPSEQQIIEENARYMSIDLGIDNLATIVTNTGMKPVLV |
| | KGKHVKSINQYYNKMKSHFTSILRNGKQTNEGPLTSKRIENLHQKCYLKIKDVFHKVSHHIVKLAQEEE |
| | VCKIVIGQNKSWKQETNMGKRNNQSFCHIPHNLLVQMITYKANAVGIQVVVTEESYTSKASFLDNDFIP |
| | TYGEN |
| 344 | MFAGSCRFVYNKALALLNDNYHSGKKFMGYNQLATELVEWKSEESLSWLKASPSQCLQQSLRDLDRA |
| | FRNFFSGKAQYPKFKKKGRHDSFRIPCQRVRVDQDKKMVSLPKVGWVKYRKSREIIGELKNVTISMKQD |
| | KWYISFNTESMVPDPMHPSDIKTKIVLSDQCEFPIRLDSSMDSSHQLDEVKKLARLNRILIRRIKYSSNWL |
| | KTKGKIDRIKARLARCRLDNIHKVTTAICKKHAVVEVLSLMDSVSDKNDITLSMRYEFVRQLIYKQEWL |
| | GGEVIRRELA |
| 345 | MKQQVSFKFRLKPDGQQERQMRRFAGACRFVFNRALALQNENHEAGKKYIPYTKMASWLVEWKKDT |
| | ETEWLKDSPSQPLQQSLKDLERAYKNFFQNRAAFPRFKKRGQNDAFRYPQGVKLDQENSRIFLPKLGW |
| | MRYRNSRQVTGIVKNVTVSQSCGKWYISIQTEREVSTPVHPSASMVGLDAGVAKLATLSDGTVFEPVNS |
| | FQKNQKKLARLQRQLSRKVKFSNNWQKQKRKIQRLH |
| 346 | PLYGYAQQLTAMANNLSNAARFRQRQLMTAAKKEPADWTANEQEVMDELYAAFPEDFMDGPADRQG |
| | FLNVYGNLEKLMRRSGNPDYFAEGFPRQCSQQVLKQAARDMKAFFDSLKAYQKNPSAFTGKPQLPGYK |
| | RKGGHCTVTVTNQDCKVSEKDGMWYAAFPLRKDCPLAIGHPIPDAVLKEATITPDNGRYRFCLKFEVM |
| | AELPPVTEQPGRVCAIDFGVDNLMAVTNNCGLPCLIYKGGVAKSINQKYNKTVAVMVSRQTAATGKKY |
| | VPDAAYHAVTNRRNDRIGDLLHKCAKHFITWCVENRIDAIVLGVNKYWKQEVALGDDRNQNFVQIPFL |
| | VLRRIIGYLAEWNGIHCIEQEESYTSKASFPDMDA |
| 347 | MEKAYSYRFYPTPEQESLLRRTLGCVRLVYNQALHERTQAWYERQERVGYSQTSSMLTNWKKQEDLD |
| | FLNQVSCVPLQQGLRHLQTAFTNFFAGRAKYPNFKKKHQGGSAEFTKSAFKFKNGQIYLAKCLEPLAYK |
| | CRWYGRNYIEIDRWFPSSKRCSSCGHIVEKMPLNIREWDCPNCGTHHDRDLNASKNILAAGLAVSDCGA |
| | SVRPEQSKSVKATAKKQKPKL |
| 348 | MRTACKCRASPTPAQATQLGRTFGCVLLVWNKTLAERHAAYHQRGEKTPYGQTGRALTGWKKTTDLA |
| | FLSEVSSVPLQQTLRHQHTAFQNFFSGRARSPRFKSRSSRQSAHYTRSAFRVRDGRLTLGEFRRRLEYKA |
| | ARRGRTLAVADRWFPSAKTCAHCGHLLDTLPLGTRFWACPRRRARHDRDVNAAKHILAAGRAAVRAR |
| | AGDACGAGVRRQGPSLPRSATKQEAAAARQST |
| 349 | MYCTVKQQLKHLSKEEYLLLKELCHTAKNLYNEGLYQVRQHYFQEKNYLNYQNNYHLLKGSENYKRL |
| | NSNMAQQILKEIDGVWKSFFGLIRLAKQGKYDFRAIRIPWYLPKDQQFALVVKRNRRVHDYLSKTCRKII |
| | NYCLNHRIGTLVIGYNENLQKGSNLGRRNNQNFVNIPIGMIKKKLEYLCQLYGMTFVQQEESYTSQASF |
| | WDRDELPTYDPSNVKTYTFSGKRVKRGLYRTASGKLLHADIHDALNILRKSNVVALTGLYARGEVDTP |
| | VRIRIA |
| 350 | MEKAYSYRFYPTPEQESLLRRTLGCVRLVYNQALHERTQAWYERQERVGYSQTSSMLTNWKKQEDLD |
| | FLNQVSCVPLQQGLRHLQTAFTNFFAGRAKYPNFKKKHQGGSAEFTKSAFKFKNGQIYLAKCLEPLAYK |
| | CRWYGRNYIEIDRWFPSSKRCSNCGHIVEKMPLNIREWDCPNCGTHHDRDLNASKNILAAGLAVSDCGA |
| | SVRPEQSKSVKATDKKQKPKL |
| 351 | MRLVQKHLINKNHPYWSYFDQQAFLSKNLFNLANYHIRQHFFNTRTVLSFTSLYHLVSKTDAYGALPNT |
| | KVAKQIIRRVHKAWIGYKQAHKDWQRHPEKYLGEPKIPKYKYKQNGRYIVVFPDETVSKPALRKGVVK |
| | LTPCPIEFNSGLRQVNEVRVIPRSGCYVVEIVYEQDRVASTTGDATAGVDIGLVNLVTLTTNQSGVKPLLI |
| | KGGALKAINTYYNKQKAKIQSELATKYQRKSSRRLESLTFKRNCRVDNYLHTVSLYSD |
| 352 | MRTAYKCRAHPNPEQAAALSRTFGCVRLVWNKTLNDRNRRYKTENKGTSYRETDATLTIWKRSDVLG |
| | FLSEVSCVPLQQTLRHQHSAFQNFFSRRSRYPRFKSRTGRQSAHCTRSAFRMRGGSLTLAKMSTPLPFTW |
| | SFTGVDVGELNPATVIVSREPDGRWYVSFAVDVRDSAAARPANREIGLDLGLRNFVTTSDGTRVPRPRS |
| | MDRKARNLGCRTRHDRDLNAAKNILAAGRAVARGFSGDACGADVRRQGPSLPLSAVNQEAHAETLGS |
| 353 | MLTGFRYRLAPTGEQAGLCQVYGDICRAVWNTGLHQRREAVRRWQRGQDLPFCGYHLQARQLAEAK |
| | TEEEWLKAAPSHILQQTLRDLDRACRDHGTFNVRWRAKGRWKPSFRFPAGARVIVQRLGRKWGRLKLP |
| | KLGWVRFRWSRSAKGTVDAPGTHVRQKAGLNRAILARGWHGFKLACQNAARRSATRIVEVNPAYTSQ |
| | TCHPCGHVASENRESPSVFRCGACGYRAHADVNAARNTRARGWTSPSG |
| 354 | MARKKAVKVLRKQKKRENIQRFTQKQNIGRACLTAKEFRLLQSMSHSSKALRNVGLYTIKQSYLNNNK |
| | MATVKEVDTAMQADMNYWGIQSNSVQAIRRSLFTEVKSFFKGLEQWKKKPETFAGRPKFPNYSGSTDK |
| | RIIEIYQVPKVDDNGYWMIPMNVAFRKKFGSIKIRMPKNVRNKKISYIEIVPKQKGRFFEVHYTYEMHVS |
| | QMKKPSTTTSNALSCDLGVDRLLSCVTNTGDTFLIDGKKLKSINQYFNKMIRNLQLK |
| 355 | MKNSKKNEEEDNWGYRRYSIVVRKSSPDYQKIDELCFKSKNLFNATLYSQRQSYFDTGKFIKHNDLNTS |
| | FAHTNQPDYRALPAKVSKYTQKKVDQAIKSFLGLKKSKKITFTPKIPKYLKKDGRFVTEYEKDALSFKRE |
| | GFIKLSKTNIYIPIPNKLKIKGKGKDLKKVFRVVRLVPKTGYYLIEVLYKKSIPKKRKKKMTHKTRFASID |
| | LGVNNLVTVTSNVFQPLIINGRPIKSINQYYNKYRKRKQQLLPKNQYTSKAIRQLGYKREMKLNDYLHK |
| | SAAFLVNYLVSQTIDVLVIGTNKGWKQNINIGKRNN |
| 356 | MTLTERHIIRPTHPIFKRIKDFCHLSKNLYNYANFILREHYFAGFKLPTAYDLINRFVKESQRDYKALPAQ |
| | SAQQVLMLLSQNWKSYLKALKAYKLKPSSFLARPKIPKFKPKDGVSIGVLTNQQTSFTKGRMTKIKFPK |
| | KANLKRLITKINPQTSRLKQVRLIPKTTCFIVEVVYEQTTHKLPQTHGIGIMGIDLGLNNFVTAIDNQSSPFI |
| | IKGGGVKSVNQWFNKLKAHYQAKAKTSNKRFWTKRLGKLALWRECKVNDFMHKASAYVVGHCLKK |
| | GISTIVIGKNDGWKQELKLGKRTNQNFTNIPYESFIEKLAYKCALVGITLHTTEERFTSKCDHLANEPMQH |
| | HEQYLGKRVK |
| 357 | MSKKTKKKVKNLGCQQVLLHPDQELRAILEYLCGEANKVFNCSVYYARQVWFKENRFVSKSELCEQM |
| | KWNRHFNAMYASSAQQICNGVVESFSSFRQLLKLFGKGELANKPKPPNYRKPGLFTVSYPKRWLKFTN |
| | EGIRVPLGRKVKAWFGLEAFYIPMVSNLDWDSIKEIRILPRHGCFYTEFVYEMKTPVAVKLDAGQALSID |
| | HGLDNWLTCVDTQGDSFIIDGKHLKSKNQWYNKQIATIKENQPQGFWSQRLARMTEKRNRQMRDAVN |
| | TRSAISY |
| 358 | MTLTERHIIRPTHPIFKRIKDFCHLSKNLYNYANFILREHYFAGFKLPTAYDLINRFVKESQRDYKALPAQ |
| | SAQQVLMLLSQNWKSYLKALKAYKLKPSSFLARPKIPKFKPKDGVSIGVLTNQQTSFTKGRMTKIKFPK |
| | KANLKRLITKINPQTSRLKQVRLIPKTTCFIVEVVYEQTTHKLPQTHGIGIMGIDLGLNNFVTAIDNQSSPFI |
| | IKGGGVKSVNQWFNKLKAHYQAKAKTSNKRFWTKRLGKLALWRECKVNDFMHKASAYVVGHCLKK |
| | GISTIVIGKNDGWKQELKLGKRTNQNFTNIPYESFIEKLAYKCALVGITLHKTEERFTSKCDHLANEPM |
| 359 | MGTAYKCRAYPDPEQAAIFGRTFGCVRLVWNKTLAERHRAWHSHGRRTSYKETDAALTAWKKTEELA |
| | FLSEVSSVPLQQALRHQHAAFAGFFAGRARYPRFKTRTSRQSAHYTRSAFRMRDGELQMAKAISDCGW |
| | GEFRRQLEYKAHRAGRTLIVIDRWYPSSKTCSNCGHLLEKLSPSTRHWTCPGCRTRHDRDHNAAKNILA |
| | AGRAAAGARPGEVCGADVRRQGSPLPQSATKQKPPRREPRESPSFQGEEEVNHALWRGGRPPR |
| 360 | MARKKAVKVLRKQKKRETMQRFTQKQNIGRACLTAKEFRLLQRMSHSSKALRNVGLYTMKQGYLND |
| | NKMATVKEVDTAMQADMNYWGIQSNSVQAIRRALFTEVKSFFKALEQWKKNPEKFTGRPKFPNYSRST |
| | DKRIIEIYQVPKVDDNGYWTIPMNVAFRKKFGSIKIRMPKNLRNKKISYIEIVPKQKGRFFEVHYTYEMH |
| | VSQMKKPSTTTSNALSCDLGVDRLLSCVTNTGDTFLIDGKKLKSINQYFNKMIRNLQQKNMDNGISKRI |
| | VTNKMAALWHKRERQINGYISQTVGLLFKKVKAFDIDTIVVGYNMGWKQKSDMGKKNNQRFVQIPFH |
| | KLMAAIENKCVKEGIRFLKQEESYTSKASFLDKDPVPVW |
| 361 | MARKKAVKVLRKQKKRENIQRFTQKQNIGRACLTAKEFRLLQRMSHSSKALRNVGLYTMKQSYLNHS |
| | KMATVKEVDTAMQADMNYSGMQSNSVQAIRRALYAEVKSFFKALEQWKKKPEAFTGRPRFPNYSRST |
| | DKRIIEIYQVPKVDDNGYWMIPMNVAFRKKYGSIKIRMPRNIRNKKISYIEIVPKQKGRFFEVHYTYEMH |
| | VSQMKKQFTTTSNALSCDLGVDRLLSCVTNTGDAFLIDGKKLKSINQYFNKMIRNLQLENIENGLSKRV |
| | VTNEMAALWHKRERQISGYISQTVGLLFKKVKAFDIDTIVVGYNTGWKQKSDMGKKNNQKFVQIPFHK |
| | LIAAIENKC |
| 362 | MYLCIKQQLKKISKEDYENLRELSHVAKNLYNYGLYNVRQYYFEQKEYLNYEKNYAIYKNNENYKLLN |
| | SNMAQQVLKEVDGVFKSFFGLIKLAKKGKYNFRDIKLPKYLKKDGFATLVIGFVRIKGITKKQSILRNNR |
| | NNRVNDYINKTCRYIINYCLDNNIGNLVIGYNETLQRDSNLGKVNNQNFVNIPVGNIKEKLEYLCKLYGI |
| | NFVKQEESYTSKASFFDNDNIPKYNADNPIQATFSGKRIKRGLYKTKSGYAYKNKDCINF |
| 363 | MTKTKKLMGVQQCLINPDKDLKAILEYICSESNKLHNCAVYYARQIWFKTRRFVTGFDLVNELGSNKHF |
| | STLPSEAAVQTCLSVGESVKSFSELLKKSRKGELEQNPKFPKYRKQGYQLVAFPKRALRLVGNTIRFPLG |
| | LQVKAWFGLKEFFLPMPSNLDFGLLKEVRILPRNGAFYAEFVYPKANIKAELDPAKCLGIDHGLNNWLT |
| | CVSNVETSFIVDGLHLKSLNQWYNKHTSDLMEGKPNGYWTKRLANREHPKFALSRNT |
| 364 | MARKKAVKVLRKQKKRENMQRFTQKQNIGRACLTAKEFRLLQRMSHNSKALRNVGLYTIKQSYLNDN |
| | KMATIKEVDTAMQADTNYWGMQSNSVQAIRRTLFTEVKSFFKALEQWKKNPEKFTGRPKFPNYSRSTD |
| | KRIVEIYQVPKVDDNGYWMIPMNVAFRKKFGSIKIRMPKNLKNKKISYIEIVPKQKGRFFEVHYTYEMH |
| | VSQMKKPSTTTSNALSCNLGVDRLVSCVTNTGDAFLIDGKKLKSINQYFNKMICNLGQKNMDNGISKRI |
| | VTNKMAALWHKRERQINGYIAQTVGLLFKKVKEFDIDTIVIGYNAGWKQNSHMGKKNNQKFVQIPFQK |
| | LMAAIENKCIKEGIRFFKQEESYTSKASFIDKDPVPVWSKDDKTQYCFSGKRITRGLYQSKAGTCIHADIN |
| | GALNTLQKSRVVQLDDNLKVKTPILLEVQKRKAVASRIA |
| 365 | MLKGGRVQLVERHVIKKSHKYHQEIDNLCFLSKNLYNVANYLIRQKLFQSGEILNYNQVQKLFSGSVDY |
| | KAIPAKVSQQILMVLDKNWKAFQAASKSYLKNPSKFLGKPKLPKYKHKTDGRNLLIYTVQALSKPALA |
| | KGFVNPSQTNIFIPTNAKDIAQVRIVPKLDHYVVEVVYHKEIEEKQLETTRIASVDLGLNNLAAVTFNQA |
| | GLVPFLINGRPLKSINQFFNKKKAELQAILKTGTSKRLKKLCTKRNLKVDDYLHKASRYLINKLVELNIGI |
| | LVIGKNDNWKQKIAIGNRNNQNFVQVPHTRFIDQITYKAELTGIKVIVNEESYTSIASFWDQDEIPVVRSV |
| | DSKTVKAGLLDFYEIESQCPQVGISHNFKFI |
| 366 | MARKKAVKVLRKQKKRENMQRFTQKQNIGRACLTAKEFRLLQRMSHSSKALRNVGLYTMKQSYLNN |
| | NRMATVKEVDTAMQNDMNYSGIQSNSVQAIRRSLFTEVKSFFKALEQWKKNPEKFTGRPKFPNYSRST |
| | DKRIIEIYQVPKVDEKGYWMIPMNVAFRKKFGSIKIRMPKNLRNKNISYIEIVPKQKGRFFEVHYTYEMH |
| | VSQMKKQPTTTSNALSCDLGVDRLVSCVTNTGDTFLIDGKKLKSINQYFNKMICNLQQKNMDNGISKRI |
| | VTNTMAALWHKRERQINGYIAQTVGLLFKKVKEFNIDTIIVGYNAG |
| 367 | MARKKAVKVLRKQKKRETMQRFTQKQNIGRACLTAKEFRLLQRMSHSSKALRNVGLYTIKQSYLNDN |
| | KMATVKEVDTAMRADMNYSGMQSNSVQAIRRALFTEVKSFFKAMEQWKKNPEKFTGRPKFPNYSRST |
| | DKRIIEIYQVPKVDKNGYWIIPMNVAFRKKFGSIQIRMPKNVRNKKISYIEIVPKQKGRFFEVHYTYEMHV |
| | SQMKKQSTTTSNALSCDLGVDRLVSCVTNTGDTFLIDGKKLKSINQYFNKMICNLQQKNMDNGLSKRIV |
| | TNRMAALWHKRERQINGYISQTVGLLFKKVKEFDIDTIVVGYNTGWKQKSHMRKKNNQTFAQIPFHKLI |
| | VAIENKCLKEGIRFL |
| 368 | MLTGFRYRLSLTDEQAERCAEYGDICRAVWNTALDQRRQAVQRWQRGYDQLFCGYHLQATQLAETKT |
| | EETWLRAAPSHILQQTLKDLDRACRDHGTFGVRWRGKGRWKPSFRFPDPKQITVERLGRRWGHLKLPK |
| | LGWVRFRWSRAPKGAVRSATVSRDGEHWYVSLLCEDGEHTPGEHAVPDAAVGIDRGVAVAVATSDGD |
| | LFDRTLQTPKEHERERRLRRKFKLACLNAARRTGTRIVEVDPAYTSQTCNPCGHVAPENRESQSVFRCTS |
| | CGHTAHADVNAAQNTLSRGWTGSLSG |
| 369 | MIRRQAYKFQLKPNPEQIASMKSFAGACRFVYNRALTMQSDVWRNGDRYIPYNKMAPWLVEWKSQEE |
| | MSWLSNAPSQILQQSLKDLDKAFNNLFARRATFPSSKKKGKNDAFRYPTQRVKLDEANERIQLPKLGW |
| | VRYRKSRNITGVIKNVTVSMKLDKWYVSLQTESEVETPAPLQSSIIGLDTCNIECLTTSEGTDFLSQATLP |
| | KMEKSLEKSIRRLRRKKKFSCNWVKQRHKVNRLLHRISNMRKDHFHKISTVLSKNHAIVVIENLEHATS |
| | LPNRSLGKKAAAVYNIYELKRQLDYKLSWNGGQLVTVHEHDNNLKQVEADSACTAYGAIRAKKILAA |
| | GHAVIACGGVDMLRHPLKQEPSEDNGSTAILL |
| 370 | MIRRQAYKFQLKPNPEQIASMKSFAGACRFVYNRALTMQSDIWRNGDRYIPYNKMAPWLVEWKSQEE |
| | MSWLSNAPSQILQQTLKDLDKAFNNLFSRRATFPSPKKKGKNDAFRYPTQRVKLDEGNERIQLPKLGWV |
| | RYRKSRSITGVIKNVTVSMKLDKWYVSLQTEAEVDEPSSQQSSMIGLDASNIECITTSDSTDFLSQASLPK |
| | MEKSLEKNIKRLRKKKRFSSNWVKQRHKVNRLLNRISNMRKDHFHKISTTLSKNHAIVVIENLEAATSLS |
| | KRPKSTKRFLSNETYELKRQLDYKLNWNGGELMTVRERDNNFKPVSDNASSNLYGRLRAEKILAAGHA |
| | VIACGGAEFLGHPMKQEPSEDGKSTVILL |
| 371 | MIRRQAYKFQLKPTPEQIASMKSFAGACRFVYNRALTMQSDIWRNGDRYIPYNKMAPWLVEWKSQEE |
| | MSWLSNAPSQILQQSLKDLDKAFNNLFARRATFPSPKKKGKNDAFRYPTQRVKLDEGNERIQLPKLGW |
| | VRYRKSRSITGVVKNVTVSMKLDKWYVSLQTEAEVDEPSSQQSSMIGLDTSNIECITTSDSTDFLSQASLP |
| | KMEKSLEKNIKRLRKKKRFSSNWVKQRHKVNRLLNRISNMRKDHFHKISTTLSKNHAIVVIENLEAATS |
| | LSKRPKSTKRFLSNETYELTRQLDYKLIWNGGELVTVRERDNNFKPVSDDASSNHYGKLRAERILAAGH |
| | AVIACGGAELLGHPMKQEPSEDGKSTANLL |
| 372 | MIRRKAYKFQLKPNPEQIASMKSFAGACRFVYNRALTMQSDIWRNGDRYIPYNKMAPWLVEWKSQEE |
| | MSWLSNAPSQILQQSLKDLDKAFNNLFARRATFPSPKKKGRNDAFRYPTQRVKLDEGNERIQLPKLGW |
| | MRYRKSRSITGVIKNVTVSMKLDKWYVSLQTEAEVDDPSPKQSSIIGLDTSNIKCITTSDSIDFLSQASLPK |
| | MEKSLEKSLKLLRKKKRFSSNWAKQKHKVNRLLHRISNMRKDHFHKISTALSKNHAIVVIENLEDATSL |
| | SNHRKRTSGFTLNDIYELKRQLDYKLKWNGGELVTVCERDDNLKPIIDDASSNHYGRLRAEKILAAGHA |
| | VIACGGADLLGHPMKQEPSEDGKSTVILL |
| 373 | MIKKQAFKFLLEPNKGQLSDFLAFAGSCRFVYNKGLALLNENYRAGKKFIGYNKLASELVEWKNEESLS |
| | WLKEAPSQCLQQSLRDLDRAFRNFFTGKSQYPKFKKKGRHDSFRIPCQRVRVDQEKKLVSLPKVGWVK |
| | YRKSREIIGDLKNATISLNQGKWYISFNTEQTVPEPIHPSDIKTTIVLNNVNSVHLSSGVGGDNTYQAEEK |
| | KKLVRLNKTLTRRKRYSKNWLKTKGKIDRVKSKAARIRLDNIHKATTAICKNHAVVEVVNLMDSVFDK |
| | NDNTLSMRYEFVRQLIYKQEWLGGEIIRRESKLL |
| 374 | MIKKKAFKFLLEPSKSQISDVLVFAGACRFVYNKGLALLSENYNNGKPFLNYNKLAPLLVEWKSDHKFE |
| | WLKLCPSQCLQQSLRDLDRAFQNFFSGRALYPRFKKKGRSDSFRVPCQRVRLNQEKGLVSLPKLGWVK |
| | YRKSREVTGNLKNVTISKKLDKWYISFNTEEFVSEPVHPSINKTKVLLNDGYVTLCAGNEVSVESFTGIV |
| | DEKKIKRLNKELSRKVKHSNNWLKSKIKIYIIRTSSGNFRLDALHKITTAICKKHAVVEVVDVKNFVSDK |
| | NNIAKNMRYEFVRQLLYKQEWLGGKIVQLDA |
| 375 | MIKQQAFKFALKLNDQQKANMLLFAGACRFVYNKGLALLKESYESGQKHMHYNQLAPLLVEWKSDPA |
| | LSWLKQAPSQSLQQSLRDLDKAFSNFFYGKAEHPRFKKKGQHDAFRFPSQRVKVDQEKQLVLLPKLGW |
| | VKYRKSRNITGAIKNVSISGKLGNWYISFNTQTDIAEPIHPAISKIGVYVDTKKNITLSDGTQYIPPQSLITL |
| | PKQIQRLTNCLRKKNRYSNNWLKSKHRINRLSSRLNQVKVDYLHKASTAISKNHAMIVIADFEKKSFSA |
| | DKQQKNLTTCEKSTSIHYELIRQLTYKQEWHGGLVIKLSAEKNVDAESAWTKACNLLAAGLAVTACGG |
| | EVSKDSPMKQEP |
| 376 | MKRLQAFKFQLRPGGQQEREMRRFAGACRFVFNRALALQNENHEAGNKYIPYPRMASWLVEWKNATE |
| | TQWLKDAPSQPLQQSLKDLERAYKNFFQKRAAFPRFKKRGQNDAFRYPQGVKLDQESSRIFLPKLGWM |
| | RYRNSRQVTGVVKNVTVSQSCGKWYISIQTESEVSTPVHPSVSMVGLDAGVAKLATLSDGTIFDPVNSF |
| | QKNQKTLARLQRQLSRKVRFSNNWQKQKRKIQQLHSRIANIRRDYLHKVTTTVSKNHAMIVIEDLKVSN |
| | MSRSAAGTVSQPGRNVRAKSGLNRSILDQGWYEMRRQLEYKQLWRGGQVLAVPPAY |
| 377 | MKRLQAFKFQLRPGDQQECEMRRFAGACRFVFNRALALQNENHEAGNKYIPYGKMASWLVEWKNAT |
| | ETQWLKDSPSQPLQQSLKDLERAYKNFFRKRAAFPRFKKRGQNDAFRYPQGVKLDQENSRIFLPKLGW |
| | MRYRNSRQVTGVVKNVTVSQSCGKWYISIQTESEVSTPVHPSASMVGLDAGVAKLATLSDGTVFEPVN |
| | SFQKNQKTLARLQRQLSRKVKFSNNWQKQKRKIQRLHSRIANIRRDYLHKVTTTVSKNHAMIVIEDLKV |
| | SNMSKSAAGTV |
| 378 | MYYDQKKAFKFLLEPSKSQISDVLVFAGACRFVYNKGLALLSENYNNGKPFLNYNKLAPLLVEWKNDN |
| | KLEWLKFCPSQCLQQSLRDLDRAFQNFFSGRSQYPRFKKKGRSDSFRVPCQRVRLDQEKGLVSLPKLGW |
| | VKYRKSRAITGDLKNVTVSRKFDKWYISFNTEEVVSNPVHPSVDKTRILLNDGYVTLCTGGDLSVKKFT |
| | SLVDEKKIKRINKELSRKVKNSNNWLKNKKKIDKIRLKSGSFRLDAIHKITTTICKKHAVVEVVNVKNFV |
| | SDKNNIATSMRYELVRQLLYKQEWLGGKIIHLDA |
| 379 | MNDFLVFAGSCRFVYNKGLALINENYDSGKKFLNYNQLASELVNWKNEECLAWLKMAPSQCLQQSLR |
| | DLDRAFKNFFSGKSQYPRFKKKGRNDSFRVPCQRVRLDQEKHLVSLPKLGWVKYRKSREITGVLKNVTI |
| | SRKLDKWYISFNTEEVVPEPLHPSFSKTKILLNNEWLMQLTACESLVEQFANMEGNKKLRNLNNILGRK |
| | VKYSSNWLKTKKKIDGVKARSSRRRLDALHKITTAICKKHAIVELVNLTDSLPDKNNGSVSMTYEFVRQ |
| | LMYKQEWLGGKVIRLGD |
| 380 | MGSIVIKKQAFKFLLEPNKNHINEFLVFAGSCRFVYNKGLALINENYDSGKKFLNYNQLASELVNWKNE |
| | ECLAWLKMAPSQCLQQSLRDLDKAFKNFFSGKSQYPRFKKKGRNDSFRVPCQRVRLDQEKHLVSLPKL |
| | GWVKYRKSREITGVLKNVTISRKLDKWYISFNTEVVVPEPVHPSFSKAKVLLNNECIVQLTSNESLVEQF |
| | TSMEGNKKLRNLNNILGRKVKYSSNWLKTKKKIDSVKARSSRRRLDALHKITTAICKKHAIVELVNLTD |
| | SLPDKNNGFVSMGYEFVRQLMYKQEWLGGQVIRLGD |
| 381 | MRRFAGACRFVFNRALALQNENHEAGNKYIPYGKMASWLVEWKNATETQWLKDSPSQPLQQSLKDLE |
| | RAYKNFFRKRAAFPRFKKRGQNDAFRYPQGVKLDQENSRIFLPKLGWMRYRNSRQVTGVVKNVTVSQ |
| | SCGKWYISIQTESEVSTPVHPSASMVGLDAGVAKLATLSDGTVFEPVNSFQKNQKTLARLQRQLSRKVK |
| | FSNNWQKQKRKIQRLHSRIANIRRDYLHKVTTTVSKNHAMIVIEDLKVSNMSKSAAGTVSASRGAMSG |
| | QNQV |
| 382 | MKRLQAFKFQLRPGDQQECEMRRFAGACRFVFNRALALQNENHEAGNKYIPYGKMASWLVEWKNAT |
| | ETQWLKDSPSQPLQQSLKDLERAYKNFFRKRAAFPRFKKRGQNDAFRYPQGVKLDQENSRIFLPKLGW |
| | MRYRNSRQVTGVVKNVTVSQSCGKWYISIQTESEVSTPVHPSASMVGLDAGVAKLATLSDGTVFEPVN |
| | SFQKNQKTLARLQRQLSRRVKFSNNWQKQKRKIQRLHSRIANIRRDYLHKVTTTVSKNHAMIVIEDLKV |
| | SNMSKSAAGTVSQPGRNVRAKSGL |
| 383 | MKRLQAFKFQLRPGDQQECEMRRFAGACRFVFNRALALQNENHEAGNKYIPYGKMASWLVEWKNAT |
| | ETQWLKDSPSQPLQQSLKDLERAYKNFFRKRAAFPRFKKRGQNDAFRYPQGVKLDQENSRIFLPKLGW |
| | MRYRNSRQVTGVVKNVTVSQSCGKWYISIQTESEVSTPVHPSASMVGLDAGVAKLATLSDGTVFEPVN |
| | SFQKNQKTLARLQRQLSRKVKFSNNWQKQKRKIQRLHSRIANIRRDYLHKVTTTVSKNHAMIVIEDLKV |
| | SNMSKSAAGTVSQ |
| 384 | MKRLQAFKFQLRPGGQQECEMRRFAGACRFVFNRALALQNENHEAGNKYIPYGKMASWLVEWKNAT |
| | ETQWLKDAPSQPLQQSLKDLERAYKNFFQKRAAFPRFKKRGQNDAFRYPQGVKLDQENSRIFLPKLGW |
| | MRYRNSRQVTGVVKNVTVSQSCGKWYISIQTESEVSTPVHPSASMVGLDAGVAKLATLSDGTVFEPVN |
| | SFQKNQKTLARLQRQLSRKVKFSNNWQKQKCKIQRLHSRIANIRRDYLHKVTTTVSKNHAMIVIEDLKV |
| | SNMSKSAAGTVSQPGRNVRAKSGLNR |
| 385 | MIKKQAFKFLLEPNKGQLSDFLAFAGSCRFVYNKGLALLNENYRAGKKFIGYNKLASELVEWKNEERLS |
| | WLKEAPSQCLQQSLRDLDRAFRNFFTGKSQYPKFKKKGRHDSFRIPCQRVRIDQEKKLVSLPKVGWVKY |
| | RKSREIIGDLKNATISLNQGKWYISFNTEQTVPEPIHPSNIKTTIILNNVNSVHLSSGVGGDNTYQAEEKKK |
| | LVRLNKTLTRRKRYSKNWLKTKGKIDRVKSKAARIRLDNIHKATTAICKNHAVVEVVNLMDSVFDKND |
| | NTLSMRYEFVRQLIYKQEWLGGEIIRRESKLL |
| 386 | MLEPNKGQLSDFLAFAGSCRFVYNKGLALLNENYRSGKKFIGYIQLASELVEWKNEESLSWLKEAPSQC |
| | LQQSLRDLDRAFRNFFTGKSQYPKFKKKGRHDSFRIPCQRVRVDQEKKLVSLPKVGWVKYRKSREIIGD |
| | LKNATISLNQGKWYISFNTEQTVPDPIHPSDIKSTIVLNNVDSVHLSSGGGGDNTYQAEEKKKLIRLNKTL |
| | TRRKKHSQNWLKTKGKIDRVKSKAARIRLDNIHKATTAICKNHAVVEVVNLMDSVSDKNDNTLSMRYE |
| | FVRQLIYKQEWLGGEVIRRESKPL |
| 387 | MGGLPFHFVYAGPAVIKKQAFKFLLEPNKGQLSDFLAFAGSCRFVYNKGLALLNENYRSGKKFIGYNQL |
| | ASELVEWKNEESLSWLKEAPSQCLQQSLRDLDKAFRNFFTGKSQYPKFKKKGRHDSFRTPSQRVRVDQE |
| | KKLVSLPKVGWVKYRKSREIIAELKNVTISMKQGKWYISFNTEHTVPDPIHPSDIKTKIVLNNVNSVHLSS |
| | GIGGDNTSQAEEKKKLIRLNKRLARRKKHSKNWLKTKGKIDRVKSKAARLRLDNIHKATTAICKSHAVI |
| | EVVNLMGSVSDKNDNTLSMRYEFVRQLIYKQEWLGGEVIRRESKPL |
| 388 | MKRLQAFKFQLRPGGQQEREMRRFAGACRFVFNRALALQNENHEAGNKYIPYGKMASWLVEWKNAT |
| | ETQWLKDAPSQPLQQSLKDLERAYKNFFRKRAAFPRFKKRGQNDAFRYPQGVKLDQENSRIFLPKLGW |
| | MRYRNSRQVTGVVKNVTASQSCGKWYISIQTENEVSTPVHPSALMVGLDAGVAKLATLSDGTVFGPVN |
| | SFQKNQKTLARLQRQLSRKVKFSNNWQKQKRKIQRLHSCIANICRDYLHKVTTTVSKNHAMIVIEDLKV |
| | SNMSKSAAGTVSQPGRNVRAKSGLNRSILDQGWYEMRRQLEYKQLWRGGQVLAVPPAYTSQRCACCG |
| | HTAKENRLSQSKFRCQACGYT |
| 389 | MQDDLAFHQFCSVYTGPVVIKKQAFKFLLEPNKGQLSDFLAFAGSCRYVYNKGLALLNENYRSGKKFIG |
| | YNQLASELVEWKNEESLSWLKEAPSQCLQQSLRDLDRAFRNFFTGKSQYPKFKKKGRHDSFRIPCQRVR |
| | VDQEKKLVSLPKVGWVKYRKSREIIGDLKNATISLNQGEWYISFNTEQTVPDPIHPSDIKTTIVLNNVNSV |
| | HLSSGVGGDNTYQAEEKKKLIRLNKTLTRRKKYSKNWLKTKAKIDRVRSKAARIRLDNIHKATTAICKN |
| | HAVVEVVNLMDSVSAKNDNTLSMRYEFVRQLIYKQEWLGGEIIRRESKLL |
| 390 | MTLRCLLNPWRFKQAFKFLLEPNKGQLSDFLAFAGSCRFVYNKGLALLNENYRSGKKFIGYNQLASELV |
| | EWKNEESLSWLKEAPSQCLQQSLRDLDKAFRNFFTGKSQYPKFKKKGRHDSFRTPSQRVRVDQEKKLV |
| | SLPKVGWVKYRKSREIIAELKNVTISMKQGKWYISFNTEHTVPDPIHPSDIKTKIVLNNVNSVHLSSGIGG |
| | DNTSQAEEKKKLIRLNKRLARRKKHSKNWLKTKGKIDRVKSKAARLRLDNIHKATTAICKSHAVIEVVN |
| | LMGSVSDKNDNTLSMRYEFVRQLIYKQEWLGGEVIRRESKPL |
| 391 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 392 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIAYHPKTLIQTSRPLEVTLPKFHQTEEKLQHA |
| | QRKLNVKVRSAHHRKIRLDQASNYQKQKRKVMDLYLKQKNQREDYLEQLSGKLVKQYDYLFVESFPK |
| | EEAHADFSIHDWHKLITKLRYKSQWYNKKFLLINTDGAEESNSVRKSQVVEKMGRHSVIKG |
| 393 | MKALKAYKYRLYPTSKQEEFIQKTFSCVRLVYNLMLQDRIDIYKEMRKNPQQTFKMPTPAQYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPVETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 394 | MKLSVLKAYKFRIYPTEEQKQFFIQTFGCVRFTYNQLLKAKMEELTTNAEKEKLTPAKLKKEYPFLKET |
| | DSLALANAQRNLERAFRNYFQKRAGFPKLKTKKNIWQSYTTNNQQHTIYLVDDQLKLPKLKSFVAVKR |
| | HRPINGQIKSATISARNNTEFYISILCIEEIQPLPKNQRKIALVYHPEVLVEANAQLPFISTNAIKSQQRLARA |
| | ERKLNVKAKAVKRKKMVLSHARNYQKQKGKVSQLYRAHRDQKKEYIDQVTFHLVKQYDTIFLERLID |
| | ETCRSTGNFSVSDWHQFIRKITYKAEWYGKEVRFISLSAKECQKMTQMLRVIESETNWEERQGSPRG |
| 395 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIAYHPKTLIQTSRPLEVTLPKFHQTEEKLQHA |
| | QRKLNVKVRSAHHRKTRLDQASNYQKQKRKVMDLYLKQKNQREDYLEQLSGKLVKQYDYLFVESFP |
| | KEEAHADFSIHDWHKLITKLRYKSQWYNKKFLLINTDGAEESNSVRKSQVVEEMGRHSLIKG |
| 396 | MKALKAYKYRLYPTSKQEEFIQKTFSCVRLVYNLMLQDRIDIYKEMRRNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 397 | MKALKAYKYRLYPTSKQEQFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 398 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPVETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 399 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALISNGESFEKSYCSKH |
| | LKQKLRQEERKLNKRKMIALEKGVDLSQAKNYQKQKIKVAKIREKIANQRTDILNKITTELVSSYDVICIE |
| | KAHHSNERPPKHDRSELAWSLFLAKLLYKAQWYGKELICIESEEIETELSFSESTENSEYLRSQKILERGLS |
| | KRETL |
| 400 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIVYHPKTLIQTSRPLEVTLPKFHQTEEKLQHA |
| | QRKLNVKVRSAHHRKIRLDQASNYQKQKRKVMDLYLKQKNQREDYLEQLSGKLVKQYDYLFVESFPK |
| | EEAHADFSIHDWHKLITKLRYKSQWYNKKFLLINTDGAEESNSVRKSQVVEKMGRHSVIKG |
| 401 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSSVKKESLAKWIGIAYHPKTLIQTSRPLEVTLPKFHQTEEKLQHA |
| | QRKLNVKVRSAHHRKIRLDQASNYQKQKRKVMDLYLKQKNQREDYLEQLSGKLVKQYDYLFVESFPK |
| | EEAHADFSIHDWHKLITKLRYKSQWYNKKFLLINTDGAEESNSVRKSQVVEEMGRHSVIKG |
| 402 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEISPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 403 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRMDILNKITTELVSSYDVI |
| | CIEKAHHSNERPPKHDRSELAWSLFLAKLLYKAQWYGKELICIESEEIETELSFSESTENSEYLRSQKILER |
| | GLSKRETL |
| 404 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSNYDVIC |
| | IEKAHHSNERPPKHDRSELAWSLFLAKLLYKAQWYGKELICIESEEIETELSFSESTENSEYLRSQKILERG |
| | LSKRETL |
| 405 | MIEEEIGVRGGEMSVLKGYKFRIYPDEKQKKFFIETFGCVRFTYNHLLMARHTGTARNTTLTPASLKKEY |
| | PFLKKTDSLALANAQRNLERAFRNYFSGRAGYPKLKTKKSTWQSYTTNNQQHTVYLEGEYLKVPKLKS |
| | LVPIHLHREVRGTIKSVTISAKRNREFYASILCVEEVEELPKTNDLVGISYCPENLIQISAKKELPQIDQSHL |
| | VKQLGKEQKKLQLRAKVAKKRKVRLIHAKNYQKQKERVLKLRATKLDQKRNFIDQLTINLVRDFDYLF |
| | IESKPKFKNETGEFSEADWQQFIQRIQYKGRWYGKEIRYIEVKELKNEKCKEIERLGRAQLT |
| 406 | MEQLKAYKFRIYPTEEQEIFFAKSFGCVRKVYNLMLDDRKKAYEEVKNDSSKKMTFPTPAKYKKEFPFL |
| | KEIDSLALANAQLNLDKAYKNFFRDKSVGFPRFKSKKNPVQSYTTNNQNGTIALIDSKFIKVPKLKSLIRI |
| | KLHRQPKGMIKSATISRHSSGKYYISLLCKEEISELPKTNSAIGIDLGITDFAILSDGQKIDNNKFTSKMEKK |
| | LKREQRKLSRRALLAKQKGINLFEAKNYQKQKRKVARLHEKVMNQRTDFLNKLSTEIIKNHDIICIEDLN |
| | VKGMLRNHKLAKSISDVSWSKFVTKLQYKADWYGRKIIKVDK |
| 407 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEIPPLPKQQASVAVVYDPQQLVKANQPVPITCEHAIQTKQKLT |
| | RAERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVILDTQDQQKLERLSGEMSS |
| 408 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIVLEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 409 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYMENGYLKLPKQKELIKINQHR |
| | PVEGSIRSATISARYNEEFYVALLCDVSPVKKESLAKWIGIAYHPKTLIQTSRPLEVTLPKFHQTEEKLQH |
| | AQRKLNVKVRSAHYRKIRLDQASNYQKQKRKVMDLYLKQKNQREDYLEQLSGKLVKQYDYLFVESFP |
| | KEEAHADFSIHDWHKLITKLRYKSQWYNKKFLLINTDGAEESNSVRKSQVVEKMGRHSVIKG |
| 410 | MKALKAYKYRLYPTSKQKEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 411 | MKVLKAYKYRLYPTSLQEEFIKKTFSCVRLVYNLLLQERIQLYKELKENPDLKVKMPTPAQYKKEYPCL |
| | KEVDSLALANAQVYLDRAFKKFHREKAVGFPKLKQKKNAVCSYTTNNQNGTIKIIDEKYLKVPKLKSL |
| | MKMKMHRPVIGKIKSATISLTPSNKYFVSILCEEEIPAVEKTHFAIGITLGASEFAVLSNGRRFDNDKYTK |
| | EFERRITREERKLRRRKEIAKIKGTDLSQQKNYQKQKVKVVKMREKLMNQRIDFLNKITTEIVRKYDLICI |
| | EDIHQADFYRNNKLHRGVTDVSWALFVSKLEYKASWYNKRLIKVSVCHKCSEHSDNTRISKLFFHEINE |
| | KKGRQDPETAASIQVLTQGLKEATVND |
| 412 | MKALKAYKYRLYPTSKQEEFIQKTFSCVRLVYNLMLQDRIDIYKEMRKNPQQTFKMPTPAKYKKHYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 413 | MKALKAYKYRLYPTSKQEQFIQKTFSCVRLVYNLMLQERIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 414 | VKVLKAYKFRIYPNEEQIQYFIQTFGCVRFTYNQLLYARKKALQAGDYVTRLTPAQLKKDYPFLKQTDS |
| | LALANAQRNLDRAFKNYFSKRAGYPKWKSKKSHWQSYTTNNQKHTIYFIGEELKLPKLKSLVKANLHR |
| | EILGEIKSATISAKNNQLFFVSILCLENVMSLPKTGESIGVAYCSENLVQMSSTNVFLSRKSNSYYQLKTA |
| | KKRLELRAKLAKKRKVLLSQAKNYQKQKRKVQKLYMIIDNQKNDYINQLTYFLVKNYDYIYLEKHPKF |
| | SENAKFSETDWQHLLRKIQYKVSWYNKQLAFVAPDTKESEEKCFTIEQLGRQLTTS |
| 415 | MKVLKAYKYRLYPTSLQEEFIKKTFSCVRLVHNLLLQERIQLYKQLKENPDLNVKLPTPAQYKKEHPCL |
| | REVDSLALSNAQVYLDRAFKKFHREKSVGFPKLKQKKNAVNSYTTNNQNGTVKIIDGKYLKVPKLKSLI |
| | KMKMHRPVIGKIKSATISLTPSNKYFVSILCEEEIPTVEKTHSAIGITLGVSEFAVLSNGRRIDNDKYTKEFE |
| | QRITREERKLMRRKEIAKSKGTELSQQKNYQKQKLKIVKMREKLMNQRIDFLNKITTEIVRKYDLICIEDI |
| | QQADFYRNNKLHRGVTDVSWALFVSKLEYKASWYNKRLIKVSACGKCSEHSDNKELSQIFFQDINTKK |
| | SKNDPETAASVQVLIRGLQEVVQ |
| 416 | MKVLKAYKYRLYPTLLQEEFIKKTFSCVRLVHNLLLQERIQLYKELKNNPDLKVKLPTPAQYKKEHPCL |
| | KEVDSLALSNAQVYLDRAFKKFHREKTVGFPRLKQKKNAVTSYTTNNQNGTIKIIDEKYLKVPKLKSLIK |
| | MKLHRPVIGKIKSATISLTPSNKYFVSILCEEEIPKVEKTYSAIGITLGASEFAVLSNGKRIDNDKFTKEFEQ |
| | RITREERKLTRRKEIAKSKNTELSQQKNYQKQKLKVAKMREKLMNQRIDFLNKITTEIVRQYDLICIEDIH |
| | QADFFRNSKLHRGVSDVSWALFVSKLEYKAAWYKKRLIKVSACGKCSEHSDNSLVSQIFTQDINEKKGQ |
| | HDPETAASIQVLIQGLKDTKAN |
| 417 | MKVLKAYKYRLYPTLAQEEFIKKTFSCVRFVYNLLLQDRISLYKALKENPSLTVKLPTPAHYKKEHPFLK |
| | EVDSLALANAQVYLDRAFKKFHREKSVGFPKLKQKKDSVSSYTTNNQNGTIKIIDDKYVKVPKLKSVVK |
| | VKMHRPLKGKIKSATISLTPSHKYFISILCEEEVPSVAKTYSAIGITLGTSEFAVLSNGRRIDNDKYTKAFK |
| | QRIAREERKLTRRKEIAKLKGVELSQQKNYQKQKLKVAKMREKLMNQRTDFLNKITTEIVQKYDVICIE |
| | DIQQSDIYRNSKLHCGISDVSWAMFVSKLEYKATWYNKRLIKVSMCNECSEHSDNNKRSNLFIQDIDKQ |
| | KGQCDPETAASIQVLNKGLSS |
| 418 | MKVLKAYKYRLYPNPLQEEFIRKTFSCVRLVHNLLLQDRVEIYRKLKKDSKLKIKYPTPAKYKKDYPFL |
| | KEVDSLALSNAQVHLDRAFKNFHKNKSVGFPKLRQRKDSVSSYTTNNQNGTIKILDSKYLKVPKLKTLI |
| | KMKVHRPLTGEIKSATISLSPSKKYFVSLLCEEEIPKAPKTYSAVGITLGTSEFAILSNGQRIDNDKYTQNF |
| | QIRLKREEKKLIRRKEIAQSKKMDISQQKNYQKQKLKIAKMHEKLMNQRIDFLNKITTEIVTKYDVICVE |
| | DIHKEDFFRNSKLNRGITDVSWAMFISKLEYKALWYNKKIIKVSACQDSSVIVEETESKLFTPDVNKKKA |
| | LEDPEIAASVQVLSMGLNEAIAN |
| 419 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHDRSELAWSLFLAKLLYKAQWYGKKLICIESEEIETELSFSESTENSEYLRSQKILERG |
| | LSKRETL |
| 420 | VHNLLLQERIKLYKQLKEDPNLKVKLPTPAQYKKEYPCLKEVDSLALSNAQVYLDRAFKKFHREKSIGF |
| | PKLKQKKDSVCSYTTNNQNGTVKIIDEKYLKVPKLKSLVRMKMHRPVIGKIKSATISLAPSNKYFVSILC |
| | EEEIPTIEKTYSAVGITLGASEFAILSNGRRIDNDKFTKEFEQRITREERKLTRRKEIAKAKGTDLSQQKNY |
| | QKQKLKVAKMREKLMNQRIDFLNKITTEIVRKYDLICIEDIHQADFYRNSKLHRGISDVSWALFVSKLEY |
| | KATWYNKRVIKVLACGKCSEHSENNVSQIFTQDINEQKGLQDPETAASINVLIQGLKETTGN |
| 421 | VHNLLLQERIQLYKELKKNPDLKVKLPTPAQFKKEHPCLKEVDSLALSNAQVYLDRAFKKFYREKSVGF |
| | PKLKQKKNAVSSYTTNNQNGTIKIIDEKYLKVPKLKSLIKMKMHRPVIGKIKSATISLTPSNKYFVSILCEE |
| | ELPRVEKTYSAIGITLGASEFAVLSNGRRIDNDKFTKEFEQRITREERKLTRRKEIAKSKGTELLQQKNYQ |
| | KQKLKVAKMREKLMNQRIDFLNKITTEIVKKYDLICIEDIHQADFFRNTKLHRGVSDVSWALFVSKLEY |
| | KATWYNKRLIKVSACGKCSEHSDNDLVSQIFTQDVNEEKGKHDPETAASIQVLIQGLKGTTAN |
| 422 | MQERVQLYKELKENPDLKVKLPTPAQYKKEHPCLKEVDSLALSNAQVYLDRAFKKFYREKSVGFPKLK |
| | QKKDSVSSYTTNNQNGTVKIIDEKYLKVPKLKSLLKMKMHRPVIGKIKSVTISLTKSNKYFVSILCEEEIPI |
| | IEKTHSAIGITLGASEFAVLSNGNRIDNDKYTKEFEQRITREERKLQRRKEIAKVKGTDLSQQKNYQKQKL |
| | KVAKMREKLMNQRVDFLNKITTEIIRKYDLICIEDIHQADVYRNNKLYRGVSDVSWALFVSKLEYKASW |
| | YNKRLIKVSACGKCSEHSDNTQVSQMFTQDINEQKGLHDPETAASIQVLIKGLKETTRK |
| 423 | MKENPDLKVKLPTPAQYKKEHPCLKEIDSLALSNAQVYLDRAFKKFHREKSVGFPKLKQKKNAVRSYT |
| | TNNQNGTIKIIDGRYLKVPKLKSLIKMKMHRQMVGKIKSATISLTPSQKYFVSILCEEEVPTVEKTYAAIGI |
| | TLGSSEFAVLSNGKRIDNDKYTKEFETRINREERKLMRRKEIAKSKGIELSQQKNYQKQKLKVAKMREK |
| | LMNQRIDFLNKVTTEIVRKYDLICIEEIHQADVFRNNKLHRGVSDVSWALFVSKLEYKASWYNKRLIKV |
| | SICGKSSEHSDNDMSSRLFFQDINEKRAMIDPETATSVQVLTQGLKEVVI |
| 424 | VHNLLLQERIQLYKKLKENPNLKVKMPTPAQYKKEHPCLREVDSLALANAQVYLDRAFKKFHREKSVG |
| | FPKLKQKKNAVCSYTTNNQNGTIKIIDEKYLKVPKLKSLMKMKMHRPVIGKIKSATISLTPSNKYFVSILC |
| | EEEIPAVEKTHFAIGITLGASEFAVLSNGRRFDNDKYTKEFERRITREERKLRRRKEIAKLKGTDLSQQKN |
| | YQKQKTKVAKMREKLMNQRIDFLNKITTEIVRKYDLICIEDIHQADFYRNNKLHRGVTDVSWALFVSKL |
| | EYKASWYNKRLVKVSVCQKCSEHSDNNRMSKIFFHDINEKKGRQDPETAASIHVLTQGLKEATVTD |
| 425 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEEDQIKLPKLKTLVPVKK |
| | HREIKGKIKSATISAKNNEEFYISILCLEEITPLPKQQASIAIVYDPQQLVKANQPVPITCEHAIQTKQKLTR |
| | AERKLQVKATAVKRKKILLTQARNYQKLKGKVTRLYRFHCCQKREFIDQVSYHLVKQYDTIYLEQIAED |
| | TVLKAGHYSISDWHQFVRKIQYKAQWYGKELRFVTLDTQDQQKLERLSGEMSS |
| 426 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSSERPPKHDRSELAWSLFLAKLLYKAQWYGKELICIESEEIETELSFSESTENSEYLRSQKILERG |
| | LSKRETL |
| 427 | MVYNLMLQDRIDIYKEMRKNPQQTFKMPTPAKYKKQYPVLREVDSLALANAQVYLDRAFKNFYREKG |
| | MGFPKKKKKETVHSYTTNNQHGTVKILDNRYLKVPKLKSLIKMKVHRQPLGEINSVTISMSASHNYYVS |
| | ILCEAPIETKTKQQKMVGICSSREKFALLSNGESFEKSYCSKHLKQKLRQEERKLNKRKMIALEKGVDLS |
| | QAKNYQKQKIKVAKIREKIANQRTDILNKITTELVSSYDVICIEKAHHSNERPPKHDRSELAWSLFLAKLL |
| | YKAQWYGKELICIESEEIETELSFSESTENSEYLRSQKILERGLSKRETL |
| 428 | MKALKAYKYRLYPTSKQEQFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPVETKTKQQKMVGICSSREKFALLSNGESFEKSYCSK |
| | HLKQKLRQEERKLNKRKMIALEKGFDLSQAKNYQKQKIKVAKIREKIANQRTDILNKITTELVSSYDVICI |
| | EKAHHSNERPPKHDRSELAWSLFLAKLLYKAQWYGKELICIESEEIETELSFSESTENSEYLRSQKILERGL |
| | SKRETL |
| 429 | MKVLKAYKYRLYPTSIQEEFIKKTFSCVRLVHNLLLQERIQLYKQLKENPDLKVKLPTPAQYKKEYPCLK |
| | EVDSLALSNAQVYLDRAFKKFHREKSIGFPKLKQKKDSVSSYTTNNQNGTVKIIDEKYLKVPKLKSLVK |
| | MKMHRPVIGKIKSVTISLTPSNKYFASILCEEEIPTIEKTYSAVGITLGASEFAVLSNGRRIDNDKFTKEFEQ |
| | RITREERKLTRRKEIAKAKGTDLSQQKNYQKQKLKVAKMREKLMNQRIDFLNKITTEIVRKYDLICIEDI |
| | HQADFYRNSKLHRGISDVSWALFVSKLEYKATWYNKRVIKVLACGKCSEHSENRVSQIFTQDINEQKGL |
| | QDPETAASINVLIQGLKKTTGN |
| 430 | MKVLKAYKYRLYPTALQEEFIKKTFSCVRLVHNLLLQERIQLYKELKKTPDLKVKLPTPAQFKKEHPCL |
| | REVDSLALSNAQVYLDRAFKKFYREKSVGFPKLKQKKNAVRSYTTNNQSGTIKLIDKKYLKVPKLKSLI |
| | KIKMHRPVMGKIKSATISLTPSNKYFVSILCEEEIPTVEKTYSAVGITLGASEFAVLSNGRRIDNDKFTKDF |
| | EQRITREERKLLRRKEIAKLKGNELSQQKNYQKQKLKVAKMREKLMNQRVDFLNKITTEIVRKYDLICIE |
| | DIHQADFFRNNKLHRGISDVSWALFVSKLEYKATWYNKRLVKVTSCGESSEHSDNNALSKIFTQDINEK |
| | KGQKDPETAASIQVLIKGLRDTKNG |
| 431 | MLKAFKFRMYPTEEQKQQLIRTFGCARFTYNHLLKKRQKSWQQTGVADFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIAYHPKTLIQTSRPLEVTLPKFHQTEEKLQHA |
| | QRKLNVKVRSAHHRKIRLDQASNYQKQKRKVMDLYLKQKNQREDYLEQLSGKLVKQYDYLFVESFPK |
| | EEAHADFSIHDWHKLITKLRYKSQWYNKKFLLINTDGAEESNSVRKSQVVEKMGRHSVIKG |
| 432 | MKVLKAYKYRLYPTLSQEVFIKKTFSCVRLVHNLLLQERIQLFKTLKEQPELKVKMPTPAQFKKEHPCL |
| | KEVDSLALSNAQVYLDRAFKKFHREKSVGFPKLKQKKDAVSSYTTNNQNGTIKIIDEKYLKVPKLKSLIK |
| | MKMHRPIIGKIKSATISLSPSNKYFVSILCEAEIPTVEKTYSAIGITLGTSEFAVLSNGRRIDNDKFTKEFEQ |
| | RITREERKLTRRKEIAKVKGTDITQQKNYQKQKLKVAKMREKLMNQRIDFLNKITTELVRKYDLICIEDI |
| | HQNDFFRNSKLHRGVSDVSWALFVSKLEYKVSWYNKRLIKVSACGKCSEHSDNTQLSQMFTQDINAKK |
| | GQNDSETAASIQVLVQGLKNIRT |
| 433 | VKVLKACKYRLYPTSSQIEFFEKTFSSVYLVHNLLLQDRINLYREAKKNPQLKQSLPTPAKYKREHPLLK |
| | EVDSLALANAQVHLERSLKRFYSGKDVGFPKMKSRKNPVMSYTTNNQNGTIKFVGLNCLKIPKLKSLIK |
| | VKMHREVKGKIKSATISKSSTGKYFVSILCEETIACQKKTNKAVGISLGCSELAVLSNGRRIDNDCLTEEIE |
| | RKIRREEKKLARKKKLASQKGLDLLEQKNYQKQKMKVAKLRERLLNQRNDFLNKVTTDLIKEYDLICIE |
| | EAHKKEFHRNCKLTKRVSDVSWSLFVSKLEYKAMWHDKRLIKIKKNDQIEATKIPTKMILDIDQELSESD |
| | TETASSIQLLLQGLNQ |
| 434 | MRVLKAYKYRLYPTSAQEEFIKKTFSCVRLVYNLLLQDRIALYKALKENPDLTVKLPTPAQYKKEHPCL |
| | KEVDSLALANAQVYLDRAFKKFHREKGVGFPKLKQKKDSVSSYTTNNQNGTIKIIDDKCIKVPKLKTPM |
| | KVKMHRPIKGKIKSATISLTPSHKYFISILCEEEVPEVEKTYSAIGITLGTSEFAVLSNGRRIDNDKYTREFE |
| | QRLAREERKLVRRKEIAKVKGIELSQQKNYQKQKLKVAKMREKLMNQRTDFLNKITTEIVRKYDVICIE |
| | DIHQTEVYRNRKLHRGISDVSWALFVSKLEYKASWYNKRLIKVSACNECSEHSENKKQSKIFLEDIDKQ |
| | KGASDPETAASIHVLNKGLSY |
| 435 | MRVLKAYKYRLYPTSAQEEFIKKTFSCVRLVYNLLLQDRIALYKALKENPDLTVKLPTPAQYKKEHPCL |
| | KEVDSLALANAQVYLDRAFKKFHREKGVGFPKMKQKKDSVSSYTTNNQNGTIKIIDDKWIKVPKLKTP |
| | MKVKMHRPIKGKIKSATISLTPSHKFFISILCEEEVLAVEKTHSAIGITLGTSEFAVLSNGRRIDNDKYTKEF |
| | EQRLIREERKLVRRKEIAKLKGTELSQQKNYQKQKLKVAKMREKLMNQRTDFLNKITTEIVHKYDVICIE |
| | DIHQSDVYRNSKLHRGISDVSWALFVSKLEYKASWYNKRLIKVSACDKCSEHSENKKRSKIFIEDIDKQK |
| | EVSDLETAASIHVLNKGLSY |
| 436 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEEDQIKLPKLKTLVPVKK |
| | HREIKGKIKSATISAKNNEEFYISILCLEEITPLPKQQASIAVVYDPQQLVKANQPVPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVILDTQDQQKLERLSGEMRS |
| 437 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIAYHPKTLIQTSRPLEVTLPKFHQTEEKLQHA |
| | QRKLNVKVRSAHHRKIRLDQASNYQKQKRKVMDLYLKQKNQREDYLEQLSGKLVKQYDYLFVESFPK |
| | EEAHADFSIHDWHKLITKLRYKSQWYNKKFLLINTDEAEESNSVRKSQVVEKMGRHSVIKG |
| 438 | VKVLKAYKFRIYPNEEQIQYFIQTFGCVRFTYNQLLYARKKALQAGDYVTRLTPAQLKKDYPFLKQTDS |
| | LALANAQRNLDRAFKNYFSKRAGYPKWKSKKSHWQSYTTNNQKHTIYFIGEELKLPKLKSLVKANLHR |
| | EILGEIKSATISAKNNQLFFVSILCLENVMSLPKTGESIGVAYCSENLVQMSSTNVFLSRKSNSYYQLKTA |
| | KKRLELRAKLAKKRKVLLSQAKNYQKQKRKVQKLYMIIDNQRNDYINQLTYFLVKNYDYIYLEKHPKF |
| | SENAKFSETDWQHLLRKIQYKVSWYNKQLAFVAPDTKESEEKCFTIEQLGRQLTTS |
| 439 | MKALKAYKYRLYPTSKQEQFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIVLEKGVDLSQAKNYQKQKIKVAKIREKIANQRTDILNKITTELVSSYDVIC |
| | IEKAHHSNERPPKHDRSELAWSLFLAKLLYKAQWYGKELICIESEEIETELSFSESTENSEYLRSQKILERG |
| | LSKRETL |
| 440 | VERLKAYKFRIYPTEEQEIFFAKTFGCVRKVYNLMLNDRKKAYEEVKNDPSKKMAFPTPAKYKKEFPFL |
| | KEVDSLALANAQLHLDKAYKNFFRDKSVGFPRFKSKKNPVQSYTTNNQKGTIALIGSKFIKLPKLKSLVR |
| | IKLHRQPKGMIKSATISRHSSGKYYISLLCKEEISELPKTNSAIGIDLGITDFAILSDGQKIDNHKFTSKMEK |
| | KLKREQRKLSRRALLAKQKGINLFEAKNYQKQKRKVARLHEKVMNQRTDFLNKLSTEIIKNHDIICIEDL |
| | NVKGMLRNHKLARSISDVSWSSFVAKLQYKADWYGREIIKVNQWFPSSQICSECGHKDGKKPLDI |
| 441 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFKDDQIKLPKLKTLVPVKK |
| | HRAIKGKIKSATISAKNNEEFYISILCLEEIPQLPKQQASVAVVYDPQQLVKANQPIPITCEHAIQTKQKLT |
| | RAERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTHDQQKLERLSGEMSS |
| 442 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEIPPLPKQQASVAIVYDPQQLVKANQPVPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 443 | VKVLKGYKFRIYPNEEQIQFFIQTFGCVRFTYNCLLCARKESLQKRSYETKLSPAHLKKDYPFLKQADSL |
| | ALANAQRNLDRAFKNYFSKRMGYPKFKTKNNTWQSYTTNNQKNTIYLVGKQLKLPKLKSLVSVNLHR |
| | EVFGEIKSATISAKNNQLFFVSLLCLEEVFPLPKTGKAIGIAYCPKHLVQLTSDRSLPVYECKGVQHRLKR |
| | ANKKLELRAKVAKKRAVVVKQAKNYQKQKHKVQKLTVKKNNQKRNYIDQLTHLLVHEYDSIYLEENP |
| | HFIDNTHFLEADWHHFLRTIRYKAHWYNKKLIFVEDVKKFDEFVMN |
| 444 | MLKAFKFRMYPTEEQKQQLIRTFGCARFTYNHLLKKRQKSWQQSGVADFSLTPATLKKEYPFLKEVDSL |
| | ALANAQLNLDRAFRNYFKGRASFPKLKTKKSMWQSYTTNNQTRTIYLENGYLKLPKQKELIKINQHRPV |
| | EGSIRSATISARYNEEFYVALLCDVSPVKKESLAKWIGIAYHPKTLIQTSRPLEVTLPKFHQTEEKLQHAQ |
| | RKLNVKVRSAHHRKIRLDQASNYQKQKRKVMDLYLKQKNQREDYLEQLSGKLVKQYDYLFVESFPKE |
| | EAHADFSIHDWHKLITKLRYKSQWYNKKFLLINTDGAEESNSVRKSQVVEKMGRHSVIKR |
| 445 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEEDQIKLPKLKTLVPVKK |
| | HREIKGKIKSATISAKNNEEFYISILCLEEITPLPKQQASIAVVYDPQQLVKANQPVPITCEHAIQTKKKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 446 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEEDQIKLPKLKTLVPVKK |
| | HREIKGKIKSATISAKNNEEFYISILCLEEITPLPKQQASIAVVYDPQQLVKANQPVPITCEHAIQTKQKLIR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVILDTQDQQKLERLSGEMSS |
| 447 | MLKAFKFRMYPTEEQKQQLIRTFGCARFTYNHLLKKRQKSWQQTGVANFSLTPATLKKEYPFLKEVDS |
| | LALANAQLNLDRAFRNYFKGRASFPKLKTKKSMWQSYTTNNQTRTIYLENGYLKLPKQKELIKINQHRP |
| | VEGSIRSATISARYNEEFYVALLCDVSPVKKESLAKWIGIAYHPKTLIQTSRPLEVTLPKFHQTEEKLQHA |
| | QRKLNVKVRSAHHRKIRLDQAGNYQKQKRKVMDLYLKQKNQREDYLEQLSGKLVKQYDYLFVESFPK |
| | EEAHADFSIHDWHKLITKLRYKSQWYNKKFLLINTDGAEESNSVRKSQVVEKMGRHSVIKG |
| 448 | MKLGVLKAYKFRIYPNGQQRQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEISPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 449 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HRAIKGKIKSATISAKNNEEFYISILCLEEIPPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSNEMSS |
| 450 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQQHTIYFEDDQIKLPKLKTLVPVKK |
| | HREIKGKIKSATISAKNNEEFYISILCLEEIPPLPKQQASVAVVYDPQQLVKANQPVPITCEHAIQTKQKLT |
| | RAERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVILDTQDQQKLERLSGEMSS |
| 451 | MKALKAYKYRLYPTSKQEEFIQKTFSCVRLVYNLMLQDRIDIYKEMRKNPQQTFKMPTPAKYKKQYPV |
| | LREVDSLALANAQVYLDRAFKNFYREKGMGFPKKKKKETVHSYTTNNQHGTVKILDNRYLKVPKLKSL |
| | IKMKVHRQPLGEIKSVTISMSASHNYYVSILCEAPIETKTKQQKMVGICSSREKFALLSNGESFEKSYCSK |
| | HLKQKLRQEERKLNKRKMIALEKGVDLSQAKNYQKQKIKVAKIREKIANQRTDILNKITTELVSSYDVIC |
| | IEKAHHSNERPPKHYRSELAWSLFLAKLLYKAQWYGKELICIESEEIETELSFSESTENSEYLKSQKILERG |
| | LSKRETL |
| 452 | MVKILKACKYRLYPTSSQIEFFEKTFSSVYLVHNLLLQDRINLYKEAKKNPNSKKSPPTPAKYKREYPLL |
| | KEVDSLALANAQVHLERALKRYYSGKDVGFPKMKSKKNPVNSYTTNNQNGTIKFVGLNCLKIPKLKSLI |
| | KVKVHREPKGKIKSATISRSSTGKYFVSILCEETICCQKKTNRAVGISLGCSELAVLSNGRRIDNDHLTEEI |
| | ERKIQREEKKLARKKYLASAKGLDLLEQKNYQKQKMKVARLRERLLNQRNDFLNKVTTHLVKEYDLIC |
| | IEDAHKKEFNRNCKLTKRVSDVSWSLFVSKLEYKAMWHDKQLIKLKKNDDKIVTTASTEMTLDIDPELS |
| | KNDPETAASIQLLLQGLNQ |
| 453 | MKVLKAYKYRLYPTLLQEEFIKKTFSCVRLVHNLLLQERTQLYKELKNNPDLKVKLPTPAQYKKEHPCL |
| | KEVDSLALSNAQVYLDRAFKKFHREKSVGFPKLKQKKNAVTSYTTNNQNGTIKIIDEKYLKVPKLKSLIK |
| | MKLHRPVIGKIKSATISLTPSNKYFVSILCEEEIPKVEKTYSAIGITLGASEFAVLSNGKRVDNDKFTKEFE |
| | QRITREERKLTRRKEIAKSKNTELSQQKNYQKQKLKVAKMREKLMNQRIDFLNKITTEIVRKYDLICIEDI |
| | HQADFFRNSKLHRGVSDVSWALFVSKLEYKAAWYKKRLIKVSACGKCSEHSDNSLVSQIFTQDINEKKG |
| | QHDPETAASIQVLIQGLRDTKAN |
| 454 | MKVLKAYKYRLYPTLLQEEFIKKTFSCVRLVHNLLLQERIQLYKELKKNPDLKVKLPTPAHYKKEHPCL |
| | REVDSLALSNAQVYLDRAFKKFHREKSVGFPKLKQKKNAVRSYTTNNQNGTIKIIDDKYLKVPKLKSLI |
| | KMRMHRPVIGKIKSATISLTPSNKYFVSILCEEEIPRIDKTYSAIGITLGASEFVVLSNGRRIDNDKFTKEFE |
| | QRITREERKLKRRKEIAKLKGTELSRQKNYQKQKLKVAKMREKLTNQRIDFLNKITTEIVKKYDLICIEDI |
| | QQADFFRNSKLQRGFSDVSWALFVSKLEYKATWYNKRMIKVSACGKCSEHSDNNPVSQIFMQDIDEKT |
| | GRHDPETAASIQVLMQGLKEIMTY |
| 455 | MKVLKAYKYRLYPTSLQEEFIKKTFSCVRLVHNLLLQERIQLYKNLKENPNLKVKLPTPAQYKKEHPCL |
| | KEVDSLALSNAQVYLDRAFKNFHREKSVGFPKLKQKKNSVTSYTTNNQNGTVKIIDEKYLKVPKLKSLI |
| | KMKMHRPIMGKIKSATISLTPSKKYFVSILCEEDIPKVEKTYSAIGITLGASEFAVLSNGRRIDNDKYTKEF |
| | EQRITREERKLTRRKEIAKVKGIELSQQKNYQKQKLKVAKMREKLMNQRIDFLNKITTEIVRKYDLICIED |
| | IHRADFFRNNKLHRGISDVSWALFVSKLEYKASWYNKRLIKVSACGKCSEHSDNKSVSQIFIQDIDEKKG |
| | IQDPETAASIQVLVQGLKESVAN |
| 456 | MKVLKAYKYRLYPTSLQEEFIKKTFSCVRLVHNLLLQERIQLYKNLKENPGLKVKLPTPAQYKKEHPCL |
| | KEVDSLALSNAQVYLDRAFKNFHREKSVGFPKLKQKKNSVTSYTTNNQNGTIKIIDEKYLKVPKLKSLIK |
| | MKMHRPVIGKIKSATISLTPSKKYFVSILCEEDIPIVEKTYSAIGITLGASEFAVLSNGRRIDNDKYTKEFEQ |
| | RITREERKLTRRKEIAKVKGIELSQQKNYQKQKLKVAKMREKLMNQRIDFLNKITTEIVRKYDLICIEDIH |
| | RADFFRNNKLHRGISDVSWALFVSKLEYKASWYNKRLIKVSACGKCSEHSDNKRISQIFIQDIDEKKGIQ |
| | DPETAASIQVLVQGLKESVAN |
| 457 | MKVLKAYKYRLYPTSIQEEFIKKTFSCVRLVHNLLLQERIQLYKQLKENPDLKVKLPTPAQYKKEYPCLK |
| | EVDSLALSNAQVYLDRAFKKFHREKSIGFPKLKQKKDSVSSYTTNNQNGTVKIIDEKYLKVPKLKSLVK |
| | MKMHRPVIGKIKSVTISLTPSNKYFASILCEEEIPTIEKTYSAVGITLGASEFAVLSNGRRIDNDKFTKEFEQ |
| | RITREERKLTRRKEIAKAKGTDLSQQKNYQKQKLKVAKMREKLMNQRIDFLNKITTEIVRKYDLICIEDI |
| | HQADFYRNSKLHRGISDVSWALFVSKLEYKATWYNKRVIKVLACKKCSEHSENSVSQIFTQDINEQKGL |
| | QDPETAASINVLIQGLKETTGN |
| 458 | MKACKYRLYPTSSQIEFFEKTFSSVHLVHNLLLQDRIALYKEAKKNPQRKNSLPTPAKYKREYPLLKEVD |
| | SLALANAQVHLERALKRFYSGKDVGFPKMKSKKNPVTSYTTNNQKGTIKIVGLNCLKIPKLKTLIKLKV |
| | HREPKGKIKSATISRSSTGKYFVSILCEETIQCRKKTNRAVGITLGCSELAILSNGQRIDNDQLTKEIEGRIQ |
| | REEKKLARKKQLASEKGLDLLEQKNYQKQKMKVARLRERLLNQRHDFLNKVTTNLVNEYDLICIEDAH |
| | KKEFNRNCKLNKRVSDVSWSLFVSKLEYKAMWHDKQLIKLKRSCTEEPCAKPLTELLLDIDSENGKHD |
| | KEIASSIQLLFQGLNQ |
| 459 | MRVLKAYKYRLYPTSAQEEFIKKTFSCVRLVYNLLLQDRIALYKALKENPDLTVKLPTPAQYKKEHPCL |
| | KEVDSLALANAQVYLDRAFKKFHREKGVGFPKLKQKKDSVSSYTTNNQNGTIKIIDDKCIKVPKLKTPM |
| | KVKMHRPIKGKIKSATISLTPSHKYFISILCEEEVPEVEKTYSAIGITLGTSEFAVLSNGRRIDNDKYTREFE |
| | QRLAREERKLVRRKEIAKVKGTELSQQKNYQKQKLKVAKMREKLMNQRTDFLNKITTEIVRKYDVICIE |
| | DIHQTEVYRNRKLHRGISDVSWALFVSKLEYKASWYNKRLIKVSACNECSEHSENKKQSKIFLEDIDKQ |
| | KGASDPETAASIHVLNKGLSY |
| 460 | MVKVLKAYKYRLYPTPSQIEFFEKNFYSVSLVHNLLLQDRIMQYRASKKNPDLHLKPPTPAKYKKEYPF |
| | LKEADSLALANAQVYLERGLKYYYTGKNVGFPKLKSRKNPVTSYTTNNQGGTIKIIGLNYLKIPKLKTFV |
| | KVKAHREIKGKIKSATISKTPSGKYFVSLLCEEKIHCKEKTNRAVGISLGQTEFAVLSNGQKIDNDQLTDE |
| | IEQRIRREEKKLARKKHLAGRKGLDLLNQKNYQKQKMKVAKLREKLLNQRHDFLNKVTTELIDTYDVI |
| | CVEDAHKEDFCRNYKLNKRVSDVSWALFVSKLEYKATWHDKQLIKLKKCDEKMAVSLKNGLIGDVDL |
| | ELAKEDSETDASIQLLLQGLKNK |
| 461 | MKLGVLKAYKFRIYPNGQQKQFFIETFGCVRFTYNQLLEAKMEELANNEAKQGLTPAKLKKEYPFLKET |
| | DSLALANAQRNLDRAFRNYFQKRAGFPKMKTKKSIWQSYTTNNQHHTIYFEDDQIKLPKLKTVVPVKK |
| | HRAIKGKIKSATISAKNNEEFYISILCLEEIPPLPKQQASVAVVYDPQQLVKANQPIPITCEHAIQTKQKLTR |
| | AERKLQVKATAVKRKKILLTQARNYQKLKGKVARLYRFHCCQKREFIDQVSYHLVKQYDTIYLEQIAE |
| | DTVLKAGHYSISDWHQFVRKIQYKAQWYGKELRFVTLDTQDQQKLERLSGEMSS |
| 462 | MEQLKAYKFRIYPTEEQEIFFAKSFGCVRKVYNLMLDDRKKAYEEVKNDSSKKMTFPTPAKYKKEFPFL |
| | KEIDSLALANAQLNLDKAYKNFFRDKSVGFPRFKSKKNPVQSYTTNNQNGTVALIDSKFIKVPKLKSLVR |
| | IKLHRQPKGIIKSATISRHSSGKYYISLLCKEEVRELPKSNSAVGIDLGIIDFAILSDGQKIDNNKFTSKMEK |
| | KLKREQRKLSRRALLAKQKGINLFEARNYQTQKRKVARLHEKVMNQRTDFLNKLSTEIIKNHDIICIEDL |
| | NTKGMLRNHKLAKSISDVSWSSFVSKLQYKADWYGRKGSVAKF |
| 463 | VLKAYKFRIYPTNEQKEFLIQTFGCVRFTYNTLLKHHQQSGGGKSKKLTPASLKKEFLFLKVTDSLALAN |
| | AQQNLKRAFQNYYQGRSGYPKLKLKKSVWQSYTTNNQKQTIWLKDDLLKVPKLKQPIAVHCHRPVTG |
| | QIKSATIMAKNGQQFFVSLLCEEQITPLPKTNVTTTLHFSPDQLVSGSDLVFFRTLCQKNVENKLTKAKR |
| | KLEIKAKSAQQRGVKLSAAQNYQKQKVKVQQLYHHKQQQKKAWMDELSLHLIKKYDFLYIKVPHNIQ |
| | EGVFTLTDWQHFLVKLQYKATWYDKKVIFAAAEKVI |
| 464 | VWDISVLKAYKFRIYPTNEQKEFLIQTFGCVRFTYNTLLKHHQQSGGGKSKKLTPASLKKEFLFLKVTDS |
| | LALANAQQNLKRAFQNYYQGRSGYPKLKLKKSVWQSYTTNNQKQTIWLKDDLLKVPKLKQPIAVHCH |
| | RPVTGQIKSATIMAKNGQQFFVSLLCEEQITPLPKTNVTTTLHFSPDQLVSGSDLVFFRTLCQKNVENKLT |
| | KAKRKLEIKAKSAQQRGVKLSAAQNYQKQKVKVQQLYHHKQQQKKAWMDELSLHLIKKYDFLYIKVP |
| | HNIQEGVFTLTDWQHFLVKLQYKATWYDKKVIFAAAEKVI |
| 465 | IKILKAYKFRIYPDEAQQEFFIKTFGCVRFTYNTLLKLRQQNPSDESTLPEKMTGVWEKKTTATPAKLKR |
| | DYPFLKETDSLALANAQRNLTKAFQNYYRGRASYPKLKSKKNAWQSYTTNNQGHTIYLTNEGLKLPKL |
| | KSKVPIHQHRQVCGKIRSATISAKNRQEFYVSLLCEEEITALPKTGFDITITYDPIKLIGTSKVLSDRPNFCQ |
| | QRLLVQLKNAQRKLYCRGKSAQRRNVKLEQAKNYQKQKLRLQKLYIHQIKQKEDFMEQLSIALLRQFD |
| | LVTVTMPKAFESLSANHSAAIHQDCSANYKNTAVNFTIRDWNRFVLKLKYKANWYGKKLIFTDQEKVI |
| 466 | MSSCRTLNNKVDSMKNLKGYRFRIYPNEAQKRFFIETFGCVRFIYNYFLKLDTAERTSEEVITPASLKRD |
| | YPFLKKTDSLALANAKRNLDRAFQNYYQQRSGYPKLKNKSSAWQSYTTNNQNGTVRIEDGYLKLPKLK |
| | EKIQICEHRKITGKIKSVTISAKNNEEFYASILCVETIDKFEKTGKKIRLSFDEHQLVKQAKYRAEVIEPIQQ |
| | TKGRLEFLQRKLKVKARVARKQNRVLADCKNYQKQKKQYDKLLTHLNNQIKDYLNHLSIFYIKEYDVI |
| | EIVEPEDRSCAKDDLFTSNEWHQLTRLLKYKAQWYGKEIQIINCQNI |
| 467 | MLKAFKFRIYPTASQKEWFIQNFGCVRFTYNHLLKARQESYARTGAIDYSMTPATLKKKYAFLKSADSL |
| | ALANAQLNLDRAFRNYFKGRASFPKLKNKKSMWQSYTTNNQKGTIYLEDKYLKLPKQKELIQVRLHRP |
| | VEGVIRSATISARYNESFYVSLLCEVQIAGVPTTNRWLGVAYDPKKLVETSSPVEVQMPLFRQTRDKMK |
| | VAKRKLVIKSKAAQKRKARLENSRNYQKQKRKVMDLYQKQKLQKEDYLERVSGNLIRNYDYLFVEAV |
| | PSELSSADFQLQDWYKLITKLRYKAQWYNKTLLFINVNEQLNEPPEKKSMELEKIGKQVIFE |
| 468 | IQTFGCVRFTYNMLLTLRQQESGKTVEERTSARLQKQKMTPAKLKKDYPFLKATDSLALANAQRNLEK |
| | AFQNYYRGRASYPKLKSKKSAWQSYTTNNQGHTIYLAEDGLKLPKLKSKVLVHQHRSVAGKIRSATISA |
| | KNRQEFYVSLLCEEDIPALPKTGSEIEIAYDPTGLVVTNKPIVGIPTFCQTQVLEKLKKAQRRLSCRAKSA |
| | QRRNAKLEQAKNYQKQKSQVQQLYIHKLKQKEDFTEQLSIALLRQFDCIIITKPPELRENKESKAAKTVK |
| | KSKHTTVFPSFEDNFTLSDWNRLLLKLKYKAEWYEKELVFICPTNGK |
| 469 | MGKNQRKVLKAYKFRIYPTKAQQKFLIQTFGCVRFTYNTLLKQRQFNTIEASKKLTPAALKKEFPFLKLT |
| | DSLALANAQRNLARAFQNYYQGRSGHPKMKIKKSTWQSYTTNNQQQTIWLKDNLLKVPKLKQPIAVV |
| | CHRKVVGKIKSATITAKNLQQFYVSLLCEEEVCHLPKTKTEIELRFAPNQLVVGNQLKFCRQLCVNDLET |
| | KLKKAKRKLEIKAKSAQQRKVRLAEAKNYQKQKLKVQKLYHHKQQQKKAWIDELTMHLIKNYDFLY |
| | VEVPKNGIEGSFTLADWQSFLVKLQYKANWYGKKVIFLTAAKTVRKIS |
| 470 | MKKEDLVKVLKGYKFRIYPNEKQIQYFIQTFGCVRFTYNHLLHARQKALQAGDYQTQVSPASLKRDYPF |
| | LKKTDSLALANAQRNLDRAFKNYFSKRAGYPKLKTKKNNWQSYTTNNQKHTIYFVGNQLKLPKLKSL |
| | VTVNLHRKVAGEIKSATVSAQNNQMFFVSLLCLEEINPLPKTGTTIGVAYCPENLVQMSAVNRLPVYKQ |
| | ETLQYQLDKAIKRLEVRAKAAKRRKVLLEQAKNYQKQKSKVQKLYMAKNDQKKNYIDQLTCRLVHD |
| | YDCICLEKQPEFTENTKFSETDWQHFLRKIQYKARWYDKQLVFVDSIEKENETKCFTIEQVGKKLINQ |
| 471 | MLKAFKFRIYPTESQKQWLIQTFGCVRFTYNHLLKARQAYYLETKEIDYTLTPASLKKQYPFLKEVDSLA |
| | LANAQLNLDRAFRNYFKGRASFPKLKNKKSIWQSYTTNNQKGTIYLEETSIKLPKLKEKIRIHAHRPIEGT |
| | IRSATISSRYNEIFYVSLLCEVPQKTMEASNKWIGIAYDPDRLVEMSTPLDIAIPKFKQVDQQLQRAKRKL |
| | VIKGRAAQHRRAHVERVRNYQKQKRKIKDLYLKQKFQREDYFEQISGTVIRHYDYLFVESIPADCREGD |
| | FSIQDWHKLLAKLQYKAQWYSKKLVLIDMKEQTNPSTTKKSLELVEIGKQVLFE |
| 472 | MFLRKAATTEGIISEGRQVPIKTLKAYRFALYPDEAQKHFFIQTFGCVRFTYNMLLTLRQQESGKTVEER |
| | TSARLQKQKMTPAKLKKDYPFLKATDSLALANAQRNLEKAFQNYYRGRASYPKLKSKKSAWQSYTTN |
| | NQGHTIYLAEDGLKLPKLKSKVLVHQHRSVAGKIRSATISAKNRQEFYVSLLCEEDIPALPKTGSEIEIAY |
| | DPTGLVVTNKPIVGIPTFCQTQVLEKLKKAQRRLSCRAKSAQRRNAKLEQAKNYQKQKSQVQQLYIHKL |
| | KQKEDFTEQLSIALLRQFDCIIITKPPELRENKESKAAKTVKKSKHTTVFPSFEDNFTLSDWNRLLLKLKY |
| | KAEWYEKELVFICPTNGKENWHRNSSALSL |
| 473 | MARKSRAAEGQVIQYTTLKVRLYPTPAQAELFEKTFGCCRYIWNQMLSDQQMFYAETGAHFIPTPAKY |
| | KKGAPFLTEVDNQALIQEHNKLSQAFRVFFKRPEAFGHPNFKKKKTDRDSFTACNHVFESGPTIYTTRDG |
| | IRMTKAGVVKARFSRRAQAWWRLKRITVEKTKTQKYYCYILYEHSGKQPEPVIPTPETTVGLKYSMRHF |
| | YVADDGTTADPPRWLKQSQEKLVRVQQKLARMEPGSRNYEEAVQKYRLLHERIANQRRDFLHKESSRI |
| | ANGWDAVCMRDDALAEMSKGPLRKDAASSGFRMLRELLQYKLERQGKRLILLDRYAPTTRVCSVCGQ |
| | LQDSVDYGARTWTCPKCGTVHDREVNAAKNIKLEGLAQFLPTASPA |
| 474 | MKQQRAVKVELYPTDEQRILIHKTFGCVRAVWNDMLGDEQEFYAAADKHFIPTTAKYKKKRPYLSEVD |
| | SLALCNAQLALKKAFKRFFENPGHFGHPKFKTKKKAKKSYTTNCQYHVSGPTVYTAKDAIRLPKLGLV |
| | KAKLYRNTPDNWVLKSATISETKSGRIFCALLYEFDVPAPKEVLPTLENSIGLDYSSPLFYVDHENRSPDK |
| | PQWFRESEAKLAHEQRLLSHMKYGSKNYIRQLHKIEVLQEHIANQRKDFAHKESRRIANAYGAVCVEDL |
| | DLQAMAQSLNLGKATNDNGFGMFREFLKYKLEEQGKHLIKVDKWYPSSKTCHYCGGYYKDLQLGEEE |
| | WVCPHCGKHILRNQNAGINIRREGIRQFYAERAVEPVTFFESHAAAS |
| 475 | MKQQRAVKVELYPTDEQRILIHKTFGCVRAVWNDMLGDEQEFYAAADKHFIPTTAKYKKKRPYLSEVD |
| | SLALCNAQLALKKAFKRFFENPGHFGHPKFKTKKKAKKSYTTNCQYHVSGPTVYTAKDAIRLPKLGLV |
| | KAKLYRNTPDNWVLKSATISETKSGRIFCALLYEFDVPAPKEVLPTLENSIGLDYSSPLFYVDHENRSPDK |
| | PQWFRESEAKLAHEQRLLSHMKYGSKNYIRQLHKIEVLQEHIANQRKDFAHKESRRIANAYGAVCVEDL |
| | DLQAMAQSLNLGKATNDNGFGMFREFLKYKLEEQGKHLIKVDKWYPSSKTCHYCGGYYKDLQLGEEE |
| | WVCPHCGKHILRNQNAGINIRREGIRQFYAERAVEPVTFFESHVAAS |
| 476 | MCIQYNTIKVRLYPSVNQKELFQKTFGCCRYIWNQMLSDHERFYLETDVHFIPTPAKYKKSAPFLSKVD |
| | NQALIQEHNKLSQAFRNFFRNPGAFGYPRFKRKKDDRDTFTACNQFFGRSATIYITQNAVRMTKVGLVR |
| | AVFPRRPRSGWRLTRITVERTRTDKYYGYLLYACPVRPPQPVTPTEETTVGLNYSVSRFYVADDGTAAD |
| | PPRWLRQSQDKLCQIQRQLCRMQKGSKNYQEMVQKYRLLHEHIANQRRDFLHKESRRIANEWDAVCV |
| | RSDSLTALAAKTGGGCILDTGFGMFREMLRYKLERQGKSLLLVDRFRPTTKVCSVCGYVNEDLPAEALR |
| | WRCPVCGTEHRRERNAAANVKAIGLGRYRTETAAGGIG |
| 477 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIRAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSARPFPGVLCSALTICRPGHR |
| 478 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKESGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 479 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAAKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |
| 480 | MSSREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |
| 481 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPALERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIRAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSARPFPGVLCSALTICRPGHR |
| 482 | MSSREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |
| 483 | MSTETRKSRYTVLKVPAYPTPEQAQLMEKTFGCCRYLWNQMLSDVQEFYAATDIHYIPTPARYKKQAP |
| | FLKEVDSQALCAVHQSLRKAYLDFFRNPKVFQYPKPKTKKARKDSFTVYCRPYHTGPSLRLTNAGLQM |
| | PKLGLLQVRLYRKPLHWWSLRSVTMTKTKTGKYFCSITFGYEAELPEPVIPTPARTVGLNYSMARFYVD |
| | SNGNSPELPPQMAAAREKLARMQRKLSRMQQGSKNYEAQLHKIRLQYEHIANQRRDFAHQQSRRIANA |
| | WDAVCVRDDDLNVMAQRLKGGNVPDSGFGMFRAFLRYKLEAQGKAYIDVDPYAPAAKTCHACGHVN |
| | ENLPARARSWVCPHCGEELLREENTAQNIRDFGLMAVTRQPGVA |
| 484 | MKQQRAVKVELYPTDEQCVLIHKTFGCVRAVWNDMLGDEQEFYAATDKHFIPTPAKYKKKRPYLREV |
| | DSLALCNAQQSLKKAFKNFFENPKHFGRPCFKTKKKAKKSYTTNCQYLSSGPTVFTTKDAVRLPKLGLV |
| | KAKLYRQIPDDWVLKSATISETKSGRIFCALLYEFDVPTPAEVLPTLEGSIGLDYSSPLFYVDHENRSPDK |
| | PQWFRASEAKLAHEQRLLSHMKYGSKNYIRQLHKVQVLQEHIANQRKDFAHKESRRIANACEAVCVED |
| | LDMRAMAQSLNLGKSTNDNGFGMFREFLKYKLEEQGKHLIKVDKWYPSSKTCHYCGGYYKDLQLGEE |
| | EWTCPHCGRRILRNQNAGINIRREGIRQFYAERAAADPAAQ |
| 485 | MSTETRKSRYTVLKVPAYPTPEQAQLMEKTFGCCRYLWNQMLSDVQEFYAATDIHYIPTPARYKKQAP |
| | FLKEVDSQALCAVHQSLRKAYLDFFRNPKVFQYPKPKTKKARKDSFTVYCRPYHTGPSLRLTDTGLQMP |
| | KLGLLQVRLYRKPLHWWSLRSVTITRTKTGKYFCSITFGYEAEPPEPVAPTPARTVGLNYSMARFYVDS |
| | NGHSPELPPRMAAAREKLARMQRKLSRMQQGSKNYEAQLHKIRLQYEHIANQRRDFAHQQSRRIANA |
| | WDAVCVRGDDLNVMAQRLKGGNVPDSGFGMFRAFLRYKLEAQGKAYIDVDPYAPAAKTCHACGHVN |
| | ENLPARARSWVCPHCGEELLREENTARNIRDFGLMAVTRQPGVA |
| 486 | MSSREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQSPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 487 | MKMNDNRRPSAPKRTTQYNTIKIRLYPNQEQEELFQRTFGCCRYIWNRMLADHERFYYETDAHFIPTPA |
| | KYKTEAPFLKEVDHQALTQEYNKLSQAFRNFFRNPASFGYPKFKRKKDDRDSFSACNQVMGNSATIYIT |
| | QDAVRMTKAGLVRAKFPRRPRSGWKLTRITVERTKTGKYYGYLLFACPVHAPEPVKPTADTTIGLKYSL |
| | THFYVRDDGITADPPRWLRQSQDKVSSIQEKLNRMQPGSRNYREMVQKYRLLHEHIANQRRDFLHKES |
| | RRIANDWDAVCIRDDSLKAISEELGGSDIHDTGFGMFREMLRYKLDRQGKQLLEVGRFDPTTKVCSVCG |
| | AINETLSPKARHWVCPVCGAEHKRGKNAAVNIKAHGLACYQNKQVAEAVS |
| 488 | MKQQRAVKVELYPTDEQRILIHKTFGCVRAVWNDMLGDEQEFYAAADKHFIPTPAKYKKKRPYLSEVD |
| | SLALCNAQLALKKAFKRFFKNPGHFGHPKFKTKKKAKKSYTTNCQYHVSGPTVYTAKDAIRLPKLGLV |
| | KAKLYRNTHDNWVLKSATISETKSGRIFCALLYEFDVPVPKEVLPTLENSIGLDYSSPLFYVDHENRSPD |
| | KPRWFRESEAKLAHEQRMLSHMKYGSKNYIRQLRKIEVLQERIANQRKDFAHKESRRIANAYGAVCVE |
| | DLDLQAMAQSLNLGKSTNDNGFGMFREFLKYKLEEQGKHLIKVDKWYPSSKTCHYCGGYYKDLQLGE |
| | EEWICPHCGKHILRNQNAGINIRREGIRQFYAERAVEPVTFFESHVAAS |
| 489 | MARKPKVADGQVIQYTTLKVRLYPTEAQAELFEKTFGCCRYIWNRMLADQRRFYEETGAHFIPTPAKY |
| | KNGAPFLKEVDNQALTQEYNKLAQAFRVFFKSPEVFRHPKFKRKKDDRDSFTACSHEFESGPTIYTTRD |
| | GIRMTKAGIVKAKFSRRPQAWWKLKRITVSKTKAGKYYCSILYDCPVKKPEPVVPTPETTLGLKYSMGH |
| | FYVADNGEMAGPPRWLKQSREKLVRIQQKLSRMEPGSRNYEQAVQKYRLLHEHIANQRRDFLHKESSR |
| | IANGWDAVCMRDDDMREMSQKVMLGNALEAGFGTFRELLRYKLERQGKSLVLLDRYTPTTRTCSVCA |
| | MVQSGVDYTASAWTCPKCGTIHNREVNAAKNIKLEGLARLCA |
| 490 | MSSREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKESGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 491 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPESFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |
| 492 | MKVRLYPSVNQKELFQKTFGCCRYIWNQMLSDHERFYLETDVHFIPTPAKYKKSAPFLSKVDNQALIQE |
| | HNKLSQAFRNFFRNPGAFGYPRFKRKKDDRDTFTACNQFFGRSATIYITQNAVRMTKVGLVRAVFPRRP |
| | RSGWRLTRITVERTRTDKYYGYLLYACPVRPPQPVTPTEETTVGLNYSVSRFYVADDGTAADPPRWLRQ |
| | SQDKLCQIQRQLCRMQKGSKNYQEMVQKYRLLHEHIANQRRDFLHKESRRIANEWDAVCVRSDSLTAL |
| | AAKTGGGCILDTGFGMFREMLRYKLERQGKSLLLVDRFRPTTKVCSVCGYVNEDLPAEALRWRCPVCG |
| | TEHRRERNAAANVKAIGLGRYRTETAAGGIG |
| 493 | MSSREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGTMTDTLIQAGSAVKESGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 494 | MNRAVKIRIYPNKEQRVQIEQTIGCSRFIYNQMLADKISYYQKEKKMLRNTPAGYKKEYPWLKEVDSLA |
| | LANAQLHLESAFRKFFREPACGFPRYKSKKHVRNSYTTNALNGNILLQDTHLKLPKMSVIRIKLHRQIPS |
| | DWKLKSVTVSREPSGKYFASLLFCCEDQTVEKRPAERFLGIDFAMQGMCVFSTGERAGYPMFYRKAEK |
| | KLAREQRKLSHCEKESRNYQKQKKRAALCHEKIKNQRKDFQHKLSRELAERYDAVCVEDLNLKGMSG |
| | GLHLGKGVQDNGYGQFLFMLGYKLEECGKHLIKVDRYFASSKICSVCGHKKKELALSDRMYVCECGN |
| | RMDRYVNAAVNIREEGKRIYKECA |
| 495 | MGHRETVGQAIQYNTIKVRLYPSVNQKELFQKTFGCCRYIWNQMLSDHERFYLETDVHFIPTPAKYKKS |
| | APFLSKVDNQALIQEHNKLSQAFRNFFRNPGAFGYPRFKRKKDDRDTFTACNQFFGRSATIYITQNAVR |
| | MTKVGLVRAVFPRRPRSGWRLTRITVERTRTDKYYGYLLYACPVRPPQPVTPTEETTVGLNYSVSRFYV |
| | ADDGTAADPPRWLRQSQDKLCQIQRQLCRMQKGSKNYQEMVQKYRLLHEHIANQRRDFLHKESRRIA |
| | NEWDAVCVRSDSLTALAAKTGGGCILDTGFGMFREMLRYKLERQGKSLLLVDRFRPTTKVCSVCGYVN |
| | EDLPAEALRWRCPVCGTEHRRERNAAANVKAIGLGRYRTETAAGGIG |
| 496 | MGHRETVGQAIQYNTIKVRLYPSVNQKELFQKTFGCCRYIWNQMLSDHERFYLETDVHFIPTPAKYKKS |
| | APFLSKVDNQALIQEHNKLSQAFRNFFRNPGAFGYPRFKRKKDDRDTFTACNQFFGRSATIYITQNAVR |
| | MTKVGLVRAVFPRRPRSGWRLTRITVERTRTDKYYGYLLYACPVRPPQPVTPTEETTVGLNYSVSRFYV |
| | ADDGTAADPPRWLRQSQDQLCQIQRQLCRMQKGSKNYQEMVQKYRLLHEHIANQRRDFLHKESRRIA |
| | NEWDAVCVRSDSLTALAAKTGGGCILDTGFGMFREMLRYKLERQGKSLLLVDRFRPTTKVCSVCGYVN |
| | EDLPAEALRWRCPVCGTEHRRERNAAANVKAIGLGRYRTETAAGGIG |
| 497 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSTVKEAGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 498 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 499 | MYGKGAARKGGKTQYTTIKVRLEPTAEQAELFEKTFGCCRYIWNQMLADQQRFYAETDAHFIPTPAKY |
| | KKEAPFLKEVDNQALIQEHNKLSQAFRVFFKNPESFGYPHFKRKKNDRDSFTACNHVFESGPTIYLTKNG |
| | IRMTKAGIVRARFHRRPQNGWDLKRITVEKTRAGKYYCCILYAYAAEEPEPVVPAPETTVGLNYSVSHF |
| | YAADDGSTADPPRWMKQSQEKLVRLQRRLSRMQPGSQNYREAVRKYRLLHEHIANQRLDFVHKESRRI |
| | ANAWEAVCVRGDDLGDIARKLVYGNALESGYGMFRECLRYKLERQGKPLIVVDRYAPTARTCSACGL |
| | VRDAVGLKEDLWTCPKCGAAHRREVNAAKNIKAQGLARYFGSQERRVSA |
| 500 | MAAKRSKSETLRYTTLKVRLYPSAEQAALFEKTFGCCRYIWNQMLADQQRFYIETDKFFIPTPAKYKAG |
| | APFLKEVDNQALIQEHNKLGQAFRVFFKSPENFGYPKFKRKKDDRDSFTVCNHVMGNSETVYTTRDGL |
| | RMTKAGIVRAKFPRRPQGWWKLKRVTVDRTRSGKYYGYILYECPEKKPEVVVPTPETTVGLKYSMARF |
| | YVADTGETADPPHWLKQSQEKLARIQQRLNRMRPGSKNYQETVQKYRLLHEHIANQRRDFIHKESRRIA |
| | NAWDAVCVRGDDMEQISRITNRGNALEAGFGMFRECLRYKLARQGKELLVVDRYFPSTRTCSACGRV |
| | MPEEISMKRRTWTCPQCGAVLKREANAARNIKDQGLAQYFSTRERRESA |
| 501 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |
| 502 | MSSREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 503 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 504 | MVGRSQSSHVQAGKTSLYTTIKARLYPTAEQAELFEKTFGCCRFIWNRMLSDQQKFYDETGAHFIPTPA |
| | KYKDGAPFLKEVDNQALIQTHNQLSQAFRIFFKNPEHFGHPRFKRKKDGRDAFTACNHVFSSGPTIYLTR |
| | DGIRMTKAGVVKAKFPRRPRNGWKLKRITVSKTRTGTYNCSIVFEYPAPAPQPIPPTPERTIGLKYSVSHF |
| | YVADNGAMADPPHWLKLTQEKLARLQQRMARMTPGSRNYEEAVQKYRLLHEHIANQRRDYIHKESRR |
| | IANAWDAVCVRADDLADGNRAMKLSNGLELGFGMFRACLDYKLSRQGKSLLMVERCAPTSRQCHSCG |
| | YLLPEGVDYRREQWRCPACGAALQREINAAQNIKTAGLRQVLTTQKSA |
| 505 | MSTETRKSRYTVLKVPAYPTPEQAQLMEKTFGCCRYLWNQMLSDVQEFYAATDIHYIPTPARYKKQAP |
| | FLKEVDSQALCAVHQSLRKAYLDFFRNPKVFQYPKPKTKKARKDSFTVYCRPYHTGPSLRLTDAGLQM |
| | PKLGLLQVRLYRKPLHWWSLRSVTMTKTKTGKYFCSITFGYEAELPEPVIPTPARTVGLNYSMSRFYVD |
| | SNGHSPELPPQMAAAREKLARMQRKLSRMQQGSKNYEAQLHKIRLQYERIANQRRDFAHQQSRRIANA |
| | WDAVCVRGDDLNVMAQRLKGGNVPDSGFGMFRAFLRYKLEAQGKAYIDVDPYAPAAKTCHACGHVN |
| | ENLPARARSWVCPLCGEELLREENTAQNIRDFGLMAVTRQPGVA |
| 506 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSTVKEAGFGMFREMLCYKLARQGKAFIQVDRYLPTTRSCSACGLTRDA |
| | LHARDYRRSGWVCPECGAVHDREVNAAKNIKARGLEQFFDLQGQDRSA |
| 507 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQPPEPVLPAPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |
| 508 | MASREKQYNVLKLRLYPTSEQAELFEKTFGCCRYLWNQMLADQQRFYLETGVHFIPTPAKYKKGAPFL |
| | KEVDNQALIQEHNQLSRAFRLFFQNPEAFGHPNFKRKKDDRDSFTACNHVFTSGPTIYTTRDGIRMTKAG |
| | MIRAVFPRRPQNGWKLKRVTVEKARTGRYYAYVLYESLVQSPEPVLPVPERTLGLKYSLRHFYVDDQG |
| | NRADPPRWLKQSQEKLVHLQRRLNRMQPGSKNYEEAVLKYRLLHEHIANQRRDFLHKESRRIANAWD |
| | AVCVRGDDLGAMTDTLIQAGSAVKEAGFGMFREMLCYKLARQGKAFIQVDRYLPTTRSCSACGLTRDA |
| | LHARDYRRSGWVCPECGAVHDREVNAAKNIKARGLEQFFDLQGQNRSA |

fliC-associated TldRs

| | Predicted | Predicted ωRNA | Predicted |
|---|---|---|---|
| TldR | ωRNA | right end | guide |
| SEQ ID NO | SEQ ID NO | SEQ ID NO | SEQ ID NO |
| 1 | 509 | 598 | 687 |
| 1 | 510 | 599 | 688 |
| 3 | 511 | 600 | 689 |
| 8 | 512 | 601 | 690 |
| 11 | 513 | 602 | 691 |
| 11 | 514 | 603 | 692 |
| 13 | 515 | 604 | 693 |
| 13 | 516 | 605 | 694 |
| 13 | 517 | 606 | 695 |
| 15 | 518 | 607 | 696 |
| 16 | 519 | 608 | 697 |
| 16 | 520 | 609 | 698 |
| 17 | 521 | 610 | 699 |
| 17 | 522 | 611 | 700 |
| 17 | 523 | 612 | 701 |
| 20 | 524 | 613 | 702 |
| 21 | 525 | 614 | 703 |
| 22 | 526 | 615 | 704 |
| 22 | 527 | 616 | 705 |
| 23 | 528 | 617 | 706 |
| 23 | 529 | 618 | 707 |
| 24 | 530 | 619 | 708 |
| 24 | 531 | 620 | 709 |
| 32 | 532 | 621 | 710 |
| 34 | 533 | 622 | 711 |
| 35 | 534 | 623 | 712 |
| 36 | 535 | 624 | 713 |
| 37 | 536 | 625 | 714 |
| 45 | 537 | 626 | 715 |
| 46 | 538 | 627 | 716 |
| 49 | 539 | 628 | 717 |
| 49 | 540 | 629 | 718 |
| 54 | 541 | 630 | 719 |
| 56 | 542 | 631 | 720 |
| 57 | 543 | 632 | 721 |
| 58 | 544 | 633 | 722 |
| 59 | 545 | 634 | 723 |
| 60 | 546 | 635 | 724 |
| 61 | 547 | 636 | 725 |
| 62 | 548 | 637 | 726 |
| 72 | 549 | 638 | 727 |
| 72 | 550 | 639 | 728 |
| 73 | 551 | 640 | 729 |
| 74 | 552 | 641 | 730 |
| 75 | 553 | 642 | 731 |
| 79 | 554 | 643 | 732 |
| 86 | 555 | 644 | 733 |
| 87 | 556 | 645 | 734 |
| 95 | 557 | 646 | 735 |
| 100 | 558 | 647 | 736 |
| 104 | 559 | 648 | 737 |
| 106 | 560 | 649 | 738 |
| 111 | 561 | 650 | 739 |
| 113 | 562 | 651 | 740 |
| 235 | 563 | 652 | 741 |
| 240 | 564 | 653 | 742 |
| 241 | 565 | 654 | 743 |
| 243 | 566 | 655 | 744 |
| 243 | 567 | 656 | 745 |
| 254 | 568 | 657 | 746 |
| 269 | 569 | 658 | 747 |
| 275 | 570 | 659 | 748 |
| 283 | 571 | 660 | 749 |
| 285 | 572 | 661 | 750 |
| 291 | 573 | 662 | 751 |
| 292 | 574 | 663 | 752 |
| 293 | 575 | 664 | 753 |
| 296 | 576 | 665 | 754 |
| 297 | 577 | 666 | 755 |
| 312 | 578 | 667 | 756 |
| 342 | 579 | 668 | 757 |
| 344 | 580 | 669 | 758 |
| 369 | 581 | 670 | 759 |
| 370 | 582 | 671 | 760 |
| 371 | 583 | 672 | 761 |
| 372 | 584 | 673 | 762 |
| 373 | 585 | 674 | 763 |
| 374 | 586 | 675 | 764 |
| 375 | 587 | 676 | 765 |
| 378 | 588 | 677 | 766 |
| 379 | 589 | 678 | 767 |
| 380 | 590 | 679 | 768 |
| 381 | 591 | 680 | 769 |
| 384 | 592 | 681 | 770 |
| 385 | 593 | 682 | 771 |
| 386 | 594 | 683 | 772 |
| 387 | 595 | 684 | 773 |
| 389 | 596 | 685 | 774 |
| 390 | 597 | 686 | 775 |

oppF-associated TldRs

| TldR | Predicted ωRNA | Predicted guide |
|---|---|---|
| SEQ ID NO | SEQ ID NO | SEQ ID NO |
| 114 | 776 | 1081 |
| 116 | 777 | 1082 |
| 119 | 778 | 1083 |
| 119 | 779 | 1084 |
| 120 | 780 | 1085 |
| 121 | 781 | 1086 |
| 121 | 782 | 1087 |
| 122 | 783 | 1088 |
| 123 | 784 | 1089 |
| 124 | 785 | 1090 |
| 125 | 786 | 1091 |
| 126 | 787 | 1092 |
| 127 | 788 | 1093 |
| 130 | 789 | 1094 |
| 130 | 790 | 1095 |
| 130 | 791 | 1096 |
| 131 | 792 | 1097 |
| 132 | 793 | 1098 |
| 134 | 794 | 1099 |
| 136 | 795 | 1100 |
| 137 | 796 | 1101 |
| 140 | 797 | 1102 |
| 141 | 798 | 1103 |
| 143 | 799 | 1104 |
| 143 | 800 | 1105 |
| 143 | 801 | 1106 |
| 143 | 802 | 1107 |
| 143 | 803 | 1108 |
| 143 | 804 | 1109 |
| 143 | 805 | 1110 |
| 143 | 806 | 1111 |
| 143 | 807 | 1112 |
| 143 | 808 | 1113 |
| 143 | 809 | 1114 |
| 148 | 810 | 1115 |
| 148 | 811 | 1116 |
| 149 | 812 | 1117 |
| 150 | 813 | 1118 |
| 150 | 814 | 1119 |
| 150 | 815 | 1120 |
| 150 | 816 | 1121 |
| 150 | 817 | 1122 |
| 150 | 818 | 1123 |
| 150 | 819 | 1124 |
| 151 | 820 | 1125 |
| 151 | 821 | 1126 |
| 151 | 822 | 1127 |
| 151 | 823 | 1128 |
| 151 | 824 | 1129 |
| 151 | 825 | 1130 |
| 151 | 826 | 1131 |
| 151 | 827 | 1132 |
| 151 | 828 | 1133 |
| 152 | 829 | 1134 |
| 153 | 830 | 1135 |
| 153 | 831 | 1136 |
| 153 | 832 | 1137 |
| 154 | 833 | 1138 |
| 155 | 834 | 1139 |
| 156 | 835 | 1140 |
| 156 | 836 | 1141 |
| 158 | 837 | 1142 |
| 163 | 838 | 1143 |
| 165 | 839 | 1144 |
| 166 | 840 | 1145 |
| 167 | 841 | 1146 |
| 168 | 842 | 1147 |
| 169 | 843 | 1148 |
| 170 | 844 | 1149 |
| 170 | 845 | 1150 |
| 170 | 846 | 1151 |
| 170 | 847 | 1152 |
| 172 | 848 | 1153 |
| 173 | 849 | 1154 |
| 174 | 850 | 1155 |
| 175 | 851 | 1156 |
| 175 | 852 | 1157 |
| 175 | 853 | 1158 |
| 177 | 854 | 1159 |
| 177 | 855 | 1160 |
| 178 | 856 | 1161 |
| 178 | 857 | 1162 |
| 178 | 858 | 1163 |
| 182 | 859 | 1164 |
| 185 | 860 | 1165 |
| 189 | 861 | 1166 |
| 190 | 862 | 1167 |
| 191 | 863 | 1168 |
| 194 | 864 | 1169 |
| 195 | 865 | 1170 |
| 196 | 866 | 1171 |
| 197 | 867 | 1172 |
| 198 | 868 | 1173 |
| 199 | 869 | 1174 |
| 200 | 870 | 1175 |
| 202 | 871 | 1176 |
| 206 | 872 | 1177 |
| 206 | 873 | 1178 |
| 207 | 874 | 1179 |
| 208 | 875 | 1180 |
| 209 | 876 | 1181 |
| 210 | 877 | 1182 |
| 211 | 878 | 1183 |
| 212 | 879 | 1184 |
| 213 | 880 | 1185 |
| 214 | 881 | 1186 |
| 216 | 882 | 1187 |
| 217 | 883 | 1188 |
| 218 | 884 | 1189 |
| 219 | 885 | 1190 |
| 220 | 886 | 1191 |
| 220 | 887 | 1192 |
| 220 | 888 | 1193 |
| 221 | 889 | 1194 |
| 223 | 890 | 1195 |
| 224 | 891 | 1196 |
| 225 | 892 | 1197 |
| 226 | 893 | 1198 |
| 391 | 894 | 1199 |
| 391 | 895 | 1200 |
| 391 | 896 | 1201 |
| 391 | 897 | 1202 |
| 391 | 898 | 1203 |
| 391 | 899 | 1204 |
| 391 | 900 | 1205 |
| 391 | 901 | 1206 |
| 391 | 902 | 1207 |
| 391 | 903 | 1208 |
| 391 | 904 | 1209 |
| 391 | 905 | 1210 |
| 391 | 906 | 1211 |
| 391 | 907 | 1212 |
| 391 | 908 | 1213 |
| 391 | 909 | 1214 |
| 391 | 910 | 1215 |
| 391 | 911 | 1216 |
| 391 | 912 | 1217 |
| 391 | 913 | 1218 |
| 391 | 914 | 1219 |
| 391 | 915 | 1220 |
| 391 | 916 | 1221 |
| 391 | 917 | 1222 |
| 391 | 918 | 1223 |
| 391 | 919 | 1224 |
| 391 | 920 | 1225 |
| 391 | 921 | 1226 |
| 391 | 922 | 1227 |
| 391 | 923 | 1228 |
| 391 | 924 | 1229 |
| 391 | 925 | 1230 |
| 391 | 926 | 1231 |
| 391 | 927 | 1232 |
| 391 | 928 | 1233 |
| 391 | 929 | 1234 |
| 391 | 930 | 1235 |
| 391 | 931 | 1236 |
| 391 | 932 | 1237 |
| 391 | 933 | 1238 |
| 391 | 934 | 1239 |
| 391 | 935 | 1240 |
| 391 | 936 | 1241 |
| 391 | 937 | 1242 |
| 391 | 938 | 1243 |
| 392 | 939 | 1244 |
| 392 | 940 | 1245 |
| 392 | 941 | 1246 |
| 392 | 942 | 1247 |
| 392 | 943 | 1248 |
| 392 | 944 | 1249 |
| 392 | 945 | 1250 |
| 392 | 946 | 1251 |
| 392 | 947 | 1252 |
| 392 | 948 | 1253 |
| 392 | 949 | 1254 |
| 393 | 950 | 1255 |
| 394 | 951 | 1256 |
| 394 | 952 | 1257 |
| 394 | 953 | 1258 |
| 394 | 954 | 1259 |
| 394 | 955 | 1260 |
| 394 | 956 | 1261 |
| 394 | 957 | 1262 |
| 395 | 958 | 1263 |
| 395 | 959 | 1264 |
| 395 | 960 | 1265 |
| 396 | 961 | 1266 |
| 397 | 962 | 1267 |
| 397 | 963 | 1268 |
| 398 | 964 | 1269 |
| 398 | 965 | 1270 |
| 398 | 966 | 1271 |
| 398 | 967 | 1272 |
| 398 | 968 | 1273 |
| 398 | 969 | 1274 |
| 398 | 970 | 1275 |
| 398 | 971 | 1276 |
| 398 | 972 | 1277 |
| 398 | 973 | 1278 |
| 399 | 974 | 1279 |
| 400 | 975 | 1280 |
| 401 | 976 | 1281 |
| 401 | 977 | 1282 |
| 401 | 978 | 1283 |
| 401 | 979 | 1284 |
| 401 | 980 | 1285 |
| 401 | 981 | 1286 |
| 402 | 982 | 1287 |
| 402 | 983 | 1288 |
| 402 | 984 | 1289 |
| 402 | 985 | 1290 |
| 402 | 986 | 1291 |
| 402 | 987 | 1292 |
| 402 | 988 | 1293 |
| 402 | 989 | 1294 |
| 403 | 990 | 1295 |
| 403 | 991 | 1296 |
| 403 | 992 | 1297 |
| 403 | 993 | 1298 |
| 403 | 994 | 1299 |
| 403 | 995 | 1300 |
| 403 | 996 | 1301 |
| 404 | 997 | 1302 |
| 404 | 998 | 1303 |
| 405 | 999 | 1304 |
| 406 | 1000 | 1305 |
| 407 | 1001 | 1306 |
| 407 | 1002 | 1307 |
| 408 | 1003 | 1308 |
| 408 | 1004 | 1309 |
| 409 | 1005 | 1310 |
| 410 | 1006 | 1311 |
| 411 | 1007 | 1312 |
| 411 | 1008 | 1313 |
| 412 | 1009 | 1314 |
| 412 | 1010 | 1315 |
| 413 | 1011 | 1316 |
| 414 | 1012 | 1317 |
| 415 | 1013 | 1318 |
| 415 | 1014 | 1319 |
| 416 | 1015 | 1320 |
| 417 | 1016 | 1321 |
| 418 | 1017 | 1322 |
| 419 | 1018 | 1323 |
| 419 | 1019 | 1324 |
| 420 | 1020 | 1325 |
| 421 | 1021 | 1326 |
| 422 | 1022 | 1327 |
| 423 | 1023 | 1328 |
| 424 | 1024 | 1329 |
| 425 | 1025 | 1330 |
| 426 | 1026 | 1331 |
| 426 | 1027 | 1332 |
| 426 | 1028 | 1333 |
| 426 | 1029 | 1334 |
| 426 | 1030 | 1335 |
| 427 | 1031 | 1336 |
| 428 | 1032 | 1337 |
| 429 | 1033 | 1338 |
| 429 | 1034 | 1339 |
| 430 | 1035 | 1340 |
| 431 | 1036 | 1341 |
| 432 | 1037 | 1342 |
| 433 | 1038 | 1343 |
| 434 | 1039 | 1344 |
| 435 | 1040 | 1345 |
| 436 | 1041 | 1346 |
| 437 | 1042 | 1347 |
| 438 | 1043 | 1348 |
| 439 | 1044 | 1349 |
| 440 | 1045 | 1350 |
| 441 | 1046 | 1351 |
| 442 | 1047 | 1352 |
| 443 | 1048 | 1353 |
| 444 | 1049 | 1354 |
| 445 | 1050 | 1355 |
| 445 | 1051 | 1356 |
| 446 | 1052 | 1357 |
| 447 | 1053 | 1358 |
| 448 | 1054 | 1359 |
| 449 | 1055 | 1360 |
| 450 | 1056 | 1361 |
| 451 | 1057 | 1362 |
| 452 | 1058 | 1363 |
| 453 | 1059 | 1364 |
| 453 | 1060 | 1365 |
| 454 | 1061 | 1366 |
| 455 | 1062 | 1367 |
| 456 | 1063 | 1368 |
| 457 | 1064 | 1369 |
| 458 | 1065 | 1370 |
| 459 | 1066 | 1371 |
| 460 | 1067 | 1372 |
| 461 | 1068 | 1373 |
| 462 | 1069 | NNNNNNNNNNNNNNN |
| | | NNNNN |
| 463 | 1070 | 1374 |
| 464 | 1071 | 1375 |
| 465 | 1072 | 1376 |
| 466 | 1073 | 1377 |
| 467 | 1074 | 1378 |
| 468 | 1075 | 1379 |
| 469 | 1076 | 1380 |
| 470 | 1077 | 1381 |
| 471 | 1078 | 1382 |
| 472 | 1079 | 1383 |
| 477 | 1080 | 1384 |

TnpB-transposase fusion sequences

| | Fusion | | | | |
|---|---|---|---|---|---|
| Transposase | protein | | SEQ | HMM | |
| domain | accession | Fusion protein sequence | ID NO | description | Organism |
| Crinkler | KAG9062067.1 | MDLADLKGIRVSGGIVVTSQDLVED | 1453 | Crinkler | Linnemannia |
| | | AGRPILQDRTFTAIWRFYKYTTGKR | | effector | hyalina |
| | | VKDSFWHISNWIPTRPIEQVPVLTRG | | protein N- | |
| | | WRTEEFAPETSTKSPPKPKTNVEKAP | | terminal | |
| | | ASAKKPLAEMKKECFRLAAGKKRE | | domain | |
| | | AQRLIGIFVETLRIRTDSAEEALRIKL | | | |
| | | PPGKLTVSEEQRTKARRGAASGTER | | | |
| | | EIFDHLCERIKPKDYVEDDDEDATD | | | |
| | | KKRENNSDLRDLKGFGARELLPKD | | | |
| | | KDDDRDKDKGKKEKTSLGIVVNYLI | | | |
| | | DWLVTGHFYKPSRRRGEIEVKMPYT | | | |
| | | PTYVVRSVAGQLAVELKKLYGNGSH | | | |
| | | ELRKKVLTVHKKGVLDASIDIVIQE | | | |
| | | QVSAFENFLFLNKLTSSSRRIVPLTTS | | | |
| | | HQPFVSFSERIDQQTEEEGWTSTSCL | | | |
| | | DALDDIRSHLQQFLQEDEKVKKNIK | | | |
| | | DDDEGVFHWAMYKEKGYVLRGSIL | | | |
| | | TDGFRVKLQSFKLRELQDVRYRRW | | | |
| | | KEDRLPSRLTSTVGGIDFFLQEIRNV | | | |
| | | LTCKEDIERLWPGVDVKDIRTLTLDA | | | |
| | | GQACIIGAFAHLPEEIAKRARSLGYL | | | |
| | | VVGLNEYYTSKKCPRCGQFVGQVD | | | |
| | | MRRFYCSQFQVFHHRDVMAAENM | | | |
| | | ANIVQGYLLDLQRLDCLHPIAPDGN | | | |
| | | MPWKEASSGLGTPSTTGPTATKIAAT | | | |
| | | SGPTRVALRSKGQRKRSSTASSLKQ | | | |
| | | ATSNQSLWCIVDGDPMLRAFELVIPS | | | |
| | | SVTTLGQLRSYIHLRKPIWFKYLEAE | | | |
| | | DLTLWSVSIPITKDNEDTPILLEDVPS | | | |
| | | SDKNKLGPTDDVSELFQQVPLKKTI | | | |
| | | HVIVQRPPPAVTMKRLLEQDPQYLP | | | |
| | | QKKRIRIEEGWKPFTASDGILVDLPP | | | |
| | | YWIDILASTEFVPKPRAAFDHLKGN | | | |
| | | LQAGDAIIVPSMGQNPKDFGLYGQD | | | |
| | | HNLFVTEQMLELWDEMRGDQEFTY | | | |
| | | RRILSGPMGVGKSYLSYFLAARAYA | | | |
| | | EGWLVLYISDAGVLDKNKQDESAL | | | |
| | | QAVKRFLALNKDILTGAEMEMLVN | | | |
| | | DYNGTDDISGNAMSVIFGTLLKSRD | | | |
| | | RKTLLLVDEHGKLFEKEPYVPDKFR | | | |
| | | SLVPLLLYNWWGEDAKGSRVVFTG | | | |
| | | TAHAKYEMGILEESYRFTSLVLVGPL | | | |
| | | SMHVFSKLLDMYPRLAAPAIRKEVT | | | |
| | | AITNCVPRELVHLSVYLELFPDPIAID | | | |
| | | NLQVWTSKRTKVFLSTAKTYYESRT | | | |
| | | PFRKNDFYEALVHTFLGSTSIVDFE | | | |
| | | WDFLDLGLIYRCKDVGRIGTHHHIL | | | |
| | | CRPAQRALLELFKNISLPDAIKKRIC | | | |
| | | DGSLDENEFEGVLYHQLICATKPIVL | | | |
| | | GTTDLNGKNPDTISLDFSLCETLRAG | | | |
| | | MTCLESDHEMALTRGYDSHPRFDF | | | |
| | | MLGPMFMQVSVSDFGKHNTASAEL | | | |
| | | GKAFNDRDNNGTNQIERYLNDLYGP | | | |
| | | GHSAKIDNSKFIVTKNGVDVLGFRI | | | |
| | | VYICGSPGQPSHSKWVKKFPDVRHV | | | |
| | | SFEEVKENLFKNIVTSTVAIAPMTT | | | |
| DDE_3 | XP_052966910.1 | MTIRHTSRVPARRSTFFEQACPVIRT | 1454 | DDE | Polychytrium |
| | | LIEQDPFQTGPVLQCKLESILQRKVS | | superfamily | aggregatum |
| | | LSLCHTAIQRAGLSHKRATHLYKSK | | endonuclease | |
| | | RLDERVAQFRDQVRSIDPRRFVFVD | | | |
| | | ETGIRKSIFPLYGYSPKGAPLRQCNH | | | |
| | | MKHKSVSAAFAINHSGVLHRMYIPT | | | |
| | | SFKTHSMVEFFERATFPDDSVVVMD | | | |
| | | NVSMHKTRAVLDCIERRGWSVIFTP | | | |
| | | PASPDFNPIENFFGVVKHQYRKAAA | | | |
| | | LNASEGFKVGMVTLFEVTSPTSPQR | | | |
| | | RAPALGHPAFSGPMPPKRKGTGDGQ | | | |
| | | PRPPRPKKAKSQSEPVSDPMDVDPT | | | |
| | | EPGPSTSSSTGKRCRQCDGTDHERC | | | |
| | | NTSKCPKYRPLKSSVVPLVDGRSSA | | | |
| | | PQFSCFKQTMDGCCLNPALRDKIQA | | | |
| | | TVEAMTQIQFEASRLLNLHILRCIEQ | | | |
| | | DLPVPVISKATPAFIRQCFTIVEAGAL | | | |
| | | SPTGDNEKYNEHLVASFRDYQTTRH | | | |
| | | ADLPPAPRLPDGAHSQLLTLAVNAY | | | |
| | | ATNCTTHLNLAYWRLLRRFCAAVFP | | | |
| | | ANKHKHEAIEACLELFEKPGQTPPK | | | |
| | | ADLEFLYPDFGHVLDEFRYSNDQDR | | | |
| | | FRALHRLSHHVRILARDKMVDDVH | | | |
| | | KWIDCDMPALRVTELQRQAKLDELT | | | |
| | | TQGIPSDDKRFTTAKSELGTTKAKIN | | | |
| | | KLASRINWALKQLALDTPPAVCTIVP | | | |
| | | LCTAKVKYVKIDTVTLWQLLDTEQ | | | |
| | | HLGLTWDMLKEEGEDGINNQDRLW | | | |
| | | RSCFKLRSSLFQERDVNKKLFNYEIS | | | |
| | | TDGIGCTIGLVKFVRRTDHTETTTPV | | | |
| | | KTAEQRIQAHIGSPDPDKTTWIGIDP | | | |
| | | GVGSIFTAVIQAPGHQDEVVSFSNGH | | | |
| | | YQHMCGHKSNTDWHNTMKYRLSIE | | | |
| | | TWMSTTPSPKVSSSEAFKVHLTHVL | | | |
| | | RPELRVQLDFHLGKKARHHRFTGH | | | |
| | | KRRQVAVDRMCKAVLKHAPIRTDV | | | |
| | | VIAFGNASWRQGKGYASSPRRQRFA | | | |
| | | RYFEQFEHHRRRPNHPHGSVKVAST | | | |
| | | NEFNTSQVCSKCLEPVRLEGLDAPS | | | |
| | | VANSHFVRSCKNPSCRTVWNRDVN | | | |
| | | AARNMITSTRIRSVKGQLRIRLKDYI | | | |
| | | TKQTKEIVPLSKEASERAKRFWEIVR | | | |
| | | KAFLRKDIDAFALLAGTKAPVPYPIT | | | |
| | | IRQGFQFKYRLRSKHAIVCAIQHPRV | | | |
| | | PIRFEGGCLGTEADAGYEESEDESSL | | | |
| | | EDDHDHPDGKESTKVKKSRPYTIAC | | | |
| | | VDRARGIIVWDVTRVSFSTKPRIML | | | |
| | | KLKTDLTNFVFLSKFGMYCGCSYD | | | |
| | | KSIKFFNARFELANIYYTMKAVQFV | | | |
| | | RYNSISHELVTAGSHNICPTLKYEIET | | | |
| | | DLSTDEWITSIYLDEANHRMYAIIDT | | | |
| | | RILICCILHYSEYNYTIIACADGSIKIK | | | |
| | | NLTNAVVHEFTSHTKRVTGLAVYPF | | | |
| | | GPIIMSCALDMTVRMYNLKNFKEV | | | |
| | | YCFHIREQPMGIEIADKAVLNIYTRE | | | |
| | | GILAWNLNHINTSFSSINSQAKRLVN | | | |
| | | YQGTRTPCRILAWSDDSVIRLISPATG | | | |
| | | KSITTVLPLVESESITALSYCPSIEKLF | | | |
| | | IMLTNSEIWVIATNVNPCLVVDIWRP | | | |
| | | NGPIREDCTCICVCDGVFQHHQPSPS | | | |
| | | GYERSKGFAFLFGGTGNGQVLVYTR | | | |
| | | FGVLHNGEVTQIIYISKQQLLITGGA | | | |
| | | DELIKICTLEPMSSELIQVKVSIKAGF | | | |
| | | IPRLISISDNAVAATSDDWSIHMFQFN | | | |
| | | LHRNESRKMPTHLRSDDHTDAVTAI | | | |
| | | CPIPMLGLFISSSRDGTLRLWDSFNT | | | |
| | | LIREVQFTQPLEAVCVNSERGDILVG | | | |
| | | IQNRVDIIQYSLYLPPGYIATVQSLEF | | | |
| | | PEMPVEPSLPFDDNQAIWKAVPLQS | | | |
| | | YSTRQDFFHAINLVSTGVEALSSQSL | | | |
| | | SSATTFELETPMLSKEHQLSSTENIY | | | |
| | | RMLDVLMLRRQEVVDRAKKRISEE | | | |
| | | LSNVRRKDQILHEEYEKYIKYRPLM | | | |
| | | QRELEEDSEKAGYRSYSLSYLQFSA | | | |
| | | PITPRGEVAEELTLLREVKEIQMNEA | | | |
| | | ESIESVAALEQAAESIQSVTLEEPSVP | | | |
| | | IIQEVPQESISQIIAKMPVTPKHRLKV | | | |
| | | APDGEIPNSVLSHNVESWRSKHTGY | | | |
| | | QRAGAIRGIRRKKKEAPKPADTVER | | | |
| | | KKKSDEYKERLKNLLENMAKKEEE | | | |
| | | EKAQANEANIVVIEDEDENEAEEEE | | | |
| | | EVYRSRLPANTGLRNVQMYDPIVQ | | | |
| | | VVAEKVPMIVQKALAFSWFPDDEKI | | | |
| | | NPTPEAIAEIIVGKLWEYSKPEIKLE | | | |
| | | MLDFINWIYEELGIRDTTMIMRTLCR | | | |
| | | YLQSRLTESMDDTDVKLREKIIGVLT | | | |
| | | KFSVPYTEVISTLIMQLILPYEPIVAPS | | | |
| | | KHLMSALGIACAESQFLKMQIEEIY | | | |
| | | NESQNQLMAFNAARGESGSRPQTLT | | | |
| | | KPKDFRELATLWIRTCLKNYLMKTL | | | |
| | | KDKDAIALLKKLTPFGIEDRGSTSNS | | | |
| | | SAPSTPAQASSTGTAKTSNPSTPSNQI | | | |
| | | DKSRRSSVKGRRESTATQSRRPTSPQ | | | |
| | | KTRSRQTSIATGANQGRPRASSVSFH | | | |
| | | PDVGESGPEDPKDQLRRLAVTLESS | | | |
| | | NEKLDEETNEAIVSRPVTAIGDSTIE | | | |
| | | GVVIETRDPGSRDPVSILRNPSGQDF | | | |
| | | VDAVNYFIVTLEKKAAKEEQERLAK | | | |
| | | LREQSAQAQRQRLEAEKKAQLEEY | | | |
| | | LRQKEEERQARSAARRERIASLRAQ | | | |
| | | EKAKADESQKRRRGVNWKTVGQT | | | |
| | | HQSKCHPSRETLNVALDKFPSMCGS | | | |
| | | FLRSFTNEITLNMASLAKTMPMEHV | | | |
| | | VLSPFGEPPLSQRILSAAAREKVHGR | | | |
| | | DYPYSPFVNSQHTLVPEWKEYHAD | | | |
| | | ERRSSTTRSDVSYIRAPLTFRENNVR | | | |
| | | IVKDLYLQEFDPEGDESAKFKTQKK | | | |
| | | YFIPSLAVPDIDDELNDELQNMDESE | | | |
| | | RPSQAHSHRHSHSHSHSQSFHRLSET | | | |
| | | DQQLQRMKNRRYI | | | |
| DDE_3 | XP_052966910.1 | MTIRHTSRVPARRSTFFEQACPVIRT | 1455 | DDE | Polychytrium |
| | | LIEQDPFQTGPVLQCKLESILQRKVS | | superfamily | aggregatum |
| | | LSLCHTAIQRAGLSHKRATHLYKSK | | endonuclease | |
| | | RLDERVAQFRDQVRSIDPRRFVFVD | | | |
| | | ETGIRKSIFPLYGYSPKGAPLRQCNH | | | |
| | | MKHKSVSAAFAINHSGVLHRMYIPT | | | |
| | | SFKTHSMVEFFERATFPDDSVVVMD | | | |
| | | NVSMHKTRAVLDCIERRGWSVIFTP | | | |
| | | PASPDFNPIENFFGVVKHQYRKAAA | | | |
| | | LNASEGFKVGMVTLFEVTSPTSPQR | | | |
| | | RAPALGHPAFSGPMPPKRKGTGDGQ | | | |
| | | PRPPRPKKAKSQSEPVSDPMDVDPT | | | |
| | | EPGPSTSSSTGKRCRQCDGTDHERC | | | |
| | | NTSKCPKYRPLKSSVVPLVDGRSSA | | | |
| | | PQFSCFKQTMDGCCLNPALRDKIQA | | | |
| | | TVEAMTQIQFEASRLLNLHILRCIEQ | | | |
| | | DLPVPVISKATPAFIRQCFTIVEAGAL | | | |
| | | SPTGDNEKYNEHLVASFRDYQTTRH | | | |
| | | ADLPPAPRLPDGAHSQLLTLAVNAY | | | |
| | | ATNCTTHLNLAYWRLLRRFCAAVFP | | | |
| | | ANKHKHEAIEACLELFEKPGQTPPK | | | |
| | | ADLEFLYPDFGHVLDEFRYSNDQDR | | | |
| | | FRALHRLSHHVRILARDKMVDDVH | | | |
| | | KWIDCDMPALRVTELQRQAKLDELT | | | |
| | | TQGIPSDDKRFTTAKSELGTTKAKIN | | | |
| | | KLASRINWALKQLALDTPPAVCTIVP | | | |
| | | LCTAKVKYVKIDTVTLWQLLDTEQ | | | |
| | | HLGLTWDMLKEEGEDGINNQDRLW | | | |
| | | RSCFKLRSSLFQERDVNKKLFNYEIS | | | |
| | | TDGIGCTIGLVKFVRRTDHTETTTPV | | | |
| | | KTAEQRIQAHIGSPDPDKTTWIGIDP | | | |
| | | GVGSIFTAVIQAPGHQDEVVSFSNGH | | | |
| | | YQHMCGHKSNTDWHNTMKYRLSIE | | | |
| | | TWMSTTPSPKVSSSEAFKVHLTHVL | | | |
| | | RPELRVQLDFHLGKKARHHRFTGH | | | |
| | | KRRQVAVDRMCKAVLKHAPIRTDV | | | |
| | | VIAFGNASWRQGKGYASSPRRQRFA | | | |
| | | RYFEQFEHHRRRPNHPHGSVKVAST | | | |
| | | NEFNTSQVCSKCLEPVRLEGLDAPS | | | |
| | | VANSHFVRSCKNPSCRTVWNRDVN | | | |
| | | AARNMITSTRIRSVKGQLRIRLKDYI | | | |
| | | TKQTKEIVPLSKEASERAKRFWEIVR | | | |
| | | KAFLRKDIDAFALLAGTKAPVPYPIT | | | |
| | | IRQGFQFKYRLRSKHAIVCAIQHPRV | | | |
| | | PIRFEGGCLGTEADAGYEESEDESSL | | | |
| | | EDDHDHPDGKESTKVKKSRPYTIAC | | | |
| | | VDRARGIIVWDVTRVSFSTKPRIML | | | |
| | | KLKTDLTNFVFLSKFGMYCGCSYD | | | |
| | | KSIKFFNARFELANIYYTMKAVQFV | | | |
| | | RYNSISHELVTAGSHNICPTLKYEIET | | | |
| | | DLSTDEWITSIYLDEANHRMYAIIDT | | | |
| | | RILICCILHYSEYNYTIIACADGSIKIK | | | |
| | | NLTNAVVHEFTSHTKRVTGLAVYPF | | | |
| | | GPIIMSCALDMTVRMYNLKNFKEV | | | |
| | | YCFHIREQPMGIEIADKAVLNIYTRE | | | |
| | | GILAWNLNHINTSFSSINSQAKRLVN | | | |
| | | YQGTRTPCRILAWSDDSVIRLISPATG | | | |
| | | KSITTVLPLVESESITALSYCPSIEKLF | | | |
| | | IMLTNSEIWVIATNVNPCLVVDIWRP | | | |
| | | NGPIREDCTCICVCDGVFQHHQPSPS | | | |
| | | GYERSKGFAFLFGGTGNGQVLVYTR | | | |
| | | FGVLHNGEVTQIIYISKQQLLITGGA | | | |
| | | DELIKICTLEPMSSELIQVKVSIKAGF | | | |
| | | IPRLISISDNAVAATSDDWSIHMFQFN | | | |
| | | LHRNESRKMPTHLRSDDHTDAVTAI | | | |
| | | CPIPMLGLFISSSRDGTLRLWDSFNT | | | |
| | | LIREVQFTQPLEAVCVNSERGDILVG | | | |
| | | IQNRVDIIQYSLYLPPGYIATVQSLEF | | | |
| | | PEMPVEPSLPFDDNQAIWKAVPLQS | | | |
| | | YSTRQDFFHAINLVSTGVEALSSQSL | | | |
| | | SSATTFELETPMLSKEHQLSSTENIY | | | |
| | | RMLDVLMLRRQEVVDRAKKRISEE | | | |
| | | LSNVRRKDQILHEEYEKYIKYRPLM | | | |
| | | QRELEEDSEKAGYRSYSLSYLQFSA | | | |
| | | PITPRGEVAEELTLLREVKEIQMNEA | | | |
| | | ESIESVAALEQAAESIQSVTLEEPSVP | | | |
| | | IIQEVPQESISQIIAKMPVTPKHRLKV | | | |
| | | APDGEIPNSVLSHNVESWRSKHTGY | | | |
| | | QRAGAIRGIRRKKKEAPKPADTVER | | | |
| | | KKKSDEYKERLKNLLENMAKKEEE | | | |
| | | EKAQANEANIVVIEDEDENEAEEEE | | | |
| | | EVYRSRLPANTGLRNVQMYDPIVQ | | | |
| | | VVAEKVPMIVQKALAFSWFPDDEKI | | | |
| | | NPTPEAIAEIIVGKLWEYSKPEIKLE | | | |
| | | MLDFINWIYEELGIRDTTMIMRTLCR | | | |
| | | YLQSRLTESMDDTDVKLREKIIGVLT | | | |
| | | KFSVPYTEVISTLIMQLILPYEPIVAPS | | | |
| | | KHLMSALGIACAESQFLKMQIEEIY | | | |
| | | NESQNQLMAFNAARGESGSRPQTLT | | | |
| | | KPKDFRELATLWIRTCLKNYLMKTL | | | |
| | | KDKDAIALLKKLTPFGIEDRGSTSNS | | | |
| | | SAPSTPAQASSTGTAKTSNPSTPSNQI | | | |
| | | DKSRRSSVKGRRESTATQSRRPTSPQ | | | |
| | | KTRSRQTSIATGANQGRPRASSVSFH | | | |
| | | PDVGESGPEDPKDQLRRLAVTLESS | | | |
| | | NEKLDEETNEAIVSRPVTAIGDSTIE | | | |
| | | GVVIETRDPGSRDPVSILRNPSGQDF | | | |
| | | VDAVNYFIVTLEKKAAKEEQERLAK | | | |
| | | LREQSAQAQRQRLEAEKKAQLEEY | | | |
| | | LRQKEEERQARSAARRERIASLRAQ | | | |
| | | EKAKADESQKRRRGVNWKTVGQT | | | |
| | | HQSKCHPSRETLNVALDKFPSMCGS | | | |
| | | FLRSFTNEITLNMASLAKTMPMEHV | | | |
| | | VLSPFGEPPLSQRILSAAAREKVHGR | | | |
| | | DYPYSPFVNSQHTLVPEWKEYHAD | | | |
| | | ERRSSTTRSDVSYIRAPLTFRENNVR | | | |
| | | IVKDLYLQEFDPEGDESAKFKTQKK | | | |
| | | YFIPSLAVPDIDDELNDELQNMDESE | | | |
| | | RPSQAHSHRHSHSHSHSQSFHRLSET | | | |
| | | DQQLQRMKNRRYI | | | |
| DDE_Tnp_1 | WP_016084423.1 | MSLSIQEEFHLFAQELQQYLSPHILQ | 1456 | Transposase | Bacillus cereus |
| | | QLAQETGFVKRKSKYGARDLAALCI | | DDE domain | BAG1X2-1 |
| | | WISQHVASDSLTRLCSQLYANTATLM | | | |
| | | SPEGLNQRFNRCAVLFLQRVFSLLIK | | | |
| | | SKLNDFSQISNQYTSYFQRIRILDATI | | | |
| | | FQVPNHLAPIYPGSGGCAQTAGIKIQ | | | |
| | | LEYDLHSGKFLNFQMEPGKNNDKT | | | |
| | | FGTDCLDTLRPGDLCIRDLGYFSLK | | | |
| | | DLDQMDQRGVFYVSRLKLNNRVYV | | | |
| | | KNDYPEFFRDGTVKKQSLYVLLNLE | | | |
| | | DIMHQIKPGDTYENPKFFRSLEDKL | | | |
| | | AKAQRVLSRRLKGSSRWNKQRVKV | | | |
| | | SRIHEYISNTRKDYLDKISTEIIKNHD | | | |
| | | VIGIEDLHVSNMLKNHKLAKAISEV | | | |
| | | SWSQFRSMLEYKAKWYGKQVIVVS | | | |
| | | KTFASSQLCSCCGYQNKDVKNLNL | | | |
| | | RKWDCPSCCTHHDRDINASINLKNE | | | |
| | | AIRLLTARTAGLA | | | |
| DDE_Tnp_1 | WP_016084423.1 | MSLSIQEEFHLFAQELQQYLSPHILQ | 1457 | Transposase | Bacillus cereus |
| | | QLAQETGFVKRKSKYGARDLAALCI | | DDE domain | BAG1X2-1 |
| | | WISQHVASDSLTRLCSQLYANTATLM | | | |
| | | SPEGLNQRFNRCAVLFLQRVFSLLIK | | | |
| | | SKLNDFSQISNQYTSYFQRIRILDATI | | | |
| | | FQVPNHLAPIYPGSGGCAQTAGIKIQ | | | |
| | | LEYDLHSGKFLNFQMEPGKNNDKT | | | |
| | | FGTDCLDTLRPGDLCIRDLGYFSLK | | | |
| | | DLDQMDQRGVFYVSRLKLNNRVYV | | | |
| | | KNDYPEFFRDGTVKKQSLYVLLNLE | | | |
| | | DIMHQIKPGDTYENPKFFRSLEDKL | | | |
| | | AKAQRVLSRRLKGSSRWNKQRVKV | | | |
| | | SRIHEYISNTRKDYLDKISTEIIKNHD | | | |
| | | VIGIEDLHVSNMLKNHKLAKAISEV | | | |
| | | SWSQFRSMLEYKAKWYGKQVIVVS | | | |
| | | KTFASSQLCSCCGYQNKDVKNLNL | | | |
| | | RKWDCPSCCTHHDRDINASINLKNE | | | |
| | | AIRLLTARTAGLA | | | |
| DDE_Tnp_1 | WP_016083199.1 | MSLSIQEEFHLFAQELQQYLSPHILQ | 1458 | Transposase | Bacillus cereus |
| | | QLAQETGFVKRKSKYGARDLAALCI | | DDE domain | BAG1X1-1 |
| | | WISQHVASDSLTRLCSQLYANTATLM | | | |
| | | SPEGLNQRFNRCAVLFLQRVFSLLIK | | | |
| | | SKLNDFSQISNQYTSYFQRIRILDATI | | | |
| | | FQVPNHLAPIYPGSGGCAQTAGIKIQ | | | |
| | | LEYDLHSGKFLNFQMEPGKNNDKT | | | |
| | | FGTDCLDTLRPGDLCIRDLGYFSLK | | | |
| | | DLDQMDQRGVFYVSRLKLNNRVYV | | | |
| | | KNDYPEFFRDGTVKKQSLYVLLNLE | | | |
| | | DIMHQIKPGDTYENPKFFRSLEDKL | | | |
| | | AKAQRVLSRRLKGSSRWNKQRVKV | | | |
| | | SRIHEYISNTRKDYLDKISTEIIKNHD | | | |
| | | VIGIEDLHVSNMLKNHKLAKAISEV | | | |
| | | SWSQFRSMLEYKAKWYGKQVIVVS | | | |
| | | KTFASSQLCSCCGYQNKDVKNLNL | | | |
| | | REWDCFFCRTHHDRDINASINLKNE | | | |
| | | AIRLLTARTAGLA | | | |
| DDE_Tnp_1 | WP_016085235.1 | MSLSIQEEFHLFAQELQQYLSPHILQ | 1459 | Transposase | Bacillus cereus |
| | | QLAQETGFVKRKSKYGARDLAALCI | | DDE domain | BAG2O-1 |
| | | WISQHVASDSLTRLCSQLYANTATLM | | | |
| | | SPEGLNQRFNRCAVLFLQRVFSLLIK | | | |
| | | SKLNDFSQISNQYTSYFQRIRILDATI | | | |
| | | FQVPNHLAPIYPGSGGCAQTAGIKIQ | | | |
| | | LEYDLHSGKFLNFQMEPGKNNDKT | | | |
| | | FGTDCLDTLRPGDLCIRDLGYFSLK | | | |
| | | DLDQMDQRGVFYVSRLKLNNRVYV | | | |
| | | KNDYPEFFRDGTVKKQSLYVLLNLE | | | |
| | | DIMHQIKPGDTYENPKFFRSLEDKL | | | |
| | | AKAQRVLSRRLKGSSRWNKQRVKV | | | |
| | | SRIHEYISNTRKDYLDKISTEIIKNHD | | | |
| | | VIGIEDLHVSNMLKNHKLAKAISEV | | | |
| | | SWSQFRSMLEYKAKWYGKQVIVVS | | | |
| | | KTFASSQLCSCCGYQNKDVKNLNL | | | |
| | | REWDCPSCCTHHDRDINASINLKNE | | | |
| | | AIRLLTARTAGLA | | | |
| DDE_Tnp_1 | WP_016084599.1 | MSLSIQEEFHLFAQELQQYLSPHILQ | 1460 | Transposase | Bacillus cereus |
| | | QLAQETGFVKRKSKYGARDLAALCI | | DDE domain | BAG1X2-2 |
| | | WISQHVASDSLTRLCSQLYANTATLM | | | |
| | | SPEGLNQRFNRCAVLFLQRVFSLLIK | | | |
| | | SKLNDFSQISNQYTSYFQRIRILDATI | | | |
| | | FQVPNHLAPIYPGSGGCAQTAGIKIQ | | | |
| | | LEYDLHSGKFLNFQMEPGKNNDKT | | | |
| | | FGTDCLDTLRPGDLCIRDLGYFSLK | | | |
| | | DLDQMDQRGVFYVSRLKLNNRVYV | | | |
| | | KNDYPEFFRDGTVKKQSLYVLLNLE | | | |
| | | DIMHQIKPGDTYENPKFFRSLEDKL | | | |
| | | AKAQRVLSRRLKGSSRWNKQRVKV | | | |
| | | SRIHEYISNTRKDYLDKISTEIIKNHD | | | |
| | | VIGIEDLHVSNMLKNHKLAKAISEV | | | |
| | | SWSQFRAMLEYKAKWYGKQVIVVS | | | |
| | | KTFASSQLCSCCGYQNKDVKNLNL | | | |
| | | RKWDCPSCQTNHDRDINASINLKNE | | | |
| | | AIRLLTARTAGLA | | | |
| DDE_Tnp_1 | MBB5866658.1 | MRWRVRVGAPWRDVPPCYGTWQA | 1461 | Transposase | Allocatelliglobosispora |
| | | VYGLFRRKQRAGVWLRLVAGLQRR | | DDE domain | scoriae |
| | | ADALGLIGWDVSVDATTVRAHQHA | | | |
| | | AGARRNGDAQAEPPVGEPADHAFG | | | |
| | | RSRGGWTTKLHLACEQGRKPLSML | | | |
| | | LTAGHRGDSPQFAAVLAGIRVVGRV | | | |
| | | VGGVWHGVVARFTAFRFTVDPTPG | | | |
| | | QEVLLRRYAGASRFGYNQCLRLVK | | | |
| | | DALDAKVRGGVMKVPWTGFDLVN | | | |
| | | AFNAWKRSGDAGRVMVAAGDGTV | | | |
| | | SIEATGLVWRAEVSQQVFEEAAVDL | | | |
| | | GRALAAYTGSKAGARAGRRVGFPR | | | |
| | | FKSKKRTRLGFRVRCKTSRAGKADV | | | |
| | | RVGDNVARSVTLPGIGVLVVREDTR | | | |
| | | QLRRMLSKGRAKVLSATVGYRAGR | | | |
| | | WFVSLTCEAADLHQARQHPQPDPA | | | |
| | | DAATGTTGCGWVGVDRGLSAFVVA | | | |
| | | ARADGTPVLRVDDPPRPSRAGMGW | | | |
| | | QRRLARSVSRKQLGSANRRDAAAR | | | |
| | | LANHHAYVRTVRQRFLHHVSNQLV | | | |
| | | KTHDRLALETLNITGMLRTHRLAAA | | | |
| | | IADAAWAELARQVTYKQAWHGGRV | | | |
| | | VLVDRWYPSTKTCSACRTITPAMPL | | | |
| | | GQRVSTCGTCGYRADRDHNAAVNL | | | |
| | | AVWAEQHHARPGTSTQGARSPTPAE | | | |
| | | GKALARAPARVKPAPTTWEPHPPPP | | | |
| | | E | | | |
| DDE_Tnp_1 | MBP2579587.1 | MDENTTTVVVRETLDPTADQRAILQ | 1462 | Transposase | Streptomyces sp. |
| | | RYADASRCSFNYALGLKHGAQQLW | | DDE domain | PvR006 |
| | | AHGRDQLVAQGQTPAEAARNAPKIE | | | |
| | | VPSQFAVQKIFLAQRDQPLPGPQLPG | | | |
| | | QEPRLLFPWWKGVNAIVCQQAFRD | | | |
| | | ADAAFSNWKSAGRRKGVPVGYPRF | | | |
| | | KRRGRRRDSFRMFAVRLVEQDLRH | | | |
| | | VRIGGGGGQPAFSVRLHRPARRLAR | | | |
| | | LLARGGVAKSVTISREGHRWVAAFN | | | |
| | | VRVPVGPVPRPSRRQREAGAVGVDL | | | |
| | | GVKVFVATSDPVVINDHKIQLFENA | | | |
| | | RHLENTRRQLRKWQRRMARRHVR | | | |
| | | GLRSHEQSQGWRDARDQVARLHAL | | | |
| | | VAARRASSQHLVTKRLVTQYAHVAL | | | |
| | | EDLRVKSMTASARGAVESPGRNVRA | | | |
| | | KAGLNRAILDVGFGEIRRQIEYKAVL | | | |
| | | NGTRVTVVDPAYTSQTCNRCGHVD | | | |
| | | AKSRRTAISSPAPTAATPLTPTIPSRSC | | | |
| | | WRRSSPSSPPTSPVSRSRSTRRCTAY | | | |
| | | TSRICVTTLQTSSSPGSPPWTPSGSRS | | | |
| | | PTPTKAGDSAAPFVAFGRLRTFTPKG | | | |
| | | CCRRLSSGHFPCMGGVLRAEPVWV | | | |
| | | ETFTGLRMDRFVKLVKVVRERGGN | | | |
| | | GPGGGRPWCLPLPDRVLLVAVYYRT | | | |
| | | NLTMRQLAPLFGISPATVCRVIQRLR | | | |
| | | PLLALERAPQPVVDTERLWIVDGTLI | | | |
| | | PVRDRKVGASSRNYRFSANVQVIID | | | |
| | | ADTRLVIAAARPAPGNKADAHVWR | | | |
| | | GSDLPALAAGTTVIADGAYLGTGLI | | | |
| | | VPHRKRAGRPLLRGQEEDNAEHRR | | | |
| | | VRARVEHTFARMKNWKILRDCRQK | | | |
| | | GDGLHHAIQAAATMHNLAMTR | | | |
| DDE_Tnp_1_7 | KAG9067727.1 | MDSSLEFLGSFDVSDSQGTVVASAE | 1463 | Transposase | Linnemannia |
| | | EVDEAQEEEEELLFSVRCRTRPTPGT | | IS4 | hyalina |
| | | DSSGDEEEEKAGDEGVIDASAPPTK | | | |
| | | KIFSDPVLTDLADNTNAYAASKGAG | | | |
| | | TGEGSRQWVKTTPDELRTFLGIIVY | | | |
| | | MGVFRQNSVSEYWSTFPECPQHNIT | | | |
| | | TFMSLVRFEQLKRFFHVSNPNEPEQ | | | |
| | | HWFSKVEPQASSGPERAADVDGAG | | | |
| | | MSTHTTTSTRQASSSPKRAADVDDA | | | |
| | | DTADTASSNKRLRPLQISSGSSTLVE | | | |
| | | DMQRLLATQPSQIRFDESGALTTICA | | | |
| | | QEKDFRSVGAAIEKILSSRLSKDITM | | | |
| | | LHMDGLRSMEKEWAHGKRDQALS | | | |
| | | KQLETLERDYTEGKLHNKRQLYKR | | | |
| | | LKASYRAPPEALRAVSEVLRQSGWT | | | |
| | | ICQCLNQSDTCIARTVNNAAVPGDIR | | | |
| | | VITKDSDLMAFESIMSVTMPVKNTW | | | |
| | | TTFHKDELLNEHGLPTPVHLTLAAL | | | |
| | | VSNNDYTNGVFSYGLTSNVDTIRQF | | | |
| | | KMTGLDGTVGQDRVEVVRIYVRRY | | | |
| | | LDIIHQKARTIKDSATQSARRRLRCN | | | |
| | | PNPTVKAHDKDLRRIETADRQLRVD | | | |
| | | VTEFGHALKTFGAATDAEATPPPLPT | | | |
| | | APKAGSAPYPQATSAGSPSIRQTHGP | | | |
| | | AEHPPSHKQKIKKQRRHGSRALQRR | | | |
| | | RQKWRRSRFRSRTDVQDRYVPDTV | | | |
| | | FLEKASPVDVVELSGLKPSTPRPSKP | | | |
| | | KEQSPRIDQVPAPTAKKKKKKLLGE | | | |
| | | PKGIAGPKALKRAFQSVFATVTLTTG | | | |
| | | SLQGCLGRSTNLSKAEVAQLTQHVS | | | |
| | | SAVSTVNSAKHIVYKLIEMRILQPLIE | | | |
| | | TGLNQAEDGPDESFLEKILDSDWAE | | | |
| | | RFVQNLLSFVLRNSIVPQGRPPASDK | | | |
| | | SKDAVAEAISTFNEFKKTLCPGFKAL | | | |
| | | NSTDLALSNIIAELAPKICLDQKLHY | | | |
| | | RRIPETLRTKLSKLSIDCDGLPEIDQD | | | |
| | | GTDAGGDAGAADVNEGVDDDDAL | | | |
| | | KRSKKIIFKPGHIQLCWRYFLLLPSS | | | |
| | | KRPRFCTQAKTSDSFIDINEEALVAL | | | |
| | | LWGEKAVQLDNVWEDTRYTHNWA | | | |
| | | AAKQRSSYGEVIKELFIGDRDVIKEA | | | |
| | | RNKQQTTYGKRTTTMAEREEAHPHI | | | |
| | | YGQLELARYLINKVNFFRERHNASL | | | |
| | | TAPTPPLPSSTPSSSTTASSHPTSAAAI | | | |
| | | EKLYPTRQHLIDAFGDDLDSVIVVGI | | | |
| | | DPGEVVSGAFCLTLPGGKVINLLIKR | | | |
| | | ASLYQPTLAFRDWEQHWKRRHPTA | | | |
| | | GPGDVVDSSLWTRITDLDKLTTLPS | | | |
| | | VHDLENSLPSTNYDTSLDALTAAHK | | | |
| | | KYYEQEPLIHGIYASREWKVAVHEH | | | |
| | | RMAKMSELDLAVAGVLRMVDEACE | | | |
| | | GVPSAALGYKAALVDEYLTSTMCPT | | | |
| | | CVVENRATRLAKPSMRTCACVECTR | | | |
| | | WIHRDGVGAHNIALIGEQYLKSLGR | | | |
| | | PEPLARPPKQT | | | |
| DDE_Tnp_1_7 | KAG9062473.1 | MDSSLEFLGSFDVSDSQGTVVASAE | 1464 | Transposase | Linnemannia |
| | | EVDEAQEEEELLFSVRCRTRPTPGTD | | IS4 | hyalina |
| | | SSGDEEEEKAGDEGVIDASAPPTKKI | | | |
| | | FSDPVLTDLADNTNAYAASKGAGTG | | | |
| | | EGSRQWVKTTPDELRTFLGIIVYMG | | | |
| | | VFRQNSVSEYWSTFPECPQHNITTF | | | |
| | | MSLVRFEQLKRFFHVSNPNEPEQHW | | | |
| | | FSKVEPQASSGPERAADVDGAGMS | | | |
| | | THTTTSTRQASSSPKRAADVDDADT | | | |
| | | ADTASSNKRLRPLQISSGSSTLVEDM | | | |
| | | QRLLATQPSQIRFDESGALTTICAQE | | | |
| | | KVIDFRSVGAAIEKILSSRLSKDITML | | | |
| | | HMDGLRSMEKEWAHGKRDQALSK | | | |
| | | QLETLERDYTEGKLHNKRQLYKRL | | | |
| | | KASYRAPPEALRAVSERQCIARTVN | | | |
| | | NAAVPGDIRVITKDFDLMAFESIMSV | | | |
| | | TMPVKNTWTTFHKDELLNEHGLPT | | | |
| | | PVHLTLAALVSNNDYTNGVFSYGLT | | | |
| | | SNVDTVRQFKMTGLDGTVGQDRVE | | | |
| | | VVRIYVRRYLDIIHQKARTIKDSATQ | | | |
| | | SARRRLRCNPNPTVKAHDKDLRRIE | | | |
| | | TADRQLRVDVTEFGHALKTFGAATD | | | |
| | | AEATPPPLPTAPKAGSAPYPQATSAG | | | |
| | | SPSIRQTHGPAEHPPSHKQKIKKQRR | | | |
| | | HGSHALQRRRQKWRRSRFRSRTDV | | | |
| | | QDRYVPDTVFLEKASPVDVVELSGL | | | |
| | | KPSTPRPSKPKEQPPRIDQVPAPAAK | | | |
| | | KKKKKLLGEPKGIAGPKALKRAFQS | | | |
| | | VFATVTLTTGSLQGCLGRSTNLSKAE | | | |
| | | VAQLTQHVSSAVSTVNSAKHIVYKLI | | | |
| | | EMRILQPLIETGLNQAEDGPDESFLE | | | |
| | | KILDSDWAERFVQNLLSFVLRNSIVP | | | |
| | | QGRPPASDKSKDAVAEAISTFNEFKK | | | |
| | | TLCPGFKALNSTDLALSNIIAELAPKI | | | |
| | | CLDQKLHYRRIPETLRTKLSKLSIDC | | | |
| | | DGLPEIDQDGTDAGGDAGAADVNE | | | |
| | | GVDDDDALKRSKKIIFKPGHIQLCW | | | |
| | | RYFLLLPSSKRPRFCTQAKMSDSFID | | | |
| | | INEEALVALLWGEKAVQLDNVWED | | | |
| | | TRYTHNWAAAKQRSSYGEVIKELFI | | | |
| | | GDRDVIKEARNKQQTTYGKRTTTM | | | |
| | | AEQRHNASLTAPTPPLPSSTPSSSTTS | | | |
| | | TPPPLPTPRQQPRSYRYALNNYIRTD | | | |
| | | GHQLQILAYDLTKPRQSPNYSEFLSR | | | |
| | | IEKLYPTRQHLIDAFGDDLDSVIVVG | | | |
| | | IDPGEVVSGAFCLTLPGGKVINLLIK | | | |
| | | RASLYQPTLAFRDWEQHWKRRHPT | | | |
| | | AGPGDVVDSSLWTRITDLDKLTTLP | | | |
| | | SVHDLENSLPSTNYDTSLDALTAAH | | | |
| | | KKYYEQEPLIHGIYASREWKVAVHE | | | |
| | | HRMAKMSELDLAVAGVLRMVDEA | | | |
| | | CEGVPSAALGYKAALVDEYLTSTM | | | |
| | | CPTCVVENRATRLAKPSMRTCACVE | | | |
| | | CTRWIHRDGVGAHNIALIGEQYLKS | | | |
| | | LGRPEPLARPPKQT | | | |
| DDE_Tnp_1_7 | KAG9072475.1 | MDSSLEFLGSFDVSDSQGTVVASAE | 1465 | Transposase | Linnemannia |
| | | EVDEAQEEEELLFSVRCRTRPTPGTD | | IS4 | hyalina |
| | | SSGDEEEEKAGDEGVIDASAPPTKKI | | | |
| | | FSDPVLTDLADNTNAYAASKGAGTG | | | |
| | | EGSRQWVKTTPDELRTFLGIIVYMG | | | |
| | | VFRQNSVSEYWSTFPECPQHNITTF | | | |
| | | MSLVRFEQLKRFFHVSNPNEPEQHW | | | |
| | | FSKVEPQASSGPERAADVDGAGMS | | | |
| | | THTTTSTRQASSSPKRAADVDDADT | | | |
| | | ADTASSNKRLRPLQISSGSSTLVEDM | | | |
| | | QRLLATQPSQIRFDESGALTTICAQE | | | |
| | | KVIDFRSVGAAIEKILSSRLSKDITML | | | |
| | | HMDGLRSMEKEWAHGKRDQALSK | | | |
| | | QLETLERDYTEGKLHNKRQLYKRL | | | |
| | | KASYRAPPEALRAVSEVLRQSGWTI | | | |
| | | CQCLNQSDTCIARTVNNAAVPGDIR | | | |
| | | VITKDSDLMAFESIMSVTMPVKNTW | | | |
| | | TTFHKDELLNEHGLPTPVHLTLAAL | | | |
| | | VSNNDYTNGVFSYGLTSNVDTIRQF | | | |
| | | KMTGLDGTVGQDRVEVVRIYVRRY | | | |
| | | LDIIHQKARTIKDSATQSARRRLRCN | | | |
| | | PNPTVKAHDKDLRRIETADRQLRVD | | | |
| | | VTEFGHALKTFGAATDAEATPPPLPT | | | |
| | | APKAGSAPYPQATSAGSPSIRQTHGP | | | |
| | | AEHPPSHKQKIKKQRRHGSRALQRR | | | |
| | | RQKWRRSRFRSRTDVQDRYVPNTV | | | |
| | | FLEKASPVDVVELSGLKPSTPRPSKP | | | |
| | | KEQSPRINQVPAPAAKKKKKKLLGE | | | |
| | | PKGIAGPKALKRAFQSVFATVTLTTG | | | |
| | | SLQGCLGRSTNLSKAEVAQLTQHVS | | | |
| | | SAVSTVNSAKHIVYKLIEMRILQPLIE | | | |
| | | TGLNQAEDGPDESFLEKILDSDWAE | | | |
| | | RFVQNLLSFVLRNSIVPQGRPPASDK | | | |
| | | SKDAVAEAISTFNEFKKTLCPGFKAL | | | |
| | | NSTDLALSNIIAELAPKICLDQKLHY | | | |
| | | RRIPETLRTKLSKLSIDCDGLPEIDQD | | | |
| | | DTDAGGDAGAADVNEGVDDDDAL | | | |
| | | KRSKKIIFKPGHIQLCWRYFLLLSSS | | | |
| | | KRPRFCTQAKMSDSFIDINEEALVAL | | | |
| | | LWGEKAAQLDNVWEDTRYTHNWA | | | |
| | | AAKQRSSYGEVIKELFIGDRDVIKEA | | | |
| | | RNKQQTTYGKRTTTMAEREEAHPHI | | | |
| | | YGQLELARYLTNKVNFFRERHNASL | | | |
| | | TAPTPPLPSSTSSSSTTSTPPPLPTPRQ | | | |
| | | QPRSYRYALNNYIRTDGHQLQILAY | | | |
| | | DLTKPRQSPNYSEFLSRIEKLYPTRQ | | | |
| | | HLIDAFGDDLDSVIVVGIDPGEVVSG | | | |
| | | AFCLTLPGGKVINLLIKRASLYQPTL | | | |
| | | AFRDWEQHWKRRHPTAGPGDVVD | | | |
| | | SSLWTRITDLDKLTTLPEFSPSTNYD | | | |
| | | TSLDALTAAHKKYYEQEPLIHGIYAS | | | |
| | | REWKVAVHEHRMAKMSELDLAVAG | | | |
| | | VLRMVDEACEGVPSAALGYKAALV | | | |
| | | DEYLTSTMCPTCVVENRATRLAKPS | | | |
| | | MRTCACVECTRWIHRDGVGAHNIA | | | |
| | | LIGEQYLKSLGRPEPLARPPNKPNLY | | | |
| | | R | | | |
| DDE_Tnp_1_7 | KAG9064049.1 | MDSSLEFLGSFDVSDSQGTVVASAE | 1466 | Transposase | Linnemannia |
| | | EVDEAQEEEELLFSVRCRTRPTPGTD | | IS4 | hyalina |
| | | SSGDEEEEKAGDEGVIDASAPPTKKI | | | |
| | | FSDPVLTDLADNTNAYAASKGAGTG | | | |
| | | EGSRQWVKTTPDELRTFLGIIVYMG | | | |
| | | VFRQNSVSEYWSTFPECPQHNITTF | | | |
| | | MSLVRFEQLKRFFHVSNPNEPEQHW | | | |
| | | FSKVEPQASSGPERAADVDGAGMS | | | |
| | | THTTTSTRQASSSPKRAADVDDADT | | | |
| | | ADTASSNKRLRPLQISSGSSTLVEDM | | | |
| | | QRLLATQPSQIRFDESGVLTTICAQE | | | |
| | | KVIDFRSVGAAIEKILSSRLSKDITML | | | |
| | | HMDGLRSMEKEWAHGKRDQALSK | | | |
| | | QLETLERDYTEGKLHNKRQLYKRL | | | |
| | | KASYRAPPEALRAVSEVLRQSGWTI | | | |
| | | CQCLNQSDTCIARTVNNAAVPGDIR | | | |
| | | VITKDSDLMAFESIMSVTMPVKNTW | | | |
| | | TTFHKDELLNEHGLPTPVHLTLAAL | | | |
| | | VSNNDYTNGVFSYGLTSNVDTIRQF | | | |
| | | KMTGLDGTVGQDRVEVVRIYVRRY | | | |
| | | LDIIHQKARTIKDSATQSARRRLRCN | | | |
| | | PNPTVKAHDKDLRRIETADRQLRVD | | | |
| | | VTEFGHALKTFGAATDAEATPPPLPT | | | |
| | | APKAGSAPYPQATSAGSPSIRQTHGP | | | |
| | | AEHPPSHKQKIKKQRRHGSRALQRR | | | |
| | | RQKWRRSRFRSRTDVQDRYVPDTV | | | |
| | | FLEKASPVDVVELSGLKPSTPRPSKP | | | |
| | | KEQSPRIDQVPAPAAKKKKKKLLGE | | | |
| | | PKGIAGPKALKRAFQSVFATVTLTTG | | | |
| | | SLQGCLGRSTNLSKAEVAQLTQHVS | | | |
| | | SAVSTVNSAKHIVYKLIEMRILQPLIE | | | |
| | | TGLNQAEDGPDESFLEKILDSDWAE | | | |
| | | RFVQNLLSFVLRNSIVPQGRPPASDK | | | |
| | | SKDAVAEAISTFNEFKKTLCPGFKAL | | | |
| | | NSTDLALSNIIAELAPKICLDQKLHY | | | |
| | | RRIPETLRTKLSKLSIDCDGLPEIDQD | | | |
| | | GTDAGGDAGAADVNEGVDDDDAL | | | |
| | | KRSKKIIFKPGHIQLCWRYFLLLPSS | | | |
| | | KRPRFCTQAKMSDSFIDINEEALVAL | | | |
| | | LWGEKAVQLDNVWEDTRYTHNWA | | | |
| | | AAKQRSSYGEVIKELFIGDRDVIKEA | | | |
| | | RNKQQTTYGKRTTTMAEREEAHPHI | | | |
| | | YGQLELARYLTNKVNFFRERHNASL | | | |
| | | TAPTPPLPSSTPSSSTTSTPPPLPTPRQ | | | |
| | | QPRSYRYALNNYIRTDGHQLQILAY | | | |
| | | DLTKPRQSPNYSEFLSRIEKLYPTRQ | | | |
| | | HLIDAFGDDLDSVIVVGIDPGEVVSG | | | |
| | | AFCLTLPGGKVINLLIKRASLYQPTL | | | |
| | | AFRDWEQHWKRRHPTAGPGDVVD | | | |
| | | SSLWTRITDLDKLTTLPSVHDLENSL | | | |
| | | PSTNYDTSLDALTAAHKKYYEQEPL | | | |
| | | IHGIYASREWKVAVHEHRMAKMSEL | | | |
| | | DLAVAGVLRMVDEACEGVPSAALG | | | |
| | | YKAALVDEYLTSTMCPTCVVENRAT | | | |
| | | RLAKPSMRTCACVECTRWIHRDGV | | | |
| | | GAHNIALIGEQYLKSLGRPEPLARPP | | | |
| | | KQT | | | |
| DDE_Tnp_1_7 | KAG9061854.1 | MDSSLEFLGSFDVSDSQGTIVASAEE | 1467 | Transposase | Linnemannia |
| | | VDEAQEEELLFSVRCRTRPTPGTDSS | | IS4 | hyalina |
| | | GDEEEEEKKAGDEGVIGAPTPPTKK | | | |
| | | KAAKKAAKKPVRETRELPPVLDFD | | | |
| | | NIFRHYKDGHPAQSNLPRALRLDSE | | | |
| | | LSPLTIFTLFFSDPVLTDLADNTNAYA | | | |
| | | ASKGAGTGEGSRQWVKTTPDELRT | | | |
| | | FLGIIVYMGVFRQNSVSEYWSTFPE | | | |
| | | CPQHNITTFMSLVRFEQLKRFFHVSN | | | |
| | | PNEPEQHWFSKVEPQASSGPERAAD | | | |
| | | VDGAGMSTHTTTSTRQASSSPKRAA | | | |
| | | DVDDVDTADTASSNKRLRPLQISSG | | | |
| | | SSTLVEDMQRLLATQPSQIRFDESGA | | | |
| | | LTTICAQEKVIDFRSVGAAIEKILSSR | | | |
| | | LSKDITMLHMDGLRSMEKEWAHGK | | | |
| | | RDQALSKQLETLERDYTEGKLHNK | | | |
| | | RQLYKRLKASYRAPPEALRAVSEVL | | | |
| | | RQSGWTICQCLNQSDTCIARTVNNA | | | |
| | | AVPGDIRVITKDSDLMAFESIMSVTM | | | |
| | | PVKNTWTTFHKDELLNEHGLPTPVH | | | |
| | | LTLAALVSNNDYTNGVFSYGLTSNV | | | |
| | | DTIRQFKMTGLDGTVGQDRVEVVRI | | | |
| | | YVRRYLDIIHQKARTIKDSATQSARR | | | |
| | | RLRCNPNPTVKAHDKDLRRIETADR | | | |
| | | QLRVDVTEFGHALKTFGAATDAEAT | | | |
| | | PPPLPTAPKAGSAPYPQATSAGSPSIR | | | |
| | | QTHGPAEHPPSHKQKIKKQRRHGSR | | | |
| | | ALQRRRQKWRRSRFRSRTDVQDRY | | | |
| | | VPDTVFLEKASPVDVVELSGLKPSIP | | | |
| | | RPSKPKEQSPRIDQVPAPAAKKKKK | | | |
| | | KLLGEPKGIAGPKALKRAFQSVFAT | | | |
| | | VTLTTGSLQGCLGRSTNLSKAEVAQ | | | |
| | | LTQHVSSAVSTVNSAKHIVYKLIEM | | | |
| | | RILQPLIETGLNQAEDGPDESFLEKIL | | | |
| | | DSDWAERFVQNLLSFVLRNSIVPQG | | | |
| | | RPPASDKSKDAVAEAISTFNEFKKTL | | | |
| | | CPGFKALNSTDLALSNIIAELAPKICL | | | |
| | | DQKLHYRRIPETLRTKLSIDCDGLPE | | | |
| | | IDQDDTDAGGDAGAADVNEGVDD | | | |
| | | DDALKRSKKIIFKPGHIQLCWRYFLL | | | |
| | | LPSSKRPRFCTQAKMSDSFIDINEEA | | | |
| | | LVALLWGEKAVQLDNVWEDTRYTH | | | |
| | | NWAAAKQRSSYGEVIKELFIGDRDV | | | |
| | | IKEARNKQQTTYGKRTTTMAEREEA | | | |
| | | HPHIYGQLELARYLTNKVNFFRERH | | | |
| | | NASLTAPTPPLPSSTPSSSTTSTPPPLP | | | |
| | | TPRQQPRSYRYALNNYIRTDGHQLQI | | | |
| | | LAYDLTKPRQSPNYSEFLSRIEKLYP | | | |
| | | TRQHLIDAFGDDLDSVIVVGIDPGEV | | | |
| | | VSGAFCLTLPGGKVINLLIKRASLYQ | | | |
| | | PTLAFRDWEQHWKRRHPTAGPGDV | | | |
| | | VDSSLWTRITDLDKLTTLPSVHDLEN | | | |
| | | SLPSTNYDTSLDALTAAHKKYYEQE | | | |
| | | PLIHGIYASREWKVAAHEHRMAKM | | | |
| | | SELDLAVAGVLRMVDEACEGVPVH | | | |
| | | QRKSAALGYKAALVDEYLTSTMCP | | | |
| | | TCVVENRATRLAKPSMRTCACVECT | | | |
| | | RWIHRDGVGAHNIALIGEQYLKSLG | | | |
| | | RPEPLARPPKQT | | | |
| DDE_Tnp_1_7 | KAG9064695.1 | MDSSLEFLGSFDVSDSQGTVVASAE | 1468 | Transposase | Linnemannia |
| | | EVDEAQEEEELLFSVRCRTRPTPGTD | | IS4 | hyalina |
| | | SSGDEEEEKAGDEGVIDASAPPTKKI | | | |
| | | FSDPVLTDLADNTNAYAASKGAGTG | | | |
| | | EGSRQWVKTTPDELRTFLGIIVYMG | | | |
| | | VFRQNSVSEYWSTFPECPQHNITTF | | | |
| | | MSLVRFEQLKRFFHVSNPNEPEQHW | | | |
| | | FSKVEPQASSGPERAADVDGAGMS | | | |
| | | THTTTSTRQASSSPKRAADVDDADT | | | |
| | | ADTASSNKRLRPLQISSGSSTLVEDM | | | |
| | | QRLLATQPSQIRFDESGALTTICAQE | | | |
| | | KVIDFRSVGAAIEKILSSRLSKDITML | | | |
| | | HMDGLRSMEKEWAHGKRDQALSK | | | |
| | | QLETLERDYTEGKLHNKRQLYKRL | | | |
| | | KASYRAPPEALRAVSEVLRQSGWTI | | | |
| | | CQCLNQSDTCIARTVNNAAVPGDIR | | | |
| | | VITKDSDLMAFESIMSVTMPVKNTW | | | |
| | | TTFHKDELLNEHGLPTPVHLTLAAL | | | |
| | | VSNNDYTNGVFSYGLTSNVDTIRQF | | | |
| | | KMTGLDGTVGQDRVEVVRIYVRRY | | | |
| | | LDIIHQKARTIKDSATQSARRRLRCN | | | |
| | | PNPTVKAHDKDLRRIETADRQLRVD | | | |
| | | VTEFGHALKTFVLCEETTLDNNRISP | | | |
| | | AADTHTRLMAIIKETEFNKAYNDW | | | |
| | | RRFSASRTGSLPPGLLVDPASQGAAT | | | |
| | | DAEATPPPLPTAPKAGSAPYPQATSA | | | |
| | | GSPSIRQTHGPAEHPPSHKQKIKKQR | | | |
| | | RHGSRALQRRRQKWRRSRFRSRTD | | | |
| | | VQDRYVPNTVFLEKASPVDVVELSG | | | |
| | | LKPSTPRPSKPKEQSPRINQVPAPAA | | | |
| | | KKKKKKLLGEPKGIAGPKALKRAF | | | |
| | | QSVFATVTLTTGSLQGCLGRSTNLSK | | | |
| | | AEVAQLTQHVSSAVSTVNSAKHIVY | | | |
| | | KLIEMRILQPLIETGLNQAEDGPDES | | | |
| | | FLEKILDSDWAERFVQNLLSFVLRNS | | | |
| | | IVPQGRPPASDKSKDAVAEAISTFNE | | | |
| | | FKKTLCPGFKALNSTDLALSNIIAEL | | | |
| | | APKICLDQKLHYRRIPETLRTKLSID | | | |
| | | CDGLPEIDQDDTDAGGDAGAADVN | | | |
| | | EGVDDDDALKRSKKIIFKPGHIQLC | | | |
| | | WRYFLLLSSSKRPRFCTQAKMSDSFI | | | |
| | | DINEEALVALLWGEKAAQLDNVWE | | | |
| | | DTRYTHNWAAAKQRSSYGEVIKELF | | | |
| | | IGDRDVIKEARNKQQTTYGKRTTTM | | | |
| | | AEREEAHPHIYGQLELARYLTNKVN | | | |
| | | FFRERHNASFTAPTPPLPSSTSSSSTT | | | |
| | | STPPPLPTPRQQPRSYRYALNNYIRT | | | |
| | | DGHQLQILAYDLTKPRQSPNYSEFLS | | | |
| | | RIEKLYPTRQHLIDAFGDDLDSVIVV | | | |
| | | GIDPGEVVSGAFCLTLPGGKVINLLI | | | |
| | | KRASLYQPTLAFRDWEQHWKRRHP | | | |
| | | TAGPGDVVDSSLWTRITDLDKLTTL | | | |
| | | PSVHDLENSLPSTNYDTSLDALTAA | | | |
| | | HKKYYEQEPLIHGIYASREWKVAVH | | | |
| | | EHRMAKMSELDLAVAGVLRMVDE | | | |
| | | ACEGVPVHQRKVFFALGNGTFRSGF | | | |
| | | NLSSVHITFLRRLLQQSAALGYKAA | | | |
| | | LVDEYLTSTMCPTCVVENRATRLAK | | | |
| | | PSMRTCACVECTRWIHRDGVGAHNI | | | |
| | | ALIGEQYLKSLGRPEPLARPPKQT | | | |
| DDE_Tnp_1_7 | KAG9068923.1 | MDSSLEFLGSFDVSDSQGTVVASAE | 1469 | Transposase | Linnemannia |
| | | EVDEAQEEEELLFSVRCRTRPTPGTD | | IS4 | hyalina |
| | | SSGDEEEEKAGDEGVIDASAPPTKK | | | |
| | | MKAAKKAAKKPVRETRELPPVPDF | | | |
| | | DNIFRHYKDGHPAQSNLPRALRLDS | | | |
| | | ELSPLTIFTLFFSDPVLTDLADNTNAY | | | |
| | | AASKGAGTGEGSCQWVKTTPYELR | | | |
| | | TFLGIIVYMGVFRQNSVSEYWSTFPE | | | |
| | | CPQHNITTFMSLVRFEQLKRFFHVSN | | | |
| | | PNEPEQHWFSKVEPQTSSGPERAAD | | | |
| | | VDGAGMSTHTTTSTRQASSSPKRAA | | | |
| | | DVDDADTADTASSNKRLRPLQISSG | | | |
| | | SSTLVEDMQRLLSTQPSQIRFDESGA | | | |
| | | LTTICAQEKVIDFRSVGAAIEKILSSR | | | |
| | | LSKDITMLHMDGLRSMEKEWAHGK | | | |
| | | RDQALSKQLETLERDYTEGKLHNK | | | |
| | | RQLYKRLKASYRAPPEALRAVSEVL | | | |
| | | RQSGWTICQCLNQSDTCIARTVNNA | | | |
| | | AVPGDIRVITKDSDLMAFESIMSVTM | | | |
| | | PVKNTWTTFHKDELLNEHGLPTPNN | | | |
| | | DYTNGVFSYGLTSNVDTIRQFKMTG | | | |
| | | LDGTVGQDRVEVVRIYVRRYLDIIH | | | |
| | | QKARTIKDSATQSARRRLRCNPNPT | | | |
| | | VKAHDKDLRRIETADRQLRVDVTEF | | | |
| | | GHALKTFVLCEETTLDNNRISPAAD | | | |
| | | THTRLMAIIKETEFNKAYNDWRRFS | | | |
| | | ASRRGSLPPGLLVDPASQGAATDAE | | | |
| | | ATPPPLPTAPKAGSAPYPQATSAGSP | | | |
| | | SIRQTHGPAEHPPSHKQKIKKQRRH | | | |
| | | GSRALQRRRQKWRRSRFRSRTDVQ | | | |
| | | DRYVPDTVFLEKASPVDVVELSGLK | | | |
| | | PSTPRPSKPKEQSPRIDQVPAPAAKK | | | |
| | | KKKKLLGEPKGIAGPKALKRAFQSV | | | |
| | | FATVTLTTGSLQGCLGRSTNLSKAEV | | | |
| | | AQLTQHVSSAVSTVNSAKHIVYKLIE | | | |
| | | MRILQPLIETGLNQAEDGPDESFLEK | | | |
| | | ILDSDWAERFVQNLLSFVLRNSIVPQ | | | |
| | | GRPPASDKSKDAVAEAISTFNEFKKT | | | |
| | | LCPGFKALNSTDLALSNIIAELAPKIC | | | |
| | | LDQKLHYRRIPETLRTKLSIDCDGLP | | | |
| | | EIDQDDTDADGDAGAADVNEGVDD | | | |
| | | DDALKRSKKIIFKPGHIQLCWRYFLL | | | |
| | | LPSSKRPRFCTQAKMSDSFIDINEEA | | | |
| | | LVALLWGEKAVQLDNVWEDTRYTH | | | |
| | | NWAAAKQRSSYGEVIKELFIGDRDV | | | |
| | | IKEARNKQQTTYGKRTTTMAEREEA | | | |
| | | HPHIYGQLELARYLTNKVNFFRERH | | | |
| | | NASLTAPTPPLPSSTSSSSTTSTPPPLP | | | |
| | | TPRQQPRSYRYALNNYIRTDGHQLQI | | | |
| | | LAYDLTKPRQSPKYSEFLSRIEKLYP | | | |
| | | TRQHLIDAFGDDLDSVIVVGIDPGEV | | | |
| | | VSGAFCLTLPGGKVINLLIKRASLYQ | | | |
| | | PTLAFRDWEQHWKRRHPTAGPGDV | | | |
| | | VDSSLWTRITDLDKLTTLPSVHDLEN | | | |
| | | SLPSTNYDTSLDALTAAHKKYYEQE | | | |
| | | PLIHGIYASREWKVAVHEHRMAKMS | | | |
| | | ELDLAVAGVLRMVDEACEGVPVHQ | | | |
| | | RKSAALGYKAALVDEYLTSTMCPTC | | | |
| | | VVENRATRLAKPSMRTCACVECTR | | | |
| | | WIHRDGVGAHNIALIGEQYLKSLGR | | | |
| | | PEPLARPPKPT | | | |
| DDE_Tnp_1_7 | KAG9069025.1 | MDSSLEFLGSFDVSDSQGTVVASAE | 1470 | Transposase | Linnemannia |
| | | EVDEAQEEEELFSVRCRTRPTPGTDS | | IS4 | hyalina |
| | | SGDEEEEEKAGDEGVIDATAPPTKK | | | |
| | | MKAAKKAAKKPVRETRELPPVLDF | | | |
| | | DNIFRHYKDGHPAQSNLPRALRLDS | | | |
| | | ELSPLTIFTLFFSDPVLTDLADNTNAY | | | |
| | | AASKGAGTGEGSRQWVKTTPDELR | | | |
| | | TFLGIIVYMGVFRQNSVSEYWSTFPE | | | |
| | | CPQHNITTFMSLVRFEQLKRFFHVSN | | | |
| | | PNEPEQHWFSKVEPQTSSGPERAAD | | | |
| | | VDGAGMSTHTTTSTRQASSSPKRAA | | | |
| | | DVDDADTADTASSNKRLRPLQISSG | | | |
| | | SSTLVEDMQRLLATQPSQIRFDESGA | | | |
| | | LTTICAQEKVIDFRSVGAAIEKILSSR | | | |
| | | LSKDITMLHMDGLRSMEKEWAHGK | | | |
| | | RDQALSKQLETLERDYTEGKLHNK | | | |
| | | RQLYKRLKASYRAPPEALRAVSEVL | | | |
| | | RQSGWTICQCLNQSDTCIARTVNNA | | | |
| | | AVPGDIRVITKDSDLMAFESIMSVTM | | | |
| | | PVKNTWTTFHKDELLNEHGLPTPVH | | | |
| | | LTLAALVSNNDYTNGVFSYGLTSNV | | | |
| | | DTIRQFKMTGLDGTVGQDRVEVVRI | | | |
| | | YVRRYLDIIHQKARTIKDSATQSARR | | | |
| | | RLRCNPNPTVKAHDKDLRRIETADR | | | |
| | | QLRVDVTEFGHALKTFVLCEETTLD | | | |
| | | NNRISPAADTHTRLMAIIKETEFNKA | | | |
| | | YNDWRRFSASRTGSLPPGLLVDPAS | | | |
| | | QGAATDAEATPPPLPTAPKAGSAPYP | | | |
| | | QATSAGSPSIRQTHGPAEHPPSHKQK | | | |
| | | IKKQRRHGSRALQRRRQKWRRSRF | | | |
| | | RSRTDVQDRYVPDTVFLEKASPVDV | | | |
| | | VELSGLKPSTPRPSKPKEQSPRIDQV | | | |
| | | PAPAAKKKKKKLLGEPKGIAGPKAL | | | |
| | | KRAFQSVFATVTLTTGSLQGCLGRST | | | |
| | | NLSKAEVAQLTQHVSSAVSTVNSAK | | | |
| | | HIVYKLIEMRILQPLIETGLNQAEDG | | | |
| | | PDESFLEKILDSDWAERFVQNLLSFV | | | |
| | | LRNSIVPQGRPPASDKSKDAVAEAIS | | | |
| | | TFNEFKKTLCPGFKALNSTDLALSNI | | | |
| | | IAELAPKICLDQKLHYRRIPETLRTK | | | |
| | | LSIDCDGLPEIDQDGTDAGGDAGAA | | | |
| | | DVNEGVDDDDALKRSKKIIFKPGHI | | | |
| | | QLCWRYFLLLPSSKRPRFCTQAKMS | | | |
| | | DSFIDINEEALVALLWGEKAVQLDN | | | |
| | | VWEDTRYTHNWAAAKQRSSYGDVI | | | |
| | | KELFIGDRDVIKEARNKQQTTYGKR | | | |
| | | TTTMAEREEAHPHIYGQLELARYLT | | | |
| | | NKVNFFRERHNASLTAPTPPLPSSTS | | | |
| | | SSSTTSTPPPLPTPRQQPRSYRYALN | | | |
| | | NYIRTDGHQLQILAYDLTKPRQSPNY | | | |
| | | SEFLSRIEKRYPTRQHLIDAFGDDLD | | | |
| | | SVIVVGIDPGEVVSGAFCLTLPGGKV | | | |
| | | INLLIKRASLYQPTLAFRDWEQHWK | | | |
| | | RRHPTAGPGDVVDSSLWTRITDLDK | | | |
| | | LTTLPSVHDLENSLPSTNYDTSLDAL | | | |
| | | TAAHKKYYEQEPLIHGIYASREWKV | | | |
| | | AVHEHRMAKMSELDLAVAGVLRMV | | | |
| | | DEACEGVPVHQRKSAALGYKAALV | | | |
| | | DEYLTSTMCPTCVVENRATRLAKPS | | | |
| | | MRTCACVECTRWIHRDGVGAHNIA | | | |
| | | LIGEQYLKSLGRPEPLARPPKQTKPL | | | |
| DDE_Tnp_1_7 | KAG9069522.1 | MVIRHLAVESDRRSLASLLRVNKYV | 1471 | Transposase | Linnemannia |
| | | CSATQPVMYADPFYLRPFSAFSMET | | IS4 | hyalina |
| | | PFSLIRLLSLIKLLLLSLPEGQVVTDL | | | |
| | | LRIAYLSTASASADDKTTEHQEQEAP | | | |
| | | PLISATFEVHSFSAGIFFTPPFPSYSAI | | | |
| | | LDYLENNGLAERYTARDVTSRLKH | | | |
| | | YEQPRIIRQGVKRDLRRDLTWALSIV | | | |
| | | GRLQVLSNVTLLLDSDLEPFLLFGQ | | | |
| | | QFEEGDQEVLELQQREREEHMEEMI | | | |
| | | LFVQEHRQRFPNTLNQGQCVTDTFT | | | |
| | | KEEWPKEVHDRLLQSLPPLYKPRFID | | | |
| | | NTNWVQFFARVEDVDLSAVKFIRTQ | | | |
| | | LIKPEEPVFDQVIEQVGPFLHRCRAL | | | |
| | | EDVQISSSGEEAFRWAVDERKQFER | | | |
| | | DIEDGRTTPRRPLVPLHQLHVSFDDH | | | |
| | | PSSGGLFDDIVFAFQETLEKIFIGICV | | | |
| | | SVEQNSTQSLEFSIGDNNNNNNNNN | | | |
| | | NNYYQSYWELPQLSRLAVATGHIFL | | | |
| | | HVHPKFLQRCTQVMHISLADMRRE | | | |
| | | YSLDQVNHWEPAKMPRLESLTLQG | | | |
| | | TAAISFHPDTPKYALELQKLHLQMV | | | |
| | | QNEDGMTCFIPPAEELDAIIEGESTID | | | |
| | | RGGSDNVDDGMTPSLLAPLARRPV | | | |
| | | WTWDWELPKLTDMTLTGEHAYRFQ | | | |
| | | FRMLDGTPNLINFSLDIVSTTALHQR | | | |
| | | TIHLKDLIKPGSQQQQQQQQKQQQQ | | | |
| | | QQQKQQQQQHQQTQDGEEIYEEQQ | | | |
| | | DLEYILVSTLKRLSLYGSWQMTTHIL | | | |
| | | QTLFSRVAPEISNLTMSSCLGHDFSE | | | |
| | | WVDATKQYLHGLVAAELNMDVSEE | | | |
| | | EEMADAGLVEAMMEEGILEFLGSFD | | | |
| | | VSDSQGTVVASAEEVDEAQEEEELL | | | |
| | | FSVRCRTRPTPGTDSSGDEEEEKAG | | | |
| | | DEGVIDASAPPTKKIFSDPVLTDLAD | | | |
| | | NTNAYAASKGAGTGEGSRQWVKTT | | | |
| | | PDELRTFLGIIVYMGVFRQNSVSEY | | | |
| | | WSTFPECPQHNITTFMSLVRFEQLKR | | | |
| | | FFHVSNPNEPEQHWFSKVEPQASSG | | | |
| | | PERAADVDGAGMSTHTTTSTRQASS | | | |
| | | SPKRAADVDDADTADTASSNKRLRP | | | |
| | | LQISSGSSTLVEDMQRLLATQSSQIR | | | |
| | | FDESGALTTICAQEKVIDFRSVGAAI | | | |
| | | EKILSSRLSKDITMLHMDGLRSMEK | | | |
| | | EWAHGKRDQALSKQLETLERDYTE | | | |
| | | GKLHNKRQLYKRLKASYRAPPEAL | | | |
| | | RAVSEVLRQSGWTICQCLNQSDTCI | | | |
| | | ARTVNNAAVPGDIRVITKDSDLMAF | | | |
| | | ESIMSVTMPVKNTWTTFHKDELLNE | | | |
| | | HGLPTPVHLTLAALVSNNDYTNGVF | | | |
| | | SYGLTSNVDTVRQFKMTGLDGTVG | | | |
| | | QDRVEVVRIYVRRYLDIIHQKARTIK | | | |
| | | DSATQSARRRLRCNPNPTVKAHDK | | | |
| | | DLRRIETADRQLRVDVTEFGHALKT | | | |
| | | FGAATDAEATPPPLPTAPKAGSAPYP | | | |
| | | QATSAGSPSIRQTHGPAEHPPSHKQK | | | |
| | | IKKQRRHGSRALQRRRQKWRRSRF | | | |
| | | RSRTDVQDRYVPDTVFLEKASPVDV | | | |
| | | VELSGLKPSTPRPSKPKEQSPRIDQV | | | |
| | | PAPAAKKKKKKLLGEPKGIAGPKAL | | | |
| | | KRAFQSVFATVTLTTGSLQGCLGRST | | | |
| | | NLSKAEVAQLTQHVSSAVSTVNSAK | | | |
| | | HIVYKLIEMRILQPLIETGLNQAEDG | | | |
| | | PDESFLEKILDSDWAERFVQNLLSFV | | | |
| | | LRNSIVPQGRPPASDKSKDAVAEAIS | | | |
| | | TFNEFKKTLCPGFKALNSTDLALSNI | | | |
| | | IAELAPKICLDQKLHYRRIPETLRTK | | | |
| | | LSKLSIDCDGLPEIDQDGTDAGGDA | | | |
| | | GAADVNEGVDDDDALKRSKKIIFKP | | | |
| | | GHIQLCWRYFLLLPSSKRPRFCTQA | | | |
| | | KMSDSFIDINEEALVALLWGEKAVQ | | | |
| | | LDNVWEDTRYTHNWAAAKQRSSY | | | |
| | | GEVIKELFIGDRDVIKEARNKQQTT | | | |
| | | YGKRTTTMAEREEAHPHIYGQLELA | | | |
| | | RYLTNKVNFFRERHNASLTAPTPPLP | | | |
| | | SSTPSSSTTSTPPPLPTPRQQPRSYRY | | | |
| | | ALNNYIRTDGHQLQILAYDLTKPRQ | | | |
| | | SPNYSEFLSRIEKLYPTRQHLIDAFG | | | |
| | | DDLDSVIVVGIDPGEVVSGAFCLTLP | | | |
| | | GGKVINLLIKRASLYQPTLAFRDWE | | | |
| | | QHWKRRHPTAGPGDVVDSSLWTRI | | | |
| | | TDLDKLTTLPSVHDLENSLPSTNYD | | | |
| | | TSLDALTAAHKKYYEQEPLIHGIYAS | | | |
| | | REWKVAVHEHRMAKMSELDLAVAG | | | |
| | | VLRMVDEACEGVPSAALGYKAALV | | | |
| | | DEYLTSTMCPTCVVENRATRLAKPS | | | |
| | | MRTCACVECTRWIHRDGVGAHNIA | | | |
| | | LIGEQYLKSLGRPEPLARPPKQT | | | |
| DDE_Tnp_4 | MBP2579587.1 | MDENTTTVVVRETLDPTADQRAILQ | 1472 | DDE | Streptomyces sp. |
| | | RYADASRCSFNYALGLKHGAQQLW | | superfamily | PvR006 |
| | | AHGRDQLVAQGQTPAEAARNAPKIE | | endonuclease | |
| | | VPSQFAVQKIFLAQRDQPLPGPQLPG | | | |
| | | QEPRLLFPWWKGVNAIVCQQAFRD | | | |
| | | ADAAFSNWKSAGRRKGVPVGYPRF | | | |
| | | KRRGRRRDSFRMFAVRLVEQDLRH | | | |
| | | VRIGGGGGQPAFSVRLHRPARRLAR | | | |
| | | LLARGGVAKSVTISREGHRWVAAFN | | | |
| | | VRVPVGPVPRPSRRQREAGAVGVDL | | | |
| | | GVKVFVATSDPVVINDHKIQLFENA | | | |
| | | RHLENTRRQLRKWQRRMARRHVR | | | |
| | | GLRSHEQSQGWRDARDQVARLHAL | | | |
| | | VAARRASSQHLVTKRLVTQYAHVAL | | | |
| | | EDLRVKSMTASARGAVESPGRNVRA | | | |
| | | KAGLNRAILDVGFGEIRRQIEYKAVL | | | |
| | | NGTRVTVVDPAYTSQTCNRCGHVD | | | |
| | | AKSRRTAISSPAPTAATPLTPTIPSRSC | | | |
| | | WRRSSPSSPPTSPVSRSRSTRRCTAY | | | |
| | | TSRICVTTLQTSSSPGSPPWTPSGSRS | | | |
| | | PTPTKAGDSAAPFVAFGRLRTFTPKG | | | |
| | | CCRRLSSGHFPCMGGVLRAEPVWV | | | |
| | | ETFTGLRMDRFVKLVKVVRERGGN | | | |
| | | GPGGGRPWCLPLPDRVLLVAVYYRT | | | |
| | | NLTMRQLAPLFGISPATVCRVIQRLR | | | |
| | | PLLALERAPQPVVDTERLWIVDGTLI | | | |
| | | PVRDRKVGASSRNYRFSANVQVIID | | | |
| | | ADTRLVIAAARPAPGNKADAHVWR | | | |
| | | GSDLPALAAGTTVIADGAYLGTGLI | | | |
| | | VPHRKRAGRPLLRGQEEDNAEHRR | | | |
| | | VRARVEHTFARMKNWKILRDCRQK | | | |
| | | GDGLHHAIQAAATMHNLAMTR | | | |
| DDE_Tnp_I | CAG8582489.1 | FWSQKAKEISEKLWLPDKHDLKKS | 1473 | ISXO2-like | Scutellospora |
| S1595 | | NLNSHSWFNIKKLEQVFLKNVEVPII | | transposase | calospora |
| | | SRQQQNIIDSEDDRLKCRKIRIYPNK | | domain | |
| | | KEKQTLKKWIGDARWTYNQCLDSL | | | |
| | | NDIRDMKTKKEKIQYMRQKHIVLET | | | |
| | | PKAIRDAAMMDLFKNIKSNHILKRA | | | |
| | | RFILKKRRKKDVNQSITIQVENIRCK | | | |
| | | TGMYSFVKKIKTKEKIPEIKHALNIV | | | |
| | | MNRLKHFYICISIDIEKHEIVNEDIISL | | | |
| | | DPGVRTFMTGYDPKGNILEFGNKDI | | | |
| | | DKIQEKCQRYDKLQSCMNQELDKR | | | |
| | | LSSQDCEIYLSELPDCVSMLTWSHY | | | |
| | | KFKMFLRHKVREYPDMNMIEYTEE | | | |
| | | YTSKTCTRCGIINEKLGGSKNFNCGS | | | |
| | | CGLKIDRDHNAERITLFLDNITKIIKN | | | |
| | | KSDKENKDMNDDDYLSEGEKRVFL | | | |
| | | KIVEKRDKETIKKIIEDHVEKGSIVH | | | |
| | | TDCWGGYLGIEDLGVAHETVNHSK | | | |
| | | NFTDPETGVNTNMIEGLWNGIKLQI | | | |
| | | APRNRNKNLINDHLLEYIWRRINKD | | | |
| | | KLWEAFIYALRSTAYYDNK | | | |
| DDE_Tnp_I | CAG8447381.1 | MNLYKLLISLLVLQIIFVVLFSNICLA | 1474 | ISXO2-like | Scutellospora |
| S1595 | | DSDEKNDGNKKENKNLSPNFDITKA | | transposase | calospora |
| | | KEISERLWLPEKHDLKISNFISHSWF | | domain | |
| | | NIKRSEQVSIKNVEIPIIPRYQEHIDSE | | | |
| | | NDELKTKKIRIYPTKEEKQKLKSWI | | | |
| | | GTARWTYNQCLDSLDEIRNLKTKKE | | | |
| | | KIQYMRQKHIVASNYKNTELSWVID | | | |
| | | TPSSVRDAAMMDLFKNIKSNHARKI | | | |
| | | ARFTLKKQKKKDKNQSITIEHLWFN | | | |
| | | KSKIFSFLRNIKTKEKIPEIKHAVNIIM | | | |
| | | NRLKYFYICIPIPTNINKHESINEDVL | | | |
| | | TLDPGVRTFMTGYDTKGNISEFGNK | | | |
| | | DIDKIQIRCLRYDKLQSCLNQESDKG | | | |
| | | LSLQDSEISMSELSDCVTARNMLTW | | | |
| | | SHYKFKMFLRHKIREYLDMNLIECT | | | |
| | | EEYTSKTCTRCGMINKLGGSKNFTC | | | |
| | | GSCGLKIDRDHNEKRDSETIRKIIED | | | |
| | | HVEKGSIVHTDRWKGYLGIENLGVT | | | |
| | | HKSVNYSKNFTDPITGVHTNMIEGL | | | |
| | | WNGIKLQIALRNRNKNLIKDHLLEFI | | | |
| | | WRRINKDKLWDAFIYALQSTAYYEN | | | |
| | | K | | | |
| Phage_ | WP_161234386.1 | MARPRAKKKKVSSGTHTRSVFLYG | 1475 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1476 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1477 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1478 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1479 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1480 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1481 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1482 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Phage_ | WP_161234386.1 | MARPRAKKKKVSSGTHTRSVFLYG | 1483 | Phage | Blautia wexlerae |
| integrase | | SPNAEKRSTLEKLQADYTDAVNFYI | | integrase | |
| | | SLLSDREECLLQLLQNDKKDPLLRK | | family | |
| | | LEKESRIEGLSSAYSQNAFDEAVTKL | | | |
| | | HNRLDNIRKDVIAATGGSVFAVSILL | | | |
| | | FHAVLSGQSREEMCGMLARIRDSYK | | | |
| | | AKEKIQYYDKLHDTVKTMEEKEFL | | | |
| | | DSVSEVAMFYHIISDEYRIPVVKKAH | | | |
| | | VLSVTIPDRKRERMQVPVQADRDA | | | |
| | | LRRMEQYGVSGSMRYTITDGGSLKL | | | |
| | | TCSFEKKTRTPEEHSAVIGVDVGITD | | | |
| | | AFHTSEGQAIESFQPVIEFYQTEVEP | | | |
| | | AFGKLSTLRNRKQQLRRFLKKHKG | | | |
| | | VLPEKVILNLRKRIDHLEKDIRQAHA | | | |
| | | PYRRKRHYYQLVEYTVRNAVNTYIE | | | |
| | | SLNGDKTVLTAMELLDIKEFNKSRR | | | |
| | | VNGMLSDFSRGKLAEKLMEELSWH | | | |
| | | GFPFVQVEPAYTSQICPVCGCLDKAS | | | |
| | | RIIHKVFINGLVKNAIAELTEHTAELR | | | |
| | | KESGLKELFLCRIKSQNNKIAPYTET | | | |
| | | HWNDKKLRYFIERHDIRDNKGDLYP | | | |
| | | LTSHQFRSTFVRELIKRKVPIAMIMK | | | |
| | | QYSHVSIEMTAHYLTLQEEEVKEIYS | | | |
| | | DMILSPESKIAGLRAKEIKGKLDDLF | | | |
| | | HGKTEDEIDDVISGLAKTMSFNPLPT | | | |
| | | GVCLYDFRRGNCTDGDGCFFYNCP | | | |
| | | NYITEVQFYPILKDELDLLEKEMVR | | | |
| | | LKELGQEPAYQVQAVKYKYLKPLVE | | | |
| | | SLEVQLNGKESVG | | | |
| Resolvase | MBX8642660.1 | MKLSDRARKNGIDYRTAYRLYRSGR | 1484 | Resolvase, N | Thermoplasmata |
| | | FPGPTGQLATGTKLVHEPEPGHAPAE | | terminal | archaeon |
| | | RVVLYARVSSADRKSGTGRQMKRL | | domain | |
| | | EDYAAARGSHAGAEISEIGSGLNRL | | | |
| | | KKGSLSWMYEISKCAPQEALRDLDS | | | |
| | | AFTRFFDGNADFPKFKSKKHGCGSF | | | |
| | | RLTGAMKAQGYSIQLPCIGTMSLKE | | | |
| | | NGCLPADGHIPSTTVSERGGRWFVS | | | |
| | | LAVIEEHTVPENSGSICGVDLGVKNL | | | |
| | | ATVSDGTVFENPRSLSTYIRKLKGQ | | | |
| | | QREVSRKVKRSNSRRKAVHRLNRT | | | |
| | | HLKISYMRMDAIHKATTWLAKNKS | | | |
| | | AIVIEDLNAGGMMCNHRLAAAISDA | | | |
| | | SFGEFRRQLEYKAGWYGSRIVVADR | | | |
| | | FYPSSRTCSACGHVKQELKLSERVFE | | | |
| | | CEMCNSMIDRDLNAAINLSGLAASS | | | |
| | | AESLNACLR | | | |
| Resolvase | RHZ50278.1 | MLDPLKSNFNKHGSVSSSHFLTKTT | 1485 | Resolvase, N | Diversispora |
| | | FLTFLTKPPFYNLAQLNTTYQSAHKI | | terminal | epigaea |
| | | QETYDVSVETLRRWADSGRIAIVRTP | | domain | |
| | | GGKRLYSITNIQEIFRDNQQTQITQK | | | |
| | | AKICYAKVSSEHQRDDLERQIANLR | | | |
| | | QYYPEYEIISDIGSGLNWKRRGTDC | | | |
| | | ADLDPSLWNGSLRKMGLSSWYSVR | | | |
| | | ISVLKVVKPENLRKTYSQSSRYLWQ | | | |
| | | DIMECVPPPIVEEDEKLPKPKKNKSS | | | |
| | | KIPAGKTQRIRLFPTQEEKSKLKRW | | | |
| | | MGTARWTYNRCLVAVEKEGIERTKK | | | |
| | | ALRAQCLNAANFNNTELQWVLETP | | | |
| | | YDIRNEAINDLLKSYSSNFAAKRKK | | | |
| | | FKMKFRFKKDQQQSIAILSKHWDKS | | | |
| | | KGVYTFLCKIKSAENLPAELHYDSR | | | |
| | | LVMNQLGEFYLCIPQPLEIWAENQG | | | |
| | | PIQSDAVIALDPGVRTFITIQVDKL | | | |
| Resolvase | MBO5650323.1 | MNTSNITNYKPKEFAELLNVTVKTL | 1486 | Resolvase, N | Selenomonas sp. |
| | | QRWDREKTLVANRTPTNRRYYTYD | | terminal | |
| | | QYLQFKGIGKDADFRKIVIYTRVSTR | | domain | |
| | | NQTDDLENQVDFLQTYVNAKGLIA | | | |
| | | DEIIRDYGSGLNYNRKKWNQLLGE | | | |
| | | VMENKVKMILVSHKDRFVRFGFDW | | | |
| | | FEKFCNKFNVEIVVVKNEKLSPQKE | | | |
| | | LVQDIVSILHVFSCRLYGLRKYKKQI | | | |
| | | EGMRILLKAFRTEIAPTNEQKMKIIR | | | |
| | | SIGVARFLYNQYIAYNRHLYKMYQR | | | |
| | | GILDEKQKHFVTANDFDKYVNHKL | | | |
| | | KKELPWIDQCGSKARKKALVNAEQ | | | |
| | | AFRRFFSGTSGFPNFKKKVNQDVKL | | | |
| | | YFPKNNKGDWTIWRHKLMIPTLKQ | | | |
| | | VRLKEFGYLPVGAKVTNGTVSYMA | | | |
| | | GRFYVSVVVDIDEKSKYNKDLEASY | | | |
| | | HTVTEGVGIDLGVKDLAIVSDGKVF | | | |
| | | KNINKSSKVKRLE | | | |
| Resolvase | EFH82150.1 | MYSAAQFAKQVGVSVKTLQRWDR | 1487 | Resolvase, N | Ktedonobacter |
| | | EGRLKAKRTLSGRRYYDEADLATAL | | terminal | racemifer |
| | | NLPKPPAIRRTVAYCRVSSPAQRPDL | | domain | DSM44963 |
| | | QNQRAALERYAVSKQLVVDEWIVEI | | | |
| | | GGGLNFERKRFLRLVDAIVEGEVSC | | | |
| | | LLIAHQDRLARFGFALIKHLCETHHT | | | |
| | | ELVVMNTQTLSPEQELVVQDLMSII | | | |
| | | HGFSSRLYGLRNYRKALEKALKDEN | | | |
| | | RAQDQDDPTPEQVEYLKRACGTRR | | | |
| | | FIYNWGREQWEKQYQAYKLEQETV | | | |
| | | FEEQRVLTPPNTFALKKQFHQIREQD | | | |
| | | YPWTYQVTKCVVEGAFADLKSAYD | | | |
| | | NFFAGRSNYPQYKKKGKSHESFYLS | | | |
| | | NDKFTVGTHWISIPGLGRFILDQRQT | | | |
| | | KKDRGKLLRRLGAVNVAEKLRFVE | | | |
| | | KGKATTPAKKRNKRKQVVCERVKIL | | | |
| | | GATVSCEAGHWYVSIQVEIKKQRPL | | | |
| | | TPTAVVGWMSD | | | |
| Resolvase | MCL4317383.1 | MKLSDRARKNGIDYRTAYRLYRSGR | 1488 | Resolvase, N | Candidatus |
| | | FPGPTGQLATGTKLVHEPEPGHAPAE | | terminal | Thermoplasmatota |
| | | RVVLYARVSSADRKSGTGRQMKRL | | domain | archaeon |
| | | EDYAAARGSHAGAEISEIGSGLNRL | | | |
| | | KKGSLSWMYEISKCAPQEALRNLDS | | | |
| | | AFTRFFDGNADFPKFKSKKHGCGSF | | | |
| | | RLTGAMKAQGYSIQLPCIGTMSLKE | | | |
| | | NGCLPADGHIPSTTVSERGGRWFVS | | | |
| | | LAVIEEHTVPENSGSICGVDLGVKNL | | | |
| | | ATVSDGTVFENPRSLSTYIRKLKRQQ | | | |
| | | REVSRKVKRSNSRRKAVHRLNRTHL | | | |
| | | KISDMRMDAIHKATTWLAKNKSAIV | | | |
| | | IEDLNAGGMMCNHRLAAAISDASF | | | |
| | | GEFRRQLEYKAGWYGSCIVLADRF | | | |
| | | YPSGRTCSVCGHVRQELKLSERIFEC | | | |
| | | EMCNSMIDSDLNAAINLSGLAASSA | | | |
| | | ESLNACLRLEVAELLAQCPPVIQEM | | | |
| | | NTISLGMSG | | | |
| Resolvase | CKN51115.1 | MNLAAWAERNGVARVTAYRWFHA | 1489 | Resolvase, N | Mycobacterium |
| | | GLLPVPARKVGRLILVDELASEAGA | | terminal | tuberculosis |
| | | QPKTAVYARVSSADQKSDLDRQVAR | | domain | |
| | | VTSWATAEQIPVDKVVTEVGSVLNG | | | |
| | | HRRKFPAVLRDLSVTRIVVEHRDRF | | | |
| | | CRFGSEYVHAALAAQGRELVVVDS | | | |
| | | AEVDDDLVWDMTEILTSMCARLYG | | | |
| | | KRAAQNRASGPSRLPLSMIMRRPEM | | | |
| | | PRLEIPNGWCVQAFRFTLDPTAEQA | | | |
| | | HALARHFGARRKAYNWTVAQLKA | | | |
| | | DIQAWRATGAQTAKPSLRVLRKRW | | | |
| | | NTVKDEVCVNAETGTVWWPECSKE | | | |
| | | AYADGIAGAVDAYWNWQQRRAGK | | | |
| | | RDGKRMGFPRFKKKGRDADRVSFT | | | |
| | | TGAMRVEPDRRHLTLPVIGCVRTHE | | | |
| | | NTRRIERLIAKDRARVLAITVRRNGT | | | |
| | | RLDASVRVLVQRPQQPNVELPESRIG | | | |
| | | VDVGVRRLATVATADGACCPVLVPD | | | |
| | | G | | | |
| Resolvase | COW46299.1 | MNLAAWAERNGVARVTAYRWFHA | 1490 | Resolvase, N | Mycobacterium |
| | | GLLPVPARKVGRLILVDELASEAGA | | terminal | tuberculosis |
| | | QPKTAVYARVSSADQKSDLDRQVAR | | domain | |
| | | VTSWATAEQIPVDKVVTEVGSVLNG | | | |
| | | HRRKFPAVLRDLSVTRIVVEHRDRF | | | |
| | | CRFGSEYVHAALAAQGRELVVVDS | | | |
| | | AEVDDDLVWDMTEILTSMCARLYG | | | |
| | | KRAAQNRAKRAVAAXXXXXXXXX | | | |
| | | XLPLSMIMRRPEMPRLEIPNGWCVQ | | | |
| | | AFRFTLDPTAEQAHALARHFGARRK | | | |
| | | AYNWTVAQLKADIQAWRATGAQTA | | | |
| | | KPSLRVLRKRWNTVKDEVCVNAET | | | |
| | | GTVWWPECSKEAYADGIAGAVDAY | | | |
| | | WNWQQRRAGKRDGKRMGFPRFKK | | | |
| | | KGRDADRVSFTTGAMRVEPDRRHL | | | |
| | | TLPVIGCVRTHENTRRIERLIAKDRA | | | |
| | | RVLAITVRRNGTRLDASVRVLVQRP | | | |
| | | QQPNVELPESRIGVDVGVRRLATVA | | | |
| | | TADGACCPVLVPDG | | | |
| Resolvase | MCS5695294.1 | MKGVSELLNMTRQPLSSSSRNTSAR | 1491 | Resolvase, N | Desulfofundulus |
| | | RRWSYSGHDQKADLQRQVEVLKG | | terminal | thermocisternus |
| | | AYGSKFSDVVVLTDVGSGLSTNRRS | | domain | |
| | | LRKAMQMARERKIRAVAVTYPDRLT | | | |
| | | RFCFEYLKEYFNSFGVEVLVLNREE | | | |
| | | DRSPQQELVEDLLTIVTSFAGKLYGH | | | |
| | | RSHAELQEKLAAGGTLCAPDLRLK | | | |
| | | VKPGGELVALLDVKVDVPEEKPSGD | | | |
| | | PGRALSVDWGLRKLVTCTVVSRKG | | | |
| | | QLTPPFFVFWSGLKARLFRIREDIKK | | | |
| | | LQKERDRYEKGTPDWKKYNRKIAA | | | |
| | | AWQKYHRVQHTLAHAVSTLLVLLA | | | |
| | | RAFGCRHIFIEWLVTLHGKKGRNRD | | | |
| | | LNWWVSTVVRGLLFRLLRYKAKLA | | | |
| | | GIRVFMVPPGGTSRVCPRCLGAGKH | | | |
| | | VISPGNKAEKDSGSWFVCPSCGWQ | | | |
| | | ADRDYAGSLNIARVGFNLARPLSYK | | | |
| | | VGGAAMPFPSRVASAEVLREAMTFT | | | |
| | | TTLGYTCSVFPGNIGCLVKLLDLQC | | | |
| | | RT | | | |
| Resolvase | WP_235657201.1 | MAYVPLREAVKRLGLHPNTLRKYA | 1492 | Resolvase, N | Fischerella |
| | | DNGKIESIKNEAGQRLFNVESYLRG | | terminal | thermalis WC439 |
| | | ATRASIVCYCRVSSTKQRDDLDRQI | | domain | |
| | | AYMQSLYPEAEIIKDIGSGINFKRKG | | | |
| | | LQTLLDRLMRGDKLTLVVACRDRLC | | | |
| | | RFGFELPRSTWSSKTVDKSWFSKTL | | | |
| | | YTVPNQNLPRIFSPSSTSSLVECTDSG | | | |
| | | NTVKKSRKIRIFLKATQKQIIKQWFG | | | |
| | | VSRFVYNTTVKLLQDSTIKANWKA | | | |
| | | VKTDILNGLPDFCKSVPYQIKSIAIK | | | |
| | | DACKAVSNAKKKFKNGGGISKVKF | | | |
| | | RSRKDPIQSCYIPKSAVSDKGIYHTIL | | | |
| | | GEVTFKEALPQSFGDCRLVKAYGDY | | | |
| | | YLTVPEEVSRQQSENQGRVVALDPG | | | |
| | | IRTFITFFSESSFGEIGISANIQIQKLCF | | | |
| | | RLDKVISKIAKAKCKSKRRLKLAAT | | | |
| | | RLRGKIKNLVDELHKKTARFLVDNF | | | |
| | | DVILLPTFETSRMSKKAKRRIRSKSV | | | |
| | | RQMLTLSHYRFKQFLKHKAFETSKV | | | |
| | | VMDVNE | | | |
| Resolvase | RKU31134.1 | MKHYKIPRETSAILGISIDRLRRLAE | 1493 | Resolvase, N | Candidatus |
| | | NGTISTIGTPGGQKHYDVQGYLDEQ | | terminal | Poribacteria |
| | | TGTDITTIGYCRVSGKGQAEDLASQ | | domain | bacterium |
| | | VAYLQKHYPEAEIIKDFGSGINFKRK | | | |
| | | GLRTLLERLLRGDKLRVVVAHRDRL | | | |
| | | ARFGGEAKDTPKNFKGLVPIVFDQL | | | |
| | | PEWHVETPRQIKAGAVIDACQAIKN | | | |
| | | AKVKCKETDKFQKVSFRSRKNPRQ | | | |
| | | TLYLRADSLKKNGFYVRLLGQMKM | | | |
| | | SEPLPAKPQGTGKVSERDTNAEVKD | | | |
| | | SQLIMENGRYFLCVSYVEKKKTREP | | | |
| | | SGRIVALDPGVRDFMTFFSEDRFGW | | | |
| | | LGQQCINRIQRLCQHCDNLLSRATQ | | | |
| | | EQRPLRRALRKAANRIKVKIRNLIDE | | | |
| | | LHKKIAHFLVTNFDIILLPTFETKQM | | | |
| | | TKRGARKLRKKSVRQMLTLSHYRF | | | |
| | | KVFLKHKAKEYGAQVIDVCEAYTS | | | |
| | | KTVSWTGELITNLGGSRVIKSSEGHR | | | |
| | | MDRDLNGARGIFIKNVARALTVRPC | | | |
| | | TANLGASKSLIPSAT | | | |
| Resolvase | CAG8682803.1 | DLERQIANLRQYYPEYEIISDIGSGL | 1494 | Resolvase, N | Ambispora |
| | | NWKRRGFVALLERIHTEGIEEVVVT | | terminal | gerdemannii |
| | | RKDRLCRLGSELVEWIFEKNGTRLV | | domain | |
| | | VLGMDVSAESSEAGELAEDLLSIVT | | | |
| | | VFVARHNGMRSAANRRRRREVAKA | | | |
| | | QEEQELQDSSRQDTTYPSLSYARGE | | | |
| | | VETQTLDGNSAMDLQSGIERTKKAL | | | |
| | | RAQCLNAANFNNTELQWVLETPYD | | | |
| | | IRDEAMNDLLKSYSSNFAAKRKKFK | | | |
| | | MKFRSKKDQQQSIAILSKHWGKSK | | | |
| | | GVYTFLCKMKSAENLPAELHYDAR | | | |
| | | LVMNRLGEFYLCIPQPLEIWAENQG | | | |
| | | PTQSDAVIALDPGVRTFITGYDPSGQ | | | |
| | | AVEWGKNDISRIYRLSHIYDKIQSTH | | | |
| | | DSIHGKVHKRKRYKLRRVMLRIHK | | | |
| | | KIRCLINDCHHKLAKWLCQSYRIILL | | | |
| | | PEFKTQGMVRRGKRRIRSKTARMM | | | |
| | | LTWSHFRFRQYLLHKVREYPWCRVI | | | |
| | | ICTEGYTSKTCGCCGHIHRKLGGSK | | | |
| | | VFRCPSCTAELDRDINGARNILLRYL | | | |
| | | TVTSKEP | | | |
| Resolvase | CAG8436474.1 | MTSKYKPAEKIKKIYGVSTSTLRRW | 1495 | Resolvase, N | Scutellospora |
| | | NDKGDVAYITMPGGKRLYSTDDIDN | | terminal | calospora |
| | | IFGRESTQKKKICYARVSSEKQKDDL | | domain | |
| | | GRQRAYLVSEFPDHEIITDIGSGLNW | | | |
| | | KRRGFTSLLERVYARDIEEVVVTRK | | | |
| | | DRLCRFAYELVEWIFSKHDVKLVVL | | | |
| | | GSDVGSNDPDTGELAEDLLSIVTER | | | |
| | | EKLKKWMGTAKWTYNRALDLIKN | | | |
| | | GESRTKKNLRQKCIKVENFRHENTW | | | |
| | | VLETPYGVRDEVLIELLEAGEYAFL | | | |
| | | KNIRTSEKLPDINHAVNIIRDSFKRFY | | | |
| | | VCVPVPIKEQYRENDDFISIDPGVRT | | | |
| | | FMTGYDPKGKIFEYGKGDISRVYRL | | | |
| | | CKRHDEYQSQRSVLKGGSNKRERY | | | |
| | | KLKRKMLKIHDKIKNLIRDCHHKIV | | | |
| | | KELCENYNTILLPRFETKNMVRKKD | | | |
| | | KELDNQESKSIKNHKKKFKRKIKSK | | | |
| | | TARMMLTWSHHRFQQHLVHKIREY | | | |
| | | PGRLLILCDEHYTSKTCGNCGYIKH | | | |
| | | NLGGAKIYRCNRCGFEIDRDHNGAR | | | |
| | | NILLKHLSQRDLTLGPTPFE | | | |
| Resolvase | WP_102164839.1 | MTYVPLREAVKRLGLHPNTLRKYA | 1496 | Resolvase, N | Fischerella |
| | | DNGKIESIKNEAGQRLFNVESYLRG | | terminal | thermalis WC441 |
| | | ATRASIVCYCRVSSTKQRDDLDRQI | | domain | |
| | | AYMQSLYPEAEIIKDIGSGINFKRKG | | | |
| | | LQTLLGRLMRGDKLTLVVACRDRLC | | | |
| | | RFGFELPRSIWSNKTVDKSWFSKTL | | | |
| | | YTVPNQNLPRIFSPSSTSSLVECTDSG | | | |
| | | NTVKKSRKIRIFLKATQKQIIKQWFG | | | |
| | | VSRFVYNTTVKLLQDSTIKANWKA | | | |
| | | VKTDILNGLPDFCKSVPYQIKSIAIK | | | |
| | | DACKAVSNAKKKFKNGGGISKVKF | | | |
| | | RSRKDPIQSCYIPKSAVSDKGIYHTIL | | | |
| | | GEVIFKEALPQSFGDCRLVKAYGDY | | | |
| | | YLTVPEEVSRQQSENQGRVVALDPG | | | |
| | | IRTFITFFSESSFGEIGISANIQIQKLCF | | | |
| | | RLDKVISKIAKAKCKSKRRLKLAAT | | | |
| | | RLRGKIKNLVDELHKKTARFLVDNF | | | |
| | | DVILLPTFETSRMSKKAKRRIRSKSV | | | |
| | | RQMLTLSHYRFKQFLKHKAFETSKV | | | |
| | | VMDVNEAYTSKTVSWTGEIIHNLGG | | | |
| | | AKFIKSPTDGRVMPRDLNGARGIFL | | | |
| | | RALVDTPSLRECIC | | | |
| Resolvase | GHD59043.1 | MPVPVVQTPSVIWPVEEPTPEVVGR | 1497 | Resolvase, N | Streptomyces |
| | | TVAYCRFSSGDQKAELDRQVSRVVQ | | terminal | mirabilis |
| | | GAIGLGLAVAEVVTEAGSGLNGNRR | | domain | |
| | | KLHRVLSDPAVAVIVVEHRDRPARFG | | | |
| | | VEQLESALSASGRRLVFEARPGFHIV | | | |
| | | GHRLALDPNASALQALASHCGAAR | | | |
| | | VAYNWAVRHVPANWSQRAAEETYG | | | |
| | | VPEAERVAWRSWSLPSLRKAFNEAE | | | |
| | | HNDPCLREWWAQNSKEAYNTGLAN | | | |
| | | AAAAFDNYAKSRRGERKGTRMGRP | | | |
| | | RFKSKRKARPACKFTTGTIRLDDRR | | | |
| | | HIVLPRLGRIRLHEDVQPLVDAIAEG | | | |
| | | GTRILSVTVRFERGRWFAVLQTEERP | | | |
| | | TIAPATRPYTAVGIDLGVKTLLVMAD | | | |
| | | SAGEVREVANPKDYDQTLTQLRKAS | | | |
| | | RTVSRRRGPNRRTGQAPSRRWEKA | | | |
| | | NAVRNRVHHRVANLRENHLHQATA | | | |
| | | RIPAEYGTVVVEDLNVKGMVRNRR | | | |
| | | LSRRISDAAFGELRRRLTYKTQRHG | | | |
| | | GCLIVADRWMPSSKTCSRCGVVKA | | | |
| | | KLSLGVRVFERAVGAAPRQGTELDR | | | |
| | | TPSASNSRSGDTVTNLPGGNTRNAE | | | |
| | | SR | | | |
| Resolvase | GAQ87932.1 | MGYVSLSSARHHFGVSGKTLYRWE | 1498 | Resolvase, N | Klebsormidium |
| | | ASGKVTVKRSPGNRRLFLVDDPQPQ | | terminal | nitens |
| | | YEPTRESVVYVRVSSSKQQDDMRR | | domain | |
| | | QEKYLLDQHPGSRVVRDIGSGLNFK | | | |
| | | RKGLLSLLERSEEGLLRRVVVASED | | | |
| | | RLCRFGFDLLAWQFKRHGVELLVC | | | |
| | | DKADKSPEAELAEDVLAVIQVFGCR | | | |
| | | WNGKRSYNRAVSLMHETSVYNKM | | | |
| | | RLRNMITPAEVNQDNLWLLETPKDI | | | |
| | | RAGAVFEAAKNIKAAFTNKARGNIE | | | |
| | | NFKMGYRSRKKEDSCGWTINVPKT | | | |
| | | ALKVKGERCLQVYRDSCPWLFQTL | | | |
| | | GRIGPLSMDCKIQFDGMRYFLIVPYE | | | |
| | | RHVVSPRREGVIALDPGVRTFQTAY | | | |
| | | SPDGHCYELGRSCCRRLEALCVHLD | | | |
| | | RLISLASVTPKTLRRTKWAMGLRIK | | | |
| | | KLRRRLQSLRDEMHYKAADYLTRT | | | |
| | | AHIILLPTFETKDMTRRTTRRLRSRT | | | |
| | | VRNLSLLSHYKFKQRLIAKAAVRGV | | | |
| | | KVLQVSEHYTSKTCGRCGSIHETLG | | | |
| | | GRRVFKCPRCGVVLDRDCNGARNV | | | |
| | | LLRAMREEPPSRGAMQDGATVDMP | | | |
| | | CLAWTNE | | | |
| Resolvase | KAJ3080465.1 | MTVVAVVEFFLHIQPFSKPATARVAL | 1499 | Resolvase, N | Quaeritorhiza |
| | | SAATLPPSNPKQSQVEKEPDGTDQD | | terminal | haematococci |
| | | TDQGTEQGTAKQPHPVLPRRVHIHL | | domain | |
| | | GAVERLPVHRLGDYFEDVVRWACR | | | |
| | | VHLNLNVENIQWTIPAEIRSVCATID | | | |
| | | HWGLGQPVHVQPSRDMQYRYKLTP | | | |
| | | LNATRTLFDASVQYSRPMDIGSGLN | | | |
| | | FKRKGLKRLLERVMSGTVEEIVVTH | | | |
| | | KDRLCRFGFELIEFMVNKFGGRILV | | | |
| | | QNHAVRTPEEELTQDLLTIIHRKVCS | | | |
| | | ATDCDEPIDPATNGYLCEAHYDPGA | | | |
| | | SQCQGVAMTGRNKGQRCSYASSAG | | | |
| | | FCSHHAKKATKEKVKENENVTCRL | | | |
| | | YRLRPNKTQVKLLRQWFGVAREYY | | | |
| | | NATIEYLVKEKAKACFQDIRPIIKDK | | | |
| | | LDNAKPYRLQVPDKIRQGAIQDACQ | | | |
| | | AMNNAKLKYQQNQVFSKLSFRTRR | | | |
| | | DPSQSIYLDKSAIKVEAEAVVFYPEI | | | |
| | | TKAYLEEARSKDDKSEIDSEIRTTEA | | | |
| | | VEINRACRIVLKHNKYFQLASPRSQ | | | |
| | | AVQQGPTYGECVALDPGGRSFLTLY | | | |
| | | SPDLCGHLSYEPRKRIEATYNEYDK | | | |
| | | V | | | |
| Resolvase | GAQ92077.1 | MTNEQRYLGTHAAKQVLGVCGATL | 1500 | Resolvase, N | Klebsormidium |
| | | RQWADSGLITSFKTPGGKYRYLIDN | | terminal | nitens |
| | | VISGGSADTRAEELQRPATEKQKVC | | domain | |
| | | YCRVSSLGQKADLDRQAQYMQSRY | | | |
| | | PEHTIIRDIGSGINFKRKGLQTILELA | | | |
| | | FRGRLEEVVVAYRDRLCRFAYELVA | | | |
| | | WIFHQHGVRLVVLNEEEGRTILKRW | | | |
| | | FGCARKTYNMALEALKKRTAHKRT | | | |
| | | EAWLKNRFTRASNVSKANSFLLDTP | | | |
| | | THVRDGAIADLMKGLRNEVAKKRK | | | |
| | | NGSHAFEMRYRSKKEVQSLYLEKTA | | | |
| | | IKKLIHADSHSDFLSMYPTHVTNAIF | | | |
| | | LISKKASLLNCRGKVSFDARLVMDK | | | |
| | | LGHFHIHATVEKDEIPSENQGRGIVA | | | |
| | | LDPGVRTFMTAYSPSDNVAFQIAPGC | | | |
| | | IQRMVRLEHHIDRLRSEISLLPPQCK | | | |
| | | GRARRMRKAALRLHKRVSNLTSEV | | | |
| | | HWKVAQFLTDRFQTIVLPPFATQEM | | | |
| | | CSRTGDRQRRIGKSTSRKMHLWGH | | | |
| | | YTFRTRLAMKCKERGCDLKVLDEV | | | |
| | | YTTKTCTGCGWVHDTIGGSKVFLC | | | |
| | | QGCGLETDRDINGARNIFLKHHKRL | | | |
| | | MGGSLPS | | | |
| Resolvase | MCI8748163.1 | MKYYSIGEFANKIGKTIQTLRNWDK | 1501 | Resolvase, N | Lachnospiraceae |
| | | SCSLKPHHITKAGTRYYSQEQLNHF | | terminal | bacterium |
| | | LGLKPQDKLNKKTIGYCRVSSYKQK | | domain | |
| | | DGLERQIENVKTYMYAKGYQFEIIS | | | |
| | | DIGSGINYNKKGLNKLLDMVTNSEV | | | |
| | | DKIVVLYKDRLIRFGYELIENLCEKY | | | |
| | | GTAIEIIDNTEKTEDQELVEDLIQIVT | | | |
| | | VFSCRLQGRRAEKARLYPSELQEQK | | | |
| | | LWQSVGTARFIYNWTLARQEENYK | | | |
| | | NGGKFISDKVLRKELTQLKKSELSW | | | |
| | | LNEVSNNVSKQAVKDACNAYKRFF | | | |
| | | KGLSGKPKFKSKRKGKKSFYNDNIK | | | |
| | | LKVKESKLVSIEKIGWIKTNEQLPAG | | | |
| | | VKYSNPRINYDNKYWYISVGAEQEE | | | |
| | | IKEDLTDISLGVDLGLKNLAICSSGT | | | |
| | | VYKNINKTYTVRKIEKRLKKLQRQV | | | |
| | | SRKYEQNKKGKEYVKTKNIIKLEKQ | | | |
| | | IQQVDRRLANIRNNYLHQTTTSIVKT | | | |
| | | KPYRVVIEDLNVKGMMKNKHLSDA | | | |
| | | IRKQGFYEFRRQLGYKCKFRGIELV | | | |
| | | VADRFYPSSKTCSQCGKINKDLKLK | | | |
| | | DRVYSCSCGLSIDRDLNACINLSRYK | | | |
| | | LA | | | |
| Resolvase | RHZ85702.1 | MYMLKSNFNKHGSVSSSHFLTKTTF | 1502 | Resolvase, N | Diversispora |
| | | LTFLTKPPFYNLAQLNTTYQSAHKIQ | | terminal | epigaea |
| | | ETYDVSVETLRRWADSRRIAIVRTPG | | domain | |
| | | GKRLYSITDIQEIFRDNQQTQITQKA | | | |
| | | KICYARVSSKHQRDDLERQIANLRQ | | | |
| | | YYPEYEIISDIGSGLNWKRRGTDCA | | | |
| | | DLDPSLWNGSLRKMGLSSWYSVRM | | | |
| | | SVLKVVKPENLRKTYSQSSRYLWQ | | | |
| | | DIMECVPPPIVEEDEKLPKPKKNKSS | | | |
| | | KIPAGKTQRIRLFPTQEEKSKLKRW | | | |
| | | MGTARWTYNRCLVAVEKEGIEQTKK | | | |
| | | ALRAQCLNAANFNNTELQWVLETP | | | |
| | | YDIRDEAMNDLLKSYSSNFAAKRK | | | |
| | | KFKMKFRSKKDQQQSIAILSKHWG | | | |
| | | KSKGVYTFLSLRAQCLNAANFNNTE | | | |
| | | LQWVLETPYDIRDEAMNDLLKSYSS | | | |
| | | NFAAKRKKFKMKFRSKKDQQQSIAI | | | |
| | | LSKHWGKSKGVYTFLCKMKSAENL | | | |
| | | PAKLHYDSRLVMNRLGEFYLCIPQP | | | |
| | | LKIWAKNQGLTQSDAVIALDPGVRT | | | |
| | | FITGYDPSGQAVEWDKNDISRIYRLS | | | |
| | | HIYDKIQSTHDSIHGKVHKQKRYKL | | | |
| | | RKVMLIHVPKIDFGFLRYFDFLRFM | | | |
| | | QNLFYIWK | | | |
| Resolvase | KAJ3080568.1 | MRRGKQFYNVEDIERILGTKTKSEQ | 1503 | Resolvase, N | Rhizoclosmatium |
| | | LRGGQRPVCYACVSSSHQRKDLERQ | | terminal | hyalinum |
| | | IEDLKSRYPDAIVITDIASGLNWKRP | | domain | |
| | | GLNSLLELVHARSVSEIVVTHRDRL | | | |
| | | ARFGVNLLDWIFAKAGVKLVVLCG | | | |
| | | SADHQPTQYLELDNNGDRDGSTEN | | | |
| | | SGPTAAPGNAFNELAEDLLAITNFFV | | | |
| | | ARNNGLRAQAPTSDPEPSGSSSTTNP | | | |
| | | QKPKKAAPDRETHKQILVKWFGVT | | | |
| | | RFVYNQCVALSSSSNRVKPKRDSLR | | | |
| | | AAIINDEDYKSLMSKQNRKWLKEY | | | |
| | | HYDLKDEAICTYLKNLKSNFAKLAK | | | |
| | | GGQNKFCIKFKLRKDPVASILVLAK | | | |
| | | HYNKANNFFSPILNVRKMKSAEPLP | | | |
| | | VKLNWDSKLIRNQLGEYYLVFPQAI | | | |
| | | KKKSDSEEKEPRVVALDPGFKNFMI | | | |
| | | GYDPSGTVFSWGKQDIVRIGRLLHH | | | |
| | | KRNLHAKLSEVKDAKRNKRMKKA | | | |
| | | WLRMSKRIQDLVSEMHKKLALFLV | | | |
| | | QSYTHIYIPRLDFRHFKRIGKQYREK | | | |
| | | MATFSHCKFVDRLKDKVREYLNTK | | | |
| | | VFDKITEEYTSKTCTNCGCLDLNLR | | | |
| | | NKEVYSCIHCGTVIGRDFNGARNILL | | | |
| | | KTMKEVSALQIN | | | |
| Resolvase | GHO46160.1 | MCIENIYTPKEFGQLIGRTTNTLQKW | 1504 | Resolvase, N | Ktedonospora |
| | | DRKGLPKAHRSPTNRRYYTHDQYL | | terminal | formicarum |
| | | KYRGLKAAKQGLTMVYARVSSAAQ | | domain | |
| | | KPDLRNQVNALEAYCKQHSIAVDE | | | |
| | | WMFDIGSRLNYKRKHFNRLMEMVA | | | |
| | | LGQVRRLIIAHRDRLVCFGFEYFEAF | | | |
| | | CERHHTQILVINGDSLSPEQDLVQDL | | | |
| | | IAIVTIFSARLHGLRSYKKVLKDAAQ | | | |
| | | QKEEERMNRSHVIRLNPTPEQEVYF | | | |
| | | RKACGVARHAYNWALDHWKQARS | | | |
| | | EGKRVKMRELKAEYNRVKHEQFPW | | | |
| | | CADVTKCAPEQEFRHLGQAFDNYW | | | |
| | | RMKKDGTQPKLKHPRKDGEEAGFP | | | |
| | | HFKNKKRDRLSFYLNNDKFSVEGN | | | |
| | | WIRIPKLGKVNMTEQLRFSGKTLSA | | | |
| | | VVSERAGWWFVSIAVEVEHQAPTH | | | |
| | | QGDAVGIDLGIKTLVTLSDGEVFEN | | | |
| | | QKHYRQNLGRIKGLSKGLARKKEG | | | |
| | | SQNWWKNKKKLAKAHYRVANQRS | | | |
| | | DKLHKMTTRIALTYALIGLEDLNAK | | | |
| | | GMLANSCLAQAVNDASFFEVKRQL | | | |
| | | LYKAEQHGGYVQLVSRWYPSSKTC | | | |
| | | SSCGYVKPELLLSERVFICEDCGYVS | | | |
| | | DRDYNASLNIRNEASRLRTSVPVVA | | | |
| | | SSAR | | | |
| Resolvase | OGW52772.1 | MKIYRLNEFAKLIGKSVQTLQRWDR | 1505 | Resolvase, N | Nitrospirae |
| | | EGIFKAYRNKLNRRYYIHDQYLEYI | | terminal | bacterium |
| | | GQKASPEKKNIVYYRVSSSGQKGDL | | domain | RIFCSPLOWO2_ |
| | | ENQKKAIEQFCIAQGIAVSEWLSDIG | | | 02_42_7 |
| | | SGLNYTRKNFLSLMEMVERGEVAQI | | | |
| | | IIAHKGRVVRFGYMKKTIKNYCFNA | | | |
| | | TQSKLNELYEIALRYTSVKNEIFQRY | | | |
| | | GSISGLNYLSYPRQIRNEWVKTNYA | | | |
| | | NKFGLQARYWKQAVDEVFSNIKSN | | | |
| | | WSNGFRKIKNNIYKNKNYTEVEKH | | | |
| | | YAFYLLKASILLYKAITFQSFDLPEIF | | | |
| | | KDKDIRRDKIHKYLKSRLRKYLRTK | | | |
| | | SYQNKNRSFQIDRNMYDIHKDNKG | | | |
| | | RTWIGIMGLTPRKRVRLQMTSSTEST | | | |
| | | GNLRIVLKGKHIEIHQAEDIQVNPIE | | | |
| | | GKDKRAIDKGFSEVITSSSGRKYGE | | | |
| | | QFNQLLKKESDRLSEKNKKRNKIRA | | | |
| | | LTDKYEKKGDIVKSEIIKKNNLGKR | | | |
| | | KYFYQKEINLNEIKQFINLSLNRFITE | | | |
| | | ERPAVMVTEDLRFTNWNKKLSKNV | | | |
| | | KRYFSSWLKGYLQERIDYKVMLNG | | | |
| | | VQQVVVNSAYGSQICHLCGRFGVR | | | |
| | | NGDKFYCEIHGVLDADHNAALNYL | | | |
| | | ARMSDPDITIYTPYRKVKDILQERLR | | | |
| | | LSNQDSRYSVIKTGQWESERTDYV | | | |
| Resolvase | GAQ93499.1 | MFLGFKAAKAHFGVSSCTLRRWAN | 1506 | Resolvase, N | Klebsormidium |
| | | EAKVVSKRTAGNHRVFLAATSQAEE | | terminal | nitens |
| | | GRERLAYCRVSSSKQRDDLQRQKDF | | domain | |
| | | LAHQYPGHRIVTDVGSGINFKRPGL | | | |
| | | LSLLERVLQGRVSQVVVTSKDRLCR | | | |
| | | FAFELLQWICLRQGTQILVLDSGDKS | | | |
| | | PNEEKIRAFPTAQQRNILRRLVGGCR | | | |
| | | KLYNEAVSMIRDRRLPFASEAEFREA | | | |
| | | ERERGLRLAERKRKARSGQEEESGR | | | |
| | | GEGAGAYKGTKHPWLDKVYVKNF | | | |
| | | LVPEKSAFVRANPYLKDVPKETRQQ | | | |
| | | AVEDAIEACKAALSNVRAGNIEHFS | | | |
| | | LGFRKKKDPRWSLAVAFNAVSGSRF | | | |
| | | WPRKIKEFGQLRVAEPGHLRPHYDR | | | |
| | | ELKVSKDELGRYWMLVLSAKGPAR | | | |
| | | GTNEAAARVEALAESTRENQAGDR | | | |
| | | PVAAIDPGVRTRHSIYMTDGRVVDV | | | |
| | | GGGDIARVVRLCRHVDRCISALKKG | | | |
| | | EFCVSRKHVRREPGASTMRLFDHYR | | | |
| | | PAKKEQGAHVVPLDGRSREHIRAK | | | |
| | | MHRLRAKVQALKDEVDNQTVAYLV | | | |
| | | RECKAVLLPPFDTHRMATRLNHKTA | | | |
| | | RAMMQWRHGAFKAKLLERAARMG | | | |
| | | VQVMVVPEAYTSKTCGACGWLHPS | | | |
| | | LGGAKTFQCRSCGVELDRDHNGAR | | | |
| | | NIFLRAIRSWGVDSPE | | | |
| Resolvase | RAO55469.1 | MLIRVLGAGVNLKEWAAATGVSYA | 1507 | Resolvase, N | Micromonospora |
| | | TARRRYEAGALPVPTYRLGRLMVG | | terminal | saelicesensis |
| | | EPGTGTLAEVGRVVVYARVSSADQK | | domain | |
| | | TDLDRQVARVTVWATGQRLAVDSV | | | |
| | | VTEVGSALNGHREKFLALLRDPAVT | | | |
| | | TIVVEHRYRFARFGAEYVEAALSAQ | | | |
| | | RRRLLVVDPAEVDDDLVRDPHLAVR | | | |
| | | TPIRSPCRGEPSSERGRSGYRGTVVK | | | |
| | | TLQAYRFALDLSPRQERAVLAHAGA | | | |
| | | ARVAHNWALARVKAVMSQRAAERT | | | |
| | | YGVPDELLSPPISWSLPSLRKAWNA | | | |
| | | AKDEVAPWWAECSKEAFNTGLDAL | | | |
| | | ARALKNWSDSRKGARKGRAVGFPR | | | |
| | | FKSRRRSTPTVRFTTGVMRIEADRG | | | |
| | | HVVLPRLGALRLHESARKLARRLEA | | | |
| | | GTARIMSATVRREGGRWFVSFTCEV | | | |
| | | ERAVRAPARPGSMVGVDLGVKHLA | | | |
| | | VLSTGERVANPRHLVVAARRMRRLA | | | |
| | | RAVSRCVTPDRRVRRVGSNRWARA | | | |
| | | QQELSRAHARVVGLRRDGLHQFTT | | | |
| | | RLTREHGTVVVEDLNVAGMLRNRR | | | |
| | | LARHVADASFAEVRRQLTYKSEWN | | | |
| | | GGRLVTAGRWYPSSKTCSGCGAVKT | | | |
| | | KLTLAERTYTCTACGLSLDRDLNAA | | | |
| | | LNLAALAREVTDDRSGRESLNGRG | | | |
| | | ADQKTRSRGQVAVKRLPGTARAGQ | | | |
| | | AGTVPPQGGTTAQRSLLSTRR | | | |
| Resolvase | RAO04710.1 | MLIRVLGAGVNLKEWAAATGVSYA | 1508 | Resolvase, N | Micromonospora |
| | | TARRRYEAGALPVPTYRLGRLMVG | | terminal | saelicesensis |
| | | EPGTGTLAEVGRVVVYARVSSADQK | | domain | |
| | | TDLDRQVARVTVWATGQRLAVDSV | | | |
| | | VTEVGSALNGHREKFLALLRDPAVT | | | |
| | | TIVVEHRYRFARFGAEYVEAALSAQ | | | |
| | | RRRLLVVDPAEVDDDLVRDPHLAVR | | | |
| | | TPIRSPCRGEPSSARGRSGYRGTVVK | | | |
| | | TLQAYRFALDLSPRQERAVLAHAGA | | | |
| | | ARVAHNWALARVKAVMSQRAAERT | | | |
| | | YGVPDELLSPPISWSLPSLRKAWNA | | | |
| | | AKDEVAPWWAECSKEAFNTGLDAL | | | |
| | | ARALKNWSDSRKGARKGRAVGFPR | | | |
| | | FKSRRRSTPTVRFTTGVMRIEADRG | | | |
| | | HVVLPRLGALRLHESARKLARRLEA | | | |
| | | GTARIMSATVRREGGRWFVSFTCEV | | | |
| | | ERAVRAPARPGSMVGVDLGVKHLA | | | |
| | | VLSTGERVANPRHLVVAARRMRRLA | | | |
| | | RAVSRCVTPDRRVRRVGSNRWARA | | | |
| | | QQELSRAHARVVGLRRDGLHQFTT | | | |
| | | RLTREHGTVVVEDLNVAGMLRNRR | | | |
| | | LARHVADASFAEVRRQLTYKSEWN | | | |
| | | GGRLVTAGRWYPSSKTCSGCGAVKT | | | |
| | | KLTLAERTYTCTACGLSLDRDLNAA | | | |
| | | LNLAALAREVTDDRSGRESLNGRG | | | |
| | | ADQKTRSRGQVAVKRLPGTARAGQ | | | |
| | | AGTVPPQGGTTAQRSLLSTRR | | | |
| Resolvase | COW23552.1 | MNLAVWAERNGVARVTAYRWFHAG | 1509 | Resolvase, N | Mycobacterium |
| | | LLPVPARKAGRLILVDDQPADRSRR | | terminal | tuberculosis |
| | | ARTAVYARVSSADQKPDLDRQVARV | | domain | |
| | | TAWATTEQIAVDKVVTEVGSALNGH | | | |
| | | RRKFLALLRDPSVKRIVVEHRDRFC | | | |
| | | RFGSEYVEAALAAQGRELVVVDSA | | | |
| | | EVDDDLVRDMTEILTSMCARLYGKR | | | |
| | | AAQNRAKRALAAXXXXXXGARRR | | | |
| | | RVGGCLMAKFEIPEGWMVQAFRFT | | | |
| | | LDPTAEQARALARHFGARRKAYNW | | | |
| | | TVATLKADIDAWQATGIQTAKPSLRV | | | |
| | | LRKRWNTVKNDVCVNIETGVVWW | | | |
| | | PECSKEAYADGIDGAVDAYWNWQN | | | |
| | | SRSGKRDGKRMGFPRFKKKGRDPD | | | |
| | | RVTFTTGAMRVEPDRSHLTLPVIGTV | | | |
| | | RTHENTRRVERLIAKGRSRVLAITVR | | | |
| | | RNGTRIDASVRVLVQRPQQPKVTDP | | | |
| | | GSRVGVDVGVRRLATVATADGAVLE | | | |
| | | RVPNPRPLDAALNELRHVCRARSRC | | | |
| | | TKGSRRYRERTTEISRLHRRVNDVRT | | | |
| | | HHLHCLTTHLAKTHGRIVVEGLDAA | | | |
| | | GMLRQQGLSGARARRRGLSDAALG | | | |
| | | TPRRHLSYKTGWYGSQLVVADRWF | | | |
| | | PSSKTCHVCGHVQEIGWAEHWQCD | | | |
| | | SCSASHQRDDCAAINLARYEDTSSV | | | |
| | | VGPVGAAVKRGADRKTRPGRAGGR | | | |
| | | EARKGSSRKAAEQPRDGVQVA | | | |
| Resolvase | XP_004367500.1 | MQGPLADLPTNSTSRPWLVGPRNTT | 1510 | Resolvase, N | Acanthamoeba |
| | | KLNHRTRQILESGEDAFVPSNEVTK | | terminal | castellanii |
| | | YYNVTAACLRQWANKGEVRVLRIG | | domain | str. Neff |
| | | ELGKRLYNAKDLKSKLVGRDGGAQ | | | |
| | | QQRQQQRKRFAYAQVSSEHQRGDL | | | |
| | | ERQVGELRRLCPNHKIVTNVASGLN | | | |
| | | WKRKGVRAILDQCLEGMVEEVAVL | | | |
| | | HRDRLARFGVELMEYLFAKNDVRL | | | |
| | | VVVGEGATADAETHAVLEAVVDPA | | | |
| | | QELADDLIAQPWRKQQAANLKTAQ | | | |
| | | KKANRKKASAAKTKVMEASGDGD | | | |
| | | TNQPGGKRKRKGKERAPISEEGEEE | | | |
| | | EEPAPYDGKCRKIRVFPNRWQKDLL | | | |
| | | KSWMGTVRWTFNACNAAVRAGLS | | | |
| | | GHSEAELRSRFVDNEAFGKPHLPGP | | | |
| | | STLWVLDTPRDIRDQAAKELSAAYK | | | |
| | | NGTKAHGKGKFEVKFKSPKKMAQQ | | | |
| | | CITSNARDWGRGRTSVFHGLFDSGR | | | |
| | | ALRASEPLPREMKHEFKIVRTRLGR | | | |
| | | YYLCVPMDLETRGESQAPSSSDDVG | | | |
| | | AECVFIDPGVRTFVTTFDLSGRIHEF | | | |
| | | GTGSIGRIEKLCRRLDDLISRTYAKK | | | |
| | | PDDRQRFLRGKKKRWRMRRAALR | | | |
| | | MRRRIRDLIDEAHRKIALWLCENHR | | | |
| | | VILWPLSGVSNMVVAKEDLKQRKR | | | |
| | | RIGAKSVRAMLTWSWYRFQEWLKH | | | |
| | | KVREFPWCRLVLTSEAHTTKTCTHC | | | |
| | | GTPNHDVGRSEVFRCADAACPNRS | | | |
| | | AHRDHHGARNNGLRFLTEWANLPT | | | |
| | | TTTTTTMTQRLPTRVIDLTTSGLND | | | |
| Resolvase | XP_004367500.1 | MQGPLADLPTNSTSRPWLVGPRNTT | 1511 | Resolvase, N | Acanthamoeba |
| | | KLNHRTRQILESGEDAFVPSNEVTK | | terminal | castellanii |
| | | YYNVTAACLRQWANKGEVRVLRIG | | domain | str. Neff |
| | | ELGKRLYNAKDLKSKLVGRDGGAQ | | | |
| | | QQRQQQRKRFAYAQVSSEHQRGDL | | | |
| | | ERQVGELRRLCPNHKIVTNVASGLN | | | |
| | | WKRKGVRAILDQCLEGMVEEVAVL | | | |
| | | HRDRLARFGVELMEYLFAKNDVRL | | | |
| | | VVVGEGATADAETHAVLEAVVDPA | | | |
| | | QELADDLIAQPWRKQQAANLKTAQ | | | |
| | | KKANRKKASAAKTKVMEASGDGD | | | |
| | | TNQPGGKRKRKGKERAPISEEGEEE | | | |
| | | EEPAPYDGKCRKIRVFPNRWQKDLL | | | |
| | | KSWMGTVRWTFNACNAAVRAGLS | | | |
| | | GHSEAELRSRFVDNEAFGKPHLPGP | | | |
| | | STLWVLDTPRDIRDQAAKELSAAYK | | | |
| | | NGTKAHGKGKFEVKFKSPKKMAQQ | | | |
| | | CITSNARDWGRGRTSVFHGLFDSGR | | | |
| | | ALRASEPLPREMKHEFKIVRTRLGR | | | |
| | | YYLCVPMDLETRGESQAPSSSDDVG | | | |
| | | AECVFIDPGVRTFVTTFDLSGRIHEF | | | |
| | | GTGSIGRIEKLCRRLDDLISRTYAKK | | | |
| | | PDDRQRFLRGKKKRWRMRRAALR | | | |
| | | MRRRIRDLIDEAHRKIALWLCENHR | | | |
| | | VILWPLSGVSNMVVAKEDLKQRKR | | | |
| | | RIGAKSVRAMLTWSWYRFQEWLKH | | | |
| | | KVREFPWCRLVLTSEAHTTKTCTHC | | | |
| | | GTPNHDVGRSEVFRCADAACPNRS | | | |
| | | AHRDHHGARNNGLRFLTEWANLPT | | | |
| | | TTTTTTMTQRLPTRVIDLTTSGLND | | | |
| Resolvase | XP_004344636.1 | MQGPLGETNERPWLHNPRNTAIVR | 1512 | Resolvase, N | Acanthamoeba |
| | | HRTRQLIEAGGDTYVPSSEATKYFG | | terminal | castellanii |
| | | VTAACLRKLADRGNLRTRRIGDKG | | domain | str. Neff |
| | | KRLYHCGDLLSQFPAVTRSEDGRTIQ | | | |
| | | TTTRPPRKRIAYARVSSEKQRPDLER | | | |
| | | QIAELRRLCPDHEIVSEVASGLNFRR | | | |
| | | KGLCAILDRCFAGLVDEVAVLHRDR | | | |
| | | LARFATELLEHVFKRHDVRLVVVGQ | | | |
| | | GDPAAAATLDALDPQRELADDLIAV | | | |
| | | TTFFVARQNGLRSAAHRRARRDRA | | | |
| | | ALEEGRGSTTSEPSEEESEERGGSEE | | | |
| | | SERDETDGSSSSSSSDDGGGRRKRQ | | | |
| | | RTSRRRRRRREAEAEGTAGAEGGG | | | |
| | | DGEGGMVAFDGKTRKICVFPTAQQ | | | |
| | | KTLLKRWIGTVRWTYNQCVAAVRG | | | |
| | | RQCAPTKKALRARFVTEFGLREAER | | | |
| | | IKRAKSGVGGGGDDDDVGISWVFE | | | |
| | | TPHDLRDQAVGQFVTAYKNAVQAH | | | |
| | | GRGKFDIAFRSAKRLQQETVVIRSR | | | |
| | | DWNRTRGEYAPIFGNTVLRSSESLPA | | | |
| | | KMDHEFRVMRTKLGRYYISIPVPLD | | | |
| | | VKHPIQQQQPDAVGGGDNQAPAVAP | | | |
| | | HAAAAIDPGVRTPFTVYSPDEARVY | | | |
| | | ELGANDFGRIRRLCHHLDDLVSRTT | | | |
| | | DRDVRKKRRRRMRRAAARMRRRIR | | | |
| | | DLVDDLHRRAAKWLCETFETIIYPH | | | |
| | | YETSNMVVGKSKRRGLHSKTVRAM | | | |
| | | LTWSHFRFKQHLLHKIREYPSGCRV | | | |
| | | VLVDESYTSKTCGGCGRINHGLGKS | | | |
| | | KLFWCEQCGFRTDRDWNGARNIWL | | | |
| | | KFLTEWCNGSSRGNDDDDKEQQQQ | | | |
| | | QQQ | | | |
| Resolvase | XP_004344636.1 | MQGPLGETNERPWLHNPRNTAIVR | 1513 | Resolvase, N | Acanthamoeba |
| | | HRTRQLIEAGGDTYVPSSEATKYFG | | terminal | castellanii |
| | | VTAACLRKLADRGNLRTRRIGDKG | | domain | str. Neff |
| | | KRLYHCGDLLSQFPAVTRSEDGRTIQ | | | |
| | | TTTRPPRKRIAYARVSSEKQRPDLER | | | |
| | | QIAELRRLCPDHEIVSEVASGLNFRR | | | |
| | | KGLCAILDRCFAGLVDEVAVLHRDR | | | |
| | | LARFATELLEHVFKRHDVRLVVVGQ | | | |
| | | GDPAAAATLDALDPQRELADDLIAV | | | |
| | | TTFFVARQNGLRSAAHRRARRDRA | | | |
| | | ALEEGRGSTTSEPSEEESEERGGSEE | | | |
| | | SERDETDGSSSSSSSDDGGGRRKRQ | | | |
| | | RTSRRRRRRREAEAEGTAGAEGGG | | | |
| | | DGEGGMVAFDGKTRKICVFPTAQQ | | | |
| | | KTLLKRWIGTVRWTYNQCVAAVRG | | | |
| | | RQCAPTKKALRARFVTEFGLREAER | | | |
| | | IKRAKSGVGGGGDDDDVGISWVFE | | | |
| | | TPHDLRDQAVGQFVTAYKNAVQAH | | | |
| | | GRGKFDIAFRSAKRLQQETVVIRSR | | | |
| | | DWNRTRGEYAPIFGNTVLRSSESLPA | | | |
| | | KMDHEFRVMRTKLGRYYISIPVPLD | | | |
| | | VKHPIQQQQPDAVGGGDNQAPAVAP | | | |
| | | HAAAAIDPGVRTPFTVYSPDEARVY | | | |
| | | ELGANDFGRIRRLCHHLDDLVSRTT | | | |
| | | DRDVRKKRRRRMRRAAARMRRRIR | | | |
| | | DLVDDLHRRAAKWLCETFETIIYPH | | | |
| | | YETSNMVVGKSKRRGLHSKTVRAM | | | |
| | | LTWSHFRFKQHLLHKIREYPSGCRV | | | |
| | | VLVDESYTSKTCGGCGRINHGLGKS | | | |
| | | KLFWCEQCGFRTDRDWNGARNIWL | | | |
| | | KFLTEWCNGSSRGNDDDDKEQQQQ | | | |
| | | QQQ | | | |
| Resolvase | RHZ49948.1 | MCIVIRDQLTFLNKNHLFNFLTKPPF | 1514 | Resolvase, N | Diversispora |
| | | YNLAQLNTTYQSAHKIQETYDVSVE | | terminal | epigaea |
| | | TLRRWADSGRIAIVRTPGGKRLYSIT | | domain | |
| | | DIQEIFRDNQQTQITQKAKICYARVS | | | |
| | | SEHQRDDLERQIANLRQYYPEYKIIS | | | |
| | | DIRLGLNWKRKGFVALLERIHTEGIE | | | |
| | | EVVVTRKDRLCRFGSELVEWIFEKN | | | |
| | | GTRLVVLGTDVSAESSEAGELAEDL | | | |
| | | LSIVTVFVARHNGMQTYDVSVETLR | | | |
| | | RWADSGRIAIVRTPGGKRLYSITDIQ | | | |
| | | EIFRDNQQTQITQKAKICYARVSSEH | | | |
| | | QRDDLERQIANLRQYYPEYKIISDIR | | | |
| | | LGLNWKRKGFVALLERIHTEGIEEV | | | |
| | | VVTRKDRLCRFGSELVEWIFEKNGT | | | |
| | | RLVVLGTDVSAESSEAGELAEDLLSI | | | |
| | | VTVFVARHNGMRSAANRRRRREVV | | | |
| | | KAQEEQELQNSSRQDTTYLSLSYAR | | | |
| | | GEVKTQTLDGNTVEKKGIEQTKKA | | | |
| | | LRAQCLNAANFNNTELQWVLETPY | | | |
| | | DIRDEAMNDLLKSYSSNFAAKRKKF | | | |
| | | KMKFCSKKNQQQSITILSKHWGKSK | | | |
| | | GVYTFLCKMKSAENLPAELHYDSRL | | | |
| | | VMNRLGEFYLCIPQPLEIWAENQDP | | | |
| | | TQSDAVIALDPAVEWGKNDISRIYQL | | | |
| | | SHIYDKIQSTHDSIHGKVHKRKRYK | | | |
| | | LRRVMLRIHKKICCLINDCYHKLAK | | | |
| | | WLCQSYRIILLSKFQTQGMVRREKW | | | |
| | | RIRSKTARMMLTWSHFRFRQYLLHK | | | |
| | | VREYPWCRVIICTEEYTSKTCGCCG | | | |
| | | HIHRKLGGSKVFRCPSCTAELDWDI | | | |
| | | NSARNILLRYLTITSKEPVYAGAGIY | | | |
| | | PLEPS | | | |
| Resolvase | RHZ49948.1 | MCIVIRDQLTFLNKNHLFNFLTKPPF | 1515 | Resolvase, N | Diversispora |
| | | YNLAQLNTTYQSAHKIQETYDVSVE | | terminal | epigaea |
| | | TLRRWADSGRIAIVRTPGGKRLYSIT | | domain | |
| | | DIQEIFRDNQQTQITQKAKICYARVS | | | |
| | | SEHQRDDLERQIANLRQYYPEYKIIS | | | |
| | | DIRLGLNWKRKGFVALLERIHTEGIE | | | |
| | | EVVVTRKDRLCRFGSELVEWIFEKN | | | |
| | | GTRLVVLGTDVSAESSEAGELAEDL | | | |
| | | LSIVTVFVARHNGMQTYDVSVETLR | | | |
| | | RWADSGRIAIVRTPGGKRLYSITDIQ | | | |
| | | EIFRDNQQTQITQKAKICYARVSSEH | | | |
| | | QRDDLERQIANLRQYYPEYKIISDIR | | | |
| | | LGLNWKRKGFVALLERIHTEGIEEV | | | |
| | | VVTRKDRLCRFGSELVEWIFEKNGT | | | |
| | | RLVVLGTDVSAESSEAGELAEDLLSI | | | |
| | | VTVFVARHNGMRSAANRRRRREVV | | | |
| | | KAQEEQELQNSSRQDTTYLSLSYAR | | | |
| | | GEVKTQTLDGNTVEKKGIEQTKKA | | | |
| | | LRAQCLNAANFNNTELQWVLETPY | | | |
| | | DIRDEAMNDLLKSYSSNFAAKRKKF | | | |
| | | KMKFCSKKNQQQSITILSKHWGKSK | | | |
| | | GVYTFLCKMKSAENLPAELHYDSRL | | | |
| | | VMNRLGEFYLCIPQPLEIWAENQDP | | | |
| | | TQSDAVIALDPAVEWGKNDISRIYQL | | | |
| | | SHIYDKIQSTHDSIHGKVHKRKRYK | | | |
| | | LRRVMLRIHKKICCLINDCYHKLAK | | | |
| | | WLCQSYRIILLSKFQTQGMVRREKW | | | |
| | | RIRSKTARMMLTWSHFRFRQYLLHK | | | |
| | | VREYPWCRVIICTEEYTSKTCGCCG | | | |
| | | HIHRKLGGSKVFRCPSCTAELDWDI | | | |
| | | NSARNILLRYLTITSKEPVYAGAGIY | | | |
| | | PLEPS | | | |
| Resolvase | GAQ92436.1 | MWWFYLKEEKRTKRPVGVVGSCD | 1516 | Resolvase, N | Klebsormidium |
| | | TERVVQPQESEKTPELQDIPPGHEKY | | terminal | nitens |
| | | ANVFEKHAERFDDAYDKIREETLCL | | domain | |
| | | AKRRRQYSIMLDASSSSFRRAKDHF | | | |
| | | GVSSSTPRRWANEGKIATKRTAGNH | | | |
| | | RVFSIPSLEEKETRACIAYCRVSSSKQ | | | |
| | | RDDLQRQRRFLSDQLPRHESVSDVG | | | |
| | | SGINFERPGLLSILERVLQGRVSEVV | | | |
| | | VASKDRLCRVHKSGGAIKEAEVALA | | | |
| | | FPSRCRKIRAFPTGHQRLILRKMVG | | | |
| | | GCRKLYNETVAMIRDRRLPFANVEA | | | |
| | | FEEAERRRKTRLEDRKRKKAEDDGS | | | |
| | | EFEEVHYKGTSHPWMDKVYVKNFL | | | |
| | | VPEDSDFMKANPYLKEIPKETRQQA | | | |
| | | VEDAIEAYKAAFSNMAVGNISNFEV | | | |
| | | GFRKKKDPRWSIAVAFNAVSGSRFW | | | |
| | | PRKVKDFGELVVAEPRHLRKRYGRE | | | |
| | | LKISKDQLGRYWMVIMNDKGPKAT | | | |
| | | TEEAAKGVEELRESTRENQAGDKPV | | | |
| | | AAIDPGIRTRHTIYMTDGRLVEVENQ | | | |
| | | DIQRIVRLCRHVDRCISALSKGELAV | | | |
| | | SKKHLKRNPKASVMALFDHYRPSK | | | |
| | | KVQGQHVVRLSGEDKNRIRQKMHQ | | | |
| | | LKAKIESLKNEIDDQTVAYLLRECKT | | | |
| | | VLLPPFDMHSMSTRLHHKTARAMM | | | |
| | | QWRHGTFKTKLVERAPGRGVRVMI | | | |
| | | VSEAYTSKTCGACGWLHPSLGHKV | | | |
| | | FCTADWTAAAPVFKPLQGDGMLRN | | | |
| | | LSLSGTSLCGADARPGVDNIFCSNDF | | | |
| | | ISKPLTQVQGSGNVTAQGKGRLCVV | | | |
| | | AKDQRVFCTDSIVQNPVWTKRGDL | | | |
| | | AIDLAMNESGGICALNPDGTIFCQPD | | | |
| | | LTSGKWVATNVAEKKNVNLATNIHG | | | |
| | | TKLCIFNSDKAPAYCHDNVFAGDKA | | | |
| | | GWYGLAAAGQRFGLEKV | | | |
| Resolvase | CAB1120549.1 | MAFVNTKTAIERLGVSNVTLRKWD | 1517 | Resolvase, N | Ectocarpus sp. |
| | | KLEAIPTIRTPGGQRVYDIETFQKTQ | | terminal | CCAP 1310/34 |
| | | ALRSEEARLCARRMAKDKKNASRI | | domain | |
| | | DIGYARVSFSKQKEDLGRQEQFIRDS | | | |
| | | CPGISILSDIGSGLNFKRKGFKKLLR | | | |
| | | CIMQGQIDRVLVAYKDRLCRFAFEII | | | |
| | | QFICDENNTELVVLNQNENGSAEAE | | | |
| | | LMEDLMAVVHVFSSRLYGKRSTGK | | | |
| | | RKRYTEGGAEDGALVETIREKLSDIE | | | |
| | | PHSTRAKKVRMYPTRKQKKILTKY | | | |
| | | MSDGRRVYNECVRKLIDGVSTSKIR | | | |
| | | DKAIRAPCMDKTMEKTLRTPEHVR | | | |
| | | QKAVDQFVAAQKGVETRETGSLHF | | | |
| | | RSKFNQKQRIGLQKKDSRIHGSSVH | | | |
| | | LTFKEFDENILLGEELVSDDGILTEIV | | | |
| | | RDRGVYYATVTKTRPVTPKSDGLRV | | | |
| | | VSLDMGSRKFGSFYSSDGSIGFIGEE | | | |
| | | AGEKLSRLIKKREYLKKLKESKSKL | | | |
| | | TKGWRKVSKRIANLVNEMHNKVAL | | | |
| | | YLVRNYDIIMVGRLSNGVMKTKSH | | | |
| | | RNQKLLHASLKHYQFRQRLINNGN | | | |
| | | DHGKKVAIVPEQLTTKLCDRCGFIN | | | |
| | | WKMKAEEVFTCPKCKHSCDRDIHS | | | |
| | | VDPGARPVAAIGGRNDVGGGGATG | | | |
| | | ERSGGAVDAPVSGKGVAKATQRKN | | | |
| | | KTKPKQVSPDESDGVRAVVAALRK | | | |
| | | AFAQAKLRNNDAKAAGKQEDDVH | | | |
| | | KYNGSFRACNLLRSRRSSTIGAGPNS | | | |
| | | GEICDAKVHSRPPRENVQVQRVLQG | | | |
| | | LQPPEVKKVFYDRCWAAINIRISGK | | | |
| | | NTRNDFSDLLDEIIDQSGFDTSLFPP | | | |
| | | KVPCRLQETVTREKEVSAKNSIVVH | | | |
| | | LEAKIKGFLRFRLMNDSQLAFQHLP | | | |
| | | AKDRSRIIVSLSNDCLEREMRGDLD | | | |
| | | PGYVPHVLQVRDPLAQVYATEPDVP | | | |
| | | VLKILKKKTHLFVELISMISNVAEEA | | | |
| | | SDNNRALREETKTRSDEEKVRKAFY | | | |
| | | MSERIKRGLVDRPKTCTVLPVWKLA | | | |
| | | PCFPHYSSSVVGSIFHKEGHDFSSVS | | | |
| | | RFISEHFDLRRVNRKGYKPSGFRSD | | | |
| | | GYQVQVTFMALVSKKPHVPGTTDL | | | |
| | | AKSGYQIAKKVVSLETQERGLFVLS | | | |
| | | QARKDSRKIVADRVHANNLTVVDP | | | |
| | | GCASVVSVRSCPLEFCRCCRERSRES | | | |
| | | TDWEMKGTEYSVKSGRTMLEGREK | | | |
| | | KRRSNDEYGRCFPRFSAVKKKTARK | | | |
| | | SSFLAYCRVAAETFKVMFAEKMKRA | | | |
| | | RKRSRFHSSRLVQKTVDKLASNIAA | | | |
| | | CPSDRKNIVLFGNGSFRAMKGHASA | | | |
| | | PRKKLVRAICSRVNVGMLDEFRTSK | | | |
| | | MCPGGCGGEMTDVQGGQRVRQCT | | | |
| | | TVCVGVENPCPLFENGVAFRCDRDA | | | |
| | | SATLNFCLAGYCGLVX | | | |
| Resolvase | CAG8449366.1 | MKTREFFTGDIYPNDNVYSMQIVED | 1518 | Resolvase, N | Cetraspora |
| | | HEDYYTKEYYMKIKCVATNSLDAYI | | terminal | pellucida |
| | | FLSGFPVYCEFKINNGFDDGIDEKIV | | domain | |
| | | KEILNYISLTNYKDYRSVKREDILGN | | | |
| | | EFYFLRVFFKNHNIRKKIIKKVRDCI | | | |
| | | KNKEDNYQIFDLYEDDISTPYSMFIA | | | |
| | | PRIKIKINGFEQGFPLSKTFKLNRTLS | | | |
| | | RKIYENKFHYSNLRFIDDIDTYISMF | | | |
| | | FDFETVDIENMKKGILGRVSIGCEID | | | |
| | | QAFMGVFLFFKGRDIIPFHTVAVLLQ | | | |
| | | NEEFPVEYERIDEKLDLIYVKNQKEF | | | |
| | | FLTKALLYYNFRPEKTGGWNNLGY | | | |
| | | DWKFTIRKLYELGIFEEFIQIATGKTK | | | |
| | | SIENIIKFDYRNEYNTVNANEKACH | | | |
| | | YFLKILGTEEYDQSTMFRQHYQTLS | | | |
| | | KWSLNTILGKLGLKLKLEFSIKEMY | | | |
| | | EILYNVYKGNFDEKTKKEKCTLILD | | | |
| | | YCIRDCIAPKEAIEFINKITEYRIISNL | | | |
| | | TYIPMYEYSYGNKTKMINNLIVYFA | | | |
| | | NREGFDISIKYDKKNVKEEYEGGLV | | | |
| | | KDPLKEFSVVSDGCLDFNSLYPSVM | | | |
| | | MQHNICFLTKLKKNEKEEGCHEISY | | | |
| | | KNSKGITKIIRFSKKRKGLIPTILELL | | | |
| | | VKRRKEAKKDRDRHEKSNPMYNY | | | |
| | | YNILQDTLKKIANSIYGQFGNQYSII | | | |
| | | CDYEVSASVTGAYARKYIKMASEY | | | |
| | | MQTKEISETNKEKKFIWKYTDTDSIF | | | |
| | | FRLSPILINEIIKRYENHLLDKNINEK | | | |
| | | DFIDVIYNIQKEMVFVTYNESMKLQ | | | |
| | | DEINEWMANENQAPRIIMEMEKIVS | | | |
| | | PNLYISKKKYNGLIFENSNYFFDDE | | | |
| | | WKDLEKHIKEEYKIDNVTKEIVNDF | | | |
| | | VKKNDEYKLLANGKKFSNLLAKGT | | | |
| | | DLVRRNSTLICRVILKKLLYTLFDYR | | | |
| | | EYGRYLYNMKFTKIEEYQDFINGVN | | | |
| | | KNDFAKETSKRVVEDFIKYLYSKET | | | |
| | | ELNLELFEQNARNNEEEEDMMTFTE | | | |
| | | TKDPLSITKYTMTSKYKPAEKIKKT | | | |
| | | YGVSTSTLRRWGDKGDVSCITMPG | | | |
| | | GKRMYSTEDIDNMFGRESKEKKKIC | | | |
| | | YARVSSEKQKEDLERQCNHLRSEYP | | | |
| | | EHEIITDIGSGLNWKRKGFTSLLERI | | | |
| | | YQGDIEEVVVTRKDRLCRFAYELVE | | | |
| | | WIFKKHEVKLMVLGTDVGSNEPET | | | |
| | | GELAEDLLSIVTVFTARHNGLRSAA | | | |
| | | NKRKRKEIENSKDTDILRQKCIKIEN | | | |
| | | FQNENKWVLETPYGIRDEALIDLLD | | | |
| | | AHKSNFKLKRQKFNIRYKKKKDKQ | | | |
| | | HSITIQARDWKRKRGEYAFLKNIRM | | | |
| | | SEELPNIEHAFNIILDKLGKFYICIPISI | | | |
| | | EEYYREDNEIISLDPGIRTFMTGYDPI | | | |
| | | DDCHMKLAKFLCDNYNTILLPKFET | | | |
| | | QEMVKRIKRKIRNKTARMMITWSH | | | |
| | | YRFRRFLEHKISEYPGRLLILCNEHY | | | |
| | | TSKTCGNCGYIKRNFGGSKIYKCDE | | | |
| | | CGFVIDRDYNENQTDWERSEKKDK | | | |
| | | EYTETILKQENDETTSMVKKFVRDM | | | |
| | | KMINVEVPKPRFNYYVIYKHGVKE | | | |
| | | VYKRMVLVDRFDPKKQRIDKYHYL | | | |
| | | KGIKSFLSVCIEETEKETDEWIKSIID | | | |
| | | KNTNKSIDEYSLKEDEQKGGKKRK | | | |
| | | KKEYDILIPKKQMKISSFFKKQNND | | | |
| | | NDDNDFFI | | | |
| Transposase_ | QIY66925.1 | MSGAEPAGSGKKKRRGFEARPGFH | 1519 | Transposase, | Streptomyces sp. |
| mut | | VVGHRLALDPSASALQALASHCGA | | Mutator | RPA4-2 |
| | | ARVAYNWAVRHVLASWSQRAAEET | | family | |
| | | YGVPEAERVAWRSWSLPSLRKAFNE | | | |
| | | AKHNDPFLREWWAQNSKEAYNTGL | | | |
| | | ANAAAAFDNYVKSRRGERKGARM | | | |
| | | GRPRFKPKRKTRPACKFTTGTIRLDD | | | |
| | | RRHIVLPRLGRIRLHEDVQPLVDAIA | | | |
| | | EGCRSNQGSKVHEQAVSSAQQASD | | | |
| | | VGRPLKGSAPIAEGGTRILSVTVRSE | | | |
| | | RGRWFAVLQTEERHTIAPAGRPGTA | | | |
| | | VGIDLGVKALLVMADSAGEVREVA | | | |
| | | NPKHYDQALTQLRKASRTVSRRRGP | | | |
| | | NRRTGQAPSRRWEKANAVPRSSSAE | | | |
| | | TSRTGTLCCVLWESAWAEFVPFLSF | | | |
| | | DVEIRKVICSTNAIESVNARIRKAVL | | | |
| | | EFQWLSQHRSTTMVPSRPARRAADT | | | |
| | | QRQPSSGATWVRMLSSTWAL | | | |
| Y1_Tnp | SFU97206.1 | MEADPTLAVAEIVNRFKSRSSRLMR | 1520 | Transposase | Methylobacterium |
| | | QEFPALRSRLPTLWSRSYYPGSVGH | | IS200 like | sp. UNCCL125 |
| | | VSAKVVEAYIAAQKGTGAVAVRSYK | | | |
| | | YRLRPNRAQTAALDAMLRDFCGLY | | | |
| | | NACLQQRVEAYRRRGLNLRYGPQA | | | |
| | | SELKACRACDPDGLGRWSFSALQQ | | | |
| | | VLRRLDQTYAAFFKRGHGFPRFRAS | | | |
| | | ARYHAATFRIGDGLTVKKDRRIGVV | | | |
| | | GVVGVPGGLKVAWHRDLPDEAKLG | | | |
| | | TAILTRQQGKWFMVLSVEAEFAETC | | | |
| | | GTGTVGIDLGLNSLIATSDGETVEMP | | | |
| | | RFARKAQKAQRWRQRALARCKRGS | | | |
| | | KRRLKAKARLAAGSAKIARQRRDH | | | |
| | | LHKLSRSLVSRYWGIAFEDLTMTGL | | | |
| | | NLSRVWDSQSQDTRDPNPRLQMWR | | | |
| | | GSGPGCCCGDRRASAGFRAGTRPSG | | | |
| | | HKPAGCRIAVPRSRLLQRAELSRLGS | | | |
| Y1_Tnp | MBV8270253.1 | MEYRRDEHRIHLVVYHLIWCPRRRK | 1521 | Transposase | Planctomycetaceae |
| | | PVLMGDVARDCRSLIEAKCAEHGW | | IS200 like | bacterium |
| | | TIETLAIRPDHVHLFVRAWPKDSAA | | | |
| | | DVLKAVKGVTAHALRKKYPHLRKT | | | |
| | | PSLWTRSYFASTAGNVSQETIRRSIE | | | |
| | | AQKGLEMIRTHILPCTIPRARADELN | | | |
| | | RASGAIYTGILVAHWRLVRKKGLWL | | | |
| | | SEASGTRWSDTRTDARMHAHSIDA | | | |
| | | AQQGFYKACETARGLRQAGIAEAK | | | |
| | | FPYHRKKFRTTIWKATGIKREGNILR | | | |
| | | LSGGGRTKKERDERAVEVPIPEQLR | | | |
| | | DCLKFLEVRLVYDKYARRYTWHVV | | | |
| | | VENGLKPKPAPGRNVVSVDLGEIHP | | | |
| | | AVVGDRTHAIVITCRERRSRSQGHA | | | |
| | | KRLATISRAIARKAKTSRRRRRLIRA | | | |
| | | KVRMKAKHARILRDIEHKVSHEVV | | | |
| | | AFAAERQAGTIVIGDIRDIADGIACG | | | |
| | | T | | | |
| Y1_Tnp | MCL9760917.1 | MSGWAGGVYDLGYHVVWCPKYRR | 1522 | Transposase | Frankia sp. AiPa1 |
| | | AVLVGQVRDRLDTLIQQKCAEHGW | | IS200 like | |
| | | PIVALEIEPDQVHNAALQERRDAYA | | | |
| | | HPSKTKVRYGDQSAQLKEIRAYDPD | | | |
| | | LARWSFSSQQATLRRLNLAFEAFFR | | | |
| | | RVKAGETPGYPRFKGAGWFDTVTW | | | |
| | | PVDNDGCRWDSQPEHPTRTFVRLQ | | | |
| | | GVRHVRVHQHRPVRGRVKTLSVKR | | | |
| | | ESARWYLILSCDDVPSKPLPPTGAVV | | | |
| | | GIDLGVASLVTDSNGEHYGNPRFLR | | | |
| | | RSADRLADAQRDLSRKRRGSKRRR | | | |
| | | MAVQRVAVLSRQVARQRVDLANKT | | | |
| | | VNEIVADHDLIVVEKLNIKGMVKRA | | | |
| | | RPRPDPDSAGGFLPNGQAAKSGLNK | | | |
| | | SIHDAGWGVFLNVLRAKAESAGRL | | | |
| | | VVEVNPRHTSQRCPGCGHVAAENR | | | |
| | | LTQATFLCVRCGHAAHADVNAAVNI | | | |
| | | LRAGLALQAATPSS | | | |
| Y1_Tnp | EDT84099.1 | MRALKGVSARLLMKEYGDELKKKL | 1523 | Transposase | Clostridium |
| | | WGGHLWNPSYFIATVSENTEEQIRDI | | IS200 like | botulinum Bf |
| | | FKVKNKNRRVVDLFMKIIIKGFKYRI | | | |
| | | YPTKEQEIQLNKTFGCVRFVYNQIL | | | |
| | | AKKIDLYKNESKSISKTICNNYCNRE | | | |
| | | LKKEYPWLKEVDKFALTNSIYNLDS | | | |
| | | AYQKFFKEHTGFPKFKSKKNHYYSY | | | |
| | | TTNFTNNNIKVDFENNKIQLPKLKW | | | |
| | | IKAKLHKEFQGRILFATVSKTPSNKY | | | |
| | | FVSLNIECEHQELKQNNNKIAMDLG | | | |
| | | IKDLLITSNGTKIDNKKLSYKYEQKL | | | |
| | | AKLQRQIAKKKIGSNNWRKQRIKIA | | | |
| | | RLHEKIANIRKDNLHKISHKIVKENQ | | | |
| | | LIFSENLNIKGMVKNHNLAKSIHDC | | | |
| | | GWYELTRQLTYKSEWNNRIYHKVD | | | |
| | | RFHPSSQLCNVCGYKNEDTKNLNIR | | | |
| | | FWECLQCHTKHDRDENASINILNQG | | | |
| | | LKELKLEKVS | | | |
| Y1_Tnp | TCS68577.1 | MTEVMFTFYSRLIPIQKYPKFINAYK | 1524 | Transposase | Effusibacillus |
| | | SASSRLVKKEFPVIRKSLWKEYFWS | | IS200 like | lacus |
| | | RSYCLLSTEVPPLKLSKSTLKHKVN | | | |
| | | ESDFRMDSVLHFEIRPTAEQEKQLFH | | | |
| | | TFDLCRKLYNYALDQRIRSYKETGK | | | |
| | | GLTYRDQQNMLPAFKEANPEYKAV | | | |
| | | QSQVLQDVLRRLDRAFVNFFEKRA | | | |
| | | GYPRFKDKLRFRSITIPQSDVRRNFG | | | |
| | | KEGYIYIPKIGHIKLNAHQAFDPSKV | | | |
| | | KIINVKFQNGKWYTNLTVETDSKPP | | | |
| | | VSDIQLAVGIDMGLFQIAVTSDGEQY | | | |
| | | ENPRWITKSEKRLKKLQRRLSQKHK | | | |
| | | GSQNRQKAKHQLQKLHDHISNQRK | | | |
| | | DYLHKISHRLVQKYVLICIEDLQVK | | | |
| | | GMMKNHRLAKSIANASWNRLANY | | | |
| | | LEYKSRRFGKTLVKVNPKNTSQKCS | | | |
| | | NCRQIVKKNLSERIHQCPYCHVVLD | | | |
| | | RDLNAAINILQAGLNMIA | | | |
| Y1_Tnp | MBW8381061.1 | MYHVVWCSKYRRTVLTDQVELRLK | 1525 | Transposase | Youngiibacter sp. |
| | | EIIASVCKEKEVELFEMEVMPEHIHL | | IS200 like | |
| | | LLEVDPQFGIHRVVKALKGQSSREL | | | |
| | | REEQNMQLTVQMKLIPDNEQKALIE | | | |
| | | YILNSYIATVNDIVADFVSMGQIDKR | | | |
| | | TSADISTSMPSALKNQAIQDAMSVF | | | |
| | | KKYTKDLKSAIRFNESNPSKKHKTV | | | |
| | | IVPVLKKPVAIWNNQNYSIGEDVISF | | | |
| | | PVWKDGKSKKLSFRIVATEYQKTLL | | | |
| | | QNKLGTLRITRKSGKYIAQIAVDIPC | | | |
| | | ESYHGSSMMGIDLGLKVPAVAVTDT | | | |
| | | GKIQFFGNGRENKYKKRMARAKRK | | | |
| | | ALGKAKKIKALKKLDNKEQRWMK | | | |
| | | DKDHKLSKEIVNFAKTNKVKTIQLE | | | |
| | | ELAGIRQTARTSRKNAKNLHTWSFY | | | |
| | | RLAQFIQYKANLVGIEVVYVNPKYT | | | |
| | | SQTCPVCGTKNHANDRKYKCPCGF | | | |
| | | KTHRDILGAMNIITAPVIDGNSLSA | | | |
| Y1_Tnp | MBA9002912.1 | MVRVKLVKPIKGRSSRVLREEFPHL | 1526 | Transposase | Thermomonospora |
| | | KSQLPTLWTNSSFVATVGGAPLSEV | | IS200 like | cellulosilytica |
| | | KRYVEQQKSWQMLTGRRYLLAFTP | | | |
| | | GQETFAELVGDACRMVWNTGLEQR | | | |
| | | RAYRRRGAFIGYVEQARQMAEAKK | | | |
| | | DFPWLAEAPSHTLQQTLRDLEKACK | | | |
| | | THGTFKVRWRSKRKNAPSFRFPDPK | | | |
| | | HIAVERVSRRWGRVRLPKLGWVRFR | | | |
| | | WTRPLGGMVRNVTVLKDGGRWYIS | | | |
| | | FCVEDGLAESTPNGKPPVGVDRGVA | | | |
| | | VAVATSDGWMRDREFVTLGEAVRL | | | |
| | | KRLQQQLARQRKGSARRSATKAKIG | | | |
| | | RLNARVRARRTDFVAWTANRLTRD | | | |
| | | HGLVVVEDLKVKNMTASAKGTLEQ | | | |
| | | PGSRVRQKAGLNRSILAKGWGGLL | | | |
| | | AALEHKARCNGSRIVRVPPAYTSQT | | | |
| | | CAACGHCAPDNRESQAVFRCRACG | | | |
| | | HQANADVNAAKNILAAGLAVTGRG | | | |
| | | DLAAGRSAKRQPPETEAV | | | |
| Y1_Tnp | SCI79596.1 | MKLDNNAHSVFSLNYHLVLVVKYR | 1527 | Transposase | uncultured |
| | | RQIFNDDISDRAKEIFEYIAPNYNIIL | | IS200 like | Eubacterium sp. |
| | | EEWNHDKDHVQILFRAHPNTEISKFI | | | |
| | | NAYKSASSRLLKKEFPQIRKKLWKE | | | |
| | | HFWSQSFCLLAKTFGCVRMVYNHW | | | |
| | | LDRKIRQYEENKTNVTYTACAKEM | | | |
| | | AEMKKTEEYAFLREVDSISLQQSLR | | | |
| | | HLDTAFQNFFKQPKTGFPRFKSKKR | | | |
| | | NKNSYSTVCINSNITISNGYLKLPKIG | | | |
| | | QVRLKQHRDVPKEYRLRSVTVSQTS | | | |
| | | SGKYYASILFEYEDQVQEKEIETFLG | | | |
| | | LDFSMHGLYRDSNGNEPAYPRYYRK | | | |
| | | AEKKLAREQRRLSKMQKGSNNRRK | | | |
| | | QRMKVAKLHEKVCNQRKDFLHKQS | | | |
| | | RQIANAYDCVCVEDLDMKAMSQSL | | | |
| | | KFGKSVSDNGWGMFTTFLKYKLKE | | | |
| | | QGKKLVKVDRFFASSQICSACGYKN | | | |
| | | MKTKDLALRQWDCPQCGTHHDRDI | | | |
| | | NAAINIRNEGMRLVMA | | | |
| Y1_Tnp | WP_138373607.1 | MDKNYIFAEHTNVTHGSGYVYLLQ | 1528 | Transposase | Drancourtella sp. |
| | | YHIVWVTKYRKPVLVGMVAAETKR | | IS200 like | BSD2780061688b_ |
| | | HLLETMEQLQMECLAMEVMPDHIH | | | 171218_E11 |
| | | LLVMELIPDDSQRAGFAQQIGNARF | | | |
| | | MRNQYLNDRIAYYKETRKTLPVDV | | | |
| | | YKKKYFPKLKEQYSFLTLSDKFALE | | | |
| | | SAIEHVDTAYKNFFEGRAAFPKFASK | | | |
| | | WKPSGNTYTTKWTGNNIRLEEHDG | | | |
| | | LPYIKLPKVGLVRFILPKKQTIQTLVP | | | |
| | | HGTSILSVAVKKKGDRYTASLQLET | | | |
| | | VVESPVQLNQMSVRDIMAADMGIK | | | |
| | | LFAIIGGEDWEKEIPNPRWIRIHEKRL | | | |
| | | RRLQKSLSRKKYDKETHTGSKNWE | | | |
| | | KAKQKVAAEHRKIANQRKDFQHKL | | | |
| | | SRRIVDRCSVFCCEDLNIRGMVKNR | | | |
| | | RLAKEISSASWGQFLTMVKYKMER | | | |
| | | QGKHFIQVSRWFPSSQTCSRCGFQN | | | |
| | | TVVKDLVIRSWKCPKCGTYHNRDV | | | |
| | | NAKNNILAEGIRLLQKNGIIVTV | | | |
| Y1_Tnp | WP_138373607.1 | MDKNYIFAEHTNVTHGSGYVYLLQ | 1529 | Transposase | Drancourtella sp. |
| | | YHIVWVTKYRKPVLVGMVAAETKR | | IS200 like | BSD2780061688b_ |
| | | HLLETMEQLQMECLAMEVMPDHIH | | | 171218_E11 |
| | | LLVMELIPDDSQRAGFAQQIGNARF | | | |
| | | MRNQYLNDRIAYYKETRKTLPVDV | | | |
| | | YKKKYFPKLKEQYSFLTLSDKFALE | | | |
| | | SAIEHVDTAYKNFFEGRAAFPKFASK | | | |
| | | WKPSGNTYTTKWTGNNIRLEEHDG | | | |
| | | LPYIKLPKVGLVRFILPKKQTIQTLVP | | | |
| | | HGTSILSVAVKKKGDRYTASLQLET | | | |
| | | VVESPVQLNQMSVRDIMAADMGIK | | | |
| | | LFAIIGGEDWEKEIPNPRWIRIHEKRL | | | |
| | | RRLQKSLSRKKYDKETHTGSKNWE | | | |
| | | KAKQKVAAEHRKIANQRKDFQHKL | | | |
| | | SRRIVDRCSVFCCEDLNIRGMVKNR | | | |
| | | RLAKEISSASWGQFLTMVKYKMER | | | |
| | | QGKHFIQVSRWFPSSQTCSRCGFQN | | | |
| | | TVVKDLVIRSWKCPKCGTYHNRDV | | | |
| | | NAKNNILAEGIRLLQKNGIIVTV | | | |
| Y1_Tnp | TLY96581.1 | MVDLVTNRNCLYQTAYHVIWCPQY | 1530 | Transposase | Gammaproteobacteria |
| | | RRAALTGPIAAEVGTLLEAICGERG | | IS200 like | bacterium |
| | | WPVISKEIQPDHIHLVVSIPPAIAVAN | | | |
| | | AVKVLKGVSARHLLQRFPALKKRL | | | |
| | | WGGHLWSPSYYVGTAGTLSAADAC | | | |
| | | NRLVPLVQAARCWNRVALHQLGYR | | | |
| | | ALRQQTSLGAQMVCNAIFSVCKAY | | | |
| | | RSQGALGRIPQDTPVPPLSFHRTSVH | | | |
| | | FDHRTYTLKDETVSLNTLQGRMRVP | | | |
| | | MILGDHQRKILTSGLPKEAELVFRAG | | | |
| | | QWFFNLAVESADGERVASGPVMGV | | | |
| | | DVGENTLAATSTGRVWGGETLRHR | | | |
| | | RDQHLALRRRLQSNGSQSATQRLRQ | | | |
| | | VSGKERRRVRHVNHETSKAILEEAR | | | |
| | | RIGAATIVMEDLTHIRDRLRAGRRM | | | |
| | | RARLHRWAFRQLQGFLEYKARAIGI | | | |
| | | SVVYVNPAYSSQTCSACGQLGTRRK | | | |
| | | HRFECSCGLRAHADLNASRNLARIG | | | |
| | | ETAVSPRAVVNTPDVGCVACHASP | | | |
| Y1_Tnp | EEM92921.1 | MMNDYRRTKTTISLINYHFVFYPRYI | 1531 | Transposase | Bacillus |
| | | RKIFLNTKVEERFKELVQEICNELDI | | IS200 like | thuringiensis IBL |
| | | VIVAMECDTDHVHLFLNTLPTLSPA | | | 200 |
| | | DTMAKIKGVTSKKLREEFPHLQHLP | | | |
| | | SLWTRSYFVSTAGNVSSETIKHYVES | | | |
| | | QKTRGVKFVTQTITVKAKLLPTKEQ | | | |
| | | IRLLKQSSSDYIKLINTLVFEMVESK | | | |
| | | QSTKKSTKDIEANLPSAVKNQAIKD | | | |
| | | AKSVFSTKVKKNKYKIVPILKRSVC | | | |
| | | VWNNQNYSFDYTHISIPFMVNGKPT | | | |
| | | RLKVRTLLIDKHNRNFDLLKHKLGT | | | |
| | | LRITKKSGKWIAQISVTVPTIEKTGT | | | |
| | | KILGVDLGLKVPAVAITDDDKVRFF | | | |
| | | GNGRKNKYMKRKFRSVRKKLGKA | | | |
| | | KKLNALRQLDDKEQRWMQDQDHK | | | |
| | | VSREIVDFVTNNTISVIRLKQLTNIRQ | | | |
| | | TARTSRKNEKNLHTWSFFRLAQFIE | | | |
| | | YKAKLVGIKVEYVTPSYTSQTCPKC | | | |
| | | TKKNKAQDRKYKCQCGFEKHRDIV | | | |
| | | GAMNIRYATVVDGNSQSA | | | |
| Y1_Tnp | EEM92921.1 | MMNDYRRTKTTISLINYHFVFYPRYI | 1532 | Transposase | Bacillus |
| | | RKIFLNTKVEERFKELVQEICNELDI | | IS200 like | thuringiensis IBL |
| | | VIVAMECDTDHVHLFLNTLPTLSPA | | | 200 |
| | | DTMAKIKGVTSKKLREEFPHLQHLP | | | |
| | | SLWTRSYFVSTAGNVSSETIKHYVES | | | |
| | | QKTRGVKFVTQTITVKAKLLPTKEQ | | | |
| | | IRLLKQSSSDYIKLINTLVFEMVESK | | | |
| | | QSTKKSTKDIEANLPSAVKNQAIKD | | | |
| | | AKSVFSTKVKKNKYKIVPILKRSVC | | | |
| | | VWNNQNYSFDYTHISIPFMVNGKPT | | | |
| | | RLKVRTLLIDKHNRNFDLLKHKLGT | | | |
| | | LRITKKSGKWIAQISVTVPTIEKTGT | | | |
| | | KILGVDLGLKVPAVAITDDDKVRFF | | | |
| | | GNGRKNKYMKRKFRSVRKKLGKA | | | |
| | | KKLNALRQLDDKEQRWMQDQDHK | | | |
| | | VSREIVDFVTNNTISVIRLKQLTNIRQ | | | |
| | | TARTSRKNEKNLHTWSFFRLAQFIE | | | |
| | | YKAKLVGIKVEYVTPSYTSQTCPKC | | | |
| | | TKKNKAQDRKYKCQCGFEKHRDIV | | | |
| | | GAMNIRYATVVDGNSQSA | | | |
| Y1_Tnp | ACV62680.1 | MERKTNHCVYNINYHIVFCPKYRRK | 1533 | Transposase | Desulfofarcimen |
| | | AITGKVEDAVKQIIQEICNTYGYLRA | | IS200 like | acetoxidans DSM |
| | | LPPTEYLSLPWLKKKYFWGSGLWS | | | 771 |
| | | RGYYIGTAGNVSTETFANILKPRNIP | | | |
| | | GGGEIVSKCKNNQSKKSKGIDILVN | | | |
| | | KFPVYLTPEQTSLARTLQREAAKVW | | | |
| | | NTTCIVHRTIYIKHHCWLDEGAMKA | | | |
| | | FVKGKYGVHSQSAQAIVETYFECCE | | | |
| | | RTGKLREQGVTDWRYPHRRKRFFT | | | |
| | | VTWKPLGINYEGKMLTLSNGRGRES | | | |
| | | LILNLPKRLSGAVIKLVQLVWHRNLY | | | |
| | | WLHVTVEKPALKKVQGGVTAAIDP | | | |
| | | GEVHAVAITDGKKSLVVSGRLLRSLS | | | |
| | | RLRNKVLRRLQKAISKTKKGSKQH | | | |
| | | NKLLAAKYRFLNNIERRIEHVIHTIS | | | |
| | | AIVSKWCFEHNVNTVYIGNPEGVRK | | | |
| | | KDCGKKHNQRMSQWTFGELRRML | | | |
| | | EYKLKRHGIKLISVDERGTSGTCPAC | | | |
| | | AEYTKQTGRIYKCGNCGFAGPHRD | | | |
| | | MVGASGILDKSVNGKFTKGRKLPE | | | |
| | | KVEYARLKVKTA | | | |
| Y1_Tnp | MBV8268687.1 | MGSRRDEHRIHLVVDHLLGCPKRR | 1534 | Transposase | Planctomycetaceae |
| | | KPVLVGDVARDCRSLIEAECHDHG | | IS200 like | bacterium |
| | | WTIEDLAIQPDHVHLFIRVWPMDSA | | | |
| | | ADVLKAVQGVPAHAPRKRYPHLRK | | | |
| | | TPSLWTRSSFASTAGNVSQETIRRSIG | | | |
| | | AQKGMEMFKAFVFRLYPSASHRRR | | | |
| | | LEAVRETCRRFYNTLLRQRKDASEL | | | |
| | | RGVSITKTEQLRLVKVEKDTSPYAS | | | |
| | | GIHSPILQAVVADLDKAFQAFFRRVQ | | | |
| | | AGEEPGYPRFKGRDRFAGFGFKEYG | | | |
| | | NGFKIDGRRLKLSGIDRIAVRWHRA | | | |
| | | LEGTIKTARISCRAGKGFVSFACAVE | | | |
| | | RPEPLPKTGKDIGVDVGLLRLATLSD | | | |
| | | GEPVENPRWYRTLLRELRVLGRKIS | | | |
| | | RAVLGGRNRRKLVRRLQRLLAKVA | | | |
| | | NSRKDFLNKFADTLIKRFDRIVLEDL | | | |
| | | RVAALACGRFALSILDAGRSYLVARL | | | |
| | | AHKAESAGREVVLVDPAYTSKTCSG | | | |
| | | CGTVFEHLSLSDRWISCACGVSLDR | | | |
| | | DHNAAINILRRGRNRPLGAKLLAGG | | | |
| | | VRPEAAPL | | | |
| Y1_Tnp | MBV8078224.1 | MRRVPRLRVSTRPFSSGALVPRVECP | 1535 | Transposase | Planctomycetaceae |
| | | DPLDRESRRDERRIHLVVDHLIGCPT | | IS200 like | bacterium |
| | | RRKPVLVGDGARDCRALSEAGCHD | | | |
| | | HGWTIEDSAIQPDHVHLFIRAWPKD | | | |
| | | SAADVLEAVQGVPAHELRAKSPHLR | | | |
| | | ETPSLWTRPSFASTAGNVSQGTIRRS | | | |
| | | VEAQEGMEMFQAFVSRLDPGASHR | | | |
| | | RRPEAVRETCRRFYNTPLRRRKDAS | | | |
| | | ELRGVSITKTEQPRPVEVEKDTSPYA | | | |
| | | SGIHSHILRAVVADLDDAPRASFRRV | | | |
| | | EAGEEPGYPRFKGRDRFAGFGFKEY | | | |
| | | GDGFTIDGRRPKLSGIGRIAVRWHR | | | |
| | | ALEGTIETARISCRAGKWSVSLACAV | | | |
| | | EGPGPLPETGKEIGVDVGLLRLATLS | | | |
| | | DGAPVEGPRWYRTIPRELRVPRRKIS | | | |
| | | RAVLGGRDRRELVRRLRRSPAEVAN | | | |
| | | ARKDFLNKFADELIKRLDRIALEDLR | | | |
| | | VAAPACGRFALSILDAGRSYLVSRLA | | | |
| | | HKAESAGREVVLVDPASTSKTCSGC | | | |
| | | GRVFEHLSPSDRWISCACGVSLDRD | | | |
| | | HNAAINILQRGRNRPMGAKLSAGRV | | | |
| | | CPEAAPL | | | |
| Y1_Tnp | MBV8077914.1 | MEYRRDEHRLHLVVYHHIWCPKRR | 1536 | Transposase | Planctomycetaceae |
| | | KPVLVGDLARDCRALIEAKCNDLG | | IS200 like | bacterium |
| | | WTIEDLAIQPDHVHLFIRVWPKDSA | | | |
| | | ADVLKAVKGVPAHAPRKRYPHLRK | | | |
| | | TPSLWTRSDFASTAGNVSRETIRRSIE | | | |
| | | AQKGLEMIRTHILPCTIPRARADELN | | | |
| | | RASGAIYTGILVAHWRLVRKKGLWL | | | |
| | | SEASGTRWSDTRTDARMHAHAIDA | | | |
| | | AQQGFYKACETARGLRQAGSAEAK | | | |
| | | FPYHRKKFRTTVWKNSGIKRAGDIL | | | |
| | | RLSGGGRTKEEREKRAVKVAIPPPLR | | | |
| | | DCLRFLEVRLVYDQRARRYTWHVV | | | |
| | | VENGLKPKPAPGRNVVSVDLGEIHP | | | |
| | | AVVGDKEHAIVITCRERRHQSQGHA | | | |
| | | KRLAKIQRAISRKKKDSRRRQRLVR | | | |
| | | AKARMNAKHRQVLRDIEHKVSREI | | | |
| | | VKCAAERQAGTIVIGDIRDIADGIDC | | | |
| | | GAVHNGRMSRWNHGKIRTYVAYKA | | | |
| | | AAEGIKLPPPVDEAYTTQTCPSCGH | | | |
| | | RHKPRGRTFRCPSCGLQAHRDIVGQ | | | |
| | | INILSRFLEGDVGKLPAPAEIKHRIPH | | | |
| | | NLRVMRRCRDTGQGGAL | | | |
| Y1_Tnp | MBV8558358.1 | MEYRRDEHRLHLVVYHHIWCPKRR | 1537 | Transposase | Planctomycetaceae |
| | | KPVLVGDLARDCRALIEAKCNDLG | | IS200 like | bacterium |
| | | WAIENLAIQPDHAHLFIRVWPKDSA | | | |
| | | ADVLKAVKGVPAHAPRKRYPHLRK | | | |
| | | TPSLWTRSDFASTAGNVSRETIRRSIE | | | |
| | | AQKGLEMIRTHILPCTIPRARADELN | | | |
| | | RASGAIYTGILVAHWRLVRKKGLWL | | | |
| | | SEASGTRWSDTRTDARMHAHAIDA | | | |
| | | AQQGFYKACETARGLRQAGIAEAK | | | |
| | | FPYHRKKFRTTVWKNSGIKRAGDIL | | | |
| | | RLSGGGRTKEEREKRAVKVAIPPPLR | | | |
| | | DCLRFLEVRLVYDQRARRYTWHVV | | | |
| | | VENGLKPKPAPGRNVVSVDLGEIHP | | | |
| | | AVVGDKEHAIVITCRERRHQSQGHA | | | |
| | | KRLAKIQRAISRKKKDSRRRQRLVR | | | |
| | | AKARMNAKHRQVLRDIEHKVSREI | | | |
| | | VKCAAERQAGTIVIGDIRDIADGIDC | | | |
| | | GAVHNGRMSRWNHGKIRTYVAYKA | | | |
| | | AAEGIKLPPPVDEAYTTQTCPSCGH | | | |
| | | RHKPRGRTFRCPSCGLQAHRDIVGQ | | | |
| | | INILSRFLEGDVGKLPAPAEIKHRIPH | | | |
| | | NLRVMRRCRDTGQGGAL | | | |
| Y1_Tnp | MBV8610280.1 | MEHRRDEHRIHLVVDHLIGCPTRRK | 1538 | Transposase | Singulisphaera sp. |
| | | PVLVGDVARGCRALSEAKGNELGWI | | IS200 like | |
| | | IENLAIQPDHVHPVVRVWPKDSAAD | | | |
| | | VPKAVKGVTAHELRAKSPHLRKTPS | | | |
| | | LWTRSSFASTAGNVSQETIRRSIEAQ | | | |
| | | KGMEMIRTHILPCTIPRARADELNRA | | | |
| | | SGEIYTGILVAHWRLVRKKRLWLSE | | | |
| | | GAGRRWSDTRTDARMHAHSIDAAQ | | | |
| | | EGFYKACDVTRALRRAGSAEAKFP | | | |
| | | YRRKKFRTTVWKTSGIQREGDILRL | | | |
| | | SGGGRTKKERDERAVVVPIPERLRD | | | |
| | | CLRFLEVRLVYDKYARRYTWHVVV | | | |
| | | ENGLKPKPAPGANVVSVDLGEIHPA | | | |
| | | VVGDTERAIVVTCRERRSRSQGHAK | | | |
| | | RLATISRAIARKAKTSRRRRRLVRAR | | | |
| | | VRMKAKHARILRDIEHKVSREVVAF | | | |
| | | AAERQAGTIVIGDVRDIADGVDCGA | | | |
| | | EHNGRMSRLNHGKIRAYIEYKAAAE | | | |
| | | GIKVELVDEHHTTKTCPGCGQRHKP | | | |
| | | RGRTYRCPSCRFQAHRDVVGQVNIL | | | |
| | | SRFLEGDVGRIPAPADVKYRIPRNVR | | | |
| | | VMRRRCGHQPGSNPRSPGATPWNL | | | |
| | | G | | | |
| Y1_Tnp | MBV8318640.1 | MRRVPRLRVSTRPFSSGALVPRVECP | 1539 | Transposase | Planctomycetaceae |
| | | DPLDRESRRDERRIHLVVDHLIGCPT | | IS200 like | bacterium |
| | | RRKPVLVGDGARDCRALSEAGCHD | | | |
| | | HGWTIEDSAIQPDHVHLFIRAWPKD | | | |
| | | SAADVLKAVQGVPAHELRAKSPHLR | | | |
| | | ETPSLWTRPSFASTAGNVSQGTIRRS | | | |
| | | VEAQEGMEMFQAFVSRLDPGASHR | | | |
| | | RRPEAVRETCRRFYNTPLRRRKDAS | | | |
| | | ELRGVSITKTEQPRPVEVEKDTSPYA | | | |
| | | SGIHSHILRAVVADLDDAPRASFRRV | | | |
| | | EAGEEPGYPRFKGRDRFAGFGFKEY | | | |
| | | GDGFTIDGRRPKLSGIGRIAVRWHR | | | |
| | | ALEGTIETARISCRAGKWSVSLACAV | | | |
| | | EGPGPLPETGKEIGVDVGLLRLATLS | | | |
| | | DGAPVEGPRWYRTIPRELRVPRRKIS | | | |
| | | RAVLGGRDRRELVRRLRRSPAEVAN | | | |
| | | ARKDFLNKFADELIKRLDRIALEDLR | | | |
| | | VAAPACGRFALSILDAGRSYLVSRLA | | | |
| | | HKAESAGREVVLVDPASTSKTCSGC | | | |
| | | GRVFEHLSPSDRWISCACGVSLDRD | | | |
| | | HNAAINILQRGRNRPLGAKPLAGGV | | | |
| | | RPEAAPLEWVRSVTGRLSRRRGSRR | | | |
| | | GCGRRSY | | | |

TABLE 1
Strains

| Strain ID | Description | NCBI accession (and/or description) |
|---|---|---|
| sSL0026 | E. coli BL21(DE3) | CP001509.3 |
| sSL0810 | E. coli MG1655 | U00096.3 |
| sSL0410 | E. coli NEB Turbo | Escherichia coli strain from New England Biolabs (catalogue |
| | | #C2984) |
| sSL3690 | Enterobacter sp. BIDMC93 | KQ089962.1 |
| sSL3710 | Enterobacter cloacae AR_136 | CP021902.1 |
| sSL3711 | Enterobacter cloacae AR_154 | CP029716.1 |
| sSL3712 | Enterobacter cloacae AR_163 | CP021749.1 |
| sSL3860 | Enterobacter sp. BIDMC93 knockout of | KQ089962.1 (replacement of 1,706,193-1,709,444 with cmR |
| | fliC, tldR, and guide | under cat promoter) |
| sSL3864 | Enterobacter sp. BIDMC93 knockout of | KQ089962.1 (replacement of tldR plus its promoter, and guide |
| | tldR plus its promoter, and guide | with cmR under cat promoter) |
| sSL3866 | Enterobacter sp. BIDMC93, non- | KQ089962.1 (replacement of 1,709,375-1,709,394 with |
| | targeting guide mutant (NT-guide) | GstTnpB2 (ISGst3) guide and cmR under cat promoter) |
| sSL3868 | Enterobacter sp. BIDMC93 cmR knock | KQ089962.1 (knock in of cmR downstream of tldR under cat |
| | in downstream of tldR | promoter) |
| sSL3870 | Enterobacter sp. BIDMC93 knockout | KQ089962.1 (replacement of tldR with cmR under cat |
| | tldR | promoter) |
| sSL3872 | Enterobacter sp. BIDMC93 knockout | KQ089962.1 (replacement of prophage encoding tldR with |
| | the whole prophage | cmR under cat promoter) |
| sSL3876 | Enterobacter sp. BIDMC93 knockout of | KQ089962.1 (replacement of FliC with cmR under cat |
| | fliC | promoter) |
| sSL3902 | Enterobacter sp. BIDMC93 knockout of | KQ089962.1 (replacement of tldR plus its promoter, and guide |
| | tldR plus its promoter, and guide, with | with cmR under cat promoter), transformed with pSL6208 |
| | rescue plasmid | |
| sSL3580 | E. coli K-12 MG1655, Stanley Qi strain, | Derivative of strain NC_000913.3 |
| | mRFP-sfGFP integrated into genome + | |
| | fSL0213 (CmR_TldR-targets-F-strand) | |
| | recombineered in between mRFP and | |
| | sfGFP | |
| sSL3761 | E. coli K-12 MG1655, GFP integrated | Derivative of strain NC_000913.3 |
| | into genome, derived from Stanley Qi | |
| | strain sSL0677. Recombineered mRFP | |
| | and the KanR marker out of this strain, | |
| | replacing them with CmR | |

TABLE 2
Description and sequence of plasmids

| | | | Plasmid |
|---|---|---|---|
| | | | Sequence |
| ID | Plasmid Name | Description | SEQ ID NO |
| pSL0007 | pCDFDuet-1 | Empty Vector | 1385 |
| pSL0008 | pCOLADuet-1 | Empty Vector | 1386 |
| pSL0001 | pUC57 | Empty Vector | 1387 |
| pSL5555 | pCDF_Ecl_ωRNA(tSL0620)_FLAG-TldR | 3xFLAG-tag TnpB pEffector plasmid ChIP-seq; TnpB pEffector | 1388 |
| | | plasmid for plasmid cleavage assay, targeting ωRNA | |
| pSL5556 | pCDF_Lec_ωRNA(tSL0620)_FLAG-TldR | 3xFLAG-tag TnpB pEffector plasmid ChIP-seq; TnpB pEffector | 1389 |
| | | plasmid for plasmid cleavage assay, targeting ωRNA | |
| pSL5557 | pCDF_Eko2_ωRNA(tSL0620)_FLAG-TldR | 3xFLAG-tag TnpB pEffector plasmid ChIP-seq; TnpB pEffector | 1390 |
| | | plasmid for plasmid cleavage assay, targeting ωRNA | |
| pSL5558 | pCDF_Eho_ωRNA(tSL0620)_FLAG-TldR | 3xFLAG-tag TnpB pEffector plasmid ChIP-seq and RIP-seq; | 1391 |
| | | TnpB pEffector plasmid for plasmid cleavage assay, targeting | |
| | | ωRNA | |
| pSL4618 | pCDF_Gst3_ωRNA(tSL0530)_FLAG- | 3xFLAG-tag TnpB pEffector plasmid ChIP-seq; TnpB pEffector | 1392 |
| | TnpB2(D196A) | plasmid for plasmid cleavage assay, targeting ωRNA | |
| pSL5552 | pCDF_Kpi_ωRNA(tSL0620)_FLAG-TldR | 3xFLAG-tag TnpB pEffector plasmid ChIP-seq; TnpB pEffector | 1393 |
| | | plasmid for plasmid cleavage assay, targeting ωRNA | |
| pSL5553 | pCDF_Eco_ωRNA(tSL0620)_FLAG-TldR | 3xFLAG-tag TnpB pEffector plasmid ChIP-seq; TnpB pEffector | 1394 |
| | | plasmid for plasmid cleavage assay, targeting ωRNA | |
| pSL5554 | pCDF_Eko1_ωRNA(tSL0620)_FLAG-TldR | 3xFLAG-tag TnpB pEffector plasmid ChIP-seq; TnpB pEffector | 1395 |
| | | plasmid for plasmid cleavage assay, targeting ωRNA | |
| pSL5570 | pCDF_Efal_ωRNA(tSL0634)_FLAG-TldR | 3xFLAG-tag TnpB pEffector plasmid ChIP-seq and RIP-seq; | 1396 |
| | | TnpB pEffector plasmid for plasmid cleavage and RFP repression | |
| | | assay, targeting ωRNA | |
| pSL5571 | pCDF_Ero_ωRNA(tSL0634)_FLAG-TldR | 3xFLAG-tag TnpB pEffector plasmid ChIP-seq; TnpB pEffector | 1397 |
| | | plasmid for plasmid cleavage and RFP repression assay, targeting | |
| | | ωRNA | |
| pSL5572 | pCDF_Eca_ωRNA(tSL0634)_FLAG-TldR | 3xFLAG-tag TnpB pEffector plasmid ChIP-seq and RIP-seq; | 1398 |
| | | TnpB pEffector plasmid for plasmid cleavage and RFP repression | |
| | | assay, targeting ωRNA | |
| pSL5573 | pCDF_Emu_ωRNA(tSL0634)_FLAG-TldR | 3xFLAG-tag TnpB pEffector plasmid ChIP-seq and RIP-seq; | 1399 |
| | | TnpB pEffector plasmid for plasmid cleavage and RFP repression | |
| | | assay, targeting ωRNA | |
| pSL5574 | pCDF_Efa2_ωRNA(tSL0634)_FLAG-TldR | 3xFLAG-tag TnpB pEffector plasmid ChIP-seq and RIP-seq; | 1400 |
| | | TnpB pEffector plasmid for plasmid cleavage and RFP repression | |
| | | assay, targeting ωRNA | |
| pSL5575 | pCDF_Tos_ωRNA(tSL0634)_FLAG-TldR | 3xFLAG-tag TnpB pEffector plasmid ChIP-seq; TnpB pEffector | 1401 |
| | | plasmid for plasmid cleavage and RFP repression assay, targeting | |
| | | ωRNA | |
| pSL5576 | pCDF_Ece_ωRNA(tSL0634)_FLAG-TldR | 3xFLAG-tag TnpB pEffector plasmid ChIP-seq and RIP-seq; | 1402 |
| | | TnpB pEffector plasmid for plasmid cleavage and RFP repression | |
| | | assay, targeting ωRNA | |
| pSL5577 | pCDF_Esa_ωRNA(tSL0634)_FLAG-TldR | 3xFLAG-tag TnpB pEffector plasmid ChIP-seq and RIP-seq; | 1403 |
| | | TnpB pEffector plasmid for plasmid cleavage and RFP repression | |
| | | assay, targeting ωRNA | |
| pSL4369 | pCDF_Gst3_ωRNA(tSL0496)_TnpB2 | TnpB pEffector plasmid for plasmid cleavage assay, targeting | 1404 |
| | | ωRNA | |
| pSL4740 | pCDF_Gst3_ωRNA(tSL0496)_dTnpB2(D196A) | nuclease-dead TnpB mutant pEffector plasmid for plasmid | 1405 |
| | | cleavage assay, targeting ωRNA | |
| pSL5578 | pCDF_Eco2_ωRNA(tSL0496)_FLAG-TnpB | TnpB pEffector plasmid for plasmid cleavage assay, targeting | 1406 |
| | | ωRNA | |
| pSL6087 | pCDF_Eco2_ωRNA(tSL0638)_FLAG-TnpB | TnpB pEffector plasmid for plasmid cleavage assay, non-targeting | 1407 |
| | | ωRNA | |
| pSL5582 | pCDF_Ece_ωRNA(tSL0496)_FLAG-TnpB | TnpB pEffector plasmid for plasmid cleavage assay, targeting | 1408 |
| | | ωRNA | |
| pSL6089 | pCDF_Ece_ωRNA(tSL0638)_FLAG-TnpB | TnpB pEffector plasmid for plasmid cleavage assay, non-targeting | 1409 |
| | | ωRNA | |
| pSL6037 | pCDF_Eho_ωRNA(tSL0496)_FLAG-TldR | TnpB pEffector plasmid for plasmid cleavage assay, targeting | 1410 |
| | | ωRNA | |
| pSL6017 | pCDF_Eho_ωRNA(tSL0638)_FLAG-TldR | TnpB pEffector plasmid for plasmid cleavage and RFP repression | 1411 |
| | | assay, non-targeting ωRNA | |
| pSL6018 | pCDF_Efal_ωRNA(tSL0638)_FLAG-TldR | TnpB pEffector plasmid for plasmid cleavage and RFP repression | 1412 |
| | | assay, non-targeting ωRNA | |
| pSL5579 | pCDF_Pmi_ωRNA(tSL0496)_FLAG-TnpB | TnpB pEffector plasmid for plasmid cleavage assay, targeting | 1413 |
| | | ωRNA | |
| pSL5580 | pCDF_Sen_ωRNA(tSL0496)_FLAG-TnpB | TnpB pEffector plasmid for plasmid cleavage assay, targeting | 1414 |
| | | ωRNA | |
| pSL5581 | pCDF_Bub_ωRNA(tSL0496)_FLAG-TnpB | TnpB pEffector plasmid for plasmid cleavage assay, targeting | 1415 |
| | | ωRNA | |
| pSL5583 | pCDF_Lac_ωRNA(tSL0496)_FLAG-TnpB | TnpB pEffector plasmid for plasmid cleavage assay, targeting | 1416 |
| | | ωRNA | |
| pSL5584 | pCDF_Ste_ωRNA(tSL0496)_FLAG-TnpB | TnpB pEffector plasmid for plasmid cleavage assay, targeting | 1417 |
| | | ωRNA | |
| pSL5585 | pCDF_Shy_ωRNA(tSL0496)_FLAG-TnpB | TnpB pEffector plasmid for plasmid cleavage assay, targeting | 1418 |
| | | ωRNA | |
| pSL4128 | pCOLA_tSL0496- | pTarget plasmid for plasmid cleavage assay | 1419 |
| | target_TTTAT-TAM | | |
| pSL5888 | pCOLA_tSL0496- | pTarget plasmid for plasmid cleavage assay | 1420 |
| | target_CTTAT-TAM | | |
| pSL5891 | pCOLA_tSL0496- | pTarget plasmid for plasmid cleavage assay | 1421 |
| | target_TTTAA-TAM | | |
| pSL6086 | pCOLA_tSL0620- | pTarget plasmid for plasmid cleavage assay | 1422 |
| | target_GTTAT-TAM | | |
| pSL5889 | pCOLA_tSL0620- | pTarget plasmid for plasmid cleavage assay | 1423 |
| | target_TCCAT-TAM | | |
| pSL5890 | pCOLA_tSL0620- | pTarget plasmid for plasmid cleavage assay | 1424 |
| | target TTGAT-TAM | | |
| pSL6052 | pCOLA_tSL0496- | pTarget plasmid for plasmid cleavage assay | 1425 |
| | target_GTTAT-TAM | | |
| pSL6216 | pCDF_Eho_ωRNA(tSL0642)_FLAG-TldR | TnpB pEffector plasmid for RFP repression assay, targeting | 1426 |
| | | ωRNA, promoter-targeting (forward strand) | |
| pSL6218 | pCDF_Eho_ωRNA(tSL0644)_FLAG-TldR | TnpB pEffector plasmid for RFP repression assay, targeting | 1427 |
| | | ωRNA, promoter-targeting (reverse strand) | |
| pLS6217 | pCDF_Efal_ωRNA(tSL0643)_FLAG-TldR | TnpB pEffector plasmid for RFP repression assay, targeting | 1428 |
| | | ωRNA, promoter-targeting (forward strand) | |
| pSL6219 | pCDF_Efal_ωRNA(tSL0645)_FLAG-TldR | TnpB pEffector plasmid for RFP repression assay, targeting | 1429 |
| | | ωRNA, promoter-targeting (reverse strand) | |
| pSL6220 | pSC101_tSL0642_GTTAT- | pTarget plasmid for RFP repression assay, target on promoter | 1430 |
| | TAM_mRFP | (forward strand) | |
| pSL6222 | pSC101_GTTAT- | pTarget plasmid for RFP repression assay, target on promoter | 1431 |
| | TAM_tSL0644_mRFP | (reverse strand) | |
| pSL6221 | pSC101_tSL0643_TTTAA- | pTarget plasmid for RFP repression assay, target on promoter | 1432 |
| | TAM_mRFP | (forward strand) | |
| pSL6223 | pSC101_TTTAA- | pTarget plasmid for RFP repression assay, target on promoter | 1433 |
| | TAM_tSL0644_mRFP | (reverse strand) | |
| pSL6069 | pCDF_Kpi_ωRNA(tSL0496)_FLAG-TldR | TnpB pEffector plasmid for RFP repression assay, targeting | 1434 |
| | | ωRNA, 5′ UTR-targeting (forward and reverse strand) | |
| pSL6070 | pCDF_Eco_ωRNA(tSL0496)_FLAG-TldR | TnpB pEffector plasmid for RFP repression assay, targeting | 1435 |
| | | ωRNA, 5′ UTR-targeting (forward and reverse strand) | |
| pSL6071 | pCDF_Ekol_ωRNA(tSL0496)_FLAG-TldR | TnpB pEffector plasmid for RFP repression assay, targeting | 1436 |
| | | ωRNA, 5′ UTR-targeting (forward and reverse strand) | |
| pSL6072 | pCDF_Ecl_ωRNA(tSL0496)_FLAG-TldR | TnpB pEffector plasmid for RFP repression assay, targeting | 1437 |
| | | ωRNA, 5′ UTR-targeting (forward and reverse strand) | |
| pSL6073 | pCDF_Lec_ωRNA(tSL0496)_FLAG-TldR | TnpB pEffector plasmid for RFP repression assay, targeting | 1438 |
| | | ωRNA, 5′ UTR-targeting (forward and reverse strand) | |
| pSL6074 | pCDF_Eko2_ωRNA(tSL0496)_FLAG-TldR | TnpB pEffector plasmid for RFP repression assay, targeting | 1439 |
| | | ωRNA, 5′ UTR-targeting (forward and reverse strand) | |
| pSL6075 | pCDF_Eho_ωRNA(tSL0496)_FLAG-TldR | TnpB pEffector plasmid for RFP repression assay, targeting | 1440 |
| | | ωRNA, 5′ UTR-targeting (forward and reverse strand) | |
| pSL6093 | pSC101_tSL0496_GTTAT- | pTarget plasmid for RFP repression assay, target in 5′ UTR | 1441 |
| | TAM_mRFP | (forward strand) | |
| pSL6092 | pSC101_GTTAT- | pTarget plasmid for RFP repression assay, target in 5′ UTR | 1442 |
| | TAM_tSL0496_mRFP | (reverse strand) | |
| pSL5908 | pSC101_tSL0496_TTTAA- | pTarget plasmid for RFP repression assay, target in 5′ UTR | 1443 |
| | TAM_mRFP | (forward strand) | |
| pSL5907 | pSC101_TTTAA- | pTarget plasmid for RFP repression assay, target in 5′ UTR | 1444 |
| | TAM_tSL0496_mRFP | (reverse strand) | |
| pSL6207 | pCDF_Spy_sgRNA(tSL0496)_FLAG- | nuclease-dead SpyCas9 mutant pEffector plasmid for RFP | 1445 |
| | dCas9(D10A, H840A) | repression assay, 5′ UTR-targeting sgRNA (forward and reverse | |
| | | strand) | |
| pSL6046 | pSC101_tSL0496_GGG- | pTarget plasmid for RFP repression assay, target in 5′ UTR | 1446 |
| | PAM_mRFP | (forward strand) | |
| pSL5930 | pSC101_GGG- | pTarget plasmid for RFP repression assay, target in 5′ UTR | 1447 |
| | PAM_tSL0496_mRFP | (reverse strand) | |
| pSL6206 | pCDF_As_crRNA(tSL0586)_dCas12a(D908A) | nuclease-dead AsCas12a mutant pEffector plasmid for RFP | 1448 |
| | | repression assay, 5′ UTR-targeting sgRNA (forward and reverse | |
| | | strand) | |
| pSL5928 | pSC101_tSL0496_TTTA- | pTarget plasmid for RFP repression assay, target in 5′ UTR | 1449 |
| | PAM_mRFP | (forward strand) | |
| pSL5927 | pSC101_TTTA- | pTarget plasmid for RFP repression assay, target in 5′ UTR | 1450 |
| | PAM_tSL0496_mRFP | (reverse strand) | |
| pSL2684 | pSIM6_pSC101_GamBetaExo | pSim temp-inducible lambda red, for recombineering in sSL3690 | 1451 |
| pSL6208 | pCDF_BIDMC93_dFliC_TldR + guide | TldR + guide under native promoter for rescue in sSL3864 | 1452 |
| pSL6724 | | pCDF_Ebr_gRNA-region(native)_conserved-region_FLAG- | 5928 |
| | | dCas12f_RpoE_HTH | |
| pSL6725 | | pCDF_Pum_gRNA-region(native)_conserved-region_FLAG- | 5929 |
| | | dCas12f_RpoE_HTH | |
| pSL6726 | | pCDF_Ata_gRNA-region(native)_conserved-region_FLAG- | 5930 |
| | | dCas12f_RpoE_HTH | |
| pSL6727 | | pCDF_Aru_gRNA-region(native)_conserved-region_FLAG- | 5931 |
| | | dCas12f_RpoE_HTH | |
| pSL6728 | | pCDF_Smi_gRNA-region(native)_conserved-region_FLAG- | 5932 |
| | | dCas12f_RpoE_HTH | |
| pSL6729 | | pCDF_Lpa_gRNA-region(native)_conserved-region_FLAG- | 5933 |
| | | dCas12f_RpoE_HTH | |
| pSL6730 | | pCDF_Sda_gRNA-region(native)_conserved-region_FLAG- | 5934 |
| | | dCas12f_RpoE_HTH1_HTH2 | |
| pSL6731 | | pCDF_Lby_gRNA-region(native)_conserved-region_FLAG- | 5935 |
| | | dCas12f_RpoE_HTH1_HTH2 | |
| pSL6732 | | pCDF_Mri_gRNA-region(native)_conserved-region_FLAG- | 5936 |
| | | dCas12f_RpoE_HTH | |
| pSL6733 | | pCDF_Pdi_gRNA-region(native)_conserved-region_FLAG- | 5937 |
| | | dCas12f_RpoE_HTH1_HTH2 | |
| pSL6734 | | pCDF_Psu_gRNA-region(native)_conserved-region_FLAG- | 5938 |
| | | dCas12f_RpoE_HTH | |
| pSL6735 | | pCDF_Cgl_gRNA-region(native)_conserved-region_FLAG- | 5939 |
| | | dCas12f_RpoE_HTH | |
| pSL6736 | | pCDF_Zpr_gRNA-region(native)_conserved-region_FLAG- | 5940 |
| | | dCas12f_RpoE_HTH | |
| pSL6737 | | pCDF_Cba_gRNA-region(native)_conserved-region_FLAG- | 5941 |
| | | dCas12f_RpoE_HTH | |
| pSL6738 | | pCDF_Pba_gRNA-region(native)_conserved-region_FLAG- | 5942 |
| | | dCas12f_RpoE_HTH | |
| pSL7082 | | pCDF_Ata_wRNA-region(native)_conserved- | 5943 |
| | | region_dCas12f_FLAG-RpoE_HTH | |
| pSL7083 | | pCDF_Aru_wRNA-region(native)_conserved- | 5944 |
| | | region_dCas12f_FLAG-RpoE_HTH | |
| pSL7085 | | pCDF_Lpa_wRNA-region(native)_conserved- | 5945 |
| | | region_dCas12f_FLAG-RpoE_HTH | |
| pSL7087 | | pCDF_Lby_wRNA-region(native)_conserved- | 5946 |
| | | region_dCas12f_FLAG-RpoE_HTH1_HTH2 | |
| pSL7088 | | pCDF_Mri_wRNA-region(native)_conserved- | 5947 |
| | | region_dCas12f_FLAG-RpoE_HTH | |
| pSL7089 | | pCDF_Pdi_wRNA-region(native)_conserved- | 5948 |
| | | region_dCas12f_FLAG-RpoE_HTH1_HTH2 | |
| pSL7092 | | pCDF_Zpr_wRNA-region(native)_conserved- | 5949 |
| | | region_dCas12f_FLAG-RpoE_HTH | |
| pSL7093 | | pCDF_Cba_wRNA-region(native)_conserved- | 5950 |
| | | region_dCas12f_FLAG-RpoE_HTH | |
| pSL7182 | | pCDF_Ata_wRNA-region(tSL0675)_conserved-region_FLAG- | 5951 |
| | | dCas12f_RpoE_HTH | |
| pSL7183 | | pCDF_Ata_wRNA-region(tSL0676)_conserved-region_FLAG- | 5952 |
| | | dCas12f_RpoE_HTH | |
| pSL7184 | | pCDF_Ata_wRNA-region(tSL0677)_conserved-region_FLAG- | 5953 |
| | | dCas12f_RpoE_HTH | |
| pSL7185 | | pCDF_Ata_wRNA-region(tSL0678)_conserved-region_FLAG- | 5954 |
| | | dCas12f_RpoE_HTH | |
| pSL7186 | | pCDF_Ata_wRNA-region(tSL0679)_conserved-region_FLAG- | 5955 |
| | | dCas12f_RpoE_HTH | |
| pSL7187 | | pCDF_Ata_wRNA-region(tSL0675)_conserved- | 5956 |
| | | region_dCas12f_FLAG-RpoE_HTH | |
| pSL7188 | | pCDF_Ata_wRNA-region(tSL0676)_conserved- | 5957 |
| | | region_dCas12f_FLAG-RpoE_HTH | |
| pSL7189 | | pCDF_Ata_wRNA-region(tSL0677)_conserved- | 5958 |
| | | region_dCas12f_FLAG-RpoE_HTH | |
| pSL7190 | | pCDF_Ata_wRNA-region(tSL0678)_conserved- | 5959 |
| | | region_dCas12f_FLAG-RpoE_HTH | |
| pSL7191 | | pCDF_Ata_wRNA-region(tSL0679)_conserved- | 5960 |
| | | region_dCas12f_FLAG-RpoE_HTH | |
| pSL7142 | | pCDF_Ata_ΔgRNA-region_conserved-region_FLAG- | 5961 |
| | | dCas12f_RpoE_HTH | |
| pSL7465 | | pCDF_Ata_gRNA-region(tSL0679)_Δconserved-region FLAG- | 5962 |
| | | dCas12f_RpoE_HTH | |
| pSL7466 | | pCDF_Ata_AJ23119_AgRNA-region_Δconserved- | 5963 |
| | | region_FLAG-dCas12f_RpoE_HTH | |
| pSL7467 | | pCDF_Ata_gRNA-region(tSL0679)_conserved- | 5964 |
| | | region_ΔdCas12f_FLAG-RpoE_HTH | |
| pSL7468 | | pCDF_Ata_gRNA-region(tSL0679)_conserved-region_FLAG- | 5965 |
| | | dCas12f_ΔRpoE_HTH | |
| pSL7469 | | pCDF_Ata_gRNA-region(tSL0679)_conserved-region_FLAG- | 5966 |
| | | dCas12f_RpoE_ΔHTH | |
| pSL7475 | | pCDF_Ata_gRNA-region(tSL0679)_conserved- | 5967 |
| | | region_dCas12f_RpoE_HTH-FLAG | |
| pSL7476 | | pCDF_Ata_gRNA-region(tSL0679)_conserved- | 5968 |
| | | region_ΔdCas12f_ΔRpoE_HTH-FLAG | |
| pSL7472 | | pCDF_Ata_20nt-guide(tSL0679)_Δconserved-region_FLAG- | 5969 |
| | | dCas12f_RpoE_HTH | |
| pSL7473 | | pCDF_Ata_14nt-guide(tSL0679)_Δconserved-region_FLAG- | 5970 |
| | | dCas12f_RpoE_HTH | |
| pSL7477 | | pCDF_Smi_gRNA-region(tSL0679)_conserved- | 5971 |
| | | region_dCas12f_RpoE_HTH | |
| pSL7478 | | pCDF_Lby_gRNA-region(tSL0690)_conserved- | 5972 |
| | | region_dCas12f_RpoE_HTH | |
| pSL7479 | | pCDF_Mri_gRNA-region(tSL0690)_conserved- | 5973 |
| | | region_dCas12f_RpoE_HTH | |
| pSL7480 | | pCDF_Zpr_gRNA-region(tSL0679)_conserved- | 5974 |
| | | region_dCas12f_RpoE_HTH | |
| pSL7474 | | pCDF_Ata_gRNA(tSL0689)_conserved-region_FLAG- | 5975 |
| | | dCas12f_RpoE_HTH | |
| pSL7456 | | pCOLADuet_Ata_T7_RpoA_RpoB_T7 | 5976 |
| pSL7457 | | pACYCDuet_Ata_T7_T7_RpoC_RpoZ | 5977 |
| pSL7770 | | pSC101_Ata-dCas12f_native_target_strong-RBS_mRFP | 5978 |
| pSL7771 | | pUC19_Ata-dCas12f_native_target_strong-RBS_mRFP | 5979 |
| pSL6740 | | pCDF_Fpl_wRNA-region_FLAG-dTnpB_CsrA | 5980 |
| pSL6741 | | pCDF_Osp_wRNA-region_FLAG-dTnpB_CsrA | 5981 |
| pSL6742 | | pCDF_Fba_wRNA-region_FLAG-dTnpB_CsrA | 5982 |
| pSL6743 | | pCDF_Fpl2_wRNA-region_FLAG-dTnpB_CsrA | 5983 |
| pSL6746 | | pCDF_Psp_wRNA-region_FLAG-dTnpB_CsrA | 5984 |
| pSL6747 | | pCDF_Fpl3_wRNA-region_FLAG-dTnpB_CsrA | 5985 |
| pSL6748 | | pCDF_Isp_wRNA-region_FLAG-dTnpB_CsrA | 5986 |
| pSL6749 | | pCDF_Las_wRNA-region_FLAG-dTnpB | 5987 |
| pSL7312 | | pCDF_Osp_AgRNA_FLAG-TldR_CsrA | 5988 |
| pSL7313 | | pCDF_Osp_AgRNA-downstream_FLAG-TldR_CsrA | 5989 |
| pSL7314 | | pCDF_Osp_AgRNA-upstream_FLAG-TldR_CsrA | 5990 |
| pSL7315 | | pCDF_Osp_gRNA-HDV-downstream_FLAG-TldR_CsrA | 5991 |
| pSL7316 | | pCDF_Osp_wRNA-upstream-and-HDV-downstream_FLAG- | 5992 |
| | | TldR_CsrA | |
| pSL7317 | | pCDF_Osp_wRNA-region_FLAG-TldR_ACsrA | 5993 |
| pSL7318 | | pCDF_Osp_wRNA-region_TldR_FLAG-CsrA | 5994 |
| pSL7319 | | pCDF_Osp_wRNA-region_ΔTIdR_FLAG-CsrA | 5995 |
| pSL7320 | | pCDF_Osp_wRNA-region_TldR_V5-CsrA | 5996 |
| pSL7321 | | pCDF_Osp_wRNA-region_TldR_CsrA-HA | 5997 |

TABLE 3
Genes

| Gene (IS Element) | Protein | NCBI Accession |
|---|---|---|
| tnpB (ISGst3) | GstTnpB2 | WP_047817673.1 |
| cas9 (Spy) | Cas9 | WP_136301537.1 |
| cas12 (As) | Cas12 | WP_021736722.1 |
| tldR (Kpi) | Kpi-TldR | WBG92703.1 |
| tldR (Eco) | Eco-TldR | WP_064735610.1 |
| tldR (Eko1) | Eko1-TldR | WP_193971683.1 |
| tldR (Ecl) | Ecl-TldR | WP_110870855.1 |
| tldR (Lec) | Lec-TldR | AXF62639.1 |
| tldR (Eko2) | Eko2-TldR | WP_023337454.1 |
| tldR (Eho) | Eho-TldR | WP_017692904.1 |
| tldR (Efa1) | Efa1-TldR | WP_002406890.1 |
| tldR (Ero) | Ero-TldR | WP_208930379.1 |
| tldR (Eca) | Eca-TldR | WP_121260685.1 |
| tldR (Emu) | Emu-TldR | WP_034688898.1 |
| tldR (Efa2) | Efa2-TldR | WP_002289328.1 |
| tldR (Tos) | Tos-TldR | WP_123935583.1 |
| tldR (Ece) | Ece-TldR | WP_016251060.1 |
| tldR (Esa) | Esa-TldR | WP_232061298.1 |
| tnpB (Eco2) | Eco2-TnpB | WP_098717298.1 |
| tnpB (Pmi) | Pmi-TnpB | WP_269608765.1 |
| tnpB (Sen) | Sen-TnpB | WP_024186316.1 |
| tnpB (Bub) | Bub-TnpB | WP_059759460.1 |
| tnpB (Ece) | Ece-TnpB | WP_113843517.1 |
| tnpB (Lac) | Lac-TnpB | WP_242450195.1 |
| tnpB (Ste) | Ste-TnpB | WP_028983493.1 |
| tnpB (Shy) | Shy-TnpB | WP_277281207.1 |

TABLE 4
TldRs Referred to herein

| Alias | SEQ ID NO | Organism |
|---|---|---|
| Kpi | 372 | Kalamiella piersonii |
| Eco | 285 | Escherichia coli |
| Eko1 | 62 | Enterobacter kobei |
| Ecl | 86 | Enterobacter cloacae complex sp. |
| Lec | 87 | Leclercia sp. W6 |
| Eko2 | 20 | Enterobacter kobei |
| Eho | 8 | Enterobacter hormaechei |
| Efa1 | 399 | Enterococcus faecalis |
| Ero | 457 | Enterococcus rotai |
| Eca | 442 | Enterococcus casseliflavus |
| Emu | 177 | Enterococcus mundtii |
| Efa2 | 392 | Enterococcus faecium |
| Tos | 443 | Tetragenococcus osmophilus |
| Ece | 121 | Enterococcus cecorum DSM 20682 = ATCC 43198 |
| Esa | 468 | Enterococcus saigonensis |

TABLE 5

| | Species 3- | Protein | | SEQ ID |
|---|---|---|---|---|
| Species name | letter code | name | Protein amino acid sequence | NO |
| Flavonifractor plautii | Fpl | TldR | MASREKQYNVLKLRLYPTSEQAELFEKTFGCCRYL | 497 |
| | | | WNQMLADQQRFYLETGVHFIPTPAKYKKGAPFLKE | |
| | | | VDNQALIQEHNQLSRAFRLFFQNPEAFGHPNFKRK | |
| | | | KDDRDSFTACNHVFTSGPTIYTTRDGIRMTKAGMIR | |
| | | | AVFPRRPQNGWKLKRVTVEKARTGRYYAYVLYESL | |
| | | | VQPPEPVLPVPERTLGLKYSLRHFYVDDQGNRADP | |
| | | | PRWLKQSQEKLVHLQRRLNRMQPGSKNYEEAVLK | |
| | | | YRLLHEHIANQRRDFLHKESRRIANAWDAVCVRGD | |
| | | | DLGAMTDTLIQAGSTVKEAGFGMFREMLCYKLAR | |
| | | | QGKAFIQVDRYLPTTRSCSACGLTRDALHARDYRR | |
| | | | SGWVCPECGAVHDREVNAAKNIKARGLEQFFDLQ | |
| | | | GQDRSA | |
| | | CsrA | MLQLSLRPGEYLTIHGDIVVQLAQLSGSRAFLRVEA | 5998 |
| | | | DRSIPIVRGKVLERSGAPRPECLASLPRSRARKGRD | |
| | | | AVYHWSAERERAVRTMEQLLERMEAGDSREEAQA | |
| | | | LRAQLEHLLPTVWEEELSGQIQALFRSKTAQDV | |
| Oscillibacter sp. 1-3 | Osp | TldR | MAAKRSKSETLRYTTLKVRLYPSAEQAALFEKTFG | 500 |
| | | | CCRYIWNQMLADQQRFYIETDKFFIPTPAKYKAGAP | |
| | | | FLKEVDNQALIQEHNKLGQAFRVFFKSPENFGYPKF | |
| | | | KRKKDDRDSFTVCNHVMGNSETVYTTRDGLRMTK | |
| | | | AGIVRAKFPRRPQGWWKLKRVTVDRTRSGKYYGY | |
| | | | ILYECPEKKPEVVVPTPETTVGLKYSMARFYVADTG | |
| | | | ETADPPHWLKQSQEKLARIQQRLNRMRPGSKNYQE | |
| | | | TVQKYRLLHEHIANQRRDFIHKESRRIANAWDAVC | |
| | | | VRGDDMEQISRITNRGNALEAGFGMFRECLRYKLA | |
| | | | RQGKELLVVDRYFPSTRTCSACGRVMPEEISMKRRT | |
| | | | WTCPQCGAVLKREANAARNIKDQGLAQYFSTRERR | |
| | | | ESA | |
| | | CsrA | MLCLNLTPGEYMTIGDSVVVQLDRISGDRCKLMID | 5999 |
| | | | APREIPVLRGEVLERTGGERPSCVVEGPRWHRREIP | |
| | | | WNRSKAQALAAMRMLLSEMDGRDSNVQALRRQL | |
| | | | DHMFPPEPGREKTELPARASNN | |
| Firmicutes bacterium | Fba | TldR | MARKSRAAEGQVIQYTTLKVRLYPTPAQAELFEKT | 473 |
| | | | FGCCRYIWNQMLSDQQMFYAETGAHFIPTPAKYKK | |
| | | | GAPFLTEVDNQALIQEHNKLSQAFRVFFKRPEAFGH | |
| | | | PNFKKKKTDRDSFTACNHVFESGPTIYTTRDGIRMT | |
| | | | KAGVVKARFSRRAQAWWRLKRITVEKTKTQKYYC | |
| | | | YILYEHSGKQPEPVIPTPETTVGLKYSMRHFYVADD | |
| | | | GTTADPPRWLKQSQEKLVRVQQKLARMEPGSRNYE | |
| | | | EAVQKYRLLHERIANQRRDFLHKESSRIANGWDAV | |
| | | | CMRDDALAEMSKGPLRKDAASSGFRMLRELLQYK | |
| | | | LERQGKRLILLDRYAPTTRVCSVCGQLQDSVDYGA | |
| | | | RTWTCPKCGTVHDREVNAAKNIKLEGLAQFLPTAS | |
| | | | PA | |
| | | CsrA | MLCLSLNQGEYMTIGENVVVQLDHVTGDRCRLVIH | 6000 |
| | | | APKEVPILRGEVLERNGGQRPECVYDGHRYHKKEL | |
| | | | IWNRSKAQALAAMRRLLEEMDGANSDVQALRRQL | |
| | | | NHMFPPADGGAGDSPQTTQFSNG | |
| Flavonifractor plautii | Fp12 | TldR | MKQEKQDGHAEGNRVIQYNTIKVRLCPTPEQEELF | 55 |
| | | | QKTFGCCRYIWNQMLSDHERFYEETDAHFIPTPAK | |
| | | | YKKGAPFLKEVDNQALTQEYNRLSQAFRNFFRDPK | |
| | | | TFGYPKFKRKKDDRDSFTACNQFFGSSATIYATRDA | |
| | | | VRMTKAGLVKAKFSRRPRSGWKLTRLTVERTKTGK | |
| | | | YYGYLLYTCPTYQPEPVEATAERTIGLKYSVSHFYV | |
| | | | ADNGNSADPPRWLRQSQEKLAVVQRKLSRSQPGSQ | |
| | | | NYQELVQKYRLLHEHIANQRRDFLHKESRRIANAW | |
| | | | DAVCIREDSLRAISGKLGGSAVHDTGFGMFRELLRY | |
| | | | KLERQGKQLLEVDRLVPTTKVCSACGAVNETLSIRA | |
| | | | RRWVCPVCGAEHRRGMNAAINIKASGLVKGQSQQ | |
| | | | AAAALPLL | |
| | | CsrA | MLQLSLRPGEYLTIHGDIVVQLAQLSGSRAFLRVEA | 6001 |
| | | | DRSIPIVRGKVLERSGAPRPECLASLPRSRARKGRD | |
| | | | AVYHWNGARKRAIRAMEQILDQMESDGPREEVQA | |
| | | | LRVQLEQLLPTQQEEELSGQIQALFRDQAARNT | |
| Pseudoflavonifractor | Psp | TldR | MKMNDNRRPSAPKRTTQYNTIKIRLYPNQEQEELFQ | 487 |
| sp. | | | RTFGCCRYIWNRMLADHERFYYETDAHFIPTPAKY | |
| | | | KTEAPFLKEVDHQALTQEYNKLSQAFRNFFRNPASF | |
| | | | GYPKFKRKKDDRDSFSACNQVMGNSATIYITQDAV | |
| | | | RMTKAGLVRAKFPRRPRSGWKLTRITVERTKTGKY | |
| | | | YGYLLFACPVHAPEPVKPTADTTIGLKYSLTHFYVR | |
| | | | DDGITADPPRWLRQSQDKVSSIQEKLNRMQPGSRN | |
| | | | YREMVQKYRLLHEHIANQRRDFLHKESRRIANDW | |
| | | | DAVCIRDDSLKAISEELGGSDIHDTGFGMFREMLRY | |
| | | | KLDRQGKQLLEVGRFDPTTKVCSVCGAINETLSPK | |
| | | | ARHWVCPVCGAEHKRGKNAAVNIKAHGLACYQN | |
| | | | KQVAEAVS | |
| | | CsrA | MLSLSLLPDEYLSINNGQIIVHLIRVAGGRAHLRIEA | 6002 |
| | | | DRSVPIVRGALLEREGAARPECLTPPPRRNPGHRRD | |
| | | | HLYLWNDDRERAVRAIQQSIDRLEQTGETAEADILR | |
| | | | TQLNRLIPTFWEEEKLPRRLREQTADSV | |
| Flavonifractor plautii | Fp13 | TldR | MGHRETVGQAIQYNTIKVRLYPSVNQKELFQKTFG | 496 |
| | | | CCRYIWNQMLSDHERFYLETDVHFIPTPAKYKKSAP | |
| | | | FLSKVDNQALIQEHNKLSQAFRNFFRNPGAFGYPRF | |
| | | | KRKKDDRDTFTACNQFFGRSATIYITQNAVRMTKV | |
| | | | GLVRAVFPRRPRSGWRLTRITVERTRTDKYYGYLLY | |
| | | | ACPVRPPQPVTPTEETTVGLNYSVSRFYVADDGTAA | |
| | | | DPPRWLRQSQDQLCQIQRQLCRMQKGSKNYQEMV | |
| | | | QKYRLLHEHIANQRRDFLHKESRRIANEWDAVCVR | |
| | | | SDSLTALAAKTGGGCILDTGFGMFREMLRYKLERQ | |
| | | | GKSLLLVDRFRPTTKVCSVCGYVNEDLPAEALRWR | |
| | | | CPVCGTEHRRERNAAANVKAIGLGRYRTETAAGGI | |
| | | | G | |
| | | CsrA | MLSLQLKSGEYVTIGEEIAVQVFKQSGDSFHVAVKA | 6003 |
| | | | PREVPILRGKVLERTERRPDGLYRRPPQSPSEQRHN | |
| | | | AKRLEAWTLKKAMREQIRAAAMEDLLEVAQYIEDL | |
| | | | AVDRSCCVERQRLSVLGVRITKAVSVLNSTGGGM | |
| Intestinibacillus sp. | Isp | TldR | MAQTKTWNTTIKVRLDPTPAQAAFFDENFNCCRYL | 39 |
| | | | WNQMLSDQIRFYTETDAHFIPTPAKYKKDAPFLKE | |
| | | | ADSNALVSVHQNLHKAFQRFFSNPSRYRHPTFKSK | |
| | | | KRCKNSYTTYCQYYRSGKGTSIYLTKDGIRLPKAGL | |
| | | | VKARLHRRPLHWWTLKTATISKTSSGKYYCSLVFA | |
| | | | YTTKPSRQIPPTPETTLGLNYSLSHFYIDSNGHAADP | |
| | | | PHWLARSQDKLRYMQQQLARMQPGSRNYEQQLY | |
| | | | KIQRLHEHISNQRKDFLHKESRRIANAWDAVCVKD | |
| | | | TNLVKMSQAIKLGHVMDAGYGRFRSYLQYKLERL | |
| | | | GKPYIVVEKYFPSTKTCHHCGSVNEALPAGAKRWT | |
| | | | CPICGTTLDRAKNAAQNLRDQGLVQYSASQRQRAS | |
| | | | A | |
| | | CsrA | MLSLQIKSGEYITIGENVVIQVFQRSGSQFRLAIQAP | 6004 |
| | | | RELSIVRGEVRERQGNARPESVCDPSNGPMVRQQA | |
| | | | RRMQRLEARQKAAAIRADAVQQLRTLLHQSDAGA | |
| | | | AIELAVALQLERLEQSEILATEGGATRDGTNKNLEH | |
| | | | DHQSAP | |

TABLE 6

| | | | | TAM |
|---|---|---|---|---|
| | gRNA sequence: | | | flanking |
| | scaffold + native | | | putative |
| | guide (RIP-seq footprint | 20 nt native | | native |
| gRNA | or inferred from MSA) | guide sequence | Original gRNA region | target |
| Fpl_ | ATATGCCGGCCGGGACACCGGTG | AATATTATGA | GTGCGGCGCGGTTCATGACCG | AGCAC |
| TldR | AACGCCCATGATTCCGGCTGGAG | CGATGTTTTT | TGAGGTCAACGCGGCAAAAAA | |
| native | AGCGTGGATGGAACGCCGGTGGG | (SEQ ID NO: | CATCAAAGCCCGTGGGCTGGA | |
| | GAGCCTCCGCCCTTCCATCCACAT | 6012) | ACAGTTTTTTGATTTACAGGGG | |
| | TCTCCGGACCGCCGAGTGGGAAA | | CAGGACAGGAGCGCCTGACCC | |
| | CGAGAGGCCGCATCCGTGCGGCT | | TTTCCAGGCGTCCTGTGCTCCG | |
| | GAAGCCTGTAAGCAGGCGTCCAC | | CCTTGACAATATGCCGGCCGGG | |
| | AATATTATGACGATGTTTTT (SEQ | | ACACCGGTGAACGCCCATGAT | |
| | ID NO: 6005) | | TCCGGCTGGAGAGCGTGGATG | |
| | | | GAACGCCGGTGGGGAGCCTCC | |
| | | | GCCCTTCCATCCACATTCTCCG | |
| | | | GACCGCCGAGTGGGAAACGAG | |
| | | | AGGCCGCATCCGTGCGGCTGA | |
| | | | AGCCTGTAAGCAGGCGTCCAC | |
| | | | AATATTATGACGATGTTTTTGCA | |
| | | | CGCGGCGCATGGATGCGCGCC | |
| | | | GCTGGCCACAGGAAGGAGATC | |
| | | | CCCC (SEQ ID NO: 6019) | |
| Osp_ | CTATACCGGTCGGAACGCCGGAG | AACATTATG | ATGCGGCGCGGTCCTGAAACG | AACAC |
| TldR | AAGGCCCATGCTTGCGGGAAACG | ACGATGAAA | GGAGGCAAATGCCGCCAGGAA | |
| native | TTGAGCGAAGCACAATTGAAAAA | GA (SEQ ID | CATCAAGGACCAGGGGCTGGC | |
| | GTTGCGTTTCGTTCAGCGTTTCCA | NO: 6013) | CCAATATTTCAGTACGCGGGAG | |
| | TAACCAGGATGTTGGGAAACGAG | | CGGCGCGAAAGCGCGTGAAGC | |
| | AAGATGTGTTCGCACATCCAAAG | | TCTTTGTATTGTCTCAGCTATAC | |
| | CCCTGCGGGGCGTTCCAAACATTA | | CGGTCGGAACGCCGGAGAAGG | |
| | TGACGATGAAAGA (SEQ ID NO: | | CCCATGCTTGCGGGAAACGTT | |
| | 6006) | | GAGCGAAGCACAATTGAAAAA | |
| | | | GTTGCGTTTCGTTCAGCGTTTC | |
| | | | CATAACCAGGATGTTGGGAAA | |
| | | | CGAGAAGATGTGTTCGCACAT | |
| | | | CCAAAGCCCTGCGGGGCGTTC | |
| | | | CAAACATTATGACGATGAAAG | |
| | | | ATCGTCGATGCCGTGAAGCGC | |
| | | | GGCTTGAAGGGCGGACGGAAG | |
| | | | GATGATCGTGCCGCCAGACAA | |
| | | | AAAAGGAGGAATCCTC (SEQ | |
| | | | ID NO: 6020) | |
| Fba | CGATGCCGGTCGGGACGCCGGGG | AATATTATGA | CTGCCCAAAGTGCGGGACGGT | AGCAC |
| TldR | AATGCCCGCGATTCGGCTGGAGG | CGATGAAAA | CCACGACCGGGAGGTCAACGC | |
| native | GCGTTGAGCGAAGCTCAAAAGAA | A (SEQ ID NO: | AGCAAAAAACATCAAGCTGGA | |
| | TTTGTGTTTCGCTCAGCGTTTTCC | 6014) | AGGTTTGGCGCAGTTTTTACCA | |
| | AGGCCGGAGGGTGGGAAACGAG | | ACCGCGAGCCCAGCGTAAGCG | |
| | AGGGCGCGTGCGCGCGCCTGAAA | | GGTCGCGGTTGGAGAGGAGGA | |
| | GCCTGTGACAGGGCTGTCCAAAA | | GCAAGGGAGTGAAGTGAGGA | |
| | TATTATGACGATGAAAAA (SEQ ID | | ATGCCCGGCAGGGTGTTCCGA | |
| | NO: 6007) | | ACGAAATGGACTTTGCTCCGA | |
| | | | CGATGCCGGTCGGGACGCCGG | |
| | | | GGAATGCCCGCGATTCGGCTG | |
| | | | GAGGGCGTTGAGCGAAGCTCA | |
| | | | AAAGAATTTGTGTTTCGCTCAG | |
| | | | CGTTTTCCAGGCCGGAGGGTG | |
| | | | GGAAACGAGAGGGCGCGTGC | |
| | | | GCGCGCCTGAAAGCCTGTGAC | |
| | | | AGGGCTGTCCAAAATATTATGA | |
| | | | CGATGAAAAATGGCGGAAGGA | |
| | | | TGGAGTGCCGGACCGCCTTGT | |
| | | | GAAAAAGGAGTTGAAAAA | |
| | | | (SEQ ID NO: 6021) | |
| Fp12 | ATCTGCCGGCTGGAACACCGGTG | AATATTATGA | AGCGGAGCACCGGCGGGGGAT | AGCAC |
| TldR | AAGGCCCATGCTCCCGGTCTGGG | CGATGTTTAC | GAACGCTGCCATCAACATCAA | |
| native | AGCGTTGAGCGAAGCGGCATACA | (SEQ ID NO: | GGCCAGCGGACTGGTAAAAGG | |
| | GGAAGATGCCGTTTCCCTCAGCGT | 6015) | CCAGAGCCAGCAGGCTGCGGC | |
| | TCTCCGGGCGGGGGACGTTGGGA | | GGCCCTTCCACTCCTCTGATAC | |
| | AACGAGAGGGCGCCTCTCCCAAC | | AACAACATCTGCCGGCTGGAA | |
| | CGGGAGCCGCGCCGGAAGCCCGG | | CACCGGTGAAGGCCCATGCTC | |
| | TCAGACGGGCGTTCCCAATATTAT | | CCGGTCTGGGAGCGTTGAGCG | |
| | GACGATGTTTAC (SEQ ID NO: | | AAGCGGCATACAGGAAGATGC | |
| | 6008) | | CGTTTCCCTCAGCGTTCTCCGG | |
| | | | GCGGGGGACGTTGGGAAACGA | |
| | | | GAGGGCGCCTCTCCCAACCGG | |
| | | | GAGCCGCGCCGGAAGCCCGGT | |
| | | | CAGACGGGCGTTCCCAATATTA | |
| | | | TGACGATGTTTACACACAAGG | |
| | | | AGATCCCC (SEQ ID NO: 6022) | |
| Psp | TGTTGCCGGCCGGAACACCGGAG | AACATTATG | GTGCGGCGCGGAGCACAAACG | Unknown |
| TldR | AAGGCCCATGCTTCCCGTTGGGG | ACGATGATC | AGGGAAAAACGCCGCCGTCAA | |
| native | AGCGTTGAGCGAAGCGGCGGACA | AT (SEQ ID | TATCAAAGCCCATGGGCTGGC | |
| | GTACGCTGCCGTTTCCCTCAGCGT | NO: 6016) | GTGTTACCAGAATAAACAGGTT | |
| | TCTCCAATCCGCGGGAGATCGTTG | | GCGGAGGCAGTTTCATAACCTT | |
| | GGAAACGAGAAGATGCCTCCCCT | | CCTGCAAAAAGTGATGTTGCC | |
| | CTGGGGCCGCATCCGAAGCCCGG | | GGCCGGAACACCGGAGAAGG | |
| | CAGGGCGTTCCAAACATTATGACG | | CCCATGCTTCCCGTTGGGGAGC | |
| | ATGATCAT (SEQ ID NO: 6009) | | GTTGAGCGAAGCGGCGGACAG | |
| | | | TACGCTGCCGTTTCCCTCAGCG | |
| | | | TTCTCCAATCCGCGGGAGATCG | |
| | | | TTGGGAAACGAGAAGATGCCT | |
| | | | CCCCTCTGGGGCCGCATCCGA | |
| | | | AGCCCGGCAGGGCGTTCCAAA | |
| | | | CATTATGACGATGATCATCCAA | |
| | | | GATGATCGTACCAGCGCAAAG | |
| | | | GAGTGCGCTGTCTCATCAGAC | |
| | | | ACAGGAGGTCTTTTTGT (SEQ | |
| | | | ID NO: 6023) | |
| Fp13 | TATGGCCGGTCGGGACGCCGGTG | AATATTATGA | CTGCGGGACGGAGCACAGGCG | AGCAC |
| TldR | AACGCCCGCATACCCTCCAATAGG | CGATGATAA | CGAACGCAACGCAGCGGCGAA | |
| native | CGGGGCCGCGGGAAACGAGAGG | C (SEQ ID NO: | TGTCAAAGCCATCGGCCTGGG | |
| | CCGCCTCCCCCTTACCGGGGCCGC | 6017) | CCGGTACCGCACGGAGACGGC | |
| | GGCGGAAGCCCGGACAGCCGGGC | | AGCCGGCGGAATTGGGTAGCA | |
| | GTCCAAAATATTATGACGATGATA | | CGAAGTGCCCCGCAGGTAGCG | |
| | AC (SEQ ID NO: 6010) | | GGGGTACTCCATATGGCCGGTC | |
| | | | GGGACGCCGGTGAACGCCCGC | |
| | | | ATACCCTCCAATAGGCGGGGCC | |
| | | | GCGGGAAACGAGAGGCCGCCT | |
| | | | CCCCCTTACCGGGGCCGCGGC | |
| | | | GGAAGCCCGGACAGCCGGGCG | |
| | | | TCCAAAATATTATGACGATGAT | |
| | | | AACCCTTAATCTGGTTTCATCC | |
| | | | GGCCTAGCGCAGCCGGTTGAA | |
| | | | ATATAGGAAGCGTGCGGAGAG | |
| | | | AGCAGGTGCCAAAGGCCACGG | |
| | | | AGCCTGCCGAACCGCACTAAA | |
| | | | CAACCAACCGCACAAGAAGGA | |
| | | | TACTGTGCGGCGACCAGCAAA | |
| | | | GTCCCTTATCATTTCATATTGAA | |
| | | | AGGAAGCAATTTGA (SEQ ID | |
| | | | NO: 6024) | |
| Isp | TAGGACCGGTTGGGACACCGGTG | GAGAATCCA | CTGTGGTACAACGCTTGACCG | ATCAT |
| TldR | TCTGCCCATGACACAAGGATGCG | ACATTATATT | TGCCAAAAATGCGGCGCAAAA | |
| native | GAAGCGTTGATTGGGTCGAAAGA | T (SEQ ID NO: | CCTGCGCGATCAGGGCCTTGTA | |
| | ACGGGTTTCTTTCGACGCGATCAG | 6018) | CAATATTCCGCTTCCCAGAGGC | |
| | CGTTTCCGTTCGGATGTGAAGTGG | | AGCGCGCTTCGGCCTGACTCT | |
| | GAACAAAAGACGGCAACCTCCGC | | GTCATAGGACCGGTTGGGACA | |
| | CATCCCCAGGAGCGCGCCCGAAG | | CCGGTGTCTGCCCATGACACA | |
| | CCCGGCATGCCGGGCAGTCCCCG | | AGGATGCGGAAGCGTTGATTG | |
| | AGAATCCAACATTATATTT (SEQ ID | | GGTCGAAAGAACGGGTTTCTT | |
| | NO: 6011) | | TCGACGCGATCAGCGTTTCCGT | |
| | | | TCGGATGTGAAGTGGGAACAA | |
| | | | AAGACGGCAACCTCCGCCATC | |
| | | | CCCAGGAGCGCGCCCGAAGCC | |
| | | | CGGCATGCCGGGCAGTCCCCG | |
| | | | AGAATCCAACATTATATTTGTTT | |
| | | | TCATCCGGCCGACGGGTCGGT | |
| | | | TGAAATAGAGTTCATGCGTCCC | |
| | | | ATGATTTTGGGCAGTTCGGCCG | |
| | | | AATGTCCGAAGTTCCATGACGC | |
| | | | TTCCACGACCTGTGCTGGGGT | |
| | | | AAATTGGATGGCCCCGTCACA | |
| | | | CTAATCAGATTTAGGAGGAACC | |
| | | | TAATT (SEQ ID NO: 6025) | |

TABLE 7

| | Species | | | |
|---|---|---|---|---|
| | 3-letter | Protein | | SEQ ID |
| Species name | code | name | Protein amino acid sequence | NO |
| Empedobacter | Ebr | dCas12f | MMEKSTLILTRKIQLIVDLPTQEERQEVLEILYKWRNRCYR | 6026 |
| brevis | | | AANLIVSHLYIQAMVKEFLYLSEGVIHKLVDEKKDEAGILQ | |
| | | | RSRINTTYRVISDRFKGEIPMNILSCLNSRLQSTFNKDYQEY | |
| | | | WRGEASLKNFKRDMAFPFGLEGISKLSYHPEKKSFCFRLF | |
| | | | QLPFKTYLGRDFTGNKKLLEQVINDEVKLCTSQIKIEKGKI | |
| | | | FWLAVVEIEKENHQLQPEKIAEASLSLEYPLVVKVGKSRLS | |
| | | | IGTKEEFLYRRLAIQASRKRMQAGVSYAKSGKGRTRKLKA | |
| | | | LEKMSELERNYVHNRLHVYSRRLIDFCINNKAGTLILLDQ | |
| | | | EEKMELAKEEEFVLRNWSYYELMTKIKYKADKAGIELIIA | |
| | | RpoE | MLEKEFELLKKGNSTALERIYVRYNKRIFWFGKQLIKDEF | 6043 |
| | | | VVECLLQDVFLKLWEYREKIESPDHIFFFLRFVMKYSCYS | |
| | | | HYAKPKNKFFRNVNSIENYENYQSYLAGYDPADVIENLNE | |
| | | | QERQQHYFNEIIKVLPLLSTERKHLIELCLKYGFQYKAIAH | |
| | | | VMGRGITETSNEIKSAIEDLKKILSHQDKLIIKTKTISTEEKQ | |
| | | | EKMTKTQSLILKLRCEHKQSFANIAEELQLSQKEVHNEFIA | |
| | | | AYKFTQQNKVQSLNY | |
| | | HTH | MEKNNYKSNLNKKLCEYITIRFLSNFDNISNNNSQNKYAK | 6060 |
| | | | AVGVTSSTISKISKGDGYNIPLSTIALILKYEKISLEDFFKDF | |
| | | | NKYVNE | |
| Paenimyroides | Pum | dCas12f | MSKTTIKLTRKIELNIDLPTKEQRKEVWEKLYRWQNIYCR | 6027 |
| ummariense | | | AANLTMSHLYVQAMIKDFLYLTEGIKYKLADEKKDPNGM | |
| | | | LQCSHSSSIYRMLSQRFKGEVPTKILNHANYELMNKFKKN | |
| | | | YMDYVNGKRSLDNFKSNTVFPFGIEGFKRFKYNEEIKAFS | |
| | | | FRLYSVPFKTFLGRGFTEKYKLLQQLLSGEVKLCRSRIKLE | |
| | | | KGKIYWLAVFEIPIEVHCLKPDVVAEASLSLEYPISVKVGR | |
| | | | KRLDIGNKEEFLHRRLAIQAAYTRTRESVKYCRGGHGKKR | |
| | | | KLKALDRFKNLEANYVSNRLHEYSRRLIDFCIKHQAGTLV | |
| | | | LLDMQENTDIAKEEQFVLRNWSYYELINKIKYKAEKAGIE | |
| | | | LITT | |
| | | RpoE | MVQDTFLKLWDCREKIQDPMHILFFLQYVIKKSCLSHYNK | 6044 |
| | | | PRNKFFRKVGSLESYENFQNYLAGYDPADVVENLKDQES | |
| | | | QQKMFDLVTSVLPLIKPERKHLINLCLKYGFRYKHIAQVM | |
| | | | GKSTKQTVDEVNRAIEDIKKIVAVRNRNEKKFKPELEQKA | |
| | | | VSERQSQVLKLRCEKKFSFAAIAEQLNLSQKQVHEEFMAA | |
| | | | YKFAQQHKLQSL | |
| | | HTH | MDITKDLQILIGKQLHDIRIKNKQTQNDIAFLTGIDTADVSK | 6061 |
| | | | HEKGKKNLTLKTLMKFATALNIHPKELFNFDFDINRYKTE | |
| | | | Y | |
| Allomuricauda | Ata | dCas12f | MGKSTLKHTRKIQILIDLPTKDEKKEVMDMMYQWRDRCF | 6028 |
| taeanensis* | | | RAANIIVTHLYVQEMIKDFFYLSEGIKYKLADEKKDEKGIL | |
| strain JCM 17757 | | | QRSRMNTTYRVVSDRFKGEMPTNILSTLNHGLISSFNKNR | |
| | | | VQYWKGERSLPNFKKDMAFPFGLQGISRLVYDEEKKAFC | |
| | | | FRLYRVPFKTYLGKDFTDKRMLLERLVKGDVKLCASNIQL | |
| | | | NGGKIFWLAVFEIEKEKHSLKPEVIAEASLSLEYPIVVKTG | |
| | | | KNRLTIGTKEEFLYRRLAIQAARRRTQVGATYSRSGKGKK | |
| | | | RKLKAVDKYHKTESNYVAHRIHVYSRKLIDFCIKHQAGTL | |
| | | | ILMNQEDKVGIAKEEEFVLRNWSYYELMTKIKYKAEKAG | |
| | | | IELIIG | |
| | | RpoE | MLYSDFELLKIGDTSALDHIHAKYFRSILWIGRQWLNDDF | 6045 |
| | | | LIESLVQDTFLKLWVNRDKLESPEHIFYFLRFVMKRECISY | |
| | | | YRKPKYKFHKKVNSLEDYDNYQDYMVGYDPVNDSENLD | |
| | | | EQESTQKSFDHIKSILPLLNADKRHLIELCLKYGFQYKAIS | |
| | | | KVMGKGIHETSREIKEAIEDIKTIVHKGNELGSNDTMTNEI | |
| | | | KFSGELSEEQAKVLKMRCDLKYSFSEIAKELELSEKEVHQ | |
| | | | EFMKAYKLMKANHQLQLQSA | |
| | | HTH | MLMADKNTNKSKVYFSDKYVCKFISEEWLTSKDTSARKY | 6062 |
| | | | GKIYGVNYHVIEKIQQENGYNIPLSTLSTICFNHGIKLSDFF | |
| | | | KLVEKKYGEFLNDSYEYK | |
| | | RpoA | MALLNFQKPDKVIMIDSTDFEGKFEFRPLEPGYGLTVGNA | 6080 |
| | | (RNAP) | LRRVLLSSLEGFAITSVRIDGVEHEFSVVPGVVEDVTEIILN | |
| | | | LKQVRFKRQIDDVESETVSISVSGKEQLTAGDFQKFISGYQ | |
| | | | VLNPDLVICNMGPKVSINMEIVIEKGRGYVPAEENKKSNA | |
| | | | PLGSIAVDSVYTPVKNVKYSIENYRVEQKTDYEKLVFEIIT | |
| | | | DGSIHPKDALTEAAKVLIHHFMLFSDERITLEADEIAQTET | |
| | | | YDEESLHMRQLLKTKLVDMDLSVRALNCLKAAEVDTLG | |
| | | | DLVSFNKNDLMKFRNFGKKSLTELEELVINKGLQFGMDLS | |
| | | | KYKLDKD | |
| | | RpoB | MFTNTIERVNFASAKNIPEYPDFLDIQIKSFQDFFQLETKSD | 6081 |
| | | (RNAP) | ERGNEGLYNTFMENFPITDTRNQFVLEFLDYFIDPPRYSIQE | |
| | | | CIERGLTYSVPLKARLKLYCTDPEHEDFETIVQDVYLGTIP | |
| | | | YMTPSGTFVINGAERVVVSQLHRSPGVFFGQSFHANGTKL | |
| | | | YSARVIPFKGSWIEFATDINGVMYAYIDRKKKLPVTTLFRAI | |
| | | | GFERDKDILEIFDLSEEVKVSKAGLKKVLGRKLAARVLNT | |
| | | | WHEDFVDEDTGEVVSIERNEIILDRDTILEKEHIDEIIDADV | |
| | | | KTILLHKENNAQSDYAIIHNTLQKDPTNSEKEAVEHIYRQL | |
| | | | RNAEPPDEETARGIIEKLFFSDQRYSLGEVGRYRMNKKLG | |
| | | | LDIGMDKEVLTKEDIITIIKYLIELINSKAEIDDIDHLSNRRV | |
| | | | RTVGEQLSQQFGVGLARMARTIRERMNVRDNEVFTPIDLI | |
| | | | NAKTLSSVINSFFGTNQLSQFMDQTNPLAEITHKRRLSALG | |
| | | | PGGLSRERAGFEVRDVHYTHYGRLCPIETPEGPNIGLISSLS | |
| | | | VFAKVNSMGFLETPYRKVVDGKVDVKEHIYLSAEEEEGM | |
| | | | KIAQANIPLKDDGTIDREKVIARDEGDFPVVDPVEINYTDV | |
| | | | APNQIASISASLIPFLEHDDANRALMGSNMMRQAVPLLRP | |
| | | | ESPIVGTGLERQVATDSRVLINAEGDGVVEYVDAQKITIKY | |
| | | | DRTEEERLVSFEEDSKTYELVKFRKTNQGTSINLKPIVRKG | |
| | | | DKVKKGQVLCEGYATEKGELALGRNMKVAFMPWKGYNF | |
| | | | EDAIVISEKVVREDIFTSVHIDEYALEVRDTKLGAEELTNDI | |
| | | | PNVSEEATRDLDEYGMIRIGAEVKPGDILIGKITPKGESDPT | |
| | | | PEEKLLRAIFGDKAGDVKDASLKASPSLRGVVIDKKLFSR | |
| | | | SIKDKRKRSEDKEAISRLEMDYEVKFQQLKDVLIEKLFGL | |
| | | | VNGKTSQGVINDLGEEVLPKGKKYTIKMLNAVDDFAHLV | |
| | | | GGSWTTDEDTNALVADLLHNYKIKLNDIQGNLRRDKFTIS | |
| | | | VGDELPAGIMKLAKVYIAKKRKLKVGDKMAGRHGNKGI | |
| | | | VARIVRQEDMPFLEDGTPVDIVLNPLGVPSRMNIGQIYETV | |
| | | | LGWAGLKLGQKYGTPIFDGATLDDINELTDKAGVPRFGHT | |
| | | | YLYDGGTGQRFDQAATVGVIYMLKLGHMVDDKMHARSI | |
| | | | GPYSLITQQPLGGKAQFGGQRFGEMEVWALEAYGASATL | |
| | | | REILTVKSDD VIGRAKTYESIVKGETMPEPGLPESFNVLMH | |
| | | | ELKGLGLDIRLEE | |
| | | RpoC | MARIKDNNAPKRFNKISIGLASPESILAESRGEVLKPETINY | 6082 |
| | | (RNAP) | RTHKPERDGLFCERIFGPVKDYECACGKYKRIRYRGIVCD | |
| | | | RCGVEVTEKKVRRDRVGHINLVVPVAHIWYFRSLPNKIGY | |
| | | | LLGLPSKKLDMIIYYERYVVIQPGIAKGPEGEEIHKLDFLTE | |
| | | | EEYLNILESLPSENQYLEENDPNKFIAKMGAECLIDLLARI | |
| | | | DLEQLSYELRHKANTETSKQRKTEALKRLQVVEALRESQ | |
| | | | DNRENNPEWMIMKVIPVIPPELRPLVPLDGGRFATSDLNDL | |
| | | | YRRVIIRNNRLKRLMEIKAPEVILRNEKRMLQEAVDSLFDN | |
| | | | TRKASAVKTESNRPLKSLSDSLKGKQGRFRQNLLGKRVD | |
| | | | YSARSVIVVGPEMKLYECGLPKDMAAELYKPFIIRKLIERG | |
| | | | IVKTVKSAKKIIDKKEPVVWDILENVLKGHPVLLNRAPTL | |
| | | | HRLGIQAFQPKLIEGKAIRLHPLACTAFNADFDGDQMAVH | |
| | | | LPLGPEAILEAQLLMLASQNILNPANGSPITVPSQDMVLGL | |
| | | | YYMTKEKRSTPEEPVIGEGLTFYSSEEVEIAFNERKVALNA | |
| | | | IIKVRTKDFNEAGELVNKIIETTVGRVLFNTVVPEQAGYINT | |
| | | | VLNKKSLRNIIGDILAVTDVPTTADFLDKIKTMGYEFAFKG | |
| | | | GLSFSLGDIIIPKEKHEMIAEANEQVDGIMMNYNMGLITFN | |
| | | | ERYNQVIDVWTSTNAMLTELAMKRIREDKQGFNSVYMM | |
| | | | LDSGARGSKEQIRQLTGMRGLMAKPKKSTAGGGEIIENPIL | |
| | | | SNFKEGLSILEYFISTHGARKGLADTALKTADAGYLTRRLV | |
| | | | DVSQDVIINTEDCGTLRGIEVEALKKNEEVVETLGERILGR | |
| | | | VSLHDVYNPLTEELILKAGQEISEADVKKVEAAPIEKVEVR | |
| | | | SPLTCEAAQGICAKCYGRNLATNKMVQRGEAVGVVAAQS | |
| | | | IGEPGTQLTLRTFHVGGIAGNISEDSKLEAKFDGIAEIEDLR | |
| | | | VVEGVDNGGGKSDIVISRTSEIKIVDAKTGITLSTNNIPYGS | |
| | | | QLFVKNGEKITKGTVICQWDPYNGVIVSEFTGQIAYENIEQ | |
| | | | GMTYQVEIDEQTGFQEKVISESRNKRLIPTLLIKDGKGETI | |
| | | | RSYNLPVGSHLMVDNGEKIKEGKILVKIPRKSAKAGDITG | |
| | | | GLPRVTELFEARNPSNPAVVTEIDGVVSFGKIKRGNREIIIES | |
| | | | KAGEVKKYLVKLSNQILVQENDYVRAGMALSDGSITPEDI | |
| | | | LAIKGPSAVQQYLVNEVQEVYRLQGVKINDKHFEVVVRQ | |
| | | | MMRKVQIQDSGDTTFLENQLVHKDDFINENDEIFGKKVV | |
| | | | EDAGDSERLKPGQIVTARQLRDENSILRREDKTLVTARDA | |
| | | | VAATATPILQGITRASLQTKSFISAASFQETTKVLNEAAVNG | |
| | | | KVDTLEGLKENVIVGHKIPAGTGMRDYDSIIVGSKEEYDEI | |
| | | | MARKEEFKF | |
| | | RpoZ | MQDLKNTKAPVSTATLNRNEFDSKTGNIYEAISIASKRAVQ | 6083 |
| | | (RNAP) | INSDIKKELLEKLEEFATYSDSLEEVFENKEQIEVSKFYEKL | |
| | | | PKPHALAVQEWLEDKIYYRNTEKDA | |
| Allomuricauda | Ata2 | dCas12f1 | MGKSTLKHTRKIQLLIDLPTKDEKKEVMDIMYQWRDRCF | 6029 |
| taeanensis* | | | RAANLIVTHLYVQEMIKEFSYLSEGIKYKLADEKKDEKGIL | |
| strain MCCC | | | NRSRINTTYRLVSDRFKGEVPTNILSTLNHGLISSFNKNRIQ | |
| 1K06752 and | | | YWKGERSLPNFKKDMAFPFGLQGISRIVYDEEKKAFCFRL | |
| Allomuricauda | | | YRVPFKTYLGKDFTDKRMLLERLVKGDVKLCASNIKLNG | |
| taeanensis* | | | GKIFWLAVFEIEKEKHSLKPEVIAEASLSLEYPIVVKTGKN | |
| strain MCCC | | | RLTIGTKEEFLYRRLAIQAARRRTQVGATYSRSGKGKKRK | |
| 1K06699 | | | LKAVDKYHKTESNYVAHRIHVYSRKLIDFCIKHQAGTLIL | |
| | | | MNQEDKVGIAKEEEFVLRNWSYYELMTKIKYKAKKAGIE | |
| | | | LITG | |
| | | RpoEl | MSYSDFELLKIGDSSALDHIHAKYFRSIFWIGKQWLNDEFL | 6046 |
| | | | IESLVQDTFLKLWVNRDKLESPEHIFYFLRFVMKRECISYY | |
| | | | RKPKYKFYKKVNSLEDYENYQDYMAGYDPVNDSKNLDD | |
| | | | QESTQKSFDHIKSIFPLLNADKRHLIELCLKYGFQYKAISK | |
| | | | VMGKGIIETSREIKEAIEHIKTIVHQGNKLDSSDTMTNQIKF | |
| | | | SKELSEEQAKVLKMRCELNYTFSEIAKELELSQKEVHQEF | |
| | | | MKAYRLMKANHQLQLQSA | |
| | | HTH1 | MAKKNTNKSKVYFSDKYVCRFISEEWLTSKDTSARKYGK | 6063 |
| | | | IYGVNYHVIEKIQQENGYNIPLSTLSTICFNHGIKLSDFFKL | |
| | | | VEKKYGEFLNDSYEYK | |
| Allomuricauda | Ata3 | dCas12f2 | MEKSTLKLTRKIQILIDLLTKEEKKEALDKLYQWQNRCFR | 6030 |
| taeanensis* | | | AANLIVTHLYVQEMIKEFFYLSEGIKYKLADEKKDEQGIL | |
| strain MCCC | | | NRSRINTTYRVVSDRFKGEIPTNILSNLNQALISSFKKNRSE | |
| 1K06752 and | | | YWNGERSLKNFRRDMAFPFDLEGMSGLAYNEEKKAFCFR | |
| Allomuricauda | | | LFRIPFKTYLGKDFTDKRTLLERVVQGKTKLCTSHIKLKDG | |
| taeanensis* | | | KIFLLAVFEIEKERNDLRPEIIAEASLSLEYPIVVKVGKARLT | |
| strain MCCC | | | IGTKEEFLYRRLAIQSAHRRAKIGATYSKSGKGIKRKLKAV | |
| 1K06699 | | | DRLGQAERKYVHNRLHVYSRRLIDFCVKHRAGTLILLNQ | |
| | | | EEKTGIAKEEGFVLRNWSYNDLMTKIKYKANKAGLEVIID | |
| | | RpoE2 | MENQNSLEECYERLKKGCAISFTEIYTKYHRQIFWLGKSF | 6047 |
| | | | LDDGFVVETLVQDVFLKLWVNRDSLESPKHIYFFLRFVMK | |
| | | | RECITYYTRPRNKFFRKVHSLESFENYQDYMVGYDPAVDN | |
| | | | NNVKLQEGEQEKFESIKRVLPLLDDSKRHIINLCLKYGFQY | |
| | | | KAISKVMGKGINETCREIKEVIEDIKTILHRGNKLDSSNNN | |
| | | | MDEIKFTGEMTEEQTKVLKMRCELRYSFSEIANELNLSQK | |
| | | | EVHQEFMIAYRLMEAKHQLQSA | |
| | | HTH2 | MPTDTKANHIGKKIARIRELRGMKQETLAEELGISQQSVST | 6064 |
| | | | LEKSETLEDKKLEEIAKALGVTKEGIENFSEESVLNIISNSF | |
| | | | HDQSALNAILNQPTFNPIDKVVELYERLVQAEKDKVTYLE | |
| | | | KLLDKK | |
| Allomuricauda | Aru | dCas12f | MEKTTLKLTRKIQLLIDLSTKEEKKEALDKLYQWQNRCFR | 6031 |
| ruestringensis | | | AANLIVTHLYVQEMIREFFYLSEGKKYKLADEKKDEQGIL | |
| | | | NRSRINTTYRVVSDRFKGEIPTNILSNLNQALISSFKKNRPE | |
| | | | YWNGERSLKNFRRDMAFPFDLEGMSGLHYNEEKKAFCFR | |
| | | | LFRIPFKTYLGKDFTDKRTLLERVVEGKTKLCTSHIKLKEG | |
| | | | KIFLLAVFEIEKENHDLRPEIIAEASLSLEYPIVVKVGKARLT | |
| | | | IGTKEEFLYRRLAIQSAHRRAKIGATYSKSGKGIKRKLKAV | |
| | | | DRLGQTERKYVHHRLHVYSRRLIDFCVKHRAGTLILLNQE | |
| | | | EKTEIAKEEGFVLRNWSYCDLMTKIEYKAKKAGLELIID | |
| | | RpoE | MENHHTLEECYKGLKKGCSNSFTEIHTKYNRQIFWLGKSF | 6048 |
| | | | LDDGFVVETLVQDVFLKLWVHRDTLESPKHIYFFLRFVMK | |
| | | | RECISYYTRPKNRFFRKVHSLESFENYQDYMVGYDPAEDN | |
| | | | NNVKLQEGEQEKFESIKSVLPLLDDSKRHVINLCLKYGFQ | |
| | | | YKAISKAMGKGINETCREIKEAIEDIKTILHQGNKLDFGDN | |
| | | | NTDEIKFTGEMTEEQTKVLKMRCELRYSFSEIADELNISQK | |
| | | | EVHQEFMIAYRLMEAKHQLQSA | |
| | | HTH | MTTDTKTNHIGRKIARIRELRGMKQETLAEELGISQQSVSS | 6065 |
| | | | LEKSETLEDKKLEEIAKALGVTKEGIENFSEESVLNIISNSF | |
| | | | HDQSALNAILHQPTFNPIDKVVELYERLVQAEKDKVSYLE | |
| | | | ELLKKK | |
| Salegentibacter | Smi | dCas12f | MGKDTITLTRSIRLEIDLPTQEERQEAKSKLYQWRYRCHK | 6032 |
| mishustinae | | | AANLIVSHLYVQEMIQEFFYLSEGVKYKLVDEKKDELGIF | |
| | | | NRSRMNTTYRLVSDRYKGKMPTNILSQLNSIIQSSFKKNRE | |
| | | | EYWKGERSLQNFKKEMAFPFTMEGVCGLEFNPEKSAFCF | |
| | | | RFFSIPVKTYIGRAFNDKWKLMHQLTKGEIKMRTSYLKLK | |
| | | | DGKIFMMAAFEMEKEKHQLRPEVFAEARLSLEYPIIVKIG | |
| | | | KAKLSIGSREEFLYRRLAIQAARRRTREGVKYARSGNGHK | |
| | | | RKTKAAARFKDKERNYVNQRLHVYSRELIDFCVKHQAGT | |
| | | | LILVDQEQKIELAKEEAFVLRNWGYYDLMTKIKYKAEKA | |
| | | | GIELIIG | |
| | | RpoE | MLQRIFELLQQGHPDALEFIHTKYHRNIFWVGKQILDDDF | 6049 |
| | | | AVETLVQDTFLILWEKRDRIERPEHIYYFLRMVMKRECYT | |
| | | | YYVRPKNKFFKTVNSLESFENYQEYLHGYDPEKDDLHLL | |
| | | | NHEIQQKAFDRISRVLPLLSPERRRLIELCLKYDFRYKAIGQ | |
| | | | LMGTSITHTSNEVKKAIVDIKNIICQRSIQETKPKPVLAVKI | |
| | | | QREMTQEQEKVLQLRTERQYSFAAIAKELNLSEKEVHQEF | |
| | | | MSAYKLMQLKHEQQQSA | |
| | | HTH | MIILVVDRATMQNKNYKEDFLLKFGENFGKIRRSKSLSFRS | 6066 |
| | | | LSQKCDIDYADLNKIERGKRNITLTTIIELARGLDIHARELF | |
| | | | DFSFTLKDLEK | |
| Leeuwenhoekiella | Lpa | dCas12f | MMKNKTLSLTRKIQLKVDLPTYEERKEAIGKLYQWQNRC | 6033 |
| palythoae | | | FKAANIIVTHLYCQEMIKEFFYLTEDIQYKLADQKKDENGI | |
| | | | LNRSRINTTYRVIADRFKGEIPMEILSNLNRNLESSFNKNKP | |
| | | | KYWKGERSLKNFRRDIGFPFPARCMWGFKHDPERNAFCF | |
| | | | RLFQIPFCTYLGRDFSDKRSLLHRAVKGEVRLRTSEIKLTD | |
| | | | TKIFWLAVFDIEQEQHALKPEVIAEASLSLEHPISVKVGGN | |
| | | | RLNIGNKEEFLYRRLAIQAARKRALAGTVNSRGGHGRTRK | |
| | | | LKAVEKYKDKETKYVNQRLHVYSRRLIDFCVKHQAGTLI | |
| | | | LLHQEDKIEAAKENQFVLRNWNYYELTKKIEYKAKKAGI | |
| | | | ELVID | |
| | | RpoE | MKRTAADKHYTGFKHGCPVALKAIYTQYHRQIYWMGRS | 6050 |
| | | | LIKDVFVVETLVQDTFLTLWEKRESIESPQHLVNFLYTVISN | |
| | | | ECKWYYARPKNQFNRECYALEKMENYQSFMLGYDPTAV | |
| | | | DIHLEDQQQQQREYEQVIKVLPLLGGQRQRLIELCLQHGF | |
| | | | KYKIISEQLGISIKEASTTLKLTIDEIKNILHQGYVLQPQETE | |
| | | | PMQNGEGQMTEQEARVLALRCEQQYSFAQIANELQISQK | |
| | | | EVHRAFTAAYKLLQQHEHLQSA | |
| | | HTH | MLRFFTFIANYQFDMNEDQKRRLLIEFGKIVKFHRTEKLKI | 6067 |
| | | | SLRDLAKKCDVEHSAIGKIEKGEIKIQLPTVFELAKGLEIHP | |
| | | | RDLFDFDFPLEETGN | |
| Sphingobacterium | Sda | dCas12f | MEKESIILTRKVQIYLDCDDKGQRSAYFKQLFEWQDMVY | 6034 |
| daejeonense | | | RGANMVMTHQFVQEQIKDLIYLRDDVKVKLADFKKDPE | |
| | | | GIFNSSKMNTTYRILSLYFKGKLPSKIISAMNMTLNRAYST | |
| | | | DRSSYWKGEKSLRNYRKDMPIPFGGDQLKLGNDEKGRDF | |
| | | | RFTLFKIPFRTYLGKDRSDKRILLQRCLVGQIKICTSSIKMV | |
| | | | KGKIFLLLALELPKKQHDLKEHIIAEASLSVEHPITVSIDRD | |
| | | | NFQIGNKEEFLYRRLAIQAARHRIQKAVAFNRGGHGRRKK | |
| | | | LKSLEHFTEREKRYVDSKLHLYSRRLIDVCVNSGAGTLLL | |
| | | | VNQSNKEEAAKEDRFLLRNWGYYGLIEKIRYKANMAGIN | |
| | | | VIVE | |
| | | RpoE | MNETLRQQETDRHIALLRKGDEKGLNFFYRRFYGYIFARA | 6051 |
| | | | FRATQDDCAAKSIAQEALLRLWLFRKQLKDAEDIQAFLKA | |
| | | | QVRSSINAYYNKTRNRFHRSLLRLDSIEDYQEFLLGHEME | |
| | | | EEEEMDIVYLERLDQEKQQRLIQLDNLMPSLNGQQQMFIK | |
| | | | LCLKYSFNYERIAYYLGGISDYQVSLQVEKNDRYPTFYIQ | |
| | | HTH1 | MNAIFREKNENMHIGHNIKRIREIQGIKQEAFGQLCRNRYS | 6068 |
| | | | QQRISDFENMVALDEPLLNELASALGVTPEFVKSFKEENVI | |
| | | | YNIQHSHTFNDHSTNSSQHTQPTFNSDGSDKLVALLERFIE | |
| | | | EDRAKTRSIAELSKAVLDLTNEVKKIKEGK | |
| | | HTH2 | MEQMNRSSKIVAQGELDEQQAEIFHMRYQLQLSFDQISEA | 6069 |
| | | | LHLDPSTVRKIFVQAHTKIRTAQRT | |
| Leadbetterella | Lby | dCas12f | MEKIILTRKIQLVIPCKDREILKSYYDRLYEFQRHTCKAANL | 6035 |
| byssophila | | | IHTHLFIQDRWKEMVYLAEDAKISLADHKKKEGGVLNTS | |
| | | | RMNSTYRQLSAHFLKVLPSNTMSNLNQAVYRTYQANRDL | |
| | | | YWRGEKSLPNYRRDIPIPFSSREMRWEEAGDGKNFLLYLF | |
| | | | RIPFKTYLGRDRSELKSLLKKIVKGEIALRQSALQIKNNKIY | |
| | | | LLASVEVEKAKHSLDKTLIAEVALSLEYPLVIKIGKDEFQIG | |
| | | | SKEEFLYRRLSIQAARRRLQQACAYRSGGKGKEERYKCLK | |
| | | | HYKEKEKNYIEEKLHLYSRRLIDYCVKAGAGTILLVNQSY | |
| | | | KEELAKEDPMLLRNWSYYGLKEKIAYKAKMAGIHVLVE | |
| | | RpoE | MNIENINLIKLKNGNEAGLSYFYRRFFPWYTFRAFRYMRD | 6052 |
| | | | DLDAQCAVQEAFLRLWLNRAQVDSVESMHEFLKRQVQE | |
| | | | AAKAFHRKRSNEFRKSMLYYFDYDDPDILIGHRAVEEEVT | |
| | | | EELQPDASDQEKLDSIHRLLPHLGREQELFIRLCLRFNFNY | |
| | | | ERIAFYLGGIRDYEVANKVNKCIVQLKTLVADSSKLSSASK | |
| | | | METIRVDERLTPEQAEVLKLRYELGASFDDIGQALQMPVS | |
| | | | RVREIFIQAFTVFKHGKNHSYSKNTVSYSL | |
| | | HTH1 | MTKKKTPFGKYLESKSINQAALARTTGIRPNRISEFATQDC | 6070 |
| | | | LKMRADEIYLLAKALGERPGTLLDYLLAEIKS | |
| | | HTH2 | MEPKIHEGKNVKRFREMLGIKQEALAQELGEDWTQKKVS | 6071 |
| | | | LMEQKAVLEPEVLYKVSKALNIPDMAIKNFDEEKAITIIAN | |
| | | | TVNNNDHATGNSLFNYQPIFNPIDKIVQLHEEKIQLYERML | |
| | | | REKEDMINKLEKLLPQN | |
| Mucilaginibacter | Mri | dCas12f | MADNMYITRKIQIIVNSPDKDVVYDAIGKLMQWQQACYK | 6036 |
| rigui | | | CANLIYTHQFLQEQIAEMVYLADGVKLKISDHHKDADGM | |
| | | | LVSSRTNSTYKVLTEKFKGVLPSSIYNNLNSQLVSTFLKER | |
| | | | GLYVNGERSIRNFKRSIAMPFSAENIRRLTAGDHGNFTFILF | |
| | | | GIPFRTYLGRGYDEKRELLRQVVNGKIKLASSFLKVEQKK | |
| | | | VFLLATFEQEKQFHLLGGAIIAEASLSLEYPLTVKVGKARM | |
| | | | TIGSKEEFLHRRMAIQAARSRVQASVDGNKAGHGKVRKR | |
| | | | KPLAHYQSLEKDYIKHKLNVYSKRLIDFCLQHRAATLILTG | |
| | | | QQEKEEIAMAEPFLLRNWNFAGLKEMIAFKAAKVGIDLIV | |
| | | | E | |
| | | RpoE | MVGKLCEISSFDDKVFEKILKQYRLTIYSFGKRMLNDSYIV | 6053 |
| | | | ENIVQDAFLKLWNFRQTITSDEHARRFLMQSVKWACYSY | |
| | | | FRNSDSRFHRNMIRLNDYDNPADLFGEHPDVQTYNDHTE | |
| | | | ALNESRLNEVKEAIDKVCYGREKEVMELHFIKGLSHSQIA | |
| | | | ERYHLSIRTVTVIIEKGTVRLKTILVTVKTPVHDIQLSPAPGF | |
| | | | VDSFNHIEGLNEEQNKIYHLRLTGRYDFEQIASFLQLPLAF | |
| | | | VQTEYLKAWKIAACLKKKNGKPAGRANKGISGRYGLLSA | |
| | | HTH | MTNLGLFLAKKSVNKAEISRKTGISKSRLSELSMNNSTKL | 6072 |
| | | | RADELYLIALAIDVDPKELLNHLFMDLKLKD | |
| Puia dinghuensis | Pdi | dCas12f | MNAETMILTRKVQLIIDSNDKAFIGEVYRTLYRWQYICFRA | 6037 |
| | | | ANYIFTHLFIQEQLKELFYLKDEVKVKLADCCKNPDGILTC | |
| | | | SQLGTTYRVLNKHFKGDIPMNIISSLNMTLAKHENNEKEG | |
| | | | YLKGEKSVRNYKRDIPIPFQRRNITRLQLAENAKEYKFNLF | |
| | | | KIPFHTYLGRDKFDKRLLFDRLLKGEVQLKNSSLQLCNGK | |
| | | | IYLLAAFETRKEKHELDASIVAEAHLSIDYPIVVRIGKFQAT | |
| | | | IGSKEEFLYRRLAIQAARSRAQKDASYNRGQHGRRRKLK | |
| | | | ALEHFRDRERDYVQQRLHVYSRQLIDLCVKHRAASLILVG | |
| | | | QTEKEAAAAGEEFVFRNWSYYALKEKIQYKANKAGIMLI | |
| | | | TE | |
| | | RpoE | MSYELPTPTEQAYFLRYKNGEEEGFTQLYRMMFNSLLRYG | 6054 |
| | | | MRILPNEFAVTTIVQDALLKAWDFRERMTCLQHTFRFMC | |
| | | | MNVKWACYDYYRQPEIRQVVYLDHDTYPDVSFLPGSEEA | |
| | | | GPVCNEEALLKSIYDVMPYLPMNKQTILQLYFKYGFSYKQ | |
| | | | IAKRYGANIQTISKNLHEALAYLKKVIHSKKQLTKPISFPVT | |
| | | | NDKYQAEEYLTGEMLQLFKLRYESKLPFDVIAAKLNLPQP | |
| | | | YIQQQYAAAHAKLQQLKISRRP | |
| | | HTH1 | MKTKIDLYVITRVKEKRLEKNISQAELANELGMSVGFIGK | 6073 |
| | | | VESPKYPSHYNIKHLNQLAKILDCSPQEFLPKKPLP | |
| | | HTH2 | MKSKIDIYVIDKVREMRIAHNMSQEELSIKAGFRSNGFVG | 6074 |
| | | | QAESFKYNKRYNVHHINRFAQIFNCSPQDFLPETYLY | |
| Pedobacter | Psu | dCas12f | MESNKMVITRKIQLLIDSEDKEEVKKMKDQLYNWQWITY | 6038 |
| suwonensis | | | RSANMIMSHHFVQEQVKDFFYLTEDIKLKIADEKKEENGI | |
| | | | LKSSCQNTTYRLLSNHFKGQIPTNILSNLNNTLISYFNKEKS | |
| | | | AYWKGEKSLRNYKKNIPMPFEASVISKFVYTPDRRNFSFK | |
| | | | LFKIPFRTYLGKDRSDKKIMLEKIMNGTLKLCVSNIQLDKG | |
| | | | KIFLLAAIQVDKEQHTLDTSIIAEASLSIEHPITVKIGKYEHT | |
| | | | IGTKEEFLHRRLAIQAAIYRVQKAVKFNRGGHGSKRKRRS | |
| | | | LVDYQHQEKRYVEYKLHLYSRMLINLCLKYQAATLLLLN | |
| | | | QEEKEEIAKDDVFLLQNWSYYSLKEKIAYKAARAGIQMIV | |
| | | | E | |
| | | RpoE | MNNNYKIAMQTIIKKQNQDVNFARFKEGDEKGLEFFYKR | 6055 |
| | | | LYPALYFYSFRYIKDDINADCIVNEAFLRLWLVRRSIQDPD | |
| | | | HIEPFIKKLTTQACKAYYRTSNKRFQRNMLRLDEIENYDEF | |
| | | | IFGHDPEIEEDTEVICQEELENELKEKWIRLKTLIPNLTQDQ | |
| | | | QLIVRLCLKYSFNYDRIAWHIGGISDYQVARKVEKTLESLK | |
| | | | AIFTNSQKLEIVGNNNRFRFEGDLNEEQSSILHMRYQLQYS | |
| | | | FEEISSALNLDQGYIKKVFVGASIKIKKVKM | |
| | | HTH | METKEQFKKTHLGRKISRIREIRGIKQDALAMELGLSQQTI | 6075 |
| | | | SKIEQSEDVDDETLNKISKALGVSSDAIKNFNEEAVVNIIA | |
| | | | NTVNNHDQSASVFISPNFNPIDKIVELYERLLKSEQEKNEL | |
| | | | LNKK | |
| Chryseobacterium | Cgl | dCas12f | MEKSTMTLTRKIQLMIDLPSDKKNEMWEKLYRYQNLCFR | 6039 |
| gleum | | | AANLIASHLYVQEMIKDFFYLTEEIQYKLADEKKDEMGMF | |
| | | | NRSKTGTTARMVFDRFKGEIPTDILGSLNNTIQSTFSKNKA | |
| | | | DYWQGTKSVRNYKRDIPIPLPVKCISKMKYDPDKKAFCFN | |
| | | | MFAIPVKTYLGKDYTDKRVTMERLLKGDIKLCTSQIQLKD | |
| | | | RKIFWLAVFEFKKEENHLKPEIIAEASLSLEHPIVAKANNLR | |
| | | | INIGSKEEFLYRRLAIQASQKRIQDGIAYARSGNGSKRKQK | |
| | | | ALYKTENLESRYVTHRLHMYSRKLIDFCVQQQAGTLILKN | |
| | | | QEDKIGIAKEQEFVLRNWNYYELQTKIKYKAEKAGIELIIG | |
| | | RpoE | MKRTNSPPLKLTDFQLYKLLKKGNPSSLEHIHLRYKRLLF | 6056 |
| | | | WIGKQMLEDDFAVETLVQDTFLKLWLHRDSIETPNHILGF | |
| | | | LRFVLKRDCITYFNTPKNKFARLTASLESFENYQDYIVGYD | |
| | | | PVQDKEHLLRQESDQKNFDEVNKVLKVISPKRKYLIELCL | |
| | | | QYGFQYKPIAEAMGSSVKDISNEVTRAINDLRKILRENSN | |
| | | | DEPPIKSKKNEVKQNELSGQQIEIIKRRFREKSSFAIIARELK | |
| | | | LSEKEVHQDFLYAYQYLQNQNNSEITI | |
| | | HTH | MNESLEEIERYVIKRVKEIRESKNVTQEELSLSIGKNIGFISQ | 6076 |
| | | | IEAPSKKAKYNLIHLNLIAIALGCSIKDFLPDEPIRDKKYDI | |
| | | | KEIQNKKS | |
| Zunongwangia | Zpr | dCas12f | MGKETIKLTRKIQLLVDAPNKEERKEALDTLYRWQNRSYR | 6040 |
| profunda | | | AGNLIVTHQYIQEMIKDFFYLSEGIRYRLVDEKKAEDGILN | |
| | | | RSKSNCTYRVVSDRFKGEVPTNILANMNYNIMNNFSKNLV | |
| | | | QYRRGERSLANFRRDIPFPFGTIGIHGLSYKKEKKAFCFRL | |
| | | | FSIPFKTYLGKDYTDKRSLLEQVVAGNIKLCTSKIQLNKGK | |
| | | | IYWLAVFEVAKEKHNLKPEVIAEASLSLEHPIIVKSRKATLS | |
| | | | IGSREEFLYRRLAIQAALKRAQNATAYCRSGKGRKRKTKA | |
| | | | VERFHEKEKNYVSNRLHVYSRKLIDFCIKHEAGTLILLNQE | |
| | | | DKMEIAKEDGFVLRNWNYYELMTKIKYKAEKAGIELIVD | |
| | | RpoE | MEREFKLLKEGHPDAMEFIYARYQHKLFWMGKQLIKDEF | 6057 |
| | | | VIESILQDTFLKLWEKRDHIEDPKHMLYFLLHVMKRDCSY | |
| | | | YYIRPRNNFHRNINSLDNYENYQEYIHGYDPESEDEHLKD | |
| | | | QEANQKALDRIKCVFPLLRPERRYLIELCLKYGFQYKNIAE | |
| | | | LMGTSTTYTSNEVKRAIDDIKKIIHQGSNLGSKPDQIQVKK | |
| | | | NTRITREQEKVLQLRNEMHYSFAAIAEELQLSQKEVHKEF | |
| | | | MTAYKLLQSKHKQQQSA | |
| | | HTH | MQNEKDKVDFLIQFGSNFGKIRKMKNLSFRALSQKCDLD | 6077 |
| | | | YADLNKIEKGKRNITLTTIAELARGLNVHPKELFDFDFTP | |
| Chryseobacterium | Cba | dCas12f | MEKSTMTLTRRIQLLIDLPANEQKEMWEKLYRYQNRCFRA | 6041 |
| balustinum | | | ANFIVSHLYVQEMIKDFFYLTEDIQYKLADENKDKMGIFT | |
| | | | RSKTHTTARMVFDRFKGEIPTDILGSLNNTIQSTFSKTKAD | |
| | | | YWQGTKSLRNFKKDIPIPLPVKCISKMKYDPEKKAYSFNM | |
| | | | FAIPVKTYLGNDYSDKRVIMERLLREEIKLCTSQIQLKAGK | |
| | | | IYWLAVFEFEKEEHKLKPEIIAEASLSLEHPIVVKANNVRIN | |
| | | | IGSKEEFLYRRLAIQASQKRIQDGIAYTRSGNGVKRKQKAL | |
| | | | YKTENLESRYVSHRLHLYSRKLIDFCIQQQAGTLILKNQED | |
| | | | KIGIAREQEFVLRNWSYYELQTKIKYKAEKAGIELIIG | |
| | | RpoE | MKRTNSLPRKLTNLQLYELLKKSNPTALEHLHLRYKRLLF | 6058 |
| | | | WVGWQVLKDDFVVDTIVQDTFLKLWLHRDTIETPDHITG | |
| | | | FLRFVMKRDCISYVTAPRNKFNRLMASLDSFENYQDYLA | |
| | | | GYDPLKDKEYLLSQESDQKNFDEVKKVLPVLDPKRKHLIE | |
| | | | LCLEYGFQHKPIAEAMGSSVKDISNEVSRAINDLRKILNRS | |
| | | | SSEQPKGKALDNKKQSEKLSSQQLDILKRRFEQKSSFAVIA | |
| | | | QELKLPEKEVHREFLYAYQHLQNQNTSEIPL | |
| | | HTH | MDVLKDEILKKFGEHVKDLRIKSGLTQDEVVLNSSKITKG | 6078 |
| | | | TVSDIENGKRNFAFTTLIDLAKGLNVSPKDLLNFKID | |
| Paenimyroides | Pba | dCas12f | MEKTTMKLTHKFRIVVDLPTYAERKEAMDKLYRWRNRC | 6042 |
| baculatum | | | YRAANLIVSHLYVQEMLKEFFYLTEGVQYKLADEKKHEA | |
| | | | GMLTRSRINTTYRALSDRFKGEMPMNILSCLNNSIISSFRK | |
| | | | ESEAYCKGERSIKNFKKTMAFPFGLEGIGGFCYNEEKRTFY | |
| | | | FRLFSIPFRIYLGKGRTEKTKVLQQVISGEIKMCSSHIKIND | |
| | | | GKVFWLPVFEIKKEEPTLKPEIIAEASMSFEYPLIVKIGRAR | |
| | | | YTIGTQEEFLYRRLAIQASLERLRVGAQYCRSDKGTRRKL | |
| | | | KATEKLKKAESNYVNNRLHVYSKRLIDLCVEHKAGTLILA | |
| | | | DQQEKMEVAKTEEFVLRNWSYYNLMTKIKYKANKAGIEL | |
| | | | IM | |
| | | RpoE | MPERNFELLKNSDPAALEKIHAQYRRLIFWVGRRWIDDDF | 6059 |
| | | | VVENLVQDTFLKLWECRETIKDPLHILFFLKFVMKRNCYA | |
| | | | HHAKPRNKFFKTNVHSFESYENYENSVTGYDPADAVQDL | |
| | | | KGQEEDQQFFDHLNTVLPLIRPERRHLINLCLKYGFRYKAI | |
| | | | AQVMGKGIMETVNEVKRAIEDIKVIVDRRKVLEKKDTGM | |
| | | | VETVPQTISERQSQVLMLRCEKKFSFAAIAQELNLSQKEV | |
| | | | HAEFMAAYKFRQQNMKELL | |
| | | HTH | MKKIIDFETTQFDYDLINHIKGLRKIHSITKEELSVKMGVA | 6079 |
| | | | KSFVGNVESATQRHKYATRHLTLLAKALGFKNISDLLKFPT | |
| | | | PEYDKIKVTVEQTYNEAGTK VIKSEVVKIEPIE | |

*also described as Flagellimonas taeanensis or Muricauda taeanensis

TABLE 8

| | | | | TAM |
|---|---|---|---|---|
| | gRNA sequence: | | | determined |
| gRNA | scaffold + native guide | 20 nt native guide | | by MEME- |
| Description | (RIP-seq footprint) | sequence | Original gRNA region | ChIP (5′-3′) |
| Pum_ | CAATTAAAAACTCA | CAATTAAAAACTCAC | TAAACAATATATTAAGGATAC | GGTT |
| dCas12f | CCCTAAAGCAAAGG | CCTAAAGCAAAGGA | ATTTACACCCGTAAGGTGAGG | |
| native | AAGTACAATTAATA | AGTACAATTAATAGCT | TTGTTGGTACATACTCACGGA | |
| | GCTAGTTTAATTGTA | AGTTTAATTGTAATCA | ATGCACAAAGCATATACAGCT | |
| | ATCAATAACAAAGC | ATAACAAAGCACAGC | AGAAATATTAATTAAAAAAAA | |
| | ACAGCAGATTACTG | AGATTACTGATATGAT | GGCTAACCTTAAAGAAAAAG | |
| | ATATGATTATTGGAA | TATTGGAAAAATGAA | AAGTACAATTAATAACTAGTT | |
| | AAATGAAACTGTTG | ACTGTTGTGAATAAT | TAATCGTGATCAATAACAAAG | |
| | TGAATAATAATATTT | AATATTTGAGGGTGC | CACAGCAGATTACTGATATGA | |
| | GAGGGTGCCTGTAC | CTGTACACCCGTAAG | TAATTGGAAAAATGAAACTGT | |
| | ACCCGTAAGGTGAG | GTGAGGTGGTTGATA | TGTGAATAATAATATTTGAGG | |
| | GTGGTTGATACATA | CATACTCACTGAATG | GTGCCTGTACACCCGTAAGGT | |
| | CTCACTGAATGCAT | CATAAGGCATATACA | GAGGTGGTTGATACATACTCA | |
| | AAGGCATATACAGC | GCAAAGATAACAATT | CTGAATGCATAAGGCATATAC | |
| | AAAGATAACAATT | AAAAACT (SEQ ID | AACAAATATAACAATTAAAAA | |
| | (SEQ ID NO: 6084) | NO: 6094) | CTCACCCTAAAGCAAAGGAA | |
| | | | GTACAATTAATAGCTAGTTTA | |
| | | | ATTGTAATCAATAACAAAGCA | |
| | | | CAGCAGATTACTGATATGATT | |
| | | | ATTGGAAAAATGAAACTGTT | |
| | | | GTGAATAATAATATTTGAGGG | |
| | | | TGCCTGTACACCCGTAAGGTG | |
| | | | AGGTGGTTGATACATACTCAC | |
| | | | TGAATGCATAAGGCATATACA | |
| | | | ACAAATATAACAATTAAAAAC | |
| | | | TCACCCTAAAGCAAAGGAAG | |
| | | | TACAATTAATAGCTAGTTTAAT | |
| | | | TGTAATCAATAACAAAGCACA | |
| | | | GCAGATTACTGATATGATTATT | |
| | | | GGAAAAATGAAACTGTTGTG | |
| | | | AATAATAATATTTGAGGGTGC | |
| | | | CTGTACACCCGTAAGGTGAG | |
| | | | GTGGTTGATACATACTCACTG | |
| | | | AATGCATAAGGCATATACAGC | |
| | | | AAAGATAACAATTAAAAACTC | |
| | | | ACCCTAAAGCGAAGGAAGTA | |
| | | | CAATTAATAGCTAGTTTAATTG | |
| | | | TAATCAATAACAAAGCACAGC | |
| | | | AGATTACTGAAGTGATTCTTG | |
| | | | GACAAAAGCAGAACCTGTTG | |
| | | | TGAGTAATAATATTAGAGGGT | |
| | | | GCCTATACACCCGTAAGGTGA | |
| | | | GGTTGTTGGCAAACAATCAAT | |
| | | | GAATGCGTACGGCAGATATAA | |
| | | | CAGAGAATATTTAGAGTTTAT | |
| | | | TAATGGAAAGCTTTTGTCAAG | |
| | | | TATATGTAGCTCCGTAGTGGTT | |
| | | | CAAAACAACAATAGTTAGTGT | |
| | | | ATACTTAATTACGAAGCTATAT | |
| | | | TGAAATACAGTATGTATAGGG | |
| | | | ATATAATATTTTGAGAGTGGTT | |
| | | | GTACACCTATATAGTGAGGTT | |
| | | | GCGGGTAAAAAGTCACTGAA | |
| | | | TGCAGTAAGAATATAAAACTC | |
| | | | TGAAAAAACTTATAAAAATAA | |
| | | | ATAAGAACCTTTAAAAGCCTT | |
| | | | ACAAATACATTTTTCACAGAA | |
| | | | ATAGTTAAAGTGTTTAAAAAT | |
| | | | AGTTAATTATAACGTTCTGCG | |
| | | | TTTTCTAAATTAATAATTATTAT | |
| | | | TATTTTTGAAATAACTTACCA | |
| | | | ACATAATTATTAA (SEQ ID NO: | |
| | | | 6104) | |
| Ata_ | ATTTTGAGGGTGCT | CAATTAGATTTTGAG | CAATTAGATTTTGAGGGTGCT | G |
| dCas12f | TGTACACCCATAGG | GGTGCTTGTACACCC | TGTACACCCATAGGGTGAGGT | |
| native | GTGAGGTTAAGAAT | ATAGGGTGAGGTTAA | TAAGAATTACACTCACTAAGT | |
| | TACACTCACTAAGT | GAATTACACTCACTA | GTGAACAACACATACAACTT | |
| | GTGAACAACACATA | AGTGTGAACAACAC | GTGGGATATACGCTAACAATA | |
| | CAACTTGTGGGATA | ATACAACTTGTGGGA | GCAATCAATAAGCCTAAATAC | |
| | TACG (SEQ ID NO: | TATACGCTAACA (SEQ | GGGCACCTAAATACAATATGG | |
| | 6085) | ID NO: 6095) | GACAAACACCGATAAAGTTT | |
| | | | TTTTGAACGATTGAAAATGGA | |
| | | | TACTTTTAATGAAGTGTCCAG | |
| | | | AAATCGTCAAATATTAGATAG | |
| | | | AACCCTTGGGATATTGCTTCT | |
| | | | CCTTATAGGTATCGCAATTGG | |
| | | | GATTTTCCTTATTCCTGATTTT | |
| | | | TTACCACTACTTAAAAGGACT | |
| | | | CCTTATTTATTGCTCGAACCC | |
| | | | GATGCAGGTGTAAGACATGA | |
| | | | GCTTGGATACGCTTGGTTTAT | |
| | | | GCAATCAATTGGTTGGATCAT | |
| | | | TGCTTTTTTCGCATTTAGGAG | |
| | | | CAGTAGGGCATTTTTAAGGGA | |
| | | | TAGCAAGAAACCAACTAGTA | |
| | | | CCAGTAATTAAATCAAAGGTG | |
| | | | AAAGAGGTATATAGTTGCCTA | |
| | | | AACACCATATACAACTCACTG | |
| | | | AAAAACCGCTCCGAAACGCA | |
| | | | ATATTCTGTGATGACAGCCAT | |
| | | | TCATCCGGTAGGCTAAAGTAT | |
| | | | GTAAAAAGAAGCTAATGTCAT | |
| | | | CTTCCCTTTT (SEQ ID NO: | |
| | | | 6105) | |
| Smi_ | TTTTCGAGGGTGCT | AATATTTTCGAGGGT | AATATTTTCGAGGGTGCTTGT | G |
| dCas12f | TGTACACCCGCAAG | GCTTGTACACCCGCA | ACACCCGCAAGGTGAGCTGA | |
| native | GTGAGCTGAACATT | AGGTGAGCTGAACAT | ACATTTCACTCACTAAATGCG | |
| | TCACTCACTAAATG | TTCACTCACTAAATG | CTTAGCATATACAACGCGTGG | |
| | CGCTTAGCATATACA | CGCTTAGCATATACAA | GTATCTCTTAATTTCATTAGAA | |
| | ACGCGTGGGTATCT | CGCGTGGGTATCTCT | AAAGATCTTAAGCCGAGCATA | |
| | CTT (SEQ ID NO: | TAATTT (SEQ ID NO: | GTTGAAAACTTAAAAATCTAT | |
| | 6086) | 6096) | CAGCACTTACTATTGCTAGTA | |
| | | | AGGAATTGCCTAATTTAATGC | |
| | | | CTTCCTTCGACGAAAATTTGA | |
| | | | TATTTTTAAATAAGGTTATCCA | |
| | | | ATTTCATTTTTTAAGAACAGT | |
| | | | AAATTTTACAAAGTCGAATGC | |
| | | | AATTAAAATTACCTTTAAGAA | |
| | | | ACTTGATAAGGAGTTCATAAC | |
| | | | CAAATTTCTTACTTATTTTAAA | |
| | | | AAAACAGACTA (SEQ ID NO: | |
| | | | 6106) | |
| Lpa_ | ATCAGAGGGTGCTT | TGACTTTATGATCAG | ACCTATTGATTGACTTTATGAT | (CCN)G |
| dCas12f | GTACACCCGTAAAG | AGGGTGCTTGTACAC | CAGAGGGTGCTTGTACACCC | |
| native | TGAGGTCGCGGGC | CCGTAAAGTGAGGTC | GTAAAGTGAGGTCGCGGGCA | |
| | ACTCACCAAATGCA | GCGGGCACTCACCAA | CTCACCAAATGCAGGAAGCA | |
| | GGAAGCATATACAA | ATGCAGGAAGCATAT | TATACAACGGCGTGTTCATCT | |
| | CGGCGTGTTCATCT | ACAACGGCGTGTTCA | ACACTTAGAAAACAAGAGCT | |
| | ACA (SEQ ID NO: | TCTACACTTA (SEQ ID | TTTTTGTGAGGCATATAAAAT | |
| | 6087) | NO: 6097) | AAACAATGTTGAATTTTCAAT | |
| | | | CTAGAATTTTAAATAGAATCT | |
| | | | GAGGGTACTTGTATACCCGTA | |
| | | | GGGTGAGGCTTTGGCACTCA | |
| | | | CTAAGTAGCATACAGCTTATAT | |
| | | | ACAATGGCGTGAGTTAATTAT | |
| | | | CTTTAAACCGTTTTAAAATGA | |
| | | | TTGTACACAAAAGGTCCTAGT | |
| | | | TTTATCCAGCCTTAATACTATT | |
| | | | AAAAATTATCCCTTGATAAAC | |
| | | | TACACGTTGGGTTAATTGTAA | |
| | | | AAGTTGGTTTAGGTTCCGAAT | |
| | | | GAAGTGTAATCAACAGCTTAT | |
| | | | TTTTAAAGAAAAC (SEQ ID | |
| | | | NO: 6107) | |
| Lby_ | TTTTTTTAGGGGCT | AAAGATTTTTTTAGG | AAAGATTTTTTTAGGGGCTGT | (T)TG |
| dCas12f | GTCCGCACAGCCTT | GGCTGTCCGCACAGC | CCGCACAGCCTTAGGTTGGG | |
| native | AGGTTGGGGCTATT | CATAGGTTGGGGCTA | GCTATTCGCACTCAATAAGTG | |
| | CGCACTCAATAAGT | TTCGCACTCAATAAG | AAAGCATGCGGTTAAGGCGT | |
| | GAAAGCATGCGGTT | TGAAAGCATGCGGTT | GAGAAACCTACATCTAAGCTT | |
| | AAGGCGTGAGAAA | AAGGCGTGAGAAAC | GTAATTCCAGTTTTTGTGGCG | |
| | CC (SEQ ID NO: 6088) | CTACA (SEQ ID NO: | TTTAACTGGACTTTTGGGATT | |
| | | 6098) | ATTTCGGGGATTTATTTTCTTT | |
| | | | TTCAAATGAATTTTCATTTTCC | |
| | | | ATTTGCGCTGATAAAAGTACA | |
| | | | CGTCTACATTTGAAACTATAG | |
| | | | TTAACCTATAGAATAAATATAT | |
| | | | TAAAT (SEQ ID NO: 6108) | |
| Mri_ | CTGTCAATAAAATAT | CTGTCAATAAAATATG | CTGTCAATAAAATATGAGGCT | (G)TG |
| dCas12f | GAGGCTGCTTGTAC | AGGCTGCTTGTACAG | GCTTGTACAGCCGTAGGGTGT | |
| native | AGCCGTAGGGTGTG | CCGTAGGGTGTGGCC | GGCCTTGTTGCCTCACTGAAT | |
| | GCCTTGTTGCCTCA | TTGTTGCCTCACTGA | ATGGATTGAACCTGAGATCAC | |
| | CTGAATATGGATTG | ATATGGATTGAACCT | ATCTGTATATACAACGGCTCG | |
| | AACCTGAGATCACA | GAGATCACATCTGTAT | CTTATCGTGTAAAAATAAGCT | |
| | TCTGTATATACAACG | ATACAACGGCTCGCT | TTCTATAAGTTGTACGGTTAA | |
| | GCTCGCTTATCGTG | TATCGTGTAAAA (SEQ | GTTATAAGCAGAACTTATAGT | |
| | T (SEQ ID NO: 6089) | ID NO: 6099) | (SEQ ID NO: 6109) | |
| Cgl_dCas12f | ATATCGAGGGTGCT | CTGAAAAATATCGAG | CTGAAAAATATCGAGGGTGCT | (A)CCCT |
| native | TGTACACCCGTAGG | GGTGCTTGTACACCC | TGTACACCCGTAGGGTGAGG | |
| | GTGAGGTCTTGGAC | GTAGGGTGAGGTCTT | TCTTGGACACTCACTAAATAC | |
| | ACTCACTAAATACT | GGACACTCACTAAAT | TCTTGTAGCATATACAACGGC | |
| | CTTGTAGCATATACA | ACTCTTGTAGCATATA | GTGGGCTCTTTTACAAAGAA | |
| | ACGGCGTGGGCTCT | CAACGGCGTGGGCTC | AATATGAATTGGCGACTTCAT | |
| | TTT (SEQ ID NO: | TTTTACAAA (SEQ ID | TAAATAGACCCCTTTAATTTT | |
| | 6090) | NO: 6100) | GAGGATCTTGAAAAACACAA | |
| | | | CTTCTCAATTAATTATAAGAA | |
| | | | AGAGGCGGTATTATTTTGTAG | |
| | | | CATAGAATATTTTAGCAGAAA | |
| | | | TCTTTA (SEQ ID NO: 6110) | |
| Zpr_dCas12f | TTAAAAATGAGGGT | AATTAAAAATGAGGG | AATTAAAAATGAGGGTGCTTG | TGA |
| native | GCTTGTACACCCGT | TGCTTGTACACCCGT | TACACCCGTAAGGTGAGGTCT | |
| | AAGGTGAGGTCTCA | AAGGTGAGGTCTCAA | CAATGGCACTCACTAAATGCA | |
| | ATGGCACTCACTAA | TGGCACTCACTAAAT | GGCAGCATATACAACAAAAA | |
| | ATGCAGGCAGCATA | GCAGGCAGCATATAC | CCTTCCCTTTTTTTAGGCATTG | |
| | TACAACAAAAACCT | AACAAAAACCTTCCC | AGGTATGAAGGATGAAGACA | |
| | TCCCT (SEQ ID NO: | TTTTTTTA (SEQ ID | ACTGGAATTATGATGAACAGA | |
| | 6091) | NO: 6101) | AATATTTAGGCTTGGTCATTG | |
| | | | AAAATCAA (SEQ ID NO: 6111) | |
| Cba_dCas12f | ATAACGAGGGTGCT | AAAAAATAACGAGG | AAAAAATAACGAGGGTGCTT | CCN |
| native | TGTACACCCGCAGG | GTGCTTGTACACCCG | GTACACCCGCAGGGTGAGGT | |
| | GTGAGGTGTTGAAC | CAGGGTGAGGTGTTG | GTTGAACACTCACTAAATACT | |
| | ACTCACTAAATACT | AACACTCACTAAATA | CAGTGAGTATATACAACGGCG | |
| | CAGTGAGTATATAC | CTCAGTGAGTATATAC | TGGGCTCTTTTTTTACTTATGT | |
| | AACGGCGTGGGCTC | AACGGCGTGGGCTCT | TTTAAAATACTTCAAACGCTC | |
| | T (SEQ ID NO: 6092) | TTTTTTA (SEQ ID NO: | ATTAATAAATTAAAGGATTACT | |
| | | 6102) | TCAGGGTAATAACTTTTATATT | |
| | | | TAAGGCTCAACCATCTAGTTG | |
| | | | AGCCTTAAATATGAATTACAA | |
| | | | ATGTACTTTTACAGCATCTCT | |
| | | | GTTTTTTAAATACTTATAAATA | |
| | | | GCATTA (SEQ ID NO: 6112) | |
| Pba_dCas12f | TAAAAATACAATTAT | TAAAAATACAATTATG | TAAAAATACAATTATGAGGGT | (CT)TG |
| native | GAGGGTGATTGTAC | AGGGTGATTGTACAC | GATTGTACACCCGTAGGGTGA | |
| | ACCCGTAGGGTGAG | CCGTAGGGTGAGTTT | GTTTGGTTGGCAACACTCACT | |
| | TTTGGTTGGCAACA | GGTTGGCAACACTCA | AAATGCACAGGCATATACAAC | |
| | CTCACTAAATGCAC | CTAAATGCACAGGCA | GCGTGGCTTACTTGTTTTTAA | |
| | AGGCATATACAACG | TATACAACGCGTGGC | TAAGCGTATCAGCAAAAATTG | |
| | CGTGGCTTACTTGT | TTACTTGTTTTTA | CACGAAAAATTTCAGGATTAT | |
| | T (SEQ ID NO: 6093) | (SEQ ID NO: 6103) | TTTTTTGACGATTTATAATCTA | |
| | | | ACGATAAGGAAACTCAATA | |
| | | | (SEQ ID NO: 6113) | |

The scope of the present disclosure is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description. The citation and discussion of such references is provided merely to clarify the description and is not an admission that any reference is prior art to the embodiments described herein. All references cited and discussed in this specification are incorporated herein by reference in their entirety.

1. An engineered system comprising:
a polypeptide comprising a TldR protein, a dCas12f or dCas12f-like protein, and/or a TnpB-transposase fusion protein, or one or more nucleic acids encoding thereof; and
at least one guide RNA, or one or more nucleic acids encoding thereof, wherein the at least one guide RNA is complementary to at least a portion of a target nucleic acid.

2. The engineered system of claim 1, wherein the TldR protein comprises an amino acid sequence having at least 70% identity to any of SEQ ID NOs: 1-508 and 1768-5926, wherein the dCas12f or dCas12f-like protein comprises an amino acid sequence having at least 70% identity to any of SEQ ID NOs: 6026-6042, and/or wherein the TnpB-transposase fusion protein comprises an amino acid sequence having at least 70% identity to any of SEQ ID NOs: 1453-1539.

3. The engineered system of claim 1, wherein the TldR protein and/or the dCas12f or dCas12f-like protein is linked or fused to one or more effector polypeptides.

4. The engineered system of claim 1, wherein the at least one guide RNA is provided on an omega RNA.

5. The engineered system of claim 1, further comprising a donor nucleic acid, wherein the donor nucleic acid is optionally flanked by at least one transposon end sequence.

6. The engineered system of claim 1, further comprising a target nucleic acid.

7. The engineered system of claim 1, wherein the system is a cell-free system.

8. A protein conjugate comprising:
a TldR protein or a dCas12f or dCas12f-like protein; and
one or more effector polypeptides.

9. The protein conjugate of claim 8, wherein the TldR protein comprises an amino acid sequence having at least 70% identity to any of SEQ ID NOs: 1-508 and 1768-5926, or Docket No. COLUM-42528.303 wherein the dCas12f or dCas12f-like protein comprises an amino acid sequence having at least 70% identity to any of SEQ ID NOs: 6026-6042.

10. A composition comprising a system of claim 1.

11. A cell comprising athe system of claim 1.

12. A method for DNA modification comprising contacting a target nucleic acid sequence with a system of claim 1.

13. The method of claim 12, wherein the target nucleic acid sequence is flanked by on the 5′ end by a transposon-adjacent motif (TAM) sequence.

14. The method of claim 12, wherein the target nucleic acid sequence is in a cell and the contacting a target nucleic acid sequence comprises introducing the system into the cell.

15. The method of claim 14, wherein the introducing the system into the cell comprises administering the system to a subject.

16. A composition comprising a protein conjugate of claim 8.

17. A cell comprising a protein conjugate of claim 8.

18. A method for DNA modification comprising contacting a target nucleic acid sequence with a protein conjugate of claim 8.

19. The method of claim 18, wherein the target nucleic acid sequence is in a cell and the contacting a target nucleic acid sequence comprises introducing the system into the cell.

20. The method of claim 19, wherein the introducing the system into the cell comprises administering the system to a subject.
